(19) United States
(12) Patent Application Publication
(10) Pub. No.: US 2023/0250079 A1
(43) Pub. Date: Aug. 10, 2023

(54) SMALL MOLECULE MODULATORS OF IL-17

(71) Applicant: LEO PHARMA A/S, Ballerup (DK)

(72) Inventors: Mark ANDREWS, Ballerup (DK); Morten Dahl SØRENSEN, Ballerup (DK); Mogens LARSEN, Ballerup (DK); Nicolas DEVAUX, Paris (FR); Vinicius BARROS RIBEIRO DA SILVA, Paris (FR); Quentin PERRON, Paris (FR); Xifu LIANG, Ballerup (DK); Jimmi Gerner SEIZBERG, Ballerup (DK)

(21) Appl. No.: 18/001,464

(22) PCT Filed: Jun. 10, 2021
(86) PCT No.: PCT/EP2021/065690

§ 371 Date: Dec. 9, 2022

(30) Foreign Application Priority Data
Jun. 12, 2020 (EP) 20179694.3

(51) Int. Cl. C07D 401/14; C07D 413/14; C07D 405/14; C07D 403/12

(52) U.S. Cl. CPC C07D 401/14; C07D 413/14; C07D 405/14; C07D 403/12

(57) ABSTRACT

The present disclosure relates to a compound according to formula I

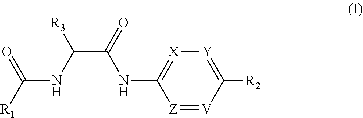

and pharmaceutically acceptable salts, hydrates, or solvates thereof. The disclosure further relates to said compounds for use in therapy, to pharmaceutical compositions comprising said compounds, to methods of treating diseases, e.g. dermal diseases, with said compounds, and to the use of said compounds in the manufacture of medicaments.

### FIELD OF THE INVENTION

This invention relates to novel amino-acid anilides and derivatives thereof, to said compounds for use in therapy and to pharmaceutical compositions comprising said compounds.

### BACKGROUND OF THE INVENTION

IL-17 (also known as IL-17A or CTLA8) is a pro-inflammatory cytokine involved in anti-microbial defense at epithelial surfaces. IL-17 is comprised of two covalently joined IL-17A subunits (IL-17AA) with an approximate mass of 32 kDa, and signals through a receptor comprising IL17RA and IL17RC subunits. This receptor is predominantly expressed in epithelial and mesenchymal cells. The IL17RA/IL17RC receptor is also used by IL-17 variants IL-17AF and IL-17FF, which both are successively weaker, partial agonists on this receptor (Monin, L., Gaffen, S. L.; 2018, Cold Spring Harb. Perspect. Biol. 10. doi:10.1101/cshperspect.a028522). Crucial for signaling is the assembly of signaling complexes containing the multifunctional protein ACT1/CIKS, which in turn can recruit TRAF and other proteins.

Via these signaling complexes IL-17 induces cytokines, chemokines, antimicrobial peptides and growth factors via activation of transcription factor NFkB or via MAP kinase-dependent pathways (e.g. IL-6, IL-8, CXCL1, CXCL2, CXCL5, CCL20, G-CSF, BD4) and stabilizes the mRNAs of certain inflammatory cytokines, such as CXCL1. This leads to amplification of their effects. Further, IL-17 acts in concert with IL-1beta, IL-22 and IFNgamma (Amatya, N. et al., Trends in Immunology, 2017, 38, 310-322. doi:10.1016/j.it.2017.01.006; Onishi, R. M., Gaffen, S. L. Immunology, 2010, 129, 311-321. doi:10.1111/j.1365-2567.2009.03240.x).

IL-17 is secreted by a variety of immune cells, such as Th17 helper cells, Tc17 cytotoxic cells, ILC3 innate cells, NKT cells, TCRbeta+ natural T cells and gamma-deltaT-cells (Monin, L., Gaffen, S. L.; 2018, Cold Spring Harb. Perspect. Biol. 10. doi:10.1101/cshperspect.a028522). Increased, disease-provoking levels of IL-17 are observed in several autoimmune diseases, such as psoriasis, ankylosing spondylitis, spondyloarthritis and psoriatic arthritis. Other diseases where deregulation of IL-17 is observed are rheumatoid arthritis, systemic lupus erythematosus, asthma, inflammatory bowel disease, autoimmune uveitis, multiple sclerosis and certain cancers (Gaffen, S. L. et al., Nat Rev Immunol., 2014, 14, 585-600. doi:10.1038/nri3707; Monin, L., Gaffen, S. L.; 2018, Cold Spring Harb. Perspect. Biol. 10. doi:10.1101/cshperspect.a028522). Hence, IL-17 is a significant therapeutic target.

Therapeutic, neutralizing antibodies against IL-17A (Secukinumab, Ixekizumab) or receptor IL17RA (Brodalumab) have shown high efficacy in the treatment of psoriasis, ankylosing spondylitis and psoriatic arthritis. These antibodies have long half-lives in the body.

Although various antibodies against IL-17A or IL-17RA are approved, there are currently no approved, orally available modulators of IL-17. The following small molecule modulators are known.

WO2013116682 discloses Macrocyclic Compounds for Modulating IL-17;

WO2014066726 discloses Compounds for Modulating IL-17;

WO2018229079 discloses Compounds for Modulating IL-17;

WO2019223718 discloses Compounds for Modulating IL-17;

WO2019138017 discloses Compounds for Modulating IL-17;

WO2020011731 discloses Compounds for Modulating IL-17;

WO2020120140 discloses Compounds for Modulating IL-17;

WO2020120141 discloses Compounds for Modulating IL-17;

WO2020260426 discloses Compounds for Modulating IL-17;

WO2020260425 discloses Compounds for Modulating IL-17;

WO2020261141 discloses Compounds for Modulating IL-17;

WO2020146194 discloses IL-17A inhibitors.

Chinese patent applications CN112341429A, CN112341435A, CN112341439A, CN112341440A, CN112341441A, CN112341442A, CN112341446A, CN112341450A, CN112341451A and CN112341519A disclose Compounds for Modulating IL-17. Scientific Reports (2016) 6, 30859 discloses Macrocyclic IL-17A Antagonists. Leslie Dakin, 12^{th} Swiss Course on Medicinal Chemistry, Leysin, Oct. 9-14, 2016 discloses ‘Hit Identification, binding site elucidation and structure guided design of novel macrocyclic IL-17A antagonists’.

Orally available, highly efficacious small molecule IL-17 modulators which bind to IL-17 to decrease its functional ability to activate the IL-17 receptor complex may have a number of advantages compared to monoclonal antibodies. Oral administration and flexible treatment regimen may be two significant aspects in favor of patient convenience and the compounds may exhibit improved safety due to the possibility of faster withdrawal of the drug should adverse events occur.

Therefore, there is a continuous need to develop small molecule modulators of IL-17, particularly small molecules suitable for oral administration.

In addition, some patients may be treated by topical application of small molecule modulators of IL-17. This can be particularly suitable for patients with skin lesions that are readily accessible and limited in body surface area. Topical treatment may also be prescribed for certain patients who could benefit from avoiding systemic modulation of the IL-17 pathway, for example when undergoing treatment for infections or gastrointestinal problems.

### SUMMARY OF THE INVENTION

The inventors have surprisingly found that novel compounds of the present invention exhibit modulating effects on the IL-17 signalling pathway.

Compounds of the present invention may have advantageous properties such as high metabolic stability and/or membrane permeability properties that make them suitable for oral administration. Other compounds of the present invention may have advantageous properties for local topical therapy, such as high skin permeability and high metabolic instability.

Compounds of the present invention may be beneficial in preventing, treating or ameliorating a variety of diseases which involve up-regulation or de-regulation of IL-17, such as for example psoriasis, ankylosing spondylitis and psoriatic arthritis.

Accordingly, the present invention relates to a compound according to formula (I)

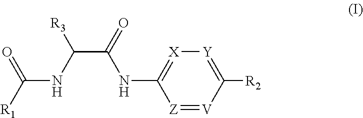

wherein

X, Y, Z and V are each independently selected from N, CH and C(R4); R4 is independently selected from (C₁-C₆)alkyl, (C₁-C₆)alkoxy, hydroxy, NH₂ and halogen, wherein said (C₁-C₆)alkyl and (C₁-C₆)alkoxy may optionally be substituted with one or more substituents independently selected from halogen;

R₁ is selected from the group consisting of (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, 4-6-membered heterocycloalkyl and —NR_{c}R_{d}, wherein said (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, and 4-6-membered heterocycloalkyl is optionally substituted with one or more substituents independently selected from Rₐ;

Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from the group consisting of 5- or 6-membered heteroaryl, wherein said 5- or 6-membered heteroaryl is optionally substituted with one or more substituents independently selected from Rb, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

Rb is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from the group consisting of —CHR₅R₆, (C₃-C₁₀)cycloalkyl and G, wherein said (C₃-C₁₀)cycloalkyl and G are optionally substituted with one or more substituents independently selected from deuterium, halogen, cyano, (C₁-C₄)alkyl and halo(C₁-C₄)alkyl;

G is

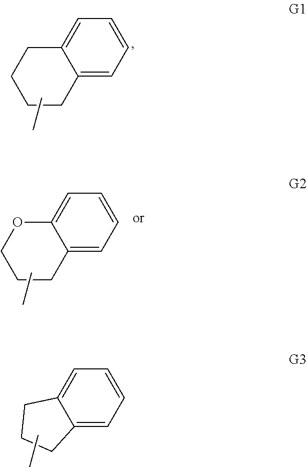

R₅ and R₆ each independently represent hydrogen, phenyl, (C₁-C₆)alkyl, (C₃-C₇)cycloalkyl, and (C₃-C₇)cycloalkyl(C₁-C₆)alkyl wherein said phenyl, (C₁-C₆)alkyl, (C₃-C₇)cycloalkyl and (C₃-C₇)cycloalkyl(C₁-C₆)alkyl is optionally substituted with one or more substituents independently selected from halogen, cyano, and (C₁-C₄)alkyl; with the proviso that at least one of R₅ and R₆ is different from hydrogen;

provided that when R₃ is (C₁-C₄)alkyl, cyclopentyl, cyclohexylmethyl, benzyl or substituted benzyl, then R₁ is selected from the group consisting of pyrazolyl, imidazolyl, thiazolyl, isoxazolyl, oxadiazolyl and triazolyl, wherein the pyrazolyl, imidazolyl, thiazolyl, isoxazolyl, oxadiazolyl or triazolyl is optionally substituted with one or more substituents independently selected from Rₐ; and provided that when all of X, Y, Z and V are C or C(R4) then

Rₐ is (C₁-C₆)alkyl substituted with one or more substituents independently selected from (C₁-C₄)alkyl-S— or (C₁-C₄)alkyl-SO—; or

Rₐ is —NR_{c}R_{d}, wherein R_{c} and R_{d} together form azetidinyl or azetidinyl optionally substituted with one or more substituents independently selected from halogen, cyano and hydroxy; or

Rₐ is 4-6-membered heterocycloalkyl-(C₁-C₆)alkyl wherein said 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d}; or

Rₐ is (C₃-C₇)cycloalkyl-(C₁-C₆)alkyl substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d}; or

R₃ is —CHR₅R₆, wherein at least one of R5 and R6 is (C₃-C₇)cycloalkyl(C₁-C₆)alkyl wherein said (C₃-C₇)cycloalkyl(C₁-C₆)alkyl is optionally substituted with one or more substituents independently selected from halogen, cyano, and (C₁-C₄)alkyl.

or pharmaceutically acceptable salts, hydrates and solvates thereof.

In one embodiment the present invention relates to compounds of formula (Ia)

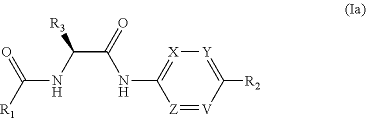

wherein R₁, R₂, R₃, X, Y, Z, V are as defined above; or pharmaceutically acceptable salts, hydrates solvates and prodrugs thereof thereof.

In one embodiment the present invention relates to compounds of formula (Ib)

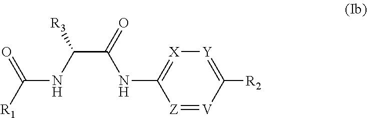

wherein R₁, R₂, R₃, X, Y, Z, and V are as defined in claim 1; or pharmaceutically acceptable salts, hydrates and solvates thereof.

In another aspect, the invention relates to a pharmaceutical composition comprising a compound of general formula (I) as defined herein together with a pharmaceutically acceptable vehicle or excipient or pharmaceutically acceptable carrier(s), optionally together with one or more other therapeutically active compound(s).

In yet another aspect, the invention relates to the use of a compound according to formula I as defined herein for use in therapy, for example for use in treatment of a disease, disorder or condition, which disease, disorder or condition is responsive of modulation of IL-17, for example for use in treatment of autoimmune diseases.

### DETAILED DESCRIPTION OF THE INVENTION

### Definitions

The term “(Cₐ-C_{b})alkyl” is intended to indicate a hydrocarbon radical obtained when one hydrogen atom is removed from a branched or linear hydrocarbon. Said alkyl comprises (a-b) carbon atoms, such as 1-6, such as 1-4, such as 1-3, such as 2-3 or such as 1-2 carbon atoms. The term includes the subclasses normal alkyl (n-alkyl), secondary and tertiary alkyl, such as methyl, ethyl, n-propyl, isopropyl, n-butyl, isobutyl, sec.-butyl, tert.-butyl, n-pentyl, isopentyl, neopentyl, n-hexyl and isohexyl.

The term “(Cₐ-C_{b})alkoxy” is intended to indicate a radical of the formula —OR′, wherein R′ is (Cₐ-C_{b})alkyl as indicated herein, wherein the (Cₐ-C_{b})alkyl group is appended to the parent molecular moiety through an oxygen atom, e.g. methoxy (—OCH₃), ethoxy (—OCH₂CH₃), n-propoxy, isopropoxy, butoxy, tert-butoxy, and the like.

The term “cyano” is intended to indicate a —CN group attached to the parent molecular moiety through the carbon atom.

The term “(Cₐ-C_{b})cycloalkyl” is intended to indicate a saturated (Cₐ-C_{b})cycloalkane hydrocarbon radical, including polycyclic radicals such as bicyclic or tricyclic radicals, including spirocyclic radicals, comprising a-b carbon atoms, such as 3-10 carbon atoms, such as 3-8 carbon atoms, such as 3-7 carbon atoms, such as 3-6 carbon atoms, such as 3-5 carbon atoms or such as 3-4 carbon atoms, e.g. cyclopropyl, cyclobutyl, cyclopentyl, cyclohexyl, cycloheptyl, cyclooctanyl, adamantyl, spiro[2.5]octanyl, spiro[2.3]hexanyl, bicyclo[3,1,0]hexanyl, bicyclo[4,1,0]heptanyl and bicyclo[2,2,2]octanyl.

The term “(Cₐ-C_{b})cycloalkoxy” is intended to indicate a radical of the formula —OR′, wherein R′ is (Cₐ-C_{b})cycloalkyl as indicated herein, wherein the (Cₐ-C_{b})cycloalkyl group is appended to the parent molecular moiety through an oxygen atom, e.g. cyclopentyloxy or cyclobutyloxy.

The term “(Cₐ-C_{b})cycloalkyl(Cₐ-C_{b})alkyl” is intended to indicate an (Cₐ-C_{b})alkyl group as defined herein substituted with one or more (Cₐ-C_{b})cycloalkyl as defined herein, suitably the (Cₐ-C_{b})alkyl group is substituted with one (Cₐ-C_{b})cycloalkyl group.

The term “halo(Cₐ-C_{b})alkyl” is intended to indicate an (Cₐ-C_{b})alkyl group as defined herein substituted with one or more halogen atoms as defined herein, e.g. fluoro or chloro, such as difluoromethyl or trifluoromethyl.

The term “halogen” is intended to indicate a substituent from the 7th main group of the periodic table, such as fluoro, chloro and bromo.

The term “5- or 6-membered heteroaryl” is intended to indicate radicals of monocyclic heteroaromatic rings comprising 5- or 6-membered ring which contains from 1-5 carbon atoms and from 1-4 heteroatoms selected from oxygen, sulphur and nitrogen; such as 2-5 carbon atoms and 1-3 heteroatoms, such as 3-5 carbon atoms and 1-2 heteroatoms, such as 4-5 carbon atoms and 1-2 heteroatoms selected from oxygen, sulphur and nitrogen, such as furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, tetrazolyl, thiadiazolyl, thiazolyl and triazolyl. The term “5- or 6-membered heteroaryl” includes compounds wherein a ring member is a C(O) or carbonyl group.

The term “5-membered heteroaryl” is intended to indicate radicals of 5-membered monocyclic heteroaromatic ring which contains from 1-4 carbon atoms and from 1-4 heteroatoms selected from oxygen, sulphur and nitrogen; such as 2-4 carbon atoms and 1-3 heteroatoms, such as 3-4 carbon atoms and 1-2 heteroatoms, such as 4 carbon atoms and 1 heteroatom selected from oxygen, sulphur and nitrogen; such as furanyl, imidazolyl, isothiazolyl, isoxazolyl, oxadiazolyl, oxazolyl, pyrazolyl, pyrrolyl, tetrazolyl, thiadiazolyl, thiazolyl and triazolyl. The term “5-membered heteroaryl” includes compounds wherein a ring member is a C(O) or carbonyl group.

The term “9- or 10-membered bicyclic heteroaryl” is intended to indicate fused bicyclic heteroaromatic radicals comprising 9- or 10-carbon or heteroatoms, which for example contain from 3-9 carbon atoms and 1-7 heteroatoms selected from oxygen, sulphur and nitrogen, such as 1-5 heteroatoms and 5-9 carbon atoms, such as 1-3 heteroatoms and 7-9 carbon atoms, such as 1-2 heteroatoms and 8-9 carbon atoms, such as 1 heteroatom and 8 carbon atoms, such as 1 heteroatom and 9 carbon atoms, such as 2 heteroatom and 7 carbon atoms, such as 2 heteroatom and 8 carbon atoms. Said bicyclic heteroaromatic radicals comprise a 5- or 6-membered heteroaromatic ring fused to phenyl and a 5- or 6-membered heteroaromatic ring fused to another 5- or 6-membered heteroaromatic ring, as defined herein. The heteroaryl radical may be connected to the parent molecular moiety through a carbon atom or a nitrogen atom contained anywhere within the heteroaryl group. Representative examples of 9- or 10-membered bicyclic heteroaryl include, but are not limited to azaindolyl, benzofuranyl, benzothiophenyl, benzimidazolyl, benzooxazolyl, benzothiazolyl, benzothienyl, cinnolyl, imidazopyridinyl, imidazopyrimidinyl, indazolyl, indolyl, isobenzofuranyl, isoquinolyl, quinolyl, pyrrolopyrimidinyl, thienopyridinyl, pyrrolo[2,3]pyridinyl, pyrrolo[2,3]pyridinyl, pyrazolo[1,5]pyridinyl, pyrazolo[1,5]pyridazinyl, imidazo[1,2]pyrimidinyl, pyrrolo[2,3-c]pyridinyl, pyrrolo[2,3-b]pyridinyl, pyrazolo[1,5-a]pyridinyl, pyrazolo[1,5-b]pyridazinyl, imidazo[1,2-a]pyrimidinyl, imidazo[1,2]pyridazinyl.

The term (5- or 6-membered heteroaryl)-(Cₐ-C_{b})alkyl is intended to indicate a 5- or 6-membered heteroaryl appended to the parent molecular moiety through a (Cₐ-C_{b})alkyl group, as defined herein.

The term “(a-b) membered heterocycloalkyl” is intended to indicate a cycloalkane radical as described herein, including polycyclic radicals such as bicyclic or tricyclic radicals, including spirocyclic radicals, wherein one or more carbon atoms of said cycloalkane radical are replaced by heteroatoms, i.e. the a-b membered heterocycloalkyl comprise from a to b carbon- or hetero-atoms. Such a-b membered heterocycloalkyl could comprise for example 2-9 carbon atoms and 1-6 heteroatoms selected from O, N, or S, such as 3-8 carbon atoms and 1-4 heteroatoms, such as 3-7 carbon atoms and 1-3 heteroatoms, such as 3-6 carbon atoms and 1-2 heteroatom. The heterocycloalkyl radical may be connected to the parent molecular moiety through a carbon atom or a nitrogen atom contained anywhere within the heterocycloalkyl group. Representative examples of heterocycloalkyl groups include, but are not limited to azepanyl, azetidinyl, aziridinyl, dioxolanyl, dioxolyl, imidazolidinyl, morpholinyl, oxetanyl, piperazinyl, piperidinyl, pyrrolidinyl, tetrahydrofuranyl, tetrahydropyranyl, thietanyl, 2,6-diazaspiro[3.3]heptane, 2,6-diazaspiro[3.3]heptanyl, 2,5-diazabicyclo[2.2.1]heptanyl, 2-oxa-5-aza-[2.2.1]heptanyl, 2-oxa-8-azaspiro[3.5]nonanyl, 2-oxa-7-azaspiro[3.5]nonanyl, 2-oxa-8-azaspiro[3.5]nonanyl, 6-oxa-2-azaspiro[3.3]heptanyl, 2-oxa-7-azaspiro[3,4]octanyl, and 1, 3, 3a, 4, 6, 6a-hexahydrofuro[3,4-c]pyrrolyl. The term includes compounds wherein a ring member of said “(a-b) membered heterocycloalkyl” is a C(O) or carbonyl group and S(O) group.

The term “(a-b membered heterocycloalkyl)-(C_{c}-C_{d})alkyl” is intended to indicate a a-b membered heterocycloalkyl radical appended to the parent molecular moiety through an (C_{c}-C_{d})alkyl group, as defined herein.

The term “hydrocarbon radical” is intended to indicate a radical containing only hydrogen and carbon atoms, it may contain one or more double and/or triple carbon-carbon bonds, and it may comprise cyclic moieties in combination with branched or linear moieties. Said hydrocarbon comprises 1-6 carbon atoms, e.g. 1-5, e.g. 1-4, e.g. 1-3, e.g. 1-2 carbon atoms. The term includes alkyl and cycloalkyl as indicated herein.

The term “hydroxy(Cₐ-C_{b})alkyl” is intended to indicate an (Cₐ-C_{b})alkyl group as defined above substituted with one or more hydroxy, e.g. hydroxymethyl, hydroxyethyl, hydroxypropyl.

The term “oxo” is intended to indicate an oxygen atom which is connected to the parent molecular moiety via a double bond (═O).

The term “phenyl-(Cₐ-C_{b})alkyl” is intended to indicate a phenyl group appended to appended to the parent molecular moiety through an (Cₐ-C_{b})alkyl group, as defined herein.

When two or more of the above defined or similar terms are used in combination, such as cycloalkylalkyl or phenyl-(Cₐ-C_{b})alkyl and the like, it is to be understood that the first mentioned radical is a substituent on the latter mentioned radical, where the point of attachment to the parent molecular moiety is on the latter radical.

The group C(O) is intended to represent a carbonyl group (C═O).

If substituents are described as being independently selected from a group, each substituent is selected independent of the other. Each substituent may therefore be identical or different from the other substituent(s).

The term “optionally substituted” means “unsubstituted or substituted”, and therefore the general formulas described herein encompasses compounds containing the specified optional substituent(s) as well as compounds that do not contain the optional substituent(s).

As used herein whenever a molecular drawing of a substituent contains an arrow—the arrow indicates the bond attaching the substituent to the rest of the molecule.

The term “pharmaceutically acceptable salt” is intended to indicate salts prepared by reacting a compound of formula I, which comprise a basic moiety, with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxyethanesulfonic acid, toluenesulfonic, sulfamic or fumaric acid.

Pharmaceutically acceptable salts of compounds of formula I comprising an acidic moiety may also be prepared by reaction with a suitable base such as sodium hydroxide, potassium hydroxide, magnesium hydroxide, calcium hydroxide, zinc hydroxide, barium hydroxide, ammonia or the like, or suitable non-toxic amines, such as lower alkylamines (such as diethylamine, tetraalkylammonium hydroxide), hydroxy-lower alkylamines (such as diethanolamine, 2-(diethylamino)-ethanol, ethanolamine, triethanolamine, tromethamine, deanol), cycloalkylamines, ethylene diamine, or benzylamines, (such as benethamine and benzathine), betaine, choline hydroxide, N-methyl-glucamine, hydrabamine, 1H-imidazole, 4-(2-hydroxyethyl)-morpholine, piperazine, 1-(2-hydroxyethyl)-pyrrolidine, L-arginine or L-lysine. Further examples of pharmaceutical acceptable salts are listed in Berge, S. M.; J. Pharm. Sci.; (1977), 66(1), 1-19, and Stahl, P. H. and in Wermuth, C. G, Handbook of Pharmaceutical Salts, Properties, Selection and Use, 2^{nd} Edition, Wiley-VCH, 2011 both of which are incorporated herein by reference. For example when R₈ is -L-PO(OH)₂ the phosphoric acid group may form a salt with a monovalent cation M⁺ or divalent cation Q²⁺ to form a group selected from -L-PO(OH)O⁻.M⁺, -L-PO(OH)O⁻.½Q²⁺-L-PO(O⁻)₂.2M⁺, and -L-PO(O⁻)₂.Q²⁺.

The term ‘monovalent cation’ is intended to indicate monovalent cations such as alkali metal ions, such as for example sodium (Nat), potassium (K⁺) or lithium (Li⁺), or ammonium ions, such as for example NH₄⁺, dialkylammonium (NH₂((C₁-C₄)alkyl)₂)⁺, trialkylammonium (NH((C₁-C₄)alkyl)₃)+, or tetraalkylammonium (N((C₁-C₄)alkyl)₄)⁺, alkylammonium (H₃N(C₁-C₄)alkyl)⁺ or hydroxyalkylammonium (H₃N-hydroxy(C₁-C₄)alkyl)⁺, the protonated forms of L-arginine, L-lysine or the protonated forms of any pharmaceutically acceptable bases such as those mentioned above.

The term ‘divalent cation’ is intended to indicate divalent cations such as alkaline earth metal ions such as calcium (Ca²⁺), Magnesium (Mg²⁺), barium (Ba₂₊), or Zinc (Zn₂₊).

The term ‘prodrug’ is intended to indicate compounds which are drug-precursors which, upon administration, are converted to the parent drug in vivo by enzymatic and/or chemical reactions. Generally, the pro-drug is less biologically active than its parent drug. The prodrug may have improved physical-chemical properties compared to the parent drug, such as improved aqueous solubility, thereby facilitating the absorption and consequently the bioavailability of the parent compound upon administration.

The term ‘parent drug’ or ‘parent compound’ is intended to indicate the biologically active compound which is released from the prodrug via enzymatic and/or chemical processes following administration of the prodrug. The parent drug is frequently the starting material for the preparation of the corresponding prodrug.

Examples of prodrugs according to the invention are prodrugs that are attached to a nitrogen or oxygen of the parent molecule.

For example when the parent molecule contains a 5-membered heteroaryl containing nitrogen substituted with hydrogen as a ring atom said hydrogen may be replaced with a substituent selected from -L-PO(OH)₂, wherein L is selected from the group consisting of a bond or —CHR_{g}O— and R_{g} is selected from hydrogen and (C₁-C₆)alkyl to form a prodrug.

5-membered heteroaryls such as pyrrole, imidazole, pyrazole, triazole and tetrazole when attached to the reminder of the molecule via a carbon ring atom are moieties that may contain a nitrogen ring atom substituted by hydrogen.

The term “solvate” is intended to indicate a species formed by interaction between a compound, e.g. a compound of formula I, and a solvent, e.g. alcohol, glycerol or water, wherein said species are in a crystalline form. When water is the solvent, said species is referred to as a hydrate.

The term “or pharmaceutically acceptable salts, hydrates and solvates thereof” includes compound of formula (I) and hydrates or solvates thereof, and pharmaceutically acceptable salts of the compounds of formula (I) as well as hydrates or solvates thereof.

The term “treatment” as used herein means the management and care of a patient for the purpose of combating a disease, disorder or condition. The term is intended to include the delaying of the progression of the disease, disorder or condition, the amelioration, alleviation or relief of symptoms and complications, and/or the cure or elimination of the disease, disorder or condition. The term may also include prevention of the condition, wherein prevention is to be understood as the management and care of a patient for the purpose of combating the disease, condition or disorder and includes the administration of the active compounds to prevent the onset of the symptoms or complications. Nonetheless, prophylactic (preventive) and therapeutic (curative) treatments are two separate aspects.

All references, including publications, patent applications and patents, cited herein are hereby incorporated by reference in their entirety and to the same extent as if each reference were individually and specifically indicated to be incorporated by reference, regardless of any separately provided incorporation of particular documents made elsewhere herein.

According to one embodiment, the invention relates to a compound of formula (I), (Ia) or (Ib) wherein X, Y, Z and V are each independently selected from N, CH and C(R₄); provided that at least one of X, Y, Z and V is N.

According to another embodiment, the invention relates to a compound of formula (I), (Ia) or (Ib) wherein

R₁ is selected from pyrazolyl, imidazolyl, thiazolyl, isoxazolyl, oxadiazolyl and triazolyl, wherein the pyrazolyl, imidazolyl, thiazolyl, isoxazolyl, oxadiazolyl and triazolyl is optionally substituted with one or more substituents independently selected from Rₐ.

Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from the group consisting of 5- or 6-membered heteroaryl, wherein said 5- or 6-membered heteroaryl is optionally substituted with one or more substituents independently selected from Rb, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from —CHR₅R₆, and wherein R₅ and R₆ each independently represent hydrogen, phenyl, cyclopropyl, cyclobutyl, cyclopentyl, cyclohexyl, cyclopropylmethyl, cyclobutylmethyl, methyl or ethyl, wherein said phenyl, cyclopropyl, cyclobutyl, cyclopentyl, cyclohexyl, cyclopropylmethyl, cyclobutylmethyl, methyl or ethyl, is optionally substituted with one or more substituents independently selected from halogen, cyano, and (C₁-C₄)alkyl; with the proviso that at least one of R5 and R6 is different from hydrogen;

X, Y, Z and V are each independently selected from N, CH and C(R₄); provided that at least one of X, Y, Z and V is N;

R₄ is independently selected from (C₁-C₆)alkyl, (C₁-C₆)alkoxy, hydroxy, NH₂ and halogen; wherein said (C₁-C₆)alkyl and (C₁-C₆)alkoxy may optionally be substituted with one or more substituents independently selected from halogen;

or pharmaceutically acceptable salts, hydrates and solvates thereof.

According to another embodiment, the invention relates to a compound of formula (I), (Ia) or (Ib), wherein

R₁ is selected from pyrazolyl, imidazolyl, thiazolyl, isoxazolyl, oxodiazolyl and triazolyl, wherein said pyrazolyl, imidazolyl, thiazolyl, isoxazolyl, oxodiazolyl and triazolyl is optionally substituted with one or more substituents independently selected from Rₐ.

Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from the group consisting of 5- or 6-membered heteroaryl, wherein said 5- or 6-membered heteroaryl is optionally substituted with one or more substituents independently selected from R_{b}, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, -NR_{c}d and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from —CHR₅R₆, and wherein R₅ and R₆ each independently represent (C₃-C₇)cycloalkyl, or (C₃-C₇)cycloalkyl(C₁-C₆)alkyl, wherein said (C₃-C₇)cycloalkyl or (C₃-C₇)cycloalkyl(C₁-C₆)alkyl is optionally substituted with one or more substituents independently selected from halogen, cyano and (C₁-C₄)alkyl;

X, Y, Z and V are each independently selected from N, CH and C(R4); provided that at least one of X, Y, Z and V is N;

R₄ is independently selected from (C₁-C₆)alkyl, (C₁-C₆)alkoxy, hydroxy, NH₂ and halogen; wherein said (C₁-C₆)alkyl and (C₁-C₆)alkoxy may optionally be substituted with one or more substituents independently selected from halogen;

or pharmaceutically acceptable salts, hydrates and solvates thereof.

According to another embodiment, the invention relates to a compound of formula (I), (Ia) or (Ib), wherein

R₁ is selected from pyrazolyl, imidazolyl, thiazolyl, isoxazolyl, oxodiazolyl and triazolyl, wherein said pyrazolyl, imidazolyl, thiazolyl, isoxazolyl, oxadiazolyl and triazolyl is optionally substituted with one or more substituents independently selected from Rₐ.

Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from the group consisting of 5- or 6-membered heteroaryl, wherein said 5- or 6-membered heteroaryl is optionally substituted with one or more substituents independently selected from R_{b}, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, -NR_{c}R_{d} and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from cyclohexyl, cycloheptyl, cyclooctanyl, adamantyl, spiro[2.3]hexanyl, bicyclo[3,1,0]hexanyl, bicyclo[4,1,0]heptanyl, bicyclo[2,2,2]octanyl or spiro[2.5]octanyl, wherein said cyclohexyl, cycloheptyl, cyclooctanyl, adamantyl, spiro[2.3]hexanyl, bicyclo[3,1,0]hexanyl, bicyclo[4,1,0]heptanyl, bicyclo[2,2,2]octanyl or spiro[2.5]octanyl is optionally substituted with one or more substituents independently selected from deuterium, halogen, cyano, (C₁-C₄)alkyl and halo(C₁-C₄)alkyl;

X, Y, Z and V are each independently selected from N, CH and C(R4); provided that at least one of X, Y, Z and V is N;

R₄ is independently selected from (C₁-C₆)alkyl, (C₁-C₆)alkoxy, hydroxy, NH₂ and halogen; wherein said (C₁-C₆)alkyl and (C₁-C₆)alkoxy may optionally be substituted with one or more substituents independently selected from halogen;

or pharmaceutically acceptable salts, hydrates and solvates thereof.

According to another embodiment, the invention relates to a compound of formula (I), (Ia) or (Ib) wherein

R₁ is selected from pyrazolyl, imidazolyl, thiazolyl, isoxazolyl, oxodiazolyl and triazolyl, wherein said pyrazolyl, imidazolyl, thiazolyl, isoxazolyl, oxadiazolyl and triazolyl is optionally substituted with one or more substituents independently selected from Rₐ.

Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from the group consisting of 5- or 6-membered heteroaryl, wherein said 5- or 6-membered heteroaryl is optionally substituted with one or more substituents independently selected from R_{b}, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, -NR_{c}d and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from G, wherein G is

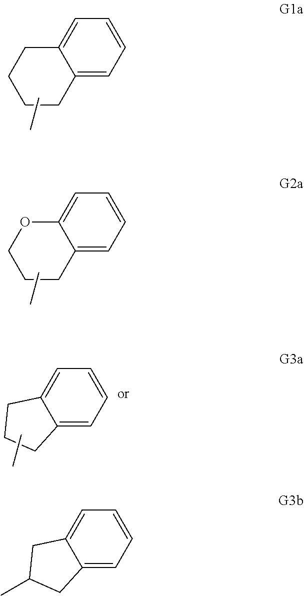

wherein said G is optionally substituted with one or more substituents independently selected from deuterium, halogen, cyano, (C₁-C₄)alkyl and halo(C₁-C₄)alkyl.

X, Y, Z and V are each independently selected from N, CH and C(R4); provided that at least one of X, Y, Z and V is N;

R₄ is independently selected from (C₁-C₆)alkyl, (C₁-C₆)alkoxy, hydroxy, NH₂ and halogen; wherein said (C₁-C₆)alkyl and (C₁-C₆)alkoxy may optionally be substituted with one or more substituents independently selected from halogen;

or pharmaceutically acceptable salts, hydrates and solvates thereof.

According to one embodiment, the invention relates to a compound of formula (I), (Ia) or (Ib), wherein R₂ is selected from pyrazolyl and imidazolyl, wherein said pyrazolyl or imidazolyl is optionally substituted with one or more substituents independently selected from R_{b}.

According to one embodiment, the invention relates to a compound of formula (I), (Ia) or (Ib), wherein R₂ is pyrazol-4-yl or imidazole-4-yl, wherein said pyrazol-4-yl or imidazol-4-yl is substituted with one or more substituents independently selected from (C₁-C₆)alkyl or deuterated (C₁-C₄)alkyl.

According to one embodiment, the invention relates to a compound of formula (I), (Ia) or (Ib), wherein R₂ is selected from pyrazol-4-yl or imidazole-4-yl, wherein said pyrazol-4-yl or imidazol-4-yl contain a nitrogen ring atom substituted by a substituent selected from -L-PO(OH)₂ and the other ring atoms of said pyrazol-4-yl or imidazole-4-yl is substituted with one or more substituents independently selected from (C₁-C₆)alkyl or deuterated (C₁-C₄)alkyl.

According to one embodiment, the invention relates to a compound of formula (I), (Ia) or (Ib), wherein R₁ is pyrazolyl or triazolyl wherein said pyrazolyl or triazolyl is optionally substituted with one or more substituents independently selected from (C₁-C₆)alkyl and (C₃-C₇)cycloalkyl-(C₁-C₆)alkyl wherein said one or more (C₁-C₆)alkyl and (C₃-C₄)cycloalkyl-(C₁-C₂)alkyl is optionally substituted with one or more substituents independently selected from halogen, hydroxy, (C₁-C₄)alkoxy, (C₁-C₄)alkyl-S—, (C₁-C₄)alkyl-SO—, and (C₁-C₄)alkyl-SO₂—.

According to one embodiment, the invention relates to a compound of formula (I), (Ia) or (Ib), wherein R₁ is pyrazol-3-yl or 1,2,3-triazol-4-yl substituted with one substituent selected from (C₁-C₄)alkyl and (C₃-C₄)cycloalkyl-(C₁-C₂)alkyl wherein said (C₁-C₄)alkyl and (C₃-C₄)cycloalkyl-(C₁-C₂)alkyl is optionally substituted with a substituent selected from halogen, hydroxy, (C₁-C₄)alkoxy, (C₁-C₄)alkyl-S—, (C₁-C₄)alkyl-SO— and (C₁-C₄)alkyl-SO₂—, R₂ is pyrazol-4-yl substituted with one or more (C₁-C₄)alkyl or deuterated (C₁-C₄)alkyl, R₃ is —CHR₅R₆, wherein R₅ and R₆ each independently represent (C₃-C₇)cycloalkyl.

According to one embodiment, the invention relates to a compound of formula (I), (Ia) or (Ib), wherein R₁ is 2-(C₁-C₃)alkyl-pyrazol-3-yl, R₂ is 3,5-di(C₁-C₂)alkyl-pyrazol-4-yl, R₃ is —CHR₅R₆, wherein R₅ and R₆ each independently represent (C₃-C₄)cycloalkyl.

According to one embodiment, the invention relates to a compound of formula (I), (Ia) or (Ib), wherein

X is N and Y, Z and V are independently selected from CH and C(R₄),

Y is N and X, Z and V are independently selected from CH and C(R₄),

X and Y are N and V and Z are independently selected from CH and C(R₄),

Y and Z are N and X and V are independently selected from CH and C(R₄),

X and Z are N and Y and V are independently selected from CH and C(R₄), or

Y and V are N and X and Z are independently selected from CH and C(R₄).

According to one specific embodiment, the invention relates to a compound of formula (I), (Ia) or (Ib), wherein X is N, Y is C(R₄) and V and Z are CH.

According to one embodiment, the invention relates to a compound of formula (I), (Ia) or (Ib), wherein

R₁ is selected from pyrazolyl, imidazolyl, thiazolyl, isoxazolyl, oxadiazolyl and triazolyl, wherein said pyrazolyl, imidazolyl, thiazolyl, isoxazolyl, oxadiazolyl and triazolyl is optionally substituted with a substituent independently selected from Rₐ.

Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from pyrazolyl and imidazolyl, wherein said pyrazolyl or imidazolyl is optionally substituted with one or more substituents independently selected from R_{b};

R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, -NR_{c}d and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from —CHR₅R₆, and wherein R₅ and R₆ each independently represent (C₃-C₇)cycloalkyl, wherein said (C₃-C₇)cycloalkyl is optionally substituted with one or more substituents independently selected from halogen, cyano, and (C₁-C₄)alkyl;

X is N, Y is C(R₄) and V and Z are CH;

R₄ is selected from (C₁-C₆)alkyl, (C₁-C₆)alkoxy, hydroxy, NH₂ and halogen; wherein said (C₁-C₆)alkyl and (C₁-C₆)alkoxy may optionally be substituted with one or more substituents independently selected from halogen;

or pharmaceutically acceptable salts, hydrates and solvates thereof.

According to one embodiment, the invention relates to the embodiment above, wherein R₁ is pyrazol-3-yl or 1,2,3-triazol-4-yl substituted with one or more (C₁-C₄)alkyl or (C₃-C₄)cycloalkyl-(C₁-C₂)alkyl wherein said (C₁-C₄)alkyl or (C₃-C₄)cycloalkyl-(C₁-C₂)alkyl may optionally be substituted with one or more substituents selected from halogen, (C₁-C₄)alkoxy, (C₁-C₄)alkyl-S—, (C₁-C₄)alkyl-SO—, (C₁-C₄)alkyl-SO₂—, R₂ is pyrazol-4-yl substituted with one or more (C₁-C₄)alkyl or deuterated (C₁-C₄)alkyl, R₃ is —CHR₅R₆, and wherein R₅ and R₆ each independently represent (C₃-C₇)cycloalkyl, and X is N, Y is C(R₄), wherein R₄ is halogen and V and Z are CH.

According to one embodiment, the invention relates to the embodiment above, wherein R₁ is 2-(C₁-C₃)alkyl-pyrazol-3-yl, R₂ is 3,5-di(C₁-C₂)alkyl-pyrazol-4-yl, R₃ is —CHR₅R₆, and wherein R₅ and R₆ each independently represent (C₃-C₄)cycloalkyl, and X is N, Y is C(R₄), wherein R₄ is fluoro and V and Z are CH.

According to one embodiment, the invention relates to a compound of formula (I), (Ia) or (Ib) wherein X, Y, Z and V are selected from C and C(R₄), and Rₐ is (C₁-C₆)alkyl substituted with one or more substituents independently selected from (C₁-C₄)alkyl-S— or (C₁-C₄)alkyl-SO—.

According to one embodiment, the invention relates to a compound of formula (I), (Ia) or (Ib) wherein X, Y, Z and V are selected from C and C(R₄), and Rₐ is —NR_{c}R_{d}, wherein R_{c} and R_{d} together form azetidinyl or azetidinyl optionally substituted with one or more substituents independently selected from halogen, cyano and hydroxy.

According to one embodiment, the invention relates to a compound of formula (I), (Ia) or (Ib) wherein X, Y, Z and V are selected from C and C(R₄), and Rₐ is 4-6-membered heterocycloalkyl-(C₁-C₆)alkyl wherein said 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d}.

According to one embodiment, the invention relates to a compound of formula (I), (Ia) or (Ib) wherein X, Y, Z and V are selected from C and C(R₄), and Rₐ is (C₃-C₇)cycloalkyl-(C₁-C₆)alkyl substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d}.

According to one embodiment, the invention relates to a compound of formula (I, (Ia) or (Ib) wherein X, Y, Z and V are selected from C and C(R₄), and R₃ is —CHR₅R₆, wherein at least one of R₅ and R₆ is (C₃-C₇)cycloalkyl(C₁-C₆)alkyl wherein said (C₃-C₇)cycloalkyl(C₁-C₆)alkyl is optionally substituted with one or more substituents independently selected from halogen, cyano, and (C₁-C₄)alkyl.

According to one embodiment, the invention relates to a compound of formula (I) or (Ia) wherein R₃ is —CHR₅R₆, wherein at least one of R₅ and R₆ is (C₃-C₇)cycloalkyl(C₁-C₆)alkyl wherein said C₃-C₇)cycloalkyl(C₁-C₆)alkyl is optionally substituted with one or more substituents independently selected from halogen, cyano, and (C₁-C₄)alkyl.

In one or more embodiments of the present invention, the compounds of general formula I have an (EC₅₀) value in an IL-8 release assay of less than 1 micromolar, or of less than 100 nanomolar.

The compounds of formula I may be obtained in crystalline form either directly by concentration from an organic solvent or by crystallisation or recrystallisation from an organic solvent or mixture of said solvent and a co-solvent that may be organic or inorganic, such as water. The crystals may be isolated in essentially solvent-free form or as a solvate, such as a hydrate. The invention covers all crystalline forms, such as polymorphs and pseudopolymorphs, and also mixtures thereof.

Compounds of formula I comprise asymmetrically substituted (chiral) carbon atoms which give rise to the existence of isomeric forms, e.g. enantiomers and possibly diastereomers. The present invention relates to all such isomers, either in optically pure form or as mixtures thereof (e.g. racemic mixtures or partially purified optical mixtures). Pure stereoisomeric forms of the compounds and the intermediates of this invention may be obtained by the application of procedures known in the art. The various isomeric forms may be separated by physical separation methods such as selective crystallization and chromatographic techniques, e.g. high pressure liquid chromatography using chiral stationary phases. Enantiomers may be separated from each other by selective crystallization of their diastereomeric salts which may be formed with optically active amines, or with optically active acids. Optically purified compounds may subsequently be liberated from said purified diastereomeric salts. Enantiomers may also be resolved by the formation of diastereomeric derivatives. Alternatively, enantiomers may be separated by chromatographic techniques using chiral stationary phases. Pure stereoisomeric forms may also be derived from the corresponding pure stereoisomeric forms of the appropriate starting materials, provided that the reaction occur stereoselectively or stereospecifically. Preferably, if a specific stereoisomer is desired, said compound will be synthesized by stereoselective or stereospecific methods of preparation. These methods will advantageously employ chiral pure starting materials.

Furthermore, when a double bond or a fully or partially saturated ring system is present in the molecule geometric isomers may be formed. Any geometric isomer, as separated, pure or partially purified geometric isomers or mixtures thereof are included within the scope of the invention.

In the compounds of general Formula I, the atoms may exhibit their natural isotopic abundances, or one or more of the atoms may be artificially enriched in a particular isotope having the same atomic number, but an atomic mass or mass number different from the atomic mass or mass number found in nature. The present invention includes all suitable isotopic variations of the compounds of general Formula I. For example, different isotopic forms of hydrogen include 1H, 2H and 3H, different isotopic forms of carbon include ¹²C, ¹³C and ¹⁴C and different isotopic forms of nitrogen include ¹⁴N and ¹⁵N. Enriching for deuterium (²H) may for example increase in-vivo half-life or reduce dosage regimens, or may provide a compound useful as a standard for characterization of biological samples. Isotopically enriched compounds within general formula I can be prepared by conventional techniques well known to a person skilled in the art or by processes analogous to those described in the general procedures and examples herein using appropriate isotopically enriched reagents and/or intermediates.

Some compounds have lower aqueous solubility which may affect the absorption and consequently the bioavailability of the compounds. Such compounds may advantageously be administered in the form of prodrugs improving the aqueous solubility of the parent compound. Such prodrugs which, upon administration, are converted to their parent compounds may be less active in vitro compared to their parent compounds, but because of the improved aqueous solubility, facilitating the absorption and consequently the bioavailability of the parent compounds upon administration, such prodrugs have improved in vivo activity compared to their parent compounds.

Prodrugs of the compounds of formula (I) form part of the invention claimed.

Solvates and hydrates form part of the invention claimed.

The compounds of the present invention may be useful for preventing, treating or ameliorating any of the following diseases: psoriasis, ankylosing spondylitis, spondyloarthritis or psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, rheumatoid arthritis, Pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer.

In an embodiment the invention relates to the use of a compound of general formula (I) as defined above, in the manufacture of a medicament for the prophylaxis, treatment or amelioration of any of the following diseases: psoriasis, ankylosing spondylitis, spondyloarthritis or psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, rheumatoid arthritis, Pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer.

In an embodiment the invention relates to the use of a compound of general formula (I) as defined above, in the manufacture of a medicament for the prophylaxis, treatment or amelioration of autoimmune diseases, such as psoriasis, ankylosing spondylitis, spondyloarthritis or psoriatic arthritis.

In an embodiment the invention relates to a method of preventing, treating or ameliorating autoimmune diseases, such as psoriatic arthritis, lichen planus, lupus nephritis, Sjögren's syndrome, acne, vitiligo, alopecia areata, ichthyosis, acute and chronic liver diseases, gout, osteoarthritis, SLE (besides LN and DLE), multiple sclerosis, plaque psoriasis, pustular psoriasis, rheumatoid arthritis, Pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer, the method comprising administering to a person suffering from at least one of said diseases an effective amount of one or more compounds according to general formula (I), optionally together with a pharmaceutically acceptable carrier or one or more excipients, optionally in combination with other therapeutically active compounds.

In an embodiment the invention relates to a method of preventing, treating or ameliorating autoimmune diseases, such as psoriasis, ankylosing spondylitis, spondyloarthritis or psoriatic arthritis, the method comprising administering to a person suffering from at least one of said diseases an effective amount of one or more compounds according to general formula (I), optionally together with a pharmaceutically acceptable carrier or one or more excipients, optionally in combination with other therapeutically active compounds.

Besides being useful for human treatment, the compounds of the present invention may also be useful for veterinary treatment of animals including mammals such as horses, cattle, sheep, pigs, dogs, and cats.

### Pharmaceutical Compositions of the Invention

For use in therapy, compounds of the present invention are typically in the form of a pharmaceutical composition. The invention therefore relates to a pharmaceutical composition comprising a compound of formula I, optionally together with one or more other therapeutically active compound(s), together with a pharmaceutically acceptable excipient, vehicle or carrier(s). The excipient must be “acceptable” in the sense of being compatible with the other ingredients of the composition and not deleterious to the recipient thereof.

Conveniently, the active ingredient comprises from 0.0001-99.9% by weight of the formulation.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 0.001 mg and 1000 mg, preferably between 0.01 mg and 300 mg of a compound of formula I.

A suitable dosage of the compound of the invention will depend, inter alia, on the age and condition of the patient, the severity of the disease to be treated and other factors well known to the practising physician. The compound may be administered either orally, parenterally, topically, transdermally or intradermally and other routes according to different dosing schedules, e.g. daily, weekly or with monthly intervals. In general a single dose will be in the range from 0.001 to 400 mg/kg body weight.

If the treatment involves administration of another therapeutically active compound it is recommended to consult Goodman & Gilman's The Pharmacological Basis of Therapeutics, 9^{th}, Ed., J. G. Hardman and L. E. Limbird (Eds.), McGraw-Hill 1995, for useful dosages of said compounds.

The administration of a compound of the present invention with one or more other active compounds may be either concomitantly or sequentially.

The formulations include e.g. those in a form suitable for oral, rectal, parenteral transdermal, intradermal, ophthalmic, topical, nasal, sublingual or buccal administration.

The formulations may conveniently be presented in dosage unit form and may be prepared by but not restricted to any of the methods well known in the art of pharmacy, e.g. as disclosed in Remington, The Science and Practice of Pharmacy, 21ed ed., 2005. All methods include the step of bringing the active ingredient into association with the carrier, which constitutes one or more accessory ingredients. In general, the formulations are prepared by uniformly and intimately bringing the active ingredient into association with a liquid carrier, semisolid carrier or a finely divided solid carrier or combinations of these, and then, if necessary, shaping the product into the desired formulation.

Formulations of the present invention suitable for oral and buccal administration may be in the form of discrete units as capsules, sachets, tablets, chewing gum or lozenges, each containing a predetermined amount of the active ingredient.

A tablet may be made by compressing, moulding or freeze drying the active ingredient optionally with one or more accessory ingredients. Compressed tablets may be prepared by compressing, in a suitable machine, the active ingredient(s) in a free-flowing form; for example with a lubricant; a disintegrating agent or a dispersing agent. Moulded tablets may be made by moulding, in a suitable machine, a mixture of the powdered active ingredient and suitable carrier. Freeze dried tablets may be formed in a freeze-dryer from a solution of the drug substance.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. Liposomal formulations are also suitable for parenteral administration.

Transdermal formulations may be in the form of a plaster, patch, microneedles, liposomal or nanoparticulate delivery systems or other cutaneous formulations applied to the skin.

Formulations suitable for ophthalmic administration may be in the form of a sterile aqueous preparation of the active ingredients. Liposomal formulations or biodegradable polymer systems may also be used to present the active ingredient for ophthalmic administration.

Formulations suitable for topical, such as dermal, intradermal or ophthalmic administration include liquid or semi-solid preparations, solutions or suspensions.

Formulations suitable for nasal or buccal administration include powder, self-propelling and spray formulations, such as aerosols and atomisers.

Methods of Preparation

The compounds of the present invention can be prepared in a number of ways well known to those skilled in the art of synthesis. The compounds of the invention could for example be prepared using the reactions and techniques outlined below together with methods known in the art of synthetic organic chemistry, or variations thereof as appreciated by those skilled in the art. Preferred methods include, but are not limited to, those described below. The reactions are carried out in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. Not all compounds falling into a given class may be compatible with some of the reaction conditions required in some of the methods described. Such restrictions to the substituents which are compatible with the reaction conditions will be readily apparent to one skilled in the art and alternative methods can be used.

The compounds of the present invention or any intermediate could be purified, if required, using standard methods well known to a synthetic organist chemist, e.g. methods described in “Purification of Laboratory Chemicals”, 6^{th} ed. 2009, W. Amarego and C. Chai, Butterworth-Heinemann.

Starting materials are either known or commercially available compounds, or may be prepared by routine synthetic methods well known to a person skilled in the art.

Unless otherwise noted, reagents and solvents were used as received from commercial suppliers. The organic solvents used were usually anhydrous. The solvent ratios indicated refer to vol:vol unless otherwise noted. Thin layer chromatography was performed using Merck 6OF254 silica-gel TLC plates. Visualisation of TLC plates was performed using UV light (254 nm) or by an appropriate staining technique.

Proton nuclear magnetic resonance spectra were obtained at the stated frequencies in the solvents indicated. Tetramethylsilane was used as an internal standard for proton spectra. The value of a multiplet, either defined doublet (d), triplet (t), quartet (q) or (m) at the approximate midpoint is given unless a range is quoted. (br) indicates a broad peak, whilst (s) indicates a singlet.

Mass spectra were obtained using the following methods. LCMS Method 1 was used, unless otherwise stated.

LCMS Method 1:

Column: Acquity UPLC HSS T3 1.8 μm; 2.1×50 mm

Flow: 0.7 mL/min

Column temp: 30° C.

Mobile phases: A: 10 mM Ammonium acetate+0.1% formic acid, B: 100% Acetonitrile+0.1% formic acid

UV: 240-400 nm

Injection volume: 1 μl

Gradient:

| Time (min) | A % | B % |
|---|---|---|
| 0.0 | 99% | 1% |
| 0.5 | 94% | 6% |
| 1.0 | 94% | 6% |
| 2.6 | 5% | 95% |
| 3.8 | 5% | 95% |
| 3.81 | 99% | 1% |
| 4.8 | 99% | 1% |

UPLC (inlet method): XEV Metode 1 CM

MS—method: Pos_50_1000 or Neg_50_1000

Instruments: Waters Acquity UPLC, Waters XEVO G2-XS QTof, Waters PDA (Photodiode Array)

LCMS Method 2:

Mass spectra were obtained on a Waters Quattro micro API/Waters SQD2/Waters Quattro Premier Spectrometer using electrospray ionization and atmospheric-pressure chemical ionization with the column and solvents indicated.

LCMS Method 3:

Column: Waters Acquity UPLC HSS T3 1.8 μm, 2.1×50 mm.

Column temperature: 60° C.

UV: PDA 210-400 nm.

Injection volume: 2 μl.

Eluents: A: 10 mM Ammonium acetate with 0.1% formic acid, B: 100% Acetonitrile with 0.1% formic acid.

Gradient:

| Time (min) | A % | B % | Flow (mL/min) |
|---|---|---|---|
| 0.0 | 95 | 5 | 1.2 |
| 0.9 | 5 | 95 | 1.2 |
| 0.91 | 5 | 95 | 1.3 |
| 1.2 | 5 | 95 | 1.3 |
| 1.21 | 5 | 95 | 1.2 |
| 1.4 | 95 | 5 | 1.2 |

MS: Electrospray switching between positive and negative ionisation.

Instruments: Waters ACQUITY, Waters SQD, Waters PDA (Photodiode array)

LCMS Method 4:

Column: Waters ACQUITY BEH 1.7 μm, 2.1×50 mm.

Column temperature: 60° C.

UV: PDA 210-400 nm.

Injection volume: 2 μl.

Eluents: A: 10 mM Ammonium Bicarbonate, B: 100% Acetonitrile

Gradient:

| Time (min) | % A | % B | Flow (mL/min) |
|---|---|---|---|
| 0.0 | 95 | 5 | 1.2 |
| 0.9 | 5 | 95 | 1.2 |
| 0.91 | 5 | 95 | 1.3 |
| 1.2 | 5 | 95 | 1.3 |
| 1.21 | 5 | 95 | 1.2 |
| 1.4 | 95 | 5 | 1.2 |

MS: Electrospray positive or negative ionisation.

Instruments: Waters ACQUITY, Waters QDa (MS detector), Waters PDA (Photodiode Array)

Basic preparative HPLC conditions:

Column: XBridge Prep C18 5 μm OBD, 19×150 mm

Eluents: Ammonium formate (50 mM)/acetonitrile, 10-100% acetonitrile

Flow: 30 mL/min

Acidic preparative HPLC conditions:

Column: XTerra® RP-18 5 μm OBD, 19×150 mm

Eluents: 0.1% formic acid in water/acetonitrile, 10-100% acetonitrile

Flow: 30 mL/min

The following abbreviations have been used throughout:
ABPR automated back pressure regulator
AcOH acetic acid
Boc tert-butoxycarbonyl
BOP (benzotriazol-1-yloxy)tris(dimethylamino)phosphonium hexafluorophosphate
CAN ceric ammonium nitrate
CBz benzyloxycarbonyl
CDI carbonyldiimidazole
CPME cyclopentyl methyl ether
DABCO 1,4-diazabicyclo[2.2.2]octane
DAST (diethylamino)sulfur trifluoride
DBU 1,8-diazabicyclo[5.4.0]undec-7-ene
DEA diethylamine
DEAD diethyl azodicarboxylate
DCC dicyclohexylcarbodiimide
DCM dichloromethane
DIAD diisopropyl azodicarboxylate
DIBAL diisobutylaluminium hydride
DIPEA diisopropylethylamine
DMF N,N-dimethylformamide
DMSO dimethylsulfoxide
dppf 1,1′-bis(diphenylphosphino)ferrocene
EDC N-(3-dimethylaminopropyl)-N′-ethylcarbodiimide
FA formic acid
EtOAc ethyl acetate
EtOH ethanol
HATU 1-[bis(dimethylamino)methylene]-1H-1,2,3-triazolo[4,5-b]pyridinium 3-oxide hexafluorophosphate
HBTU N,N,N′,N′-tetramethyl-O-(1H-benzotriazol-1-yl)uronium hexafluorophosphate
HPLC high-performance liquid chromatography
IPA isopropyl alcohol
LCMS liquid chromatography—mass spectrometry
LiHMDS lithium bis(trimethylsilyl)amide
MCPBA meta-chloroperbenzoic acid
Me methyl
MeCN acetontitrile
MeOH methanol
MHz megahertz
NBS N-bromosuccinimide
NMP N-methyl-2-pyrrolidinone
NMR nuclear magnetic resonance
ppm parts per million
Prep. preparation
Prep. HPLC preparative HPLC
PyBOP (benzotriazol-1-yloxy)tripyrrolidinophosphonium hexafluorophosphate
RT retention time
SEM 2-(trimethylsilyl)ethoxymethyl
SFC supercritical fluid chromatography
SM starting material
Soln solution
TBME tert-butyl methyl ether
TFA trifluoroacetic acid
THF tetrahydrofuran
TLC thin layer chromatography
TMEDA tetra methylethylenediamine
T3P propanephosphonic acid anhydride

General Methods

Compounds of the invention may be prepared according to the following non-limiting general methods and examples:

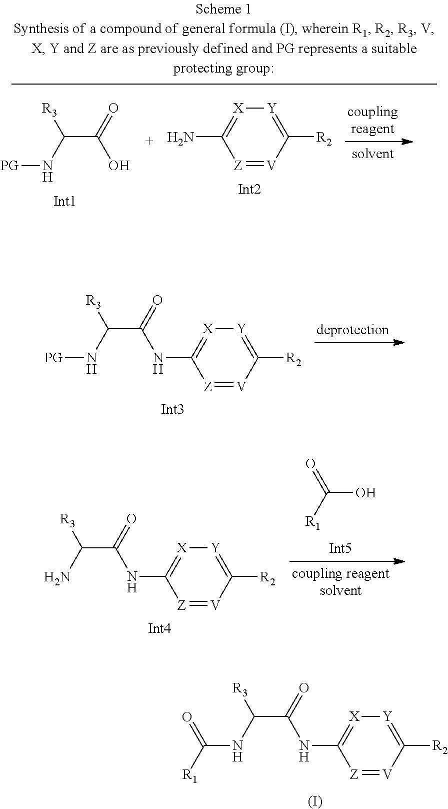

Compounds of general formula (I) can be prepared, as shown in Scheme 1. Compounds of general formula (Int 1), which are either commercially available or are synthesised in a racemic form or an enantiomerically pure form, are coupled with amines of general formula (Int 2), which are either commercially available or synthesised, in the presence of a coupling reagent such as T3P, CDI, DCC, HATU, HBTU and EDC and in the majority of cases, in the presence of a base, such as DIPEA or TEA, in a suitable solvent, such as DMF or acetonitrile to form compounds of formula (Int 3). Protecting groups (PG), such as Boc, or Cbz, on compounds of general formula (Int 3) can be removed or selectively removed by methods known to those skilled in the art. Compounds of general formula (Int 4) are coupled with compounds of general formula (Int 5), which are either commercially available or synthesised, in the presence of a coupling reagent such as HATU, HBTU, CDI, T3P, PyBOP, BOP, DCC or EDC and in most of the cases in the presence of a base, such as DIPEA or triethylamine, in a suitable solvents, such as DMF or acetonitrile to form compounds of general formula (I). Where the compounds of general formula (I) contain protecting groups, those protecting groups can be removed by methods known to those skilled in the art. Racemic compounds of general formula (Int 3), (Int 4) or (I) can be separated by chiral SFC, to give the S-enantiomers of compounds of general formula (Int 3), (Int 4) or (I).

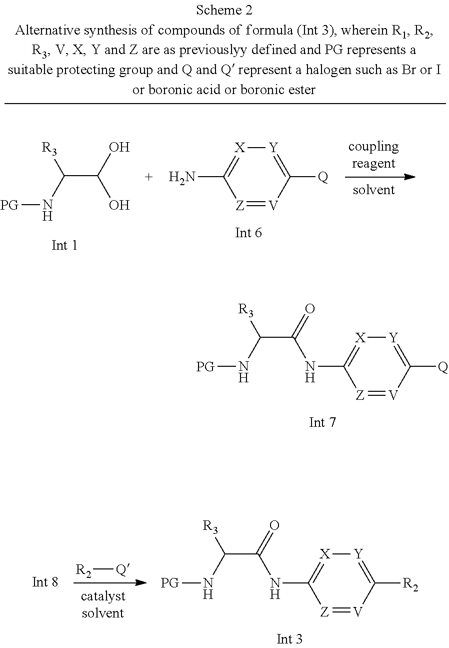

Compounds of general formula (Int 3) can be prepared as shown in Scheme 2. Compounds of general formula (Int 1), which are either commercially available or are synthesised in a racemic form or an enantiomerically pure form, are coupled with amines of general formula (Int 6), which are either commercially available or synthesised, in the presence of a coupling reagent such as T3P, CDI, DCC, HATU, HBTU and EDC and in the majority of cases, in the presence of a base, such as DIPEA or TEA, in a suitable solvent, such as DMF or acetonitrile to form compounds of formula (Int 7). Compounds of general formula (Int 8), where Q′ is Br, I, boronic acid or boronic ester, that are either commercially available or are synthesised, can be reacted with compounds of formula (Int 7). Compounds of formula (Int 8) may contain protecting groups that can be removed or selectively removed by methods known to those skilled in the art. The reaction takes place in the presence of a catalyst such as [1,1′-bis(diphenylphosphino)ferrocene]palladium(II) dichloride, PdCl₂(dppf), or bis(triphenylphosphine)palladium(II) dichloride, PdCl₂(PPh₃)₂, in the presence of an aqueous base, such as K₂CO₃ or Na₂CO₃, in a suitable solvent, such as DMF or toluene to form compounds of formula (Int3). Those skilled in the art will appreciate other metal mediated coupling reaction will give rise to compounds of general formula (Int 3).

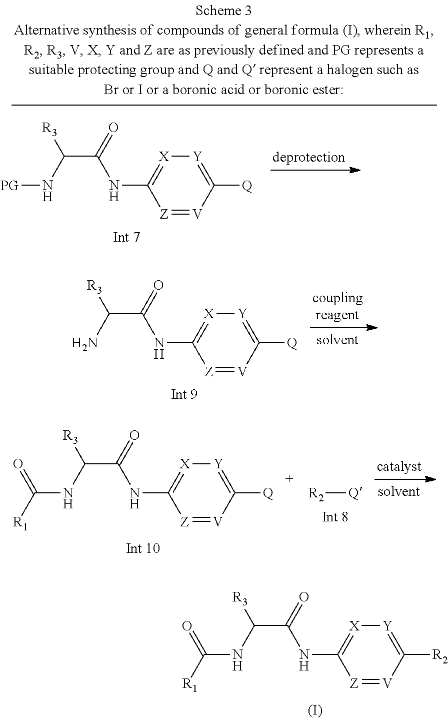

Compounds of general formula (I) can be prepared as shown in Scheme 3. Protecting groups (PG), such as Boc, or Cbz, on compounds of general formula (Int 7) can be removed or selectively removed by methods known to those skilled in the art. Compounds of general formula (Int 9), which are synthesised in a racemic form or an enantiomerically pure form, are coupled with compounds of general formula (Int 5), which are either commercially available or synthesised, in the presence of a coupling reagent such as HATU, HBTU, CDI, T3P, PyBOP, BOP, DCC or EDC and in most of the cases in the presence of a base, such as DIPEA or triethylamine, in a suitable solvent, such as DMF or acetonitrile to form compounds of general formula (Int 10). Compounds of general formula (Int 10) may be reacted with compounds of formula (Int 8). Compounds of general formula (Int 8) may contain protecting groups that can be removed or selectively removed to those skilled in the art. The reaction takes place in the presence of a catalyst such as [1,1′-bis(diphenylphosphino)ferrocene]palladium(II) dichloride PdCl₂(dppf), or bis(triphenylphosphine)palladium(II) dichloride, PdCl₂(PPh₃)₂, in the presence of an aqueous base, such as K₂CO₃ or Na₂CO₃, in a suitable solvent, such as DMF or toluene to form compounds of formula (I). Those skilled in the art will appreciate other metal mediated coupling reaction will give rise to compounds of general formula (I).Where the compounds of general formula (I) contain protecting groups, those protecting groups can be removed by methods known to those skilled in the art. Racemic compounds of general formula (Int 9), (Int 10) or (I) can be separated by chiral SFC, to give the S-enantiomers of compounds of general formula (Int 9), (Int 10) or (I).

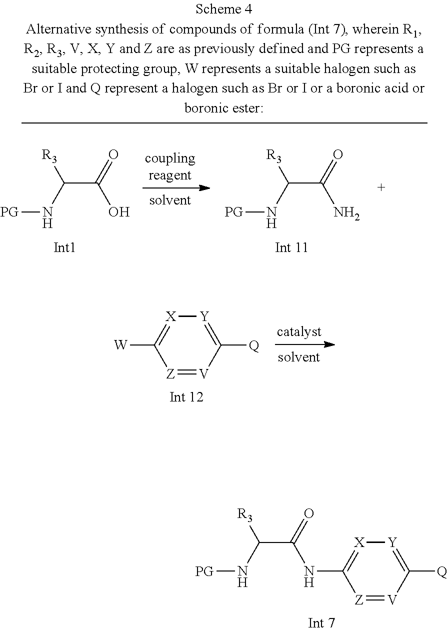

Compounds of general formula (Int 7) can be prepared as shown in Scheme 4. Compounds of general formula (Int 1), which are either commercially available or are synthesised in a racemic form or an enantiomerically pure form, are reacted with an ammonia equivalent, such as ammonium chloride, in the presence of a coupling reagent such as T3P, CDI, DCC, HATU, HBTU and EDC and in the majority of cases, in the presence of a base, such as DIPEA or TEA, in a suitable solvent, such as DMF or acetonitrile or reacted with ammonium bicarbonate in the presence of tert-butoxycarbonyl tert-butyl carbonate and pyridine in a solvent such as 1,4-dioxane to form compounds of formula (Int 11). Compounds of formula (Int 11) can be reacted with compounds of formula (Int 12) in the presence of palladium (II) acetate or tetrakis(triphenylphosphine)palladium(0) and 4,5-bis(diphenylphosphino)-9,9-dimethylxanthene and a base such as K₂CO₃ OR Cs₂CO₃ in a solvent such as THF or DMF, to form compounds of formula (Int 7).

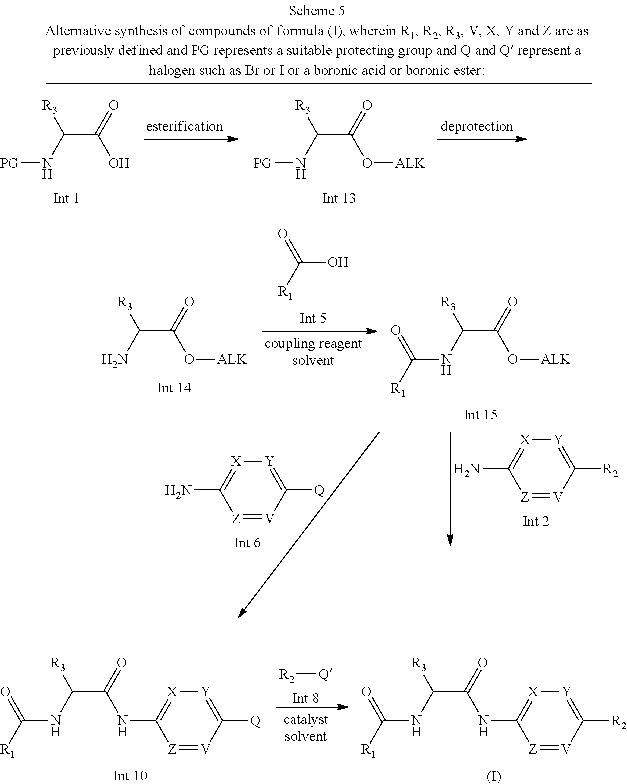

Compounds of general formula (I) can be prepared as shown in Scheme 5. Compounds of general formula (Int 1), which are either commercially available or are synthesised in a racemic form or an enantiomerically pure form, are coupled with an alcohol, generally methanol or ethanol in the presence of EDC and DMAP in a suitable solvent such as DCM, to give compounds of general formula (Int 13). Protecting groups (PG), such as Boc, or Cbz, on compounds of general formula (Int 13) can be removed or selectively removed by methods known to those skilled in the art. Compounds of general formula (Int 14) are coupled with compounds of general formula (Int 5), which are either commercially available or synthesised, in the presence of a coupling reagent such as HATU, HBTU, CDI, T3P, PyBOP, BOP, DCC or EDC and in most of the cases in the presence of a base, such as DIPEA or triethylamine, in a suitable solvent, such as DMF or acetonitrile to form compounds of general formula (Int 15). Compounds of general formula (Int 15) can be reacted with compounds of general formula (Int 2) or (Int 6) in the presence of trimethylaluminium in a suitable solvent such as toluene to give compound of general formula (Int 10) or (I). Where the compounds of general formula (I) contain protecting groups, those protecting groups can be removed by methods known to those skilled in the art.

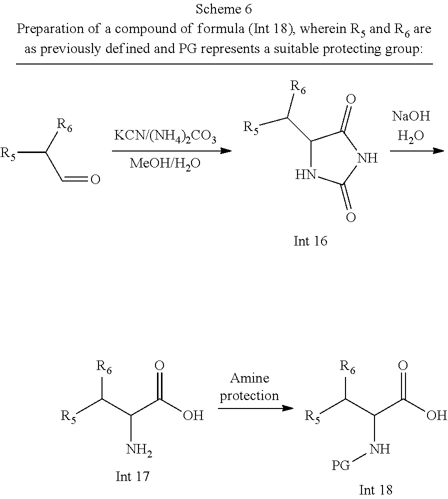

Compounds of formula (Int 18) can be prepared as shown in Scheme 6. The reaction of an aldehyde with potassium cyanide and ammonium carbonate in water and methanol forms compounds of formula (Int 16) (For Bucherer Bergs reaction, see: Chemical Reviews 2017 117 (23), 13757-13809). Compounds of formula (Int 17) can be prepared by treatment of compounds of formula (Int 16) with alkali hydroxides such as sodium hydroxide or potassium hydroxide in water. The amines of formula (Int18) can be formed by methods known to those skilled in the art using, for example, CbzCl or Boc anhydride.

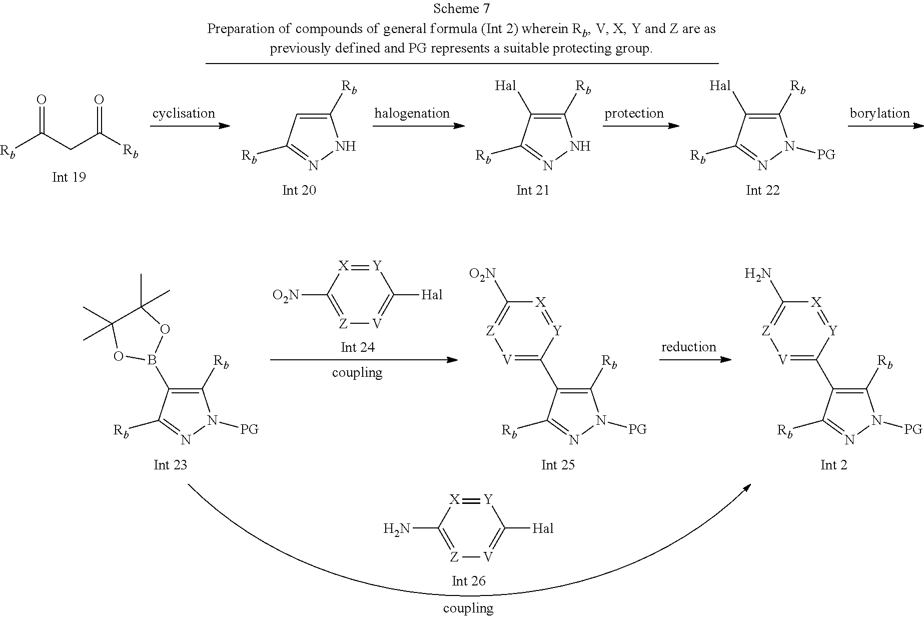

Compounds of general formula (Int 2) can be prepared as shown in Scheme 7. Compounds of general formula (Int 19), which are either commercially available or are synthesized, can be reacted with hydrazine hydrate in the presence of AcOH in a suitable solvent such as EtOH or MeOH to give compounds of general formula (Int 20). Reaction of these with reagents such as NIS or NBS in a suitable solvent such as MeCN, gives compounds of general formula (Int 21). The compounds of formula (Int 22) can be synthesised by methods known to those skilled in the art using, for example, using SEMCI or Boc anhydride. Compounds of general formula (Int 22) can be reacted to give compounds of general formula (Int 23) either in the presence of bis(pinacolato)diboron, a catalytic palladium source such as [1,11-bis(diphenylphosphino)ferrocene]palladium(II) dichloride PdCl₂(dppf), a base such as K₂CO₃ in a suitable solvent such as DMF or MeCN or in the presence of 2-isopropoxy-4,4,5,5-tetramethyl-1,3,2-dioxaborolane and a suitable base such as n-butyllithium in a suitable solvent such as THF. Compounds of general formula (Int 24) or (Int 26) can be reacted with compounds of general formula (Int 23) in the presence of palladium source such as [1,1′-bis(diphenylphosphino)ferrocene]palladium(II) dichloride PdCl₂(dppf), or bis(triphenylphosphine)palladium(II) dichloride, PdCl₂(PPh₃)₂, in the presence of an aqueous base, such as K₂CO₃ or Na₂CO₃, in a suitable solvent, such as DMF or toluene to give compounds of general formula (Int 2) or (Int 25). Reduction of the nitro group in compounds of general formula (Int 25) can be carried out by many methods known to those skilled in the art to give anilines of general formula (Intl). For example, by catalytic hydrogenation, using a suitable catalyst, such as Pd on carbon, in a suitable solvent, such as EtOAc, MeOH or IPA, under a suitable pressure of hydrogen.

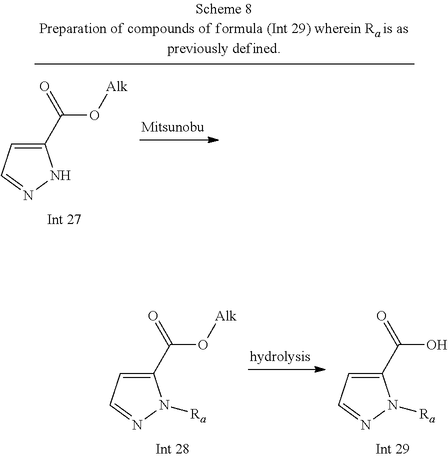

Compounds of general formula (Int 29) can be prepared as shown in Scheme 8. Compounds of formula (Int 27) that are commercial or synthesized can be reacted with alcohols, that are commercial or synthesized, under Mitsunobu conditions, namely in the presence of a phosphine such as triphenylphosphine and a diazodicarboxylate such as DEAD or DIAD, in a suitable solvent such as toluene or THF, to give compounds of formula (Int 28). Those skilled in the art will appreciate that some of the embodiments of Rₐ will undergo literature precedented transformation or deprotection, before hydrolysis with an appropriate base such as LiOH or NaOH in a suitable solvent such as MeOH or THF, to give compounds of general formula (Int 29).

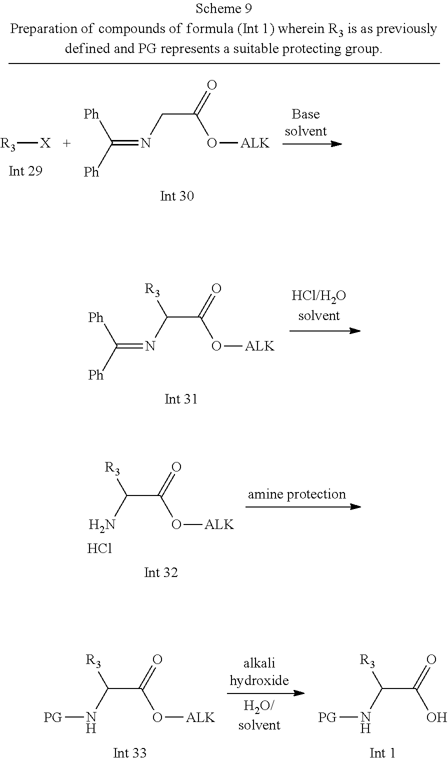

Compounds of general formula (Int 1) can be prepared, as shown in Scheme 9. Compounds of formula (Int 29) are reacted with commercially available imines (Int 30) in the presence of a suitable base, typically an alkali metal carbonate, such as sodium carbonate, potassium carbonate or cesium carbonate in a suitable solvent such as DMSO, DMF or acetonitrile to form compounds of formula (Int 31). Hydrolysis of compounds of formula (Int 31) can be performed by using aqueous HCl in a suitable solvent, such as THF, to give compounds of general formula (Int 32). The amines of formula (Int 32) can be protected by methods known to those skilled in the art. The esters of formula (Int 33) are readily converted to compounds of general formula (Int 1) in the presence of an alkali hydroxide such as sodium hydroxide, potassium hydroxide or lithium hydroxide. Racemic compounds of general formula (Int 33) can be separated by chiral SFC, to give the S-enantiomers of compounds of general formula (Int 33).

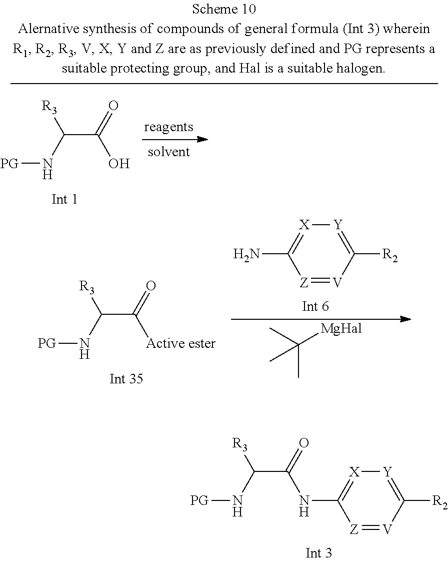

Compounds of general formula (Int 3) can be prepared as shown in Scheme 10. Compounds of general formula (Int 1), which are either commercially available or are synthesised in a racemic form or an enantiomerically pure form, are reacted to form activated esters of general formula (Int 35). Typically this could be a reaction of a compound of general formula (Int 1) with (2,3,4,5,6-pentafluorophenyl) 2,2,2-trifluoroacetate in a solvent such as DCM, in the presence of a suitable base such as pyridine or triethylamine in a solvent such as MeCN or DCM, or with 1-hydroxypyrrolidine-2,5-dione in the presence of a coupling reagent such as EDC or DCC in a suitable solvent such as DCM or THF. Compounds of general formula (Int 35) can be reacted with compounds of general formula (Int 6) in the presence of suitable alkylmagnesium halides such as ^{t}BuMgCl or ^{t}BuMgBr, in a suitable solvent such as THF, to give the compounds of general formula (Int 3).

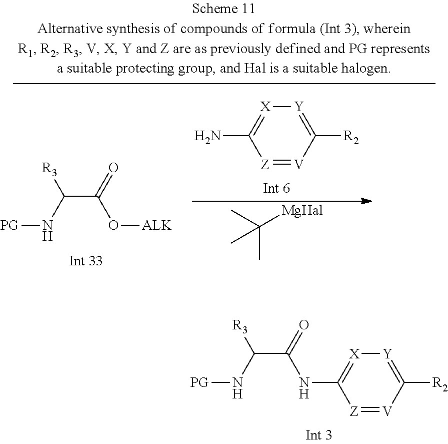

Compounds of general formula (Int 3) can be prepared as shown in Scheme 11. Compounds of general formula (Int 33) can be reacted with with compounds of general formula (Int 6) in the presence of suitable alkylmagnesium halides such as ^{t}BuMgCl or ^{t}BuMgBr, in a suitable solvent such as THF, to give the compounds of general formula (Int 3).

### PREPARATIONS AND EXAMPLES

### Preparations

### Preparation 1: (1-cyclopropyl-2-methoxy-vinyl)cyclopropane

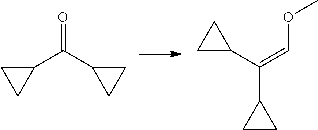

n-Butyllithium (2.5 M solution in heptanes, 26 mL, 65.6 mmol) was added slowly to a suspension of methoxymethyl(triphenyl)phosphonium chloride (22.5 g, 65.6 mmol) in dry THF (130 mL) at 5° C. under argon. The resulting deep red solution was stirred for 20 min, then dicyclopropylmethanone (5 mL, 4.82 g, 43.8 mmol) was added and the reaction mixture was stirred overnight at 60° C. under argon. The reaction mixture was allowed to cool to room temperature, concentrated in vacuo and the residue was purified by dry-flash chromatography (silica gel, eluting with hexane). Crude title compound (5.69 g, 94%) was isolated as a clear oil which was used without further purification. 1H NMR (300 MHz, CDCl₃) δ 5.86 (dd, J=1.6, 0.7 Hz, 1H), 3.57 (s, 3H), 1.87-1.74 (m, 1H), 0.89-0.78 (m, 1H), 0.76-0.67 (m, 2H), 0.64-0.57 (m, 2H), 0.51-0.41 (m, 2H), 0.27-0.19 (m, 2H).

### Preparation 2: 2,2-dicyclopropylacetaldehyde

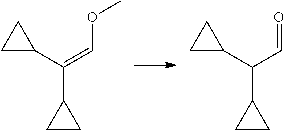

The compound of Preparation 1 (5.6 g, 41 mmol) was dissolved in THF (20 mL) and 6M HCl (20 mL) was added. The mixture was stirred vigorously for 1 week at room temperature. The reaction mixture was extracted with ether (2×50 mL), dried (Na₂SO₄) and carefully evaporated. Crude 2,2-dicyclopropylacetaldehyde (2.80 g, 56%) was isolated as a pale yellow oil which was used directly in the following step without any further purification.

### Preparation 3: 2-(tert-butoxycarbonylamino)-3,3-dicyclopropyl-propanoic acid

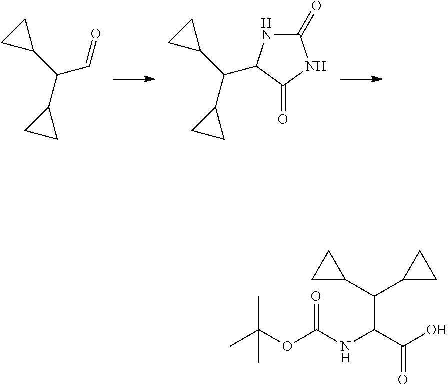 [SMILES: O=CC(C1CC1)C1CC1 ; O=C1NC(=O)C(C(C2CC2)C2CC2)N1 ; CC(C)(C)OC(=O)NC(C(=O)O)C(C1CC1)C1CC1]

The compound of Preparation 2 (2.80 g, 22.5 mmol) was placed in a 20 mL microwave vial with KCN (2.20 g, 33.8 mmol) and ammonium carbonate (6.50 g, 67.6 mmol) in MeOH:water (8 mL:8 mL). The vial was capped and stirred at 60° C. (conventional heating) for 2 days to give a brown mixture with some precipitation. 4M HCl was added until the pH was less than 5. After cooling to room temperature the brown solid was filtered off, washed with water (3 mL) and dried to give crude hydantoin (4.38 g, 22.6 mmol) that was used without further purification.

The crude hydantoin (4.38 g, 22.6 mmol) was heated at reflux in 5M NaOH (30 mL) overnight, then cooled in an ice bath and 5M HCl (20 mL) was added slowly. THF (30 mL) was added followed by tert-butoxycarbonyl tert-butyl carbonate (4.93 g, 22.6 mmol. The mixture was stirred at room temperature for 1 hour then 5M HCl was added carefully until the pH was between 3 and 4. The mixture was extracted with EtOAc (3×50 mL) and the combined organic extracts were dried (Na₂SO₄) and evaporated. Purification by column chromatography (silica gel, eluting with EtOAc:heptane) gave the title compound (1.32 g, 22%) as a pale yellow oil. 1H NMR (300 MHz, CDCl₃) Mixture of rotamers δ 7.90 (br s, 1H), 5.78 (br, 0.15H), 5.26 (d, J=9.2 Hz, 0.85H), 4.55 (d, J=9.2 Hz, 0.85H), 4.37 (br, 0.15H), 1.46 (s, 9H), 1.33-1.21 (m, 1H), 0.85-0.64 (m, 2H), 0.61-0.36 (m, 4H), 0.32-0.13 (m, 4H); LCMS (METHOD 3) (ES): m/z 268.4 [M−H]⁻, RT=0.70 min.

### Preparation 4: tert-butyl N-[1-[(5-bromo-2-pyridyl)carbamoyl]-2,2-dicyclopropyl-ethyl]carbamate

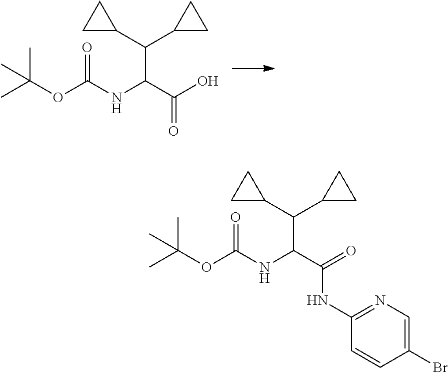 [SMILES: CC(C)(C)OC(=O)NC(C(=O)O)C(C1CC1)C1CC1 ; CC(C)(C)OC(=O)NC(C(=O)Nc1ccc(Br)cn1)C(C1CC1)C1CC1]

EDAC (1.067 g, 5.57 mmol) was added to a solution of the product from Preparation 3 (1.00 g, 3.71 mmol), 5-bromopyridin-2-amine (706 mg, 4.08 mmol) and DMAP (499 mg, 4.08 mmol) in DCM (10 mL). The reaction mixture was stirred at 40° C. for 2 hours. The reaction mixture was partitioned between DCM (20 mL) and water (10 mL). The organic phase was washed successively with NaHSO₄ (10% aqueous solution, 10 mL) and brine (10 mL), then dried over MgSO₄, filtered and concentrated in vacuo. The obtained crude compound was purified by silica column chromatography (230-400 mesh), eluting with EtOAc (0-100%) in heptane, to afford the title compound as a colourless solid (573 mg, 36% yield). 1H NMR (400 MHz, DMSO-d6) δ 10.58 (s, 1H), 8.43 (dd, J=2.4, 0.9 Hz, 1H), 8.17-7.92 (m, 2H), 7.11-6.50 (m, 1H), 4.47-4.21 (m, 1H), 1.39 (s, 9H), 0.98-0.67 (m, 2H), 0.62-−0.01 (m, 9H). LCMS (METHOD 3) (ES): m/z 426.1 [M−H]⁻, RT=0.91 min.

### Preparation 5: 2-[(4-bromo-5-ethyl-3-methyl-pyrazol-1-yl)methoxy]ethyl-trimethyl-silane

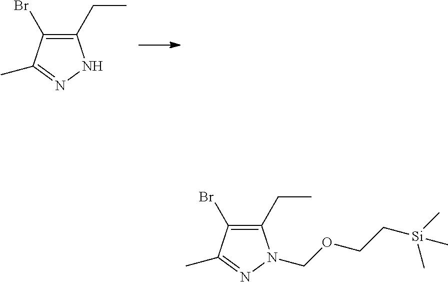

SEM chloride (2.95 mL, 16.7 mmol) was added to a solution of 4-bromo-5-ethyl-3-methyl-1H-pyrazole (2.1 g, 11.1 mmol) and Cs₂CO₃ (9.05 g, 27.8 mmol) in DMF (22 mL) and stirred for 16 hours at room temperature. The reaction mixture was diluted with Et₂O (100 mL) and washed with H₂O (2×30 mL). The organic layer was dried over MgSO₄, filtered and dried in vacuo. The obtained crude compound was purified by silica column chromatography (230-400 mesh), eluting with EtOAc (0-100%) in heptane, to afford the title compound as a mixture of regioisomers. (2.1 g, 59% yield). 1H NMR (400 MHz, DMSO-d6) δ 5.48-5.20 (m, 2H), 3.67-3.41 (m, 2H), 2.80-2.41 (m, 2H), 2.31-1.98 (m, 3H), 1.24-0.66 (m, 5H), -0.01-−0.16 (m, 9H). (approx. 6:1 ratio of regioisomers).

### Preparation 6: 2-[[5-ethyl-3-methyl-4-(4,4,5,5-tetramethyl-1,3,2-dioxaborolan-2-yl)pyrazol-1-yl]methoxy]ethyl-trimethyl-silane

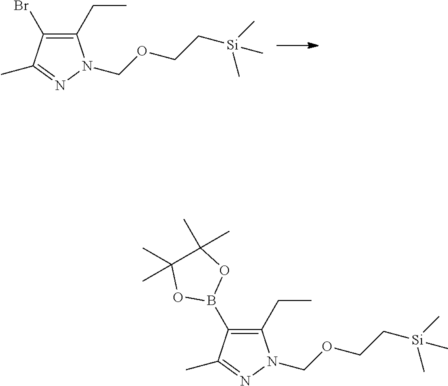

n-Butyllithium (32.0 mL, 81.1 mmol, 2.5M solution) was added dropwise to a solution of the product from Preparation 5 (18.5 g, 57.9 mmol) in anhydrous THF (250 mL) at −75° C. The reaction mixture was stirred at −75° C. for 15 min. 2-Isopropoxy-4,4,5,5-tetramethyl-1,3,2-dioxaborolane (14.0 mL, 68.6 mmol) was added and the solution was warmed to room temperature over 45 min. The reaction mixture was quenched with saturated NH₄Cl solution (50 mL) and extracted with EtOAc (2×150 mL). The combined organic extracts were dried over Na₂SO₄, filtered and concentrated in vacuo. The obtained crude compound was purified by silica column chromatography (230-400 mesh), eluting with EtOAc in heptane, to afford the title compound as a colourless oil. (18.8 g, 88% yield); LCMS (METHOD 3) (ES): m/z 367.3 [M+H]⁺, RT=1.08 min. (approx. 6:1 ratio of regioisomers).

### Preparation 7: tert-butyl N-[1-(dicyclopropylmethyl)-2-[[5-[5-ethyl-3-methyl-1-(2-trimethylsilylethoxymethyl)pyrazol-4-yl]-2-pyridyl]amino]-2-oxo-ethyl]carbamate

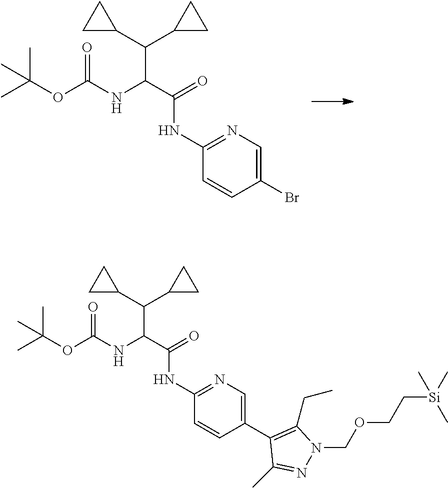 [SMILES: CC(C)(C)OC(=O)NC(C(=O)Nc1ccc(Br)cn1)C(C1CC1)C1CC1 ; CCc1c(-c2ccc(NC(=O)C(NC(=O)OC(C)(C)C)C(C3CC3)C3CC3)nc2)c(C)nn1COCC[Si](C)(C)C]

K₂CO₃ (1.45M aq. solution, 0.651 mL, 0.94 mmol) was added to a solution of the product from Preparation 4 (200 mg, 0.47 mmol) and the product from Preparation 6 (172 mg, 0.47 mmol) in DMF (2 mL) in a microwave vial. The reaction mixture was degassed and purged with nitrogen for 10 minutes. Pd(dppf)Cl₂·DCM (82.0 mg, 0.14 mmol) was added, the vial was sealed and the reaction mixture was shaken at 90° C. for 3.5 hours. The reaction mixture was cooled, filtered through a PTFE filter and purified directly by prep. acidic HPLC, to afford the title compound (82 mg, 30% yield). LCMS (METHOD 3) (ES): m/z 584.2 [M−H]⁻, RT=1.06 min.

### Preparation 8: 2-amino-3,3-dicyclopropyl-N-[5-[5-ethyl-3-methyl-1-(2-trimethylsilylethoxymethyl)pyrazol-4-yl]-2-pyridyl]propenamide hydrochloride

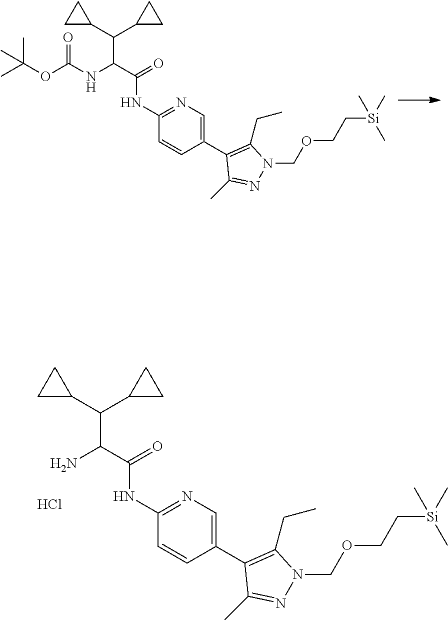 [SMILES: CCc1c(-c2ccc(NC(=O)C(NC(=O)OC(C)(C)C)C(C3CC3)C3CC3)nc2)c(C)nn1COCC[Si](C)(C)C ; CCc1c(-c2ccc(NC(=O)C(N)C(C3CC3)C3CC3)nc2)c(C)nn1COCC[Si](C)(C)C ; Cl]

Hydrogen chloride (4M solution in 1,4-dioxane, 1.0 mL, 4.0 mmol) was added to a solution of the product from Preparation 7 (80.0 mg, 0.137 mmol) in MeOH (2 mL) and stirred at room temperature for 2 hours. The reaction mixture was diluted with MeOH (5 mL) and concentrated in vacuo to afford the title compound (68 mg, assume 100% yield). LCMS (METHOD 3) (ES): m/z 484.3 [M−H]⁻, RT=0.74 min.

### Preparation 9: ethyl 2-(3-methoxypropyl)pyrazole-3-carboxylate

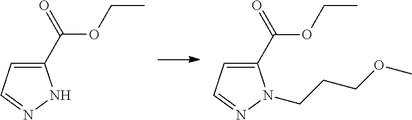

DEAD (40% solution in toluene, 0.91 mL, 4.64 mmol) was added slowly to a solution of ethyl 1H-pyrazole-5-carboxylate (500 mg, 3.57 mmol), 3-methoxypropan-1-ol (0.41 mL, 4.28 mmol) and triphenylphosphine (1.20 g, 4.64 mmol) in anhydrous THF (12 mL) at 0° C. The reaction mixture was stirred at room temperature for 18 hours. The reaction mixture was concentrated in vacuo and the obtained crude compound was purified by silica column chromatography (230-400 mesh), eluting with EtOAc in heptane, to afford the title compound as a colourless oil (3.95 g, 77% yield). 1H NMR (400 MHz, CDCl₃) δ 7.48 (d, J=2.0 Hz, 1H), 6.83 (d, J=2.0 Hz, 1H), 4.66 (t, J=7.0 Hz, 2H), 4.34 (q, J=7.1 Hz, 2H), 3.38 (t, J=6.2 Hz, 2H), 3.32 (s, 3H), 2.16-2.02 (m, 2H), 1.38 (t, J=7.1 Hz, 3H).

### Preparation 10: 2-(3-methoxypropyl)pyrazole-3-carboxylic acid

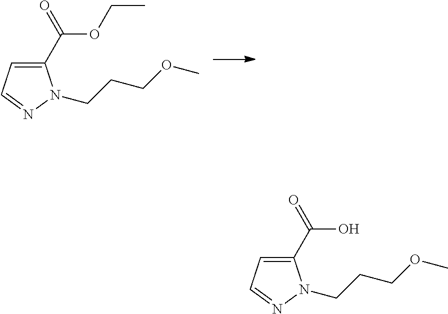

A solution of LiOH (202 mg, 8.44 mmol) in water (7 mL) was added to a solution of the product from Preparation 9 (597 mg, 2.81 mmol) in MeOH (14 mL) and stirred at room temperature for 1.5 hours. The pH was adjusted to -3 with hydrogen chloride (5M aq. solution) and extracted with DCM (3×10 mL). The combined organic layers were dried over Na₂SO₄, filtered and concentrated in vacuo to leave the title compound (540 mg, assume 100% yield). 1H NMR (600 MHz, CDCl₃) δ 7.54 (d, J=2.0 Hz, 1H), 6.96 (d, J=2.0 Hz, 1H), 4.69 (t, J=7.0 Hz, 2H), 3.41 (t, J=6.2 Hz, 2H), 3.33 (s, 3H), 2.14 (ddd, J=13.2, 7.1, 6.2 Hz, 2H).

### Preparation 11: N-[1-(dicyclopropylmethyl)-2-[[5-[5-ethyl-3-methyl-1-(2-trimethylsilylethoxymethyl)pyrazol-4-yl]-2-pyridyl]amino]-2-oxo-ethyl]-2-(3-methoxypropyl)pyrazole-3-carboxamide

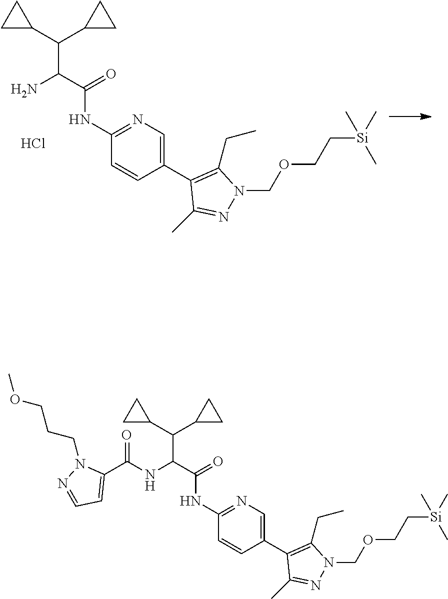

HATU (17.0 mg, 0.045 mmol) was added to a solution of the product from Preparation 8 (22.0 mg, 0.045 mmol), the product from Preparation 10 (8.3 mg, 0.045 mmol) and DIPEA (0.031 mL, 0.18 mmol) in DMF (1 mL) and the reaction mixture was stirred at room temperature for 30 minutes. The crude reaction mixture was purified directly by acidic prep. HPLC to afford the title compound (19 mg, 70% yield). LCMS (METHOD 3) (ES): m/z 650.3 [M+H]⁺, RT=0.99 min.

### Preparation 12: N-[1-(dicyclopropylmethyl)-2-[[5-[5-ethyl-3-methyl-1-(2-trimethylsilylethoxymethyl)pyrazol-4-yl]-2-pyridyl]amino]-2-oxo-ethyl]-2-isopropyl-pyrazole-3-carboxamide

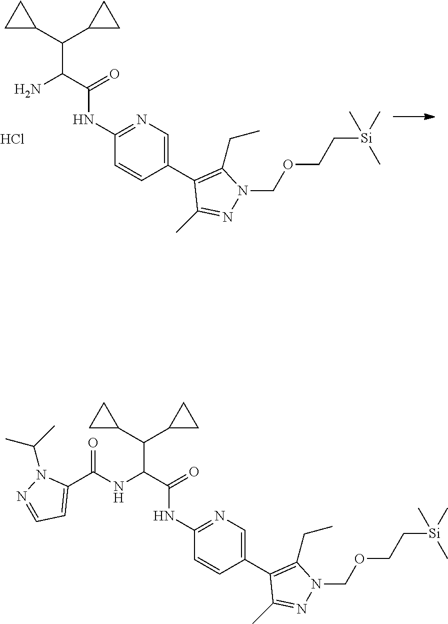

According to the method of Preparation 11 the compound of Preparation 8 (22 mg, 0.045 mmol) was reacted with 2-isopropylpyrazole-3-carboxylic acid (6.9 mg, 0.045 mmol) to give the title compound as an off-white solid (18 mg, 69% yield). LCMS (METHOD 3) (ES): m/z 620.3 [M+H]⁺, RT=1.02 min.

### Preparation 13: N-[1-(dicyclopropylmethyl)-2-[[5-[5-ethyl-3-methyl-1-(2-trimethylsilylethoxymethyl)pyrazol-4-yl]-2-pyridyl]amino]-2-oxo-ethyl]-2-(2-methoxyethyl)pyrazole-3-carboxamide

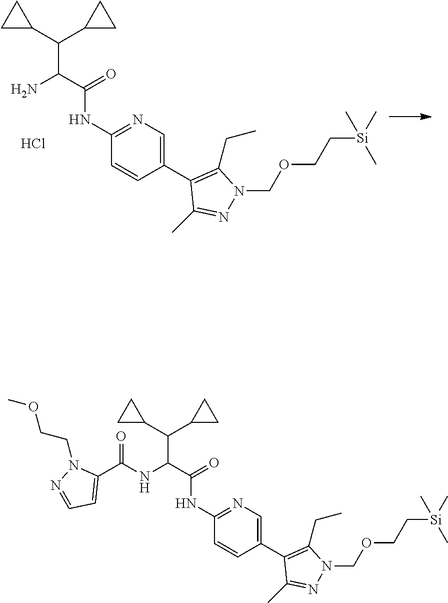

According to the method of Preparation 11 the compound of Preparation 8 (22 mg, 0.045 mmol) was reacted with 2-(2-methoxyethyl)pyrazole-3-carboxylic acid (7.7 mg, 0.045 mmol) to give the title compound as an off-white solid (19 mg, 71% yield). LCMS (METHOD 3) (ES): m/z 634.4 [M+H]⁺, RT=0.97 min.

### Preparation 14: ethyl 2-(tert-butoxycarbonylamino)-3,3-dicyclopropyl-propanoate

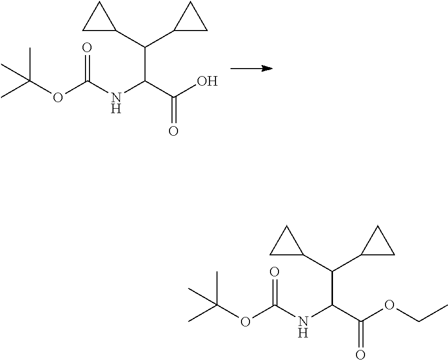 [SMILES: CC(C)(C)OC(=O)NC(C(=O)O)C(C1CC1)C1CC1 ; CCOC(=O)C(NC(=O)OC(C)(C)C)C(C1CC1)C1CC1]

Cs₂CO₃ (7.46 g, 22.9 mmol) was added to a solution of the product from Preparation 3 (5.14 g, 19.1 mmol) and stirred at room temperature for 30 minutes. Ethyl iodide (2.30 mL, 28.6 mmol) was added and the reaction mixture was stirred at 50° C. for 3 hours. The cooled reaction mixture was diluted with water (200 mL) and extracted with Et₂O (2×60 mL). The combined organic layer was dried over Na₂SO₄, filtered and concentrated in vacuo, to afford the title compound as a pale yellow oil (5.61 g, 98% yield). 1H NMR (400 MHz, CDCl₃) δ 5.25 (d, J=9.3 Hz, 1H), 4.49 (dd, J=9.4, 3.7 Hz, 1H), 4.30-4.10 (m, 2H), 1.45 (s, 9H), 1.28 (t, J=7.1 Hz, 3H), 0.87-0.60 (m, 3H), 0.60-0.29 (m, 4H), 0.33-0.04 (m, 4H).

### Preparation 15: ethyl 2-amino-3,3-dicyclopropyl-propanoate hydrochloride

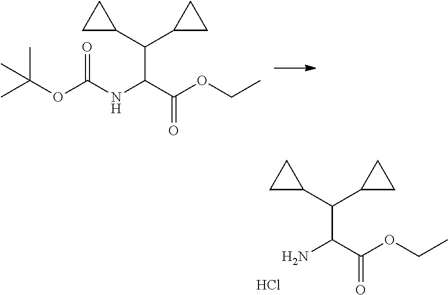 [SMILES: CCOC(=O)C(NC(=O)OC(C)(C)C)C(C1CC1)C1CC1 ; Cl ; CCOC(=O)C(N)C(C1CC1)C1CC1]

Acetyl chloride (5 mL) was added to ethanol (40 mL) dropwise at 0° C. On complete addition the solution was stirred at 0° C. for 15 minutes then warmed to room temperature over 30 mins. The product from Preparation 14 (4.0 g, 13.4 mmol) was added and the reaction mixture was stirred for 1 hour. The solution was concentrated in vacuo to afford the title compound (3.1 g, assume 100% yield) that was used without purification. LCMS (METHOD 3) (ES): m/z 198.2 [M+H]⁺, RT=0.50 min.

### Preparation 16: ethyl 3,3-dicyclopropyl-2-[(2-ethylpyrazole-3-carbonyl)amino]propanoate

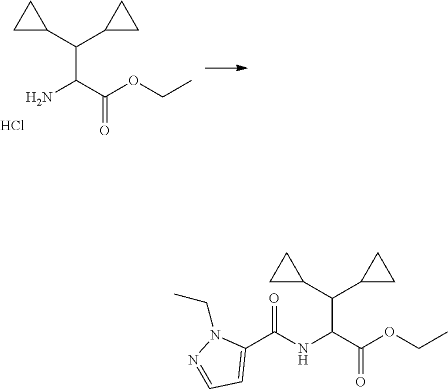 [SMILES: Cl ; CCOC(=O)C(N)C(C1CC1)C1CC1 ; CCOC(=O)C(NC(=O)c1ccnn1CC)C(C1CC1)C1CC1]

HATU (4.82 g, 12.7 mmol) was added to a solution of the product from Preparation 15 (2.47 g, 10.6 mmol), 2-ethylpyrazole-3-carboxylic acid (1.48 g, 10.6 mmol) and DIPEA (7.36 mL, 42.3 mmol) in MeCN (25 mL) and stirred at room temperature for 2 hours. The reaction mixture was concentrated in vacuo to low volume and diluted with water (200 mL). The solution was extracted with EtOAc (2×50 mL) and the combined extracts were dried over Na₂SO₄, filtered and concentrated in vacuo. The obtained crude compound was purified by silica column chromatography (230-400 mesh), eluting with EtOAc in heptane, to afford the title compound as a pale yellow oil (2.63 g, 78% yield). 1H NMR (600 MHz, CDCl₃) δ 7.48 (d, J=2.0 Hz, 1H), 6.73 (d, J=8.7 Hz, 1H), 6.57 (d, J=2.0 Hz, 1H), 4.94 (dd, J=8.7, 3.0 Hz, 1H), 4.59 (dtt, J=20.5, 13.3, 7.2 Hz, 2H), 4.36-4.12 (m, 2H), 1.44 (t, J=7.2 Hz, 3H), 1.31 (t, J=7.1 Hz, 3H), 0.83-0.69 (m, 3H), 0.66-0.41 (m, 4H), 0.38-0.17 (m, 4H).

### Preparation 17: 2-[[5-ethyl-3-methyl-4-(6-nitro-3-pyridyl)pyrazol-1-yl]methoxy]ethyl-trimethyl-silane

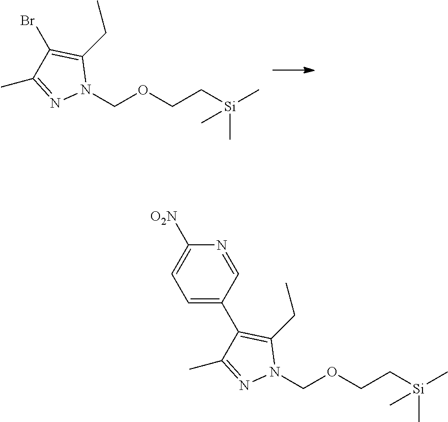

K₂CO₃ (1.45M aq. solution, 2.16 mL, 3.13 mmol) was added to a solution of the product from Preparation 5 (500 mg, 1.57 mmol) and 2-nitro-5-(4,4,5,5-tetramethyl-1,3,2-dioxaborolan-2-yl)pyridine (392 mg, 1.57 mmol) in DMF (6 mL) in a 20 mL microwave vial. The reaction mixture was degassed and purged with nitrogen for 10 minutes. Pd(dppf)Cl₂·DCM (128 mg, 0.157 mmol) was added, the vial was capped and the reaction mixture was stirred at 90° C. for 18 hours. The cooled reaction mixture was diluted with water (30 mL) and EtOAc (50 mL), filtered through Celite™ and partitioned. The aqueous phase was washed with EtOAc (50 mL) and the combined organic phase was washed with water (20 mL), brine solution (20 mL) then dried over MgSO₄ and concentrated in vacuo. The obtained crude compound was purified by silica column chromatography (230-400 mesh), eluting with EtOAc in heptane, to afford the title compound (47 mg, 8.3% yield). 1H NMR (400 MHz, CDCl₃) δ 8.60-8.53 (m, 1H), 8.38-8.29 (m, 1H), 7.97-7.88 (m, 1H), 5.44 (d, J=0.9 Hz, 2H), 3.72-3.57 (m, 2H), 2.84-2.61 (m, 2H), 2.33 (d, J=37.0 Hz, 3H), 1.27-1.14 (m, 3H), 1.01-0.87 (m, 2H), 0.02-−0.02 (m, 9H). (approx. 6:1 ratio of regioisomers).

### Preparation 18: 5-[5-ethyl-3-methyl-1-(2-trimethylsilylethoxymethyl)pyrazol-4-yl]pyridin-2-amine

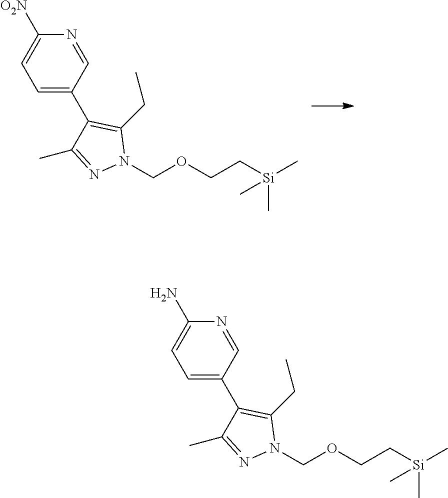 [SMILES: CCc1c(-c2ccc([N+](=O)[O-])nc2)c(C)nn1COCC[Si](C)(C)C ; CCc1c(-c2ccc(N)nc2)c(C)nn1COCC[Si](C)(C)C]

10% Pd/C (10 mg) was added to a solution of the product from Preparation 17 (47 mg, 0.13 mmol) in MeOH (3 mL). The flask was flushed with argon before the reaction mixture was stirred under hydrogen at atmospheric pressure at room temperature for 1 hour. The catalyst was filtered off and the filtrate was concentrated in vacuo to afford the title compound (35 mg, 81% yield). 1H NMR (400 MHz, CDCl₃) δ 8.01-7.93 (m, 1H), 7.37-7.30 (m, 1H), 6.60-6.51 (m, 1H), 5.39 (s, 2H), 4.51 (s, 2H), 3.71-3.55 (m, 2H), 2.74-2.51 (m, 2H), 2.33-2.12 (m, 3H), 1.23-1.05 (m, 3H), 1.00-0.83 (m, 2H), −0.00-−0.03 (m, 9H). (approx. 6:1 ratio of regioisomers).

### Preparation 19: N-[1-(dicyclopropylmethyl)-2-[[5-[5-ethyl-3-methyl-1-(2-trimethylsilylethoxymethyl)pyrazol-4-yl]-2-pyridyl]amino]-2-oxo-ethyl]-2-ethyl-pyrazole-3-carboxamide

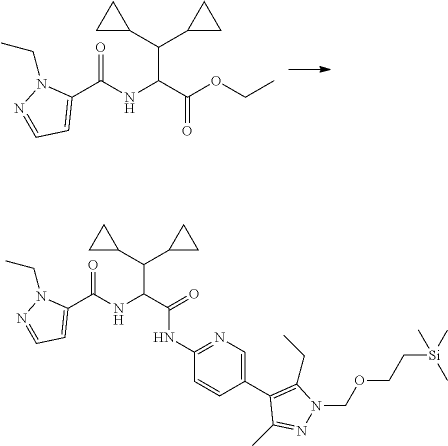

AlMe₃ (2M solution in toluene, 0.093 mL, 0.186 mmol) was added to a solution of the product from Preparation 16 (29.8 mg, 0.093 mmol) and the product from Preparation 18 (31.0 mg, 0.93 mmol) in a sealed 2 mL microwave vial, under constant argon stream. After initial gas evolution ceased, the reaction mixture was stirred at 100° C. for 3 hours. The cooled reaction mixture was carefully quenched with MeOH (2 mL) then filtered. The crude filtrate was purified by basic prep. HPLC to afford the title compound (6 mg, 10.6% yield). LCMS (METHOD 3) (ES): m/z 606.3 [M+H]⁺, RT=0.91 min.

### Preparation 20: N-[1-[(5-bromo-4-methoxy-2-pyridyl)carbamoyl]-2,2-dicyclopropyl-ethyl]-2-ethyl-pyrazole-3-carboxamide

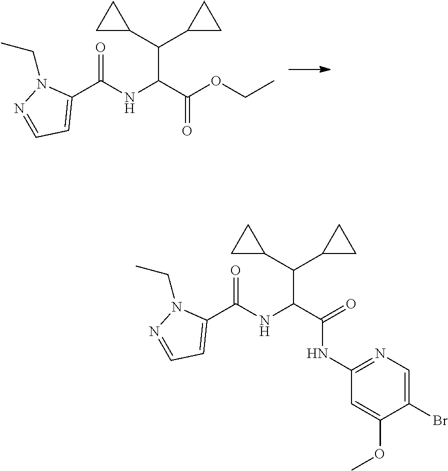

AlMe₃ (2M solution in toluene, 0.485 mL, 0.97 mmol) was added to a solution of the product from Preparation 16 (155 mg, 0.485 mmol) and 5-bromo-4-methoxy-2-pyridin-2-amine (108 mg, 0.534 mmol) in a sealed 2 mL microwave vial, under a constant argon stream. After initial gas evolution ceased, the reaction mixture was stirred at 90° C. for 3 hours. The cooled reaction mixture was carefully quenched into water (25 mL) and acidified to pH 4 with citric acid. The reaction mixture was extracted with EtOAc (2×50 mL). The combined organic extracts were dried over MgSO₄, filtered and concentrated in vacuo. The crude product was triturated with Et₂O, collected and dried to afford the title compound as a colourless solid (11.0 mg, 5% yield). LCMS (METHOD 3) (ES): m/z 478.1 [M+H]⁺, RT=0.80 min.

### Preparation 21: 2-[[3,5-dimethyl-4-(4,4,5,5-tetramethyl-1,3,2-dioxaborolan-2-yl)pyrazol-1-yl]methoxy]ethyl-trimethyl-silane

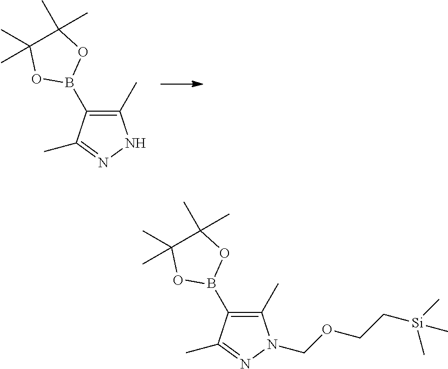

SEM chloride (5.78 mL, 32.6 mmol) was added to a solution of 3,5-dimethyl-4-(4,4,5,5-tetramethyl-1,3,2-dioxaborolan-2-yl)-1H-pyrazole (5.00 g, 22.5 mmol) and K₂CO₃ (6.22 g, 45.0 mmol) in NMP (34 mL) and stirred at room temperature for 18 hours. The reaction mixture was diluted with EtOAc (150 mL) and filtered to remove precipitate. The filtrate was successively washed with water (2×50 mL), saturated aq. NaHCO₃ (50 mL) and brine solution (50 mL), dried over MgSO₄, filtered and concentrated in vacuo. The obtained crude compound was purified by silica column chromatography (230-400 mesh), eluting with EtOAc (0-30%) in heptane, to afford the title compound as a colourless oil (5.85 g, 74% yield). 1H NMR (300 MHz, DMSO-d6) δ 5.27 (s, 2H), 3.60-3.41 (m, 2H), 2.36 (s, 3H), 2.17 (s, 3H), 1.25 (s, 12H), 0.97-0.71 (m, 2H), −0.05 (s, 9H).

### Preparation 22: N-[1-(dicyclopropylmethyl)-2-[[5-[3,5-dimethyl-1-(2-trimethylsilylethoxymethyl)pyrazol-4-yl]-4-methoxy-2-pyridyl]amino]-2-oxo-ethyl]-2-ethyl-pyrazole-3-carboxamide

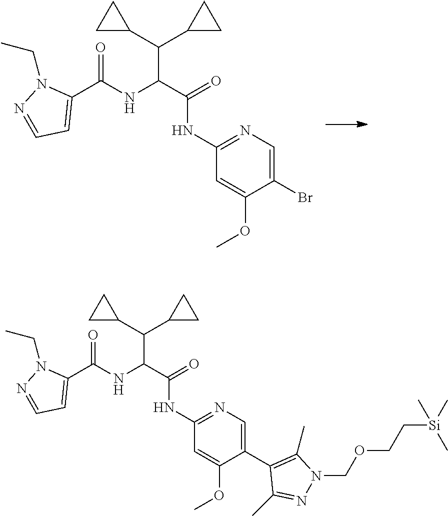 [SMILES: CCn1nccc1C(=O)NC(C(=O)Nc1cc(OC)c(Br)cn1)C(C1CC1)C1CC1 ; CCn1nccc1C(=O)NC(C(=O)Nc1cc(OC)c(-c2c(C)nn(COCC[Si](C)(C)C)c2C)cn1)C(C1CC1)C1CC1]

K₂CO₃ (7.98 mg, 0.058 mmol) was added to a solution of the product from Preparation 20 (11.0 mg, 0.023 mmol) and the product from Preparation 21 (8.54 mg, 0.024 mmol) in THF:H₂O (4:1, 10 mL) in a 20 mL microwave vial. The reaction mixture was degassed and purged with nitrogen for 10 minutes. Pd(dppf)Cl₂·DCM (0.85 mg, 0.001 mmol) was added, the vial was capped and the reaction mixture was stirred at 90° C. for 18 hours. The cooled reaction mixture was diluted with brine solution (10 mL). The aqueous phase was extracted with EtOAc (25 mL). The organic phase was dried over MgSO₄ and concentrated in vacuo. The obtained crude compound was purified by silica column chromatography (230-400 mesh), eluting with EtOAc in heptane, to afford the title compound (12.6 mg, 87% yield). LCMS (METHOD 3) (ES): m/z 622.3 [M+H]⁺, RT=0.92 min.

### Preparation 23: N-[1-[(6-bromo-2-fluoro-3-pyridyl)carbamoyl]-2,2-dicyclopropyl-ethyl]-2-ethyl-pyrazole-3-carboxamide

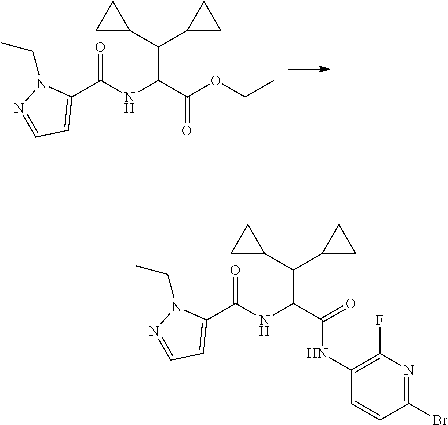

According to the method of Preparation 19 the compound of Preparation 16 (100 mg, 0.313 mmol) was reacted with 6-bromo-2-fluoro-pyridin-3-amine (65.8 mg, 0.344 mmol). The cooled reaction mixture was carefully quenched with MeOH:H₂O (5 mL, 4:1) then filtered. The crude filtrate was purified by basic prep. HPLC to afford the title compound (56 mg, 25% yield). 1H NMR (400 MHz, DMSO-d6) δ 10.33 (s, 1H), 8.49 (d, J=8.6 Hz, 1H), 8.41 (dd, J=9.8, 8.3 Hz, 1H), 7.62 (d, J=8.3 Hz, 1H), 7.48 (d, J=2.0 Hz, 1H), 6.99 (d, J=2.1 Hz, 1H), 4.98 (t, J=8.1 Hz, 1H), 4.47 (qd, J=7.1, 1.6 Hz, 2H), 1.28 (t, J=7.1 Hz, 3H), 1.00-0.69 (m, 3H), 0.52-0.05 (m, 8H).

### Preparation 24: N-[1-(dicyclopropylmethyl)-2-[[6-[3,5-dimethyl-1-(2-trimethylsilylethoxymethyl)pyrazol-4-yl]-2-fluoro-3-pyridyl]amino]-2-oxo-ethyl]-2-ethyl-pyrazole-3-carboxamide

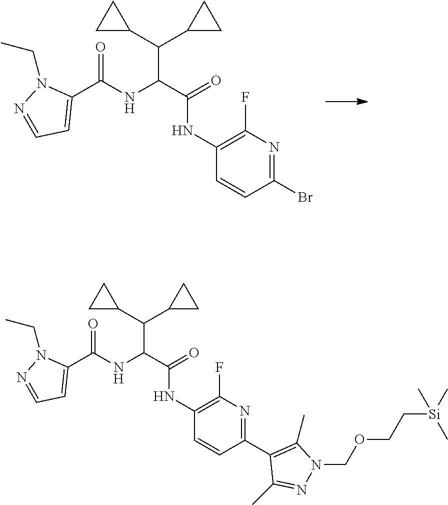 [SMILES: CCn1nccc1C(=O)NC(C(=O)Nc1ccc(Br)nc1F)C(C1CC1)C1CC1 ; CCn1nccc1C(=O)NC(C(=O)Nc1ccc(-c2c(C)nn(COCC[Si](C)(C)C)c2C)nc1F)C(C1CC1)C1CC1]

According to the method of Preparation 7 the compound of Preparation 23 (56.0 mg, 0.12 mmol) was reacted with the product from Preparation 21 (85.0 mg, 0.24 mmol). The crude filtrate was purified by acidic prep. HPLC to afford the title compound (17 mg, 23% yield). LCMS (METHOD 3) (ES): m/z 610.4 [M+H]⁺, RT=0.94 min.

### Preparation 25: N-[1-[(5-bromo-6-methoxy-2-pyridyl)carbamoyl]-2,2-dicyclopropyl-ethyl]-2-ethyl-pyrazole-3-carboxamide

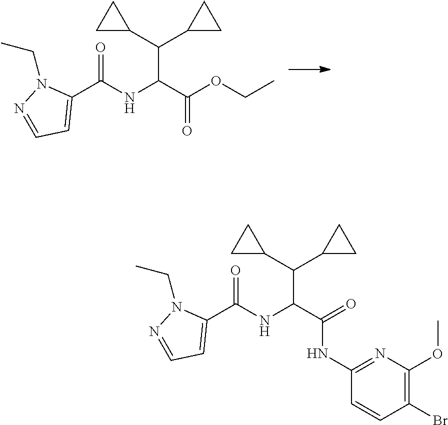

According to the method of Preparation 20 the compound of Preparation 16 (205 mg, 0.64 mmol) was reacted with 5-bromo-6-methoxy-pyridin-2-amine (143 mg, 0.71 mmol) to afford the title compound as a colourless solid (240 mg, 78% yield). LCMS (METHOD 3) (ES): m/z 478.1 [M+H]⁺, RT=0.96 min.

### Preparation 26: N-[1-(dicyclopropylmethyl)-2-[[5-[3,5-dimethyl-1-(2-trimethylsilylethoxymethyl)pyrazol-4-yl]-6-methoxy-2-pyridyl]amino]-2-oxo-ethyl]-2-ethyl-pyrazole-3-carboxamide

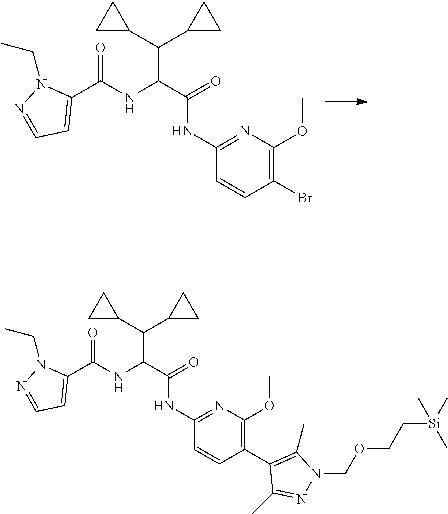

According to the method of Preparation 22 the compound of Preparation 25 (240 mg, 0.50 mmol) was reacted with the product from Preparation 21 (195 mg, 0.53 mmol) to afford the title compound as a colourless solid (258 mg, 82% yield). LCMS (METHOD 3) (ES): m/z 622.3 [M+H]⁺, RT=0.94 min.

### Preparation 27: N-[1-[(5-bromo-4-fluoro-2-pyridyl)carbamoyl]-2,2-dicyclopropyl-ethyl]-2-ethyl-pyrazole-3-carboxamide

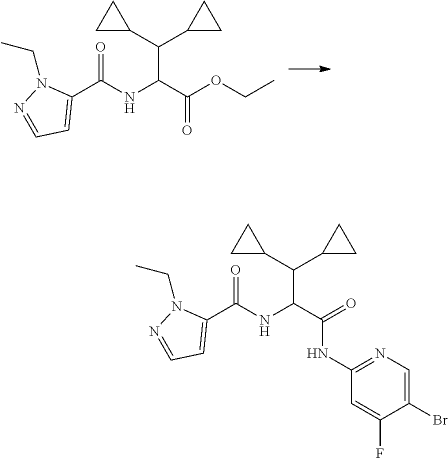

AlMe₃ (2M solution in toluene, 0.164 mL, 0.329 mmol) was added to a solution of 5-bromo-4-methoxy-2-pyridin-2-amine (62.8 mg, 0.329 mmol) in toluene (2 mL) in a sealed 5 mL microwave vial, under a constant argon stream. The reaction mixture was stirred for 3-4 minutes, vented to release pressure and a solution of the product from Preparation 16 (100 mg, 0.313 mmol) in toluene (1 mL) was added. The reaction mixture was stirred at 45° C. for 18 hours. The cooled reaction mixture was carefully quenched with citric acid (2% solution, 8 mL). The reaction mixture was extracted with EtOAc (2×15 mL). The combined organic extracts were dried over MgSO₄, filtered and concentrated in vacuo. The obtained crude compound was purified by silica column chromatography (230-400 mesh), eluting with EtOAc in heptane, to afford the title compound (66 mg, 45% yield). LCMS (METHOD 3) (ES): m/z 466.0 [M+H]⁺, RT=0.85 min.

### Preparation 28: N-[1-(dicyclopropylmethyl)-2-[[5-[3,5-dimethyl-1-(2-trimethylsilylethoxymethyl)pyrazol-4-yl]-4-fluoro-2-pyridyl]amino]-2-oxo-ethyl]-2-ethyl-pyrazole-3-carboxamide

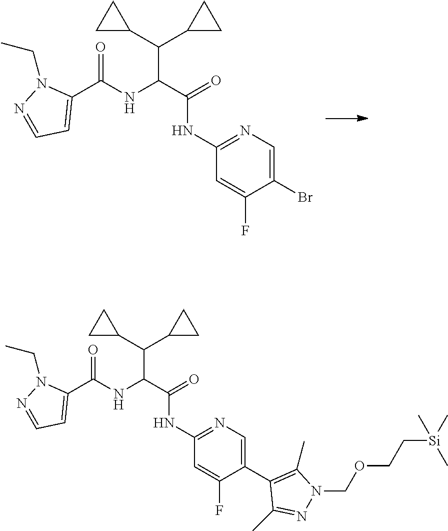 [SMILES: CCn1nccc1C(=O)NC(C(=O)Nc1cc(F)c(Br)cn1)C(C1CC1)C1CC1 ; CCn1nccc1C(=O)NC(C(=O)Nc1cc(F)c(-c2c(C)nn(COCC[Si](C)(C)C)c2C)cn1)C(C1CC1)C1CC1]

According to the method of Preparation 7 the compound of Preparation 27 (66.0 mg, 0.12 mmol) was reacted with the product from Preparation 21 (85.0 mg, 0.24 mmol). The title compound so obtained was progressed without purification (86 mg, assume 100% yield). LCMS (METHOD 3) (ES): m/z 610.3 [M+H]⁺, RT=0.98 min.

### Preparation 29: N-[1-[(5-bromo-3-fluoro-2-pyridyl)carbamoyl]-2,2-dicyclopropyl-ethyl]-2-ethyl-pyrazole-3-carboxamide

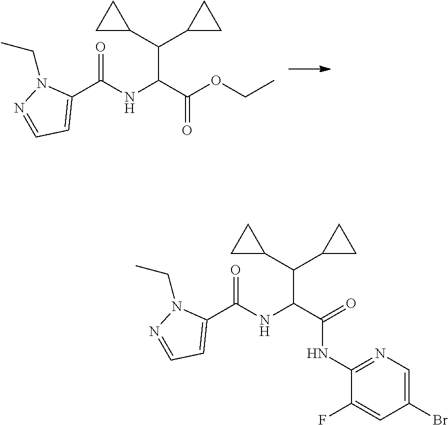

According to the method of Preparation 20 the compound of Preparation 16 (290 mg, 0.91 mmol) was reacted with 5-bromo-3-fluoro-pyridin-2-amine (190 mg, 0.99 mmol) to afford the title compound as an orange solid (90 mg, 21% yield). LCMS (METHOD 3) (ES): m/z 465.9 [M+H]⁺, RT=0.77 min.

### Preparation 30: N-[1-(dicyclopropylmethyl)-2-[[5-[3,5-dimethyl-1-(2-trimethylsilylethoxymethyl)pyrazol-4-yl]-3-fluoro-2-pyridyl]amino]-2-oxo-ethyl]-2-ethyl-pyrazole-3-carboxamide

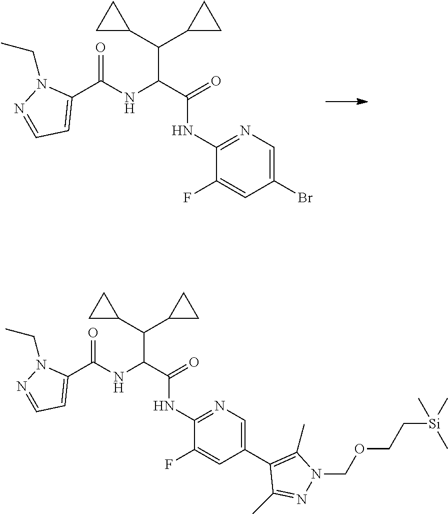 [SMILES: CCn1nccc1C(=O)NC(C(=O)Nc1ncc(Br)cc1F)C(C1CC1)C1CC1 ; CCn1nccc1C(=O)NC(C(=O)Nc1ncc(-c2c(C)nn(COCC[Si](C)(C)C)c2C)cc1F)C(C1CC1)C1CC1]

According to the method of Preparation 22 the compound of Preparation 29 (90 mg, 0.19 mmol) was reacted with the product from Preparation 21 (78.6 mg, 0.21 mmol) to afford the title compound as a colourless solid (115 mg, assume 100%% yield). LCMS (METHOD 3) (ES): m/z 610.4 [M+H]⁺, RT=0.91 min.

### Preparation 31: N-[1-[(5-bromo-6-methyl-2-pyridyl)carbamoyl]-2,2-dicyclopropyl-ethyl]-2-ethyl-pyrazole-3-carboxamide

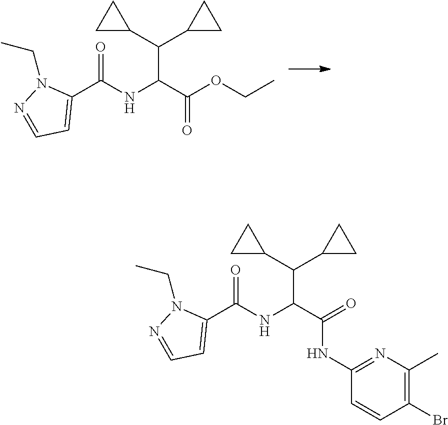

According to the method of Preparation 20 the compound of Preparation 16 (145 mg, 0.454 mmol) was reacted with 5.bromo-6-methyl-pyridin-2-amine (93.5 mg, 0.50 mmol) to afford the title compound as a colourless solid (165 mg, 79% yield). LCMS (METHOD 3) (ES): m/z 462.2 [M+H]⁺, RT=0.98 min.

### Preparation 32: N-[1-(dicyclopropylmethyl)-2-[[5-[3,5-dimethyl-1-(2-trimethylsilylethoxymethyl)pyrazol-4-yl]-6-methyl-2-pyridyl]amino]-2-oxo-ethyl]-2-ethyl-pyrazole-3-carboxamide

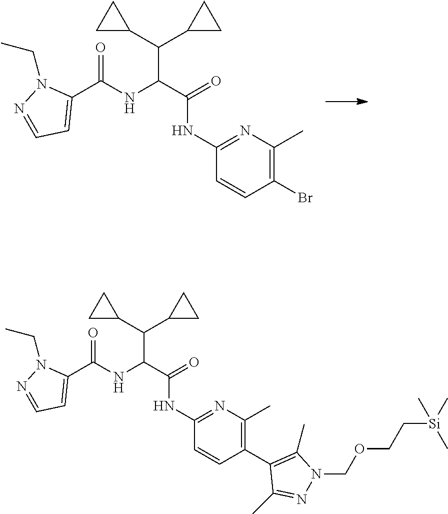

According to the method of Preparation 22 the compound of Preparation 31 (129 mg, 0.28 mmol) was reacted with the product from Preparation 21 (128 mg, 0.364 mmol) to afford the title compound as a colourless solid (170 mg, assume 100%% yield). LCMS (METHOD 3) (ES): m/z 606.4 [M+H]⁺, RT=0.97 min.

### Preparation 33: N-[1-[(5-bromo-3-methoxy-2-pyridyl)carbamoyl]-2,2-dicyclopropyl-ethyl]-2-ethyl-pyrazole-3-carboxamide

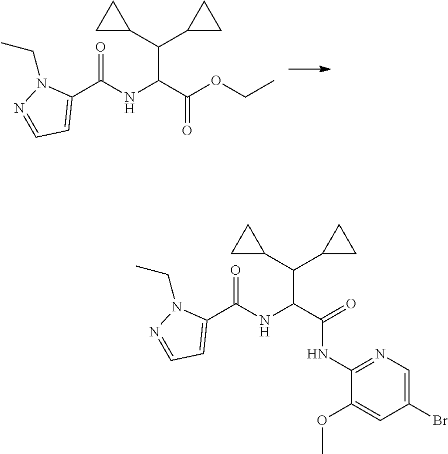

According to the method of Preparation 27 the compound of Preparation 16 (100 mg, 0.313 mmol) was reacted with 5-bromo-6-methyl-pyridin-2-amine (66.8 mg, 0.329 mmol) to afford the title compound as a colourless solid (32 mg, 21% yield). LCMS (METHOD 3) (ES): m/z 476.1 [M+H]⁺, RT=0.74 min.

### Preparation 34: N-[1-(dicyclopropylmethyl)-2-[[5-[3,5-dimethyl-1-(2-trimethylsilylethoxymethyl)pyrazol-4-yl]-3-methoxy-2-pyridyl]amino]-2-oxo-ethyl]-2-ethyl-pyrazole-3-carboxamide

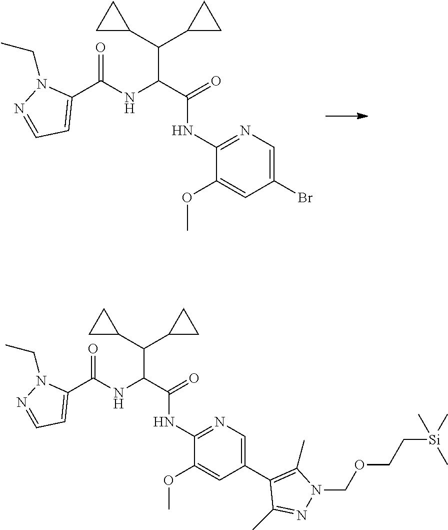 [SMILES: CCn1nccc1C(=O)NC(C(=O)Nc1ncc(Br)cc1OC)C(C1CC1)C1CC1 ; CCn1nccc1C(=O)NC(C(=O)Nc1ncc(-c2c(C)nn(COCC[Si](C)(C)C)c2C)cc1OC)C(C1CC1)C1CC1]

According to the method of Preparation 22 the compound of Preparation 33 (32 mg, 0.067 mmol) was reacted with the product from Preparation 21 (26 mg, 0.074 mmol) to afford the title compound as a colourless solid (22 mg, 52% yield). LCMS (METHOD 3) (ES): m/z 623.5 [M+H]⁺, RT=0.90 min.

### Preparation 35: (4-methoxyphenyl)methyl (2R)-2-(tert-butoxycarbonylamino)-3,3-dicyclopropyl-propanoate and (4-methoxyphenyl)methyl (2S)-2-(tert-butoxycarbonylamino)-3,3-dicyclopropyl-propanoate

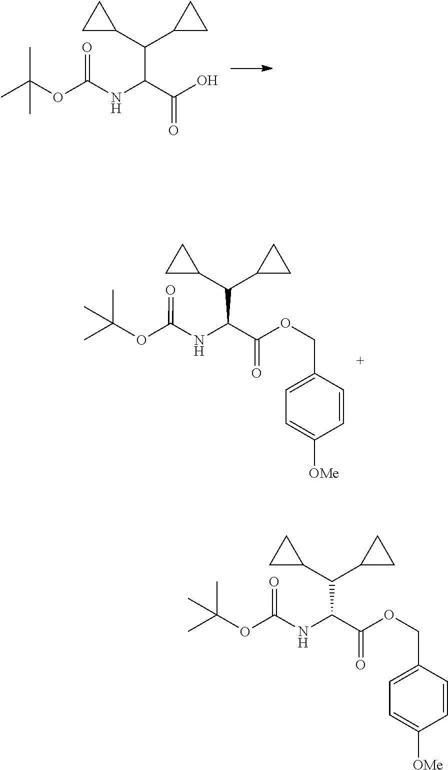 [SMILES: CC(C)(C)OC(=O)NC(C(=O)O)C(C1CC1)C1CC1 ; COc1ccc(COC(=O)[C@@H](NC(=O)OC(C)(C)C)C(C2CC2)C2CC2)cc1 ; COc1ccc(COC(=O)[C@H](NC(=O)OC(C)(C)C)C(C2CC2)C2CC2)cc1]

EDC (7.77 g, 40.5 mmol) was added to a mixture of the acid of Preparation 3 (7.28 g, 27.0 mmol), 4-methoxybenzylalcohol (4.48 g, 32.4 mmol) and DMAP (3.3 g, 27.0 mmol) in DCM (100 mL) and stirred overnight at room temperature. The reaction mixture was washed with 0.25M HCl (15 mL), dried (Na₂SO₄) and evaporated. Purification by column chromatography (silica, eluting with EtOAc:heptane) gave the racemic title compound (9.30 g, 88%) as a white solid. 1H NMR (300 MHz, CDCl₃) δ 7.38-7.18 (m, 2H), 6.98-6.79 (m, 2H), 5.24 (d, J=9.3 Hz, 1H), 5.09 (s, 2H), 4.53 (d, J=9.3 Hz, 1H), 3.81 (s, 3H), 1.44 (s, 9H), 0.80-0.55 (m, 3H), 0.55-0.26 (m, 4H), 0.25-0.10 (m, 3H), 0.07-−0.05 (m, 1H); LCMS (METHOD 3) (ES): m/z 390.3 [M+H]⁺, RT=0.95 min. The two enantiomers were separated by preparative chiral SFC giving (4-methoxyphenyl)methyl (2R)-2-(tert-butoxycarbonylamino)-3,3-dicyclopropyl-propanoate (Preparation 35a) (Column: Lux A2 (4.6 mm×250 mm, 5 μm), Eluent: 20:80 IPA:CO₂ (0.2% v/v NH₃), Temp: 40° C., Flow rate: 4 mL/min, BPR: 125 Bar, retention time: 1.4 min) and (4-methoxyphenyl)methyl (2S)-2-(tert-butoxycarbonylamino)-3,3-dicyclopropyl-propanoate (Preparation 35b) (Column: Lux A2 (4.6 mm×250 mm, 5 μm), Eluent: 20:80 IPA:CO₂ (0.2% v/v NH₃), Temp: 40° C., Flow rate: 4 mL/min, BPR: 125 Bar, retention time: 1.9 min).

### Preparation 36: (2S)-2-(Tert-butoxycarbonylamino)-3,3-dicyclopropyl-propanoic acid

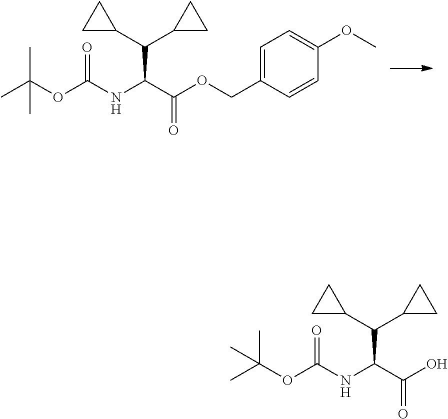

A solution of (4-methoxyphenyl)methyl (2S)-2-(tert-butoxycarbonylamino)-3,3-dicyclopropyl-propanoate (Preparation 35b) (5.30 g, 13.6 mmol) in MeOH (25 mL) was hydrogenated over 10% Pd/C (250 mg) using a hydrogen balloon. After 2½ hours the reaction mixture was filtered and evaporated. Purification by column chromatography (silica, eluting with EtOAc:heptane) gave the title compound (3.50 g, 96%) as a clear syrup. 1H NMR (400 MHz, DMSO-d6) Mixture of rotamers δ 12.41 (s, 1H), 6.81 (d, J=9.0 Hz, 0.82H), 6.48 (d, J=8.2 Hz, 0.18H), 4.12 (dd, J=9.0, 4.4 Hz, 0.82H), 4.05 (s, 0.18H), 1.39 (s, 7.4H), 1.25 (s, 1.6H), 1.02-0.88 (m, 1H), 0.83-0.72 (m, 1H), 0.56-0.42 (m, 2H), 0.41-0.20 (m, 4H), 0.19-0.01 (m, 3H); LCMS (METHOD 3) (ES): m/z 268.4 [M−H]⁻, RT=0.71 min.

### Preparation 37: ethyl (2S)-2-amino-3,3-dicyclopropyl-propanoate hydrochloride

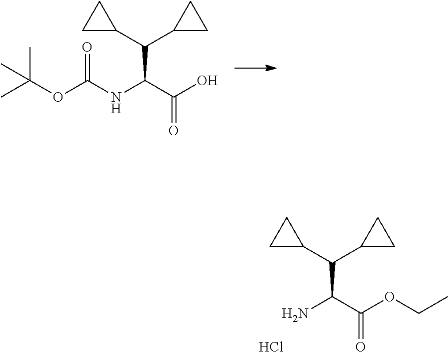 [SMILES: CC(C)(C)OC(=O)N[C@H](C(=O)O)C(C1CC1)C1CC1 ; Cl ; CCOC(=O)[C@@H](N)C(C1CC1)C1CC1]

Hydrogen chloride (2M in EtOH, 80 mL) was added to a solution of the product from Preparation 36 (2.4 g, 8.1 mmol) in DCM (80 mL) and the reaction mixture was stirred at room temperature for 2 hours then concentrated in vacuo to leave the title compound as a colourless solid (1.88 g, 100% yield). Used without purification.

### Preparation 38: methyl (2S)-3,3-dicyclopropyl-2-[(2-isopropylpyrazole-3-carbonyl)amino]propanoate

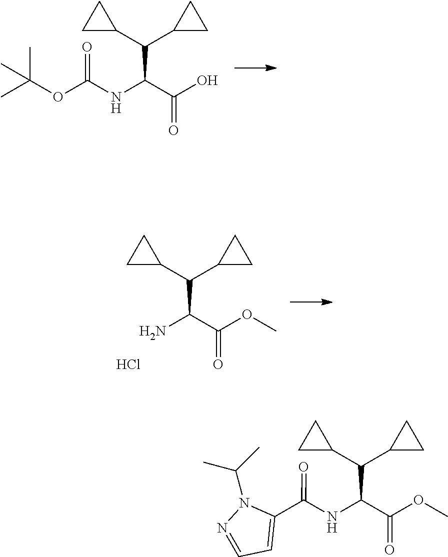 [SMILES: CC(C)(C)OC(=O)N[C@H](C(=O)O)C(C1CC1)C1CC1 ; Cl ; COC(=O)[C@@H](N)C(C1CC1)C1CC1 ; COC(=O)[C@@H](NC(=O)c1ccnn1C(C)C)C(C1CC1)C1CC1]

The product of Preparation 36 (2.10 g, 7.80 mmol) was dissolved in hydrogen chloride (2M solution in MeOH, 80 mL) and stirred at room temperature for 18 hours. The reaction mixture was concentrated in vacuo to afford the intermediate compound methyl (2S)-2-amino-3,3-dicyclopropyl-propanoate hydrochloride (1.71 g, 7.78 mmol). HATU (1.20 g, 3.16 mmol) was added to a solution of methyl (2S)-2-amino-3,3-dicyclopropyl-propanoate hydrochloride (1.71 g, 7.78 mmol), 2-isopropylpyrazole-3-carboxylic acid (1.32 g, 8.56 mmol) and DIPEA (4.07 mL, 23.3 mmol) in MeCN (30 mL) and stirred at room temperature for 16 hours. The reaction mixture was concentrated in vacuo to low volume and diluted with water (200 mL). The solution was extracted with EtOAc (2×50 mL) and the combined extracts were dried over Na₂SO₄, filtered and concentrated in vacuo. The obtained crude compound was purified by silica column chromatography (230-400 mesh), eluting with EtOAc in heptane, to afford the title compound as a colourless oil (786 mg, 32% yield). 1H NMR (600 MHz, CDCl₃) δ 7.51 (d, J=1.9 Hz, 1H), 6.72 (d, J=8.7 Hz, 1H), 6.54 (d, J=2.0 Hz, 1H), 5.46 (hept, J=6.6 Hz, 1H), 4.96 (dd, J=8.6, 3.0 Hz, 1H), 3.78 (s, 3H), 1.49 (dd, J=15.2, 6.6 Hz, 6H), 0.75 (dddd, J=20.2, 9.5, 5.5, 2.8 Hz, 3H), 0.66-0.41 (m, 4H), 0.37-0.14 (m, 4H); LCMS (METHOD 3) (ES): m/z 320.2 [M+H]⁺, RT=0.78 min.

### Preparation 39: 5-[5-ethyl-3-methyl-1-(2-trimethylsilylethoxymethyl)pyrazol-4-yl]-6-fluoro-pyridin-2-amine

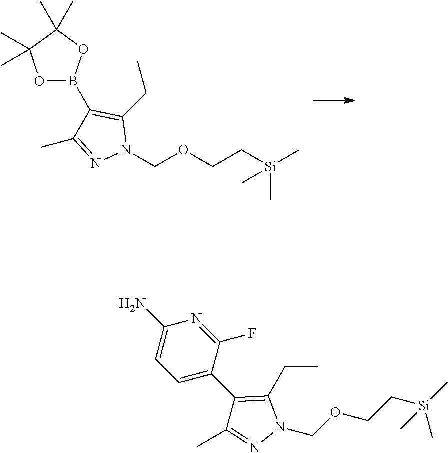

According to the method of Preparation 22 the compound of Preparation 6 (10.4 g, 28.4 mmol) was reacted with 5-bromo-6-fluoro-pyridin-2-amine (4.97 g, 26.0 mmol) to afford the title compound as a colourless solid (6.30 g, 69% yield). LCMS (METHOD 3) (ES): m/z 351.2 [M+H]⁺, RT=0.84 min.

### Preparation 40: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-isopropyl-pyrazole-3-carboxamide

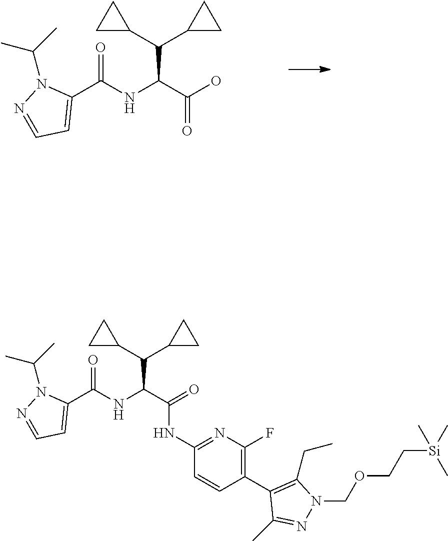 [SMILES: CC(C)n1nccc1C(=O)N[C@H](C(=O)O)C(C1CC1)C1CC1 ; CCc1c(-c2ccc(NC(=O)[C@@H](NC(=O)c3ccnn3C(C)C)C(C3CC3)C3CC3)nc2F)c(C)nn1COCC[Si](C)(C)C]

According to the method of Preparation 27 the compound of Preparation 38 (96.0 mg, 0.301 mmol) was reacted with the product from Preparation 39 (100 mg, 0.285 mmol) to afford the title compound as a colourless oil (151 mg, 79% yield). LCMS (METHOD 3) (ES): m/z 638.4 [M+H]⁺, RT=1.03 min.

### Preparation 41: 5-[3,5-dimethyl-1-(2-trimethylsilylethoxymethyl)pyrazol-4-yl]-6-fluoro-pyridin-2-amine

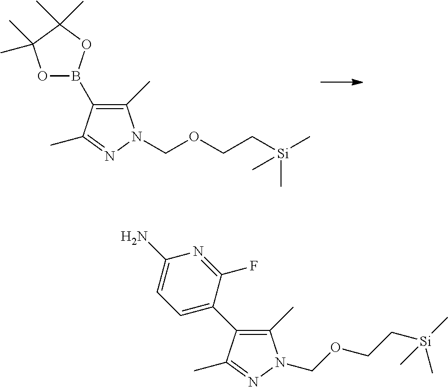

According to the method of Preparation 22 the compound of Preparation 21 (1.50 g, 4.26 mmol) was reacted with 5-bromo-6-fluoro-pyridin-2-amine (0.78 g, 4.10 mmol) to afford the title compound as a pale yellow solid (1.36 g, 99% yield). LCMS (METHOD 3) (ES): m/z 337.2 [M+H]⁺, RT=0.80 min.

### Preparation 42: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-isopropyl-pyrazole-3-carboxamide

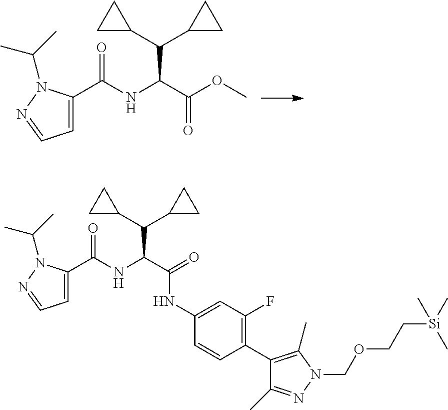 [SMILES: COC(=O)[C@@H](NC(=O)c1ccnn1C(C)C)C(C1CC1)C1CC1 ; Cc1nn(COCC[Si](C)(C)C)c(C)c1-c1ccc(NC(=O)[C@@H](NC(=O)c2ccnn2C(C)C)C(C2CC2)C2CC2)cc1F]

According to the method of Preparation 27 the product of Preparation 38 (96.0 mg, 0.301 mmol) was reacted with the product from Preparation 41 (100 mg, 0.297 mmol) to afford the crude title compound as a yellow oil (187 mg, assume 100% yield) that was used without further purification. LCMS (METHOD 3) (ES): m/z 624.4 [M+H]⁺, RT=1.00 min.

### Preparation 43: ethyl (2S)-2-(tert-butoxycarbonylamino)-3,3-dicyclopropyl-propanoate

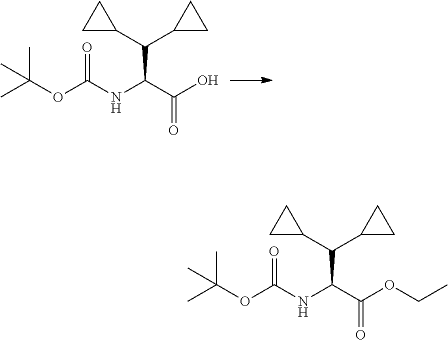 [SMILES: CC(C)(C)OC(=O)N[C@H](C(=O)O)C(C1CC1)C1CC1 ; CCOC(=O)[C@@H](NC(=O)OC(C)(C)C)C(C1CC1)C1CC1]

EDC (5.30 g, 28.0 mmol) was added to a solution of the product from Preparation 36 (5.0 g, 19.0 mmol), DMAP (0.45 g, 3.7 mmol), EtOH (3.2 mL) in DCM (25 mL) and stirred at room temperature for 18 hours. The reaction mixture was washed with KHSO₄ (1M aq. solution, 20 mL), dried over Na₂SO₄, filtered and concentrated in vacuo to afford the title compound as a clear thick oil (5.41 g, 98% yield). 1H NMR (600 MHz, CDCl₃) δ 5.25 (d, J=9.3 Hz, 1H), 4.49 (dd, J=9.4, 3.8 Hz, 1H), 4.26-4.07 (m, 2H), 1.45 (s, 9H), 1.28 (t, J=7.1 Hz, 3H), 0.82-0.58 (m, 3H), 0.58-0.34 (m, 4H), 0.32-0.07 (m, 4H).

### Preparation 44: ethyl (2S)-3,3-dicyclopropyl-2-[(2-ethylpyrazole-3-carbonyl)amino]propanoate

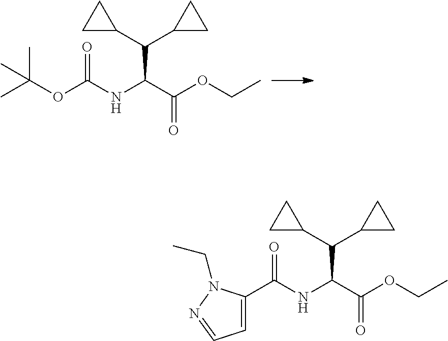 [SMILES: CCOC(=O)[C@@H](NC(=O)OC(C)(C)C)C(C1CC1)C1CC1 ; CCOC(=O)[C@@H](NC(=O)c1ccnn1CC)C(C1CC1)C1CC1]

According to the method of Preparation 38 the product of Preparation 43 (5.41 g, 18.2 mmol) was initially reacted with 2M HCl in EtOH (20 mL) and subsequently with 2-ethylpyrazole-3-carboxylic acid (2.80 g, 20 mmol) to afford the crude title compound as a colourless oil (4.64 g, 80% yield). LCMS (METHOD 3) (ES): m/z 320.2 [M+H]⁺, RT=0.75 min.

### Preparation 45: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-ethyl-pyrazole-3-carboxamide

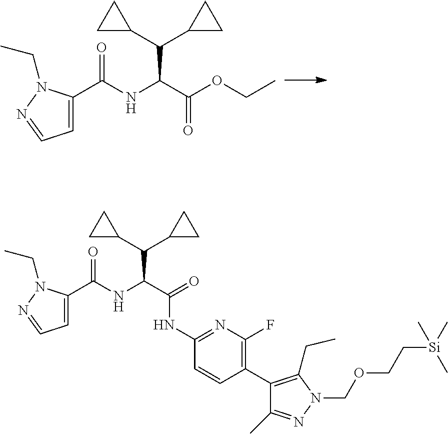

According to the method of Preparation 27 the product of Preparation 44 (142.0 mg, 0.445 mmol) was reacted with the product from Preparation 39 (163.6 mg, 0.467 mmol) to afford the crude title compound as a yellow oil (218 mg, 78% yield) that was used without further purification. LCMS (METHOD 3) (ES): m/z 624.4 [M+H]⁺, RT=1.01 min.

### Preparation 46: N-[1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-ethyl-pyrazole-3-carboxamide

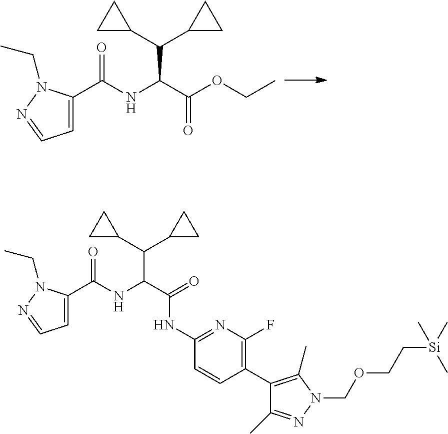

According to the method of Preparation 20 the product of Preparation 44 (191.0 mg, 0.60 mmol) was reacted with the product from Preparation 41 (211.3 mg, 0.623 mmol) to afford the crude title compound as a yellow oil (364 mg, assume 100% yield) that was used without further purification.

### Preparation 47: 6-[3,5-dimethyl-1-(2-trimethylsilylethoxymethyl)pyrazol-4-yl]-5-fluoro-pyridin-3-amine

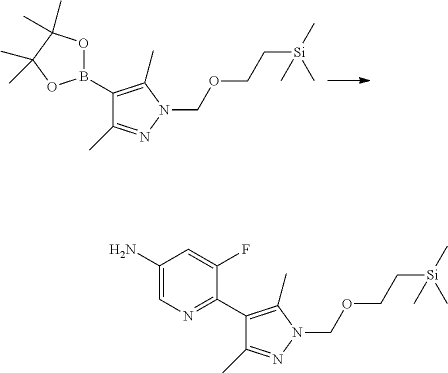

According to the method of Preparation 17 the compound of Preparation 21 (194.5 mg, 0.524 mmol) was reacted with 6-bromo-5-fluoro-pyridin-3-amine (100 mg, 0.524 mmol) to afford the title compound (125 mg, 71% yield). 1H NMR (600 MHz, DMSO-d6) δ 7.86 (t, J=2.0 Hz, 1H), 6.79 (dd, J=12.4, 2.2 Hz, 1H), 5.65 (s, 2H), 5.33 (s, 2H), 3.58-3.47 (m, 2H), 2.17 (s, 3H), 2.05 (s, 3H), 0.91-0.71 (m, 2H), −0.04 (s, 9H); LCMS (METHOD 3) (ES): m/z 338.1 [M+H]⁺, RT=0.75 min.

### Preparation 48: N-[1-(dicyclopropylmethyl)-2-[[6-[3,5-dimethyl-1-(2-trimethylsilylethoxymethyl)pyrazol-4-yl]-5-fluoro-3-pyridyl]amino]-2-oxo-ethyl]-2-ethyl-pyrazole-3-carboxamide

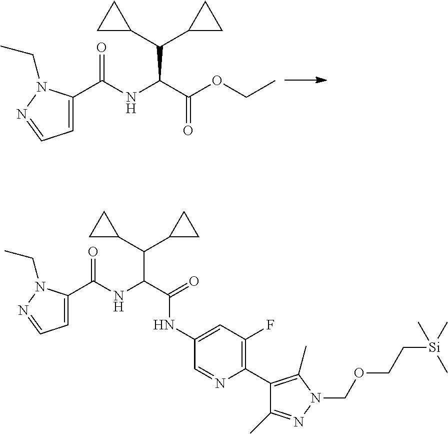

According to the method of Preparation 27 the product of Preparation 44 (45.0 mg, 0.141 mmol) was reacted with the product from Preparation 47 (50 mg, 0.148 mmol) to afford the crude title compound (29 mg, 34% yield). 1H NMR (400 MHz, DMSO-d6) δ 10.73 (s, 1H), 8.64 (t, J=1.7 Hz, 1H), 8.55 (d, J=8.6 Hz, 1H), 8.13 (dd, J=12.1, 2.1 Hz, 1H), 7.49 (d, J=2.0 Hz, 1H), 7.02 (d, J=2.0 Hz, 1H), 5.37 (s, 2H), 4.82 (t, J=8.0 Hz, 1H), 4.56-4.40 (m, 2H), 3.63-3.46 (m, 2H), 2.24 (d, J=1.4 Hz, 3H), 2.11 (d, J=1.2 Hz, 3H), 1.29 (t, J=7.1 Hz, 3H), 1.00-0.76 (m, 5H), 0.54-0.07 (m, 8H), -0.04 (s, 9H); LCMS (METHOD 3) (ES): m/z 610.3 [M+H]⁺, RT=0.94 min.

### Preparation 49: ethyl (2S)-3,3-dicyclopropyl-2-[(3-methylisoxazole-4-carbonyl)amino]propanoate

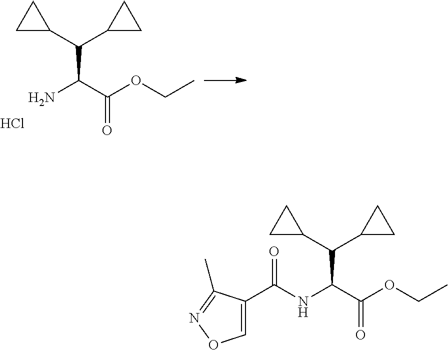 [SMILES: Cl ; CCOC(=O)[C@@H](N)C(C1CC1)C1CC1 ; CCOC(=O)[C@@H](NC(=O)c1conc1C)C(C1CC1)C1CC1]

HATU (162.7 mg, 0.428 mmol) was added to a solution of the product from Preparation 37 (100 mg, 0.428 mmol), 3-methylisoxazole-4-carboxylic acid (54.4 mg, 0.428 mmol) and DIPEA (0.169 mL, 0.856 mmol) in DMF (1 mL) and stirred at room temperature for 1 hour. The reaction mixture was purified directly by acidic prep. HPLC to afford the title compound (103 mg, 78% yield); LCMS (METHOD 3) (ES): m/z 305.2 [M−H]⁻, RT=0.75 min.

### Preparation 50: ethyl (2S)-3,3-dicyclopropyl-2-[(3-ethylisoxazole-4-carbonyl)amino]propanoate

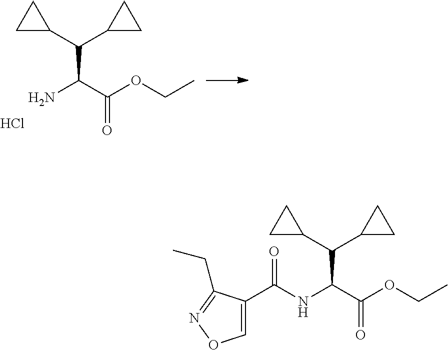 [SMILES: Cl ; CCOC(=O)[C@@H](N)C(C1CC1)C1CC1 ; CCOC(=O)[C@@H](NC(=O)c1conc1CC)C(C1CC1)C1CC1]

HATU (162.7 mg, 0.428 mmol) was added to a solution of the product from Preparation 37 (100 mg, 0.428 mmol), 3-ethylisoxazole-4-carboxylic acid (60.4 mg, 0.428 mmol) and DIPEA (0.169 mL, 0.856 mmol) in DMF (1 mL) and stirred at room temperature for 1 hour. The reaction mixture was purified directly by acidic prep. HPLC to afford the title compound (99 mg, 72% yield); LCMS (METHOD 3) (ES): m/z 321.8 [M+H]⁺, RT=0.80 min.

### Preparation 51: ethyl (2S)-3,3-dicyclopropyl-2-[(3-isopropylisoxazole-4-carbonyl)amino]propanoate

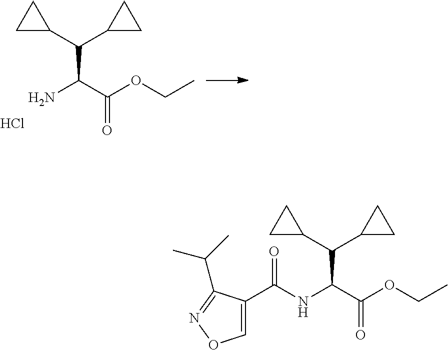 [SMILES: Cl ; CCOC(=O)[C@@H](N)C(C1CC1)C1CC1 ; CCOC(=O)[C@@H](NC(=O)c1conc1C(C)C)C(C1CC1)C1CC1]

HATU (112.7 mg, 0.297 mmol) was added to a solution of the product from Preparation 37 (69.3 mg, 0.297 mmol), 3-isopropylisoxazole-4-carboxylic acid (46.0 mg, 0.297 mmol) and DIPEA (0.103 mL, 0.593 mmol) in DMF (1 mL) and stirred at room temperature for 1 hour. The reaction mixture was purified directly by acidic prep. HPLC to afford the title compound (103 mg, 78% yield); LCMS (METHOD 3) (ES): m/z 333.3 [M−H]⁻, RT=0.84 min.

### Preparation 52: N-[1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-3-methyl-isoxazole-4-carboxamide

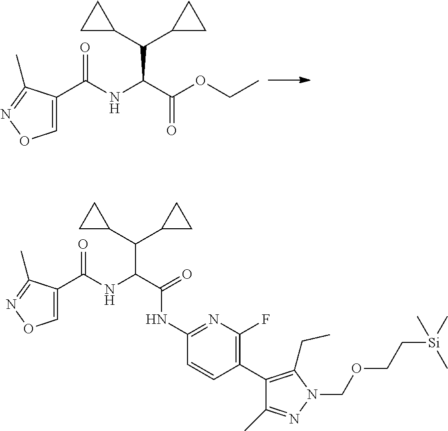 [SMILES: CCOC(=O)[C@@H](NC(=O)c1conc1C)C(C1CC1)C1CC1 ; CCc1c(-c2ccc(NC(=O)C(NC(=O)c3conc3C)C(C3CC3)C3CC3)nc2F)c(C)nn1COCC[Si](C)(C)C]

According to the method of Preparation 27 the product of Preparation 49 (50 mg, 0.163 mmol) was reacted with the product from Preparation 39 (60 mg, 0.171 mmol) to afford the title compound after prep. acidic HPLC (60 mg, 60% yield); LCMS (METHOD 3) (ES): m/z 611.3 [M+H]⁺, RT=0.98 min.

### Preparation 53: N-[1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-3-ethyl-isoxazole-4-carboxamide

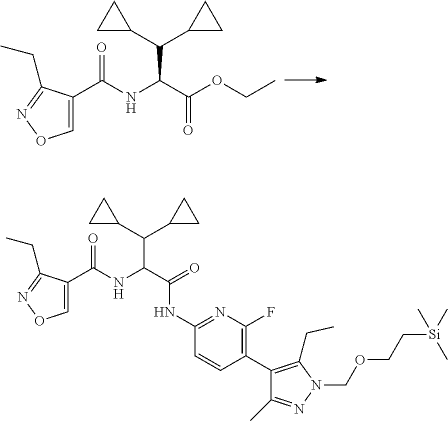

According to the method of Preparation 27 the product of Preparation 50 (48 mg, 0.153 mmol) was reacted with the product from Preparation 39 (56.3 mg, 0.161 mmol) to afford the title compound after prep. acidic HPLC (51 mg, 53% yield); LCMS (METHOD 3) (ES): m/z 625.3 [M+H]⁺, RT=1.00 min.

### Preparation 54: N-[1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-3-isopropyl-isoxazole-4-carboxamide

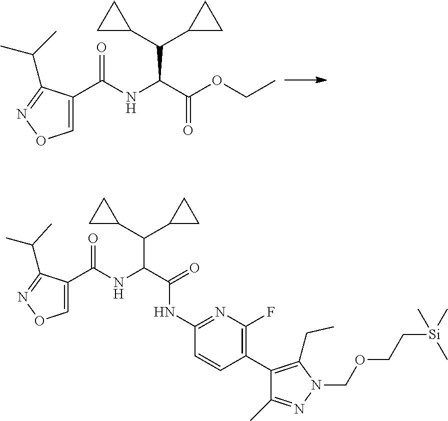

According to the method of Preparation 27 the product of Preparation 51 (36 mg, 0.108 mmol) was reacted with the product from Preparation 39 (39.6 mg, 0.113 mmol) to afford the title compound after prep. acidic HPLC (24 mg, 35% yield); LCMS (METHOD 3) (ES): m/z 639.4 [M+H]⁺, RT=1.03 min.

### Preparation 55: tert-butyl N-[(1S)-1-[(5-bromopyrazin-2-yl)carbamoyl]-2,2-dicyclopropyl-ethyl]carbamate

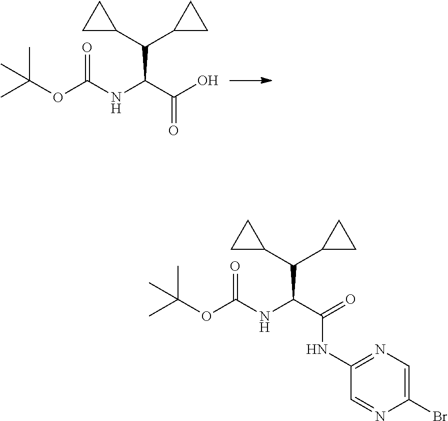 [SMILES: CC(C)(C)OC(=O)N[C@H](C(=O)O)C(C1CC1)C1CC1 ; CC(C)(C)OC(=O)N[C@H](C(=O)Nc1cnc(Br)cn1)C(C1CC1)C1CC1]

DIPEA (0.129 mL, 0.743 mmol) was added to a solution of the product from Preparation 36 (200 mg, 0.743 mmol) and HATU (282.4 mg, 0.743 mmol) in DMF (2 mL) in an argon flushed sealed vial and the reaction mixture was stirred at room temperature for 40 mins. Sodium hydride (60%, 99 mg, 2.23 mmol) was added to another vial, that was sealed and flushed with argon. A solution of 5-bromopyrazin-2-amine (388 mg, 2.23 mmol) in DMF (2 mL) was added slowly at 0° C. This was stirred for 1 hour at 0° C., then added carefully to the first vial at room temperature and the whole reaction mixture was stirred for 30 minutes. MeOH (1 mL) was added and the reaction mixture was filtered through a PTFE filter and the filtrate was purified directly by prep. basic HPLC to afford the title compound (118 mg, 37% yield). 1H NMR (400 MHz, DMSO-d6) δ 10.97 (s, 1H), 9.12 (s, 1H), 8.61 (d, J=1.4 Hz, 1H), 7.40-6.43 (m, 1H), 4.63-4.25 (m, 1H), 1.52-1.21 (m, 9H), 1.09-0.02 (m, 11H); LCMS (METHOD 3) (ES): m/z 425.3 [M−H]⁻, RT=0.86 min.

### Preparation 56: tert-butyl N-[(1S)-1-[(5-bromopyrimidin-2-yl)carbamoyl]-2,2-dicyclopropyl-ethyl]carbamate

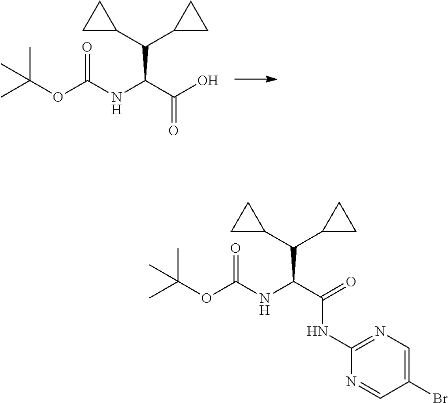 [SMILES: CC(C)(C)OC(=O)N[C@H](C(=O)O)C(C1CC1)C1CC1 ; CC(C)(C)OC(=O)N[C@H](C(=O)Nc1ncc(Br)cn1)C(C1CC1)C1CC1]

According to the method of Preparation 55 the product of Preparation 36 (600 mg, 2.23 mmol) was reacted with 5-bromopyrimidin-2-amine (387 mg, 2.23 mmol) to afford the title compound after prep. basic HPLC (198 mg, 62% yield). 1H NMR (400 MHz, DMSO-d6) δ 10.73 (s, 1H), 8.81 (s, 2H), 6.97-6.44 (m, 1H), 4.80-4.25 (m, 1H), 1.38 (d, J=7.1 Hz, 9H), 1.02-0.07 (m, 11H); LCMS (METHOD 3) (ES): m/z 423.3 [M−H]⁻, RT=0.76 min.

### Preparation 57: tert-butyl N-[(1S)-1-[(6-bromopyridazin-3-yl)carbamoyl]-2,2-dicyclopropyl-ethyl]carbamate

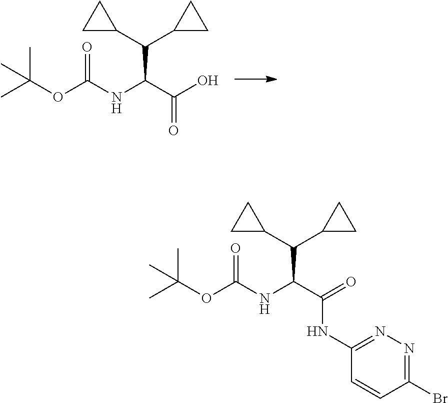 [SMILES: CC(C)(C)OC(=O)N[C@H](C(=O)O)C(C1CC1)C1CC1 ; CC(C)(C)OC(=O)N[C@H](C(=O)Nc1ccc(Br)nn1)C(C1CC1)C1CC1]

According to the method of Preparation 55 the product of Preparation 36 (600 mg, 2.23 mmol) was reacted with 6-bromopyridazin-3-amine (387 mg, 2.23 mmol) to afford the title compound after prep. basic HPLC (198 mg, 62% yield). 1H NMR (400 MHz, DMSO-d6) δ 11.28 (s, 1H), 8.27 (d, J=9.4 Hz, 1H), 7.97 (d, J=9.4 Hz, 1H), 7.30-6.93 (m, 1H), 4.83-4.09 (m, 1H), 1.50-1.28 (m, 9H), 1.02-0.02 (m, 11H); LCMS (METHOD 3) (ES): m/z 423.3 [M−H]⁻, RT=0.81 min.

### Preparation 58: tert-butyl N-[(1S)-1-[(2-bromopyrimidin-5-yl)carbamoyl]-2,2-dicyclopropyl-ethyl]carbamate

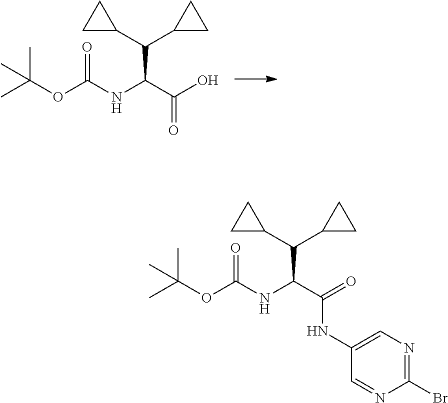 [SMILES: CC(C)(C)OC(=O)N[C@H](C(=O)O)C(C1CC1)C1CC1 ; CC(C)(C)OC(=O)N[C@H](C(=O)Nc1cnc(Br)nc1)C(C1CC1)C1CC1]

HATU (141.2 mg, 0.391 mmol) was added to a solution of the product from Preparation 36 (100 mg, 0.371 mmol), 2-bromopyrimidin-5-amine (71.1 mg, 0.407 mmol) and DIPEA (0.19 mL, 1.11 mmol) in DMF (1 mL) and stirred at room temperature for 1 hour. The reaction mixture was purified directly by basic prep. HPLC to afford the title compound (20 mg, 13% yield). 1H NMR (400 MHz, DMSO-d6) δ 10.60-10.30 (m, 1H), 8.90 (s, 2H), 7.02 (d, J=8.7 Hz, 1H), 4.43-4.08 (m, 1H), 1.40 (s, 9H), 0.98-0.03 (m, 11H); LCMS (METHOD 3) (ES): m/z 423.2 [M−H]⁻, RT=0.81 min.

### Preparation 59: tert-butyl N-[(1S)-1-[(6-bromo-5-methyl-3-pyridyl)carbamoyl]-2,2-dicyclopropyl-ethyl]carbamate

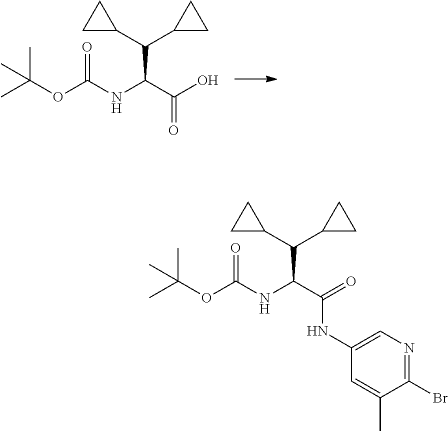 [SMILES: CC(C)(C)OC(=O)N[C@H](C(=O)O)C(C1CC1)C1CC1 ; Cc1cc(NC(=O)[C@@H](NC(=O)OC(C)(C)C)C(C2CC2)C2CC2)cnc1Br]

HATU (141.2 mg, 0.391 mmol) was added to a solution of the product from Preparation 36 (100 mg, 0.371 mmol), 6-bromo-5-methyl-pyridin-3-amine (76.4 mg, 0.407 mmol) and DIPEA (0.19 mL, 1.11 mmol) in DMF (1 mL) and stirred at room temperature for 1 hour. The reaction mixture was purified directly by basic prep. HPLC to afford the title compound (82 mg, 50% yield). 1H NMR (400 MHz, DMSO-d6) δ 10.30-9.94 (m, 1H), 8.43 (d, J=2.6 Hz, 1H), 7.98 (dd, J=2.7, 0.8 Hz, 1H), 7.06-6.45 (m, 1H), 4.41-4.03 (m, 1H), 2.32 (t, J=0.6 Hz, 3H), 1.48-1.29 (m, 9H), 0.99-0.03 (m, 11H); LCMS (METHOD 3) (ES): m/z 438.2 [M−H]⁻, RT=0.87 min.

### Preparation 60: tert-butyl N-[(1S)-1-[(6-bromo-5-methoxy-3-pyridyl)carbamoyl]-2,2-dicyclopropyl-ethyl]carbamate

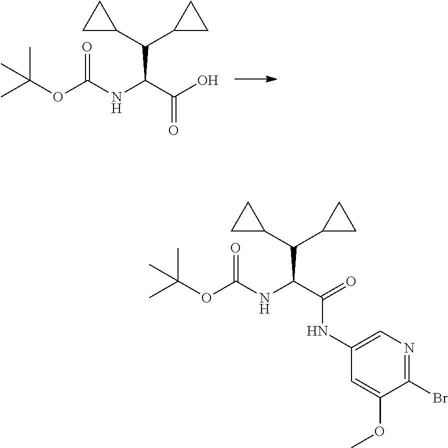 [SMILES: CC(C)(C)OC(=O)N[C@H](C(=O)O)C(C1CC1)C1CC1 ; COc1cc(NC(=O)[C@@H](NC(=O)OC(C)(C)C)C(C2CC2)C2CC2)cnc1Br]

HATU (141.2 mg, 0.391 mmol) was added to a solution of the product from Preparation 36 (100 mg, 0.371 mmol), 6-bromo-5-methoxy-pyridin-3-amine (82.9 mg, 0.407 mmol) and DIPEA (0.19 mL, 1.11 mmol) in DMF (1 mL) and stirred at room temperature for 1 hour. The reaction mixture was purified directly by basic prep. HPLC to afford the title compound (82 mg, 50% yield). 1H NMR (400 MHz, DMSO-d6) δ 10.42-9.90 (m, 1H), 8.24 (d, J=2.1 Hz, 1H), 7.75 (d, J=2.2 Hz, 1H), 7.11-6.46 (m, 1H), 4.44-4.03 (m, 1H), 3.86 (s, 3H), 1.58-1.26 (m, 9H), 1.08-0.03 (m, 11H); LCMS (METHOD 3) (ES): m/z 454.3 [M−H]⁻, RT=0.84 min.

### Preparation 61: tert-butyl N-[(1S)-1-(dicyclopropylmethyl)-2-[[5-(3,5-dimethyl-1H-pyrazol-4-yl)pyrazin-2-yl]amino]-2-oxo-ethyl]carbamate

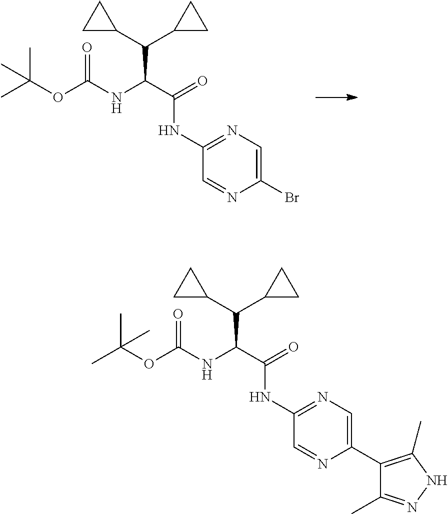 [SMILES: CC(C)(C)OC(=O)N[C@H](C(=O)Nc1cnc(Br)cn1)C(C1CC1)C1CC1 ; Cc1n[nH]c(C)c1-c1cnc(NC(=O)[C@@H](NC(=O)OC(C)(C)C)C(C2CC2)C2CC2)cn1]

According to the method of Preparation 7 the compound of Preparation 55 (50 mg, 0.118 mmol) was reacted with 3,5-dimethyl-4-(4,4,5,5-tetramethyl-1,3,2-dioxaborolan-2-yl)-1H-pyrazole (78.3 mg, 0.353 mmol). The crude filtrate was purified by acidic prep. HPLC to afford the title compound (39 mg, 75% yield). LCMS (METHOD 3) (ES): m/z 441.3 [M+H]⁺, RT=0.74 min.

### Preparation 62: tert-butyl N-[(1S)-1-(dicyclopropylmethyl)-2-[[5-(3,5-dimethyl-1H-pyrazol-4-yl)pyrimidin-2-yl]amino]-2-oxo-ethyl]carbamate

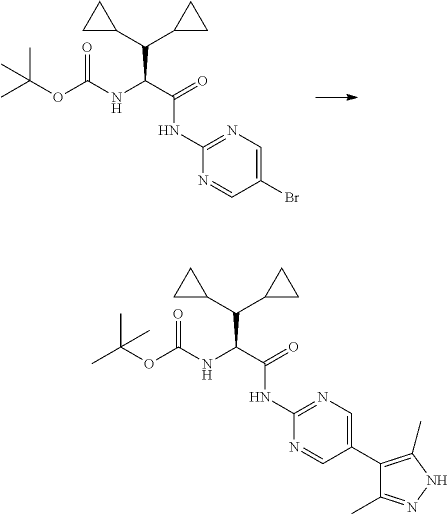 [SMILES: CC(C)(C)OC(=O)N[C@H](C(=O)Nc1ncc(Br)cn1)C(C1CC1)C1CC1 ; Cc1n[nH]c(C)c1-c1cnc(NC(=O)[C@@H](NC(=O)OC(C)(C)C)C(C2CC2)C2CC2)nc1]

According to the method of Preparation 7 the compound of Preparation 56 (50 mg, 0.118 mmol) was reacted with 3,5-dimethyl-4-(4,4,5,5-tetramethyl-1,3,2-dioxaborolan-2-yl)-1H-pyrazole (78.3 mg, 0.353 mmol). The crude filtrate was purified by acidic prep. HPLC to afford the title compound (52 mg, 100% yield). LCMS (METHOD 3) (ES): m/z 441.3 [M+H]⁺, RT=0.68 min.

### Preparation 63: isopropyl N-[(1S)-1-(dicyclopropylmethyl)-2-[[6-(3,5-dimethyl-1H-pyrazol-4-yl)pyridazin-3-yl]amino]-2-oxo-ethyl]carbamate

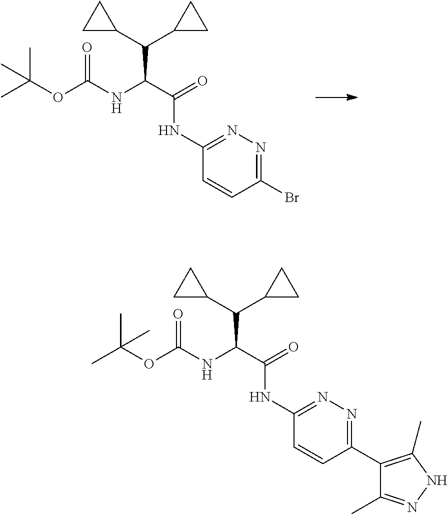 [SMILES: CC(C)(C)OC(=O)N[C@H](C(=O)Nc1ccc(Br)nn1)C(C1CC1)C1CC1 ; Cc1n[nH]c(C)c1-c1ccc(NC(=O)[C@@H](NC(=O)OC(C)(C)C)C(C2CC2)C2CC2)nn1]

According to the method of Preparation 7 the compound of Preparation 57 (50 mg, 0.118 mmol) was reacted with 3,5-dimethyl-4-(4,4,5,5-tetramethyl-1,3,2-dioxaborolan-2-yl)-1H-pyrazole (78.3 mg, 0.353 mmol). The crude filtrate was purified by acidic prep. HPLC to afford the title compound (28 mg, 54% yield). LCMS (METHOD 3) (ES): m/z 441.3 [M+H]⁺, RT=0.72 min.

### Preparation 64: tert-butyl N-[(1S)-1-(dicyclopropylmethyl)-2-[[2-(3,5-dimethyl-1H-pyrazol-4-yl)pyrimidin-5-yl]amino]-2-oxo-ethyl]carbamate

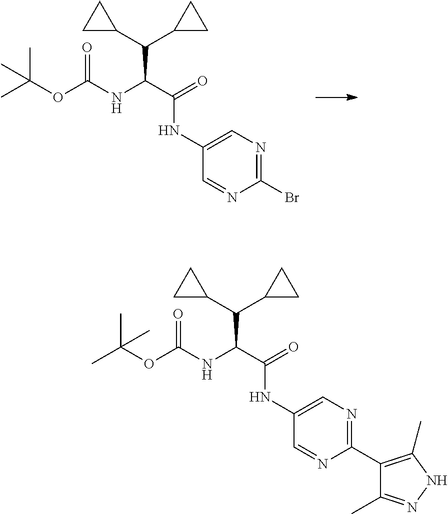 [SMILES: CC(C)(C)OC(=O)N[C@H](C(=O)Nc1cnc(Br)nc1)C(C1CC1)C1CC1 ; Cc1n[nH]c(C)c1-c1ncc(NC(=O)[C@@H](NC(=O)OC(C)(C)C)C(C2CC2)C2CC2)cn1]

According to the method of Preparation 7 the compound of Preparation 58 (20 mg, 0.047 mmol) was reacted with 3,5-dimethyl-4-(4,4,5,5-tetramethyl-1,3,2-dioxaborolan-2-yl)-1H-pyrazole (31.3 mg, 0.141 mmol). The crude filtrate was purified by acidic prep. HPLC to afford the title compound (11 mg, 53% yield). LCMS (METHOD 3) (ES): m/z 441.3 [M+H]⁺, RT=0.74 min.

### Preparation 65: tert-butyl N-[(1S)-1-(dicyclopropylmethyl)-2-[[6-(3,5-dimethyl-1H-pyrazol-4-yl)-5-methyl-3-pyridyl]amino]-2-oxo-ethyl]carbamate

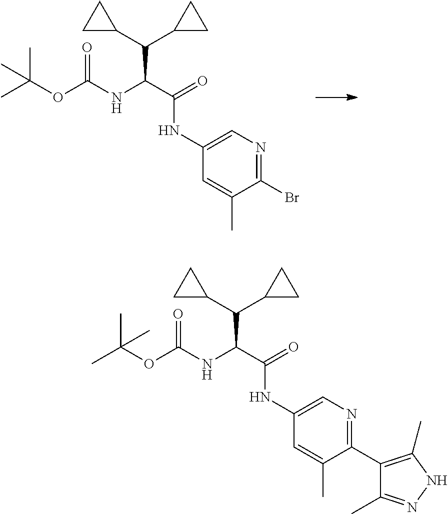 [SMILES: Cc1cc(NC(=O)[C@@H](NC(=O)OC(C)(C)C)C(C2CC2)C2CC2)cnc1Br ; Cc1cc(NC(=O)[C@@H](NC(=O)OC(C)(C)C)C(C2CC2)C2CC2)cnc1-c1c(C)n[nH]c1C]

According to the method of Preparation 7 the compound of Preparation 59 (41 mg, 0.095 mmol) was reacted with 3,5-dimethyl-4-(4,4,5,5-tetramethyl-1,3,2-dioxaborolan-2-yl)-1H-pyrazole (62.3 mg, 0.28 mmol). The crude filtrate was purified by acidic prep. HPLC to afford the title compound (24 mg, 57% yield). LCMS (METHOD 3) (ES): m/z 454.5 [M+H]⁺, RT=0.72 min.

### Preparation 66: tert-butyl N-[(1S)-1-(dicyclopropylmethyl)-2-[[6-(3,5-dimethyl-1H-pyrazol-4-yl)-5-methoxy-3-pyridyl]amino]-2-oxo-ethyl]carbamate

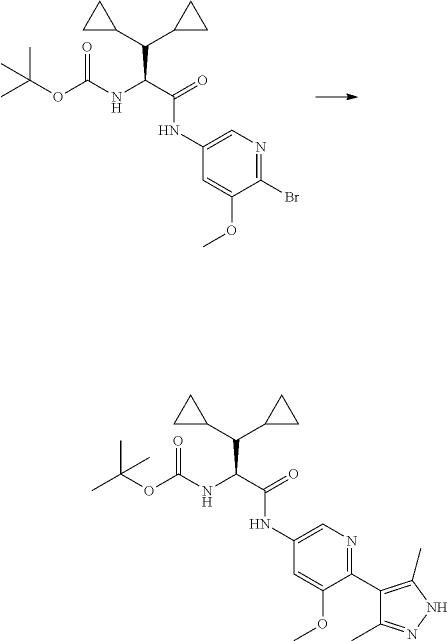 [SMILES: COc1cc(NC(=O)[C@@H](NC(=O)OC(C)(C)C)C(C2CC2)C2CC2)cnc1Br ; COc1cc(NC(=O)[C@@H](NC(=O)OC(C)(C)C)C(C2CC2)C2CC2)cnc1-c1c(C)n[nH]c1C]

According to the method of Preparation 7 the compound of Preparation 60 (37 mg, 0.081 mmol) was reacted with 3,5-dimethyl-4-(4,4,5,5-tetramethyl-1,3,2-dioxaborolan-2-yl)-1H-pyrazole (54.2 mg, 0.244 mmol). The crude filtrate was purified by acidic prep. HPLC to afford the title compound (40 mg, 100% yield). LCMS (METHOD 3) (ES): m/z 470.3 [M+H]⁺, RT=0.71 min.

### Preparation 67: (2S)-2-amino-3,3-dicyclopropyl-N-[5-(3,5-dimethyl-1H-pyrazol-4-yl)pyrazin-2-yl]propenamide

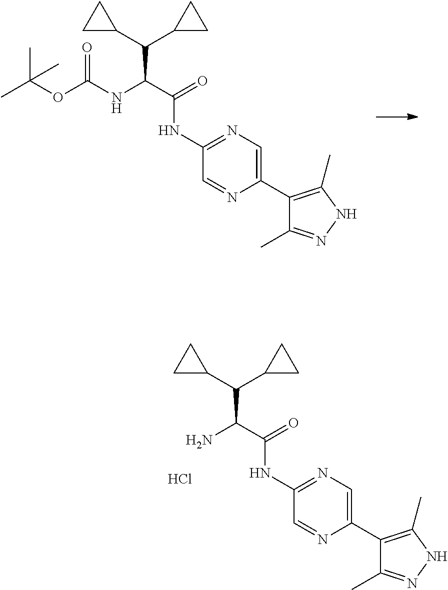 [SMILES: Cc1n[nH]c(C)c1-c1cnc(NC(=O)[C@@H](NC(=O)OC(C)(C)C)C(C2CC2)C2CC2)cn1 ; Cl ; Cc1n[nH]c(C)c1-c1cnc(NC(=O)[C@@H](N)C(C2CC2)C2CC2)cn1]

Hydrogen chloride (4M in 1,4-dioxane, 2 mL) was added to a solution of the product from Preparation 61 (38 mg, 0.086 mmol) in MeOH (1 mL) and the reaction mixture was stirred at room temperature for 1 hour then concentrated in vacuo to leave the title compound as an off-white solid (32 mg, assume 100% yield). Used without purification. LCMS (METHOD 3) (ES): m/z 341.2 [M+H]⁺, RT=0.47 min.

### Preparation 68: (2S)-2-amino-3,3-dicyclopropyl-N-[5-(3,5-dimethyl-1H-pyrazol-4-yl)pyrimidin-2-yl]propenamide hydrochloride

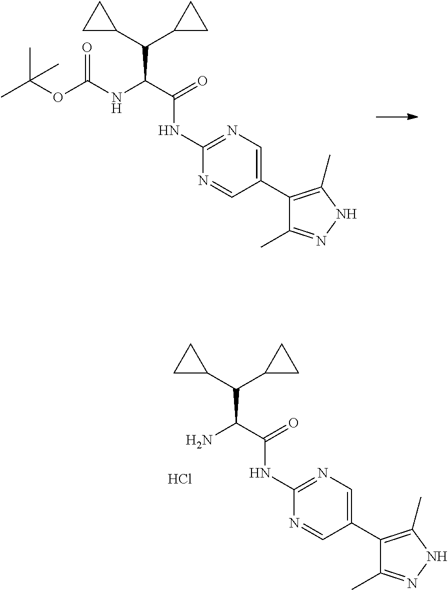 [SMILES: Cc1n[nH]c(C)c1-c1cnc(NC(=O)[C@@H](NC(=O)OC(C)(C)C)C(C2CC2)C2CC2)nc1 ; Cl ; Cc1n[nH]c(C)c1-c1cnc(NC(=O)[C@@H](N)C(C2CC2)C2CC2)nc1]

Hydrogen chloride (4M in 1,4-dioxane, 2 mL) was added to a solution of the product from Preparation 62 (53 mg, 0.12 mmol) in MeOH (1 mL) and the reaction mixture was stirred at room temperature for 1 hour then concentrated in vacuo to leave the title compound as an off-white solid (45 mg, assume 100% yield). Used without purification. LCMS (METHOD 3) (ES): m/z 341.2 [M+H]⁺, RT=0.43 min.

### Preparation 69: (2S)-2-amino-3,3-dicyclopropyl-N-[6-(3,5-dimethyl-1H-pyrazol-4-yl)pyridazin-3-yl]propenamide hydrochloride

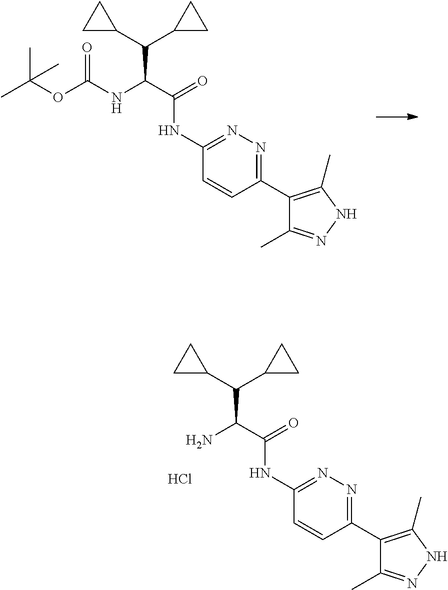 [SMILES: Cc1n[nH]c(C)c1-c1ccc(NC(=O)[C@@H](NC(=O)OC(C)(C)C)C(C2CC2)C2CC2)nn1 ; Cl ; Cc1n[nH]c(C)c1-c1ccc(NC(=O)[C@@H](N)C(C2CC2)C2CC2)nn1]

Hydrogen chloride (4M in 1,4-dioxane, 2 mL) was added to a solution of the product from Preparation 63 (27 mg, 0.061 mmol) in MeOH (1 mL) and the reaction mixture was stirred at room temperature for 1 hour then concentrated in vacuo to leave the title compound as an off-white solid (23 mg, assume 100% yield). Used without purification. LCMS (METHOD 3) (ES): m/z 341.2 [M+H]⁺, RT=0.45 min.

### Preparation 70: (2S)-2-amino-3,3-dicyclopropyl-N-[2-(3,5-dimethyl-1H-pyrazol-4-yl)pyrimidin-5-yl]propenamide hydrochloride

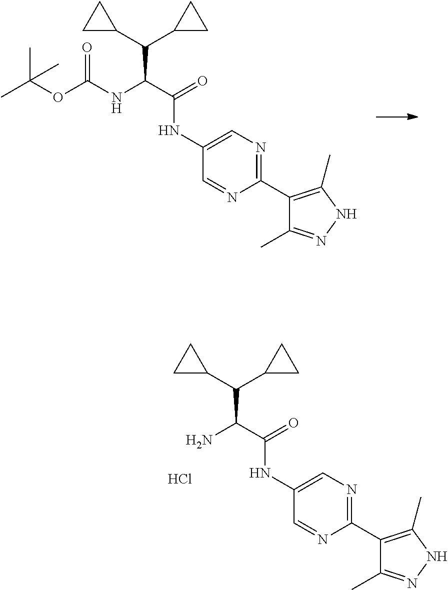 [SMILES: Cc1n[nH]c(C)c1-c1ncc(NC(=O)[C@@H](NC(=O)OC(C)(C)C)C(C2CC2)C2CC2)cn1 ; Cl ; Cc1n[nH]c(C)c1-c1ncc(NC(=O)[C@@H](N)C(C2CC2)C2CC2)cn1]

Hydrogen chloride (4M in 1,4-dioxane, 2 mL) was added to a solution of the product from Preparation 64 (10 mg, 0.023 mmol) in MeOH (1 mL) and the reaction mixture was stirred at room temperature for 1 hour then concentrated in vacuo to leave the title compound as an off-white solid (9 mg, assume 100% yield). Used without purification. LCMS (METHOD 3) (ES): m/z 341.2 [M+H]⁺, RT=0.50 min.

### Preparation 71: (2S)-2-amino-3,3-dicyclopropyl-N-[6-(3,5-dimethyl-1H-pyrazol-4-yl)-5-methyl-3-pyridyl]propenamide hydrochloride

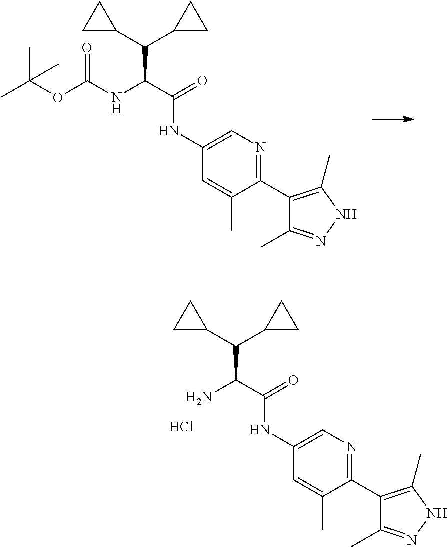 [SMILES: Cc1cc(NC(=O)[C@@H](NC(=O)OC(C)(C)C)C(C2CC2)C2CC2)cnc1-c1c(C)n[nH]c1C ; Cc1cc(NC(=O)[C@@H](N)C(C2CC2)C2CC2)cnc1-c1c(C)n[nH]c1C ; Cl]

Hydrogen chloride (4M in 1,4-dioxane, 2 mL) was added to a solution of the product from Preparation 65 (23 mg, 0.05 mmol) in MeOH (1 mL) and the reaction mixture was stirred at room temperature for 1 hour then concentrated in vacuo to leave the title compound as an off-white solid (21 mg, assume 100% yield). Used without purification. LCMS (METHOD 3) (ES): m/z 354.2 [M+H]⁺, RT=0.49 min.

### Preparation 72: (2S)-2-amino-3,3-dicyclopropyl-N-[6-(3,5-dimethyl-1H-pyrazol-4-yl)-5-methoxy-3-pyridyl]propenamide hydrochloride

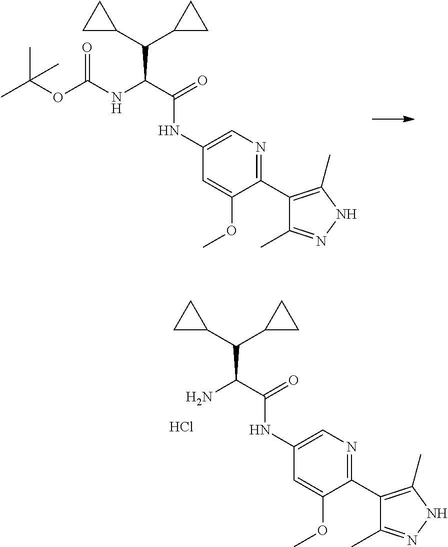 [SMILES: COc1cc(NC(=O)[C@@H](NC(=O)OC(C)(C)C)C(C2CC2)C2CC2)cnc1-c1c(C)n[nH]c1C ; COc1cc(NC(=O)[C@@H](N)C(C2CC2)C2CC2)cnc1-c1c(C)n[nH]c1C ; Cl]

Hydrogen chloride (4M in 1,4-dioxane, 2 mL) was added to a solution of the product from Preparation 66 (48 mg, 0.102 mmol) in MeOH (1 mL) and the reaction mixture was stirred at room temperature for 1 hour then concentrated in vacuo to leave the title compound as an off-white solid (44 mg, assume 100% yield). Used without purification. LCMS (METHOD 3) (ES): m/z 370.2 [M+H]⁺, RT=0.48 min.

### Preparation 73: tert-butyl N-[(1S)-1-[(6-bromo-3-pyridyl)carbamoyl]-2,2-dicyclopropyl-ethyl]carbamate

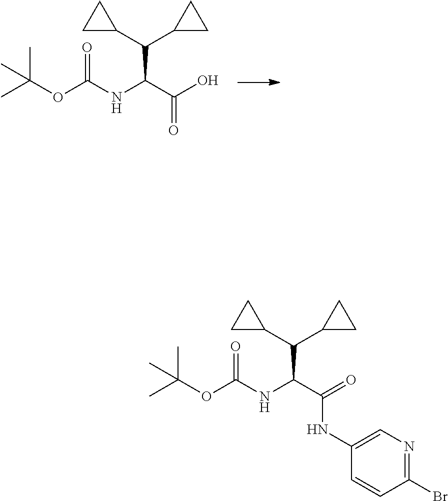 [SMILES: CC(C)(C)OC(=O)N[C@H](C(=O)O)C(C1CC1)C1CC1 ; CC(C)(C)OC(=O)N[C@H](C(=O)Nc1ccc(Br)nc1)C(C1CC1)C1CC1]

According to the method of Preparation 58 the product of Preparation 36 (200 mg, 0.743 mmol) was reacted with 6-bromopyridin-3-amine (141 mg, 0.817 mmol) to afford the title compound after prep. basic HPLC (120 mg, 38% yield). 1H NMR (400 MHz, CDCl₃) δ 8.48 (s, 1H), 8.36 (d, J=2.8 Hz, 1H), 8.01 (dd, J=8.7, 2.9 Hz, 1H), 7.41 (d, J=8.6 Hz, 1H), 5.40 (s, 1H), 4.38 (dd, J=8.0, 4.9 Hz, 1H), 1.47 (s, 9H), 1.04-0.11 (m, 11H); LCMS (METHOD 3) (ES): m/z 424.3 [M−H]⁻, RT=0.83 min.

### Preparation 74: tert-butyl N-[(1S)-1-(dicyclopropylmethyl)-2-[[6-[5-ethyl-3-methyl-1-(2-trimethylsilylethoxymethyl)pyrazol-4-yl]-3-pyridyl]amino]-2-oxo-ethyl]carbamate

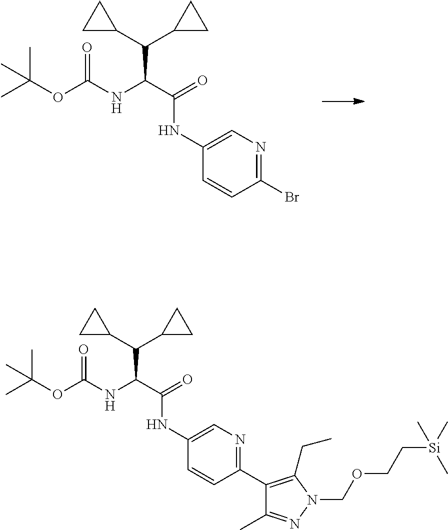 [SMILES: CC(C)(C)OC(=O)N[C@H](C(=O)Nc1ccc(Br)nc1)C(C1CC1)C1CC1 ; CCc1c(-c2ccc(NC(=O)[C@@H](NC(=O)OC(C)(C)C)C(C3CC3)C3CC3)cn2)c(C)nn1COCC[Si](C)(C)C]

According to the method of Preparation 7 the product of Preparation 73 (120 mg, 0.283 mmol) was reacted with the product of Preparation 6 (103 mg, 0.283 mmol) to afford the title compound after prep. acidic HPLC (113 mg, 68% yield). 1H NMR (400 MHz, MeOD) δ 8.84-8.69 (m, 1H), 8.24-8.10 (m, 1H), 7.42 (dd, J=8.6, 6.6 Hz, 1H), 5.48-5.32 (m, 2H), 4.48-4.26 (m, 1H), 3.70-3.53 (m, 2H), 2.98-2.64 (m, 2H), 2.47-2.19 (m, 3H), 1.48 (s, 9H), 1.18-0.14 (m, 17H), −0.00-−0.02 (m, 9H); LCMS (METHOD 3) (ES): m/z 585.5 [M+H]⁺, RT=1.01 min.

### Preparation 75: (2S)-2-amino-3,3-dicyclopropyl-N-[6-[5-ethyl-3-methyl-1-(2-trimethylsilylethoxymethyl)pyrazol-4-yl]-3-pyridyl]propenamide hydrochloride

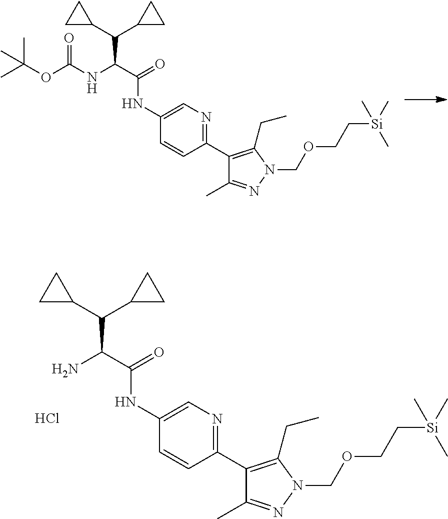 [SMILES: CCc1c(-c2ccc(NC(=O)[C@@H](NC(=O)OC(C)(C)C)C(C3CC3)C3CC3)cn2)c(C)nn1COCC[Si](C)(C)C ; CCc1c(-c2ccc(NC(=O)[C@@H](N)C(C3CC3)C3CC3)cn2)c(C)nn1COCC[Si](C)(C)C ; Cl]

According to the method of Preparation 67 the product of Preparation 74 (111 mg, 0.190 mmol) was reacted to afford the title compound (93 mg, assume 100% yield). LCMS (METHOD 3) (ES): m/z 484.3 [M+H]⁺, RT=0.69 min.

### Preparation 76: N-[(1S)-1-(dicyclopropylmethyl)-2-[[6-[5-ethyl-3-methyl-1-(2-trimethylsilylethoxymethyl)pyrazol-4-yl]-3-pyridyl]amino]-2-oxo-ethyl]-2-ethyl-pyrazole-3-carboxamide

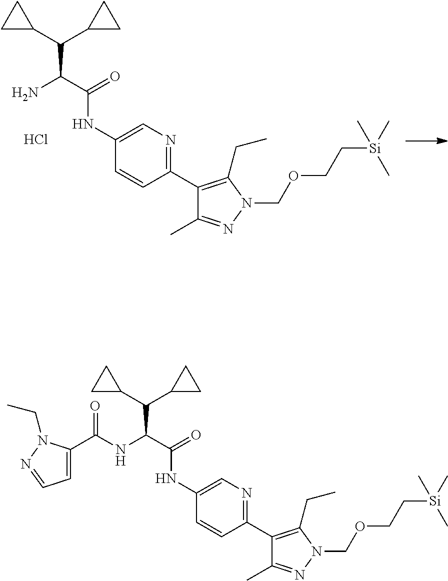

According to the method of Preparation 11 the product of Preparation 75 (23 mg, 0.048 mmol) was reacted with 2-ethylpyrazole-3-carboxylic acid (6.7 mg, 0.048 mmol) to afford the title compound after prep. acidic HPLC (16 mg, 55% yield). LCMS (METHOD 3) (ES): m/z 606.4 [M+H]⁺, RT=0.95 min.

### Preparation 77: N-[(1S)-1-(dicyclopropylmethyl)-2-[[6-[5-ethyl-3-methyl-1-(2-trimethylsilylethoxymethyl)pyrazol-4-yl]-3-pyridyl]amino]-2-oxo-ethyl]-2-(3-methoxypropyl)pyrazole-3-carboxamide

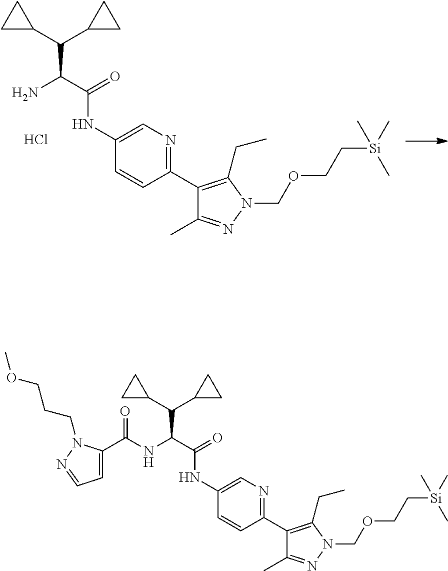

According to the method of Preparation 11 the product of Preparation 75 (23 mg, 0.048 mmol) was reacted with the product from Preparation 10 (8.8 mg, 0.048 mmol) to afford the title compound after prep. acidic HPLC (17 mg, 55% yield). LCMS (METHOD 3) (ES): m/z 650.4 [M+H]⁺, RT=0.95 min.

### Preparation 78: N-[(1S)-1-(dicyclopropylmethyl)-2-[[6-[5-ethyl-3-methyl-1-(2-trimethylsilylethoxymethyl)pyrazol-4-yl]-3-pyridyl]amino]-2-oxo-ethyl]-2-(2-methoxyethyl)pyrazole-3-carboxamide

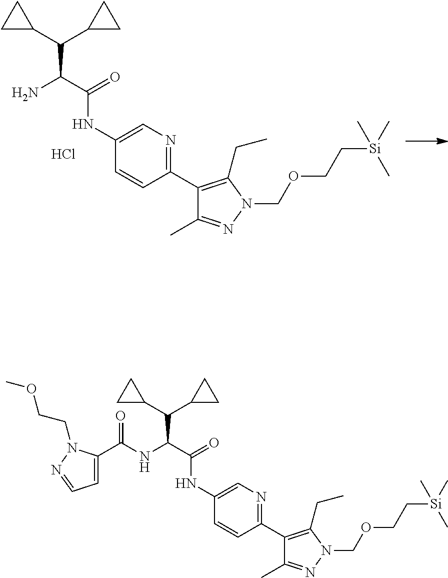

According to the method of Preparation 11 the product of Preparation 75 (23 mg, 0.048 mmol) was reacted with 2-(2-methoxyethyl)pyrazole-3-carboxylic acid (8.8 mg, 0.048 mmol) to afford the title compound after prep. acidic HPLC (16 mg, 53% yield). LCMS (METHOD 3) (ES): m/z 636.4 [M+H]⁺, RT=0.93 min.

### Preparation 79: N-[(1S)-1-(dicyclopropylmethyl)-2-[[6-[5-ethyl-3-methyl-1-(2-trimethylsilylethoxymethyl)pyrazol-4-yl]-3-pyridyl]amino]-2-oxo-ethyl]-2-isopropylpyrazole-3-carboxamide

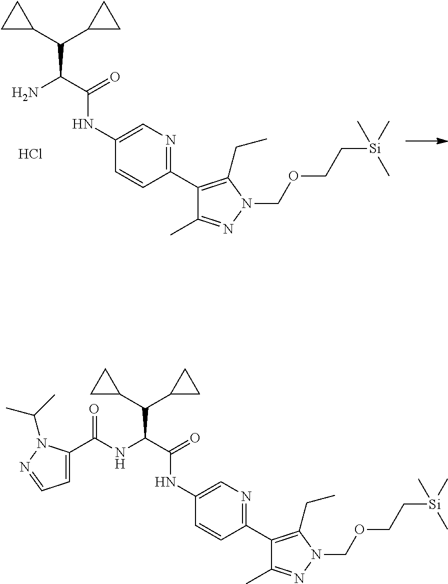

According to the method of Preparation 11 the product of Preparation 75 (23 mg, 0.048 mmol) was reacted with 2-isopropylpyrazole-3-carboxylic acid (7.4 mg, 0.048 mmol) to afford the title compound after prep. acidic HPLC (10 mg, 34% yield). LCMS (METHOD 3) (ES): m/z 620.4 [M+H]⁺, RT=0.98 min.

### Preparation 80: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-3-methyl-isoxazole-4-carboxamide

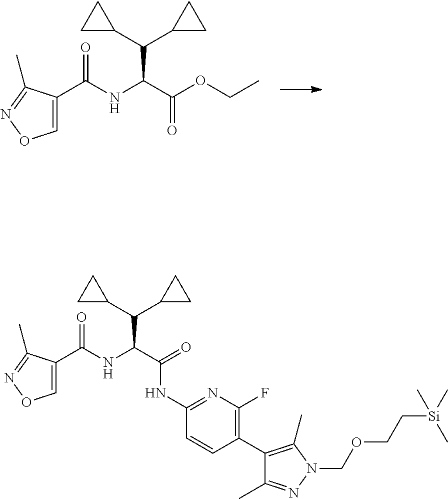 [SMILES: CCOC(=O)[C@@H](NC(=O)c1conc1C)C(C1CC1)C1CC1 ; Cc1nocc1C(=O)N[C@H](C(=O)Nc1ccc(-c2c(C)nn(COCC[Si](C)(C)C)c2C)c(F)n1)C(C1CC1)C1CC1]

According to the method of Preparation 27 the product of Preparation 49 (50 mg, 0.163 mmol) was reacted with the product from Preparation 41 (57.7 mg, 0.171 mmol) to afford the title compound after prep. acidic HPLC (45 mg, 46% yield). LCMS (METHOD 3) (ES): m/z 597.3 [M+H]⁺, RT=0.95 min.

### Preparation 81: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-3-ethyl-isoxazole-4-carboxamide

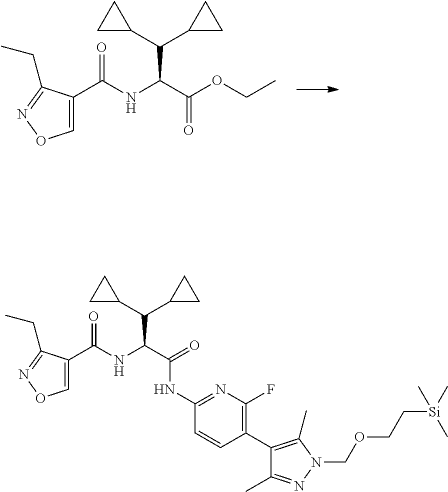

According to the method of Preparation 27 the product of Preparation 50 (49 mg, 0.153 mmol) was reacted with the product from Preparation 41 (54 mg, 0.161 mmol) to afford the title compound after prep. acidic HPLC (36 mg, 38% yield). LCMS (METHOD 3) (ES): m/z 611.3 [M+H]⁺, RT=0.98 min.

### Preparation 82: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-3-isopropyl-isoxazole-4-carboxamide

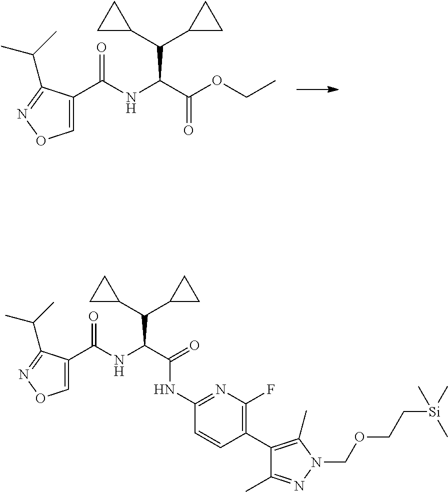

According to the method of Preparation 27 the product of Preparation 51 (36 mg, 0.108 mmol) was reacted with the product from Preparation 41 (38 mg, 0.113 mmol) to afford the title compound after prep. acidic HPLC (33 mg, 49% yield). LCMS (METHOD 3) (ES): m/z 625.3 [M+H]⁺, RT=1.00 min.

### Preparation 83: ethyl 2-but-3-enylpyrazole-3-carboxylate

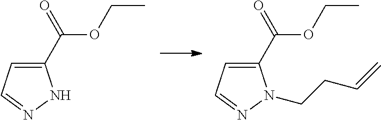

According to the method of Preparation 9, ethyl 1H-pyrazole-5-carboxylate (6.0 g, 43.0 mmol) was reacted with but-3-ene-1-ol (4.40 mg, 51.0 mmol). The obtained crude compound was purified by silica column chromatography (230-400 mesh), eluting with EtOAc in heptane, to afford the title compound as a colourless oil (7.1 g, 85% yield). 1H NMR (600 MHz, CDCl₃) δ 7.47 (d, J=2.0 Hz, 1H), 6.82 (d, J=2.0 Hz, 1H), 5.79 (ddt, J=17.2, 10.2, 6.9 Hz, 1H), 5.10-4.95 (m, 2H), 4.75-4.52 (m, 2H), 4.35 (q, J=7.1 Hz, 2H), 2.68-2.52 (m, 2H), 1.38 (t, J=7.1 Hz, 3H); LCMS (METHOD 3) (ES): m/z 195.3 [M+H]⁺, RT=0.72 min.

### Preparation 84: ethyl 2-(3-oxopropyl)pyrazole-3-carboxylate

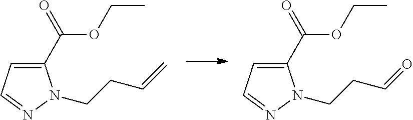

Osmium tetroxide (2.5% solution in tert-butanol, 0.65 mL, 0.052 mmol) was added to a solution of the product from Preparation 83 (1.0 g, 5.15 mmol) in THF:water (25 mL:20 mL) at room temperature. NaIO₄ (2.75 g, 12.9 mmol) was added portion-wise over 10 minutes to the now dark solution. The reaction mixture was stirred for 18 hours, then filtered. The filtrate was extracted with Et₂O (2×40 mL). The organic layer was washed with Na₂S₂O₃ (1% solution, 10 mL), dried over Na₂SO₄, filtered and concentrated in vacuo. The obtained crude compound was purified by silica column chromatography (230-400 mesh), eluting with EtOAc in heptane, to afford the title compound as an orange oil (667 mg, 66% yield). 1H NMR (300 MHz, CDCl₃) δ 9.84 (t, J=1.3 Hz, 1H), 7.48 (d, J=2.0 Hz, 1H), 6.83 (d, J=2.0 Hz, 1H), 4.92 (t, J=6.8 Hz, 2H), 4.35 (q, J=7.1 Hz, 2H), 3.03 (td, J=6.8, 1.3 Hz, 2H), 1.38 (t, J=7.1 Hz, 3H).

### Preparation 85: ethyl 2-(4,4,4-trifluoro-3-trimethylsilyloxy-butyl)pyrazole-3-carboxylate

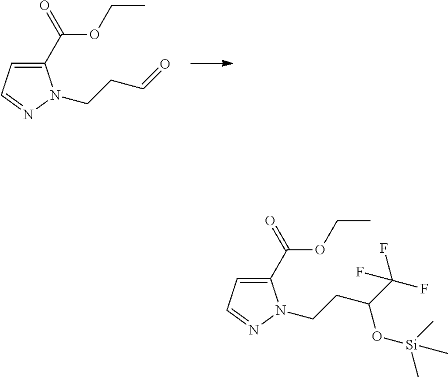 [SMILES: CCOC(=O)c1ccnn1CCC=O ; CCOC(=O)c1ccnn1CCC(O[Si](C)(C)C)C(F)(F)F]

Trimethyl(trifluoromethyl)silane (3.31 mL, 22.4 mmol) was added, dropwise over 5 minutes, to a solution of the product from Preparation 84 (4.00 g, 20.4 mmol) and CsF (31.0 mg, 0.204 mmol) in anhydrous THF (41 mL) at 5° C. The reaction mixture was stirred at room temperature over 2 hours. The reaction mixture was quenched with water and extracted with EtOAc (2×100 mL). The combined organic phase was washed with brine solution, dried over Na₂SO₄, filtered and concentrated in vacuo. The obtained crude compound was purified by silica column chromatography (230-400 mesh), eluting with EtOAc in heptane, to afford the title compound (4.42 g, 81% yield). LCMS (METHOD 3) (ES): m/z 339.3 [M+H]⁺, RT=0.95 min.

### Preparation 86: ethyl 2-(4,4,4-trifluoro-3-hydroxy-butyl)pyrazole-3-carboxylate

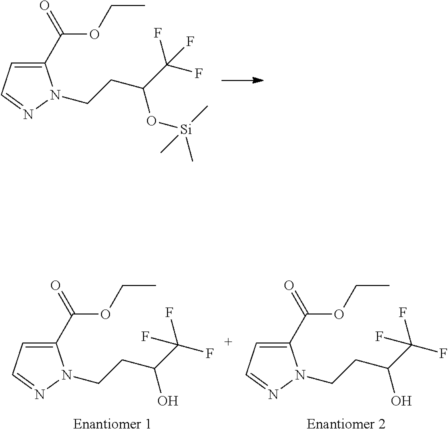

Citric acid (aq. solution, 45 mL, 24 mmol) was added to a solution of the product from Preparation 85 (4.00 g, 12 mmol) in MeOH (60 mL) and stirred at room temperature for 2 hours. The reaction mixture was partitioned between aqueous brine and EtOAc. The organic layer was collected, dried over Na₂SO₄, filtered and concentrated in vacuo to afford racemic compound as a colourless gum (3.02 g, 96% yield). The two enantiomers were separated by preparative chiral HPLC (Column: Lux C₃ (21.2 mm×250 mm, 5 μm), Eluent: 90:10 Heptane: IPA, Flow rate: 21 mL/min) giving Preparation 86a (Enantiomer 1, 1.239 g, RT=6.39 min) and Preparation 86b (Enantiomer 2, 1.277 g, RT=7.32 min.)

### Preparation 87: Enantiomer 1 of 2-(4,4,4-trifluoro-3-hydroxy-butyl)pyrazole-3-carboxylic acid

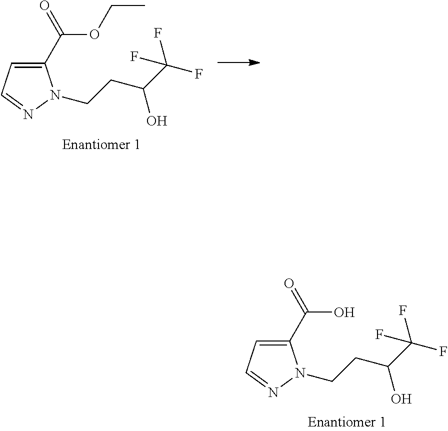 [SMILES: CCOC(=O)c1ccnn1CCC(O)C(F)(F)F ; O=C(O)c1ccnn1CCC(O)C(F)(F)F]

LiOH (54 mg, 2.25 mmol) was added to a solution of the product from Preparation 86a (200 mg, 0.75 mmol) in MeOH:water (3.75 mL:1.85 mL) at room temperature and stirred for 1 hour. The reaction mixture was concentrated to low volume. Citric acid (10% aq. solution) was added to adjust to pH 3-4. The reaction mixture was extracted with EtOAc (2×10 mL). The combined organic phase was dried over Na₂SO₄, filtered and concentrated in vacuo to afford the title compound (166 mg, 92% yield). LCMS (METHOD 3) (ES): m/z 239.1 [M+H]⁺, RT=0.41 min.

### Preparation 88: Enantiomer 2 of 2-(4,4,4-trifluoro-3-hydroxy-butyl)pyrazole-3-carboxylic acid

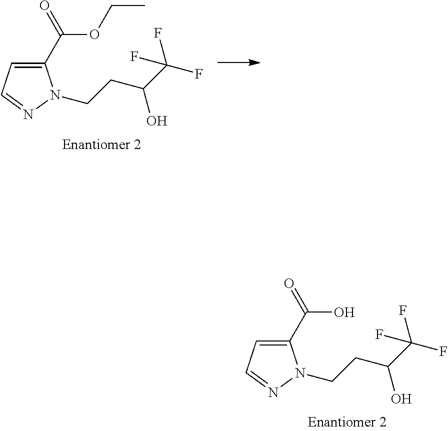 [SMILES: CCOC(=O)c1ccnn1CCC(O)C(F)(F)F ; O=C(O)c1ccnn1CCC(O)C(F)(F)F]

LiOH (54 mg, 2.25 mmol) was added to a solution of the product from Preparation 86b (200 mg, 0.75 mmol) in MeOH:water (3.75 mL:1.85 mL) at room temperature and stirred for 1 hour. The reaction mixture was concentrated to low volume. Citric acid (10% aq. solution) was added to adjust to pH 3-4. The reaction mixture was extracted with EtOAc (2×10 mL). The combined organic phase was dried over Na₂SO₄, filtered and concentrated in vacuo to afford the title compound (175 mg, 97% yield). LCMS (METHOD 3) (ES): m/z 239.1 [M+H]⁺, RT=0.41 min.

### Preparation 89: tert-butyl N-[(1S)-1-carbamoyl-2,2-dicyclopropyl-ethyl]carbamate

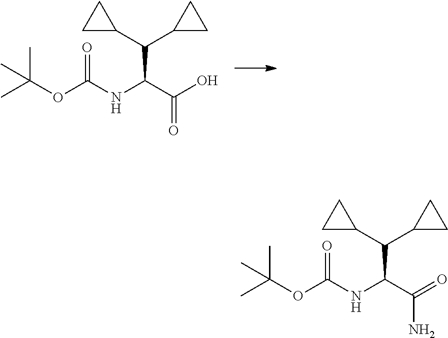 [SMILES: CC(C)(C)OC(=O)N[C@H](C(=O)O)C(C1CC1)C1CC1 ; CC(C)(C)OC(=O)N[C@H](C(N)=O)C(C1CC1)C1CC1]

Ammonium bicarbonate (6.11 g, 77.2 mmol) was added to a solution of the product from Preparation 36 (16.0 g, 59.4 mmol), tert-butoxycarbonyl tert-butyl carbonate (16.9 g, 77.2 mmol) and pyridine (2.40 mL, 29.7 mmol) in 1,4-dioxane (150 mL) and the reaction mixture was stirred at room temperature for 18 hours. The reaction mixture was concentrated to low volume then diluted with water (200 mL). After stirring for 10 minutes the product was collected by filtration and dried in vacuo to leave a colourless solid (14.26 g, 89% yield). 1H NMR (400 MHz, DMSO-d6) δ 7.21 (s, 1H), 6.97 (s, 1H), 6.45 (d, J=9.4 Hz, 1H), 4.08 (dd, J=9.5, 5.0 Hz, 1H), 1.39 (s, 9H), 0.83-0.61 (m, 2H), 0.47 (ddd, J=24.7, 8.9, 4.6 Hz, 2H), 0.30 (dtt, J=21.6, 8.5, 4.1 Hz, 3H), 0.24-0.06 (m, 4H).

### Preparation 90: tert-butyl N-[(1S)-1-[(5-bromo-6-fluoro-2-pyridyl)carbamoyl]-2,2-dicyclopropyl-ethyl]carbamate

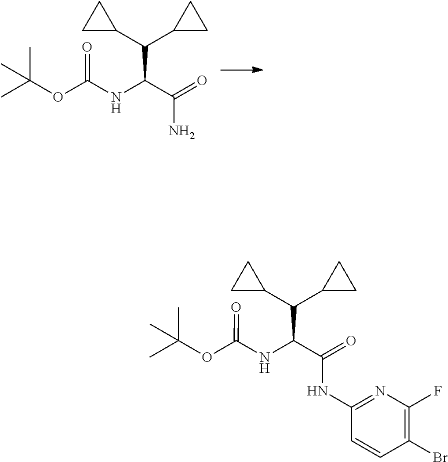 [SMILES: CC(C)(C)OC(=O)N[C@H](C(N)=O)C(C1CC1)C1CC1 ; CC(C)(C)OC(=O)N[C@H](C(=O)Nc1ccc(Br)c(F)n1)C(C1CC1)C1CC1]

K₂CO₃ (1.03 g, 7.45 mmol) was added to a solution of the product from Preparation 89 (1.00 g, 3.73 mmol) and 3,6-dibromo-2-fluoro-pyridine (1.165 g, 4.57 mmol) in anhydrous THF (10 mL). The solution was degassed for 10 minutes with argon. Palladium (II) acetate (16.7 mg, 0.0745 mmol) and Xantphos (86.2 mg, 0.149 mmol) were added, the reaction was sealed and stirred at 75° C. for 18 hours. The reaction mixture was concentrated in vacuo, then partitioned between water (20 mL) and EtOAc (40 mL). The organic phase was dried over Na₂SO₄, filtered and concentrated in vacuo. Trituration with Et₂O afforded the title compound as a colourless solid (1.22 g, 74% yield). LCMS (METHOD 3) (ES): m/z 440.2 [M+H]⁺, RT=0.94 min.

### Preparation 91: tert-butyl N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]carbamate

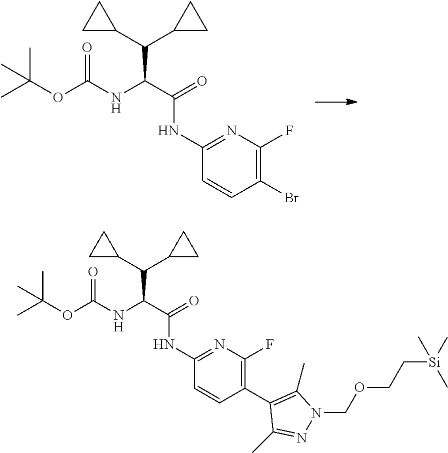 [SMILES: CC(C)(C)OC(=O)N[C@H](C(=O)Nc1ccc(Br)c(F)n1)C(C1CC1)C1CC1 ; Cc1nn(COCC[Si](C)(C)C)c(C)c1-c1ccc(NC(=O)[C@@H](NC(=O)OC(C)(C)C)C(C2CC2)C2CC2)nc1F]

According to the method of Preparation 22 the product of Preparation 90 (1.22 g, 2.76 mmol) was reacted with the product from Preparation 21 (1.17 g, 3.31 mmol). The obtained crude compound was purified by silica column chromatography (230-400 mesh), eluting with EtOAc in heptane, to afford the title compound (1.37 g, 84% yield). LCMS (METHOD 3) (ES): m/z 588.3 [M+H]⁺, RT=1.04 min.

### Preparation 92: (2S)-2-amino-3,3-dicyclopropyl-N-[5-[3,5-dimethyl-1-(2-trimethylsilylethoxymethyl)pyrazol-4-yl]-6-fluoro-2-pyridyl]propenamide hydrochloride

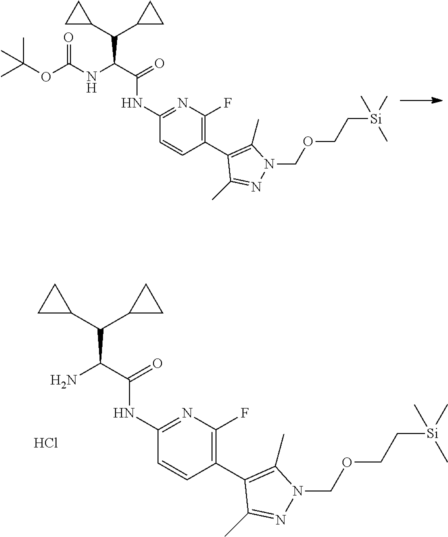 [SMILES: Cc1nn(COCC[Si](C)(C)C)c(C)c1-c1ccc(NC(=O)[C@@H](NC(=O)OC(C)(C)C)C(C2CC2)C2CC2)nc1F ; Cc1nn(COCC[Si](C)(C)C)c(C)c1-c1ccc(NC(=O)[C@@H](N)C(C2CC2)C2CC2)nc1F ; Cl]

Hydrogen chloride (3M in CPME, 3.08 mL) was added to a solution of the product from Preparation 89 (1.36 g, 2.31 mmol) in DCM (5 mL) and the reaction mixture was stirred at room temperature for 2 hours then concentrated in vacuo to leave the title compound as an off-white solid (1.21 g, assume 100% yield). Used without purification.

### Preparation 93: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(4,4,4-trifluoro-3-hydroxy-butyl)pyrazole-3-carboxamide (Diastereomer 1)

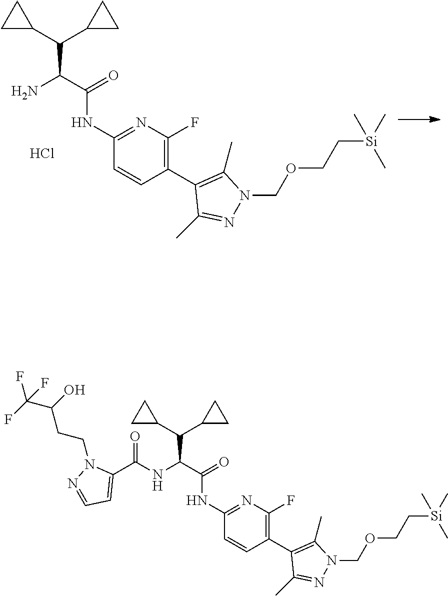 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2ccnn2CCC(O)C(F)(F)F)C(C2CC2)C2CC2)nc1F]

According to the method of Preparation 11 the product of Preparation 92 (20 mg, 0.038 mmol) was reacted with the product from Preparation 87 (10 mg, 0.042 mmol) to afford the title compound after prep. basic HPLC (11.7 mg, 43% yield). LCMS (METHOD 3) (ES): m/z 708.3 [M+H]⁺, RT=0.86 min.

### Preparation 94: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(4,4,4-trifluoro-3-hydroxy-butyl)pyrazole-3-carboxamide (Diastereomer 2)

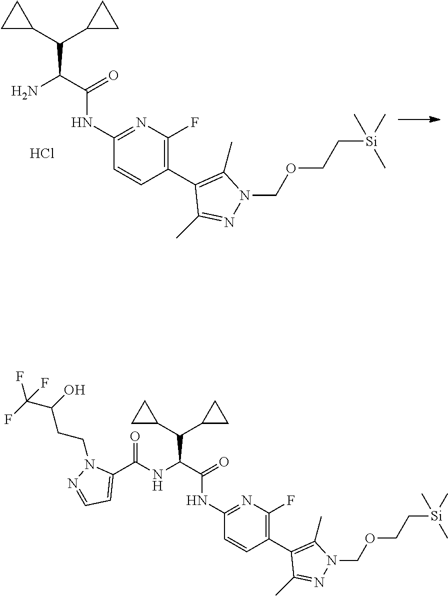 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2ccnn2CCC(O)C(F)(F)F)C(C2CC2)C2CC2)nc1F]

According to the method of Preparation 11 the product of Preparation 92 (20 mg, 0.038 mmol) was reacted with the product from Preparation 88 (10 mg, 0.042 mmol) to afford the title compound after prep. basic HPLC (12 mg, 44% yield). LCMS (METHOD 3) (ES): m/z 708.3 [M+H]⁺, RT=0.86 min.

### Preparation 95: ethyl (2S)-3,3-dicyclopropyl-2-[(3-ethyltriazole-4-carbonyl)amino]propanoate

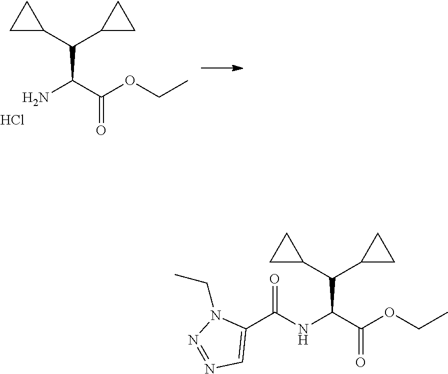 [SMILES: Cl ; CCOC(=O)[C@@H](N)C(C1CC1)C1CC1 ; CCOC(=O)[C@@H](NC(=O)c1cnnn1CC)C(C1CC1)C1CC1]

According to the method of Preparation 49 the product of Preparation 37 (150 mg, 0.642 mmol) was reacted with 3-ethyltriazole-4-carboxylic acid (99.6 mg, 0.706 mmol) to afford the title compound after prep. basic HPLC (124 mg, 60% yield). LCMS (METHOD 3) (ES): m/z 322.1 [M+H]⁺, RT=0.74 min.

### Preparation 96: ethyl (2S)-3,3-dicyclopropyl-2-[(3-isopropyltriazole-4-carbonyl)amino]propanoate

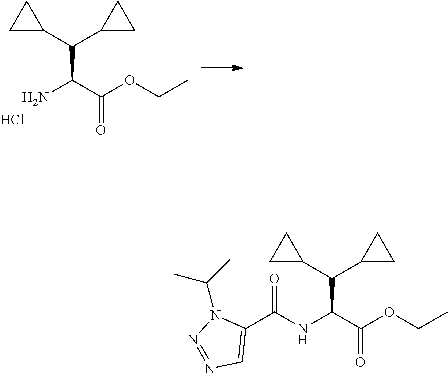 [SMILES: Cl ; CCOC(=O)[C@@H](N)C(C1CC1)C1CC1 ; CCOC(=O)[C@@H](NC(=O)c1cnnn1C(C)C)C(C1CC1)C1CC1]

According to the method of Preparation 49 the product of Preparation 37 (150 mg, 0.642 mmol) was reacted with 3-isopropyltriazole-4-carboxylic acid (110 mg, 0.706 mmol) to afford the title compound after prep. basic HPLC (160 mg, 74% yield). LCMS (METHOD 3) (ES): m/z 335.1 [M+H]⁺, RT=0.78 min.

### Preparation 97: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-3-ethyl-triazole-4-carboxamide

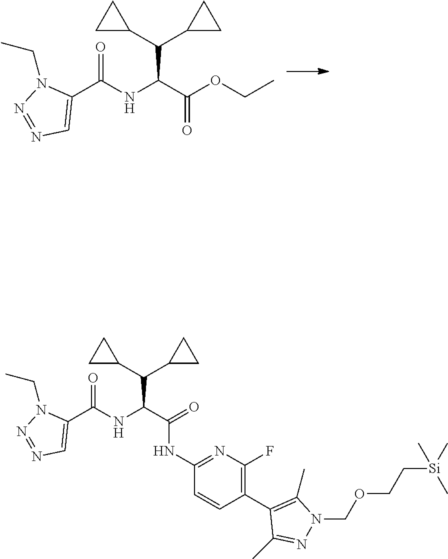

According to the method of Preparation 27 the product of Preparation 95 (50 mg, 0.156 mmol) was reacted with the product from Preparation 41 (55 mg, 0.164 mmol) to afford the title compound after prep. acidic HPLC (95 mg, assume 100% yield). LCMS (METHOD 3) (ES): m/z 611.3 [M+H]⁺, RT=0.94 min.

### Preparation 98: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-3-isopropyl-triazole-4-carboxamide

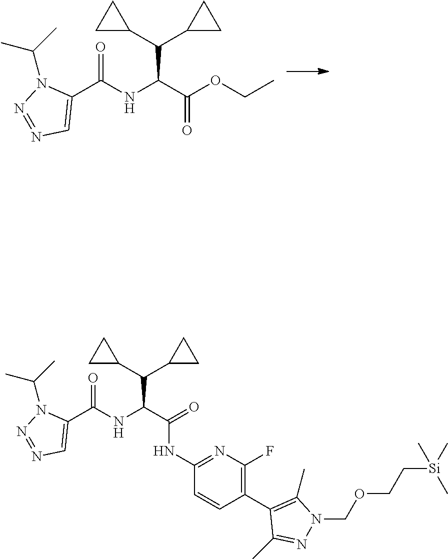

According to the method of Preparation 27 the product of Preparation 96 (50 mg, 0.15 mmol) was reacted with the product from Preparation 41 (53 mg, 0.157 mmol) to afford the title compound after prep. acidic HPLC (97 mg, assume 100% yield). LCMS (METHOD 3) (ES): m/z 625.4 [M+H]⁺, RT=0.97 min.

### Preparation 99: ethyl (2S)-3,3-dicyclopropyl-2-[(2-methylpyrazole-3-carbonyl)amino]propanoate

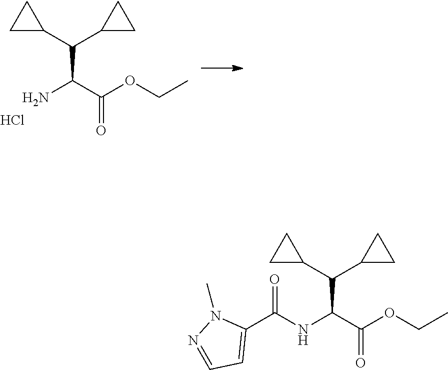 [SMILES: Cl ; CCOC(=O)[C@@H](N)C(C1CC1)C1CC1 ; CCOC(=O)[C@@H](NC(=O)c1ccnn1C)C(C1CC1)C1CC1]

According to the method of Preparation 49 the product of Preparation 37 (600 mg, 2.57 mmol) was reacted with 2-methylpyrazole-3-carboxylic acid (356 mg, 2.82 mmol). The obtained crude compound was purified by silica column chromatography (230-400 mesh), eluting with EtOAc in heptane, to afford the title compound (579 mg, 74% yield). LCMS (METHOD 3) (ES): m/z 306.1 [M+H]⁺, RT=0.74 min.

### Preparation 100: ethyl (2S)-3,3-dicyclopropyl-2-[(5-methyl-1-tetrahydropyran-4-yl-pyrazole-4-carbonyl)amino]propanoate

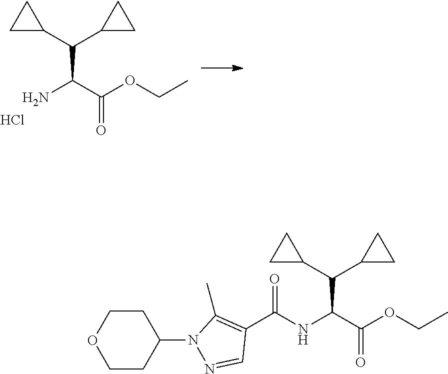 [SMILES: Cl ; CCOC(=O)[C@@H](N)C(C1CC1)C1CC1 ; CCOC(=O)[C@@H](NC(=O)c1cnn(C2CCOCC2)c1C)C(C1CC1)C1CC1]

According to the method of Preparation 49 the product of Preparation 37 (300 mg, 1.26 mmol) was reacted with 5-methyl-1-tetrahydropyran-4-yl-pyrazole-4-carboxylic acid (297 mg, 1.41 mmol). The obtained crude compound was purified by silica column chromatography (230-400 mesh), eluting with EtOAc in heptane, to afford the title compound (322 mg, 64% yield). LCMS (METHOD 3) (ES): m/z 390.2 [M+H]⁺, RT=0.73 min.

### Preparation 101: ethyl 2-(3-tetrahydropyran-2-yloxypropyl)pyrazole-3-carboxylate

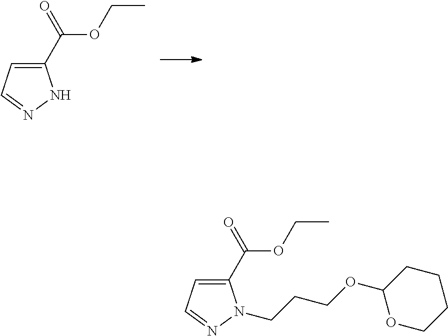

According to the method of Preparation 9, ethyl 1H-pyrazole-5-carboxylate (1.0 g, 7.14 mmol) was reacted with 3-tetrahydropyran-2-yloxypropan-1-ol (1.34 g, 8.39 mmol). The obtained crude compound was purified by silica column chromatography (230-400 mesh), eluting with EtOAc in heptane, to afford the title compound as a colourless oil (1.68 g, 83% yield). 1H NMR (400 MHz, CDCl₃) δ 7.47 (q, J=1.9 Hz, 1H), 6.83 (q, J=1.9 Hz, 1H), 4.78-4.61 (m, 2H), 4.57 (q, J=3.1 Hz, 1H), 4.34 (q, J=7.2 Hz, 2H), 3.91-3.70 (m, 2H), 3.55-3.31 (m, 2H), 2.14 (h, J=6.1, 5.6 Hz, 2H), 1.93-1.64 (m, 2H), 1.57-1.46, (m, 3H), 1.37 (td, J=7.2, 3.6 Hz, 3H), 1.26 (td, J=7.4, 6.9, 4.0 Hz, 1H).

### Preparation 102: 2-(3-tetrahydropyran-2-yloxypropyl)pyrazole-3-carboxylic acid

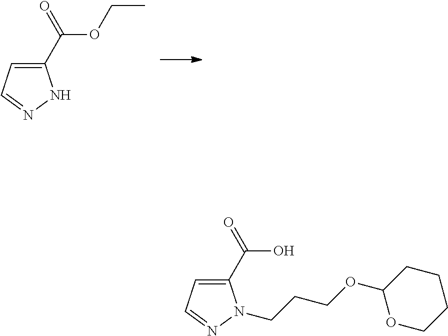

A solution of LiOH (499 mg, 11.9 mmol) in water (7.4 mL) was added to a solution of the product from Preparation 101 (1.68 g, 5.95 mmol) at room temperature and stirred for 1.5 hours. The reaction mixture was quenched with hydrogen chloride (4M in 1,4-dioxane) to pH 1. The reaction mixture was extracted with EtOAc (3×20 mL), dried over Na₂SO₄ and concentrated in vacuo to afford the title compound (1.65 g, 93% yield). LCMS (METHOD 3) (ES): m/z 253.2 [M−H]⁻, RT=0.51 min.

### Preparation 103: ethyl (2S)-3,3-dicyclopropyl-2-[[2-(3-tetrahydropyran-2-yloxypropyl)pyrazole-3-carbonyl]amino]propanoate

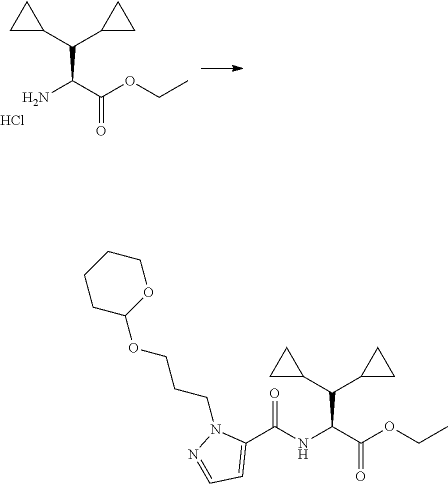 [SMILES: Cl ; CCOC(=O)[C@@H](N)C(C1CC1)C1CC1 ; CCOC(=O)[C@@H](NC(=O)c1ccnn1CCCOC1CCCCO1)C(C1CC1)C1CC1]

According to the method of Preparation 49 the product of Preparation 37 (300 mg, 1.26 mmol) was reacted with the product from Preparation 102 (359 mg, 1.41 mmol) to afford the title compound after prep. basic HPLC (273 mg, 49% yield).

### Preparation 104: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-methyl-pyrazole-3-carboxamide

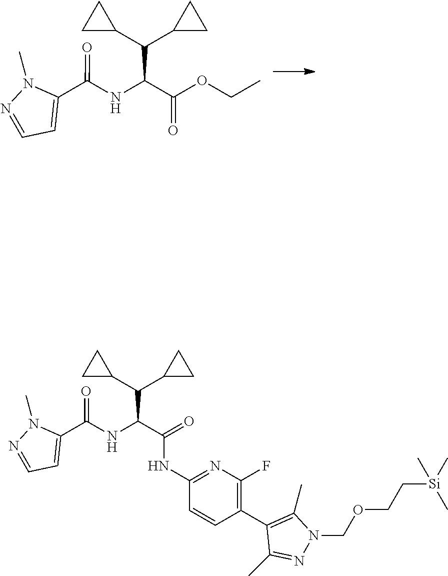 [SMILES: CCOC(=O)[C@@H](NC(=O)c1ccnn1C)C(C1CC1)C1CC1 ; Cc1nn(COCC[Si](C)(C)C)c(C)c1-c1ccc(NC(=O)[C@@H](NC(=O)c2ccnn2C)C(C2CC2)C2CC2)nc1F]

According to the method of Preparation 27 the product of Preparation 99 (60 mg, 0.196 mmol) was reacted with the product from Preparation 21 (69.4 mg, 0.206 mmol) to afford the title compound after prep. acidic HPLC (117 mg, assume 100% yield). LCMS (METHOD 3) (ES): m/z 596.3 [M+H]⁺, RT=0.95 min.

### Preparation 105: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(3-tetrahydropyran-2-yloxypropyl)pyrazole-3-carboxamide

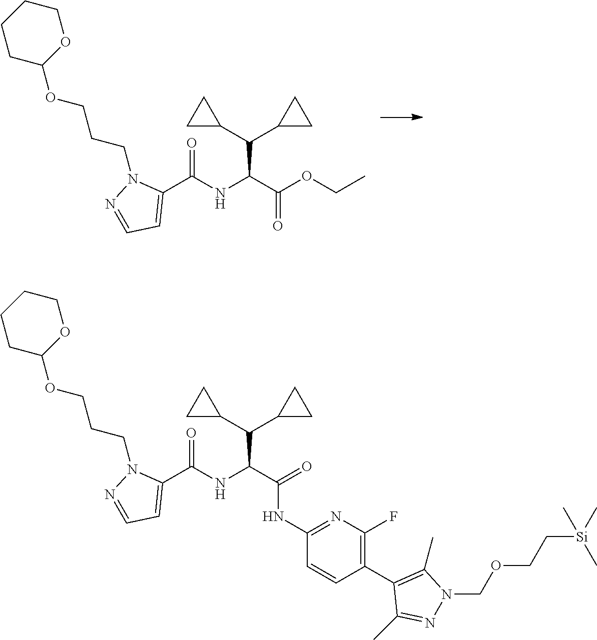 [SMILES: CCOC(=O)[C@@H](NC(=O)c1ccnn1CCCOC1CCCCO1)C(C1CC1)C1CC1 ; Cc1nn(COCC[Si](C)(C)C)c(C)c1-c1ccc(NC(=O)[C@@H](NC(=O)c2ccnn2CCCOC2CCCCO2)C(C2CC2)C2CC2)nc1F]

According to the method of Preparation 27 the product of Preparation 103 (60 mg, 0.138 mmol) was reacted with the product from Preparation 21 (49 mg, 0.145 mmol) to afford the title compound after prep. acidic HPLC (100 mg, assume 100% yield). LCMS (METHOD 3) (ES): m/z 724.3 [M+H]⁺, RT=1.03 min.

### Preparation 106: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-5-methyl-1-tetrahydropyran-4-yl-pyrazole-4-carboxamide

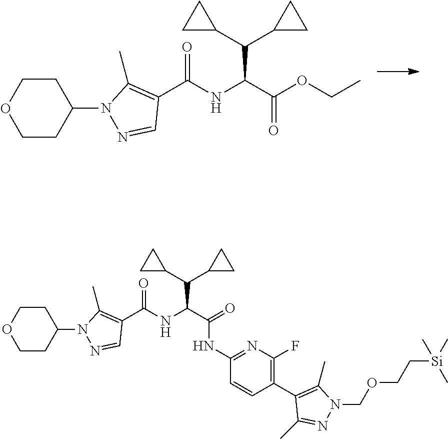 [SMILES: CCOC(=O)[C@@H](NC(=O)c1cnn(C2CCOCC2)c1C)C(C1CC1)C1CC1 ; Cc1nn(COCC[Si](C)(C)C)c(C)c1-c1ccc(NC(=O)[C@@H](NC(=O)c2cnn(C3CCOCC3)c2C)C(C2CC2)C2CC2)nc1F]

According to the method of Preparation 27 the product of Preparation 100 (60 mg, 0.154 mmol) was reacted with the product from Preparation 21 (54.4 mg, 0.162 mmol) to afford the title compound after prep. acidic HPLC (104 mg, assume 100% yield). LCMS (METHOD 3) (ES): m/z 680.4 [M+H]⁺, RT=0.94 min.

### Preparation 107: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-3-ethyl-triazole-4-carboxamide

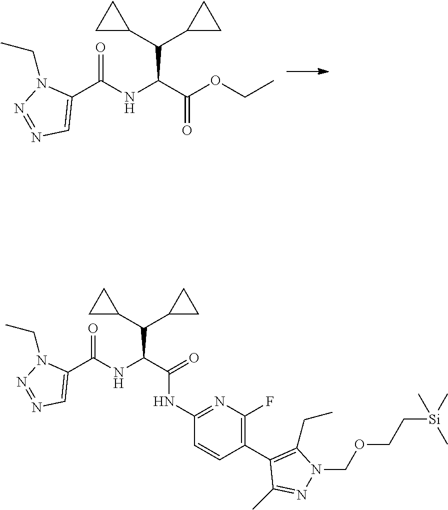

According to the method of Preparation 27 the product of Preparation 95 (50 mg, 0.156 mmol) was reacted with the product from Preparation 39 (57 mg, 0.164 mmol) to afford the title compound after prep. acidic HPLC (97 mg, assume 100% yield). LCMS (METHOD 3) (ES): m/z 625.4 [M+H]⁺, RT=0.96 min.

### Preparation 108: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-3-isopropyl-triazole-4-carboxamide

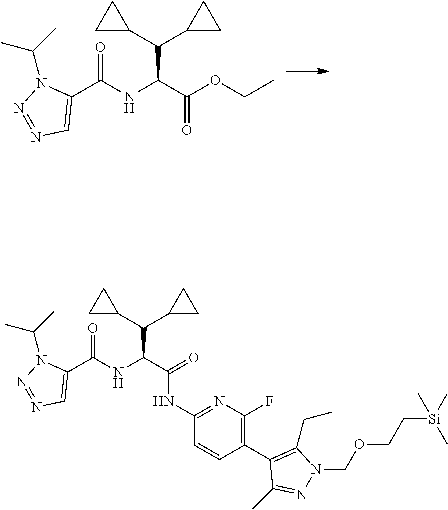

According to the method of Preparation 27 the product of Preparation 96 (50 mg, 0.15 mmol) was reacted with the product from Preparation 39 (55 mg, 0.157 mmol) to afford the title compound after prep. acidic HPLC (95 mg, assume 100% yield). LCMS (METHOD 3) (ES): m/z 639.4 [M+H]⁺, RT=0.99 min.

### Preparation 109: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-methyl-pyrazole-3-carboxamide

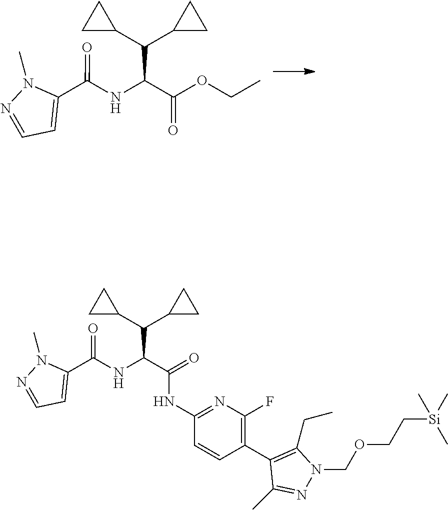 [SMILES: CCOC(=O)[C@@H](NC(=O)c1ccnn1C)C(C1CC1)C1CC1 ; CCc1c(-c2ccc(NC(=O)[C@@H](NC(=O)c3ccnn3C)C(C3CC3)C3CC3)nc2F)c(C)nn1COCC[Si](C)(C)C]

According to the method of Preparation 27 the product of Preparation 99 (60 mg, 0.196 mmol) was reacted with the product from Preparation 39 (72.3 mg, 0.206 mmol) to afford the title compound after prep. acidic HPLC (119 mg, assume 100% yield). LCMS (METHOD 3) (ES): m/z 610.4 [M+H]⁺, RT=0.98 min.

### Preparation 110: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(3-tetrahydropyran-2-yloxypropyl)pyrazole-3-carboxamide

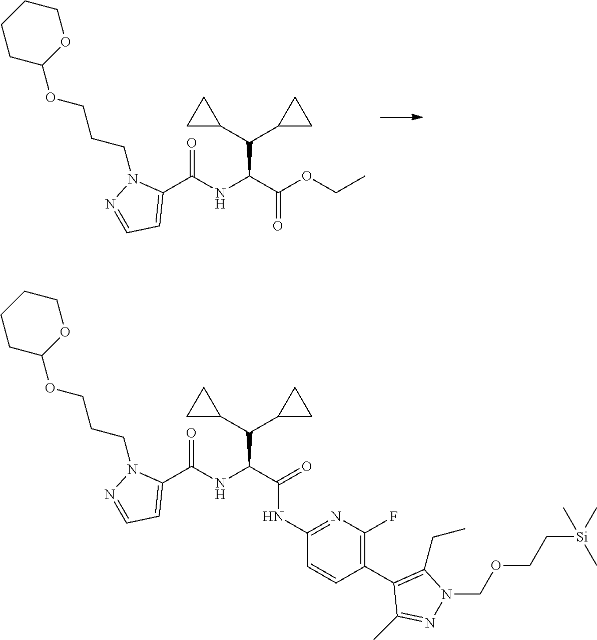 [SMILES: CCOC(=O)[C@@H](NC(=O)c1ccnn1CCCOC1CCCCO1)C(C1CC1)C1CC1 ; CCc1c(-c2ccc(NC(=O)[C@@H](NC(=O)c3ccnn3CCCOC3CCCCO3)C(C3CC3)C3CC3)nc2F)c(C)nn1COCC[Si](C)(C)C]

According to the method of Preparation 27 the product of Preparation 103 (60 mg, 0.138 mmol) was reacted with the product from Preparation 39 (51 mg, 0.145 mmol) to afford the title compound after prep. acidic HPLC (102 mg, assume 100% yield). LCMS (METHOD 3) (ES): m/z 738.4 [M+H]⁺, RT=1.06 min.

### Preparation 111: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-5-methyl-1-tetrahydropyran-4-yl-pyrazole-4-carboxamide

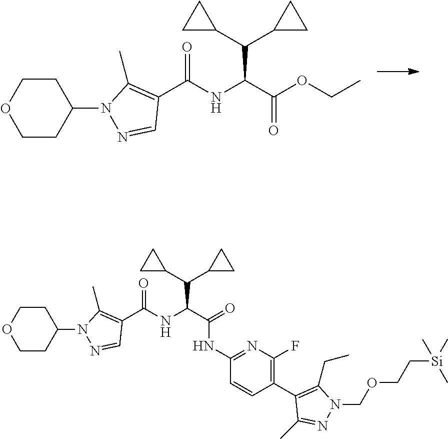 [SMILES: CCOC(=O)[C@@H](NC(=O)c1cnn(C2CCOCC2)c1C)C(C1CC1)C1CC1 ; CCc1c(-c2ccc(NC(=O)[C@@H](NC(=O)c3cnn(C4CCOCC4)c3C)C(C3CC3)C3CC3)nc2F)c(C)nn1COCC[Si](C)(C)C]

According to the method of Preparation 27 the product of Preparation 100 (60 mg, 0.154 mmol) was reacted with the product from Preparation 39 (56.7 mg, 0.162 mmol) to afford the title compound after prep. acidic HPLC (106 mg, assume 100% yield). LCMS (METHOD 3) (ES): m/z 694.4 [M+H]⁺, RT=0.97 min.

### Preparation 112: tert-butyl N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]carbamate

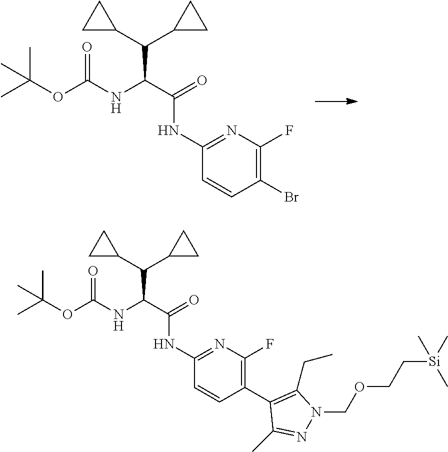 [SMILES: CC(C)(C)OC(=O)N[C@H](C(=O)Nc1ccc(Br)c(F)n1)C(C1CC1)C1CC1 ; CCc1c(-c2ccc(NC(=O)[C@@H](NC(=O)OC(C)(C)C)C(C3CC3)C3CC3)nc2F)c(C)nn1COCC[Si](C)(C)C]

According to the method of Preparation 22 the product of Preparation 90 (1.02 g, 2.31 mmol) was reacted with the product from Preparation 6 (1.01 g, 2.77 mmol). The obtained crude compound was purified by silica column chromatography (230-400 mesh), eluting with EtOAc in heptane, to afford the title compound as a colourless solid (1.03 g, 74% yield). LCMS (METHOD 3) (ES): m/z 602.4 [M+H]⁺, RT=1.07 min.

### Preparation 113: (2S)-2-amino-3,3-dicyclopropyl-N-[5-[5-ethyl-3-methyl-1-(2-trimethylsilylethoxymethyl)pyrazol-4-yl]-6-fluoro-2-pyridyl]propenamide hydrochloride

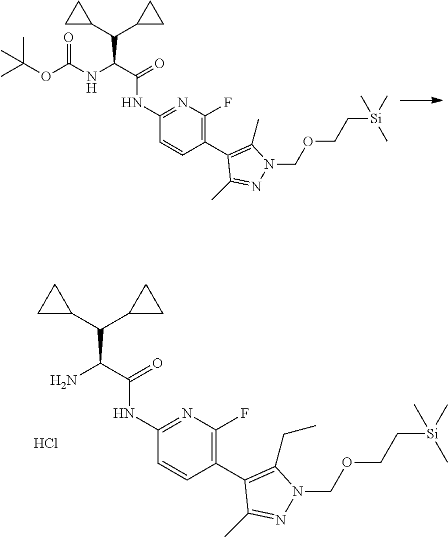 [SMILES: Cc1nn(COCC[Si](C)(C)C)c(C)c1-c1ccc(NC(=O)[C@@H](NC(=O)OC(C)(C)C)C(C2CC2)C2CC2)nc1F ; CCc1c(-c2ccc(NC(=O)[C@@H](N)C(C3CC3)C3CC3)nc2F)c(C)nn1COCC[Si](C)(C)C ; Cl]

Hydrogen chloride (3M in CPME, 2.28 mL) was added to a solution of the product from Preparation 89 (1.03 g, 1.71 mmol) in DCM (10 mL) and the reaction mixture was stirred at room temperature for 2 hours then concentrated in vacuo to leave the title compound as an off-white solid (921 g, assume 100% yield). LCMS (METHOD 3) (ES): m/z 502.2 [M+H]⁺, RT=0.74 min.

### Preparation 114: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(4,4,4-trifluoro-3-hydroxy-butyl)pyrazole-3-carboxamide (Diastereomer 1)

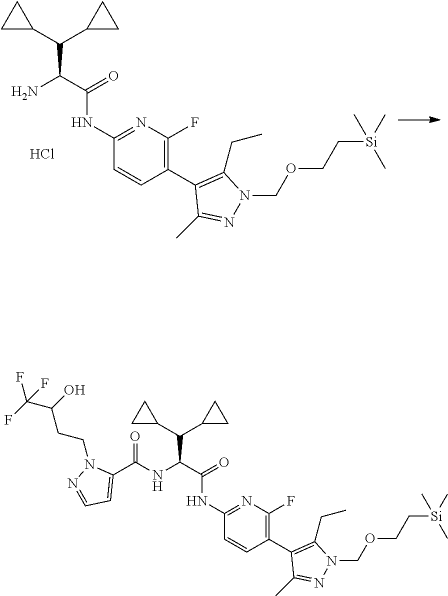 [SMILES: CCc1c(-c2ccc(NC(=O)[C@@H](N)C(C3CC3)C3CC3)nc2F)c(C)nn1COCC[Si](C)(C)C ; Cl ; CCc1c(-c2ccc(NC(=O)[C@@H](NC(=O)c3ccnn3CCC(O)C(F)(F)F)C(C3CC3)C3CC3)nc2F)c(C)nn1COCC[Si](C)(C)C]

According to the method of Preparation 11 the product of Preparation 113 (20 mg, 0.037 mmol) was reacted with the product from Preparation 87 (13.3 mg, 0.056 mmol) to afford the title compound after prep. basic HPLC (26 mg, assume 100% yield). LCMS (METHOD 3) (ES): m/z 720.3 [M−H]⁻, RT=0.99 min.

### Preparation 115: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(4,4,4-trifluoro-3-hydroxy-butyl)pyrazole-3-carboxamide (Diastereomer 2)

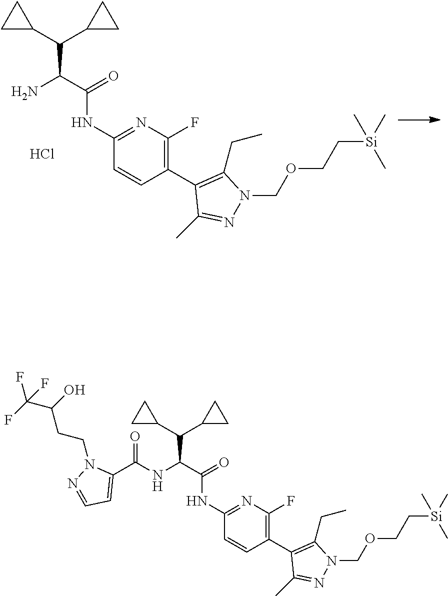 [SMILES: CCc1c(-c2ccc(NC(=O)[C@@H](N)C(C3CC3)C3CC3)nc2F)c(C)nn1COCC[Si](C)(C)C ; Cl ; CCc1c(-c2ccc(NC(=O)[C@@H](NC(=O)c3ccnn3CCC(O)C(F)(F)F)C(C3CC3)C3CC3)nc2F)c(C)nn1COCC[Si](C)(C)C]

According to the method of Preparation 11 the product of Preparation 113 (20 mg, 0.037 mmol) was reacted with the product from Preparation 88 (13.3 mg, 0.056 mmol) to afford the title compound after prep. basic HPLC (26 mg, assume 100% yield). LCMS (METHOD 3) (ES): m/z 720.3 [M−H]⁻, RT=0.99 min.

### Preparation 116: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-3-methyl-isoxazole-4-carboxamide

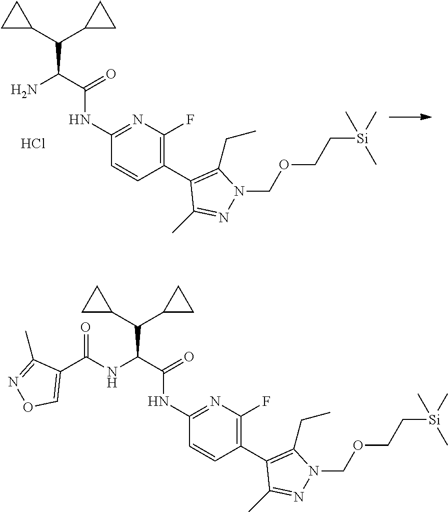 [SMILES: CCc1c(-c2ccc(NC(=O)[C@@H](N)C(C3CC3)C3CC3)nc2F)c(C)nn1COCC[Si](C)(C)C ; Cl ; CCc1c(-c2ccc(NC(=O)[C@@H](NC(=O)c3conc3C)C(C3CC3)C3CC3)nc2F)c(C)nn1COCC[Si](C)(C)C]

According to the method of Preparation 11 the product of Preparation 113 (20 mg, 0.037 mmol) was reacted with 3-methylisoxazole-4-carboxylic acid (7.1 mg, 0.056 mmol) to afford the title compound after prep. basic HPLC (22 mg, assume 100% yield). LCMS (METHOD 3) (ES): m/z 611.3 [M+H]⁺, RT=0.97 min.

### Preparation 117: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-3-ethyl-isoxazole-4-carboxamide

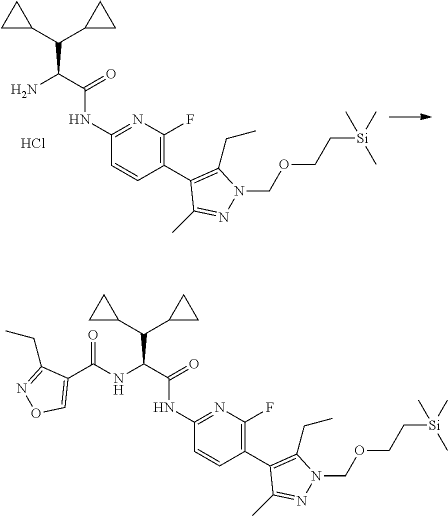

According to the method of Preparation 11 the product of Preparation 113 (20 mg, 0.037 mmol) was reacted with 3-ethylisoxazole-4-carboxylic acid (7.9 mg, 0.056 mmol) to afford the title compound after prep. basic HPLC (23 mg, assume 100% yield). LCMS (METHOD 3) (ES): m/z 625.3 [M+H]⁺, RT=1.00 min.

### Preparation 118: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-3-isopropyl-isoxazole-4-carboxamide

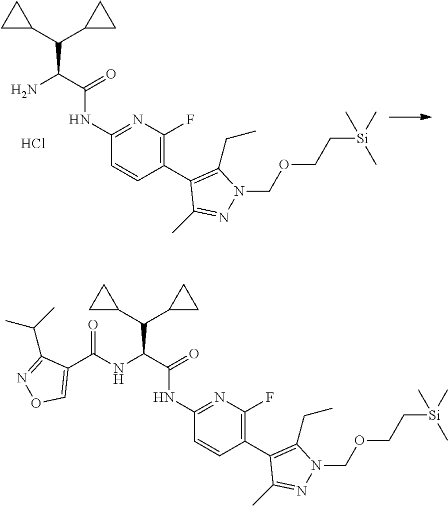

According to the method of Preparation 11 the product of Preparation 113 (20 mg, 0.037 mmol) was reacted with 3-isopropylisoxazole-4-carboxylic acid (8.6 mg, 0.056 mmol) to afford the title compound after prep. basic HPLC (24 mg, assume 100% yield). LCMS (METHOD 3) (ES): m/z 639.3 [M+H]⁺, RT=1.02 min.

### Preparation 119: 2-amino-N-(5-bromo-2-pyridyl)-3,3-dicyclopropyl-propanamide hydrochloride

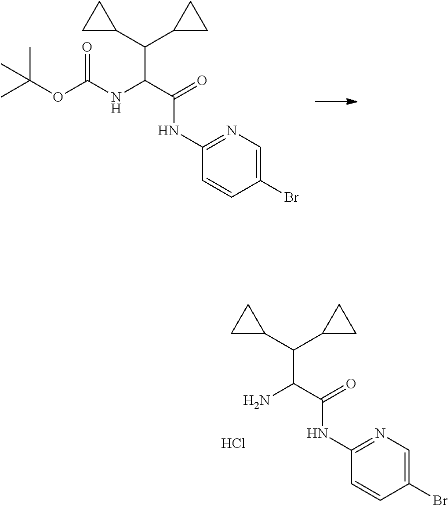 [SMILES: CC(C)(C)OC(=O)NC(C(=O)Nc1ccc(Br)cn1)C(C1CC1)C1CC1 ; Cl ; NC(C(=O)Nc1ccc(Br)cn1)C(C1CC1)C1CC1]

According to the method of Preparation 8 the product of Preparation 4 (100 mg, 0.23 mmol) was reacted to afford the title compound (80 mg, assume 100% yield). 1H NMR (300 MHz, DMSO-d6) δ 11.23 (s, 1H), 8.50 (d, J=2.20 Hz, 1H), 8.43 (br s, 3H), 8.16-7.98 (m, 2H), 4.12 (d, J=5.87 Hz, 1H), 0.88-0.78 (m, 2H), 0.74-0.63 (m, 1H), 0.0-0.52 (m, 1H), 0.50-0.35 (m, 2H), 0.3-0.21 (m, 4H), 0.13-0.11 (m, 1H); LCMS (METHOD 2) (ESI): m/z 324 [M+H]⁺; RT=1.87 min; (ACQUITY BEH C18 column, 0.05% FA in water with MeCN).

### Preparation 120: N-[1-[(5-bromo-2-pyridyl)carbamoyl]-2,2-dicyclopropyl-ethyl]-2-isopropyl-pyrazole-3-carboxamide

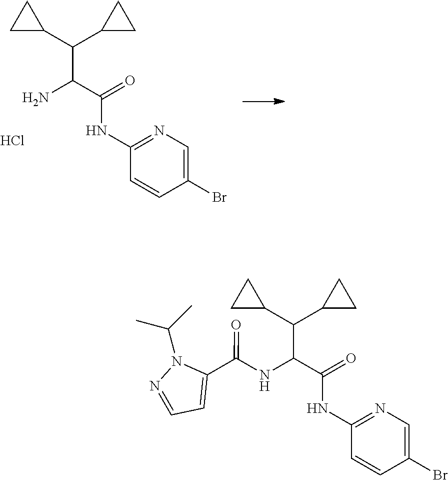 [SMILES: Cl ; NC(C(=O)Nc1ccc(Br)cn1)C(C1CC1)C1CC1 ; CC(C)n1nccc1C(=O)NC(C(=O)Nc1ccc(Br)cn1)C(C1CC1)C1CC1]

According to the method of Preparation 49 the product of Preparation 119 (100 mg, 0.27 mmol) was reacted with 2-isopropylpyrazole-3-carboxylic acid (53 mg, 0.33 mmol) to afford the title compound (110 mg, 86% yield). 1H NMR (400 MHz, DMSO-d6) δ 10.86 (s, 1H), 8.45 (dt, J=3.21, 2.53 Hz, 2H), 8.10-8.00 (m, 2H), 7.50 (d, J=1.85 Hz, 1H), 6.91 (d, J=1.96 Hz, 1H), 5.43-5.28 (m, 1H), 4.90 (t, J=8.01 Hz, 1H), 1.35 (dd, J=15.26, 6.65 Hz, 6H), 1.00-0.90 (m, 1H), 0.89-0.80 (m, 1H), 0.78-0.67 (m, 1H), 0.51-0.40 (m, 1H), 0.39-0.31 (m, 1H), 0.30-0.05 (m, 6H); LCMS (METHOD 2) (ESI): m/z 460 [M+H]⁺; RT=2.21 min (ACQUITY BEH C18 column, 0.1% FA in water with MeCN).

### Preparation 121: (2S)-2-amino-N-(6-bromo-3-pyridyl)-3,3-dicyclopropyl-propanamide hydrochloride

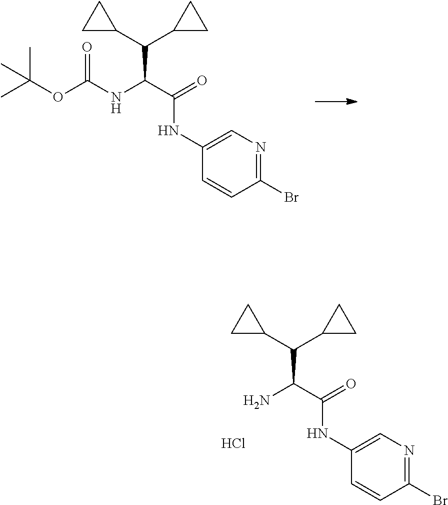 [SMILES: CC(C)(C)OC(=O)N[C@H](C(=O)Nc1ccc(Br)nc1)C(C1CC1)C1CC1 ; Cl ; N[C@H](C(=O)Nc1ccc(Br)nc1)C(C1CC1)C1CC1]

According to the method of Preparation 8 the product of Preparation 73 (205 mg, 0.48 mmol) was reacted to afford the title compound (184 mg, assume 100% yield). 1H NMR (400 MHz, DMSO-d6) δ 11.50 (s, 1H), 8.71 (d, J=2.51 Hz, 1H), 8.45 (br d, J=3.38 Hz, 3H), 8.06 (dd, J=8.66, 2.78 Hz, 1H), 7.66 (d, J=8.61 Hz, 1H), 4.17 (t, J=5.50 Hz, 1H), 0.96-0.68 (m, 3H), 0.61-0.35 (m, 3H), 0.34-0.22 (m, 4H), 0.17-0.14 (m, 1H); LCMS (METHOD 2) (ESI): m/z: 324 [M+H]⁺; RT=1.74 min (ACQUITY BEH C18 column, 0.05% FA in water with MeCN).

### Preparation 122: N-[(1S)-1-[(6-bromo-3-pyridyl)carbamoyl]-2,2-dicyclopropyl-ethyl]-2-isopropyl-pyrazole-3-carboxamide

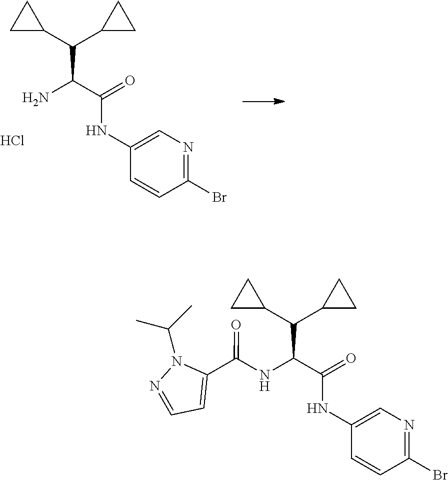 [SMILES: Cl ; N[C@H](C(=O)Nc1ccc(Br)nc1)C(C1CC1)C1CC1 ; CC(C)n1nccc1C(=O)N[C@H](C(=O)Nc1ccc(Br)nc1)C(C1CC1)C1CC1]

According to the method of Preparation 49 the product of Preparation 119 (150 mg, 0.41 mmol) was reacted with 2-isopropylpyrazole-3-carboxylic acid (80 mg, 0.50 mmol) to afford the title compound (130 mg, 68% yield). 1H NMR (400 MHz, DMSO-d6) δ 10.58 (s, 1H), 8.61 (d, J=2.62 Hz, 1H), 8.52 (d, J=8.61 Hz, 1H), 8.03 (dd, J=8.66, 2.78 Hz, 1H), 7.61 (d, J=8.72 Hz, 1H), 7.50 (d, J=1.96 Hz, 1H), 6.94 (d, J=1.96 Hz, 1H), 5.45-5.36 (m, 1H), 4.79 (t, J=8.12 Hz, 1H), 1.36 (dd, J=12.97, 6.65 Hz, 6H), 0.96-0.86 (m, 1H), 0.83-0.72 (m, 2H), 0.50-0.43 (m, 1H), 0.41-0.25 (m, 3H), 0.23-0.20 (m, 3H), 0.09-0.02 (m, 1H); LCMS (METHOD 2) (ESI): m/z: 460 [M+H]⁺; RT=2.08 min (ACQUITY BEH C18 column, 0.05% FA in water with MeCN).

### Preparation 123: tert-butyl N-[(1S)-2-[(6-bromo-3-pyridyl)amino]-1-cyclohexyl-2-oxo-ethyl]carbamate

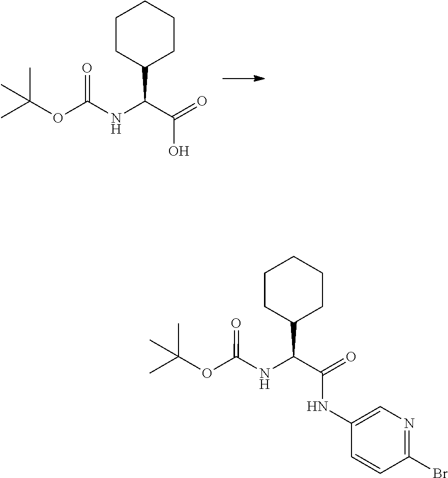 [SMILES: CC(C)(C)OC(=O)N[C@H](C(=O)O)C1CCCCC1 ; CC(C)(C)OC(=O)N[C@H](C(=O)Nc1ccc(Br)nc1)C1CCCCC1]

HATU (72.5 mg, 0.19 mmol) was added to a solution of (2S)-2-(tert-butoxycarbonylamino)-2-cyclohexyl-acetic acid (58.0 mg, 0.23 mmol), 6-bromopyridin-3-amine (30.0 mg, 0.173 mmol) and DIPEA (0.151 mL, 0.87 mmol) in DMF (0.5 mL) and stirred at 55° C. for 16 hours. The reaction mixture was diluted with EtOAc (5 mL) and washed successively with water, saturated NaHCO₃ (aq.) and brine solution then concentrated to dryness in vacuo. The crude tert-butyl N-[(1S)-2-[(6-bromo-3-pyridyl)amino]-1-cyclohexyl-2-oxo-ethyl]carbamate was used without further purification. Assumed quantitative yield. LCMS (METHOD 4) (ES): m/z 414.2 [M+H]⁻, RT=0.85 min.

### Preparation 124: (2S)-2-amino-N-(6-bromo-3-pyridyl)-2-cyclohexyl-acetamide 2,2,2-trifluoroacetic acid salt

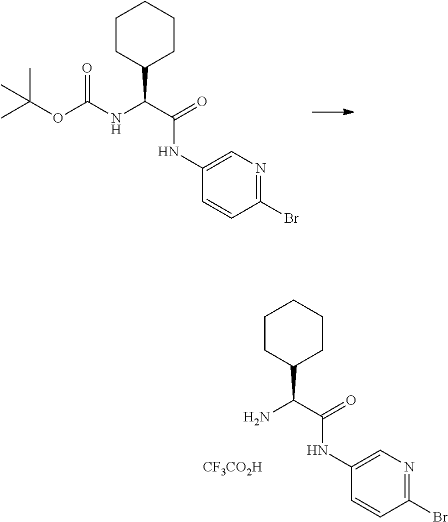 [SMILES: CC(C)(C)OC(=O)N[C@H](C(=O)Nc1ccc(Br)nc1)C1CCCCC1 ; "CF3CO2H" ; N[C@H](C(=O)Nc1ccc(Br)nc1)C1CCCCC1]

TFA (0.5 mL) was added to a solution of the product from Preparation 123 (71.5 mg, 0.173 mmol) in DCM (2 mL) at room temperature. After 30 min the reaction mixture was concentrated in vacuo to leave crude title compound, which was used without purification. Assumed quantitative yield. LCMS (METHOD 4) (ES): m/z 314.0 [M+H]⁻, RT=0.60 min.

### Preparation 125: tert-butyl N-[(1S)-2-[(6-bromo-3-pyridyl)amino]-1-cyclohexyl-2-oxo-ethyl]carbamate

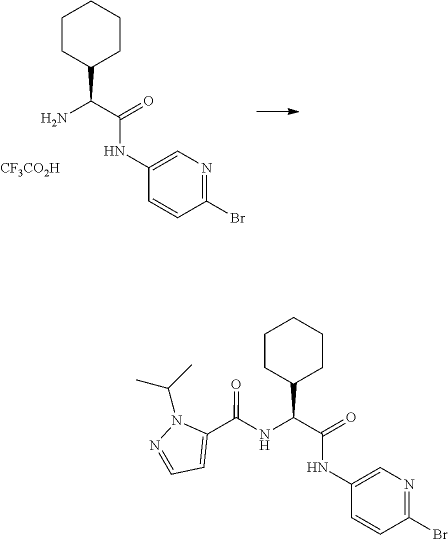 [SMILES: "CF3CO2H" ; N[C@H](C(=O)Nc1ccc(Br)nc1)C1CCCCC1 ; CC(C)n1nccc1C(=O)N[C@H](C(=O)Nc1ccc(Br)nc1)C1CCCCC1]

HATU (72.6 mg, 0.19 mmol) was added to a solution of the product from Preparation 124 (73.9 mg, 0.173 mmol), 2-isopropylpyrazole-3-carboxylic acid (34.7 mg, 0.225 mmol) and DIPEA (0.3 mL, 1.73 mmol) in DMF (0.7 mL) and stirred for 1 hour at room temperature. The reaction mixture was diluted with EtOAc (5 mL) and washed successively with water, saturated NaHCO₃ (aq.) and brine solution then concentrated to dryness in vacuo. The obtained crude compound was purified by silica column chromatography (230-400 mesh), eluting with EtOAc in heptane, to afford the title compound as a colourless solid (40.0 mg, 52% yield). 1H NMR (400 MHz, CDCl₃) δ 8.44-8.37 (m, 2H), 7.96 (dd, J=8.7, 2.8 Hz, 1H), 7.51 (d, J=2.0 Hz, 1H), 7.42 (d, J=8.6 Hz, 1H), 6.64 (d, J=8.4 Hz, 1H), 6.58 (d, J=2.0 Hz, 1H), 5.40 (h, J=6.6 Hz, 1H), 4.44 (t, J=8.2 Hz, 1H), 2.05-1.66 (m, 6H), 1.48 (dd, J=12.3, 6.6 Hz, 6H), 1.37-1.01 (m, 5H); LCMS (METHOD 4) (ES): m/z 450.3 [M+H]⁻, RT=0.80 min.

### Preparation 126: tert-butyl N-[(1S)-2-[(5-bromo-2-pyridyl)amino]-1-cyclohexyl-2-oxo-ethyl]carbamate

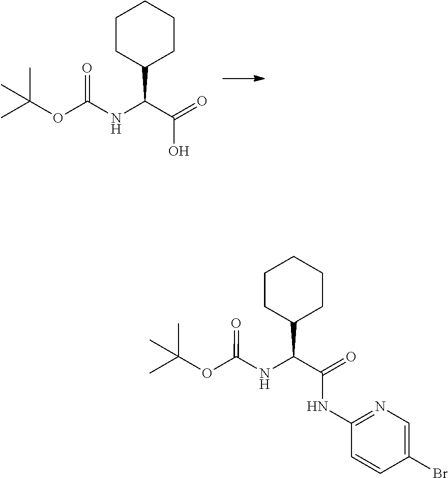 [SMILES: CC(C)(C)OC(=O)N[C@H](C(=O)O)C1CCCCC1 ; CC(C)(C)OC(=O)N[C@H](C(=O)Nc1ccc(Br)cn1)C1CCCCC1]

CDI (63.0 mg, 0.39 mmol) was added to a solution of (2S)-2-(tert-butoxycarbonylamino)-2-cyclohexyl-acetic acid (100.0 mg, 0.39 mmol) in DMF (2 mL) and stirred at room temperature for 5 minutes. To the reaction mixture was added DBU (0.058 mL, 0.39 mmol) followed by 5-bromopyridin-2-amine (67.2 mg, 0.39 mmol) and the reaction mixture was stirred at 60° C. for 48 hours. The reaction mixture was cooled to room temperature, diluted with Et₂O (20 mL) and washed successively with water, saturated NaHCO₃ (aq.) and brine solution. The organic phase was concentrated to dryness in vacuo. The crude product was used without further purification, (160.2 mg, assume 100% yield). LCMS (METHOD 3) (ES): m/z 414.2 [M+H]⁺, RT=0.92 min.

### Preparation 127: (2S)-2-amino-N-(5-bromo-2-pyridyl)-2-cyclohexyl-acetamide 2,2,2-trifluoroacetic acid salt

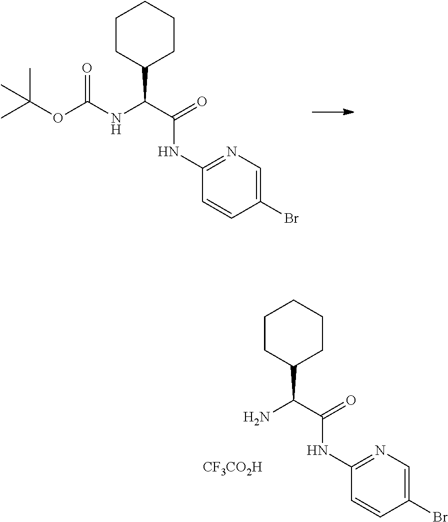 [SMILES: CC(C)(C)OC(=O)N[C@H](C(=O)Nc1ccc(Br)cn1)C1CCCCC1 ; "CF3CO2H" ; N[C@H](C(=O)Nc1ccc(Br)cn1)C1CCCCC1]

According to the method of Preparation 124 the compound of Preparation 126 (160.2 mg, 0.39 mmol) was reacted to give the title compound as an off-white solid (165.6 mg, assume 100% yield). LCMS (METHOD 3) (ES): m/z 314.2 [M+H]⁺, RT=0.53 min.

### Preparation 128: N-[(1S)-2-[(5-bromo-2-pyridyl)amino]-1-cyclohexyl-2-oxo-ethyl]-2-isopropyl-pyrazole-3-carboxamide

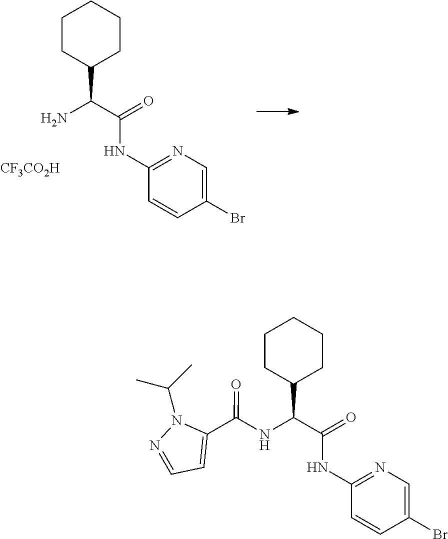 [SMILES: "CF3CO2H" ; N[C@H](C(=O)Nc1ccc(Br)cn1)C1CCCCC1 ; CC(C)n1nccc1C(=O)N[C@H](C(=O)Nc1ccc(Br)cn1)C1CCCCC1]

According to the method of Preparation 125 the compound of Preparation 127 (165.6 mg, 0.39 mmol) was reacted to give the title compound as an off-white solid (25.0 mg, 14% yield). 1H NMR (400 MHz, DMSO-d6) δ 10.79 (s, 1H), 8.49-8.39 (m, 2H), 8.07 (d, J=8.9 Hz, 1H), 8.01 (dd, J=9.1, 2.5 Hz, 1H), 7.49 (d, J=1.9 Hz, 1H), 6.93 (d, J=1.9 Hz, 1H), 5.36 (p, J=6.6 Hz, 1H), 4.51 (t, J=8.2 Hz, 1H), 1.91-1.49 (m, 6H), 1.34 (dd, J=10.3, 6.6 Hz, 6H), 1.20 (dd, J=23.2, 11.8 Hz, 5H); LCMS (METHOD 3) (ES): m/z 450.3 [M+H]⁺, RT=0.86 min.

### Preparation 129: tert-butyl N-[(1S)-2-amino-1-((1r,4S)-4-methylcyclohexyl)-2-oxo-ethyl]carbamate

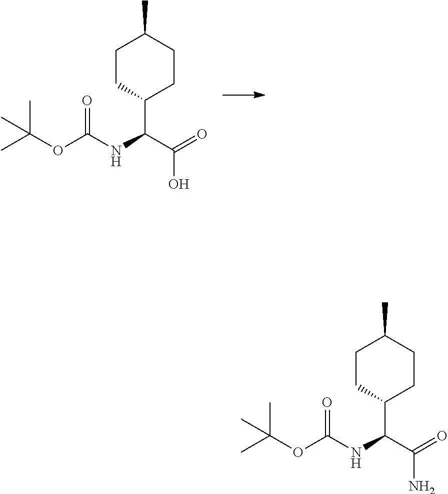 [SMILES: CC(C)(C)OC(=O)N[C@H](C(=O)O)[C@H]1CC[C@H](C)CC1 ; CC(C)(C)OC(=O)N[C@H](C(N)=O)[C@H]1CC[C@H](C)CC1]

HATU (210 mg, 0.55 mmol) was added to a solution of (S)-2-((tert-butoxycarbonyl)amino)-2-((1r,4S)-4-methylcyclohexyl)acetic acid (synthesis described in WO2018229079, 100 mg, 0.36 mmol) in DMF (3 mL). The reaction mixture was cooled to 0° C. whereupon NH₄Cl (97 mg, 1.84 mmol) and DIPEA (0.41 mL, 1.84 mmol) were added. The reaction mixture was stirred at room temperature for 16 hours. The reaction mixture was quenched with water (15 mL). The resulting precipitate was collected by filtration and dried under vacuum to afford the title compound as a yellow solid (60.0 mg, 60% yield). 1H NMR (400 MHz, CDCl₃) δ 7.25 (br s, 1H), 6.97 (br s, 1H), 6.51 (d, J=9.2 Hz, 1H), 3.73-3.70 (br t, J=6.8 Hz, 1H), 1.66-1.49 (m, 4H), 1.37 (s, 9H) 1.23-1.21 (m, 1H), 1.05-0.98 (m, 2H), 0.84-0.82 (m, 6H); LCMS (METHOD 2) (ES): m/z 271 [M+H]⁺; RT=1.65 min (ACQUITY BEH C18 column, 0.05% FA in water with MeCN).

### Preparation 130: tert-butyl N-[(1S)-2-[(5-bromo-2-pyridyl)amino]-1-((1r,4S)-4-methylcyclohexyl)-2-oxo-ethyl]carbamate

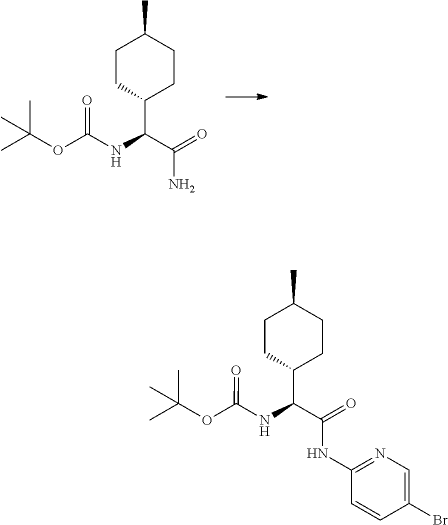 [SMILES: CC(C)(C)OC(=O)N[C@H](C(N)=O)[C@H]1CC[C@H](C)CC1 ; CC(C)(C)OC(=O)N[C@H](C(=O)Nc1ccc(Br)cn1)[C@H]1CC[C@H](C)CC1]

Cs₂CO₃ (240 mg, 0.74 mmol) was added to a solution of the product from Preparation 129 (100 mg, 0.37 mmol) and 5-bromo-2-iodopyridine (104 mg, 0.37 mmol). The reaction mixture was purged with argon for 15 minutes before the addition of Pd(PPh₃)₄ (21.0 mg, 0.018 mmol) and Xantphos (21.0 mg, 0.037 mmol). The reaction mixture was stirred at 110° C. for 1 hour. The cooled reaction mixture was filtered through Celite™ washing the pad with EtOAc (50 mL). The filtrate was dried over Na₂SO₄, filtered and concentrated in vacuo. The obtained crude compound was purified by silica column chromatography (230-400 mesh), eluting with EtOAc (30-50%) in heptane, to afford the title compound as a pale yellow solid (80.0 mg, 50% yield). 1H NMR (300 MHz, DMSO-d6) δ 10.56 (s, 1H), 8.44 (d, J=1.83 Hz, 1H), 8.09-7.94 (m, 2H), 6.96 (d, J=8.44 Hz, 1H), 4.06 (t, J=7.52 Hz, 1H), 1.74-1.11 (m, 16H), 0.91-0.73 (m, 6H); LCMS (METHOD 2) (ES): m/z: 426 [M+H]⁺; RT=2.92 min (ACQUITY BEH C18 column, 0.05% FA in water with MeCN).

### Preparation 131: (2S)-2-amino-N-(5-bromo-2-pyridyl)-2-(4-methylcyclohexyl)acetamide hydrochloride

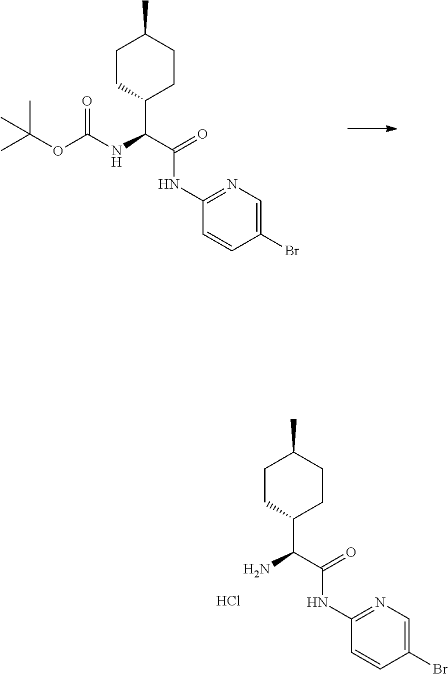 [SMILES: CC(C)(C)OC(=O)N[C@H](C(=O)Nc1ccc(Br)cn1)[C@H]1CC[C@H](C)CC1 ; Cl ; C[C@H]1CC[C@H]([C@H](N)C(=O)Nc2ccc(Br)cn2)CC1]

Hydrogen chloride (4M solution in dioxane, 3.0 mL) was added to a solution of the product from Preparation 130 (40.0 mg, 0.09 mmol) in 1,4-dioxane (1.0 mL) at 0° C. The reaction mixture was stirred to room temperature over 30 minutes, then concentrated in vacuo to afford the title compound as a tan solid (30.0 mg, 88% yield). LCMS (METHOD 2) (ES): m/z: 326 [M+H]⁺; RT=1.52 min; (ACQUITY BEH C18 column, 0.1% FA in water with MeCN).

### Preparation 132: N-[(1S)-2-[(5-bromo-2-pyridyl)amino]-1-((1r,4S)-4-methylcyclohexyl)-2-oxo-ethyl]-2-isopropyl-pyrazole-3-carboxamide

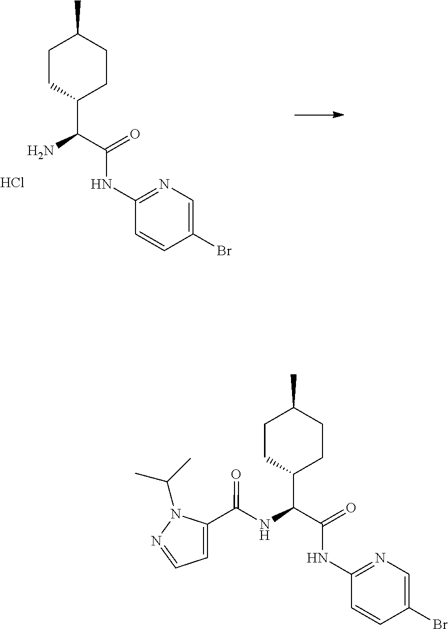 [SMILES: Cl ; C[C@H]1CC[C@H]([C@H](N)C(=O)Nc2ccc(Br)cn2)CC1 ; CC(C)n1nccc1C(=O)N[C@H](C(=O)Nc1ccc(Br)cn1)[C@H]1CC[C@H](C)CC1]

HATU (45.0 mg, 0.11 mmol) was added to a solution of the product from Preparation 131 (30.0 mg, 0.09 mmol) in DMF (2 mL). The reaction mixture was cooled to 0° C. whereupon 2-isopropylpyrazole-3-carboxylic acid (14.7 mg, 0.09 mmol) and DIPEA (0.09 mL, 0.46 mmol) were added. The reaction mixture was stirred at room temperature for 16 hours. The reaction mixture was quenched with water (20 mL) and extracted with EtOAc (2×20 mL). The combined organic layers were dried over Na₂SO₄, filtered and concentrated in vacuo. The obtained crude compound was purified by silica column chromatography (230-400 mesh), eluting with EtOAc (10%) in heptane, to afford the title compound as an off-white solid (30.0 mg, 70% yield). LCMS (METHOD 2) (ES): m/z 462 [M+H]⁺; RT=2.35 min (ACQUITY BEH C18 column, 0.1% FA in water with MeCN).

### Preparation 133: tert-butyl N-[(1S)-2-[(6-bromo-3-pyridyl)amino]-1-((1r,4S)-4-methylcyclohexyl)-2-oxo-ethyl]carbamate

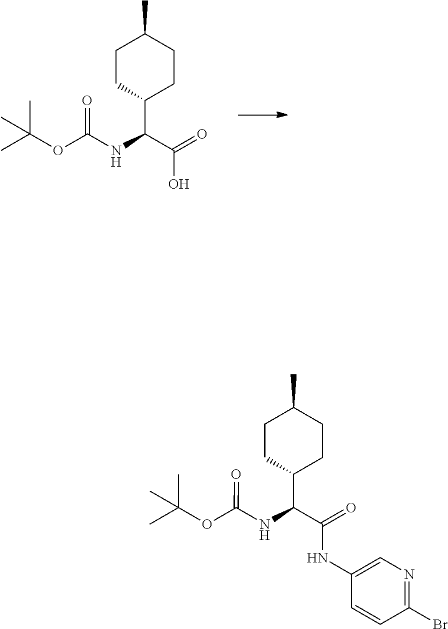 [SMILES: CC(C)(C)OC(=O)N[C@H](C(=O)O)[C@H]1CC[C@H](C)CC1 ; CC(C)(C)OC(=O)N[C@H](C(=O)Nc1ccc(Br)nc1)[C@H]1CC[C@H](C)CC1]

According to the method of Preparation 123 (S)-2-((tert-butoxycarbonyl)amino)-2-((1r,4S)-4-methylcyclohexyl)acetic acid (200 mg, 0.73 mmol) was reacted to give the title compound as an off-white solid (180 mg, 57% yield). 1H NMR (400 MHz, DMSO-d6) δ 10.37 (s, 1H), 8.60 (d, J=2.62 Hz, 1H), 8.01 (dd, J=8.72, 2.72 Hz, 1H), 7.59 (d, J=8.72 Hz, 1H), 7.01 (d, J=8.39 Hz, 1H), 3.91 (t, J=7.85 Hz, 1H), 1.79-1.45 (m, 4H), 1.40-1.35 (m, 9H), 1.31-1.20 (m, 1H), 1.15-0.94 (m, 2H), 0.91-0.75 (m, 6H); LCMS (METHOD 2) (ES): m/z: 426 [M+H]⁺; RT=2.32 min (ACQUITY BEH C18 column, 0.1% FA in water with MeCN).

### Preparation 134: (2S)-2-amino-N-(6-bromo-3-pyridyl)-2-((1r,4S)-4-methylcyclohexyl)acetamide hydrochloride

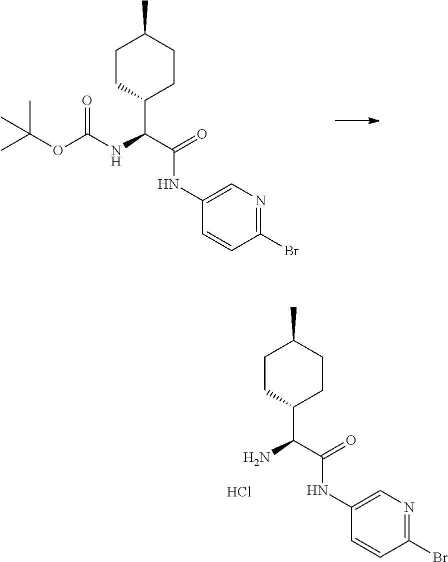 [SMILES: CC(C)(C)OC(=O)N[C@H](C(=O)Nc1ccc(Br)nc1)[C@H]1CC[C@H](C)CC1 ; Cl ; C[C@H]1CC[C@H]([C@H](N)C(=O)Nc2ccc(Br)nc2)CC1]

According to the method of Preparation 131 the product from Preparation 133 (180 mg, 0.22 mmol) was reacted to give the title compound as an off-white solid (150 mg, 98% yield). 1H NMR (400 MHz, DMSO-d6) δ 11.63 (s, 1H), 8.74 (d, J=2.62 Hz, 1H), 8.47 (br d, J=4.25 Hz, 3H), 8.08 (dd, J=8.66, 2.78 Hz, 1H), 7.65 (d, J=8.61 Hz, 1H), 3.95 (t, J=5.45 Hz, 1H), 1.86-1.58 (m, 4H), 1.34-1.18 (m, 4H), 1.17-1.05 (m, 1H), 0.93-0.79 (m, 4H); LCMS (METHOD 2) (ES): m/z: 326 [M+H]⁺; RT=1.82 min (ACQUITY BEH C18 column, 0.05% FA in water with MeCN).

### Preparation 135: N-[(1S)-2-[(6-bromo-3-pyridyl)amino]-1-((1r,4S)-4-methylcyclohexyl)-2-oxo-ethyl]-2-isopropyl-pyrazole-3-carboxamide

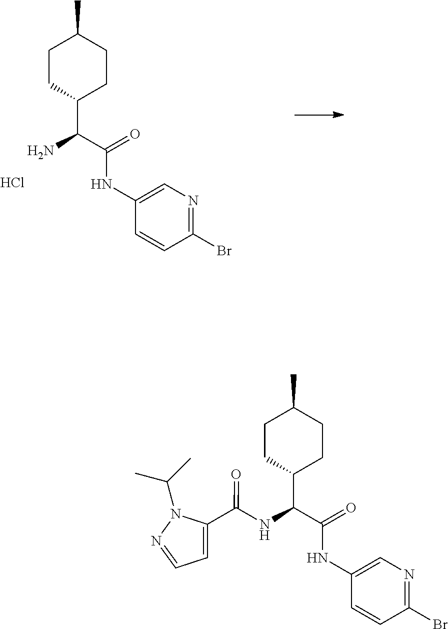 [SMILES: Cl ; C[C@H]1CC[C@H]([C@H](N)C(=O)Nc2ccc(Br)nc2)CC1 ; CC(C)n1nccc1C(=O)N[C@H](C(=O)Nc1ccc(Br)nc1)[C@H]1CC[C@H](C)CC1]

According to the method of Preparation 132 the product from Preparation 134 (150 mg, 0.22 mmol) was reacted to give the title compound as an off-white solid (120 mg, 94% yield). 1H NMR (400 MHz, DMSO-d6) δ 10.57 (s, 1H), 8.63 (d, J=2.62 Hz, 1H), 8.56 (d, J=7.96 Hz, 1H), 8.03 (dd, J=8.66, 2.78 Hz, 1H), 7.60 (d, J=8.61 Hz, 1H), 7.49 (d, J=1.85 Hz, 1H), 6.96 (d, J=1.96 Hz, 1H), 5.39-5.36 (m, 1H), 4.35 (t, J=8.39 Hz, 1H), 1.94-1.75 (m, 2H), 1.71-1.64 (m, 2H), 1.60-1.50 (m, 1H), 1.42-1.10 (m, 9H), 0.94 (d, J=6.54 Hz, 1H), 0.85 (d, J=6.43 Hz, 4H); LCMS (METHOD 2) (ES): m/z: 462 [M+H]⁺; RT=2.21 min (ACQUITY BEH C18 column, 0.1% FA in water with MeCN).

### Preparation 136: N-[(1S)-1-[[5-bromo-6-(trifluoromethyl)-2-pyridyl]carbamoyl]-2,2-dicyclopropyl-ethyl]-2-ethyl-pyrazole-3-carboxamide

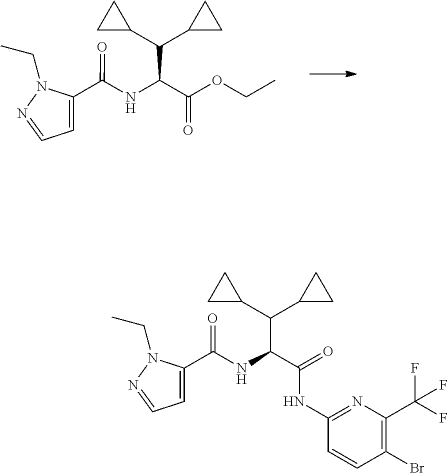

According to the method of Preparation 27 the compound of Preparation 44 (50 mg, 0.157 mmol) was reacted with 5-bromo-6-(trifluoromethyl)pyridine-2-amine (39.6 mg, 0.164 mmol) to afford the title compound after prep. acidic HPLC (56 mg, 69% yield). LCMS (METHOD 3) (ES): m/z 515.2 [M−H]⁻, RT=0.90 min.

### Preparation 137: N-[(1S)-1-(dicyclopropylmethyl)-2-[[5-[3,5-dimethyl-2-(2-trimethylsilylethoxymethyl)-1,5-dihydropyrazol-4-yl]-6-(trifluoromethyl)-2-pyridyl]amino]-2-oxo-ethyl]-2-ethyl-pyrazole-3-carboxamide

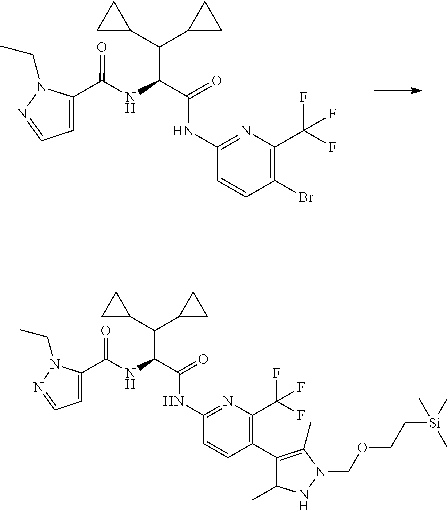 [SMILES: CCn1nccc1C(=O)N[C@H](C(=O)Nc1ccc(Br)c(C(F)(F)F)n1)C(C1CC1)C1CC1 ; CCn1nccc1C(=O)N[C@H](C(=O)Nc1ccc(C2=C(C)N(COCC[Si](C)(C)C)NC2C)c(C(F)(F)F)n1)C(C1CC1)C1CC1]

According to the method of Preparation 22 the compound of Preparation 136 (56 mg, 0.11 mmol) was reacted with the compound of Preparation 21 (42.0 mg, 0.12 mmol) to afford the title compound after prep. acidic HPLC (4.0 mg, 5.6% yield). LCMS (METHOD 3) (ES): m/z 658.4 [M−H]⁻, RT=1.00 min.

### Preparation 138: tert-butyl N-[(1S)-1-[(5-bromo-6-chloro-2-pyridyl)carbamoyl]-2,2-dicyclopropyl-ethyl]carbamate

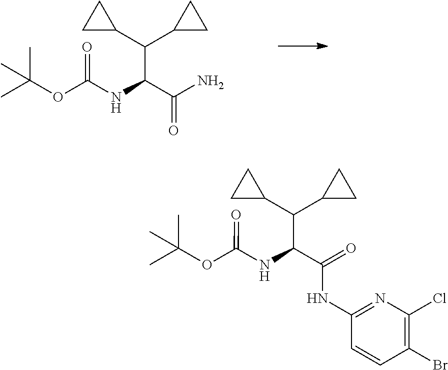 [SMILES: CC(C)(C)OC(=O)N[C@H](C(N)=O)C(C1CC1)C1CC1 ; CC(C)(C)OC(=O)N[C@H](C(=O)Nc1ccc(Br)c(Cl)n1)C(C1CC1)C1CC1]

According to the method of Preparation 90 the compound of Preparation 89 (500 mg, 1.9 mmol) was reacted with 3,6-dibromo-2-chloropyridine (530 mg, 2.0 mmol) to afford the title compound as a pale yellow solid (715 mg, 84% yield) after purification by silica column chromatography (230-400 mesh), eluting with EtOAc (0-50%) in heptane. 1H NMR (400 MHz, DMSO-d6) δ 10.90 (s, 1H), 8.19 (d, J=8.7 Hz, 1H), 8.00 (d, J=8.7 Hz, 1H), 7.03-6.53 (m, 1H), 4.56-4.22 (m, 1H), 1.39 (s, 9H), 0.98-0.71 (m, 2H), 0.60-0.01 (m, 9H); LCMS (METHOD 3) (ES): m/z 458.2 [M−H]⁻, RT=0.95 min.

### Preparation 139: tert-butyl N-[(1S)-1-[[6-chloro-5-[3,5-dimethyl-1-(2-trimethylsilylethoxymethyl)pyrazol-4-yl]-2-pyridyl]carbamoyl]-2,2-dicyclopropyl-ethyl]carbamate

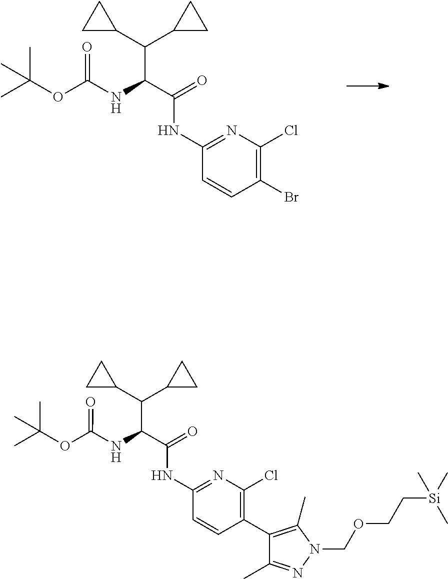 [SMILES: CC(C)(C)OC(=O)N[C@H](C(=O)Nc1ccc(Br)c(Cl)n1)C(C1CC1)C1CC1 ; Cc1nn(COCC[Si](C)(C)C)c(C)c1-c1ccc(NC(=O)[C@@H](NC(=O)OC(C)(C)C)C(C2CC2)C2CC2)nc1Cl]

According to the method of Preparation 22 the compound of Preparation 138 (250 mg, 0.54 mmol) was reacted with the compound of Preparation 21 (230 mg, 0.64 mmol) to afford the title compound after prep. acidic HPLC (170 mg, 51% yield). 1H NMR (400 MHz, DMSO-d6) δ 10.81 (s, 1H), 8.09 (d, J=8.4 Hz, 1H), 7.75 (d, J=8.3 Hz, 1H), 6.96 (d, J=8.9 Hz, 1H), 5.45-5.21 (m, 2H), 4.39 (t, J=7.6 Hz, 1H), 3.61-3.49 (m, 2H), 2.13 (s, 3H), 2.01 (s, 3H), 1.40 (s, 9H), 1.02-0.72 (m, 4H), 0.65-0.03 (m, 9H), −0.04 (s, 9H). LCMS (METHOD 3) (ES): m/z 606.4 [M+H]⁺, RT=1.05 min.

### Preparation 140: (2S)-2-amino-N-[6-chloro-5-[3,5-dimethyl-1-(2-trimethylsilylethoxymethyl)pyrazol-4-yl]-2-pyridyl]-3,3-dicyclopropyl-propanamide hydrochloride

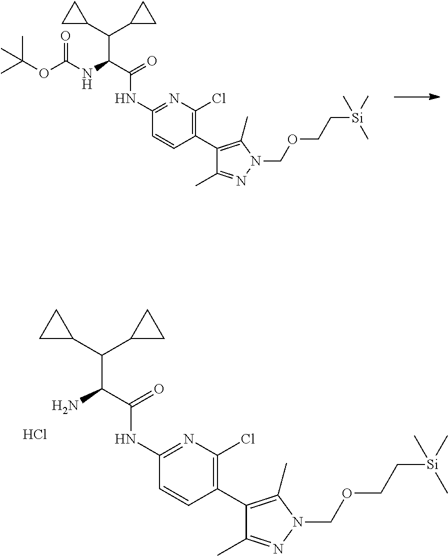 [SMILES: Cc1nn(COCC[Si](C)(C)C)c(C)c1-c1ccc(NC(=O)[C@@H](NC(=O)OC(C)(C)C)C(C2CC2)C2CC2)nc1Cl ; Cl ; Cc1nn(COCC[Si](C)(C)C)c(C)c1-c1ccc(NC(=O)[C@@H](N)C(C2CC2)C2CC2)nc1Cl]

According to the method of Preparation 8 the compound of Preparation 139 (170 mg, 0.28 mmol) was reacted to afford the crude title compound (152 mg, assume 100% yield). LCMS (METHOD 3) (ES): m/z 506.3 [M+H]⁺, RT=0.82 min.

### Preparation 141: N-[(1S)-1-[[6-chloro-5-[3,5-dimethyl-1-(2-trimethylsilylethoxymethyl)pyrazol-4-yl]-2-pyridyl]carbamoyl]-2,2-dicyclopropyl-ethyl]-2-methyl-pyrazole-3-carboxamide

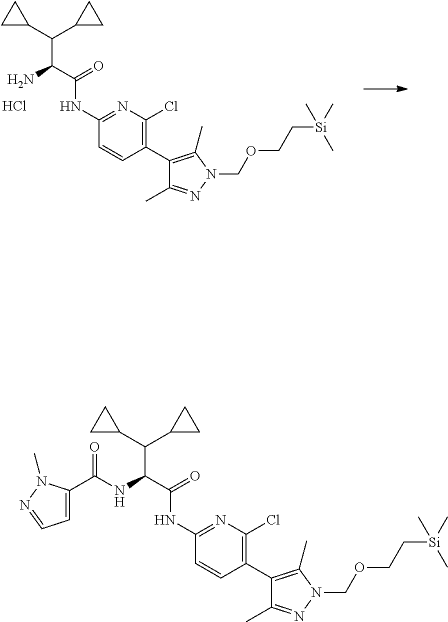 [SMILES: Cc1nn(COCC[Si](C)(C)C)c(C)c1-c1ccc(NC(=O)[C@@H](N)C(C2CC2)C2CC2)nc1Cl ; Cl ; Cc1nn(COCC[Si](C)(C)C)c(C)c1-c1ccc(NC(=O)[C@@H](NC(=O)c2ccnn2C)C(C2CC2)C2CC2)nc1Cl]

According to the method of Preparation 11 the compound of Preparation 140 (24 mg, 0.047 mmol) was reacted with 2-methylpyrazole-3-carboxylic acid (7.1 mg, 0.056 mmol) to afford the title compound after prep. acidic HPLC (16 mg, 55% yield). LCMS (METHOD 3) (ES): m/z 612.4 [M+H]⁺, RT=0.97 min.

### Preparation 142: N-[(1S)-1-[[6-chloro-5-[3,5-dimethyl-1-(2-trimethylsilylethoxymethyl)pyrazol-4-yl]-2-pyridyl]carbamoyl]-2,2-dicyclopropyl-ethyl]-2-ethyl-pyrazole-3-carboxamide

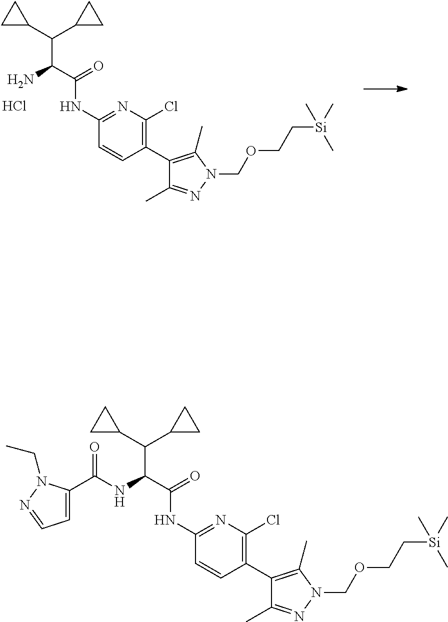

According to the method of Preparation 11 the compound of Preparation 140 (24 mg, 0.047 mmol) was reacted with 2-ethylpyrazole-3-carboxylic acid (7.9 mg, 0.056 mmol) to afford the title compound after prep. acidic HPLC (17 mg, 58% yield). LCMS (METHOD 3) (ES): m/z 626.4 [M+H]⁺, RT=0.99 min.

### Preparation 143: N-[(1S)-1-[[6-chloro-5-[3,5-dimethyl-1-(2-trimethylsilylethoxymethyl)pyrazol-4-yl]-2-pyridyl]carbamoyl]-2,2-dicyclopropyl-ethyl]-2-isopropyl-pyrazole-3-carboxamide

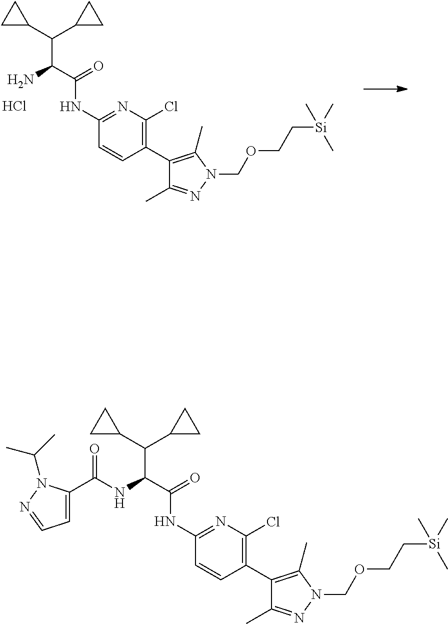

According to the method of Preparation 11 the compound of Preparation 140 (24 mg, 0.047 mmol) was reacted with 2-isopropylpyrazole-3-carboxylic acid (8.7 mg, 0.056 mmol) to afford the title compound after prep. acidic HPLC (12 mg, 38% yield). LCMS (METHOD 3) (ES): m/z 640.4 [M+H]⁺, RT=1.02 min.

### Preparation 144: N-[(1S)-1-[[6-chloro-5-[3,5-dimethyl-1-(2-trimethylsilylethoxymethyl)pyrazol-4-yl]-2-pyridyl]carbamoyl]-2,2-dicyclopropyl-ethyl]-3-methyl-isoxazole-4-carboxamide

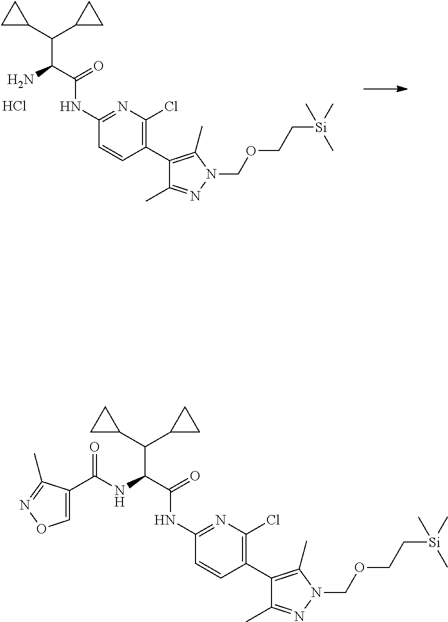 [SMILES: Cc1nn(COCC[Si](C)(C)C)c(C)c1-c1ccc(NC(=O)[C@@H](N)C(C2CC2)C2CC2)nc1Cl ; Cl ; Cc1nocc1C(=O)N[C@H](C(=O)Nc1ccc(-c2c(C)nn(COCC[Si](C)(C)C)c2C)c(Cl)n1)C(C1CC1)C1CC1]

According to the method of Preparation 11 the compound of Preparation 140 (24 mg, 0.047 mmol) was reacted with 3-methylisoxazole-4-carboxylic acid (7.1 mg, 0.056 mmol) to afford the title compound after prep. acidic HPLC (6 mg, 21% yield). LCMS (METHOD 3) (ES): m/z 613.4 [M+H]⁺, RT=0.97 min.

### Preparation 145: N-[(1S)-1-[[6-chloro-5-[3,5-dimethyl-1-(2-trimethylsilylethoxymethyl)pyrazol-4-yl]-2-pyridyl]carbamoyl]-2,2-dicyclopropyl-ethyl]-3-ethyl-isoxazole-4-carboxamide

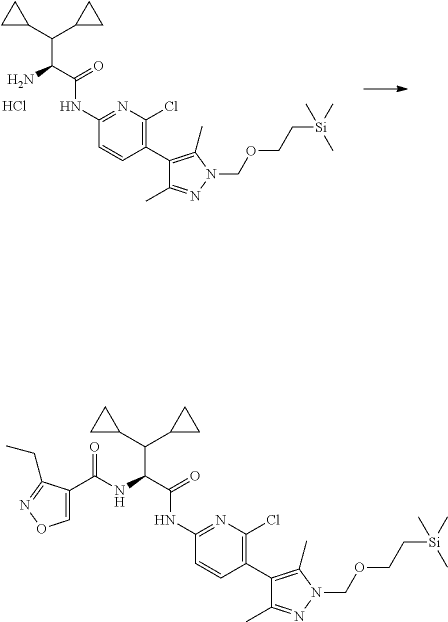

According to the method of Preparation 11 the compound of Preparation 140 (24 mg, 0.047 mmol) was reacted with 3-ethylisoxazole-4-carboxylic acid (8.0 mg, 0.056 mmol) to afford the title compound after prep. acidic HPLC (17 mg, 57% yield). LCMS (METHOD 3) (ES): m/z 627.4 [M+H]⁺, RT=1.00 min.

### Preparation 146: N-[(1S)-1-[[6-chloro-5-[3,5-dimethyl-1-(2-trimethylsilylethoxymethyl)pyrazol-4-yl]-2-pyridyl]carbamoyl]-2,2-dicyclopropyl-ethyl]-3-isopropyl-isoxazole-4-carboxamide

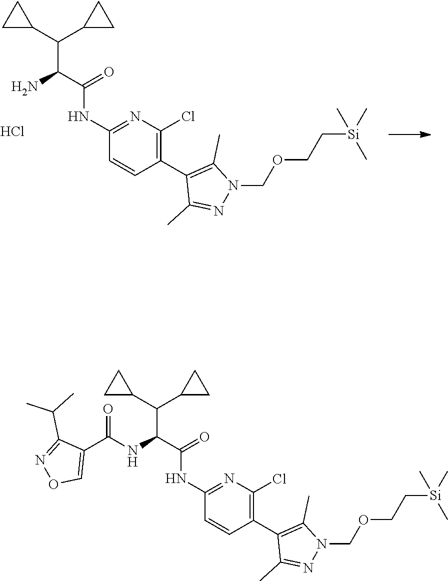

According to the method of Preparation 11 the compound of Preparation 140 (24 mg, 0.047 mmol) was reacted with 3-isopropylisoxazole-4-carboxylic acid (8.7 mg, 0.056 mmol) to afford the title compound after prep. acidic HPLC (18 mg, 60% yield). LCMS (METHOD 3) (ES): m/z 641.4 [M+H]⁺, RT=1.02 min.

### Preparation 147: ethyl 2-(2-methylsulfanylethyl)pyrazole-3-carboxylate

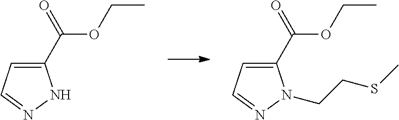

DIAD (17.0 mL, 85.7 mmol) was added slowly to a solution of ethyl 1H-pyrazole-5-carboxylate (10.0 g, 71.4 mmol) and triphenylphosphine (20.0 g, 78.6 mmol) in anhydrous THF (150 mL) at 0° C. 2-Methylsulfanylethanol (7.20 g, 78.6 mmol) was added and the reaction mixture was stirred at room temperature for 18 hours. The reaction mixture was concentrated in vacuo and the obtained crude compound was purified by silica column chromatography (230-400 mesh), eluting with EtOAc in heptane, to afford the title compound as a brown oil (3.30 g, 21% yield). 1H NMR (400 MHz, CDCl₃) δ 7.59 (d, J=2.2 Hz, 1H), 6.88 (d, J=2.2 Hz, 1H), 4.69 (t, J=7.1 Hz, 2H), 4.31 (q, J=6.9 Hz, 2H), 2.86 (t, J=7.1 Hz, 2H), 2.03 (s, 3H), 1.30 (t, J=7.1 Hz, 3H); LCMS (METHOD 2) (ESI): m/z: 215 [M+H]⁺; 88%; RT=1.86 min (ACQUITY BEH C18 column, 0.1% FA in water with MeCN)

### Preparation 148: 2-(2-methylsulfanylethyl)pyrazole-3-carboxylic acid

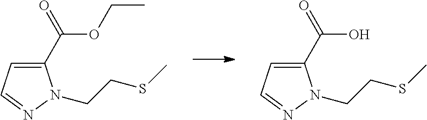

LiOH·H₂O (1.17 g, 28.0 mmol) was added to a solution of the compound of Preparation 147 (2.0 g, 9.34 mmol) in THF:H₂O (10 mL, 1:1) and stirred at room temperature for 12 hours. The reaction mixture was cooled to 0° C. and the pH was adjusted to -3 with hydrogen chloride (5M aqueous solution). The resultant solid was filtered and dried in vacuo to leave the title compound (900 mg, 51% yield). 1H NMR (600 MHz, CDCl₃) δ 13.39 (br s, 1H), 7.55 (d, J=2.0 Hz, 1H), 6.82 (d, J=1.9 Hz, 1H), 4.78-4.61 (m, 2H), 2.92-2.76 (m, 2H), 2.03 (s, 3H); LCMS (METHOD 2) (ESI): m/z: 187 [M+H]⁺; 99%; RT=1.79 min (ACQUITY BEH C18 column, 0.05% FA in water with MeCN).

### Preparation 149: ethyl 2-(2-methylsulfinylethyl)pyrazole-3-carboxylate

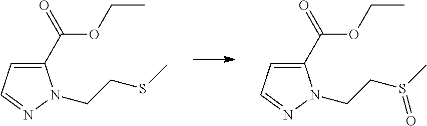

Sodium periodate (5.9 g, 28.0 mmol) was added to a solution of the compound of Preparation 147 (5.0 g, 23.4 mmol) in EtOH:H₂O (20 mL, 1:1) at 0° C. The reaction mixture was stirred to room temperature over 16 hours. The reaction mixture was quenched with H₂O (200 mL) and the mixture was extracted with EtOAc (2×200 mL) The combined extracts were dried over Na₂SO₄, filtered and concentrated in vacuo to afford the title compound as an off-white solid (2.50 g, 58% yield). 1H NMR (400 MHz, DMSO-d6) δ 7.62 (d, J=2.0 Hz, 1H), 6.91 (d, J=2.1 Hz, 1H), 4.94-4.83 (m, 2H), 4.31 (q, J=7.1 Hz, 2H), 3.35-3.27 (m, 1H), 3.17-3.06 (m, 1H), 2.56 (s, 3H), 1.31 (t, J=7.1 Hz, 3H); LCMS (METHOD 2) (ESI): m/z: 231 [M+H]⁺; 99%; RT=2.96 min (Xbridge C18 column, 5 mM Ammonium Bicarbonate in water with MeCN)

### Preparation 150: 2-(2-methylsulfinylethyl)pyrazole-3-carboxylic acid

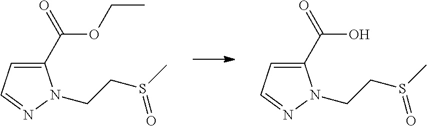

LiO·H₂O (4.1 g, 97.8 mmol) was added to a solution of the compound of Preparation 149 (7.5 g, 32.6 mmol) in THF:H₂O (60 mL, 1:1) and stirred at room temperature for 12 hours. The reaction mixture was diluted with H₂O (50 mL), cooled to 0° C. and the pH was adjusted to ˜4 with hydrogen chloride (5M aqueous solution). The mixture was extracted with DCM/MeOH (9:1, 2×250 mL). The combined extracts were dried over Na₂SO₄, filtered and concentrated in vacuo to afford the title compound as an off-white solid (5.50 g, 83% yield). 1H NMR (400 MHz, DMSO-d6) δ 7.28 (d, J=1.7 Hz, 1H), 6.41 (d, J=1.8 Hz, 1H), 5.00-4.82 (m, 2H), 3.29-3.19 (m, 1H), 3.14-3.07 (m, 1H), 2.55 (s, 3H); LCMS (METHOD 2) (ESI): m/z: 203 [M+H]⁺; 98%; RT=1.96 min (ACQUITY BEH C18 column, 0.05% TFA in water with MeCN).

### Preparation 151: ethyl 2-(2-methylsulfonylethyl)pyrazole-3-carboxylate

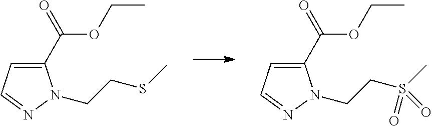 [SMILES: CCOC(=O)c1ccnn1CCSC ; CCOC(=O)c1ccnn1CCS(C)(=O)=O]

MCPBA (12.9 g 74.8 mmol) was added to a solution of the compound from Preparation 147 (4.0 g, 18.7 mmol) in DCM (60 mL) at 0° C. The reaction mixture was stirred at room temperature for 16 hours. The reaction mixture was cooled to 0° C. and basified to pH 10 with saturated aq. NaHCO₃, diluted with H₂O (100 mL) and extracted with DCM (2×100 mL). The combined organic layers were dried over Na₂SO₄, filtered and concentrated in vacuo and the obtained crude compound was purified by silica column chromatography (230-400 mesh), eluting with EtOAc in heptane, to afford the title compound as a colourless oil (3.0 g, 66% yield). 1H NMR (400 MHz, DMSO-d6) δ 7.53 (d, J=2 Hz, 1H), 6.88 (d, J=2 Hz, 1H), 5.04 (t, J=6.8 Hz, 2H), 4.41 (q, J=14 Hz, 2H), 3.60 (t, J=7.2 Hz, 2H), 2.87 (s, 3H), 1.40 (t, J=7.2 Hz, 3H); LCMS (METHOD 2) (ESI): m/z: 247 [M+H]⁺; 96%; RT=1.80 min (ACQUITY BEH C18 column, 0.05% FA in water with MeCN).

### Preparation 152: 2-(2-methylsulfonylethyl)pyrazole-3-carboxylic acid

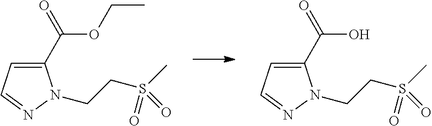 [SMILES: CCOC(=O)c1ccnn1CCS(C)(=O)=O ; CS(=O)(=O)CCn1nccc1C(=O)O]

According to the method of Preparation 148 the compound of Preparation 151 (8.0 g, 54.8 mmol) was reacted to afford the crude title compound (5.3 g, 75% yield). 1H NMR (400 MHz, DMSO-d6) δ 13.50 (s, 1H) 7.60 (d, J=2.0 Hz, 1H), 6.85 (d, J=2.0 Hz, 1H), 4.95 (t, J=7.2 Hz, 2H), 3.68 (t, J=7.2 Hz, 2H), 2.95 (s, 3H); LCMS (METHOD 2) (ESI): m/z: 219 [M+H]⁺; 98%; RT=1.26 min (ACQUITY BEH C18 column, 0.05% FA in water with MeCN).

### Preparation 153: ethyl 2-(3-methylsulfanylpropyl)pyrazole-3-carboxylate

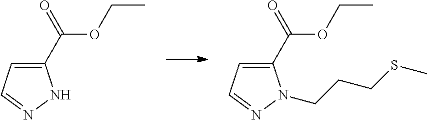

According to the method of Preparation 147, ethyl 1H-pyrazole-5-carboxylate (1.0 g, 7.14 mmol) was reacted with 3-methylsulfanylpropan-1-ol (832 mg, 7.86 mmol) to afford the title compound as an off-white solid (1.0 g, 62% yield). 1H NMR (300 MHz, DMSO-d6) δ 7.57 (d, J=2.0 Hz, 1H), 6.88 (d, J=2.0 Hz, 1H), 4.56 (t, J=6.9 Hz, 2H), 4.30 (q, J=6.9 Hz, 2H), 2.43 (t, J=7.1 Hz, 2H), 2.03 (m, 5H), 1.31 (t, J=7.1 Hz, 3H); LCMS (METHOD 2) (ESI): m/z: 229 [M+H]⁺; 99%; RT=1.97 min (ACQUITY BEH C18 column, 0.1% FA in water with MeCN).

### Preparation 154: 2-(3-methylsulfanylpropyl)pyrazole-3-carboxylic acid

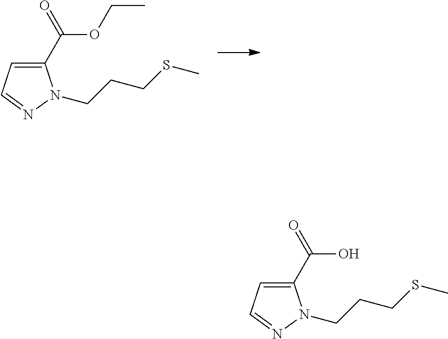

According to the method of Preparation 148 the compound of Preparation 153 (1.0 g, 4.38 mmol) was reacted to afford the crude title compound (600 mg, 69% yield). 1H NMR (300 MHz, DMSO-d6) δ 13.34 (br s, 1H), 7.53 (d, J=1.8 Hz, 1H), 6.82 (d, J=1.8 Hz, 1H), 4.57 (t, J=7.0 Hz, 2H), 2.46-2.38 (m, 2H), 2.06-1.93 (m, 5H); LCMS (METHOD 2) (ESI): m/z: 201[M+H]⁺; 97%; RT=2.37 min (ACQUITY BEH C18 column, 0.05% FA in water with MeCN).

### Preparation 155: ethyl 2-(3-methylsulfinylpropyl)pyrazole-3-carboxylate

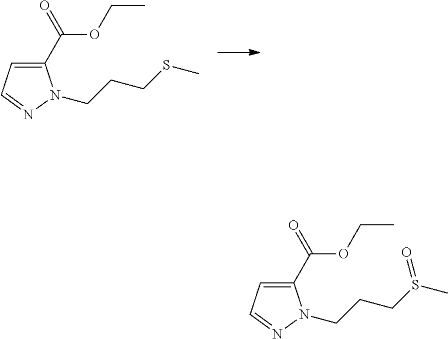 [SMILES: CCOC(=O)c1ccnn1CCCSC ; CCOC(=O)c1ccnn1CCCS(C)=O]

According to the method of Preparation 149, the compound of Preparation 153 (250 mg, 1.09 mmol) was reacted to afford the title compound as an off-white solid (250 mg, 93% yield). 1H NMR (400 MHz, DMSO-d6) δ 7.59 (d, J=2.1 Hz, 1H), 6.90 (d, J=2.1 Hz, 1H), 4.61 (t, J=7.0 Hz, 2H), 4.31 (q, J=7.1 Hz, 2H), 2.81-2.69 (m, 1H), 2.65-2.55 (m, 1H), 2.51 (s, 3H), 2.13 (quin, J=7.3 Hz, 2H), 1.31 (t, J=7.1 Hz, 3H); LCMS (METHOD 2) (ESI): m/z: 245 [M+H]⁺; 97%; RT=1.73 min (ACQUITY BEH C18 column, 0.05% FA in water with MeCN).

### Preparation 156: 2-(3-methylsulfinylpropyl)pyrazole-3-carboxylic acid

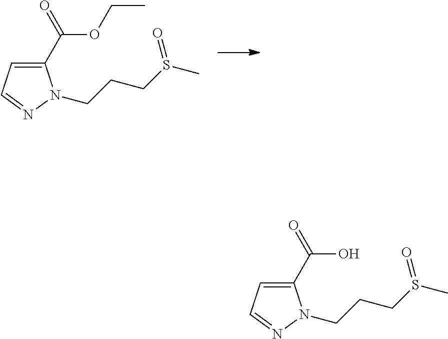 [SMILES: CCOC(=O)c1ccnn1CCCS(C)=O ; CS(=O)CCCn1nccc1C(=O)O]

According to the method of Preparation 148, the compound of Preparation 155 (250 mg, 1.02 mmol) was reacted to afford the title compound as an off-white solid (200 mg, 90% yield). 1H NMR (300 MHz, DMSO-d6) δ 13.53-13.23 (m, 1H), 7.54 (d, J=1.8 Hz, 1H), 6.82 (d, J=1.8 Hz, 1H), 4.61 (t, J=7.0 Hz, 2H), 2.78-2.56 (m, 2H), 2.51 (s, 3H), 2.18-1.93 (m, 2H); LCMS (METHOD 2) (ESI): m/z: 217 [M+H]⁺; 92%; RT=1.70 min (ACQUITY BEH C18 column, 0.05% TFA in water with MeCN).

### Preparation 157: ethyl 2-(3-methylsulfonylpropyl)pyrazole-3-carboxylate

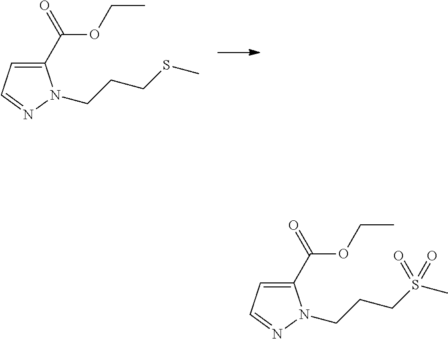 [SMILES: CCOC(=O)c1ccnn1CCCSC ; CCOC(=O)c1ccnn1CCCS(C)(=O)=O]

According to the method of Preparation 151, the compound of Preparation 153 (9.0 g, 39.5 mmol) was reacted to afford the title compound as an off-white solid (8.0 g, 78% yield). 1H NMR (300 MHz, DMSO-d6) δ 7.60 (d, J=1.8 Hz, 1H), 6.91 (d, J=1.8 Hz, 1H), 4.60 (t, J=6.9 Hz, 2H), 4.33 (d, =14.1 Hz, 2H), 3.12 (t, =10.2 Hz, 2H) 2.97 (s, 3H) 2.21-2.16 (m, 2H) 1.31 (t, J=6.9 Hz 3H); LCMS (METHOD 2) (ESI): m/z: 261 [M+H]⁺; 90%; RT=1.88 min (ACQUITY BEH C18 column, 0.05% FA in water with MeCN).

### Preparation 158: 2-(3-methylsulfonylpropyl)pyrazole-3-carboxylic acid

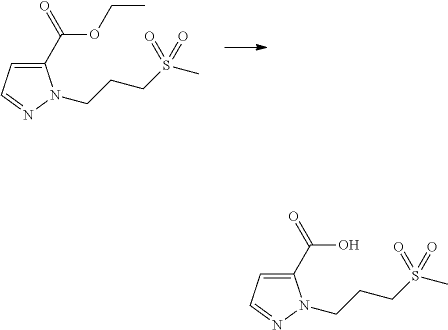 [SMILES: CCOC(=O)c1ccnn1CCCS(C)(=O)=O ; CS(=O)(=O)CCCn1nccc1C(=O)O]

According to the method of Preparation 148, the compound of Preparation 157 (7.0 g, 26.9 mmol) was reacted to afford the title compound as an off-white solid (5.3 g, 85% yield). 1H NMR (400 MHz, DMSO-d6) δ 13.41 (s, 1H) 7.56 (d, J=2.0 Hz, 1H), 6.84 (d, J=2.0 Hz, 1H), 4.61 (t, J=6.8 Hz, 2H), 3.081 (q, J=10.4 Hz, 2H), 2.97 (s, 3H), 2.22-2.15 (m, 2H); LCMS (METHOD 2) (ESI): m/z: 233 [M+H]⁺; 97%; RT=1.48 min (ACQUITY BEH C18 column, 0.05% TFA in water with MeCN).

### Preparation 159: 2-(benzyloxycarbonylamino)-3,3-dicyclopropyl-propanoic acid

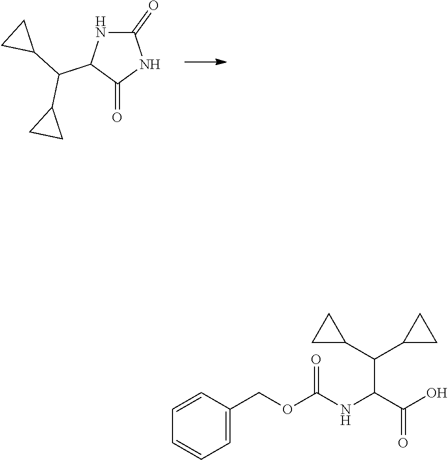 [SMILES: O=C1NC(=O)C(C(C2CC2)C2CC2)N1 ; O=C(NC(C(=O)O)C(C1CC1)C1CC1)OCc1ccccc1]

NaOH (4M aq. solution, 250 mL) was added to a suspension of the intermediate compound of Preparation 3, 5-(dicyclopropylmethyl)imidazolidine-2,4-dione (25 g, 128.8 mmol) in H₂O (1 L) and the reaction mixture was stirred at 120° C. for 16 hours. The reaction mixture was cooled to room temperature. Benzyl carbonochloridate (28.0 g, 170 mmol) was added and the reaction mixture was stirred for a further 16 hours. The reaction mixture was concentrated to low volume under reduced pressure, cooled to 0° C. and the pH was adjusted to -3 with hydrogen chloride (5M aqueous solution). The mixture was extracted with EtOAc (3×200 mL). The combined extracts were washed with H₂O (200 mL), brine solution (200 mL), dried over Na₂SO₄, filtered and concentrated in vacuo to afford the title compound as an off-white solid after trituration with pentane. (32.0 g, 82% yield) 1H NMR (400 MHz, DMSO-d6) δ 12.5 (br, s, 1H), 7.42-7.20 (m, 6H), 5.09-5.01 (q, J=12.4 Hz 2H), 4.19-4.16 (q, J=4.4 Hz 1H), 0.97-0.95 (m, 1H), 0.80-0.78 (m, 1H), 0.553-0.087 (m, 9H); LCMS (METHOD 2) (ESI): m/z: 304 [M+H]⁺; 97%; RT=2.38 min (ACQUITY BEH C18 column, 0.05% TFA in water with MeCN).

### Preparation 160: methyl (2S)-2-(benzyloxycarbonylamino)-3,3-dicyclopropyl-propanoate

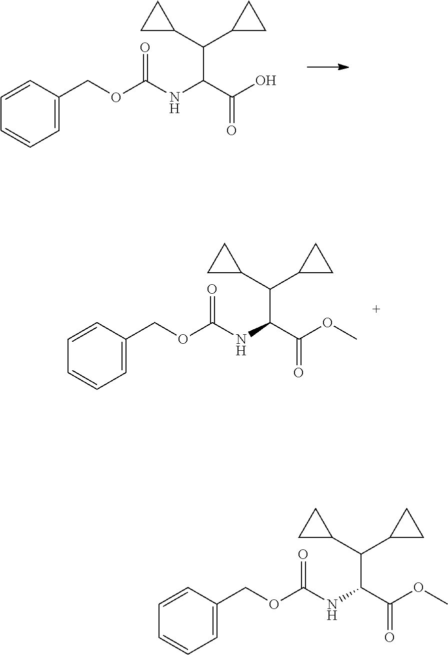 [SMILES: O=C(NC(C(=O)O)C(C1CC1)C1CC1)OCc1ccccc1 ; COC(=O)[C@@H](NC(=O)OCc1ccccc1)C(C1CC1)C1CC1 ; COC(=O)[C@H](NC(=O)OCc1ccccc1)C(C1CC1)C1CC1]

Thionyl chloride (75.9 g, 643 mmol) was added dropwise over 20 minutes to a solution of the compound of Preparation 159 (65 g, 214 mmol) in MeOH (650 mL) at 0° C. The reaction mixture was warmed to room temperature over 16 hours. The reaction mixture was concentrated under reduced pressure, diluted with saturated aq. NaHCO₃ (500 mL) and extracted with EtOAc (3×500 mL). The combined extracts were washed with H₂O (200 mL), brine solution (200 mL), dried over Na₂SO₄, filtered and concentrated in vacuo. The obtained crude compound was purified by silica gel (100-200 mesh) column chromatography (10% EtOAC/n-Hexane as eluent) to afford methyl 2-(((benzyloxy)carbonyl)amino)-3,3-dicyclopropylpropanoate as an off-white solid (50 g, 73%). The mixture of isomers were separated by SFC to afford methyl (S)-2-(((benzyloxy)carbonyl)amino)-3,3-dicyclopropylpropanoate, (24 g, 35.8%) and methyl (R)-2-(((benzyloxy)carbonyl)amino)-3,3-dicyclopropylpropanoate, (23 g, 33%) as colourless liquids.

Methyl (S)-2-(((benzyloxy)carbonyl)amino)-3,3-dicyclopropylpropanoate (160a): 1H NMR (400 MHz, CDCl₃-d6) δ 7.37-7.31 (m, 5H), 5.5 (d, J=6 Hz, 1H), 5.12 (s, 2H), 4.61-4.58 (dd, J=3.2 Hz, J=6 Hz, 1H), 3.7 (s, 3H), 0.73-0.69 (m, 3H), 0.68-0.49 (m, 4H), 0.38-0.08 (m, 4H). LCMS (METHOD 2) (ESI): m/z: 318 [M+H]⁺; 97%; RT=2.22 min (ACQUITY BEH C18 (50 mm×2.1 mm) column, 0.1% Formic acid in water, 0.1% Formic acid in MeCN). Chiral purity: 99%; RT: 3.15 min, Column: CHIRALPAK IF (250×4.6 mm) 5 μm; Co-solvent: Methanol, Total flow: 3 mL/min, % of co solvent: 15%, ABPR: 100 bar, Temperature: 30° C.

Methyl (R)-2-(((benzyloxy)carbonyl)amino)-3,3-dicyclopropylpropanoate (160b): 1H NMR (400 MHz, CDCl₃-d6) δ 7.37-7.31 (m, 5H), 5.5 (d, J=6 Hz, 1H), 5.12 (s, 2H), 4.61-4.58 (dd, J=3.2 Hz, J=6 Hz, 1H), 3.7 (s, 3H), 0.73-0.70 (m, 3H), 0.68-0.49 (m, 4H), 0.38-0.17 (m, 4H). LCMS (METHOD 2) (ESI): m/z: 318 [M+H]⁺; 98%; RT=2.60 min (ACQUITY BEH C18 (50 mm×2.1 mm) column, 0.1% Formic acid in water, 0.1% Formic acid in MeCN). Chiral purity: 99%; RT: 4.50 min, Column: CHIRALPAK IF (250×4.6 mm) 5 μm; Co-solvent: Methanol, Total flow: 3 mL/min, % of co solvent: 15%, ABPR: 100 bar, Temperature: 30° C.

### Preparation 161: methyl (2S)-2-amino-3,3-dicyclopropyl-propanoate

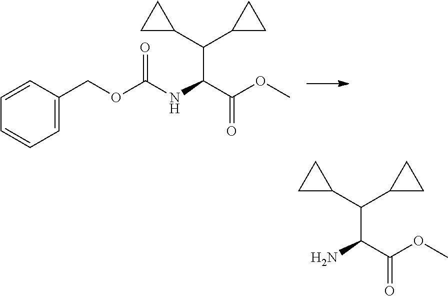 [SMILES: COC(=O)[C@@H](NC(=O)OCc1ccccc1)C(C1CC1)C1CC1 ; COC(=O)[C@@H](N)C(C1CC1)C1CC1]

Pd/C (10%, 150 mg) was added to a solution of the compound of Preparation 160a (400 mg, 1.26 mmol) in MeOH (10 mL) and placed under hydrogen at atmospheric pressure. After 3 hours the catalyst was filtered off, washing with MeOH, and the filtrate was concentrated in vacuo to give the title compound (200 mg, 86%) as an off-white tacky solid. 1H NMR (400 MHz, DMSO-d6) δ 3.60 (s, 3H), 3.46 (d, J=3.7 Hz, 1H), 1.83 (br s, 2H), 0.96-0.71 (m, 2H), 0.53-0.11 (m, 7H), 0.07-−0.15 (m, 2H).

### Preparation 162: methyl (2S)-3,3-dicyclopropyl-2-[(2-propylpyrazole-3-carbonyl)amino]propanoate

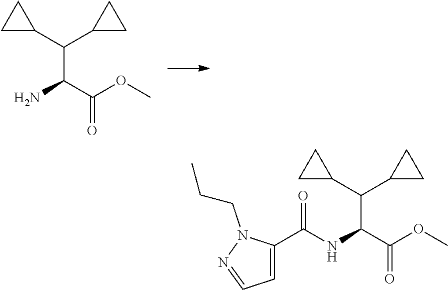 [SMILES: COC(=O)[C@@H](N)C(C1CC1)C1CC1 ; CCCn1nccc1C(=O)N[C@H](C(=O)OC)C(C1CC1)C1CC1]

According to the method of Preparation 11 the compound of Preparation 161 (160 mg, 0.87 mmol) was reacted with 2-propylpyrazole-3-carboxylic acid (148 mg, 0.97 mmol) to give the title compound as an off-white solid (240 mg, 85% yield). 1H NMR (400 MHz, CDCl₃) δ 7.48 (d, J=2.1 Hz, 1H), 6.71 (br d, J=8.6 Hz, 1H), 6.57 (d, J=2.0 Hz, 1H), 4.96 (dd, J=3.2, 8.7 Hz, 1H), 4.58-4.44 (m, 2H), 3.77 (s, 3H), 1.86 (sxt, J=7.4 Hz, 2H), 0.90 (t, J=7.5 Hz, 3H), 0.82-0.68 (m, 3H), 0.61-0.44 (m, 4H), 0.32-0.17 (m, 4H); LCMS (METHOD 2) (ESI): m/z: 320 [M+H]⁺; 97%; RT=2.38 min (ACQUITY BEH C18 column, 0.05% FA in water with MeCN).

### Preparation 163: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-propyl-pyrazole-3-carboxamide

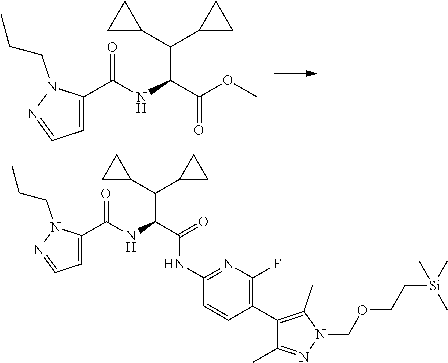

According to the method of Preparation 27 the compound of Preparation 162 (120 mg, 0.36 mmol) was reacted with the compound from Preparation 41 (132 mg, 0.394 mmol) to afford the title compound as an off-white solid (80 mg, 34% yield). LCMS (METHOD 2) (ESI): m/z: 624 [M+H]⁺; 73%; RT=2.87 min (ACQUITY BEH C18 column, 0.05% FA in water with MeCN).

### Preparation 164: methyl (2S)-3,3-dicyclopropyl-2-[[2-(2-methylsulfanylethyl)pyrazole-3-carbonyl]amino]propanoate

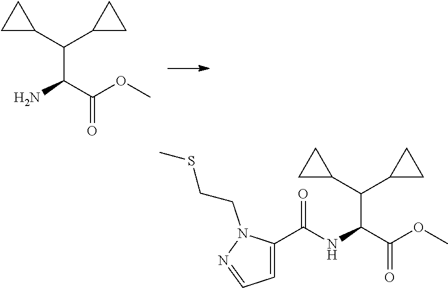

According to the method of Preparation 11 the compound of Preparation 161 (170 mg, 0.93 mmol) was reacted with the compound from Preparation 148 (155 mg, 1.03 mmol) to give the title compound as an off-white solid (220 mg, 67% yield). 1H NMR (400 MHz, CDCl₃) δ 7.51 (d, J=2.1 Hz, 1H), 6.77 (br d, J=8.6 Hz, 1H), 6.59 (d, J=2.1 Hz, 1H), 4.96 (dd, J=3.1, 8.7 Hz, 1H), 4.76 (dt, J=1.4, 7.1 Hz, 2H), 3.78 (s, 3H), 2.93 (t, J=7.1 Hz, 2H), 2.08 (s, 3H), 0.80-0.70 (m, 3H), 0.61-0.44 (m, 4H), 0.31-0.17 (m, 4H); LCMS (METHOD 2) (ESI): m/z: 352 [M+H]⁺; 99%; RT=2.56 min (ACQUITY BEH C18 column, 0.1% FA in water with MeCN).

### Preparation 165: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(2-methylsulfanylethyl)pyrazole-3-carboxamide

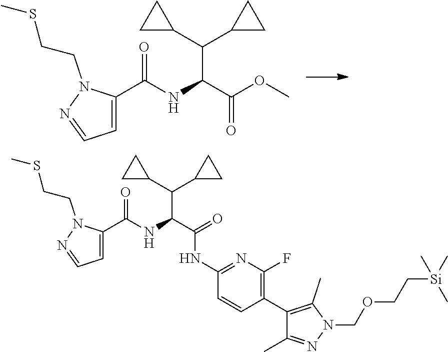

According to the method of Preparation 27 the compound of Preparation 164 (80 mg, 0.227 mmol) was reacted with the compound from Preparation 41 (93 mg, 0.25 mmol) to afford the title compound as an off-white solid (60 mg, 40% yield). LCMS (METHOD 2) (ESI): m/z: 656 [M+H]⁺; 90%; RT=2.89 min (ACQUITY BEH C18 column, 0.05% FA in water with MeCN).

### Preparation 166: methyl (2S)-3,3-dicyclopropyl-2-[[2-(2-methylsulfinylethyl)pyrazole-3-carbonyl]amino]propanoate

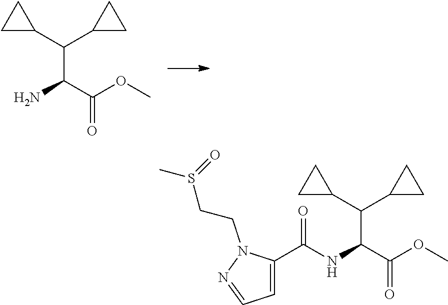

According to the method of Preparation 11 the compound of Preparation 161 (300 mg, 1.63 mmol) was reacted with the compound from Preparation 150 (364 mg, 1.80 mmol) to give the title compound as a yellow oil (500 mg, 83% yield). 1H NMR (400 MHz, CDCl₃) δ=7.59-7.50 (m, 1H), 6.89 (br t, J=8.8 Hz, 1H), 6.62 (dd, J=2.1, 2.8 Hz, 1H), 5.06-4.90 (m, 3H), 3.78 (s, 3H), 3.41-3.19 (m, 2H), 2.60 (d, J=5.3 Hz, 3H), 0.83-0.67 (m, 3H), 0.62-0.38 (m, 4H), 0.34-0.11 (m, 4H); LCMS (METHOD 2) (ESI): m/z: 368 [M+H]⁺; 96%; RT=1.96 min (ACQUITY BEH C18 column, 0.05% FA in water with MeCN).

### Preparation 167: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(2-methylsulfinylethyl)pyrazole-3-carboxamide

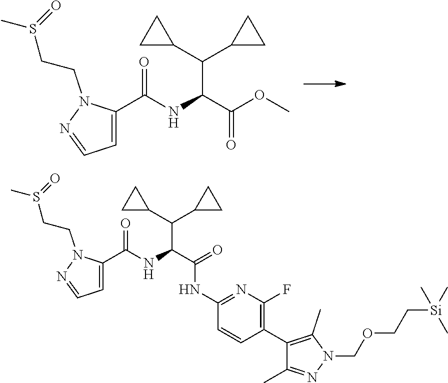

According to the method of Preparation 27 the compound of Preparation 166 (100 mg, 0.272 mmol) was reacted with the compound from Preparation 41 (102 mg, 0.299 mmol) to afford the title compound as an off-white solid (25 mg, 13% yield). LCMS (METHOD 2) (ESI): m/z: 673 [M+H]⁺; 98%; RT=4.93 min (ACQUITY BEH C18 column, 0.1% FA in water with MeCN).

### Preparation 168: methyl (2S)-3,3-dicyclopropyl-2-[[2-(2-methylsulfonylethyl)pyrazole-3-carbonyl]amino]propanoate

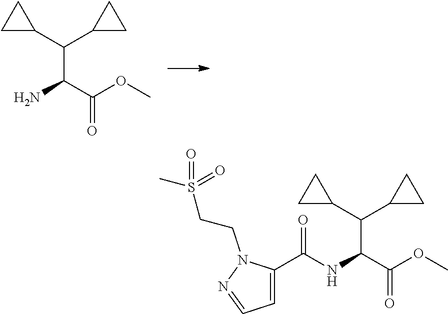

According to the method of Preparation 11 the compound of Preparation 161 (180 mg, 0.98 mmol) was reacted with the acid of Preparation 152 (235 mg, 1.08 mmol) to give the title compound as an off-white solid (100 mg, 26% yield). LCMS (METHOD 2) (ESI): m/z: 384 [M+H]⁺; 95%; RT=2.13 min (ACQUITY BEH C18 column, 0.1% FA in water with MeCN).

### Preparation 169: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(2-methylsulfonylethyl)pyrazole-3-carboxamide

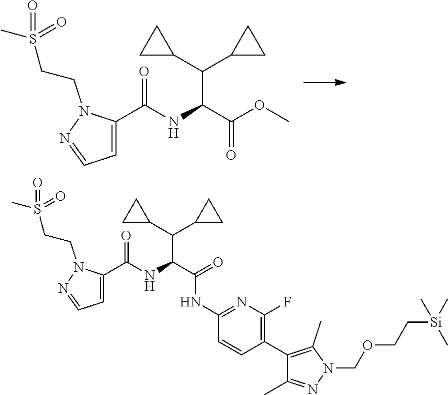

According to the method of Preparation 27 the compound of Preparation 168 (117 mg, 0.305 mmol) was reacted with the compound from Preparation 41 (128 mg, 0.385 mmol) to afford the title compound as an off-white solid (90 mg, 43% yield). 1H NMR (400 MHz, DMSO-d6) δ 10.97 (s, 1H) 8.56 (d, J=8.61 Hz, 1H) 7.98-8.14 (m, 1H) 7.87 (dd, J=10.19, 8.23 Hz, 1H) 7.59 (d, J=1.96 Hz, 1H) 7.14 (d, J=2.07 Hz, 1H) 5.36 (s, 2H) 4.84-4.98 (m, 3H) 3.62 (t, J=7.14 Hz, 2H) 3.56 (t, J=7.90 Hz, 2H) 2.96 (s, 3H) 2.19 (s, 3H) 1.99 (s, 3H) 1.17 (t, J=7.14 Hz, 2H) 0.76-0.92 (m, 3H) 0.05-0.56 (m, 8H) −0.04 (s, 9H); LCMS (METHOD 2) (ESI): m/z: 688.4 [M+H]⁺; 95%; RT=2.69 min (ACQUITY BEH C18 column, 0.05% FA in water with MeCN).

### Preparation 170: methyl (2S)-3,3-dicyclopropyl-2-[[2-(3-methylsulfanylpropyl)pyrazole-3-carbonyl]amino]propanoate

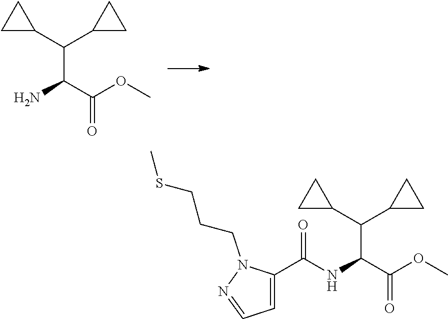

According to the method of Preparation 11 the compound of Preparation 161 (300 mg, 1.63 mmol) was reacted with the compound from Preparation 154 (360 mg, 1.80 mmol) to give the crude title compound as a yellow oil (600 mg, crude yield) which was used without characterisation.

### Preparation 171: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(3-methylsulfanylpropyl)pyrazole-3-carboxamide

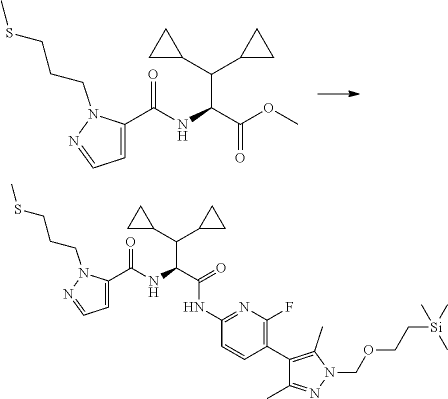

According to the method of Preparation 27 the crude compound of Preparation 170 (100 mg, 0.27 mmol) was reacted with the compound from Preparation 41 (100 mg, 0.3 mmol) to afford the title compound as an off-white solid (80 mg, 43% yield). 1H NMR (400 MHz, CDCl₃) δ 8.36 (s, 1H), 8.15 (dd, J=1.4, 8.1 Hz, 1H), 7.69 (dd, J=8.1, 9.4 Hz, 1H), 7.53 (d, J=2.1 Hz, 1H), 7.05-6.98 (m, 1H), 6.63 (d, J=2.1 Hz, 1H), 5.40 (s, 2H), 4.86 (dd, J=4.9, 7.9 Hz, 1H), 4.69 (qd, J=6.5, 12.8 Hz, 2H), 3.63 (br dd, J=7.7, 8.7 Hz, 2H), 2.54-2.46 (m, 2H), 2.26 (s, 3H), 2.19 (s, 3H), 2.17-2.15 (m, 2H) 2.09 (s, 3H), 0.96-0.77 (m, 5H), 0.72-0.54 (m, 4H), 0.40 (d, J=5.0 Hz, 2H), 0.31-0.25 (m, 2H), 0.02-0.00 (m, 9H); LCMS (METHOD 2) (ESI): m/z: 670 [M+H]⁺; 84%; RT=2.57 min (ACQUITY BEH C18 column, 0.1% FA in water with MeCN). Chiral analysis shows approx. 4:1 ratio of enantiomers.

### Preparation 172: methyl (2S)-3,3-dicyclopropyl-2-[[2-(3-methylsulfinylpropyl)pyrazole-3-carbonyl]amino]propanoate

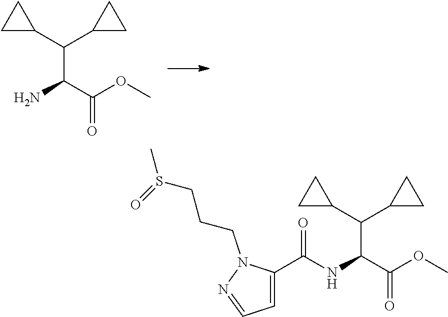 [SMILES: COC(=O)[C@@H](N)C(C1CC1)C1CC1 ; COC(=O)[C@@H](NC(=O)c1ccnn1CCCS(C)=O)C(C1CC1)C1CC1]

According to the method of Preparation 11 the compound of Preparation 161 (100 mg, 0.54 mmol) was reacted with the compound from Preparation 156 (120 mg, 0.60 mmol) to give the title compound as an off-white solid (100 mg, 48% yield). 1H NMR (400 MHz, DMSO-d6) δ 8.62 (br d, J=8.2 Hz, 1H), 7.53 (d, J=2.0 Hz, 1H), 7.04 (d, J=1.4 Hz, 1H), 4.68 (dd, J=6.4, 8.3 Hz, 1H), 4.59-4.52 (m, 2H), 3.7 (s, 3H) 2.72-2.64 (m, 1H), 2.63-2.53 (m, 1H), 2.13-2.00 (m, 2H), 1.03-0.92 (m, 1H), 0.84-0.64 (m, 3H), 0.52-0.13 (m, 9H), 0.07-0.01 (m, 1H); LCMS (METHOD 2) (ESI): m/z: 382 [M+H]⁺; 95%; RT=1.79 min (ACQUITY BEH C18 column, 0.1% FA in water with MeCN).

### Preparation 173: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(3-methylsulfinylpropyl)pyrazole-3-carboxamide

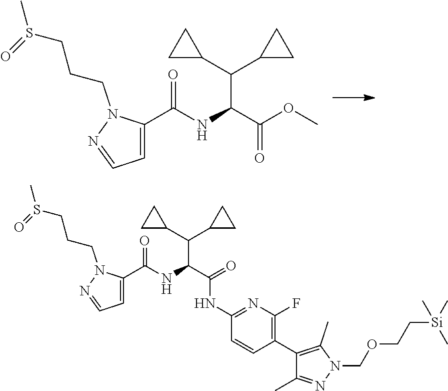 [SMILES: COC(=O)[C@@H](NC(=O)c1ccnn1CCCS(C)=O)C(C1CC1)C1CC1 ; Cc1nn(COCC[Si](C)(C)C)c(C)c1-c1ccc(NC(=O)[C@@H](NC(=O)c2ccnn2CCCS(C)=O)C(C2CC2)C2CC2)nc1F]

According to the method of Preparation 27 the compound of Preparation 172 (50 mg, 0.13 mmol) was reacted with the compound from Preparation 41 (49 mg, 0.15 mmol) to afford the title compound as an off-white solid (50 mg, 56% yield). LCMS (METHOD 2) (ESI): m/z: 686 [M+H]⁺; 66%; RT=2.25 min (ACQUITY BEH C18 column, 0.1% FA in water with MeCN).

### Preparation 174: methyl (2S)-3,3-dicyclopropyl-2-[[2-(3-methylsulfonylpropyl)pyrazole-3-carbonyl]amino]propanoate

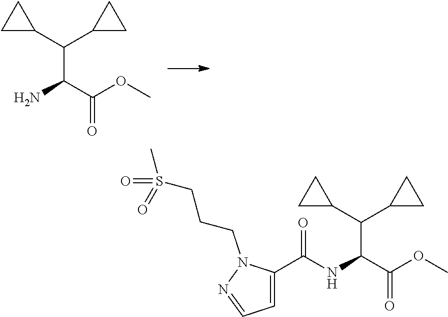 [SMILES: COC(=O)[C@@H](N)C(C1CC1)C1CC1 ; COC(=O)[C@@H](NC(=O)c1ccnn1CCCS(C)(=O)=O)C(C1CC1)C1CC1]

According to the method of Preparation 11 the compound of Preparation 161 (180 mg, 0.98 mmol) was reacted with the compound from Preparation 158 (251 mg, 1.08 mmol) to give the title compound as an off-white solid (120 mg, 31% yield). LCMS (METHOD 2) (ESI): m/z: 398 [M+H]⁺; 90%; RT=2.53 min (ACQUITY BEH C18 column, 0.1% FA in water with MeCN).

### Preparation 175: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(3-methylsulfonylpropyl)pyrazole-3-carboxamide

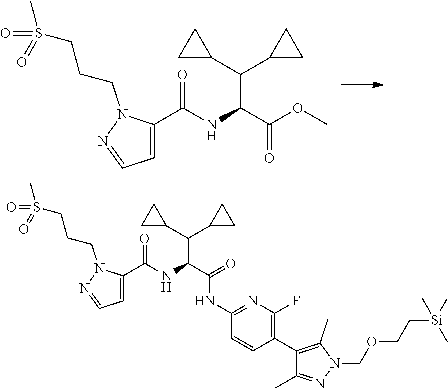 [SMILES: COC(=O)[C@@H](NC(=O)c1ccnn1CCCS(C)(=O)=O)C(C1CC1)C1CC1 ; Cc1nn(COCC[Si](C)(C)C)c(C)c1-c1ccc(NC(=O)[C@@H](NC(=O)c2ccnn2CCCS(C)(=O)=O)C(C2CC2)C2CC2)nc1F]

According to the method of Preparation 27 the compound of Preparation 174 (139 mg, 0.35 mmol) was reacted with the compound from Preparation 41 (129 mg, 0.38 mmol) to afford the title compound as an off-white solid (90 mg, 37% yield). LCMS (METHOD 2) (ESI): m/z: 702 [M+H]⁺; 76%; RT=2.68 min (ACQUITY BEH C18 column, 0.1% FA in water with MeCN).

### Preparation 176: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-propyl-pyrazole-3-carboxamide

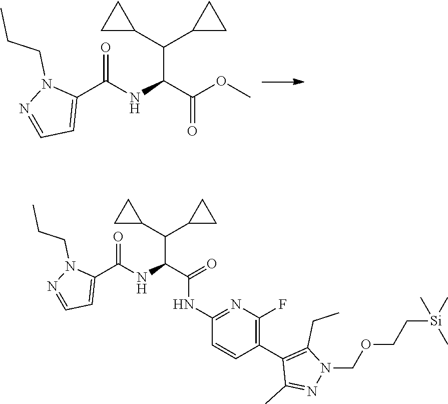 [SMILES: CCCn1nccc1C(=O)N[C@H](C(=O)OC)C(C1CC1)C1CC1 ; CCCn1nccc1C(=O)N[C@H](C(=O)Nc1ccc(-c2c(C)nn(COCC[Si](C)(C)C)c2CC)c(F)n1)C(C1CC1)C1CC1]

According to the method of Preparation 27 the compound of Preparation 162 (120 mg, 0.36 mmol) was reacted with the compound from Preparation 39 (145 mg, 0.41 mmol) to afford the title compound as an off-white solid (80 mg, 33% yield). 1H NMR (400 MHz, CDCl₃) δ 8.30 (s, 1H), 8.13 (dd, J=1.3, 8.1 Hz, 1H), 7.68 (dd, J=8.2, 9.4 Hz, 1H), 7.50 (d, J=2.1 Hz, 1H), 6.99 (d, J=7.7 Hz, 2H), 6.60 (d, J=2.1 Hz, 1H), 5.43-5.34 (m, 2H), 4.85 (dd, J=4.9, 7.9 Hz, 1H), 4.53 (dt, J=4.3, 7.3 Hz, 3H), 3.68-3.53 (m, 3H), 2.64 (d, J=7.6 Hz, 1H), 2.55 (d, J=7.6 Hz, 1H), 2.22 (s, 2H), 2.14 (s, 1H), 2.00-1.75 (m, 3H), 1.17-1.07 (m, 3H), 0.96-0.78 (m, 7H) 0.68-0.51 (m, 3H), 0.51-0.33 (m, 1H), 0.33-0.13 (m, 1H), 0.01 (s, 9H); LCMS (METHOD 2) (ESI): m/z: 638 [M+H]⁺; 85%; RT=2.62 min (ACQUITY BEH C18 column, 0.05% FA in water with MeCN).

### Preparation 177: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(2-methylsulfanylethyl)pyrazole-3-carboxamide

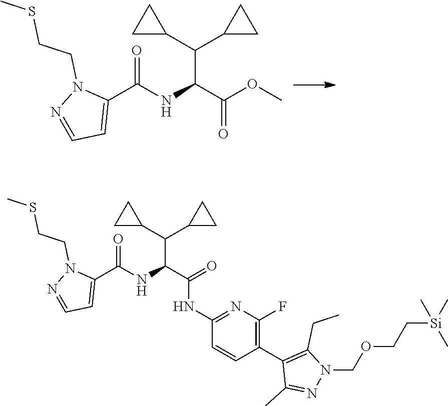

According to the method of Preparation 27 the compound of Preparation 164 (80 mg, 0.227 mmol) was reacted with the compound from Preparation 39 (88 mg, 0.25 mmol) to afford the title compound as an off-white solid (61 mg, 40% yield). LCMS (METHOD 2) (ESI): m/z: 670 [M+H]⁺; 90%; RT=2.89 min (ACQUITY BEH C18 column, 0.05% FA in water with MeCN).

### Preparation 178: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(2-methylsulfinylethyl)pyrazole-3-carboxamide

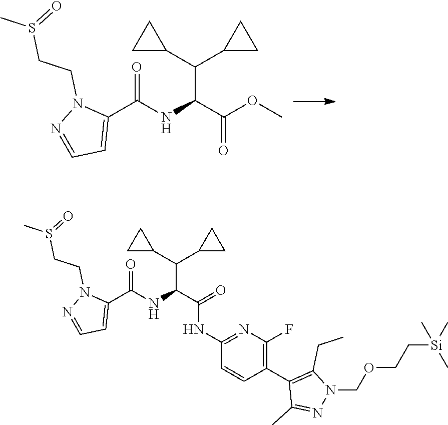

According to the method of Preparation 27 the compound of Preparation 166 (250 mg, 0.68 mmol) was reacted with the compound from Preparation 39 (262 mg, 0.75 mmol) to afford the title compound as an off-white solid (100 mg, 21% yield). 1H NMR (400 MHz, DMSO-d6) δ 10.96 (s, 1H), 8.59-8.50 (m, 1H), 8.06 (dd, J=1.5, 8.1 Hz, 1H), 7.90-7.80 (m, 1H), 7.57 (d, J=2.2 Hz, 1H), 7.10 (d, J=1.8 Hz, 1H), 5.41-5.33 (m, 2H), 4.97-4.72 (m, 3H), 3.64-3.50 (m, 2H), 3.29-3.02 (m, 2H), 2.68-2.51 (m, 6H), 2.17 (s, 2H), 1.11-0.73 (m, 8H), 0.58-0.07 (m, 8H), 0.03-0.11 (m, 9H); LCMS (METHOD 2) (ESI): m/z: 686 [M+H]⁺; 76%; RT=2.65 min (ACQUITY BEH C18 column, 0.1% FA in water with MeCN).

### Preparation 179: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(2-methylsulfonylethyl)pyrazole-3-carboxamide

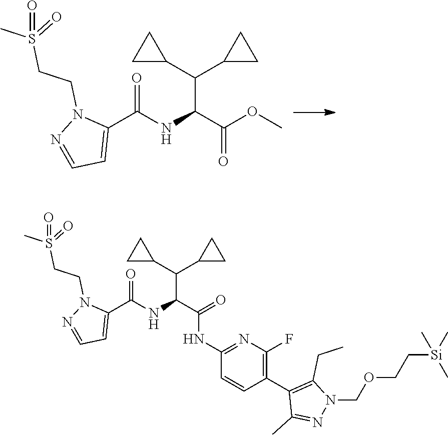

According to the method of Preparation 27 the compound of Preparation 168 (85 mg, 0.22 mmol) was reacted with the compound from Preparation 39 (85 mg, 0.24 mmol) to afford the title compound as an off-white solid (90 mg, 60% yield). 1H NMR (400 MHz, DMSO-d6) δ 10.97 (s, 1H) 8.56 (d, J=8.44 Hz, 1H) 7.98-8.12 (m, 1H) 7.85 (dd, J=10.27, 8.07 Hz, 1H) 7.59 (d, J=1.83 Hz, 1H) 7.13 (d, J=2.20 Hz, 1H) 5.37 (s, 2H) 4.80-4.93 (m, 3H) 3.46-3.74 (m, 4H) 2.96 (s, 3H) 2.51-2.75 (m, 7H) 1.04 (t, J=7.52 Hz, 2H) 0.90-0.96 (m, 1H) 0.72-0.87 (m, 4H) 0.08-0.54 (m, 7H) −0.04 (s, 9H); LCMS (METHOD 2) (ESI): m/z: 702 [M+H]⁺; 93%; RT=2.78 min (ACQUITY BEH C18 column, 0.05% FA in water with MeCN).

### Preparation 180: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(3-methylsulfanylpropyl)pyrazole-3-carboxamide

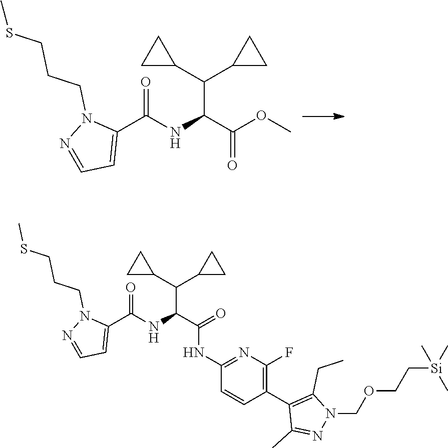

According to the method of Preparation 27 the crude compound of Preparation 170 (100 mg, 0.27 mmol) was reacted with the compound from Preparation 39 (105 mg, 0.3 mmol) to afford the title compound as an off-white solid (50 mg, 27% yield). LCMS (METHOD 2) (ESI): m/z: 684 [M+H]⁺; 80%; RT=3.00 min (ACQUITY BEH C18 column, 0.1% FA in water with MeCN).

### Preparation 181: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(3-methylsulfinylpropyl)pyrazole-3-carboxamide

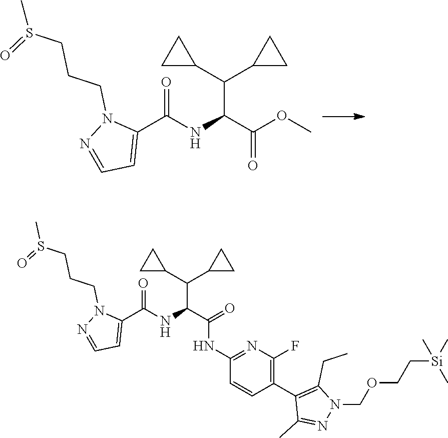 [SMILES: COC(=O)[C@@H](NC(=O)c1ccnn1CCCS(C)=O)C(C1CC1)C1CC1 ; CCc1c(-c2ccc(NC(=O)[C@@H](NC(=O)c3ccnn3CCCS(C)=O)C(C3CC3)C3CC3)nc2F)c(C)nn1COCC[Si](C)(C)C]

According to the method of Preparation 27 the compound of Preparation 172 (120 mg, 0.31 mmol) was reacted with the compound from Preparation 39 (121 mg, 0.35 mmol) to afford the title compound as an off-white solid (70 mg, 32% yield). 1H NMR (400 MHz, CDCl₃) δ 8.72-8.65 (m, 1H), 8.13 (dd, J=1.4, 8.0 Hz, 1H), 7.71-7.63 (m, 1H), 7.55-7.52 (m, 1H), 7.13-7.00 (m, 1H), 6.62 (t, J=1.9 Hz, 1H), 5.40 (s, 2H), 4.86-4.68 (m, 3H), 3.64-3.60 (m, 2H), 3.51-3.49 (m, 2H), 2.72-2.56 (m, 5H), 2.27-2.03 (m, 6H), 1.29 (dt, J=2.3, 4.5 Hz, 3H), 1.15-1.13 (m, 2H), 0.89-0.86 (m, 2H), 0.59-0.33 (m, 8H), -0.02 (s, 9H); LCMS (METHOD 2) (ESI): m/z: 700.5 [M+H]⁺; 71%; RT=2.67 min (ACQUITY BEH C18 column, 0.05% FA in water with MeCN).

### Preparation 182: N-[(1S)-1-(dicyclopropylmethyl)-2-[[5-[5-ethyl-3-methyl-1-(2-trimethylsilylethoxymethyl)pyrazol-4-yl]-6-fluoro-2-pyridyl]amino]-2-oxo-ethyl]-2-(3-methylsulfonylpropyl)pyrazole-3-carboxamide

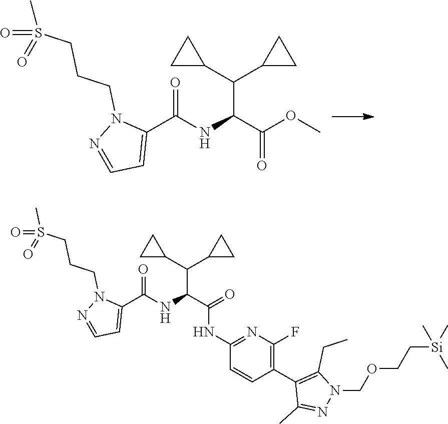 [SMILES: COC(=O)[C@@H](NC(=O)c1ccnn1CCCS(C)(=O)=O)C(C1CC1)C1CC1 ; CCc1c(-c2ccc(NC(=O)[C@@H](NC(=O)c3ccnn3CCCS(C)(=O)=O)C(C3CC3)C3CC3)nc2F)c(C)nn1COCC[Si](C)(C)C]

According to the method of Preparation 27 the compound of Preparation 174 (180 mg, 0.45 mmol) was reacted with the compound from Preparation 39 (175 mg, 0.50 mmol) to afford the title compound as an off-white solid (120 mg, 37% yield). LCMS (METHOD 2) (ESI): m/z: 716 [M+H]⁺; 98%; RT=2.13 min (ACQUITY BEH C18 column, 0.05% FA in water with MeCN).

### Preparation 183: (2S)-2-amino-3,3-dicyclopropyl-N-[5-(3,5-dimethyl-1H-pyrazol-4-yl)-6-fluoro-2-pyridyl]propenamide hydrochloride

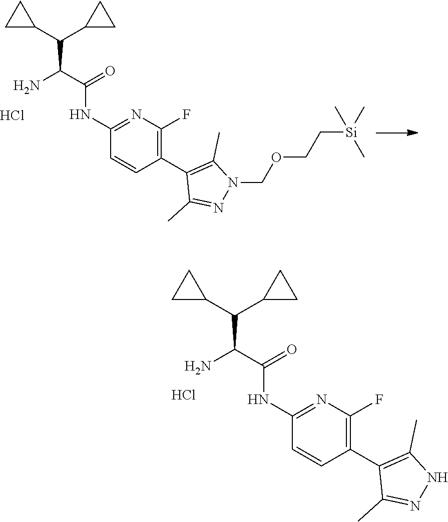 [SMILES: Cl ; Cc1nn(COCC[Si](C)(C)C)c(C)c1-c1ccc(NC(=O)[C@@H](N)C(C2CC2)C2CC2)nc1F ; Cc1n[nH]c(C)c1-c1ccc(NC(=O)[C@@H](N)C(C2CC2)C2CC2)nc1F ; Cl]

On prolonged storage of the compound of Preparation 92, loss of the SEM protecting group was observed. The resulting pyrazole was used without further purification. LCMS (METHOD 3) (ES): m/z 358.2 [M−H]⁻, RT=0.46 min.

### Preparation 184: methyl 6-benzyloxy-3-oxo-hexanoate

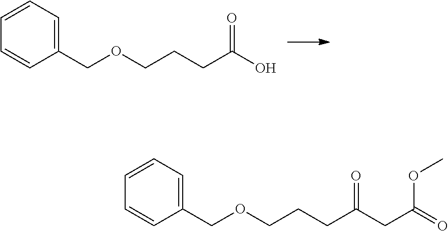 [SMILES: O=C(O)CCCOCc1ccccc1 ; COC(=O)CC(=O)CCCOCc1ccccc1]

CDI (10.0 g, 61.8 mmol) was added to a solution of 4-benzyloxybutanoic acid (10.0 g, 51.5 mmol) in dry THF (150 mL) at room temperature. The reaction mixture was stirred for 2 hours. Potassium 3-methoxy-3-oxo-propanoate (12.0 g, 77.2 mmol) and magnesium chloride (5.88 g, 61.8 mmol) were added and the resulting white suspension was stirred at room temperature for 18 hours. The pH was adjusted to −3 with hydrogen chloride (2M aqueous solution) and the mixture was extracted with Et₂O (2×100 mL). The combined extracts were dried over Na₂SO₄, filtered, and the obtained crude compound was purified by silica column chromatography (230-400 mesh), eluting with EtOAc in heptane, to afford the title compound as a colourless oil (10.1 g, 78% yield). 1H NMR (400 MHz, DMSO-d6) δ 7.46-7.20 (m, 5H), 4.43 (s, 2H), 3.61 (m, 5H), 3.41 (q, J=6.6 Hz, 2H), 2.60 (t, J=7.2 Hz, 2H), 1.84-1.66 (m, 2H).

### Preparation 185: methyl-6-benzyloxy-2-hydroxyimino-3-oxo-hexanoate

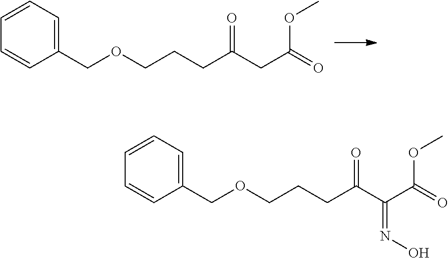 [SMILES: COC(=O)CC(=O)CCCOCc1ccccc1 ; COC(=O)/C(=N\O)C(=O)CCCOCc1ccccc1]

A solution of sodium nitrite (4.17 g, 60.4 mmol) in H₂O (20 mL) was added slowly to a solution of the compound of Preparation 184 (10.1 g, 40.3 mmol) in AcOH (35 mL) and H₂O (5 mL) at 5° C. The reaction mixture was stirred at between 5-10° C. for 4 hours. The reaction mixture was diluted with H₂O (200 mL) and extracted with Et₂O (2×100 mL). The combined organic layers were washed with saturated aq. NaHCO₃, dried over Na₂SO₄ and concentrated in vacuo to afford the crude title compound as a yellow oil. (11.1 g, assume 100% yield); LCMS (METHOD 3) (ES): m/z 278.2 [M−H]⁻, RT=0.68 min.

### Preparation 186: methyl-6-benzyloxy-2,3-bis(hydroxyimino)hexanoate

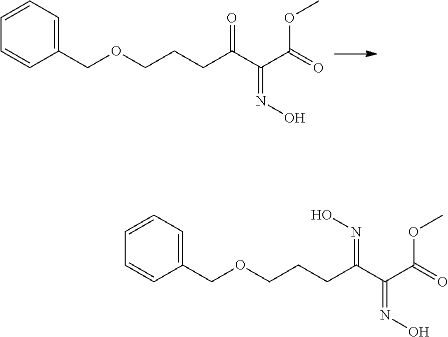 [SMILES: COC(=O)/C(=N\O)C(=O)CCCOCc1ccccc1 ; COC(=O)C(=N\O)/C(CCCOCc1ccccc1)=N/O]

Hydroxylamine hydrochloride (3.05 g, 43.8 mmol) was added to a solution of the compound of Preparation 185 (11.1 g, 39.9 mmol) and NaOAc (10.8 g, 79.7 mmol) in MeOH (20 mL). The reaction mixture was stirred at 50° C. for 18 hours. The reaction mixture was diluted with brine solution (50 mL) and extracted with EtOAc (2×100 mL). The combined organic extracts were dried over Na₂SO₄, filtered, and the obtained crude compound was purified by silica column chromatography (230-400 mesh), eluting with EtOAc in heptane, to afford the title compound as a colourless oil (6.4 g, 55% yield); LCMS (METHOD 3) (ES): m/z 293.2 [M−H]⁻, RT=0.62 min.

### Preparation 187: methyl 4-(3-benzyloxypropyl)-1,2,5-oxadiazole-3-carboxylate

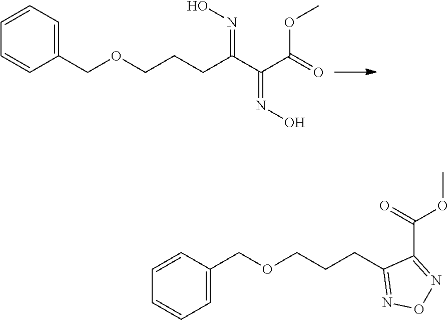 [SMILES: COC(=O)C(=N\O)/C(CCCOCc1ccccc1)=N/O ; COC(=O)c1nonc1CCCOCc1ccccc1]

CDI (780 mg, 4.80 mmol) was added to a solution of the compound of Preparation 186 (940 mg, 3.20 mmol) in MeCN (20 mL) and stirred at room temperature for 2 days. The reaction mixture was diluted with citric acid (3% solution, 10 mL) and extracted with Et₂O (2×25 mL). The combined organic extracts were dried over Na₂SO₄, filtered, and the obtained crude compound was purified by silica column chromatography (230-400 mesh), eluting with EtOAc in heptane, to afford the title compound as a colourless oil (294 mg, 33% yield). 1H NMR (400 MHz, CDCl₃) δ 7.40-7.24 (m, 5H), 4.49 (s, 2H), 4.00 (s, 3H), 3.57 (t, J=6.0 Hz, 2H), 3.12 (dd, J=8.0, 7.0 Hz, 2H), 2.15-2.04 (m, 2H).

### Preparation 188: 4-(3-benzyloxypropyl)-1,2,5-oxadiazole-3-carboxylic acid

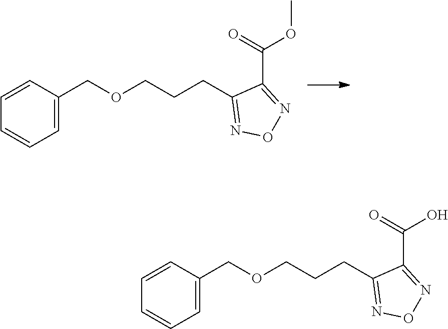 [SMILES: COC(=O)c1nonc1CCCOCc1ccccc1 ; O=C(O)c1nonc1CCCOCc1ccccc1]

LiOH·H₂O (74.0 mg, 1.76 mmol) in H₂O (4 mL) was added to a solution of the compound of Preparation 187 (300 mg, 1.10 mmol) in THF (5 mL) and stirred at room temperature for 30 minutes. The pH of the reaction mixture was adjusted to −3 with hydrogen chloride (2M aqueous solution). The mixture was extracted with Et₂O (2×20 mL). The combined extracts were dried over Na₂SO₄, filtered and concentrated in vacuo to afford the title compound as a colourless oil (280 mg, 98% yield).

### Preparation 189: 4-(3-benzyloxypropyl)-N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-1,2,5-oxadiazole-3-carboxamide

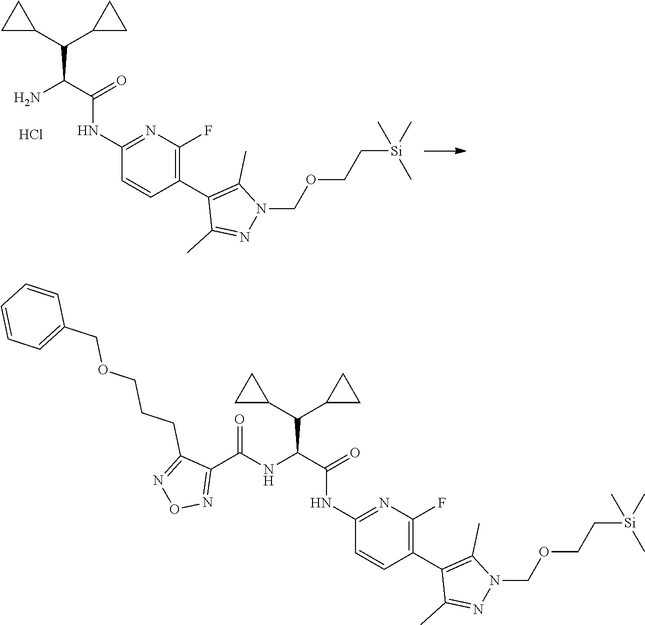 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2nonc2CCCOCc2ccccc2)C(C2CC2)C2CC2)nc1F]

According to the method of Preparation 11 the compound of Preparation 92 (78 mg, 0.16 mmol) was reacted with the compound from Preparation 188 (62.9 mg, 0.24 mmol) to afford the title compound that was used directly without purification (assume 100% yield). LCMS (METHOD 3) (ES): m/z 732.5 [M+H]⁺, RT=1.08 min.

### Preparation 190: 4-(2-benzyloxypropyl)-N-[(1S)-1-(dicyclopropylmethyl)-2-[[5-(3,5-dimethyl-1H-pyrazol-4-yl)-6-fluoro-2-pyridyl]amino]-2-oxo-ethyl]-1,2,5-oxadiazole-3-carboxamide

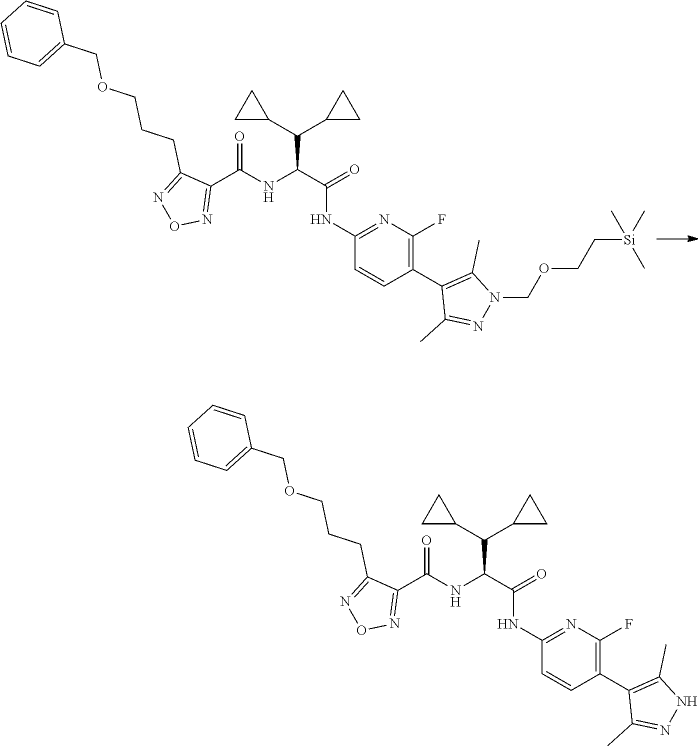 [SMILES: Cc1nn(COCC[Si](C)(C)C)c(C)c1-c1ccc(NC(=O)[C@@H](NC(=O)c2nonc2CCCOCc2ccccc2)C(C2CC2)C2CC2)nc1F ; Cc1n[nH]c(C)c1-c1ccc(NC(=O)[C@@H](NC(=O)c2nonc2CCCOCc2ccccc2)C(C2CC2)C2CC2)nc1F]

According to the method of Example 1 the compound of Preparation 189 (115 mg, 0.16 mmol) was reacted to afford the title compound after prep. basic HPLC (66 mg, 69% yield). LCMS (METHOD 3) (ES): m/z 602.4 [M+H]⁺, RT=0.90 min.

### Preparation 191: ethyl (2Z)-3-cyclopropyl-2-hydroxyimino-3-oxo-propanoate

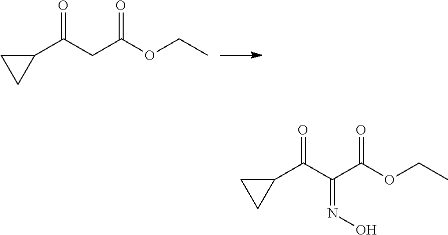

According to the method of Preparation 185 ethyl 3-cyclopropyl-3-oxo-propanoate (200 g, 1.28 mol) was reacted to afford the title compound that was used directly without purification (160 g, 67% yield). 1H NMR (300 MHz, CDCl₃) δ 9.81 (ds, 1H), 4.39 (q, J=7.0 Hz, 2H), 2.75-2.68 (m, 1H), 1.35 (d, J=7.2 Hz, 3H), 1.99-1.15 (m, 2H), 1.074-1.037 (m, 2H); LCMS (METHOD 2) (ESI): m/z: 186 [M−H]⁻; 94%; RT=1.6 min (ACQUITY BEH C18 column, mobile phase; A: 0.05% FA in water with MeCN).

### Preparation 192: ethyl (2Z,3E)-3-cyclopropyl-2,3-bis(hydroxyimino)propanoate

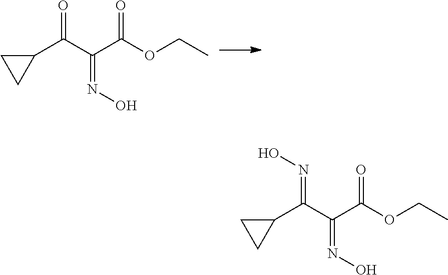

According to the method of Preparation 186 the compound of Preparation 191 (100 g, 0.54 mol) was reacted to afford the title compound that was used directly without purification (50 g, 46% yield). LCMS (METHOD 2) (ESI): m/z: 201 [M+H]⁺; 82%; RT=1.40 min (ACQUITY BEH C18 column, mobile phase; A: 0.05% FA in water with MeCN).

### Preparation 193: ethyl 4-cyclopropyl-1,2,5-oxadiazole-3-carboxylate

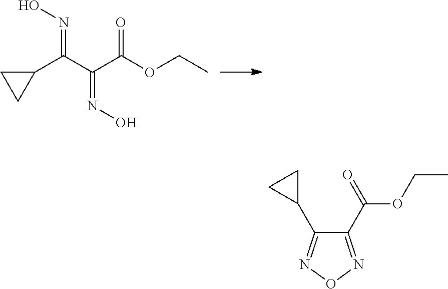

CDI (48.6 g, 300 mmol) was added to a solution of the compound of Preparation 192 (40 g, 200 mmol) in THF (600 mL) at room temperature and stirred for 16 hours. The reaction mixture was concentrated in vacuo and the obtained crude material was purified by column chromatography (EtOAc in hexane) to afford the title compound as a colourless oil (2.5 g, 6.8% yield). 1H NMR (300 MHz, CDCl₃) δ 4.51 (q, J=7.2 Hz, 2H), 2.45-2.41 (m, 1H), 1.47 (d, J=7.2 Hz, 3H), 1.21-1.167 (m, 4H).

### Preparation 194: 4-cyclopropyl-1,2,5-oxadiazole-3-carboxylate; lithium salt

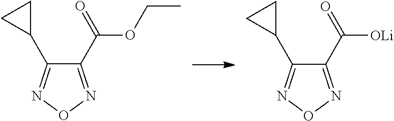

LiOH·H₂O (2M soln, 2.5 mL) was added to a solution of the compound of Preparation 193 (250 mg, 1.37 mmol) in THF:H₂O (5 mL, 1:1) and stirred at room temperature for 4 hours. The reaction mixture was and concentrated in vacuo and distilled with toluene (2×10 mL), to afford the title compound as an off-white solid (200 mg, 93% yield). 1H NMR (400 MHz, DMSO-d6) δ 2.47-2.43 (m, 1H), 1.05-1.01 (m, 2H), 0.92-0.90 (m, 2H); LCMS (METHOD 2) (ESI): m/z: 153 [M+H]⁺; 96%; RT=1.39 min (ACQUITY BEH C18 column, mobile phase; A: 0.05% FA in water with MeCN);

### Preparation 195: 4-cyclopropyl-N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-1,2,5-oxadiazole-3-carboxamide

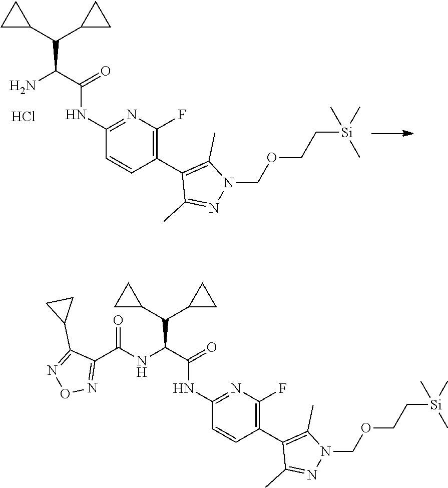 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2nonc2C2CC2)C(C2CC2)C2CC2)nc1F]

According to the method of Preparation 11 the compound of Preparation 92 (152 mg, 0.31 mmol) was reacted with the compound from Preparation 194 (50 mg, 0.31 mmol) to afford the crude title compound (50 mg, 26% yield). LCMS (METHOD 2) (ESI): m/z: 624 [M+H]⁺; 73%; RT=2.67 min (ACQUITY BEH C18 column, 0.05% FA in water with MeCN).

### Preparation 196: methyl 3-propyltriazole-4-carboxylate

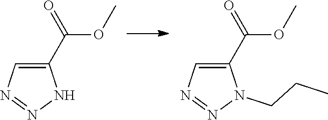

1-Iodopropane (1.69 mL, 17.3 mmol) was added to a mixture of methyl 1H-triazole-5-carboxylate (2.0 g, 15.7 mmol) and K₂CO₃ (1.3 g, 9.44 mmol) in DMF (25 mL) and stirred at room temperature for 16 hours. The reaction mixture was filtered and concentrated in vacuo. The crude material was diluted with H₂O (15 ml) and extracted with DCM (3×25 ml). The combined organic layers were dried over Na₂SO₄, filtered and concentrated in vacuo. the obtained crude compound was purified by silica column chromatography (230-400 mesh), eluting with EtOAc in heptane, to afford the title compound as a colourless solid (200 mg, 8% yield). 1H NMR (400 MHz, DMSO-d6) δ 8.30 (s, 1H), 4.63 (t, J=7.1 Hz, 2H), 3.88 (s, 3H), 1.88-1.76 (m, 2H), 0.85 (t, J=7.4 Hz, 3H); LCMS (METHOD 2) (ESI): m/z: 169.9 [M+H]⁺; 87%; RT=1.5 min (ACQUITY BEH C18 column, 0.1% FA in water with MeCN).

### Preparation 197: 3-propyltriazole-4-carboxylic acid

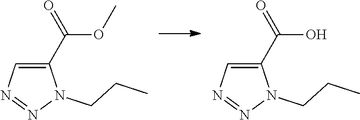

According to the method of Preparation 148 the compound of Preparation 196 (200 mg, 1.18 mmol) was reacted to afford the crude title compound (160 mg, 87% yield). 1H NMR (300 MHz, DMSO-d6) δ 14.1 (ds, 1H), 8.22 (s, 1H), 4.63 (t, J=7.2 Hz, 2H), 1.89-1.73 (m, 2H), 0.84 (t, J=7.3 Hz, 3H); LCMS (METHOD 2) (ESI): m/z: 156 [M+H]⁺; 98%; RT=1.32 min (ACQUITY BEH C18 column, 0.05% FA in water with MeCN).

### Preparation 198: methyl (2S)-3,3-dicyclopropyl-2-[(3-propyltriazole-4-carbonyl)amino]propanoate

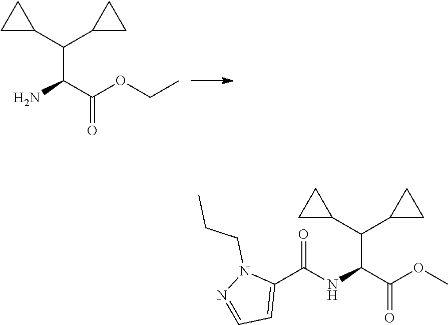 [SMILES: CCOC(=O)[C@@H](N)C(C1CC1)C1CC1 ; CCCn1nccc1C(=O)N[C@H](C(=O)OC)C(C1CC1)C1CC1]

According to the method of Preparation 11 the compound of Preparation 37 (120 mg, 0.61 mmol) was reacted with the compound of Preparation 197 (104 mg, 0.67 mmol) to give the title compound as an off-white solid (90 mg, 44% yield). 1H NMR (400 MHz, DMSO-d6) δ 8.95 (d, J=8.4 Hz, 1H), 8.33 (s, 1H), 4.69 (dd, J=6.1, 8.5 Hz, 1H), 4.60 (q, J=6.8 Hz, 2H), 4.12 (t, J=7.1 Hz, 2H), 1.82-1.74 (m, 2H), 1.27-1.21 (m, 3H), 1.03-0.95 (m, 1H), 0.84-0.78 (m, 4H), 0.69-0.64 (m, 1H), 0.50-0.41 (m, 2H), 0.33-0.13 (m, 6H); LCMS (METHOD 2) (ESI): m/z: 335 [M+H]⁺; 91%; RT=2.48 min (ACQUITY BEH C18 column, 0.05% FA in water with MeCN).

### Preparation 199: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-3-propyl-triazole-4-carboxamide

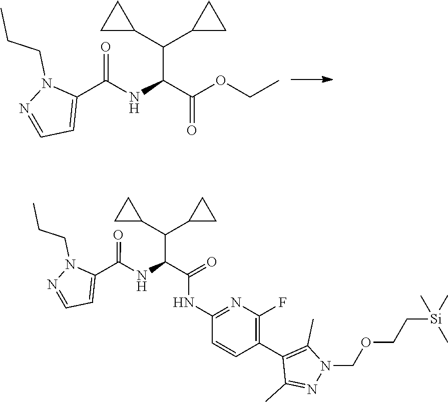

According to the method of Preparation 27 the compound of Preparation 198 (90 mg, 0.27 mmol) was reacted with the compound from Preparation 41 (81 mg, 0.24 mmol) to afford the title compound as an off-white solid (35 mg, 20% yield). LCMS (METHOD 2) (ESI): m/z: 625 [M+H]⁺; 90%; RT=2.88 min (ACQUITY BEH C18 column, 0.05% FA in water with MeCN).

### Preparation 200: methyl 3-sec-butyltriazole-4-carboxylate

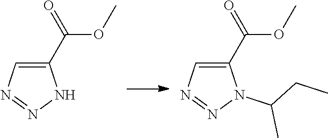

According to the method of Preparation 196, methyl 1H-triazole-5-carboxylate (700 mg, 4.96 mmol) was reacted with 2-bromobutane (928 mg, 5.46 mmol) to give the title compound as an off-white solid (200 mg, 22% yield). 1H NMR (400 MHz, DMSO-d6) δ 8.29 (s, 1H), 5.25 (td, J=6.5, 7.8 Hz, 1H), 3.88 (s, 3H), 2.03-1.85 (m, 2H), 1.53 (d, J=6.8 Hz, 3H), 0.74 (t, J=7.4 Hz, 3H).

### Preparation 201: 3-sec-butyltriazole-4-carboxylic acid

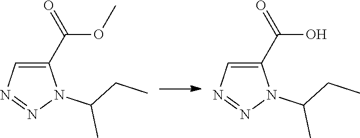

According to the method of Preparation 148, the compound of Preparation 200 (300 mg, 1.63 mmol) was reacted to afford the crude title compound (2000 mg, 72% yield). 1H NMR (300 MHz, DMSO-d6) δ 14.19-13.73 (m, 1H), 8.19 (s, 1H), 5.38-5.22 (m, 1H), 2.07-1.81 (m, 2H), 1.52 (d, J=6.6 Hz, 3H), 0.73 (t, J=7.3 Hz, 3H); LCMS (METHOD 2) (ESI): m/z: 170 [M+H]⁺; 99%; RT=1.58 min (ACQUITY BEH C18 column, 0.05% FA in water with MeCN).

### Preparation 202: methyl (2S)-3,3-dicyclopropyl-2-[(3-sec-butyltriazole-4-carbonyl)amino]propanoate

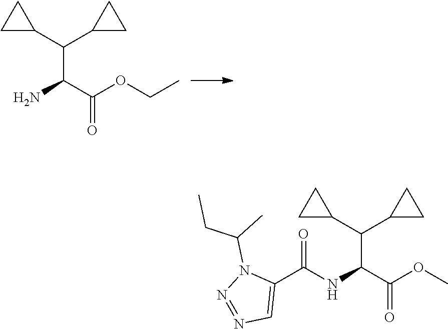 [SMILES: CCOC(=O)[C@@H](N)C(C1CC1)C1CC1 ; CCC(C)n1nncc1C(=O)N[C@H](C(=O)OC)C(C1CC1)C1CC1]

According to the method of Preparation 11 the compound of Preparation 37 (120 mg, 0.61 mmol) was reacted with the compound of Preparation 201 (113 mg, 0.67 mmol) to give the title compound as an off-white solid (150 mg, 74% yield). 1H NMR (400 MHz, DMSO-d6) δ 8.97 (dd, J=14.28, 8.50 Hz, 1H) 8.26 (d, J=8.61 Hz, 1H) 5.06-5.27 (m, 1H) 4.68 (td, J=8.12, 6.21 Hz, 1H) 4.0-4.26 (m, 2H) 1.70-2.00 (m, 2H) 1.50 (dd, J=8.61, 6.76 Hz, 2H) 1.22 (td, J=7.14, 3.27 Hz, 4H) 0.98-1.0 (m, 1H) 0.59-0.88 (m, 5H) 0.05-0.53 (m, 8H); LCMS (METHOD 2) (ESI): m/z: 349 [M+H]⁺; 85%; RT=2.56 min (ACQUITY BEH C18 column, 0.05% FA in water with MeCN).

### Preparation 203: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-3-sec-butyl-triazole-4-carboxamide

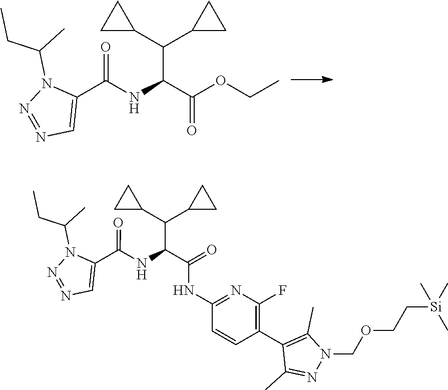

According to the method of Preparation 27 the compound of Preparation 202 (120 mg, 0.36 mmol) was reacted with the compound from Preparation 41 (108 mg, 0.32 mmol) to afford the title compound as an off-white solid (90 mg, 39% yield). LCMS (METHOD 2) (ESI): m/z: 639 [M+H]⁺; 94%; RT=2.93 min (ACQUITY BEH C18 column, 0.05% FA in water with MeCN); Chiral HPLC: 46% (RT: 4.35 min) & 48% (RT: 5.0 min) Column: CHIRALPAK IE-3 (4.6*150 mm) 3 μm, Co-Solvent: 0.5% DEA in Methanol (40%), Column Temperature:

30° C., Flow: 3 g/min, ABPR: 1500 psi.

### Preparation 204: methyl 3-[2-fluoro-1-(fluoromethyl)ethyl]triazole-4-carboxylate

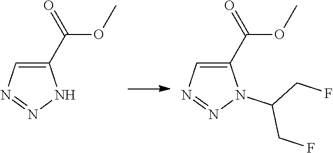

According to the method of Preparation 147, methyl 1H-triazole-5-carboxylate (2.0 g, 15.7 mmol) was reacted with 1,3-difluoropropan-2-ol (1.50 g, 15.7 mmol). The obtained crude compound was purified by silica column chromatography (230-400 mesh), eluting with EtOAc in heptane, to afford the title compound as a colourless oil (650 mg, 40% yield). 1H NMR (300 MHz, CDCl₃) δ 8.19 (s, 1H), 5.96 (tt, J=5.9, 16.0 Hz, 1H), 5.12-5.00 (m, 2H), 4.96-4.85 (m, 2H), 3.96 (s, 3H); LCMS (METHOD 2) (ESI): m/z: 206 [M+H]⁺; 98%; RT=1.82 min (ACQUITY BEH C18 column, 0.05% FA in water with MeCN).

### Preparation 205: 3-[2-fluoro-1-(fluoromethyl)ethyl]triazole-4-carboxylic acid

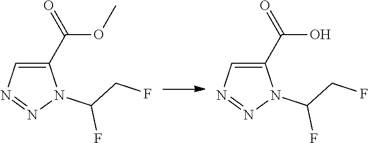

Hydrogen chloride (6M aq. soln, 4.0 mL) was added to a solution of the compound of Preparation 204 (400 mg, 1.95 mmol) in 1,4-dioxane (4 mL). The reaction mixture was stirred at 100° C. for 24 hours, then cooled and concentrated in vacuo. The solid was triturated with Et₂O to afford the title compound as an off-white solid (320 mg, 86% yield). 1H NMR (400 MHz, DMSO-d6) δ 14.1 (ds, 1H), 8.33 (s, 1H), 5.94-5.96 (m, 1H), 5.12-4.99 (m, 2H), 4.97-4.92 (m, 2H); LCMS (METHOD 2) (ESI): m/z: 192 [M+H]⁺; 95%; RT=0.71 min (ACQUITY BEH C18 column, 0.05% FA in water with MeCN).

### Preparation 206: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-3-[2-fluoro-1-(fluoromethyl)ethyl]triazole-4-carboxamide

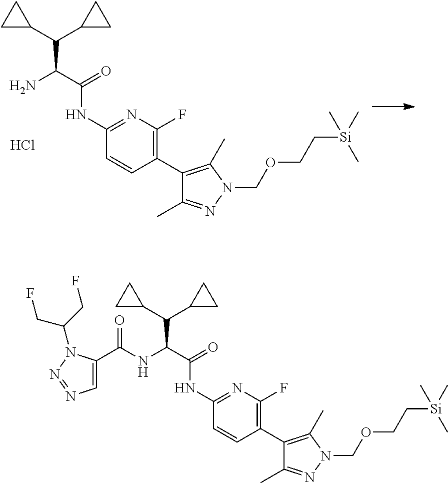 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2cnnn2C(CF)CF)C(C2CC2)C2CC2)nc1F]

According to the method of Preparation 11 the compound of Preparation 92 (100 mg, 0.20 mmol) was reacted with the compound from Preparation 205 (42.5 mg, 0.22 mmol) to afford the title compound after trituration with Et₂O (100 mg, 75% yield). LCMS (METHOD 2) (ESI): m/z: 661 [M+H]⁺; 68%; RT=2.87 min (ACQUITY BEH C18 column, 0.05% FA in water with MeCN).

### Preparation 207: ethyl 1-methyltetrazole-5-carboxylate

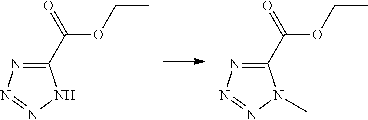

Iodomethane (0.88 mL, 14.1 mmol) was added to a turbid mixture of ethyl 1H-tetrazole-5-carboxylate (1.00 g, 7.03 mmol) and Cs₂CO₃ (2.29 g, 7.03 mmol) in DMF (15 mL) at room temperature. After 1 hour the now clear solution was diluted with Et₂O and H₂O (20 mL each). The aqueous phase was rewashed with Et₂O (20 mL) and the combined organic phases were washed with H₂O (20 mL), saturated brine solution (20 mL), dried over MgSO₄, filtered and concentrated in vacuo. The obtained mixture of regioisomers was purified by basic prep. HPLC to afford the title compound (104 mg, 9% yield). 1H NMR (400 MHz, CDCl₃) δ 4.53 (q, J=7.1 Hz, 2H), 4.46 (s, 3H), 1.47 (t, J=7.1 Hz, 3H); LCMS (METHOD 3) (ES): m/z 157.1 [M+H]⁺, RT=0.40 min.

### Preparation 208: cesium; 1-methyltetrazole-5-carboxylate

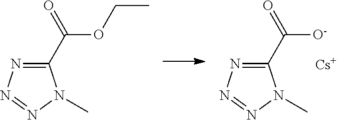

Cesium hydroxide (20.0 mg, 0.12 mmol) in H₂O (0.2 mL) was added to a solution of the compound of Preparation 207 (16.0 mg, 0.10 mmol) in MeOH (1.0 mL). The reaction mixture was stirred at room temperature for 1 hour then concentrated in vacuo to give crude title compound that was used directly in the next Preparation. (26.0 mg, assume 100% yield).

### Preparation 209: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-1-methyl-tetrazole-5-carboxamide

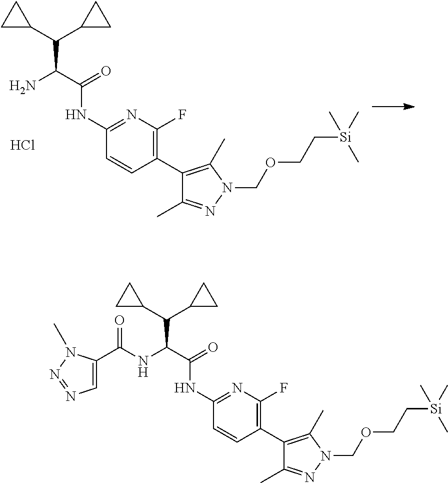

According to the method of Preparation 11 the compound of Preparation 92 (27 mg, 0.06 mmol) was reacted with the compound from Preparation 208 (21.6 mg, 0.08 mmol) to afford the title compound after acidic prep. HPLC (29 mg, 87% yield). LCMS (METHOD 3) (ES): m/z 598.3 [M+H]⁺, RT=0.94 min.

### Preparation 210: ethyl 2-[2-fluoro-1-(fluoromethyl)ethyl]pyrazole-3-carboxylate

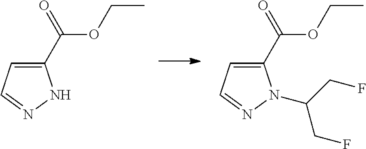

According to the method of Preparation 147, ethyl 1H-pyrazole-5-carboxylate (250 mg, 1.78 mmol) was reacted with 1,3-difluoropropan-2-ol (240 mg, 2.50 mmol). The obtained crude compound was purified by silica column chromatography (230-400 mesh), eluting with EtOAc in heptane, to afford the title compound as a colourless oil (282 mg, 72% yield). 1H NMR (400 MHz, CDCl₃) δ 7.59 (d, J=2.5 Hz, 1H), 6.90 (d, J=2.4 Hz, 1H), 5.96 (ddt, J=22.1, 11.3, 5.8 Hz, 1H), 5.04-4.74 (m, 4H), 4.43-4.27 (m, 2H), 1.45-1.31 (m, 3H); LCMS (METHOD 3) (ES): m/z 219.2 [M+H]⁺, RT=0.66 min.

### Preparation 211: 2-[2-fluoro-1-(fluoromethyl)ethyl]pyrazole-3-carboxylic acid

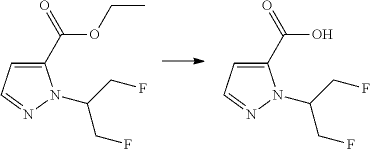

According to the method of Preparation 148, the compound of Preparation 210 (282 mg, 1.29 mmol) was reacted to afford the crude title compound (245 mg, assume 100% yield). LCMS (METHOD 3) (ES): m/z 191.1 [M+H]⁺, RT=0.36 min.

### Preparation 212: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-[2-fluoro-1-(fluoromethyl)ethyl]pyrazole-3-carboxamide

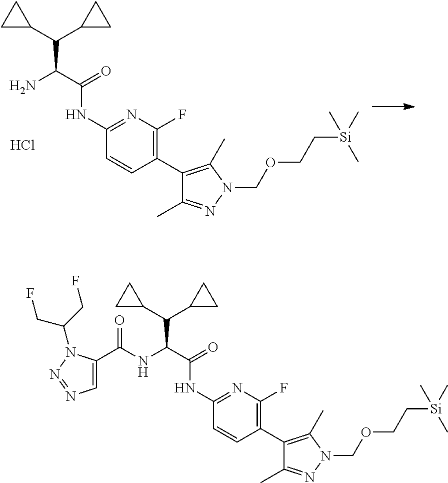 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2cnnn2C(CF)CF)C(C2CC2)C2CC2)nc1F]

According to the method of Preparation 11 the compound of Preparation 92 (50 mg, 0.102 mmol) was reacted with the compound from Preparation 211 (19.5 mg, 0.102 mmol) to afford the title compound after acidic prep. HPLC (38 mg, 56% yield). LCMS (METHOD 3) (ES): m/z 660.6 [M+H]⁺, RT=0.96 min.

### Preparation 213: ethyl 2-[(3,3-difluorocyclobutyl)methyl]pyrazole-3-carboxylate

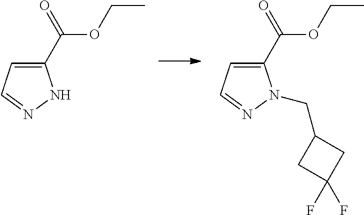

According to the method of Preparation 147, ethyl 1H-pyrazole-5-carboxylate (260 mg, 1.86 mmol) was reacted with (3,3-difluorocyclobutyl)methanol (317 mg, 2.60 mmol). The obtained crude compound was purified by silica column chromatography (230-400 mesh), eluting with EtOAc in heptane, to afford the title compound as a colourless oil (294 mg, 65% yield). 1H NMR (400 MHz, CDCl₃) δ 7.49 (d, J=2.0 Hz, 1H), 6.85 (d, J=2.0 Hz, 1H), 4.69 (d, J=7.1 Hz, 2H), 4.35 (q, J=7.1 Hz, 2H), 2.79-2.55 (m, 3H), 2.51-2.36 (m, 2H), 1.38 (t, J=7.1 Hz, 3H); LCMS (METHOD 3) (ES): m/z 245.2 [M+H]⁺, RT=0.76 min.

### Preparation 214: 2-[(3,3-difluorocyclobutyl)methyl]pyrazole-3-carboxylic acid

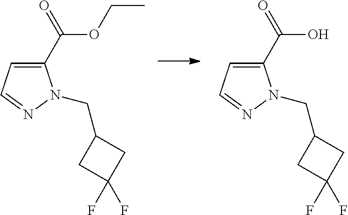 [SMILES: CCOC(=O)c1ccnn1CC1CC(F)(F)C1 ; O=C(O)c1ccnn1CC1CC(F)(F)C1]

According to the method of Preparation 148, the compound of Preparation 213 (295 mg, 1.35 mmol) was reacted to afford the crude title compound (253 mg, 97% yield). LCMS (METHOD 3) (ES): m/z 215.1 [M−H]⁻, RT=0.49 min.

### Preparation 215: ethyl 2-[(1-methylazetidin-3-yl)methyl]pyrazole-3-carboxylate

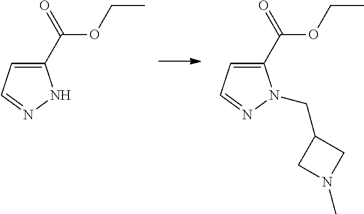

According to the method of Preparation 147, ethyl 1H-pyrazole-5-carboxylate (257 mg, 1.83 mmol) was reacted with (1-methylazetidin-3-yl)methanol (0.26 mL, 2.57 mmol). The obtained crude compound was purified by silica column chromatography (230-400 mesh), eluting with EtOAc in heptane, to afford the title compound as a colourless oil (172 mg, 42% yield). ¹H NMR (400 MHz, CDCl₃) δ 7.46 (d, J=2.0 Hz, 1H), 6.82 (d, J=2.0 Hz, 1H), 4.60 (d, J=7.3 Hz, 2H), 4.34 (q, J=7.1 Hz, 2H), 2.85 (h, J=7.7 Hz, 1H), 2.06-1.93 (m, 2H), 1.93-1.75 (m, 4H), 1.38 (t, J=7.1 Hz, 3H); LCMS (METHOD 3) (ES): m/z 224.2 [M+H]⁺, RT=0.40 min.

### Preparation 216: 2-[(1-methylazetidin-3-yl)methyl]pyrazole-3-carboxylic acid

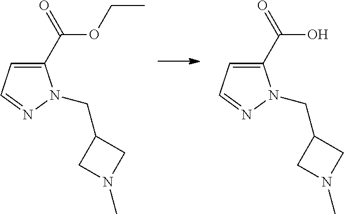 [SMILES: CCOC(=O)c1ccnn1CC1CN(C)C1 ; CN1CC(Cn2nccc2C(=O)O)C1]

According to the method of Preparation 148, the compound of Preparation 215 (172 mg, 0.77 mmol) was reacted to afford the crude title compound (150 mg, assume 100% yield). LCMS (METHOD 3) (ES): m/z 196.1 [M+H]⁺, RT=0.18 min.

### Preparation 217: ethyl 2-[(1-tert-butoxycarbonylazetidin-3-yl)methyl]pyrazole-3-carboxylate

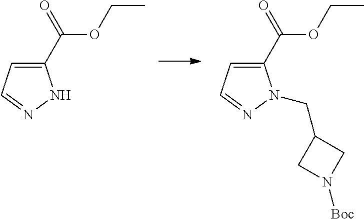

According to the method of Preparation 147, ethyl 1H-pyrazole-5-carboxylate (250 mg, 1.78 mmol) was reacted with tert-butyl 3-(hydroxymethyl)azetidine-1-carboxylate (467 mg, 2.50 mmol). The obtained crude compound was purified by silica column chromatography (230-400 mesh), eluting with EtOAc in heptane, to afford the title compound as a colourless oil (550 mg, assume 100% yield). LCMS (METHOD 3) (ES): m/z 310.2 [M+H]⁺, RT=0.78 min.

### Preparation 218: 2-[(1-tert-butoxycarbonylazetidin-3-yl)methyl]pyrazole-3-carboxylic acid

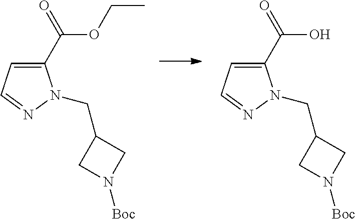 [SMILES: CCOC(=O)c1ccnn1CC1CN(C(=O)OC(C)(C)C)C1 ; CC(C)(C)OC(=O)N1CC(Cn2nccc2C(=O)O)C1]

According to the method of Preparation 148, the compound of Preparation 217 (550 mg, 1.78 mmol) was reacted to afford the crude title compound (361 mg, assume 61% yield). LCMS (METHOD 3) (ES): m/z 280.2 [M−H]⁻, RT=0.55 min.

### Preparation 219: tert-butyl 3-[[5-[[(1S)-1-(dicyclopropylmethyl)-2-[[5-(3,5-dimethyl-1H-pyrazol-4-yl)-6-fluoro-2-pyridyl]amino]-2-oxo-ethyl]carbamoyl]pyrazol-1-yl]methyl]azetidine-1-carboxylate

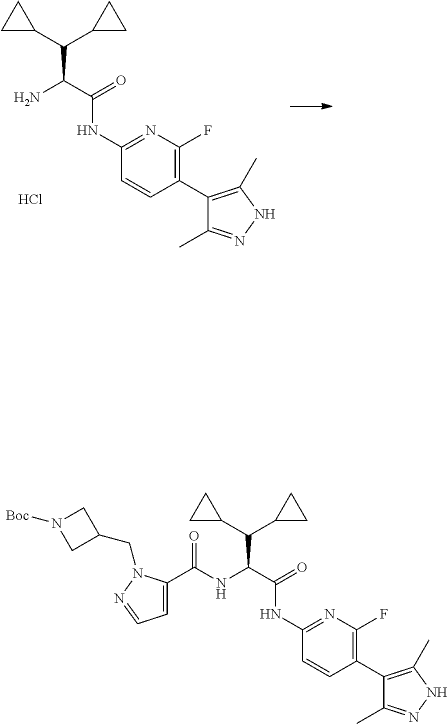 [SMILES: Cc1n[nH]c(C)c1-c1ccc(NC(=O)[C@@H](N)C(C2CC2)C2CC2)nc1F ; Cl ; Cc1n[nH]c(C)c1-c1ccc(NC(=O)[C@@H](NC(=O)c2ccnn2CC2CN(C(=O)OC(C)(C)C)C2)C(C2CC2)C2CC2)nc1F]

According to the method of Preparation 11 the compound of Preparation 92 (20 mg, 0.05 mmol) was reacted with the compound from Preparation 218 (17.1 mg, 0.06 mmol) to afford the crude title compound (31 mg, assume 100% yield).

### Preparation 220: Ethyl 2-[(1S)-2-benzyloxy-1-methyl-ethyl]pyrazole-3-carboxylate

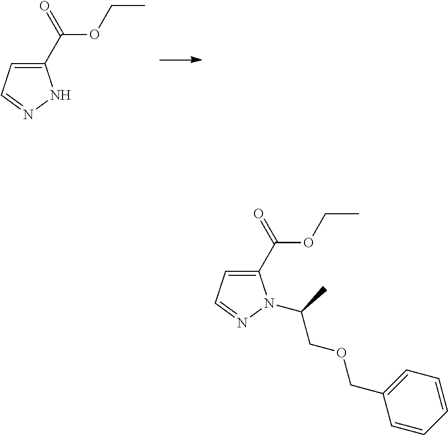

Diethyl azodicarboxylate (52.3 mL, 53.7 g, 265 mmol) was added slowly to a mixture of ethyl 1H-pyrazole-5-carboxylate (31.0 g, 221 mmol), (2R)-1-benzyloxypropan-2-ol (44.0 g, 265 mmol), triphenylphosphine (69.6 g, 265 mmol) and molecular sieves (4 Å, 25 g, pre-activated by heating under vacuum for 2 hours) in dry THF (500 mL) at −5° C. under argon. The reaction was stirred at 0° C. for 1 hour, then warmed to room temperature and stirred for 1 hour. Most of the THF (ca. 400 mL) was evaporated, heptane (400 mL) was added to the orange solution under mechanical stirring and the mixture was stirred for 16 hours. The mixture was filtered (to remove the mixture of triphenylphospine oxide and reduced diethyl azodicarboxylate) and the filtrate was concentrated in vacuo. The residue was purified by column chromatography (silica gel, eluting with heptane/EtOAc) to give the title compound as a pale pink oil (45.7 g, 72%). 1H NMR (600 MHz, CDCl₃) δ 7.53 (d, J=1.9 Hz, 1H), 7.34-7.16 (m, 5H), 6.83 (d, J=2.0 Hz, 1H), 5.80-5.63 (m, 1H), 4.46 (d, J=12.2 Hz, 1H), 4.42 (d, J=12.2 Hz, 1H), 4.31 (qd, J=7.1, 1.3 Hz, 2H), 3.85 (dd, J=9.9, 8.0 Hz, 1H), 3.69 (dd, J=9.9, 5.3 Hz, 1H), 1.51 (d, J=6.8 Hz, 3H), 1.35 (t, J=7.1 Hz, 3H); LCMS (METHOD 3) (ES): m/z 289.3 [M+H]⁺, RT=0.84 min.

### Preparation 221: 2-[(1S)-2-Benzyloxy-1-methyl-ethyl]pyrazole-3-carboxylic acid

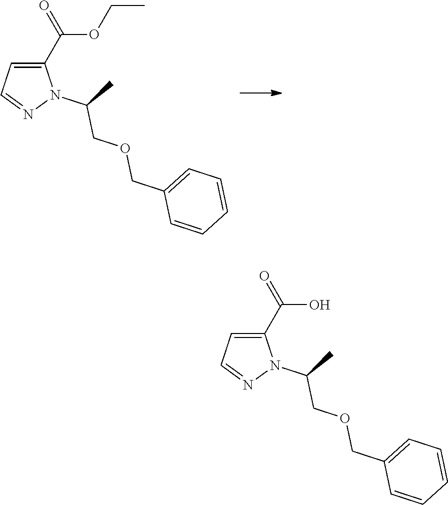 [SMILES: CCOC(=O)c1ccnn1[C@@H](C)COCc1ccccc1 ; C[C@@H](COCc1ccccc1)n1nccc1C(=O)O]

The ester of Preparation 220 (45.74 g, 159 mmol) was dissolved in MeOH (100 mL) and 5M NaOH (40 mL) was added. The mixture was stirred overnight at room temperature. Most of the MeOH was evaporated, the pH was adjusted to 2-3 with 6M aq. hydrogen chloride and the mixture was extracted with TBME (3×100 mL). The combined organic extracts were dried (Na₂SO₄) and evaporated to give the title compound which was used directly without further purification. LCMS (METHOD 3) (ES): m/z 261.2 [M+H]⁺, RT=0.61 min.

### Preparation 222: 2-[(1S)-2-Hydroxy-1-methyl-ethyl]pyrazole-3-carboxylic acid

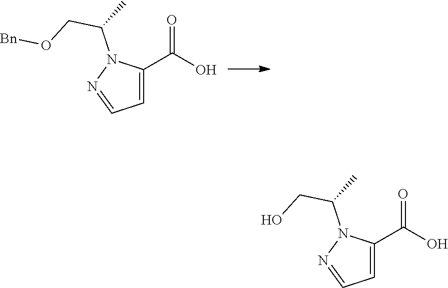 [SMILES: C[C@@H](COCc1ccccc1)n1nccc1C(=O)O ; C[C@@H](CO)n1nccc1C(=O)O]

The acid of Preparation 221 (41.3 g, 159 mmol) was dissolved in MeOH (250 mL) and hydrogenated over 10% Pd/C (2 g) at 1.5 bar on a Parr shaker. Filtration through Celite and evaporation of the filtrate gave the title compound as a white solid (26.8 g, 99%). 1H NMR (600 MHz, DMSO-d6) δ 13.22 (s, 1H), 7.54 (d, J=1.9 Hz, 1H), 6.78 (d, J=1.9 Hz, 1H), 5.58-5.20 (m, 1H), 4.80 (s, 1H), 3.69 (dd, J=10.7, 7.6 Hz, 1H), 3.59 (dd, J=10.7, 5.8 Hz, 1H), 1.35 (d, J=6.7 Hz, 3H); LCMS (METHOD 3) (ES): m/z 171.2 [M+H]⁺, RT=0.27 min.

### Preparation 223: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-[(1S)-2-hydroxy-1-methyl-ethyl]pyrazole-3-carboxamide

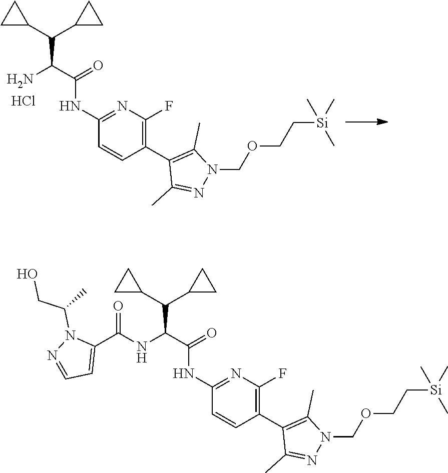 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2ccnn2[C@@H](C)CO)C(C2CC2)C2CC2)nc1F]

According to the method of Preparation 11 the compound of Preparation 92 (15 mg, 0.03 mmol) was reacted with the compound from Preparation 222 (10.3 mg, 0.06 mmol) to afford the title compound after acidic prep. HPLC (5.0 mg, 25% yield). LCMS (METHOD 3) (ES): m/z 640.5 [M+H]⁺, RT=0.92 min.

### Preparation 224: 4-fluoro-1-(3-tetrahydropyran-2-yloxypropyl)pyrazole

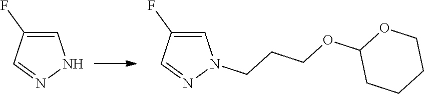 [SMILES: Fc1cn[nH]c1 ; Fc1cnn(CCCOC2CCCCO2)c1]

According to the method of Preparation 196 4-fluoro-1H-pyrazole (1.0 g, 11.6 mmol) was reacted with 2-(3-bromopropoxy)tetrahydropyran (2.59 g, 11.6 mmol) to afford the title compound after silica chromatography (2.21 g, 83% yield). 1H NMR (400 MHz, CDCl₃) δ 7.32 (dd, J=4.3, 0.8 Hz, 1H), 7.29 (dd, J=4.9, 0.8 Hz, 1H), 4.54 (dd, J=4.7, 2.8 Hz, 1H), 4.16 (td, J=6.9, 2.2 Hz, 2H), 3.90-3.80 (m, 1H), 3.79-3.70 (m, 1H), 3.55-3.45 (m, 1H), 3.41-3.30 (m, 1H), 2.16-2.05 (m, 2H), 1.93-1.43 (m, 6H).

### Preparation 225: 4-fluoro-2-(3-tetrahydropyran-2-yloxypropyl)pyrazole-3-carboxylic acid

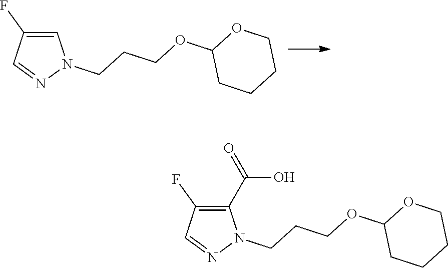 [SMILES: Fc1cnn(CCCOC2CCCCO2)c1 ; O=C(O)c1c(F)cnn1CCCOC1CCCCO1]

n-Butyllithium (2.5 M soln in heptanes, 5.0 mL, 12.5 mmol) was added dropwise to solution of the compound of Preparation 224 (2.20 g, 9.64 mmol) in Et₂O (25 mL) at −10° C. The pale yellow reaction mixture was warmed to room temperature and stirred for 30 minutes. CO₂ (g) was bubbled through the reaction mixture for 20 minutes. The reaction mixture was quenched with H₂O (40 mL) and extracted with Et₂O (2×20 mL). The aqueous phase was then acidified to pH 3 with 1M NaHSO₄ (aq. solution) and extracted with EtOAc (2×20 mL). The combined organic layers were dried over MgSO₄, filtered and concentrated in vacuo. The obtained crude compound was purified by silica column chromatography (230-400 mesh), eluting with EtOAc in heptane, to afford the title compound (1.72 g, 65% yield). 1H NMR (400 MHz, CDCl₃) δ 8.89 (s, 1H), 7.41 (d, J=4.4 Hz, 1H), 4.73-4.50 (m, 3H), 3.86 (ddd, J=11.3, 8.0, 3.1 Hz, 1H), 3.78 (dt, J=10.1, 6.1 Hz, 1H), 3.57-3.49 (m, 1H), 3.41 (dt, J=10.1, 6.2 Hz, 1H), 2.17-2.08 (m, 2H), 1.82 (dddt, J=14.0, 8.3, 5.9, 3.0 Hz, 1H), 1.76-1.65 (m, 1H), 1.64-1.46 (m, 4H); LCMS (METHOD 3) (ES): m/z 271.2 [M−H]⁻, RT=0.52 min.

### Preparation 226: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-4-fluoro-2-(3-tetrahydropyran-2-yloxypropyl)pyrazole-3-carboxamide

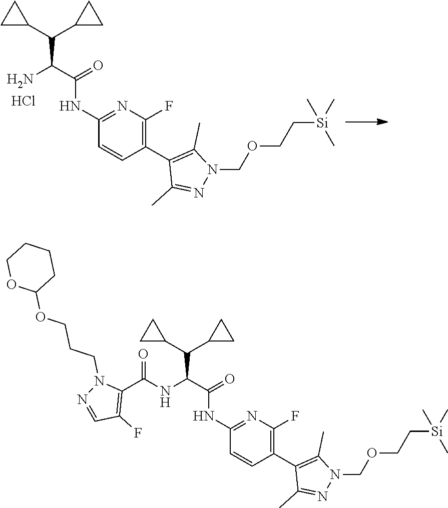 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2c(F)cnn2CCCOC2CCCCO2)C(C2CC2)C2CC2)nc1F]

According to the method of Preparation 11 the compound of Preparation 92 (50 mg, 0.102 mmol) was reacted with the compound from Preparation 225 (27.9 mg, 0.102 mmol) to afford the title compound after acidic prep. HPLC (37 mg, 48% yield). LCMS (METHOD 3) (ES): m/z 742.6 [M+H]⁺, RT=1.07 min.

### Preparation 227: 3-[5-[[(1S)-1-(dicyclopropylmethyl)-2-[[5-(3,5-dimethyl-1H-pyrazol-4-yl)-6-fluoro-2-pyridyl]amino]-2-oxo-ethyl]carbamoyl]-4-fluoro-pyrazol-1-yl]propyl 2,2,2-trifluoroacetate

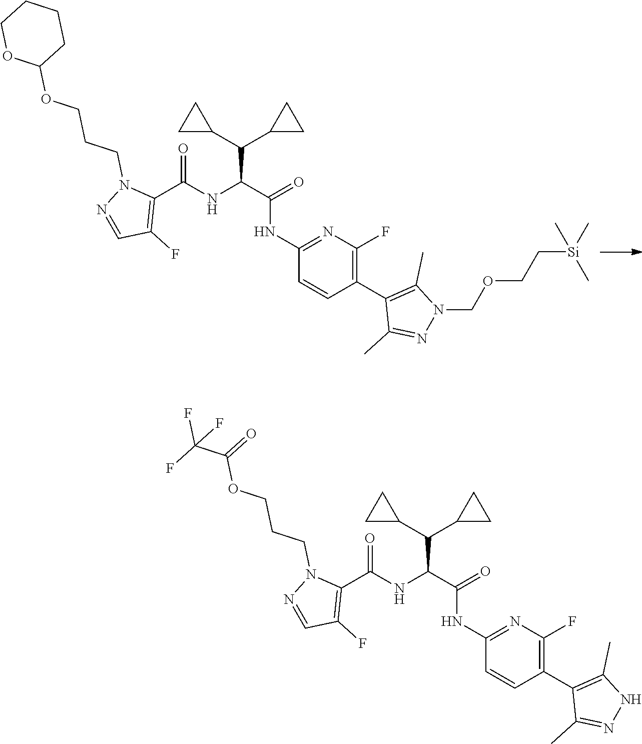 [SMILES: Cc1nn(COCC[Si](C)(C)C)c(C)c1-c1ccc(NC(=O)[C@@H](NC(=O)c2c(F)cnn2CCCOC2CCCCO2)C(C2CC2)C2CC2)nc1F ; Cc1n[nH]c(C)c1-c1ccc(NC(=O)[C@@H](NC(=O)c2c(F)cnn2CCCOC(=O)C(F)(F)F)C(C2CC2)C2CC2)nc1F]

According to the method of Example 1 the compound of Preparation 226 (37 mg, 0.05 mmol) was reacted to afford the crude title compound (32 mg, assume 100% yield). LCMS (METHOD 3) (ES): m/z 624.4 [M+H]⁺, RT=0.85 min.

### Preparation 228: ethyl 2-(2-fluoro-1-methyl-ethyl)pyrazole-3-carboxylate

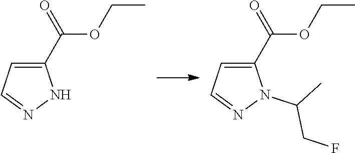

According to the method of Preparation 147, ethyl 1H-pyrazole-5-carboxylate (250 mg, 1.78 mmol) was reacted with 1-fluoropropan-2-ol (195 mg, 2.50 mmol). The obtained crude compound was purified by silica column chromatography (230-400 mesh), eluting with EtOAc in heptane, to afford the title compound as a colourless oil (337 mg, 85% yield). 1H NMR (400 MHz, CDCl₃) δ 7.56 (d, J=2.4 Hz, 1H), 6.89-6.80 (m, 1H), 5.78 (dq, J=13.4, 6.7 Hz, 1H), 4.91-4.70 (m, 1H), 4.70-4.46 (m, 1H), 4.43-4.28 (m, 2H), 1.53-1.49 (m, 3H), 1.41-1.35 (m, 3H).

### Preparation 229: 2-(2-fluoro-1-methyl-ethyl)pyrazole-3-carboxylic acid

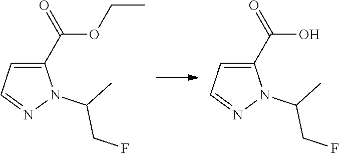

According to the method of Preparation 148, the compound of Preparation 228 (304 mg, 1.52 mmol) was reacted to afford the crude title compound (260 mg, assume 100% yield). 1H NMR (400 MHz, CDCl₃) δ 7.60 (d, J=2.5 Hz, 1H), 6.97 (d, J=2.5 Hz, 1H), 5.77 (dq, J=13.3, 6.8 Hz, 1H), 4.90-4.70 (m, 1H), 4.60 (ddd, J=46.5, 9.8, 5.0 Hz, 1H), 1.53 (dd, J=7.4, 2.2 Hz, 3H); LCMS (METHOD 3) (ES): m/z 173.2 [M+H]⁺, RT=0.38 min.

### Preparation 230: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(2-fluoro-1-methyl-ethyl)pyrazole-3-carboxamide

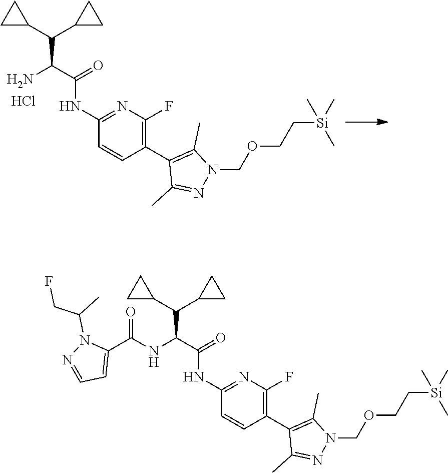 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2ccnn2C(C)CF)C(C2CC2)C2CC2)nc1F]

According to the method of Preparation 11 the compound of Preparation 92 (50 mg, 0.10 mmol) was reacted with the compound from Preparation 229 (24.6 mg, 0.14 mmol) to afford the title compound (37 mg, 56% yield). LCMS (METHOD 3) (ES): m/z 642.5 [M+H]⁺, RT=0.97 min.

### Preparation 231: ethyl 2-(2,2-difluoro-1-methyl-ethyl)pyrazole-3-carboxylate

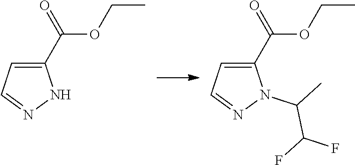

According to the method of Preparation 147, ethyl 1H-pyrazole-5-carboxylate (300 mg, 2.14 mmol) was reacted with 1,1-difluoropropan-2-ol (246 mg, 2.57 mmol). The obtained crude compound was purified by silica column chromatography (230-400 mesh), eluting with EtOAc in heptane, to afford the title compound as a colourless oil (388 mg, 77% yield). 1H NMR (400 MHz, CDCl₃) δ 7.57 (q, J=1.8 Hz, 1H), 6.87 (q, J=1.8 Hz, 1H), 6.23-5.85 (m, 1H), 5.76 (ddd, J=14.2, 10.7, 7.5 Hz, 1H), 4.44-4.27 (m, 2H), 1.64 (dd, J=7.2, 2.0 Hz, 3H), 1.39 (td, J=7.2, 3.6 Hz, 3H); LCMS (METHOD 3) (ES): m/z 219.2 [M+H]⁺, RT=0.72 min.

### Preparation 232: 2-(2,2-difluoro-1-methyl-ethyl)pyrazole-3-carboxylic acid

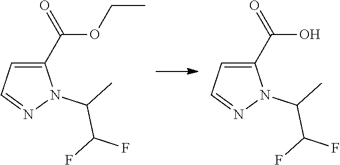

According to the method of Preparation 148, the compound of Preparation 231 (388 mg, 1.78 mmol) was reacted to afford the crude title compound (305 mg, 90% yield). 1H NMR (400 MHz, CDCl₃) δ 7.63 (q, J=1.9 Hz, 1H), 7.00 (d, J=2.4 Hz, 1H), 6.26-5.86 (m, 1H), 5.72 (q, J=8.0 Hz, 1H), 1.67 (s, 4H); LCMS (METHOD 3) (ES): m/z 191.2 [M+H]⁺, RT=0.41 min.

### Preparation 233: N-[(1S)-1-(dicyclopropylmethyl)-2-[[5-[3,5-dimethyl-1-(2-trimethylsilylethoxymethyl)pyrazol-4-yl]-6-fluoro-2-pyridyl]amino]-2-oxo-ethyl]-2-(2,2-difluoro-1-methyl-ethyl)pyrazole-3-carboxamide

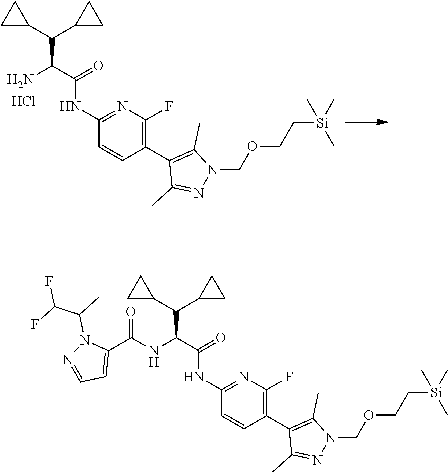 [SMILES: Cc1nn(COCC[Si](C)(C)C)c(C)c1-c1ccc(NC(=O)[C@@H](N)C(C2CC2)C2CC2)nc1F ; Cl ; Cc1nn(COCC[Si](C)(C)C)c(C)c1-c1ccc(NC(=O)[C@@H](NC(=O)c2ccnn2C(C)C(F)F)C(C2CC2)C2CC2)nc1F]

According to the method of Preparation 11 the compound of Preparation 92 (50 mg, 0.10 mmol) was reacted with the compound from Preparation 232 (27.2 mg, 0.14 mmol) to afford the title compound (65 mg, assume 100% yield). LCMS (METHOD 3) (ES): m/z 642.5 [M+H]⁺, RT=0.97 min.

### Preparation 234: 5-(3,5-dimethylisoxazol-4-yl)-6-fluoro-pyridin-2-amine

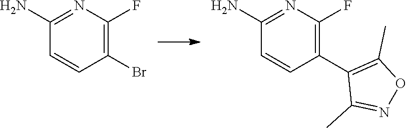

According to the method of Preparation 22, 5-bromo-6-fluoro-pyridin-2-amine (500 mg, 2.62 mmol) was reacted with 3,5-dimethyl-4-(4,4,5,5-tetramethyl-1,3,2-dioxaborolan-2-yl)isoxazole (643 mg, 2.88 mmol) to afford the title compound after flash chromatography, as a tan oil (450 mg, 83% yield). 1H NMR (300 MHz, DMSO-d6) δ 7.48 (dd, J=8.1, 10.3 Hz, 1H), 6.52 (s, 2H), 6.41 (dd, J=2.2, 8.1 Hz, 1H), 2.28 (s, 3H), 2.11 (s, 3H); LCMS (METHOD 2) (ESI): m/z: 208 [M+H]⁺; 89%; RT=2.44 min (ACQUITY BEH C18 column, 0.05% FA in water with MeCN).

### Preparation 235: N-[(1S)-1-[(5-bromo-6-fluoro-2-pyridyl)carbamoyl]-2,2-dicyclopropyl-ethyl]-2-ethyl-pyrazole-3-carboxamide

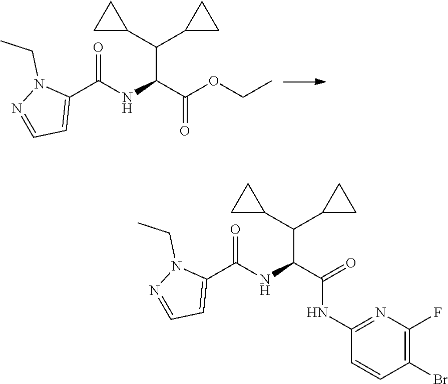

According to the method of Preparation 27 the compound of Preparation 44 (400 mg, 1.31 mmol) was reacted with 5-bromo-6-fluoro-pyridin-2-amine (249 mg, 1.31 mmol) to afford the title compound as an off-white solid (400 mg, 65% yield). 1H NMR (300 MHz, DMSO-d6) δ 11.1 (br s, 1H), 8.48 (d, J=8.4 Hz, 1H), 8.24 (t, J=9 Hz, 1H), 7.98 (dd, J=0.9, 8.4 Hz, 1H), 7.49 (d, J=1.8 Hz, 1H), 6.99 (d, J=1.8 Hz, 1H), 4.87 (t, J=7.8 Hz, 1H), 4.46 (q, J=6.8 Hz, 2H), 1.27 (t, J=6.9 Hz, 3H), 0.8-0.6 (m, 3H), 0.5-0.2 (m, 8H); LCMS (METHOD 2) (ESI): m/z: 464 [M+H]⁺; 90%; RT=2.18 min (ACQUITY BEH C18 column, 0.1% FA in water with MeCN).

### Preparation 236: N-[(1S)-1-(dicyclopropylmethyl)-2-[[6-fluoro-5-(4,4,5,5-tetramethyl-1,3,2-dioxaborolan-2-yl)-2-pyridyl]amino]-2-oxo-ethyl]-2-ethyl-pyrazole-3-carboxamide

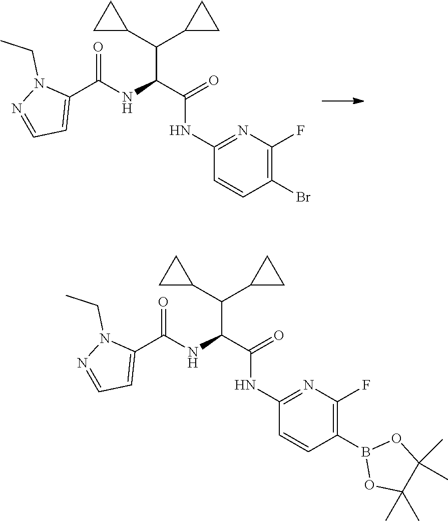 [SMILES: CCn1nccc1C(=O)N[C@H](C(=O)Nc1ccc(Br)c(F)n1)C(C1CC1)C1CC1 ; CCn1nccc1C(=O)N[C@H](C(=O)Nc1ccc(B2OC(C)(C)C(C)(C)O2)c(F)n1)C(C1CC1)C1CC1]

KOAc (84 mg, 0.86 mmol) was added to a solution of the compound of Preparation 235 (100 mg, 0.22 mmol) and bis(pinacolato)diboron (82 mg, 0.86 mmol) in 1,4-dioxane (5 mL). The reaction mixture was purged with argon for 10 mins before Pd(dppf)Cl₂·DCM (18 mg, 0.021 mmol) was added and the reaction mixture was stirred at 110° C. for 2.5 hours. The cooled reaction mixture was filtered through Celite, washing with EtOAc (40 mL). The filtrate was dried over Na₂SO₄, filtered and concentrated in vacuo to afford the crude title compound (190 mg, assume 100% yield). LCMS (METHOD 2) (ESI): m/z: 430 [M+H]⁺; 42% of boronic acid & m/z: 512 [M+H]⁺; 12% of boronic ester; RT=2.18 min & RT=2.80 (ACQUITY BEH C18 column, 0.05% FA in water with MeCN).

### Preparation 237: (2,3,4,5,6-pentafluorophenyl) (2S)-2-(tert-butoxycarbonylamino)-3,3-dicyclopropyl-propanoate

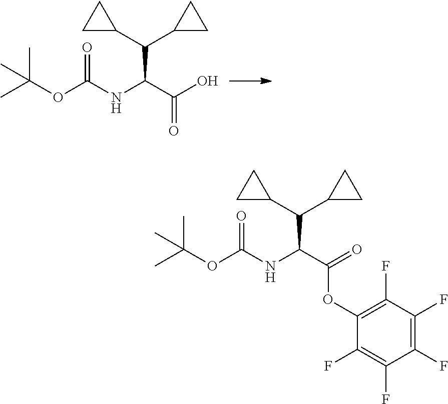 [SMILES: CC(C)(C)OC(=O)N[C@H](C(=O)O)C(C1CC1)C1CC1 ; CC(C)(C)OC(=O)N[C@H](C(=O)Oc1c(F)c(F)c(F)c(F)c1F)C(C1CC1)C1CC1]

(2,3,4,5,6-pentafluorophenyl) 2,2,2-trifluoroacetate (6.2 g, 22.0 mmol) was added to a solution of the compound of Preparation 36 (5.0 g, 19.0 mmol) and pyridine (5.0 mL, 62.1 mmol) in DCM (100 mL) at room temperature and the reaction mixture was stirred for 16 hours. The reaction mixture was washed successively with 1M hydrogen chloride (aq, 30 mL) and saturated aq. NaHCO₃ (30 mL). The organic phase was dried over MgSO₄, filtered and concentrated in vacuo. The obtained crude compound was purified by silica column chromatography (230-400 mesh), eluting with EtOAc in heptane, to afford the title compound (6.72 g, 83% yield). 1H NMR (400 MHz, CDCl₃) δ 5.33 (d, J=9.3 Hz, 1H), 4.87 (d, J=8.8 Hz, 1H), 1.48 (s, 9H), 0.94-0.73 (m, 2H), 0.73-0.42 (m, 4H), 0.42-0.16 (m, 4H).

### Preparation 238: 2-[[3,5-dimethyl-4-(2,4,6-trifluoro-3-pyridyl)pyrazol-1-yl]methoxy]ethyl-trimethyl-silane

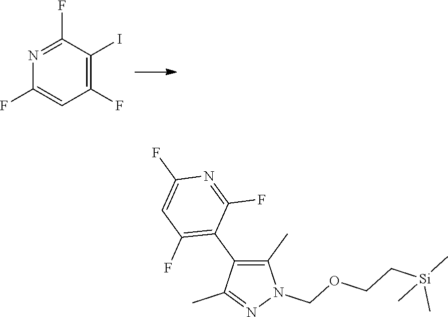 [SMILES: Fc1cc(F)c(I)c(F)n1 ; Cc1nn(COCC[Si](C)(C)C)c(C)c1-c1c(F)cc(F)nc1F]

According to the method of Preparation 22 2,4,6-trifluoro-3-iodo-pyridine (2.5 g, 9.7 mmol) was reacted with the compound from Preparation 21 (4.1 g, 12.0 mmol) to afford the title compound after silica chromatography (2.6 g, 30% yield). LCMS (METHOD 3) (ES): m/z 358.2 [M+H]⁺, RT=0.94 min.

### Preparation 239: 5-[3,5-dimethyl-1-(2-trimethylsilylethoxymethyl)pyrazol-4-yl]-4,6-difluoro-pyridin-2-amine

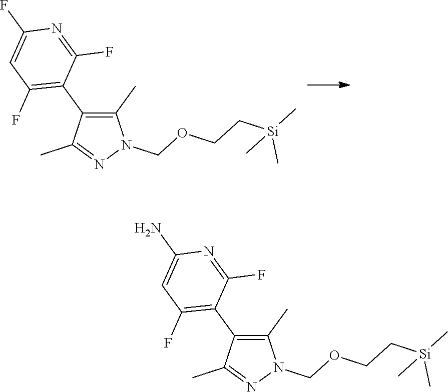 [SMILES: Cc1nn(COCC[Si](C)(C)C)c(C)c1-c1c(F)cc(F)nc1F ; Cc1nn(COCC[Si](C)(C)C)c(C)c1-c1c(F)cc(N)nc1F]

Ammonium hydroxide (0.5 mL) was added to a solution of the compound of Preparation 238 (1.0 g, 1.12 mmol) in DMSO (10 mL) and stirred at 100° C. for 30 minutes. The cooled reaction mixture was diluted with H₂O (40 mL) and extracted with TBME (3×30 mL). The combined organic phase was dried over MgSO₄, filtered and concentrated in vacuo. the obtained crude compound was purified by silica column chromatography (230-400 mesh), eluting with EtOAc in heptane, to afford the title compound (0.11 g, 27% yield). 1H NMR (400 MHz, CDCl₃) δ 6.19 (dd, J=9.4, 2.7 Hz, 1H), 5.40 (t, J=2.3 Hz, 2H), 4.71 (s, 2H), 3.70-3.55 (m, 2H), 2.22 (s, 3H), 2.16 (s, 3H), 0.92 (dt, J=9.3, 4.7 Hz, 2H) 0.00 (s, 9H); LCMS (METHOD 3) (ES): m/z 355.3 [M+H]⁺, RT=0.81 min.

### Preparation 240: tert-butyl N-[(1S)-1-(dicyclopropylmethyl)-2-[[5-[3,5-dimethyl-1-(2-trimethylsilylethoxymethyl)pyrazol-4-yl]-4,6-difluoro-2-pyridyl]amino]-2-oxo-ethyl]carbamate

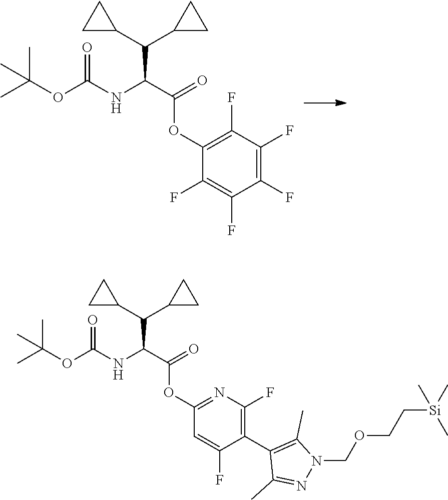 [SMILES: CC(C)(C)OC(=O)N[C@H](C(=O)Oc1c(F)c(F)c(F)c(F)c1F)C(C1CC1)C1CC1 ; Cc1nn(COCC[Si](C)(C)C)c(C)c1-c1c(F)cc(OC(=O)[C@@H](NC(=O)OC(C)(C)C)C(C2CC2)C2CC2)nc1F]

Tert-Butylmagnesium chloride (1.0 M in THF, 1.25 mL) was added to a solution of the compound of Preparation 239 (110.0 mg, 0.25 mmol) and the compound of Preparation 237 (90.0 mg, 0.25 mmol) in THF (5 mL) at 5° C. The reaction mixture was stirred for 1 hour at 5° C. The reaction mixture was quenched with saturated aq. NH₄Cl (10 mL) and extracted with TBME (2×10 mL). The combined organic phase was dried over MgSO₄, filtered and concentrated in vacuo. The obtained crude compound was purified by prep. acidic HPLC to afford the title compound as a colourless oil (75.0 mg, 48% yield). 1H NMR (600 MHz, CDCl₃) δ 8.63 (s, 1H), 8.04 (d, J=9.6 Hz, 1H), 5.41 (d, J=3.5 Hz, 2H), 5.34 (s, 1H), 4.47 (s, 1H), 3.63 (ddd, J=9.9, 7.9, 2.0 Hz, 2H), 2.23 (d, J=4.9 Hz, 3H), 2.17 (d, J=2.1 Hz, 3H), 1.50 (d, J=3.6 Hz, 9H), 1.04-0.85 (m, 3H), 0.83-0.67 (m, 2H), 0.60 (q, J=8.1, 5.9 Hz, 2H), 0.57-0.45 (m, 2H), 0.39-0.20 (m, 4H), 0.00 (s, 9H); LCMS (METHOD 3) (ES): m/z 606.5 [M+H]⁺, RT=1.05 min.

### Preparation 241: (2S)-2-amino-3,3-dicyclopropyl-N-[5-(3,5-dimethyl-1H-pyrazol-4-yl)-4,6-difluoro-2-pyridyl]propenamide hydrochloride

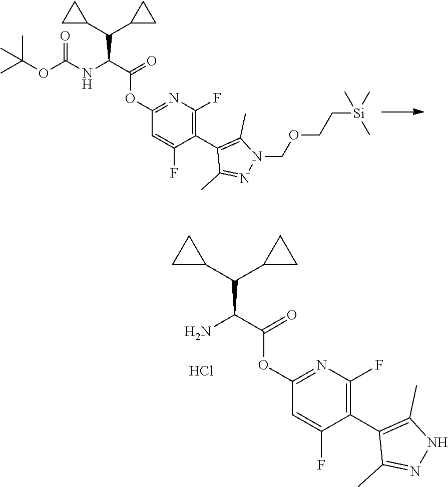 [SMILES: Cc1nn(COCC[Si](C)(C)C)c(C)c1-c1c(F)cc(OC(=O)[C@@H](NC(=O)OC(C)(C)C)C(C2CC2)C2CC2)nc1F ; Cc1n[nH]c(C)c1-c1c(F)cc(OC(=O)[C@@H](N)C(C2CC2)C2CC2)nc1F ; Cl]

Hydrogen chloride (4M soln in dioxane, 1.0 mL) was added to a solution of the compound of Preparation 240 (60.0 mg, 0.099 mmol) in DCM (2 mL) and stirred at room temperature for 4 hours. The reaction mixture was stored at 0° C. for 64 hours, then concentrated in vacuo to leave crude title compound as a colourless solid. (40 mg, assume 100% yield). The product was used directly without characterisation.

### Preparation 242: tert-butyl N-[(1S)-1-[[5-bromo-4-(difluoromethyl)-2-pyridyl]carbamoyl]-2,2-dicyclopropyl-ethyl]carbamate

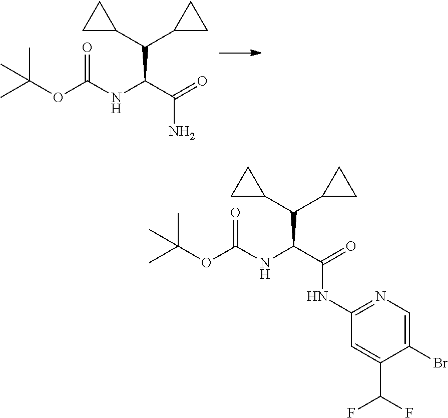 [SMILES: CC(C)(C)OC(=O)N[C@H](C(N)=O)C(C1CC1)C1CC1 ; CC(C)(C)OC(=O)N[C@H](C(=O)Nc1cc(C(F)F)c(Br)cn1)C(C1CC1)C1CC1]

According to the method of Preparation 90 the compound of Preparation 89 (100 mg, 0.37 mmol) was reacted with 2,5-dibromo-4-(difluoromethyl)pyridine (112 mg, 0.39 mmol) to afford the title compound after silica chromatography (144 mg, 81% yield). 1H NMR (400 MHz, CDCl₃) δ 8.90 (s, 1H), 8.51 (s, 1H), 8.42 (s, 1H), 6.77 (t, J=54.1 Hz, 1H), 5.46 (d, J=8.2 Hz, 1H), 4.50 (s, 1H), 1.46 (s, 9H), 0.88 (td, J=7.2, 6.2, 2.6 Hz, 2H), 0.75 (dddd, J=16.9, 8.5, 5.0, 2.6 Hz, 2H), 0.64-0.37 (m, 4H), 0.37-0.15 (m, 4H); LCMS (METHOD 3) (ES): m/z 472.3 [M−H]⁻, RT=0.92 min.

### Preparation 243: tert-butyl N-[(1S)-1-(dicyclopropylmethyl)-2-[[4-(difluoromethyl)-5-[3,5-dimethyl-1-(2-trimethylsilylethoxymethyl)pyrazol-4-yl]-2-pyridyl]amino]-2-oxo-ethyl]carbamate

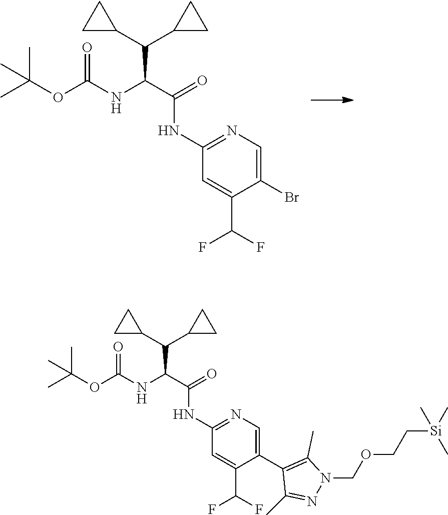 [SMILES: CC(C)(C)OC(=O)N[C@H](C(=O)Nc1cc(C(F)F)c(Br)cn1)C(C1CC1)C1CC1 ; Cc1nn(COCC[Si](C)(C)C)c(C)c1-c1cnc(NC(=O)[C@@H](NC(=O)OC(C)(C)C)C(C2CC2)C2CC2)cc1C(F)F]

According to the method of Preparation 7 the compound of Preparation 242 (50.0 mg, 0.11 mmol) was reacted with the compound of Preparation 41 (55.7 mg, 0.16 mmol). The crude material was purified by prep. acidic HPLC to afford the title compound (41 mg, 62% yield). LCMS (METHOD 3) (ES): m/z 620.6 [M+H]⁺, RT=1.03 min.

### Preparation 244: (2S)-2-amino-3,3-dicyclopropyl-N-[4-(difluoromethyl)-5-[3,5-dimethyl-1-(2-trimethylsilylethoxymethyl)pyrazol-4-yl]-2-pyridyl]propenamide

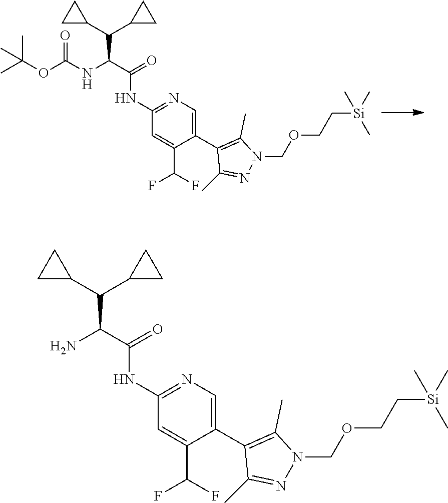 [SMILES: Cc1nn(COCC[Si](C)(C)C)c(C)c1-c1cnc(NC(=O)[C@@H](NC(=O)OC(C)(C)C)C(C2CC2)C2CC2)cc1C(F)F ; Cc1nn(COCC[Si](C)(C)C)c(C)c1-c1cnc(NC(=O)[C@@H](N)C(C2CC2)C2CC2)cc1C(F)F]

Hydrogen chloride (4M soln in dioxane, 2.0 mL) was added to a solution of the compound of Preparation 243 (41.0 mg, 0.066 mmol) in MeOH (1 mL) and stirred at room temperature for 40 minutes. MeOH (2 mL) was added and the reaction mixture was concentrated in vacuo to leave crude title compound (34 mg, assume 100% yield). LCMS (METHOD 3) (ES): m/z 520.5 [M+H]⁺, RT=0.82 min.

### Preparation 245: N-[(1S)-1-(dicyclopropylmethyl)-2-[[4-(difluoromethyl)-5-[3,5-dimethyl-1-(2-trimethylsilylethoxymethyl)pyrazol-4-yl]-2-pyridyl]amino]-2-oxo-ethyl]-2-ethyl-pyrazole-3-carboxamide

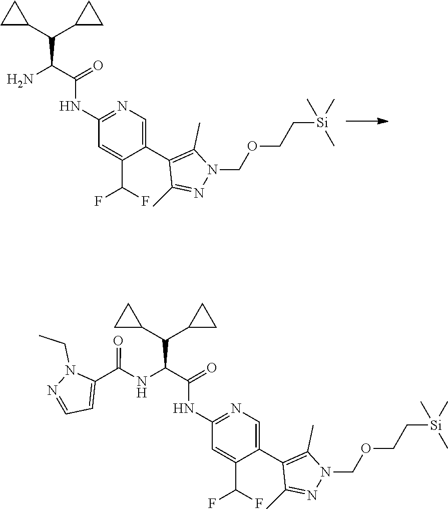

According to the method of Preparation 11 the compound of Preparation 244 (17 mg, 0.033 mmol) was reacted with 2-ethylpyrazole-3-carboxylic acid (4.6 mg, 0.033 mmol) to afford the title compound after acidic prep. HPLC (15.0 mg, 71% yield). LCMS (METHOD 3) (ES): m/z 642.5 [M+H]⁺, RT=0.98 min.

### Preparation 246: N-[(1S)-1-(dicyclopropylmethyl)-2-[[4-(difluoromethyl)-5-[3,5-dimethyl-1-(2-trimethylsilylethoxymethyl)pyrazol-4-yl]-2-pyridyl]amino]-2-oxo-ethyl]-2-isopropyl-pyrazole-3-carboxamide

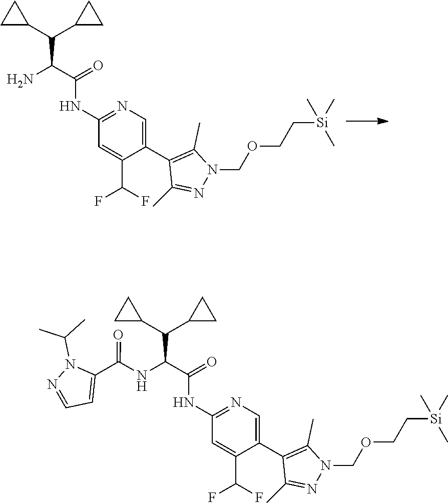

According to the method of Preparation 11 the compound of Preparation 244 (17 mg, 0.033 mmol) was reacted with 2-isopropylpyrazole-3-carboxylic acid (5.1 mg, 0.033 mmol) to afford the title compound after prep. acidic HPLC (15.0 mg, 69% yield). LCMS (METHOD 3) (ES): m/z 656.5 [M+H]⁺, RT=1.00 min.

### Preparation 247: methyl 6-[bis(tert-butoxycarbonyl)amino]-3-bromo-pyridine-2-carboxylate

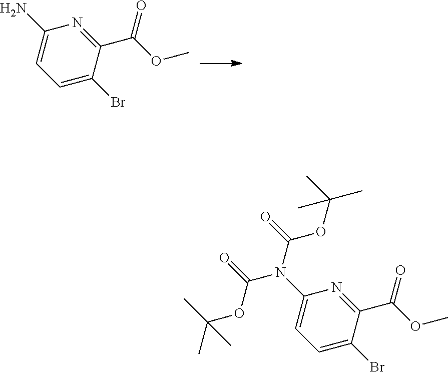 [SMILES: COC(=O)c1nc(N)ccc1Br ; COC(=O)c1nc(N(C(=O)OC(C)(C)C)C(=O)OC(C)(C)C)ccc1Br]

DMAP (50 mg, 0.41 mmol) was added to a solution of methyl 6-amino-3-bromo-pyridine-2-carboxylate (1.5 g, 6.5 mmol) and tert-butoxycarbonyl tert-butyl carbonate (5.70 g, 26.0 mmol) in ^{t}BuOH (30 mL) and acetone (7.5 mL) at room temperature. After 18 hours the reaction mixture was concentrated in vacuo and the obtained crude compound was purified by silica column chromatography (230-400 mesh), eluting with EtOAc in heptane, to afford the title compound as a colourless solid (2.5 g, 81% yield). 1H NMR (400 MHz, CDCl₃) δ 7.98 (d, J=8.6 Hz, 1H), 7.33 (d, J=8.6 Hz, 1H), 3.96 (s, 3H), 1.49 (s, 18H).

### Preparation 248: tert-butyl N-(5-bromo-6-formyl-2-pyridyl)-N-tert-butoxycarbonyl-carbamate and tert-butyl N-(5-bromo-6-formyl-2-pyridyl)carbamate

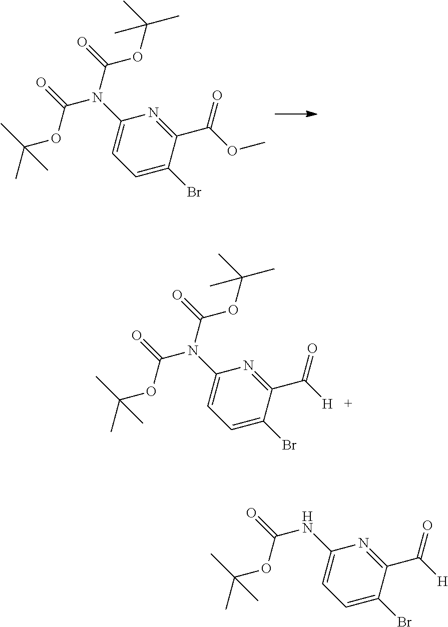

DIBAL (1M soln in toluene, 12.0 mL) was added slowly to a solution of the compound of Preparation 247 (2.5 g, 5.8 mmol) in DCM (40 mL) at −78° C. The reaction mixture was stirred at −78° C. for 3 hours. The reaction mixture was quenched upon addition of MeOH (5 mL) and saturated aq. potassium sodium tartrate (50 mL). The reaction mixture was washed with DCM (2×50 mL). The combined organic phase was dried over Na₂SO₄, filtered and concentrated in vacuo. The obtained crude compound was purified by silica column chromatography (230-400 mesh), eluting with EtOAc in heptane, to afford the mix of title compounds (1.9 g). Carried forward to next step as a mixture.

### Preparation 249: tert-butyl N-[5-bromo-6-(difluoromethyl)-2-pyridyl]-N-tert-butoxycarbonyl-carbamate and tert-butyl N-[5-bromo-6-(difluoromethyl)-2-pyridyl]carbamate

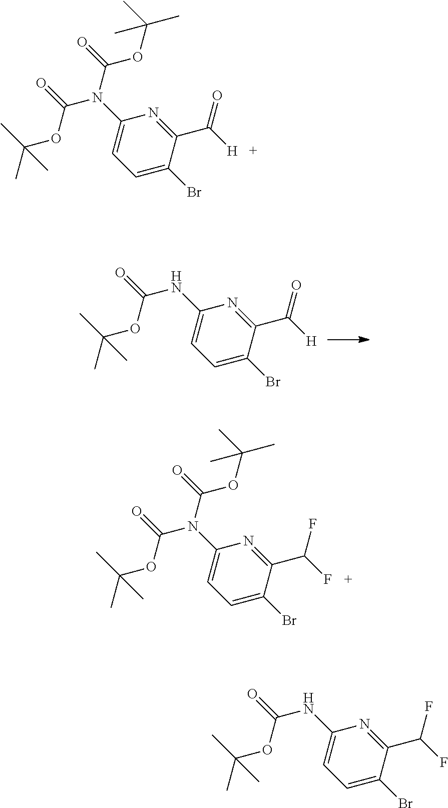

DAST (2.5 ml, 19.0 mmol) was added to a solution of the compounds from Preparation 248 (1.9 g) in DCM (20 mL) at 5° C. The reaction mixture was stirred to room temperature over 2 hours. The reaction mixture was quenched upon careful addition of saturated aq. NaHCO₃ until no gas evolution. The reaction mixture was extracted with DCM (2×50 mL). The combined organic phase was dried over Na₂SO₄, filtered and concentrated in vacuo. The obtained crude compound was purified by silica column chromatography (230-400 mesh), eluting with EtOAc in heptane, to afford the title compounds as colourless oils;

tert-butyl N-[5-bromo-6-(difluoromethyl)-2-pyridyl]carbamate (340 mg, 1.05 mmol). 1H NMR (600 MHz, CDCl₃) δ 8.02-7.95 (m, 1H), 7.86 (dd, J=8.8, 0.9 Hz, 1H), 7.39 (s, 1H), 6.81 (t, J=53.9 Hz, 1H), 1.52 (s, 9H).

tert-butyl N-[5-bromo-6-(difluoromethyl)-2-pyridyl]-N-tert-butoxycarbonyl-carbamate (280 mg, 0.66 mmol). 1H NMR (600 MHz, CDCl₃) δ 7.96 (d, J=8.5 Hz, 1H), 7.40 (dt, J=8.6, 1.0 Hz, 1H), 6.81 (t, J=53.8 Hz, 1H), 1.48 (s, 18H).

### Preparation 250: 6-(difluoromethyl)-5-[3,5-dimethyl-1-(2-trimethylsilylethoxymethyl)pyrazol-4-yl]pyridin-2-amine

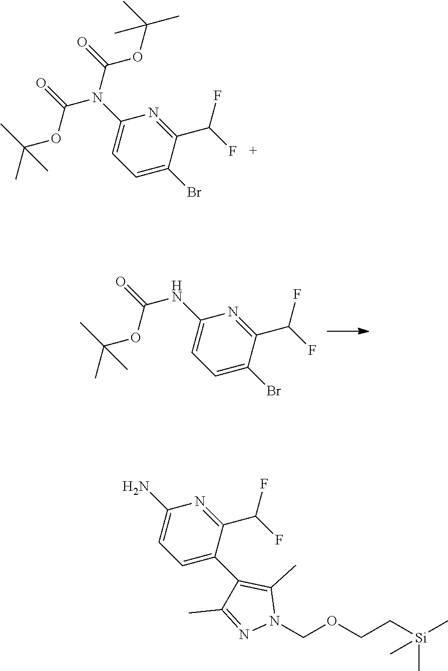

According to the method of Preparation 7 the compounds of Preparation 249 (340 mg, 1.05 mmol and 280 mg, 0.66 mmol) were reacted with the compound of Preparation 41 (800 mg, 2.27 mmol). The organic phase was decanted and the solid washed with TBME (2×25 mL). The combined organic phase was dried over Na₂SO₄, filtered and concentrated in vacuo. The obtained crude compound was purified by silica column chromatography (230-400 mesh), eluting with EtOAc in heptane, to afford the intermediate compounds, tert-butyl N-[6-(difluoromethyl)-5-[3,5-dimethyl-1-(2-trimethylsilylethoxymethyl)pyrazol-4-yl]-2-pyridyl]carbamate and tert-butyl N-tert-butoxycarbonyl-N-[6-(difluoromethyl)-5-[3,5-dimethyl-1-(2-trimethylsilylethoxymethyl)pyrazol-4-yl]-2-pyridyl]carbamate as a colourless oil. The intermediate compounds were dissolved in DCM (5 mL) and hydrogen chloride (4M solution in dioxane, 5.0 mL) was added. The reaction mixture was stirred for 2 hours at room temperature then concentrated in vacuo and purified by silica column chromatography (230-400 mesh), eluting with EtOAc in heptane, to afford the title compound as a colourless oil (231 mg, 28% yield). 1H NMR (400 MHz, CDCl₃) δ 7.27 (dt, J=8.4, 1.1 Hz, 1H), 6.66 (dt, J=8.4, 1.2 Hz, 1H), 6.32 (t, J=54.6 Hz, 1H), 5.39 (s, 2H), 4.96 (s, 1H), 3.68-3.51 (m, 2H), 2.15 (s, 3H), 2.07 (s, 3H), 1.00-0.84 (m, 2H), 0.00 (s, 9H); LCMS (METHOD 3) (ES): m/z 369.3 [M+H]⁺, RT=0.80 min.

### Preparation 251: (2,5-dioxopyrrolidin-1-yl) (2S)-2-(tert-butoxycarbonylamino)-3,3-dicyclopropyl-propanoate

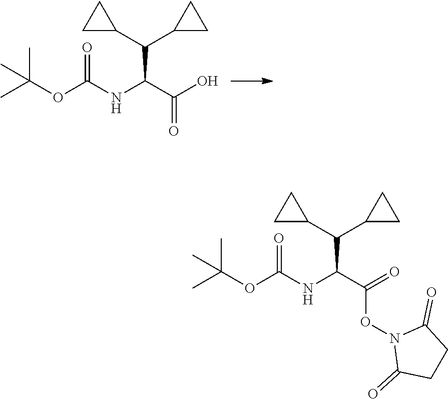 [SMILES: CC(C)(C)OC(=O)N[C@H](C(=O)O)C(C1CC1)C1CC1 ; CC(C)(C)OC(=O)N[C@H](C(=O)ON1C(=O)CCC1=O)C(C1CC1)C1CC1]

EDC (461 mg, 2.41 mmol) was added to a solution of the compound of Preparation 36 (540 mg, 2.00 mmol) and 1-hydroxypyrrolidine-2,5-dione (461 mg, 4.01 mmol) in DCM (10 mL). The reaction mixture was stirred for 18 hours at room temperature then concentrated in vacuo and purified by silica column chromatography (230-400 mesh), eluting with EtOAc in heptane, to afford the title compound as a colourless solid (560 mg, 76% yield). 1H NMR (400 MHz, CDCl₃) δ 5.42-5.01 (m, 0.5H), 5.01-4.60 (m, 0.5H), 2.84 (s, 4H), 1.46 (s, 9H), 0.98-0.74 (m, 4H), 0.69-0.36 (m, 5H), 0.27 (ddd, J=26.9, 9.5, 4.5 Hz, 3H).

### Preparation 252: tert-butyl N-[(1S)-1-(dicyclopropylmethyl)-2-[[6-(difluoromethyl)-5-[3,5-dimethyl-1-(2-trimethylsilylethoxymethyl)pyrazol-4-yl]-2-pyridyl]amino]-2-oxo-ethyl]carbamate

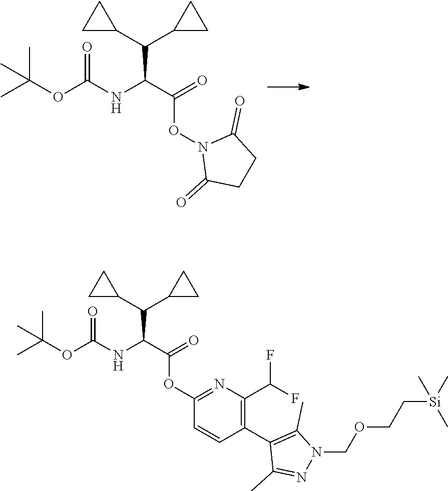 [SMILES: CC(C)(C)OC(=O)N[C@H](C(=O)ON1C(=O)CCC1=O)C(C1CC1)C1CC1 ; Cc1nn(COCC[Si](C)(C)C)c(C)c1-c1ccc(OC(=O)[C@@H](NC(=O)OC(C)(C)C)C(C2CC2)C2CC2)nc1C(F)F]

Tert-butylmagnesium bromide (1 M soln in THF, 1.0 mL) was added to a solution of the compound of Preparation 250 (111 mg, 0.3 mmol) at 5 C. The reaction mixture was stirred at room temperature for 10 minutes then the compound of Preparation 251 (110 mg, 0.3 mmol) was added. The reaction mixture was then stirred at room temperature for a further 30 minutes. The reaction mixture was quenched with saturated aq. NH₄Cl (15 mL). The mixture was diluted with H₂O (15 mL) and extracted with Et₂O (2×20 mL). The combined organic phase was dried over MgSO₄, filtered and concentrated in vacuo. The obtained crude compound was purified by prep. acidic HPLC to afford the title compound as a colourless oil (36.0 mg, 19% yield). 1H NMR (600 MHz, CDCl₃) δ 8.74 (d, J=6.4 Hz, 1H), 8.41 (d, J=8.5 Hz, 1H), 7.58 (d, J=8.4 Hz, 1H), 6.40 (t, J=54.3 Hz, 1H), 5.40 (s, 3H), 4.51 (s, 1H), 3.66-3.60 (m, 2H), 2.15 (d, J=2.6 Hz, 3H), 2.07 (d, J=2.6 Hz, 3H), 1.49 (d, J=1.0 Hz, 9H), 1.00-0.88 (m, 3H), 0.76 (ddd, J=10.2, 8.4, 5.0 Hz, 2H), 0.58 (ddt, J=11.5, 8.0, 3.7 Hz, 2H), 0.55-0.43 (m, 2H), 0.35-0.23 (m, 4H), 0.00 (s, 9H); LCMS (METHOD 3) (ES): m/z 620.5 [M+H]⁺, RT=1.04 min.

### Preparation 253: (2S)-2-amino-3,3-dicyclopropyl-N-[6-(difluoromethyl)-5-(3,5-dimethyl-1H-pyrazol-4-yl)-2-pyridyl]propenamide

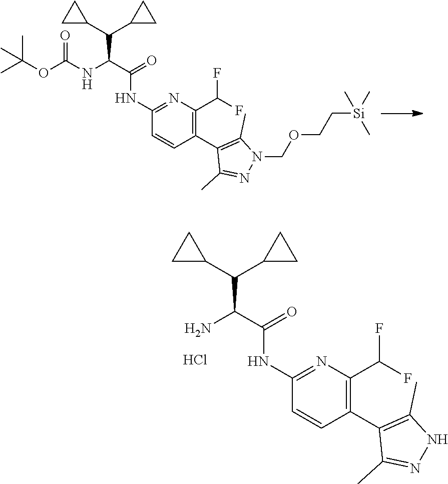 [SMILES: Cc1nn(COCC[Si](C)(C)C)c(C)c1-c1ccc(NC(=O)[C@@H](NC(=O)OC(C)(C)C)C(C2CC2)C2CC2)nc1C(F)F ; Cc1n[nH]c(C)c1-c1ccc(NC(=O)[C@@H](N)C(C2CC2)C2CC2)nc1C(F)F ; Cl]

Hydrogen chloride (4M soln in dioxane, 1.0 mL) was added to a solution of the compound of Preparation 252 (36.0 mg, 0.058 mmol) in DCM (2 mL) and stirred at room temperature for 2 hours. The reaction mixture was concentrated in vacuo to leave crude title compound as a colourless solid. (25 mg, assume 100% yield). LCMS (METHOD 3) (ES): m/z 390.3 [M+H]⁺, RT=0.52 min.

### Preparation 254: 3-bromopyridine-2,6-diamine

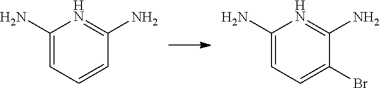

Benzyltrimethylammonium tribromide (4.28 g, 11.0 mmol) was added portionwise to a solution of pyridine-2,6-diamine (1.09 g, 9.99 mmol) in MeOH (5 mL) and stirred at room temperature for 30 minutes. The reaction mixture was diluted with H₂O (50 mL) and the pH was adjusted to 8 with K₂CO₃. The resulting precipitate was filtered, washing with DCM (50 mL). The filtrate was separated, the organic phase was washed with H₂O (20 mL), dried over MgSO₄, filtered and concentrated in vacuo, to leave the title compound as an off-white solid. (749 mg, 40% yield). LCMS (METHOD 3) (ES): m/z 188.0 [M+H]⁺, RT=0.34 min.

### Preparation 255: tert-butyl N-[(1S)-1-[(6-amino-5-bromo-2-pyridyl)carbamoyl]-2,2-dicyclopropyl-ethyl]carbamate

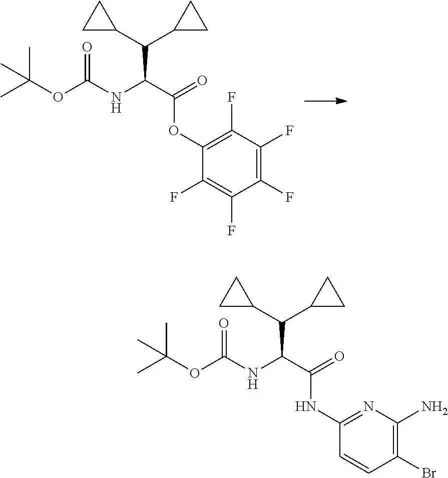 [SMILES: CC(C)(C)OC(=O)N[C@H](C(=O)Oc1c(F)c(F)c(F)c(F)c1F)C(C1CC1)C1CC1 ; CC(C)(C)OC(=O)N[C@H](C(=O)Nc1ccc(Br)c(N)n1)C(C1CC1)C1CC1]

According to the method of Preparation 240 the compound of Preparation 237 (145 mg, 0.333 mmol) was reacted with the compound of Preparation 254 (110 mg, 0.59 mmol). The crude mixture of regioisomers was purified by prep. basic HPLC to afford the title compound (44 mg, 19% yield). LCMS (METHOD 3) (ES): m/z 341.1 [(M-Boc)+H]+, RT=0.78 min.

### Preparation 256: tert-butyl N-[(1S)-1-[[6-amino-5-[3,5-dimethyl-1-(2-trimethylsilylethoxymethyl)pyrazol-4-yl]-2-pyridyl]carbamoyl]-2,2-dicyclopropyl-ethyl]carbamate

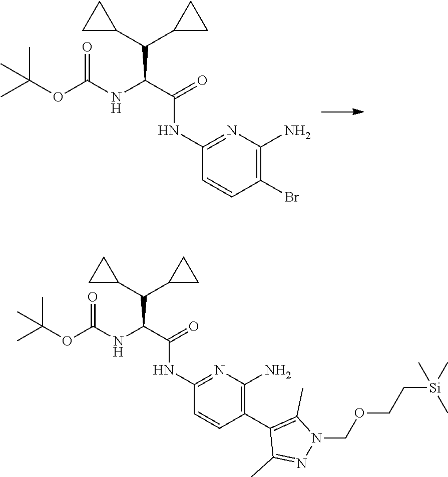 [SMILES: CC(C)(C)OC(=O)N[C@H](C(=O)Nc1ccc(Br)c(N)n1)C(C1CC1)C1CC1 ; Cc1nn(COCC[Si](C)(C)C)c(C)c1-c1ccc(NC(=O)[C@@H](NC(=O)OC(C)(C)C)C(C2CC2)C2CC2)nc1N]

According to the method of Preparation 7 the compound of Preparation 255 (44.0 mg, 0.064 mmol) was reacted with the compound of Preparation 41 (100 mg, 0.28 mmol). The crude material was purified by prep. basic HPLC to afford the title compound (27 mg, 72% yield). 1H NMR (400 MHz, CDCl₃) δ 8.19 (s, 1H), 7.59 (d, J=8.0 Hz, 1H), 7.26 (d, J=8.0 Hz, 1H), 5.37 (d, J=1.3 Hz, 3H), 4.42 (s, 1H), 4.28 (s, 2H), 3.63 (dd, J=8.6, 7.6 Hz, 2H), 2.19 (s, 3H), 2.12 (s, 3H), 1.47 (s, 9H), 0.99-0.85 (m, 3H), 0.75 (tt, J=8.7, 4.4 Hz, 2H), 0.51 (dq, J=26.7, 8.6 Hz, 4H), 0.37-0.19 (m, 4H), 0.00 (s, 9H); LCMS (METHOD 3) (ES): m/z 585.5 [M+H]⁺, RT=0.96 min.

### Preparation 257: (2S)-2-amino-N-[6-amino-5-(3,5-dimethyl-1H-pyrazol-4-yl)-2-pyridyl]-3,3-dicyclopropyl-propanamide hydrochloride

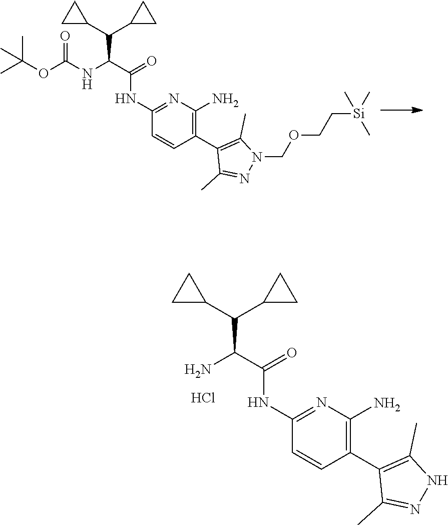 [SMILES: Cc1nn(COCC[Si](C)(C)C)c(C)c1-c1ccc(NC(=O)[C@@H](NC(=O)OC(C)(C)C)C(C2CC2)C2CC2)nc1N ; Cc1n[nH]c(C)c1-c1ccc(NC(=O)[C@@H](N)C(C2CC2)C2CC2)nc1N ; Cl]

Hydrogen chloride (4M soln in dioxane, 1.0 mL) was added to a solution of the compound of Preparation 256 (27.0 mg, 0.046 mmol) in DCM (2 mL) and MeOH (2 mL) and stirred at room temperature for 20 minutes. The reaction mixture was concentrated in vacuo to leave crude title compound as a colourless solid. (23 mg, assume 100% yield). Material used without characterization.

### Preparation 258: tert-butyl N-(5-bromo-6-fluoro-2-pyridyl)-N-tert-butoxycarbonyl-carbamate

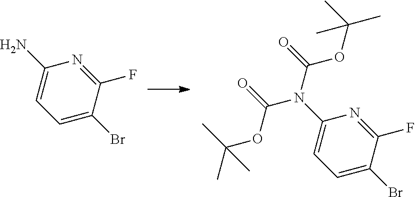 [SMILES: Nc1ccc(Br)c(F)n1 ; CC(C)(C)OC(=O)N(C(=O)OC(C)(C)C)c1ccc(Br)c(F)n1]

Triethylamine (5 mL) was added to a solution of 5-bromo-6-fluoro-pyridin-2-amine (1.90 g, 9.9 mmol), tert-butoxycarbonyl tert-butyl carbonate (6.5 g, 30 mmol) and DMAP (122 mg, 1.0 mmol) in DCM (20 mL) and stirred at room temperature for 24 hours. A further portion of tert-butoxycarbonyl tert-butyl carbonate (6.5 g, 30 mmol) was added and again the reaction mixture was stirred for 24 hours. The reaction mixture was concentrated in vacuo and the obtained crude compound was purified by silica column chromatography (230-400 mesh), eluting with EtOAc in heptane, to afford the title compound as a colourless solid. (3.10 g, 80% yield). 1H NMR (400 MHz, CDCl₃) δ 7.96 (t, J=8.4 Hz, 1H), 7.13 (dd, J=8.1, 1.0 Hz, 1H), 1.47 (s, 18H).

### Preparation 259: 4-bromo-6-fluoro-5-iodo-pyridin-2-amine

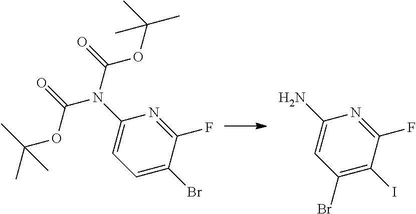 [SMILES: CC(C)(C)OC(=O)N(C(=O)OC(C)(C)C)c1ccc(Br)c(F)n1 ; Nc1cc(Br)c(I)c(F)n1]

n-Butyllithium (2.7 M soln in heptane, 3.6 mL, 9.80 mmol) was added dropwise to a solution of diisopropylamine (1.4 mL, 9.80 mmol) in THF (10 mL) at −75° C. The reaction mixture was stirred at −75° C. for 10 minutes. A solution of the compound of Preparation 258 (3.20 g, 6.5 mmol) in THF (10 mL) was added dropwise, maintaining the internal temperature at −75° C. On complete addition the reaction mixture was stirred at this temperature for 90 minutes. A solution of iodine (2.5 g, 9.8 mmol) in THF (20 mL) was added and the reaction mixture was stirred for 30 minutes at −75° C. The reaction mixture was warmed to −20° C. and quenched with H₂O (30 mL). The mixture was extracted with Et₂O (3×30 mL). The combined organic phase was dried over MgSO₄, filtered and concentrated in vacuo. The obtained crude compound was purified by silica column chromatography (230-400 mesh), eluting with EtOAc in heptane, to afford the intermediate tert-butyl N-(5-bromo-6-fluoro-4-iodo-2-pyridyl)-N-tert-butoxycarbonyl-carbamate as a yellow oil. (1.60 g, 38% yield); 1H NMR (400 MHz, CDCl₃) δ 7.61 (s, 1H), 1.50 (s, 18H). LCMS (METHOD 3) (ES): m/z 515.1 [M−H]⁻, RT=0.97 min. TFA (2.5 M, 0.96 mL) was added to a solution of intermediate tert-butyl N-(5-bromo-6-fluoro-4-iodo-2-pyridyl)-N-tert-butoxycarbonyl-carbamate (1.55 g, 2.40 mmol) in DCM (5 mL) and the reaction mixture was stirred at room temperature for 16 hours. The reaction mixture was concentrated in vacuo, dissolved in MeOH and purified directly by prep. basic HPLC to afford the title compound. (0.58 g, 76% yield). LCMS (METHOD 3) (ES): m/z 314.9 [M−H]⁻, RT=0.42 min.

### Preparation 260: tert-butyl N-[(1S)-1-[(4-bromo-6-fluoro-5-iodo-2-pyridyl)carbamoyl]-2,2-dicyclopropyl-ethyl]carbamate

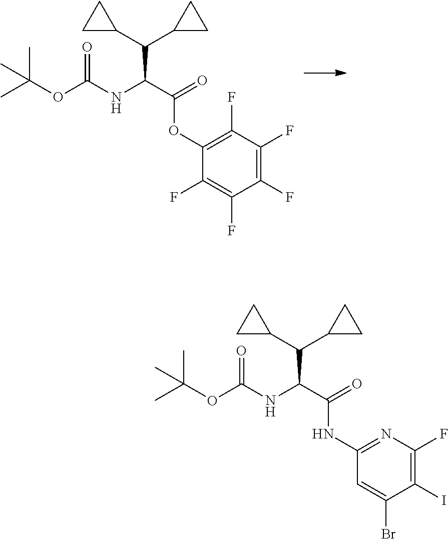 [SMILES: CC(C)(C)OC(=O)N[C@H](C(=O)Oc1c(F)c(F)c(F)c(F)c1F)C(C1CC1)C1CC1 ; CC(C)(C)OC(=O)N[C@H](C(=O)Nc1cc(Br)c(I)c(F)n1)C(C1CC1)C1CC1]

According to the method of Preparation 240 the compound of Preparation 237 (840 mg, 1.93 mmol) was reacted with the product of Preparation 259 (580 mg, 1.80 mmol). The crude mixture was purified by prep. basic HPLC to afford the title compound (530 mg, 51% yield). LCMS (METHOD 3) (ES): m/z 568.2 [M+H]⁺, RT=1.00 min.

### Preparation 261: tert-butyl N-[(1S)-1-[(4-amino-6-fluoro-5-iodo-2-pyridyl)carbamoyl]-2,2-dicyclopropyl-ethyl]carbamate

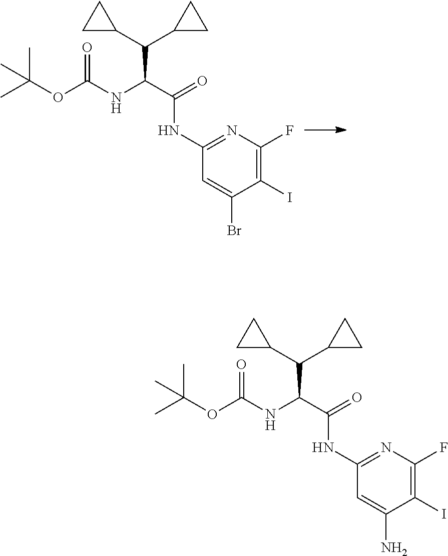 [SMILES: CC(C)(C)OC(=O)N[C@H](C(=O)Nc1cc(Br)c(I)c(F)n1)C(C1CC1)C1CC1 ; CC(C)(C)OC(=O)N[C@H](C(=O)Nc1cc(N)c(I)c(F)n1)C(C1CC1)C1CC1]

Sodium azide (70.0 mg, 1.08 mmol) was added to a mixture of the compound of Preparation 260 (255 mg, 0.45 mmol), N,N′-dimethylethane-1,2-diamine (25 mg, 0.28 mmol) and copper iodide (10 mg, 0.052 mmol) in EtOH (14 mL) and H₂O (6 mL). The solution was degassed, the reaction vial was sealed and heated at 95° C. for 2 days. The reaction mixture was concentrated in vacuo, dissolved in MeOH and purified by basic prep. HPLC to afford the title compound as a colourless solid (42 mg, 18% yield). 1H NMR (400 MHz, CDCl₃) δ 8.34 (s, 1H), 7.52 (s, 1H), 5.38 (s, 1H), 5.01 (s, 2H), 4.44 (s, 1H), 1.46 (s, 9H), 0.87 (td, J=9.1, 4.4 Hz, 1H), 0.80-0.61 (m, 2H), 0.61-0.34 (m, 4H), 0.23 (ddp, J=18.0, 9.3, 4.5 Hz, 4H); LCMS (METHOD 3) (ES): m/z 503.2 [M−H]⁻, RT=0.85 min.

### Preparation 262: tert-butyl N-[(1S)-1-[[4-amino-5-[3,5-dimethyl-1-(2-trimethylsilylethoxymethyl)pyrazol-4-yl]-6-fluoro-2-pyridyl]carbamoyl]-2,2-dicyclopropyl-ethyl]carbamate

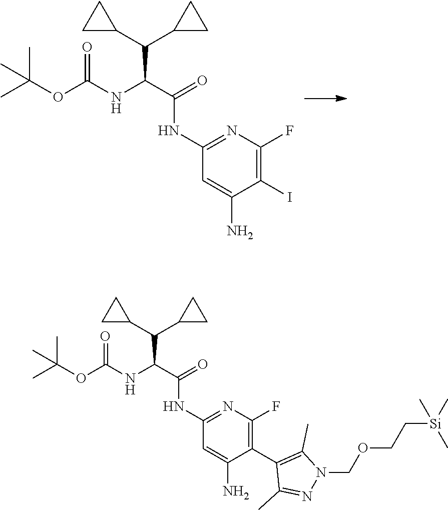 [SMILES: CC(C)(C)OC(=O)N[C@H](C(=O)Nc1cc(N)c(I)c(F)n1)C(C1CC1)C1CC1 ; Cc1nn(COCC[Si](C)(C)C)c(C)c1-c1c(N)cc(NC(=O)[C@@H](NC(=O)OC(C)(C)C)C(C2CC2)C2CC2)nc1F]

According to the method of Preparation 7 the compound of Preparation 261 (31.0 mg, 0.061 mmol) was reacted with the compound of Preparation 41 (50 mg, 0.14 mmol). The crude material was purified by prep. basic HPLC to afford the title compound (19 mg, 51% yield). LCMS (METHOD 3) (ES): m/z 603.6 [M+H]⁺, RT=0.96 min.

### Preparation 263: (2S)-2-amino-N-[4-amino-5-[3,5-dimethyl-1-(2-trimethylsilylethoxymethyl)pyrazol-4-yl]-6-fluoro-2-pyridyl]-3,3-dicyclopropyl-propanamide

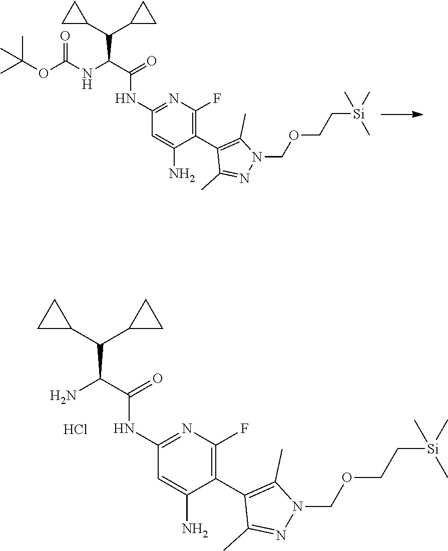 [SMILES: Cc1nn(COCC[Si](C)(C)C)c(C)c1-c1c(N)cc(NC(=O)[C@@H](NC(=O)OC(C)(C)C)C(C2CC2)C2CC2)nc1F ; Cc1nn(COCC[Si](C)(C)C)c(C)c1-c1c(N)cc(NC(=O)[C@@H](N)C(C2CC2)C2CC2)nc1F ; Cl]

Hydrogen chloride (4M soln in dioxane, 1.0 mL) was added to a solution of the compound of Preparation 262 (24.0 mg, 0.04 mmol) in DCM (2 mL) and stirred at room temperature for 1 hour. The product precipitated, so the liquid was decanted and the solid was dried to leave crude title compound (22 mg, assume 100% yield). LCMS (METHOD 3) (ES): m/z 501.3 [M−H]⁻, RT=0.84 min.

### Preparation 264: N-[(1S)-1-[[4-amino-5-[3,5-dimethyl-1-(2-trimethylsilylethoxymethyl)pyrazol-4-yl]-6-fluoro-2-pyridyl]carbamoyl]-2,2-dicyclopropyl-ethyl]-2-ethyl-pyrazole-3-carboxamide

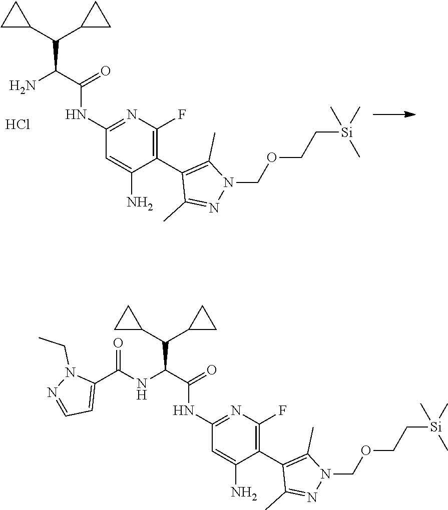

According to the method of Preparation 11 the compound of Preparation 263 (22 mg, 0.04 mmol) was reacted with 2-ethylpyrazole-3-carboxylic acid (12.0 mg, 0.085 mmol) to afford the title compound after prep. basic HPLC (24.0 mg, assume 100% yield). LCMS (METHOD 3) (ES): m/z 623.6 [M−H]⁻, RT=0.90 min.

### Preparation 265: 2-[(2,5-dibromo-3-pyridyl)oxymethoxy]ethyl-trimethyl-silane

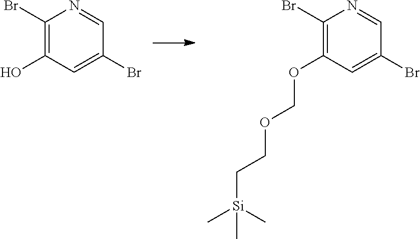 [SMILES: Oc1cc(Br)cnc1Br ; C[Si](C)(C)CCOCOc1cc(Br)cnc1Br]

SEM chloride (0.70 mL, 3.95 mmol) was added to a solution of 2,5-dibromopyridin-3-ol (500 mg, 1.98 mmol) and triethylamine (0.55 mL, 3.95 mmol) in DCM (7.5 mL) at 0° C. The reaction mixture was stirred at room temperature for 72 hours. The reaction mixture was concentrated in vacuo and the residue was dissolved in EtOAc (30 mL). The organic layer was washed with H₂O (3×10 mL), saturated brine solution (10 mL), dried over Na₂SO₄, filtered and concentrated in vacuo. The obtained crude compound was purified by silica column chromatography (230-400 mesh), eluting with EtOAc in heptane, to afford the title compound as a colourless oil. (677 mg, 89% yield). 1H NMR (600 MHz, CDCl₃) δ 8.11 (d, J=2.1 Hz, 1H), 7.59 (d, J=2.1 Hz, 1H), 5.32 (s, 2H), 3.89-3.72 (m, 2H), 1.03-0.90 (m, 2H), 0.02 (s, 9H).

### Preparation 266: 2-[[2-bromo-5-[3,5-dimethyl-1-(2-trimethylsilylethoxymethyl)pyrazol-4-yl]-3-pyridyl]oxymethoxy]ethyl-trimethyl-silane (266a) and 2-[[5-bromo-2-[3,5-dimethyl-1-(2-trimethylsilylethoxymethyl)pyrazol-4-yl]-3-pyridyl]oxymethoxy]ethyl-trimethyl-silane (266b)

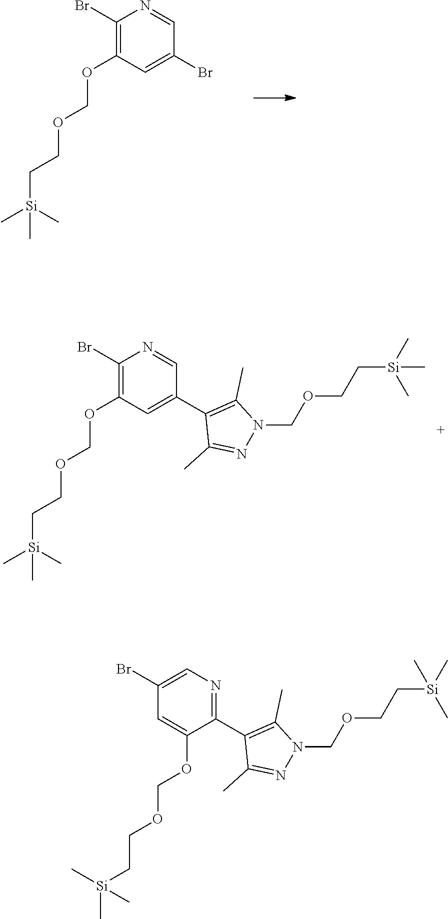

According to the method of Preparation 7 the compound of Preparation 265 (200 mg, 0.52 mmol) was reacted with the compound of Preparation 41 (184 mg, 0.52 mmol). The obtained crude compound was purified by silica column chromatography (230-400 mesh), eluting with EtOAc in heptane, to afford the title compounds:

(266a): (40.8 mg, 15% yield); 1H NMR (600 MHz, CDCl₃) δ 7.94 (d, J=2.0 Hz, 1H), 7.36 (d, J=2.0 Hz, 1H), 5.39 (s, 2H), 5.34 (s, 2H), 3.88-3.76 (m, 2H), 3.66-3.57 (m, 2H), 2.32 (s, 3H), 2.25 (s, 3H), 1.03-0.81 (m, 4H), 0.00 (s, 9H), −0.01 (s, 9H); LCMS (METHOD 3) (ES): m/z 530.3 [M+H]⁺, RT=1.12 min.

(266b): (50.8 mg, 18% yield); 1H NMR (600 MHz, CDCl₃) δ 8.40 (d, J=1.9 Hz, 1H), 7.75 (d, J=2.0 Hz, 1H), 5.39 (s, 2H), 5.18 (s, 2H), 3.77-3.67 (m, 2H), 3.67-3.56 (m, 2H), 2.26 (s, 3H), 2.19 (s, 3H), 1.03-0.86 (m, 4H), 0.00 (s, 9H), −0.01 (s, 9H); LCMS (METHOD 3) (ES): m/z 530.3 [M+H]⁺, RT=1.14 min.

### Preparation 267: tert-butyl N-[(1S)-1-(dicyclopropylmethyl)-2-[[5-[3,5-dimethyl-1-(2-trimethylsilylethoxymethyl)pyrazol-4-yl]-3-(2-trimethylsilylethoxymethoxy)-2-pyridyl]amino]-2-oxo-ethyl]carbamate

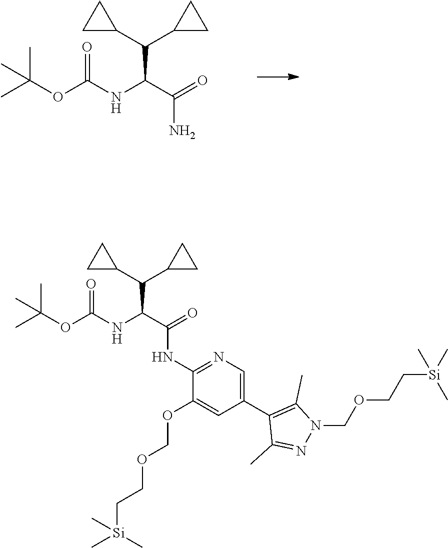 [SMILES: CC(C)(C)OC(=O)N[C@H](C(N)=O)C(C1CC1)C1CC1 ; Cc1nn(COCC[Si](C)(C)C)c(C)c1-c1cnc(NC(=O)[C@@H](NC(=O)OC(C)(C)C)C(C2CC2)C2CC2)c(OCOCC[Si](C)(C)C)c1]

According to the method of Preparation 90 the compound of Preparation 89 (26 mg, 0.097 mmol) was reacted with the compound of Preparation 266a (54 mg, 0.102 mmol) to afford the title compound after flash chromatography (27.0 mg, 39% yield). LCMS (METHOD 3) (ES): m/z 716.7 [M+H]⁺, RT=1.12 min.

### Preparation 268: (2S)-2-amino-3,3-dicyclopropyl-N-[5-[3,5-dimethyl-1-(2-trimethylsilylethoxymethyl)pyrazol-4-yl]-3-hydroxy-2-pyridyl]propenamide

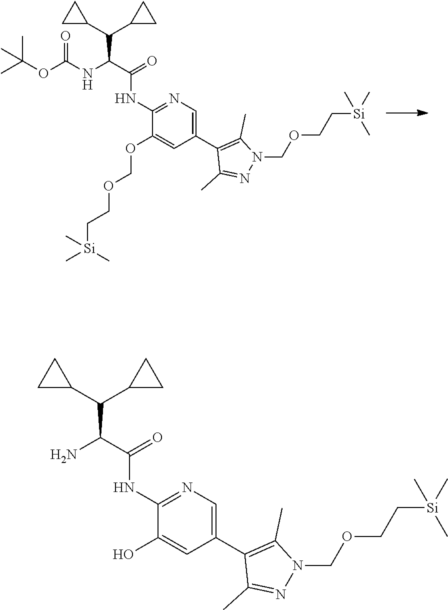 [SMILES: Cc1nn(COCC[Si](C)(C)C)c(C)c1-c1cnc(NC(=O)[C@@H](NC(=O)OC(C)(C)C)C(C2CC2)C2CC2)c(OCOCC[Si](C)(C)C)c1 ; Cc1nn(COCC[Si](C)(C)C)c(C)c1-c1cnc(NC(=O)[C@@H](N)C(C2CC2)C2CC2)c(O)c1]

Hydrogen chloride (4M soln in dioxane, 1.0 mL) was added to a solution of the compound of Preparation 267 (30.0 mg, 0.042 mmol) in MeOH (2 mL) and stirred at room temperature for 1.5 hours. The reaction mixture was diluted with MeOH (4 mL), then concentrated in vacuo to leave crude title compound (21 mg, assume 100% yield). LCMS (METHOD 3) (ES): m/z 486.4 [M+H]⁺, RT=0.78 min.

### Preparation 269: N-[(1S)-1-(dicyclopropylmethyl)-2-[[5-[3,5-dimethyl-1-(2-trimethylsilylethoxymethyl) pyrazol-4-yl]-3-hydroxy-2-pyridyl]amino]-2-oxo-ethyl]-2-ethyl-pyrazole-3-carboxamide

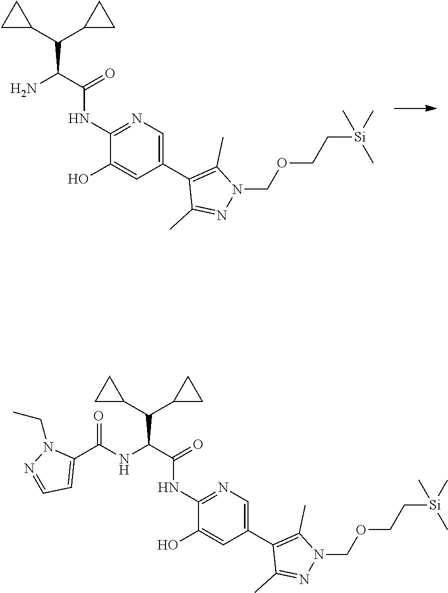

According to the method of Preparation 11 the compound of Preparation 268 (21 mg, 0.04 mmol) was reacted with 2-ethylpyrazole-3-carboxylic acid (5.9 mg, 0.04 mmol) to afford the title compound after prep. acidic HPLC (5.0 mg, 20% yield). LCMS (METHOD 3) (ES): m/z 608.5 [M+H]⁺, RT=0.95 min.

### Preparation 270: N-[(1S)-1-(dicyclopropylmethyl)-2-[[5-[3,5-dimethyl-1-(2-trimethylsilylethoxymethyl)pyrazol-4-yl]-3-hydroxy-2-pyridyl]amino]-2-oxo-ethyl]-2-isopropyl-pyrazole-3-carboxamide

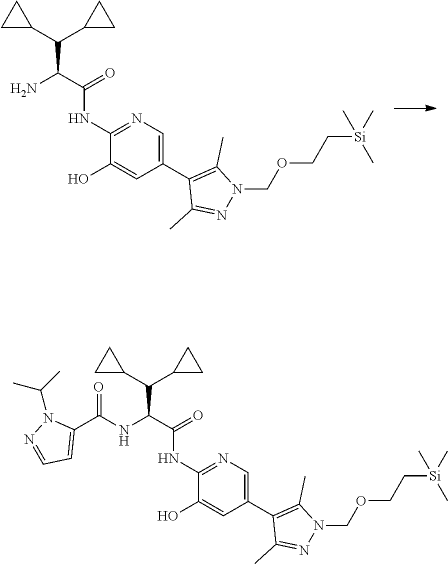

According to the method of Preparation 11 the compound of Preparation 268 (21 mg, 0.04 mmol) was reacted with 2-isopropylpyrazole-3-carboxylic acid (6.5 mg, 0.04 mmol) to afford the title compound after prep. acidic HPLC (2.0 mg, 7.7% yield). LCMS (METHOD 3) (ES): m/z 622.5 [M+H]⁺, RT=0.98 min.

### Preparation 271: tert-butyl N-[(1S)-1-(dicyclopropylmethyl)-2-[[6-[3,5-dimethyl-1-(2-trimethylsilylethoxymethyl)pyrazol-4-yl]-5-(2-trimethylsilylethoxymethoxy)-3-pyridyl]amino]-2-oxo-ethyl]carbamate

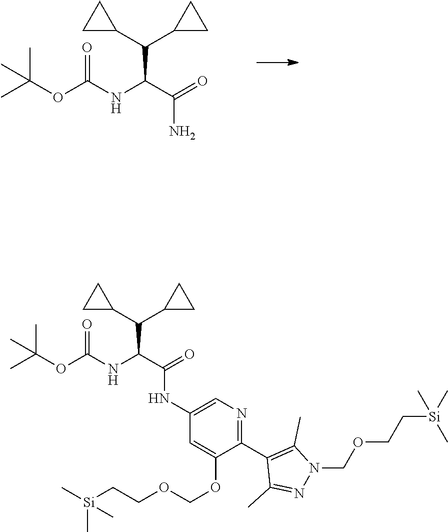 [SMILES: CC(C)(C)OC(=O)N[C@H](C(N)=O)C(C1CC1)C1CC1 ; Cc1nn(COCC[Si](C)(C)C)c(C)c1-c1ncc(NC(=O)[C@@H](NC(=O)OC(C)(C)C)C(C2CC2)C2CC2)cc1OCOCC[Si](C)(C)C]

According to the method of Preparation 90 the compound of Preparation 89 (25 mg, 0.093 mmol) was reacted with the compound of Preparation 266b (51 mg, 0.098 mmol) to afford the title compound after flash chromatography (32.0 mg, 48% yield). 1H NMR (400 MHz, CDCl₃) δ 8.37 (d, J=2.2 Hz, 1H), 8.21 (s, 1H), 8.06 (d, J=2.2 Hz, 1H), 5.39 (s, 3H), 5.19 (s, 2H), 4.42 (dd, J=8.3, 4.6 Hz, 1H), 3.75-3.56 (m, 4H), 2.27 (s, 3H), 2.20 (s, 3H), 1.50 (s, 9H), 1.04-0.87 (m, 5H), 0.87-0.71 (m, 2H), 0.67-0.41 (m, 4H), 0.41-0.18 (m, 4H), 0.00 (s, 9H), −0.01 8s, 9H); LCMS (METHOD 3) (ES): m/z 716.7 [M+H]⁺, RT=1.10 min.

### Preparation 272: (2S)-2-amino-3,3-dicyclopropyl-N-[6-[3,5-dimethyl-1-(2-trimethylsilylethoxymethyl)pyrazol-4-yl]-5-hydroxy-3-pyridyl]propenamide

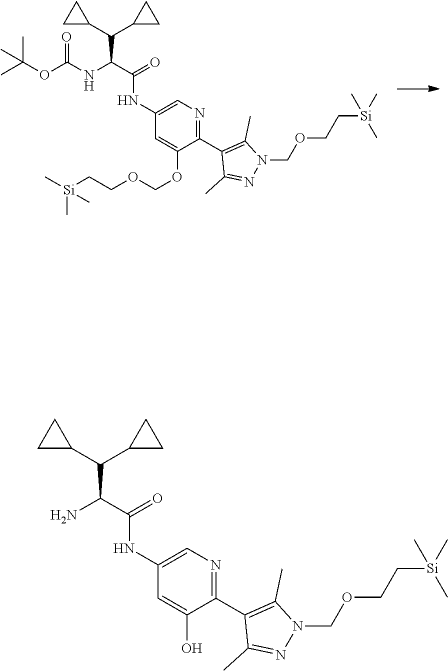 [SMILES: Cc1nn(COCC[Si](C)(C)C)c(C)c1-c1ncc(NC(=O)[C@@H](NC(=O)OC(C)(C)C)C(C2CC2)C2CC2)cc1OCOCC[Si](C)(C)C ; Cc1nn(COCC[Si](C)(C)C)c(C)c1-c1ncc(NC(=O)[C@@H](N)C(C2CC2)C2CC2)cc1O]

Hydrogen chloride (4M soln in dioxane, 1.0 mL) was added to a solution of the compound of Preparation 271 (14.0 mg, 0.02 mmol) in MeOH (2 mL) and stirred at room temperature for 1.5 hours. The reaction mixture was diluted with MeOH (4 mL), then concentrated in vacuo to leave crude title compound (10 mg, assume 100% yield). LCMS (METHOD 3) (ES): m/z 486.4 [M+H]⁺, RT=0.77 min.

### Preparation 273: N-[(1S)-1-(dicyclopropylmethyl)-2-[[6-[3,5-dimethyl-1-(2-trimethylsilylethoxymethyl)pyrazol-4-yl]-5-hydroxy-3-pyridyl]amino]-2-oxo-ethyl]-2-ethyl-pyrazole-3-carboxamide

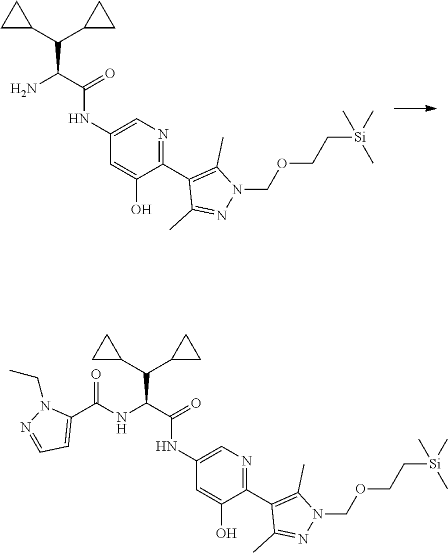

According to the method of Preparation 11 the compound of Preparation 272 (10 mg, 0.02 mmol) was reacted with 2-ethylpyrazole-3-carboxylic acid (2.7 mg, 0.02 mmol) to afford the title compound after prep. acidic HPLC (5.0 mg, 42% yield). LCMS (METHOD 3) (ES): m/z 608.5 [M+H]⁺, RT=0.81 min.

### Preparation 274: N-[(1S)-1-(dicyclopropylmethyl)-2-[[6-[3,5-dimethyl-1-(2-trimethylsilylethoxymethyl)pyrazol-4-yl]-5-hydroxy-3-pyridyl]amino]-2-oxo-ethyl]-2-isopropyl-pyrazole-3-carboxamide

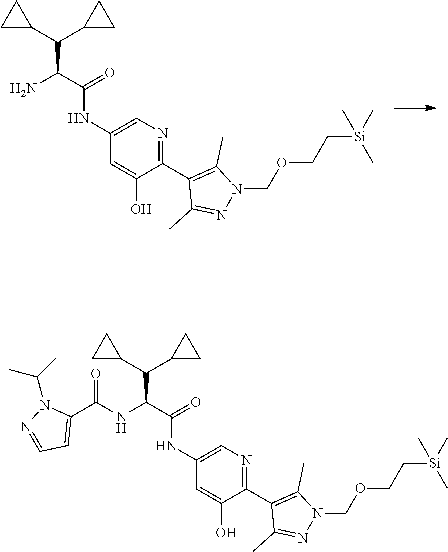

According to the method of Preparation 11 the compound of Preparation 272 (10 mg, 0.02 mmol) was reacted with 2-isopropylpyrazole-3-carboxylic acid (3.0 mg, 0.02 mmol) to afford the title compound after prep. acidic HPLC (5.0 mg, 41% yield). LCMS (METHOD 3) (ES): m/z 622.5 [M+H]⁺, RT=0.85 min.

### Preparation 275: N-[(1S)-1-[[6-chloro-5-[3,5-dimethyl-1-(2-trimethylsilylethoxymethyl)pyrazol-4-yl]-2-pyridyl]carbamoyl]-2,2-dicyclopropyl-ethyl]-3-isopropyl-triazole-4-carboxamide

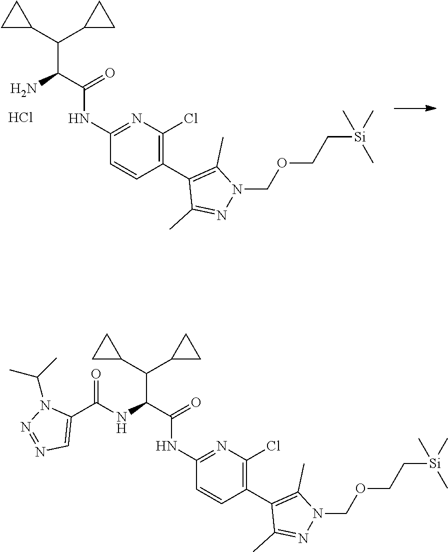

According to the method of Preparation 11 the compound of Preparation 140 (26 mg, 0.049 mmol) was reacted with 3-isopropyltriazole-4-carboxylic acid (7.6 mg, 0.049 mmol) to afford the title compound after prep. acidic HPLC (22 mg, 70% yield). LCMS (METHOD 3) (ES): m/z 641.5 [M+H]⁺, RT=0.98 min.

### Preparation 276: 3-methyl-1-tetrahydropyran-2-yl-pyrazole

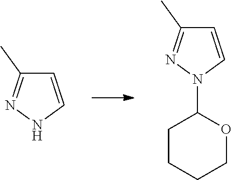

TFA (1 mL) was added to a solution of 3-methyl-1H-pyrazole (8.4 g, 102 mmol) and 3,4-dihydro-2H-pyran (10.3 g, 123 mmol) in toluene (25 mL) and the reaction mixture was stirred at 90° C. for 18 hours. K₂CO₃ was added portionwise until the solution was basic. The mixture was filtered and the filtrate was concentrated in vacuo to afford the title compound as an unseparable mixture of regioisomers. (16.9 g, 99% yield, 4:1 regioisomer mix); GCMS (ES): m/z 166.1 [M+H]⁺, RT=8.65 and 8.74 min.

### Preparation 277: 3-methyl-5-(1,1,2,2,2-pentadeuterioethyl)-1-tetrahydropyran-2-yl-pyrazole

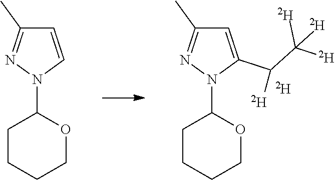

n-Butyllithium (9.6 mL, 24.0 mmol) was added slowly to a solution of the compound mix of Preparation 276 (5.0 g, 24.0 mmol) in THF (20 mL) at −65° C. The reaction mixture was stirred for 30 minutes then 1,1,1,2,2-pentadeuterio-2-iodo-ethane (4.6 g, 29.0 mmol) was added. The resulting reaction mixture was stirred at −65° C. for 1 hour, then at room temperature for 1 hour. The reaction mixture was concentrated in vacuo. The residue was dissolved in Et₂O (25 mL), washed with saturated brine solution (10 mL), dried over Na₂SO₄, filtered and concentrated in vacuo to leave the title compound as an orange oil. (5.55 g, 93% yield); GCMS (ES): m/z 199.1 [M+H]⁺, RT=9.69 min.

### Preparation 278: 4-bromo-3-methyl-5-(1,1,2,2,2-pentadeuterioethyl)-1-tetrahydropyran-2-yl-pyrazole

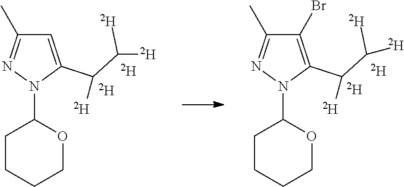

NBS (4.71 g, 26.5 mmol) was added to a solution of the compound of Preparation 277 (5.55 g, 27.8 mmol) in MeCN at room temperature. The reaction mixture was stirred for 1 hour then concentrated in vacuo. The residue was dissolved in H₂O (350 mL) and extracted with Et₂O (3×80 mL). The combined organic phase was dried over Na₂SO₄, filtered and concentrated in vacuo to afford the title compound as an orange oil. (7.24 g, 93% yield); GCMS (ES): m/z 277.0 [M+H]⁺, RT=11.08 min.

### Preparation 279: 3-methyl-5-(1,1,2,2,2-pentadeuterioethyl)-1-tetrahydropyran-2-yl-4-(4,4,5,5-tetramethyl-1,3,2-dioxaborolan-2-yl)pyrazole

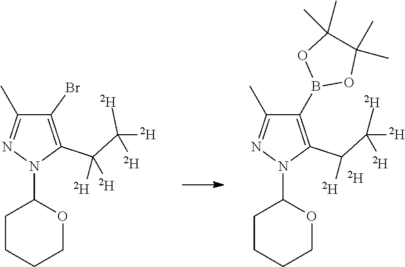

n-Butyllithium (2.5 M, 15.0 mL, 36.4 mmol) was added dropwise to a solution of the compound of Preparation 278 (7.24 g, 26.0 mmol) in THF (80 mL) at −75° C. The reaction mixture was stirred for 15 minutes, then 2-isopropoxy-4,4,5,5-tetramethyl-1,3,2-dioxaborolane (5.84 mL, 28.6 mmol) was added. The reaction mixture was stirred to room temperature over 1 hour. The mixture was quenched with saturated aq. NH₄Cl (60 mL) and extracted with Et₂O (3×50 mL). The combined organic phase was dried over Na₂SO₄, filtered and concentrated in vacuo. The obtained crude compound was purified by silica column chromatography (230-400 mesh), eluting with EtOAc in heptane, to afford the title compound as a colourless oil. (5.46 g, 64% yield); 1H NMR (400 MHz, CDCl₃) δ 5.17 (dd, J=10.3, 2.5 Hz, 1H), 4.07 (ddt, J=11.6, 4.3, 2.1 Hz, 1H), 3.62 (td, J=11.5, 2.4 Hz, 1H), 2.49 (tdd, J=12.4, 10.3, 4.1 Hz, 1H), 2.35 (s, 3H), 2.15-2.01 (m, 1H), 1.91-1.81 (m, 1H), 1.81-1.50 (m, 4H), 1.28 (s, 12H).

### Preparation 280: tert-butyl N-[(1S)-1-(dicyclopropylmethyl)-2-[[6-fluoro-5-[3-methyl-5-(1,1,2,2,2-pentadeuterioethyl)-1-tetrahydropyran-2-yl-pyrazol-4-yl]-2-pyridyl]amino]-2-oxo-ethyl]carbamate

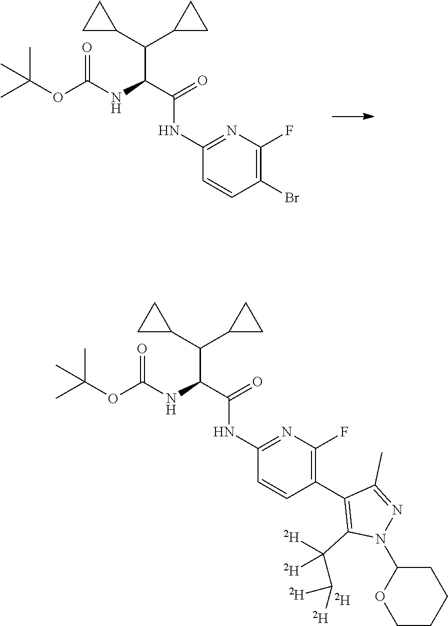

According to the method of Preparation 22, the compound of Preparation 90 (0.89 g, 2.0 mmol) was reacted with the compound of Preparation 279 (0.87 g, 2.70 mmol) to afford the title compound after flash chromatography, as a colourless solid (0.95 g, 85% yield). 1H NMR (400 MHz, CDCl₃) δ 8.48 (s, 1H), 8.14 (dd, J=8.1, 1.5 Hz, 1H), 7.64 (dd, J=9.6, 8.0 Hz, 1H), 5.33 (s, 1H), 5.22 (dd, J=10.4, 2.4 Hz, 1H), 4.45 (s, 1H), 4.19-4.05 (m, 1H), 3.66 (td, J=11.6, 2.4 Hz, 1H), 2.64-2.45 (m, 1H), 2.16 (s, 3H), 2.12 (d, J=12.5 Hz, 1H), 1.94 (dd, J=13.1, 3.1 Hz, 1H), 1.84-1.64 (m, 2H), 1.58 (d, J=13.2 Hz, 1H), 1.48 (s, 9H), 1.01-0.91 (m, 1H), 0.73 (dqd, J=16.6, 8.4, 4.4 Hz, 2H), 0.63-0.39 (m, 4H), 0.26 (ddt, J=18.4, 9.4, 4.9 Hz, 4H). LCMS (METHOD 3) (ES): m/z 561.6 [M+H]⁺, RT=0.95 min.

### Preparation 281: (2S)-2-amino-3,3-dicyclopropyl-N-[6-fluoro-5-[3-methyl-5-(1,1,2,2,2-pentadeuterioethyl)-1H-pyrazol-4-yl]-2-pyridyl]propenamide hydrochloride

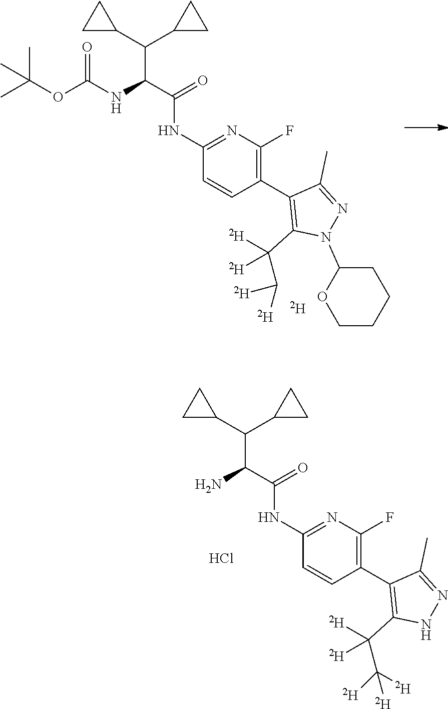

Hydrogen chloride (3M soln in 1,4-dioxane, 10.0 mL) was added to a solution of the compound of Preparation 280 (950 mg, 1.70 mmol) in MeOH (20 mL) and stirred at 50° C. for 1.5 hours. The reaction mixture was diluted with MeOH (4 mL), then concentrated in vacuo to leave crude title compound (700 mg, assume 100% yield). LCMS (METHOD 3) (ES): m/z 377.4 [M+H]⁺, RT=0.67 min.

### Preparation 282: ethyl (2S)-2-(4-methoxyanilino)-2-[(1S)-5-methylene-2-oxo-cyclohexyl]acetate

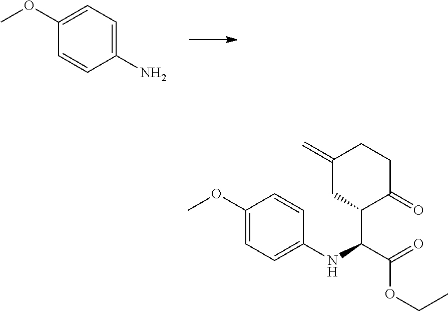

Ethyl 2-oxoacetate (4.97 g, 24.4 mmol) was added to a suspension of 4-methoxyaniline (3.0 g, 24.4 mmol) and MgSO₄ (5.0 g, 41.5 mmol) in toluene (30 mL) at room temperature and stirred for 30 minutes. The reaction mixture was filtered, washing the cake with toluene (30 mL). The filtrate was concentrated in vacuo to leave intermediate ethyl-2-(4-methoxyphenyl)iminoacetate (5.05 g 100% yield). (2S)-pyrrolidine-2-carboxylic acid (600 mg, 5.21 mmol) was added to a solution of the intermediate ethyl-2-(4-methoxyphenyl)iminoacetate (5.05 g, 24.4 mmol) and 4-methylenecyclohexanone (6.0 g, 49.0 mmol) in DMSO (30 mL) and stirred at room temperature for 3 hours. The reaction mixture was poured into TBME/H₂O (100 mL, 1:1) and the phases were separated. The aqueous phase was washed with TBME (2×50 mL). The combined organic phase was dried over Na₂SO₄, filtered and concentrated in vacuo. The obtained crude compound was purified by silica column chromatography (230-400 mesh), eluting with EtOAc in heptane, to afford the title compound as a tan oil. (5.60 g, 72% yield). 1H NMR (600 MHz, CDCl₃) δ 6.81-6.69 (m, 4H), 4.95 (dt, J=5.7, 1.4 Hz, 2H), 4.28 (d, J=5.4 Hz, 1H), 4.23-4.08 (m, 3H), 3.74 (s, 3H), 2.92-2.82 (m, 1H), 2.73-2.40 (m, 6H), 1.22 (t, J=7.1 Hz, 3H); LCMS (METHOD 3) (ES): m/z 318.1 [M+H]⁺, RT=0.79 min.

### Preparation 283: ethyl (2S)-2-(4-methoxyanilino)-2-[(1S)-3-methylenecyclohexyl]acetate

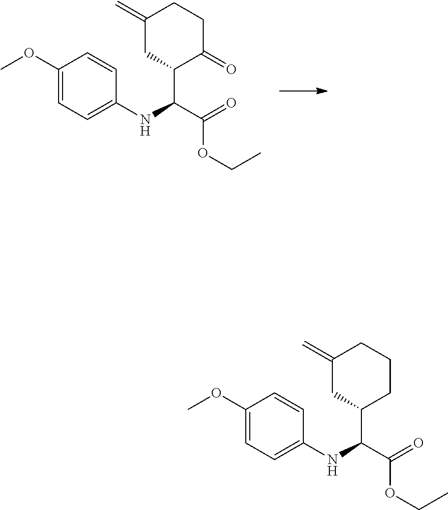 [SMILES: C=C1CCC(=O)[C@H]([C@H](Nc2ccc(OC)cc2)C(=O)OCC)C1 ; C=C1CCC[C@H]([C@H](Nc2ccc(OC)cc2)C(=O)OCC)C1]

4-Methylbenzenesulfonohydrazide (2.60 g, 14.0 mmol) was added to a solution of the compound of Preparation 282 (3.50 g, 11.0 mmol) and the reaction mixture was stirred at 70° C. for 2 hours. The reaction mixture was concentrated in vacuo then taken up in TBME. The excess 4-methylbenzenesulfonohydrazide was filtered off and the filtrate was concentrated in vacuo to give intermediate ethyl (2S)-2-(4-methoxyanilino)-2-[(1S,2E)-5-methylene-2-(p-tolylsulfonylhydrazono)cyclohexyl]acetate (5.2 g, 97% yield); (ES): m/z 484.3 [M−H]⁻, RT=0.84 and 0.91 min (E/Z isomers). NaBH₄ (0.6 g, 20 mmol) was added portion wise to a solution of ethyl (2S)-2-(4-methoxyanilino)-2-[(1S,2E)-5-methylene-2-(p-tolylsulfonylhydrazono)cyclohexyl]acetate (3.7 g, 7.6 mmol) in AcOH (25 mL) and THF (10 mL) at 5° C. over 30 minutes. The reaction mixture was quenched with H₂O (100 mL) and the precipitate was collected by filtration. The solid was washed with H₂O, then dissolved in DCM, dried over MgSO₄, filtered and concentrated in vacuo to give intermediate ethyl (2S)-2-(4-methoxyanilino)-2-[(1S)-5-methylene-2-[2-(p-tolylsulfonyl)hydrazino]cyclohexyl]acetate (3.7 g, assume 100 yield %). Sodium acetate trihydrate (3.7 g, 27 mmol) was added to a solution of ethyl (2S)-2-(4-methoxyanilino)-2-[(1S,2E)-5-methylene-2-(p-tolylsulfonylhydrazno)cyclohexyl]acetate (3.7 g, 7.6 mmol) in EtOH (30 mL) and stirred at 100° C. for 1 hour. The cooled reaction mixture was diluted with TBME (60 mL) and the precipitate was removed via filtration. The filtrate was dried over Na₂SO₄, filtered and concentrated in vacuo. The obtained crude compound was purified by silica column chromatography (230-400 mesh), eluting with EtOAc in heptane, to afford the title compound as a tan oil. (1.16 g, 35% yield). 1H NMR (600 MHz, CDCl₃) δ 6.80-6.71 (m, 2H), 6.68-6.56 (m, 2H), 4.73-4.60 (m, 2H), 4.24-4.08 (m, 2H), 3.90-3.78 (m, 2H), 3.73 (d, J=1.9 Hz, 3H), 2.43 (ddt, J=13.1, 3.7, 1.6 Hz, 1H), 2.26 (ddd, J=13.0, 5.1, 3.3 Hz, 1H), 2.02-1.92 (m, 2H), 1.85 (dpd, J=14.0, 6.6, 3.6 Hz, 2H), 1.81-1.71 (m, 1H), 1.44-1.31 (m, 2H), 1.26-1.18 (m, 3H).

### Preparation 284: ethyl (2S)-2-(benzyloxycarbonylamino)-2-[(1S)-3-methylenecyclohexyl]acetate

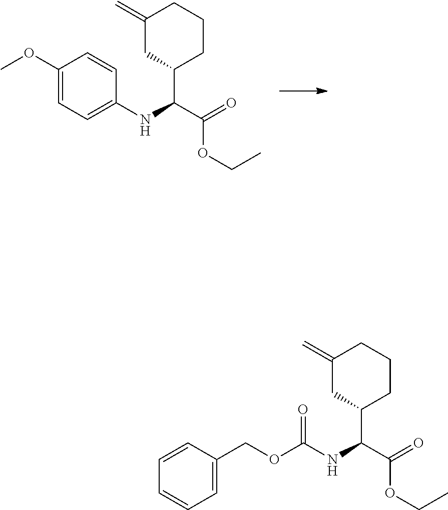 [SMILES: C=C1CCC[C@H]([C@H](Nc2ccc(OC)cc2)C(=O)OCC)C1 ; C=C1CCC[C@H]([C@H](NC(=O)OCc2ccccc2)C(=O)OCC)C1]

CAN (8.2 g, 15.0 mmol) was added to a solution of the compound of Preparation 283 (1.3 g, 4.3 mmol) in MeCN (30 mL) and H₂O (30 mL) and stirred at room temperature for 1 hour. The reaction mixture was basified with solid K₂CO₃ to pH 8. Benzyl carbonochloridate (1.2 mL, 8.4 mmol) was added and the reaction mixture was stirred at room temperature for 2 hours. The reaction mixture was filtered though Celite, washing with TBME (150 mL). The organic phase was separated, washed with Na₂S₂O₃·5H₂O (0.4 M, 50 mL), dried over MgSO₄, filtered and concentrated in vacuo. The obtained crude compound was purified by silica column chromatography (230-400 mesh), eluting with EtOAc in heptane, to afford the title compound as a red oil. (1.16 g, 82% yield). 1H NMR (400 MHz, CDCl₃) δ 7.43-7.29 (m, 5H), 5.38-5.25 (m, 1H), 5.11 (s, 2H), 4.63 (dd, J=15.0, 2.1 Hz, 2H), 4.33 (dd, J=9.2, 4.2 Hz, 1H), 4.21 (qt, J=7.9, 3.8 Hz, 2H), 2.21 (dd, J=34.5, 12.0 Hz, 2H), 1.88 (q, J=12.0, 11.2 Hz, 4H), 1.76 (d, J=12.4 Hz, 1H), 1.28 (q, J=10.4, 8.7 Hz, 5H).

### Preparation 285: ethyl (2S)-2-(benzyloxycarbonylamino)-2-[(7S)-spiro[2.5]octan-7-yl]acetate

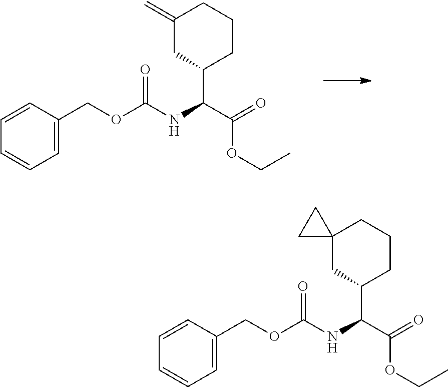 [SMILES: C=C1CCC[C@H]([C@H](NC(=O)OCc2ccccc2)C(=O)OCC)C1 ; CCOC(=O)[C@@H](NC(=O)OCc1ccccc1)[C@H]1CCCC2(CC2)C1]

Diethylzinc (15% w/w solution in hexane, 2.1 mL, 2.3 mmol) was added to a solution of the compound of Preparation 284 (323 mg, 0.975 mmol) and chloroiodomethane (0.4 mL, 5.0 mmol) in DCM (10 mL) at 5° C. The reaction mixture was stirred at 5° C. for 20 minutes then for 2 hours at room temperature. The reaction mixture was quenched with aqueous hydrogen chloride (1M, 10 mL) and extracted with DCM (2×20 mL). The organic phase was dried over MgSO₄, filtered and concentrated in vacuo. The obtained crude compound was purified by silica column chromatography (230-400 mesh), eluting with EtOAc in heptane, to afford the title compound as a colourless oil. (182 mg, 54% yield). 1H NMR (600 MHz, CDCl₃) δ 7.39-7.28 (m, 5H), 5.35-5.20 (m, 1H), 5.10 (s, 2H), 4.28 (dd, J=9.1, 5.3 Hz, 1H), 4.25-4.03 (m, 2H), 2.02-1.81 (m, 1H), 1.78-1.69 (m, 2H), 1.67-1.52 (m, 2H), 1.42 (qq, J=11.9, 4.2 Hz, 1H), 1.26 (t, J=7.1 Hz, 3H), 1.12 (qd, J=12.7, 3.8 Hz, 1H), 0.79 (dq, J=13.2, 2.2 Hz, 1H), 0.75-0.66 (m, 1H), 0.32-0.22 (m, 2H), 0.22-0.07 (m, 2H).

### Preparation 286: ethyl (2S)-2-[(2-ethylpyrazole-3-carbonyl)amino]-2-[(7S)-spiro[2.5]octan-7-yl]acetate

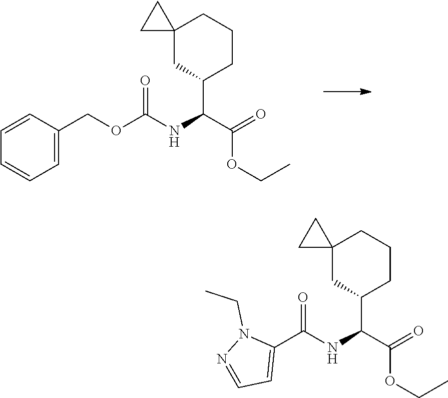

Triethylsilane (0.5 mL, 3.13 mmol) was added to a mixture of the compound of Preparation 285 (160 mg, 0.46 mmol) and Pd/C (10%, 30 mg) in MeOH and the reaction mixture was stirred at room temperature for 1 hour. The mixture was filtered through Celite washing with MeOH (50 mL). The filtrate was concentrated in vacuo to leave intermediate ethyl (2S)-2-amino-2-[(7S)-spiro[2.5]octan-7-yl]acetate (98 mg, assume 100% yield). According to the method of Preparation 11 the intermediate ethyl (2S)-2-amino-2-[(7S)-spiro[2.5]octan-7-yl]acetate (98 mg, 0.46 mmol) was reacted with 2-ethylpyrazole-3-carboxylic acid (77.9 mg, 0.56 mmol) to give the title compound as an off-white solid (126 mg, 81% yield). 1H NMR (600 MHz, CDCl₃) δ 7.47 (d, J=2.0 Hz, 1H), 6.56 (d, J=2.1 Hz, 1H), 6.44 (d, J=8.7 Hz, 1H), 4.68 (dd, J=8.7, 5.1 Hz, 1H), 4.64-4.49 (m, 2H), 4.23 (ddq, J=40.3, 10.8, 7.1 Hz, 2H), 2.13-2.03 (m, 1H), 1.81 (d, J=3.8 Hz, 1H), 1.75 (dt, J=13.0, 3.4 Hz, 1H), 1.63 (tt, J=12.8, 2.5 Hz, 2H), 1.43 (t, J=7.2 Hz, 4H), 1.30 (t, J=7.1 Hz, 3H), 1.12 (qd, J=12.8, 3.7 Hz, 1H), 0.82 (d, J=13.4 Hz, 1H), 0.75 (ddt, J=13.0, 3.9, 2.1 Hz, 1H), 0.35-0.24 (m, 2H), 0.22 (dddd, J=9.2, 5.4, 3.9, 1.7 Hz, 1H), 0.15 (dddd, J=9.3, 5.7, 3.9, 1.7 Hz, 1H); LCMS (METHOD 3) (ES): m/z 334.2 [M+H]⁺, RT=0.85 min.

### Preparation 287: N-[(1S)-2-[[5-[3,5-dimethyl-1-(2-trimethylsilylethoxymethyl)pyrazol-4-yl]-6-fluoro-2-pyridyl]amino]-2-oxo-1-[(7S)-spiro[2.5]octan-7-yl]ethyl]-2-ethyl-pyrazole-3-carboxamide

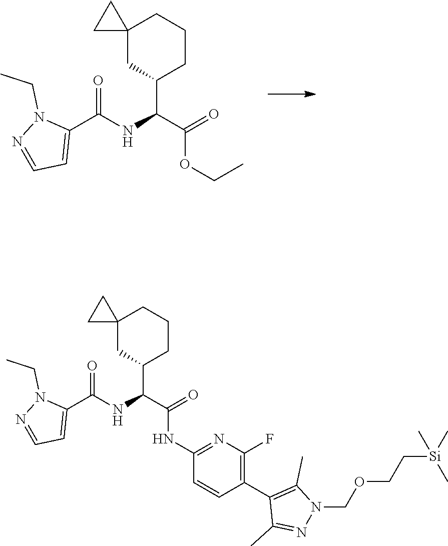 [SMILES: CCOC(=O)[C@@H](NC(=O)c1ccnn1CC)[C@H]1CCCC2(CC2)C1 ; CCn1nccc1C(=O)N[C@H](C(=O)Nc1ccc(-c2c(C)nn(COCC[Si](C)(C)C)c2C)c(F)n1)[C@H]1CCCC2(CC2)C1]

According to the method of Preparation 27 the compound of Preparation 286 (50.0 mg, 0.15 mmol) was reacted with the product from Preparation 41 (53.0 mg, 0.16 mmol) to afford the title compound after prep. acidic HPLC, as a colourless solid (32 mg, 34% yield). LCMS (METHOD 3) (ES): m/z 624.4 [M+H]⁺, RT=1.01 min.

### Preparation 288: 2-ethyl-N-[(1S)-2-[[5-[5-ethyl-3-methyl-1-(2-trimethylsilylethoxymethyl)pyrazol-4-yl]-6-fluoro-2-pyridyl]amino]-2-oxo-1-[(7S)-spiro[2.5]octan-7-yl]ethyl]pyrazole-3-carboxamide

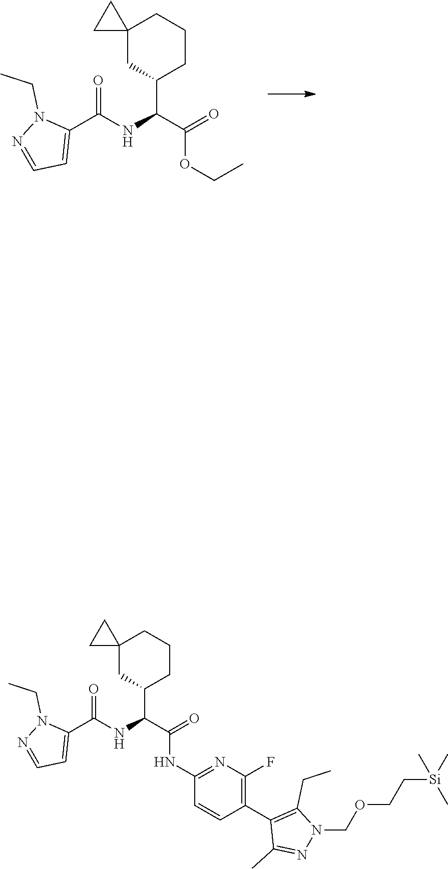 [SMILES: CCOC(=O)[C@@H](NC(=O)c1ccnn1CC)[C@H]1CCCC2(CC2)C1 ; CCc1c(-c2ccc(NC(=O)[C@@H](NC(=O)c3ccnn3CC)[C@H]3CCCC4(CC4)C3)nc2F)c(C)nn1COCC[Si](C)(C)C]

According to the method of Preparation 27 the compound of Preparation 286 (70.0 mg, 0.21 mmol) was reacted with the product from Preparation 39 (77.3 mg, 0.22 mmol) to afford the title compound after prep. acidic HPLC, as a colourless solid (48 mg, 36% yield). LCMS (METHOD 3) (ES): m/z 638.4 [M+H]⁺, RT=1.04 min.

### Preparation 289: (4,5,6,7-tetrachloro-1,3-dioxo-isoindolin-2-yl)spiro[2.3]hexane-5-carboxylate

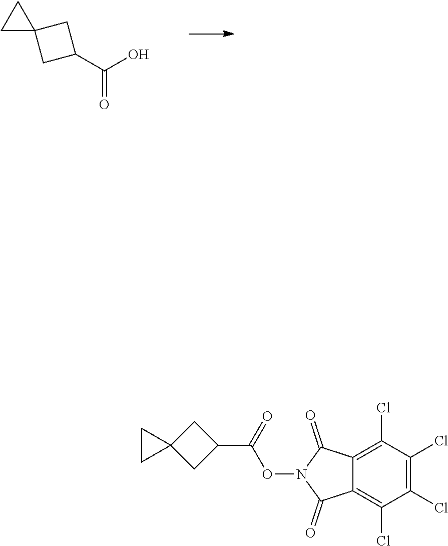 [SMILES: O=C(O)C1CC2(CC2)C1 ; O=C(ON1C(=O)c2c(Cl)c(Cl)c(Cl)c(Cl)c2C1=O)C1CC2(CC2)C1]

A dry round-bottomed flask was charged with spiro[2.3]hexane-5-carboxylic acid (2.2 g, 17.4 mmol), N-hydroxy-tetrachlorophthalimide (5.76 g, 1.1 eq.), and DMAP (0.44 g, 0.2 eq.). DCM was added (20 mL), and the mixture was stirred vigorously under a N₂ atmosphere. N,N′-Diisopropylcarbodiimide (2.94 mL, 19.2 mmol) was then added dropwise via syringe, and the mixture was allowed to stir at room temperature until the acid was consumed (monitored by TLC). The mixture was filtered through a Celite pad, rinsed with additional DCM and concentrated in vacuo. The crude product was purified by silica gel (100-200 mesh) column chromatography (1% EtOAc in pet. ether as eluent) to afford the title compound as a white solid (2.6 g, 36%). 1H NMR (400 MHz, CDCl₃) δ 3.68-3.60 (m, 1H), 2.70-2.65 (t, J=20 Hz, 2H), 2.49-2.44 (t, J=20 Hz, 2H), 0.55-0.51 (t, J=16 Hz, 4H).

### Preparation 290: ethyl (2S)-2-spiro[2.3]hexan-5-yl-2-[(2,4,6-trimethylphenyl)sulfinylamino]acetate

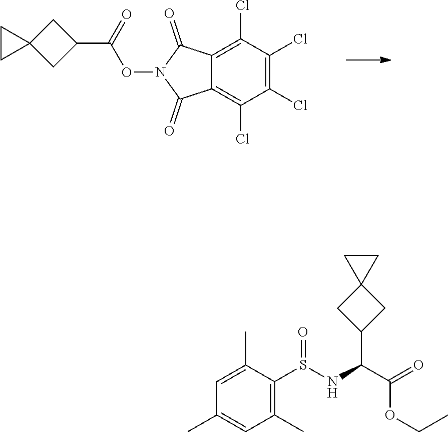 [SMILES: O=C(ON1C(=O)c2c(Cl)c(Cl)c(Cl)c(Cl)c2C1=O)C1CC2(CC2)C1 ; CCOC(=O)[C@@H](NS(=O)c1c(C)cc(C)cc1C)C1CC2(CC2)C1]

A culture tube was charged with the compound of Preparation 289 (2.6 g, 6.35 mmol), ethyl (S) (E)-2-((2,4,6-trimethylphenyl)sulfinylimino)acetate (Synthesised according to Angew. Chem. Int. Ed. 2018, 57, 14560) (1.70 g, 6.35 mmol), Ni(OAc)₂·4H₂O (0.39 g, 1.59 mmol) and zinc dust (1.20 g, 19.1 mmol). The tube was then evacuated and backfilled with argon (three times). Anhydrous NMP (20 mL) was added using a syringe. The mixture was stirred overnight at room temperature. Then, the reaction mixture was diluted with Et₂O and water and filtered through a Celite pad, and then extracted with Et₂O (2×30 mL) washed with water, brine and dried over Na₂SO₄. After filtration, the organic layer was concentrated in vacuo (water bath at 30° C.), and the residue was purified by silica gel (100-200 mesh) column chromatography (EtOAc in pet. ether as eluent) to afford the title compound as a colourless oil (1.0 g, 45%). 1H NMR (400 MHz, CDCl₃) δ 6.87-6.84 (s, J=12 Hz, 2H), 5.08-5.05 (d, J=12 Hz, 1H), 4.22-4.13 (m, 2H), 3.99-3.95 (t, J=16 Hz, 1H), 2.87 (s, 3H), 2.71 (m, 1H), 2.59 (s, 6H), 2.07-2.02 (m, 4H), 1.28-1.24 (t, J=16 Hz, 3H), 0.42-0.34 (t, J=32 Hz, 4H); LCMS (METHOD 2) (ESI): m/z 350.32 [M+H]⁺; RT=2.82 min (ACQUITY BEH C18 column, 0.05% FA in water with MeCN).

### Preparation 291: ethyl (2S)-2-amino-2-spiro[2.3]hexan-5-yl-acetate

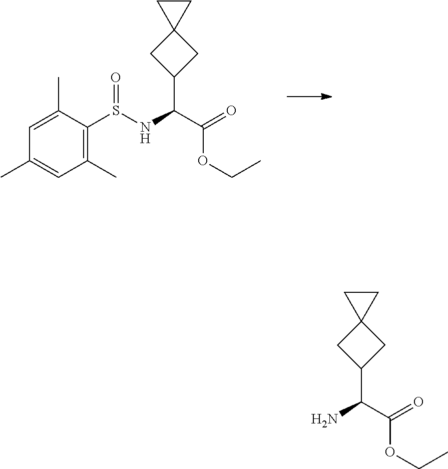 [SMILES: CCOC(=O)[C@@H](NS(=O)c1c(C)cc(C)cc1C)C1CC2(CC2)C1 ; CCOC(=O)[C@@H](N)C1CC2(CC2)C1]

To a stirred solution of the compound of Preparation 290 (1.0 g, 2.86 mmol) in MeOH (10 mL) at 0° C. under N₂ was added 4M hydrogen chloride in MeOH (2 mL). The reaction was stirred at room temperature for 4 hours. The reaction mixture was concentrated in vacuo to afford the title compound (0.35 g, 67% yield) as a colourless oil which was used in the next step without further purification.

### Preparation 292: ethyl (2S)-2-(tert-butoxycarbonylamino)-2-spiro[2.3]hexan-5-yl-acetate

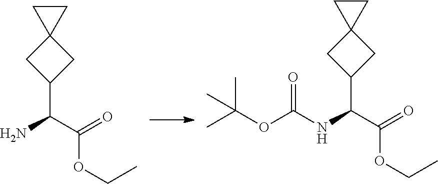 [SMILES: CCOC(=O)[C@@H](N)C1CC2(CC2)C1 ; CCOC(=O)[C@@H](NC(=O)OC(C)(C)C)C1CC2(CC2)C1]

To a stirred solution of the compound of Preparation 291 (350 mg, 1.91 mmol) in DCM (10 mL) was added triethylamine (0.59 mL, 4.44 mmol) and Boc₂O (420 mg, 1.91 mmol) at 0° C. under N₂. The reaction mixture was stirred at room temperature for 6 hours. The reaction was diluted with ice-cold water (10 mL) and extracted with DCM (2×30 mL). The combined organic layers were dried over Na₂SO₄ and concentrated in vacuo. The crude residue was purified by silica gel (100-200 mesh) column chromatography (EtOAc in pet. ether as eluent) to afford the title compound (420 mg, 77% yield) as a pale yellow gum. which was used directly in the next step. 1H NMR (400 MHz, DMSO-d6) δ 7.21-7.19 (d, J=8 Hz, 1H), 4.2-4.1 (m, 3H), 2.6 (m, 1H), 2.1-1.9 (m, 4H), 1.201 (m, 3H), 1.3 (s, 9H) 0.501-0.302 (m, 4H).

### Preparation 293: (2S)-2-(tert-butoxycarbonylamino)-2-spiro[2.3]hexan-5-yl-acetic acid

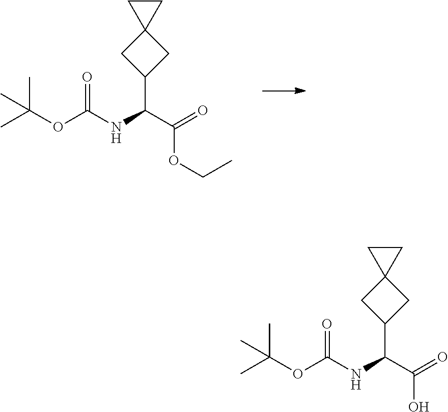 [SMILES: CCOC(=O)[C@@H](NC(=O)OC(C)(C)C)C1CC2(CC2)C1 ; CC(C)(C)OC(=O)N[C@H](C(=O)O)C1CC2(CC2)C1]

According to the method of Preparation 148, the compound of Preparation 292 (420 mg, 1.48 mmol) was reacted to afford the crude title compound (300 mg, 79% yield). 1H NMR (400 MHz, DMSO-d6) δ 7.05-7.038 (d, J=4.8 Hz, 1H), 4.020 (s, 1H), 3.945-3.923 (t, J=8.8 Hz, 1H), 2.670-2.607 (m, 1H), 1.5-1.3 (m, 13H), 0.401-0.340 (t, J=24.4 Hz, 4H); LCMS (METHOD 2) (ESI): m/z 254.24 [M−H]⁻; RT=2.11 (ACQUITY BEH C18 column, 0.1% FA in water with MeCN).

### Preparation 294: methyl (2S)-2-(tert-butoxycarbonylamino)-2-spiro[2.3]hexan-5-yl-acetate

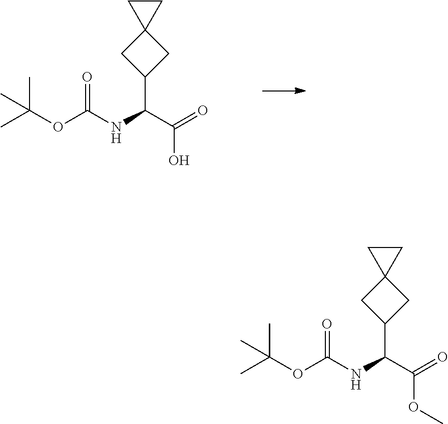 [SMILES: CC(C)(C)OC(=O)N[C@H](C(=O)O)C1CC2(CC2)C1 ; COC(=O)[C@@H](NC(=O)OC(C)(C)C)C1CC2(CC2)C1]

Methyl iodide (0.036 mL, 0.59 mmol) was added to a mixture of the compound of Preparation 293 (100 mg, 0.39 mmol) and K₂CO₃ (162 mg, 1.17 mmol) in DMF (1 mL) at 0° C. The reaction mixture was stirred at room temperature for 3 hours, poured into ice water (10 mL) and extracted with EtOAc (2×30 mL). The combined organic layers were dried over Na₂SO₄ and concentrated in vacuo, to afford the crude title compound as a brown oil. (85 mg, 81% yield). Material used without further purification. GCMS: m/z: 269; 66%; RT=7.79 min (Method: D:\MassHunter\GCMS\1\methods\GVK01.M; Method Information: DB-5MS (30m×0.25 mm×0.25 μm); He=5.0 ml/min, Inj=230° C., Split=50:1, I.V=1.0 μL; Detector Temperature: 300° C., Programme: 100° C./1 min, 20*C/min/300*C/6.0 min).

### Preparation 295: methyl (2S)-2-amino-2-spiro[2.3]hexan-5-yl-acetate; hydrochloride

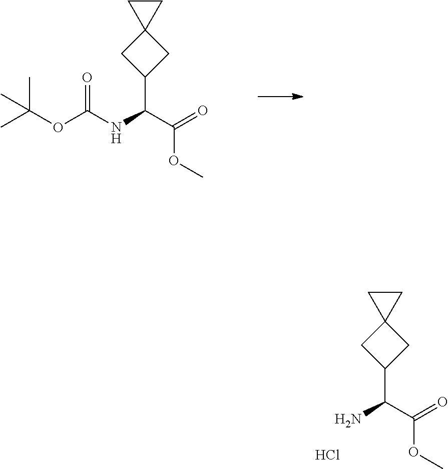 [SMILES: COC(=O)[C@@H](NC(=O)OC(C)(C)C)C1CC2(CC2)C1 ; Cl ; COC(=O)[C@@H](N)C1CC2(CC2)C1]

Hydrogen chloride (4M soln in dioxane, 0.8 mL) was added to a solution of the compound of Preparation 294 (85.0 mg, 0.31 mmol) in 1,4-dioxane (0.8 mL) and stirred at room temperature for 3 hours. The reaction mixture concentrated in vacuo to leave crude title compound as a brown oil. (80 mg, assume 100% yield). LCMS (METHOD 2) (ESI): m/z: 170 [M+H]⁺; 85%; RT=0.39 min (ACQUITY BEH C18 column, 0.05% FA in water with MeCN).

### Preparation 296: methyl (2S)-2-[(2-ethylpyrazole-3-carbonyl)amino]-2-spiro[2.3]hexan-5-yl-acetate

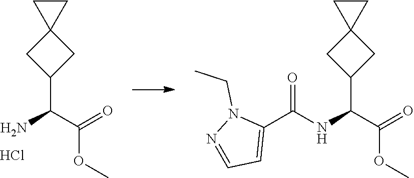 [SMILES: Cl ; COC(=O)[C@@H](N)C1CC2(CC2)C1 ; CCn1nccc1C(=O)N[C@H](C(=O)OC)C1CC2(CC2)C1]

According to the method of Preparation 11 the compound of Preparation 295 (80 mg, 0.31 mmol) was reacted with 2-ethylpyrazole-3-carboxylic acid (54 mg, 0.39 mmol) to afford the title compound after prep. TLC (EtOAc in pet. Ether) (50 mg, 37% yield). GCMS: m/z: 291; 67%; RT=7.79 min (Method: D:\MassHunter\GCMS\1\methods\GVK01.M; Method Information: DB-5MS (30m×0.25 mm×0.25 μm); He=5.0 ml/min, Inj=230° C., Split=50:1, I.V=1.0 μL; Detector Temperature: 300° C., Programme: 100° C./1 min, 20*C/min/300*C/6.0 min).

Preparation 297: N-[(1S)-2-[[5-[3,5-dimethyl-1-(2-trimethylsilylethoxymethyl)pyrazol-4-yl]-6-fluoro-2-pyridyl]amino]-2-oxo-1-spiro[2.3]hexan-5-yl-ethyl]-2-ethyl-pyrazole-3-carboxamide

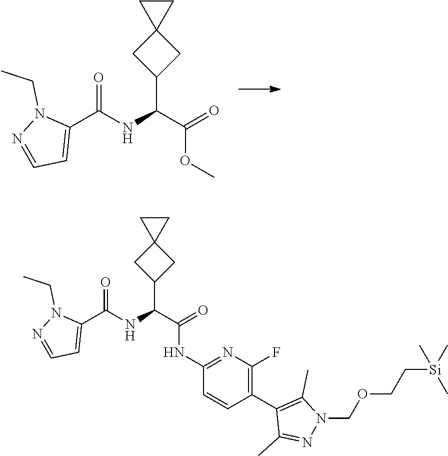 [SMILES: CCn1nccc1C(=O)N[C@H](C(=O)OC)C1CC2(CC2)C1 ; CCn1nccc1C(=O)N[C@H](C(=O)Nc1ccc(-c2c(C)nn(COCC[Si](C)(C)C)c2C)c(F)n1)C1CC2(CC2)C1]

According to the method of Preparation 27 the compound of Preparation 296 (50 mg, 0.17 mmol) was reacted with the compound of Preparation 41 (57 mg, 0.17 mmol) to afford the title compound as an off-white solid (80 mg, 49% yield). LCMS (METHOD 2) (ESI): m/z: 596 [M+H]⁺; 53%; RT=2.92 min (ACQUITY BEH C18 column, 0.05% FA in water with MeCN).

### Preparation 298: ethyl (2S)-2-amino-2-(4-methylcyclohexyl)acetate

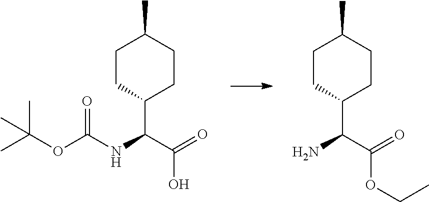 [SMILES: CC(C)(C)OC(=O)N[C@H](C(=O)O)[C@H]1CC[C@H](C)CC1 ; CCOC(=O)[C@@H](N)[C@H]1CC[C@H](C)CC1]

Thionyl chloride (3.9 mL, 53.7 mmol) was added dropwise to a solution of (S)-2-((tert-butoxycarbonyl)amino)-2-((1r,4S)-4-methylcyclohexyl)acetic acid (synthesis described in WO2018229079, 650 mg, 2.35 mmol) in EtOH (20 mL) at 0° C. After 1 hour the reaction mixture temperature was raised to 90° C. and the mixture was stirred for 16 hours. The cooled reaction mixture was basified to pH 9 with saturated aq. NaHCO₃ solution and extracted with EtOAc (2×100 mL). The combined organic layers were washed with saturated brine solution (30 mL), dried over Na₂SO₄ and concentrated in vacuo to afford the title compound as a gum (300 mg, 64% yield). 1H NMR (300 MHz, DMSO-d6) δ 4.115-4.034 (m, 2H), 3.07 (d, J=5.4 Hz, 1H), 1.677-1.593 (m, 5H), 1.525-1.401 (m, 2H), 1.24-1.001 (m, 6H), 0.903-0.826 (m, 5H); LCMS (METHOD 2) (ESI): m/z 200 [M+H]⁺; 58%; RT=4.09 min; (ACQUITY BEH C18 column, 5 mM ammonium bicarbonate in water with MeCN).

### Preparation 299: ethyl (2S)-2-(4-methylcyclohexyl)-2-[(3-methylisoxazole-4-carbonyl)amino]acetate

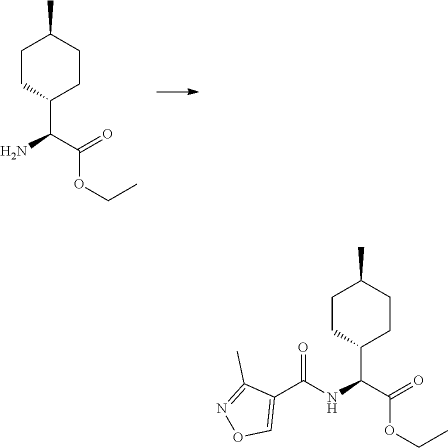 [SMILES: CCOC(=O)[C@@H](N)[C@H]1CC[C@H](C)CC1 ; CCOC(=O)[C@@H](NC(=O)c1conc1C)[C@H]1CC[C@H](C)CC1]

According to the method of Preparation 11 the compound of Preparation 298 (200 mg, 1.0 mmol) was reacted with 3-methylisoxazole-4-carboxylic acid (140 mg, 1.10 mmol) to afford the title compound after flash chromatography (280 mg, 90% yield). 1H NMR (300 MHz, DMSO-d6) δ 9.43-9.38 (m, 1H), 8.49 (d, J=8.1 Hz, 1H), 4.25 (t, J=7.5 Hz, 1H), 4.18-4.07 (m, 2H), 2.35 (s, 3H), 1.69 (br dd, J=3.5, 10.8 Hz, 5H), 1.22 (s, 3H), 1.17-1.04 (m, 3H), 0.94-0.81 (m, 5H); LCMS (ESI): ELSD (ESI): m/z: 309 [M+H]⁺; 89%; RT=2.19 min (ACQUITY BEH C18 column, 0.05% FA in water with MeCN).

### Preparation 300: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-3-methyl-isoxazole-4-carboxamide

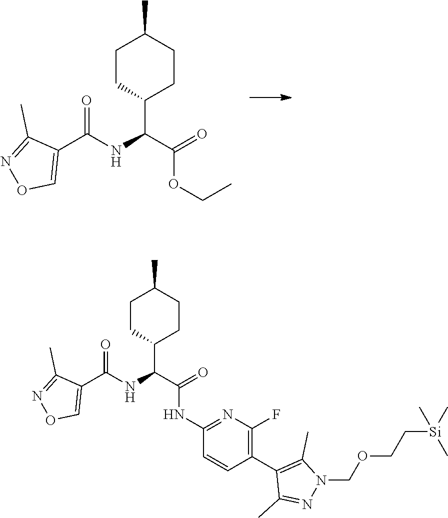 [SMILES: CCOC(=O)[C@@H](NC(=O)c1conc1C)[C@H]1CC[C@H](C)CC1 ; Cc1nocc1C(=O)N[C@H](C(=O)Nc1ccc(-c2c(C)nn(COCC[Si](C)(C)C)c2C)c(F)n1)[C@H]1CC[C@H](C)CC1]

According to the method of Preparation 27 the compound of Preparation 299 (140 mg, 0.45 mmol) was reacted with the compound of Preparation 41 (152 mg, 0.45 mmol) to afford the title compound as a yellow oil (100 mg, 37% yield). 1H NMR (300 MHz, DMSO-d6) δ 10.94 (s, 1H), 9.47 (s, 1H), 8.47 (br d, J=7.7 Hz, 1H), 8.17-8.04 (m, 1H), 7.93-7.83 (m, 1H), 5.39 (s, 2H), 4.55 (br t, J=7.7 Hz, 1H), 3.60 (br t, J=7.9 Hz, 2H), 2.40 (s, 3H), 2.22 (s, 3H), 2.17-2.02 (m, 5H), 1.92-1.55 (m, 3H), 1.41-1.23 (m, 5H), 0.99-0.81 (m, 5H), 0.06-0.05 (m, 9H); LCMS (METHOD 2) (ESI): m/z: 599 [M+H]⁺; 60%; RT=2.85 min (ACQUITY BEH C18 column, 0.05% FA in water with MeCN).

### Preparation 301: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-3-methyl-isoxazole-4-carboxamide

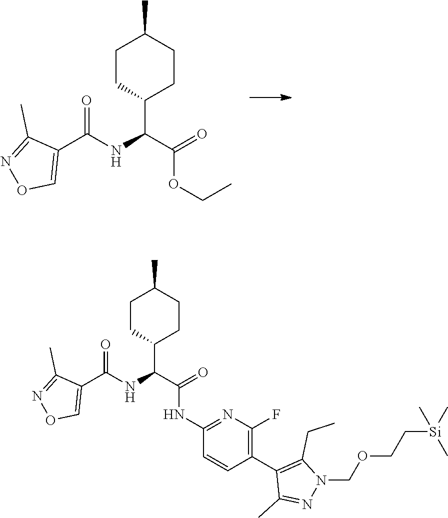 [SMILES: CCOC(=O)[C@@H](NC(=O)c1conc1C)[C@H]1CC[C@H](C)CC1 ; CCc1c(-c2ccc(NC(=O)[C@@H](NC(=O)c3conc3C)[C@H]3CC[C@H](C)CC3)nc2F)c(C)nn1COCC[Si](C)(C)C]

According to the method of Preparation 27 the compound of Preparation 299 (140 mg, 0.45 mmol) was reacted with the compound of Preparation 39 (159 mg, 0.45 mmol) to afford the title compound as a yellow oil (150 mg, 53% yield). LCMS (METHOD 2) (ESI): m/z: 613 [M+H]⁺; 55%; RT=2.60 min (ACQUITY BEH C18 column, 0.05% FA in water with MeCN).

### Preparation 302: ethyl (2S)-2-[(3-ethylisoxazole-4-carbonyl)amino]-2-(4-methylcyclohexyl)acetate

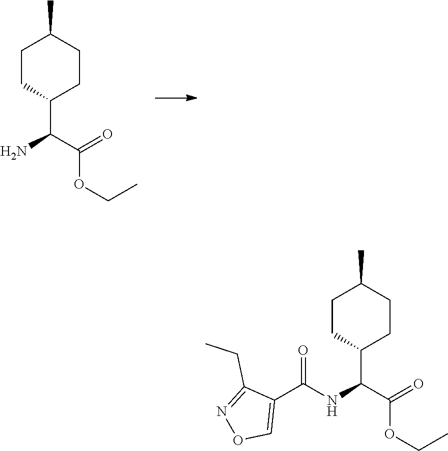 [SMILES: CCOC(=O)[C@@H](N)[C@H]1CC[C@H](C)CC1 ; CCOC(=O)[C@@H](NC(=O)c1conc1CC)[C@H]1CC[C@H](C)CC1]

According to the method of Preparation 11 the compound of Preparation 298 (130 mg, 0.65 mmol) was reacted with 3-ethylisoxazole-4-carboxylic acid (101 mg, 0.72 mmol) to afford the title compound as a colourless solid after flash chromatography (200 mg, 95% yield). LCMS (METHOD 2) (ESI): m/z: 323 [M+H]⁺; 97%; RT=2.55 min (ACQUITY BEH C18 column, 0.05% FA in water with MeCN).

### Preparation 303: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-3-ethyl-isoxazole-4-carboxamide

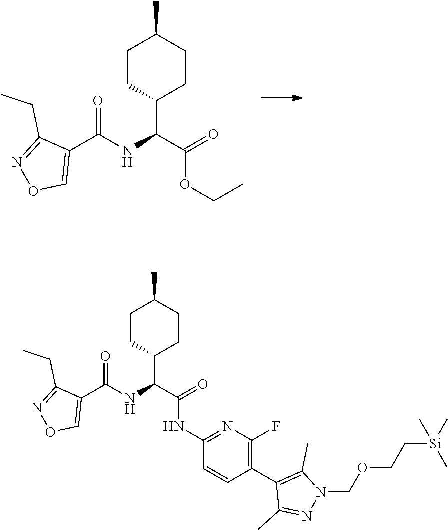 [SMILES: CCOC(=O)[C@@H](NC(=O)c1conc1CC)[C@H]1CC[C@H](C)CC1 ; CCc1nocc1C(=O)N[C@H](C(=O)Nc1ccc(-c2c(C)nn(COCC[Si](C)(C)C)c2C)c(F)n1)[C@H]1CC[C@H](C)CC1]

According to the method of Preparation 27 the compound of Preparation 302 (200 mg, 0.62 mmol) was reacted with the compound of Preparation 41 (208 mg, 0.62 mmol) to afford the title compound as a yellow oil (300 mg, 78% yield). LCMS (METHOD 2) (ESI): m/z: 613 [M+H]⁺; 39%; RT=3.99 min (ACQUITY BEH C18 column, 0.05% TFA in water with MeCN).

### Preparation 304: 3-ethyl-N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]isoxazole-4-carboxamide

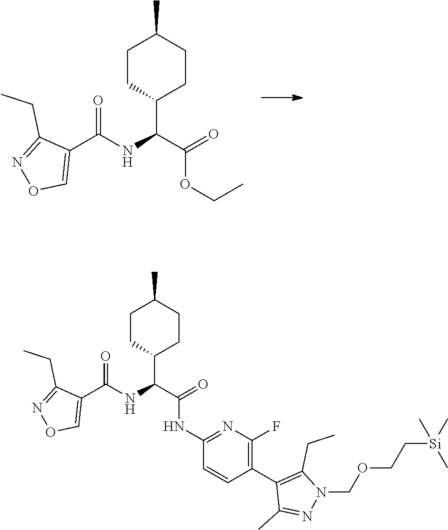 [SMILES: CCOC(=O)[C@@H](NC(=O)c1conc1CC)[C@H]1CC[C@H](C)CC1 ; CCc1nocc1C(=O)N[C@H](C(=O)Nc1ccc(-c2c(C)nn(COCC[Si](C)(C)C)c2CC)c(F)n1)[C@H]1CC[C@H](C)CC1]

According to the method of Preparation 27 the compound of Preparation 302 (200 mg, 0.63 mmol) was reacted with the compound of Preparation 39 (217 mg, 0.62 mmol) to afford the title compound as an off-white solid (300 mg, 76% yield). LCMS (METHOD 2) (ESI): m/z: 627 [M+H]⁺; 32%; RT=2.68 min (ACQUITY BEH C18 column, 0.1% TFA in water with MeCN).

### Preparation 305: ethyl (2S)-2-[(3-isopropylisoxazole-4-carbonyl)amino]-2-(4-methylcyclohexyl)acetate

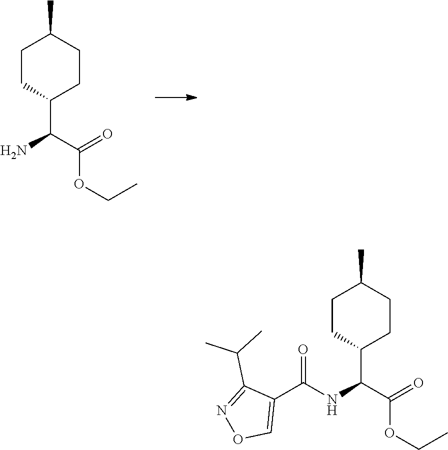 [SMILES: CCOC(=O)[C@@H](N)[C@H]1CC[C@H](C)CC1 ; CCOC(=O)[C@@H](NC(=O)c1conc1C(C)C)[C@H]1CC[C@H](C)CC1]

According to the method of Preparation 11 the compound of Preparation 298 (500 mg, 2.51 mmol) was reacted with 3-isopropylisoxazole-4-carboxylic acid (428 mg, 2.76 mmol) to afford the title compound as a colourless solid after flash chromatography (500 mg, 59% yield). 1H NMR (400 MHz, DMSO-d6) δ 8.7 (s, 1H), 6.7 (d, J=8.4 Hz, 1H), 4.69-4.66 (m, 1H), 4.26-4.20 (m, 2H), 3.45-3.41 (m, 1H), 1.80-1.60 (m, 4H), 1.41-1.32 (m, 1H), 1.45-1.33 (m, 6H), 1.30-1.22 (m, 4H), 1.21-1.10 (m, 2H), 0.867-0.962 (m, 5H); LCMS (METHOD 2) (ESI): m/z: 337 [M+H]⁺; 42%; RT=2.37 min (ACQUITY BEH C18 column, 0.05% FA in water with MeCN).

### Preparation 306: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-3-isopropyl-isoxazole-4-carboxamide

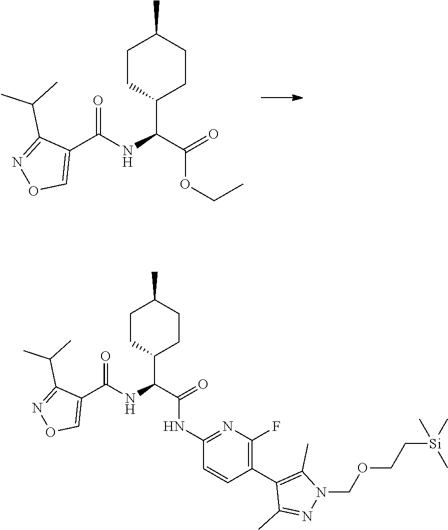 [SMILES: CCOC(=O)[C@@H](NC(=O)c1conc1C(C)C)[C@H]1CC[C@H](C)CC1 ; Cc1nn(COCC[Si](C)(C)C)c(C)c1-c1ccc(NC(=O)[C@@H](NC(=O)c2conc2C(C)C)[C@H]2CC[C@H](C)CC2)nc1F]

According to the method of Preparation 27 the compound of Preparation 305 (150 mg, 0.45 mmol) was reacted with the compound of Preparation 41 (150 mg, 0.45 mmol) to afford the title compound as a colourless solid (70 mg, 25% yield). LCMS (METHOD 2) (ESI): m/z: 627 [M+H]⁺; 92%; RT=2.69 min (ACQUITY BEH C18 column, 0.1% FA in water with MeCN).

### Preparation 307: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-3-isopropyl-isoxazole-4-carboxamide

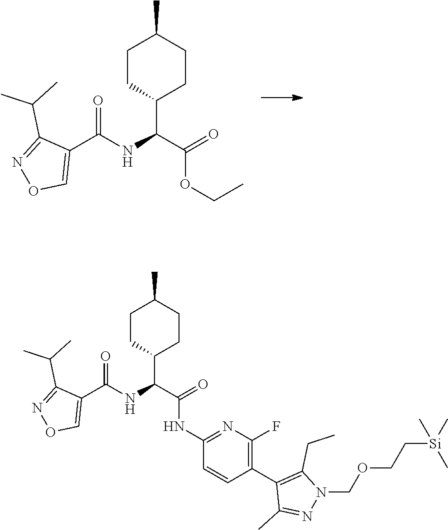 [SMILES: CCOC(=O)[C@@H](NC(=O)c1conc1C(C)C)[C@H]1CC[C@H](C)CC1 ; CCc1c(-c2ccc(NC(=O)[C@@H](NC(=O)c3conc3C(C)C)[C@H]3CC[C@H](C)CC3)nc2F)c(C)nn1COCC[Si](C)(C)C]

According to the method of Preparation 27 the compound of Preparation 302 (150 mg, 0.45 mmol) was reacted with the compound of Preparation 39 (156 mg, 0.45 mmol) to afford the title compound as an off-white solid (80 mg, 28% yield). LCMS (METHOD 2) (ESI): m/z: 641.4 [M+H]⁺; 91.7%; RT=3.2 min (ACQUITY BEH C18 column, 0.05% FA in water with MeCN).

### Preparation 308: ethyl (2S)-2-(4-methylcyclohexyl)-2-[(2-methylpyrazole-3-carbonyl)amino]acetate

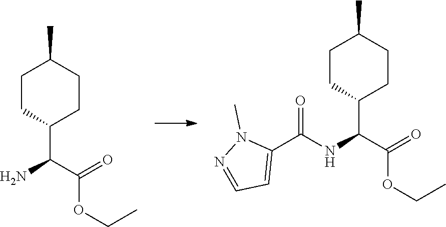 [SMILES: CCOC(=O)[C@@H](N)[C@H]1CC[C@H](C)CC1 ; CCOC(=O)[C@@H](NC(=O)c1ccnn1C)[C@H]1CC[C@H](C)CC1]

According to the method of Preparation 11 the compound of Preparation 298 (100 mg, 0.50 mmol) was reacted with 2-methylpyrazole-3-carboxylic acid (63 mg, 0.50 mmol) to afford the title compound after flash chromatography (90 mg, 58% yield). 1H NMR (300 MHz, DMSO-d6) δ 8.58 (br d, J=7.70 Hz, 1H) 7.49 (d, J=1.83 Hz, 1H) 6.89 (d, J=1.83 Hz, 1H) 5.17-5.52 (m, 1H) 3.99-4.19 (m, 2H) 2.69 (s, 3H) 1.49-1.83 (m, 5H) 1.35 (dd, J=6.60, 4.03 Hz, 3H) 1.02-1.31 (m, 3H) 0.85 (br d, J=6.24 Hz, 5H); LCMS (METHOD 2) (ESI): m/z: 308 [M+H]⁺; 89%; RT=2.43 min (ACQUITY BEH C18 column, 0.05% FA in water with MeCN).

### Preparation 309: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-methyl-pyrazole-3-carboxamide

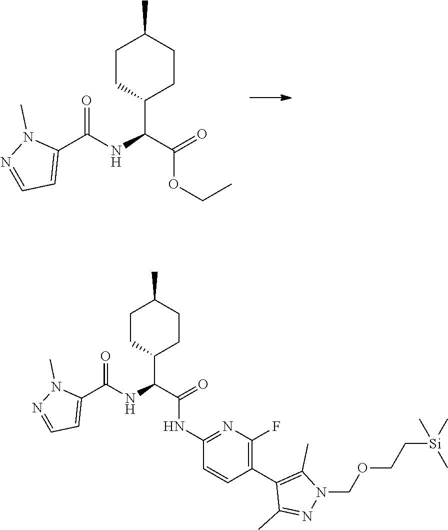 [SMILES: CCOC(=O)[C@@H](NC(=O)c1ccnn1C)[C@H]1CC[C@H](C)CC1 ; Cc1nn(COCC[Si](C)(C)C)c(C)c1-c1ccc(NC(=O)[C@@H](NC(=O)c2ccnn2C)[C@H]2CC[C@H](C)CC2)nc1F]

According to the method of Preparation 27 the compound of Preparation 308 (150 mg, 0.49 mmol) was reacted with the compound of Preparation 41 (180 mg, 0.51 mmol) to afford the title compound as a yellow oil (90 mg, 30% yield). 1H NMR (400 MHz, CDCl₃) δ 8.29 (s, 1H), 8.13 (dd, J=8.12, 1.14 Hz, 1H), 7.67 (dd, J=9.26, 8.28 Hz, 1H), 7.47 (d, J=2.07 Hz, 1H), 5.38 (s, 2H), 4.53-4.57 (m, 1H), 3.59-3.64 (m, 2H), 2.24 (s, 3H), 2.17 (s, 3H), 1.75-1.92 (m, 5H), 1.15-1.33 (m, 6H), 0.87-0.99 (m, 9H), 0.02 (s, 9H); LCMS (METHOD 2) (ESI): m/z: 598 [M+H]⁺; 95%; RT=2.8 min (ACQUITY BEH C18 column, 0.05% FA in water with MeCN).

### Preparation 310: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-methyl-pyrazole-3-carboxamide

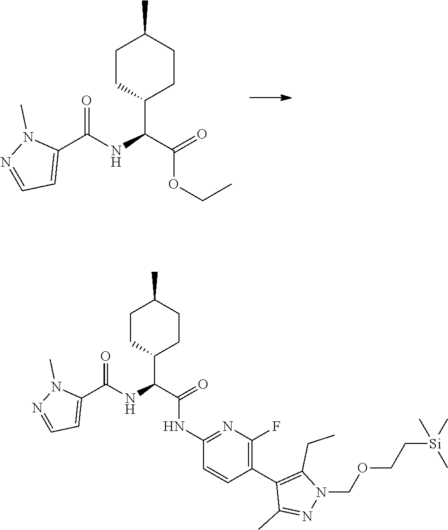 [SMILES: CCOC(=O)[C@@H](NC(=O)c1ccnn1C)[C@H]1CC[C@H](C)CC1 ; CCc1c(-c2ccc(NC(=O)[C@@H](NC(=O)c3ccnn3C)[C@H]3CC[C@H](C)CC3)nc2F)c(C)nn1COCC[Si](C)(C)C]

According to the method of Preparation 27 the compound of Preparation 308 (85 mg, 0.28 mmol) was reacted with the compound of Preparation 39 (93 mg, 0.28 mmol) to afford the title compound as a yellow oil (90 mg, 30% yield). LCMS (METHOD 2) (ESI): m/z: 612 [M+H]⁺; 69%; RT=2.93 min (ACQUITY BEH C18 column, 0.05% FA in water with MeCN).

### Preparation 311: ethyl (2S)-2-[(2-ethylpyrazole-3-carbonyl)amino]-2-(4-methylcyclohexyl)acetate

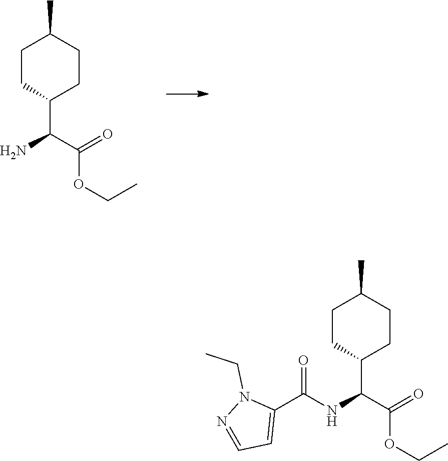 [SMILES: CCOC(=O)[C@@H](N)[C@H]1CC[C@H](C)CC1 ; CCOC(=O)[C@@H](NC(=O)c1ccnn1CC)[C@H]1CC[C@H](C)CC1]

According to the method of Preparation 11 the compound of Preparation 298 (80 mg, 0.40 mmol) was reacted with 2-ethylpyrazole-3-carboxylic acid (61 mg, 0.44 mmol) to afford the title compound after flash chromatography, as a yellow oil (101 mg, 78% yield). 1H NMR (300 MHz, CDCl₃) δ 8.57 (d, J=6.3 Hz, 1H), 7.47 (d, J=1.8 Hz, 1H), 6.96 (d, J=2.4 Hz, 1H), 4.43 (m, 2H), 4.21 (t, J=7.5 Hz, 1H), 4.16-4.10 (m, 2H), 1.57-1.50 (m, 5H), 1.30-1.12 (m, 9H), 0.95-80 (m, 5H). LCMS (METHOD 2) (ESI): m/z: 322 [M+H]⁺; 96%; RT=2.20 min (ACQUITY BEH C18 column, 0.05% FA in water with MeCN).

### Preparation 312: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-ethyl-pyrazole-3-carboxamide

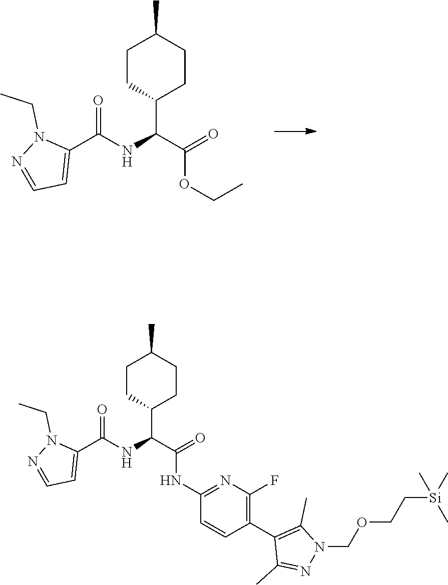 [SMILES: CCOC(=O)[C@@H](NC(=O)c1ccnn1CC)[C@H]1CC[C@H](C)CC1 ; CCn1nccc1C(=O)N[C@H](C(=O)Nc1ccc(-c2c(C)nn(COCC[Si](C)(C)C)c2C)c(F)n1)[C@H]1CC[C@H](C)CC1]

According to the method of Preparation 27 the compound of Preparation 311 (50 mg, 0.16 mmol) was reacted with the compound of Preparation 41 (52 mg, 0.16 mmol) to afford the title compound as a tacky gum (40 mg, 42% yield). 1H NMR (400 MHz, CDCl₃) δ 8.31 (s, 1H), 8.15 (dd, J=8.12, 1.14 Hz, 1H), 7.69 (dd, J=9.37, 8.17 Hz, 1H), 7.50 (d, J=2.07 Hz, 1H), 6.60-6.68 (m, 2H), 5.40 (s, 2H), 4.55-4.63 (m, 3H), 3.61-3.65 (m, 2H), 2.25 (s, 3H), 2.19 (s, 3H), 1.75-1.88 (m, 4H), 1.46 (t, J=7.14 Hz, 3H), 1.20-1.30 (m, 4H), 0.88-0.95 (m, 7H), 0.01 (s, 9H). LCMS (METHOD 2) (ESI): m/z: 612 [M+H]⁺; 95%; RT=2.96 min (ACQUITY BEH C18 column, 0.05% FA in water with MeCN).

### Preparation 313: 2-ethyl-N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]pyrazole-3-carboxamide

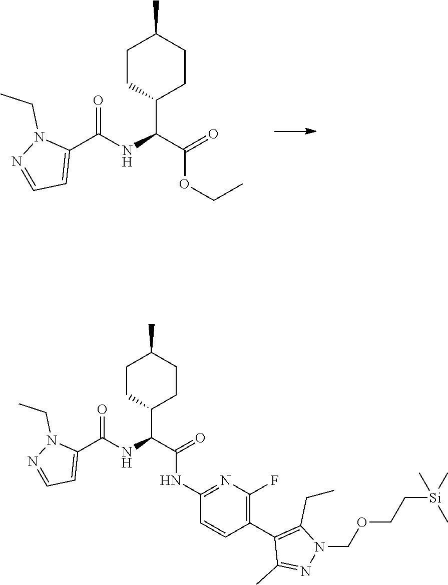 [SMILES: CCOC(=O)[C@@H](NC(=O)c1ccnn1CC)[C@H]1CC[C@H](C)CC1 ; CCc1c(-c2ccc(NC(=O)[C@@H](NC(=O)c3ccnn3CC)[C@H]3CC[C@H](C)CC3)nc2F)c(C)nn1COCC[Si](C)(C)C]

According to the method of Preparation 27 the compound of Preparation 311 (90 mg, 0.28 mmol) was reacted with the compound of Preparation 39 (98 mg, 0.28 mmol) to afford the title compound as a yellow oil (98 mg, 56% yield). 1H NMR (400 MHz, CDCl₃) δ 8.37-8.33 (m, 1H), 8.32 (d, J=8.0 Hz, 1H), 7.70-7.65 (m, 1H), 7.48 (d, J=2.0 Hz, 1H), 6.68-6.66 (m, 1H), 6.61 (d, J=2.4 Hz, 1H), 5.39 (s, 2H), 4.60-4.50 (m, 3H), 4.11-4.09 (m, 2H), 3.63-3.61 (m, 2H), 2.60-2.50 (m, 1H), 2.04 (s, 3H), 1.44-1.27 (m, 8H), 1.25-0.80 (m, 12H), -0.009 (s, 9H). LCMS (METHOD 2) (ESI): m/z: 626 [M+H]⁺; 82%; RT=2.95 min (ACQUITY BEH C18 column, 0.05% FA in water with MeCN).

### Preparation 314: ethyl (2S)-2-[(2-isopropylpyrazole-3-carbonyl)amino]-2-(4-methylcyclohexyl)acetate

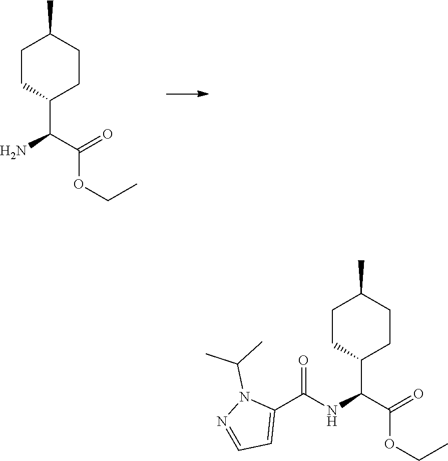 [SMILES: CCOC(=O)[C@@H](N)[C@H]1CC[C@H](C)CC1 ; CCOC(=O)[C@@H](NC(=O)c1ccnn1C(C)C)[C@H]1CC[C@H](C)CC1]

According to the method of Preparation 11 the compound of Preparation 298 (100 mg, 0.50 mmol) was reacted with 2-isopropylpyrazole-3-carboxylic acid (85 mg, 0.55 mmol) to afford the title compound after flash chromatography, as a brown oil (90 mg, 56% yield). LCMS (METHOD 2) (ESI): m/z: 336 [M+H]⁺; 86%; RT=2.63 min (ACQUITY BEH C18 column, 0.1% FA in water with MeCN).

### Preparation 315: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-isopropyl-pyrazole-3-carboxamide

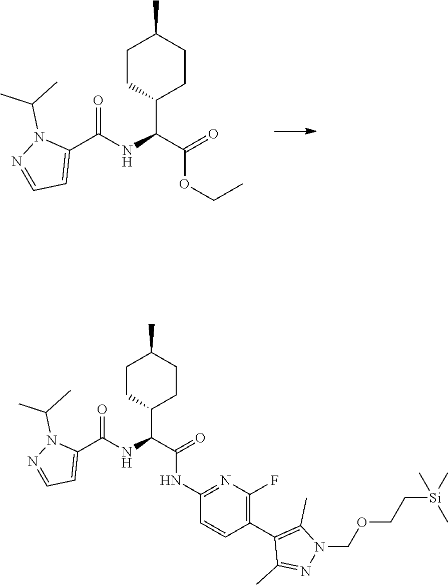 [SMILES: CCOC(=O)[C@@H](NC(=O)c1ccnn1C(C)C)[C@H]1CC[C@H](C)CC1 ; Cc1nn(COCC[Si](C)(C)C)c(C)c1-c1ccc(NC(=O)[C@@H](NC(=O)c2ccnn2C(C)C)[C@H]2CC[C@H](C)CC2)nc1F]

According to the method of Preparation 27 the compound of Preparation 314 (80 mg, 0.23 mmol) was reacted with the compound of Preparation 41 (80.2 mg, 0.23 mmol) to afford the title compound as a tacky gum (65 mg, 43% yield). LCMS (METHOD 2) (ESI): m/z: 626 [M+H]⁺; 81%; RT=2.99 min (ACQUITY BEH C18 column, 0.05% FA in water with MeCN).

### Preparation 316: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-isopropyl-pyrazole-3-carboxamide

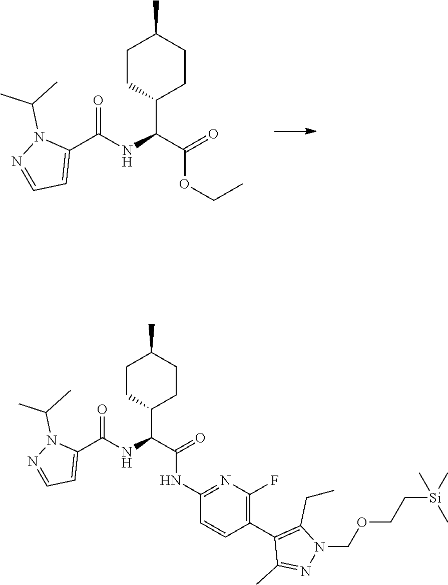 [SMILES: CCOC(=O)[C@@H](NC(=O)c1ccnn1C(C)C)[C@H]1CC[C@H](C)CC1 ; CCc1c(-c2ccc(NC(=O)[C@@H](NC(=O)c3ccnn3C(C)C)[C@H]3CC[C@H](C)CC3)nc2F)c(C)nn1COCC[Si](C)(C)C]

According to the method of Preparation 27 the compound of Preparation 314 (50 mg, 0.14 mmol) was reacted with the compound of Preparation 39 (52 mg, 0.14 mmol) to afford the title compound as a gum (100 mg, assume 100% yield). LCMS (METHOD 2) (ESI): m/z: 640 [M+H]⁺; 80%; RT=2.53 min (ACQUITY BEH C18 column, 0.1% FA in water with MeCN).

### Preparation 317: ethyl (2S)-2-(4-methylcyclohexyl)-2-[(2-propylpyrazole-3-carbonyl)amino]acetate

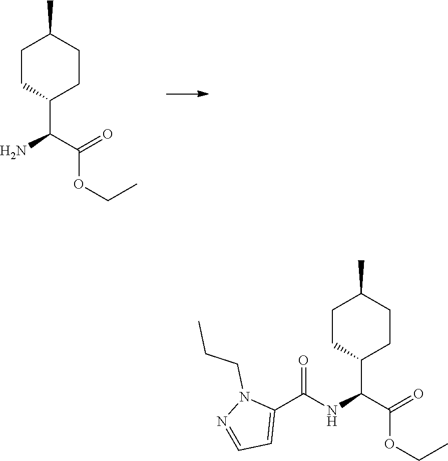 [SMILES: CCOC(=O)[C@@H](N)[C@H]1CC[C@H](C)CC1 ; CCCn1nccc1C(=O)N[C@H](C(=O)OCC)[C@H]1CC[C@H](C)CC1]

According to the method of Preparation 11 the compound of Preparation 298 (100 mg, 0.50 mmol) was reacted with 2-propylpyrazole-3-carboxylic acid (85 mg, 0.55 mmol) to afford the title compound, as a brown gum (90 mg, 53% yield). 1H NMR (300 MHz, DMSO-d6) δ 8.59 (br d, J=7.70 Hz, 1H) 7.48 (d, J=1.83 Hz, 1H) 6.95 (d, J=2.20 Hz, 1H) 4.39 (td, J=7.06, 3.85 Hz, 2H) 3.98-4.27 (m, 3H) 1.49-1.85 (m, 7H) 0.99-1.41 (m, 6H) 0.60-0.98 (m, 8H); LCMS (METHOD 2) (ESI): m/z: 336 [M+H]⁺; 98%; RT=2.61 min (ACQUITY BEH C18 column, 0.1% FA in water with MeCN).

### Preparation 318: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-propyl-pyrazole-3-carboxamide

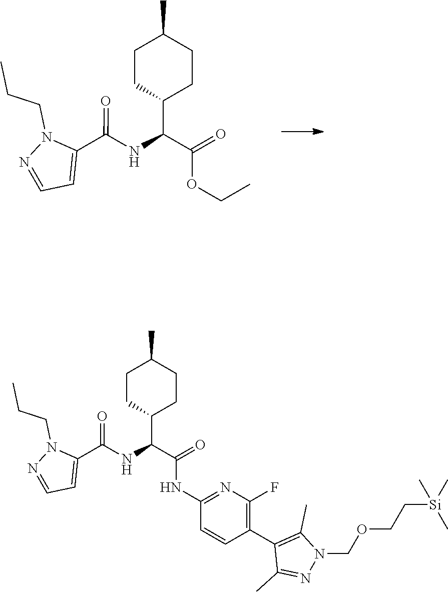 [SMILES: CCCn1nccc1C(=O)N[C@H](C(=O)OCC)[C@H]1CC[C@H](C)CC1 ; CCCn1nccc1C(=O)N[C@H](C(=O)Nc1ccc(-c2c(C)nn(COCC[Si](C)(C)C)c2C)c(F)n1)[C@H]1CC[C@H](C)CC1]

According to the method of Preparation 27 the compound of Preparation 317 (90 mg, 0.26 mmol) was reacted with the compound of Preparation 41 (90 mg, 0.26 mmol) to afford the title compound as a tacky gum (80 mg, 47% yield). 1H NMR (400 MHz, CDCl₃) δ 8.30 (s, 1H), 8.13 (d, J=8.07 Hz, 1H), 7.64-7.70 (m, 1H), 7.48 (d, J=2.07 Hz, 1H), 6.58-6.65 (m, 2H), 5.38 (s, 2H), 4.49-4.54 (m, 3H), 4.12 (q, J=7.08 Hz, 1H), 3.58-3.65 (m, 2H), 2.24 (s, 3H), 2.13 (s, 3H), 1.71-1.93 (m, 7H), 1.15-1.37 (m, 3H) 0.83-0.96 (m, 9H), -0.03 (s, 9H); LCMS (METHOD 2) (ESI): m/z:626 [M+H]⁺; 89%; RT=2.99 min (ACQUITY BEH C18 column, 0.05% FA in water with MeCN).

### Preparation 319: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-propyl-pyrazole-3-carboxamide

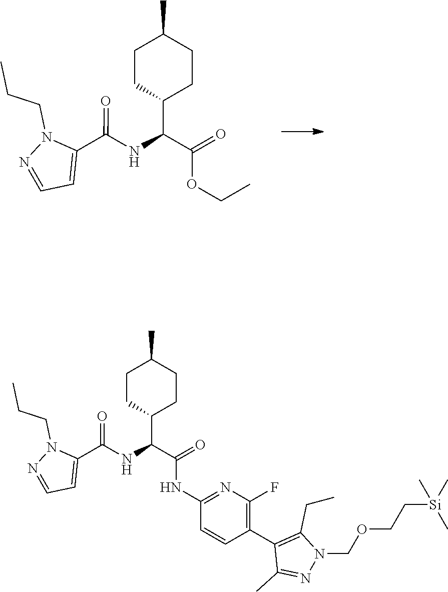 [SMILES: CCCn1nccc1C(=O)N[C@H](C(=O)OCC)[C@H]1CC[C@H](C)CC1 ; CCCn1nccc1C(=O)N[C@H](C(=O)Nc1ccc(-c2c(C)nn(COCC[Si](C)(C)C)c2CC)c(F)n1)[C@H]1CC[C@H](C)CC1]

According to the method of Preparation 27 the compound of Preparation 317 (150 mg, 0.45 mmol) was reacted with the compound of Preparation 39 (157 mg, 0.45 mmol) to afford the title compound as a gum (70 mg, 24% yield). LCMS (METHOD 2) (ESI): m/z: 640 [M+H]⁺; 70%; RT=2.73 min (ACQUITY BEH C18 column, 0.1% FA in water with MeCN).

### Preparation 320: ethyl (2S)-2-[[2-(2-methoxyethyl)pyrazole-3-carbonyl]amino]-2-(4-methylcyclohexyl)acetate

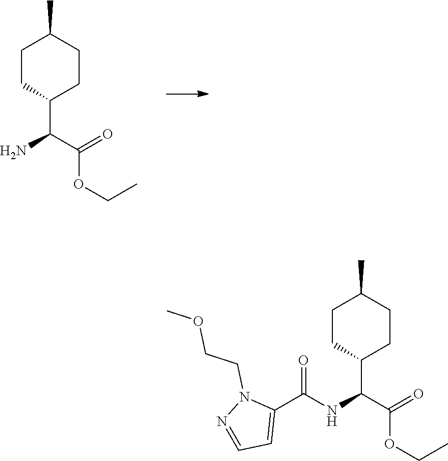 [SMILES: CCOC(=O)[C@@H](N)[C@H]1CC[C@H](C)CC1 ; CCOC(=O)[C@@H](NC(=O)c1ccnn1CCOC)[C@H]1CC[C@H](C)CC1]

According to the method of Preparation 11 the compound of Preparation 298 (100 mg, 0.50 mmol) was reacted with 2-(2-methoxyethyl)pyrazole-3-carboxylic acid (193 mg, 0.55 mmol) to afford the title compound, as a brown gum (90 mg, 50% yield). LCMS (METHOD 2) (ESI): m/z: 352.38 [M+H]⁺; 96%; RT=2.44 min (ACQUITY BEH C18 column, 0.1% FA in water with MeCN).

### Preparation 321: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-(2-methoxyethyl)pyrazole-3-carboxamide

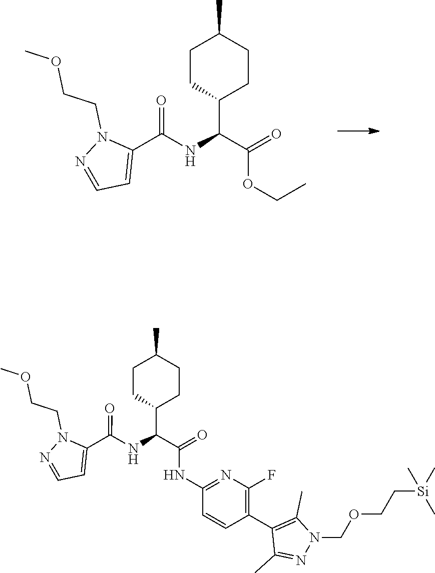

According to the method of Preparation 27 the compound of Preparation 320 (80 mg, 0.22 mmol) was reacted with the compound of Preparation 41 (77 mg, 0.22 mmol) to afford the title compound as a tacky gum (80 mg, 54% yield). 1H NMR (400 MHz, CDCl₃) δ 8.43 (s, 1H), 8.13 (dd, J=8.07, 1.31 Hz, 1H), 7.65 (dd, J=9.37, 8.17 Hz, 1H), 7.52 (d, J=1.96 Hz, 1H), 7.41 (d, J=8.17 Hz, 1H), 6.67 (d, J=1.96 Hz, 1H), 5.38 (s, 2H), 4.63-4.76 (m, 2H), 4.57 (t, J=7.47 Hz, 1H), 3.84 (t, J=5.18 Hz, 2H), 3.58-3.65 (m, 2H), 3.35 (s, 3H), 2.23 (s, 3H), 2.16 (s, 3H) 1.71-1.90 (m, 5H), 1.12-1.34 (m, 4H), 0.83-1.03 (m, 6H), 0.01 (s, 9H); LCMS (METHOD 2) (ESI): m/z: 642 [M+H]⁺; 95%; RT=2.88 min (ACQUITY BEH C18 column, 0.05% FA in water with MeCN).

### Preparation 322: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-(2-methoxyethyl)pyrazole-3-carboxamide

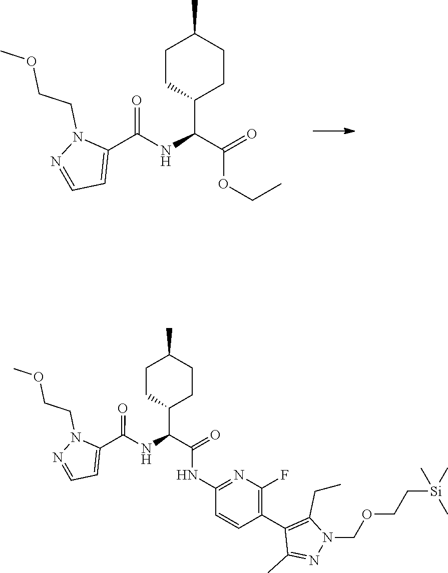

According to the method of Preparation 27 the compound of Preparation 320 (130 mg, 0.37 mmol) was reacted with the compound of Preparation 39 (130 mg, 0.37 mmol) to afford the title compound as a gum (90 mg, 37% yield). LCMS (METHOD 2) (ESI): m/z: 656 [M+H]⁺; 85%; RT=2.64 min (ACQUITY BEH C18 column, 0.1% FA in water with MeCN).

### Preparation 323: ethyl (2S)-2-[[2-(3-methoxypropyl)pyrazole-3-carbonyl]amino]-2-(4-methylcyclohexyl)acetate

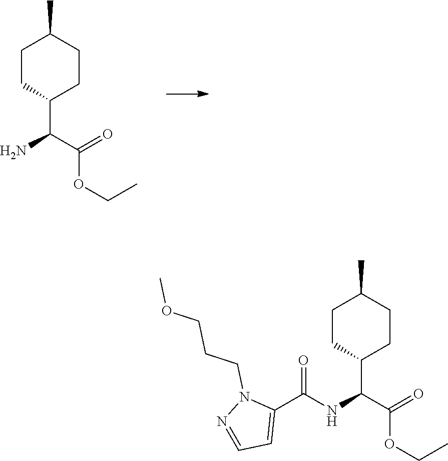 [SMILES: CCOC(=O)[C@@H](N)[C@H]1CC[C@H](C)CC1 ; CCOC(=O)[C@@H](NC(=O)c1ccnn1CCCOC)[C@H]1CC[C@H](C)CC1]

According to the method of Preparation 11 the compound of Preparation 298 (100 mg, 0.50 mmol) was reacted with the compound of Preparation 10 (101 mg, 0.55 mmol) to afford the title compound, as an off-white solid (90 mg, 46% yield). 1H NMR (300 MHz, DMSO-d6) δ 8.59 (br d, J=7.70 Hz, 1H) 7.49 (d, J=2.20 Hz, 1H) 6.96 (d, J=2.20 Hz, 1H) 4.46 (td, J=7.06, 1.28 Hz, 2H) 3.96-4.28 (m, 3H) 3.25 (t, J=6.42 Hz, 2H) 3.19 (s, 3H) 1.91 (br t, J=6.79 Hz, 2H) 1.53-1.80 (m, 5H) 1.02-1.36 (m, 6H) 0.72-0.96 (m, 5H); LCMS (METHOD 2) (ESI): m/z: 366.7 [M+H]⁺; 96%; RT=2.49 min (ACQUITY BEH C18 column, 0.1% FA in water with MeCN).

### Preparation 324: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-(3-methoxypropyl)pyrazole-3-carboxamide

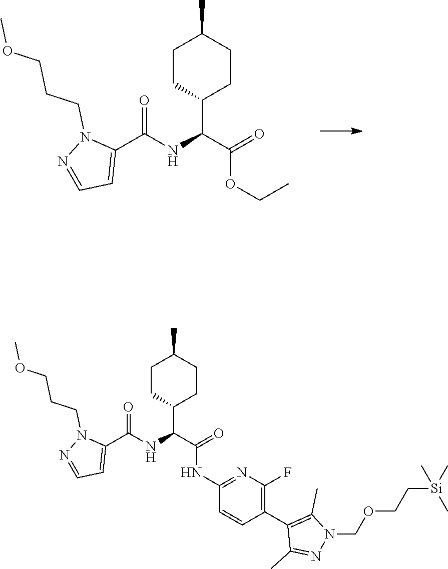

According to the method of Preparation 27 the compound of Preparation 323 (90 mg, 0.24 mmol) was reacted with the compound of Preparation 41 (82.8 mg, 0.24 mmol) to afford the title compound as a tacky gum (80 mg, 49% yield). 1H NMR (400 MHz, CDCl₃) δ 8.18 (s, 1H), 8.12 (d, J=7.96 Hz, 1H), 7.67 (dd, J=9.37, 8.17 Hz, 1H), 7.50 (d, J=2.07 Hz, 1H), 6.66 (d, J=8.39 Hz, 1H), 6.61 (d, J=2.07 Hz, 1H), 5.38 (s, 2H), 4.60-4.65 (m, J=7.03, 2H), 4.51-4.52 (m, 1H), 3.58-3.65 (m, 2H), 3.39 (t, J=6.27 Hz, 2H), 3.30 (s, 3H) 2.24 (s, 3H), 2.17 (s, 3H), 2.01-2.14 (m, 1H) 1.72-1.87 (m, 5H), 1.15-1.36 (m, 4H), 0.87-1.00 (m, 7H), 0.01 (s, 9H); LCMS (METHOD 2) (ESI): m/z: 656 [M+H]⁺; 91%; RT=2.92 min (ACQUITY BEH C18 column, 0.05% FA in water with MeCN).

### Preparation 325: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-(3-methoxypropyl)pyrazole-3-carboxamide

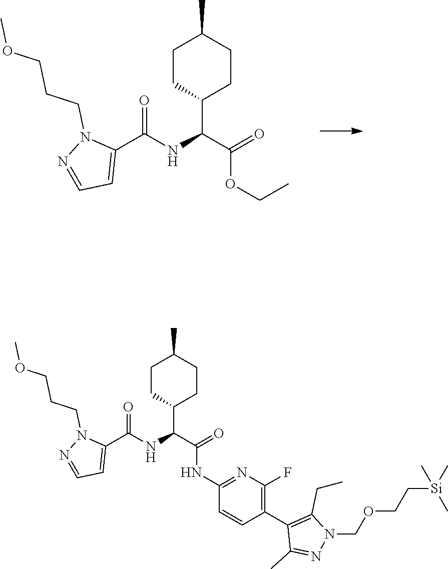

According to the method of Preparation 27 the compound of Preparation 320 (130 mg, 0.36 mmol) was reacted with the compound of Preparation 39 (125 mg, 0.36 mmol) to afford the title compound as a gum (90 mg, 37% yield). LCMS (METHOD 2) (ESI): m/z: 670.5 [M+H]⁺; 90.74%; RT=2.94 min (ACQUITY BEH C18 column, 0.1% FA in water with MeCN).

### Preparation 326: ethyl 2-(3-hydroxypropyl)pyrazole-3-carboxylate

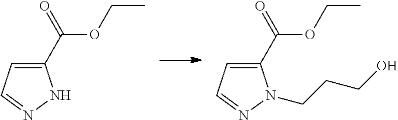

3-Bromopropan-1-ol (39.6 g, 267 mmol) was added to a mixture of ethyl 1H-pyrazole-5-carboxylate (25.0 g, 178 mmol) and K₂CO₃ (36.0 g, 267 mmol) in DMF (120 mL) at 0° C. On complete addition the reaction mixture was stirred at room temperature for 16 hours. The reaction mixture was diluted with H₂O (250 mL) and extracted with EtOAc (2×250 mL). The combined organic phase was dried over Na₂SO₄, filtered and concentrated in vacuo. The obtained crude compound was purified by silica column chromatography (230-400 mesh), eluting with EtOAc in hexane, to afford the title compound as a colourless gummy solid. (20.0 g, 64% yield). 1H NMR (400 MHz, DMSO-d6) δ 7.50 (d, J=2 Hz, 1H), 6.84 (d, J=2 Hz, 1H), 4.35 (q, J=8.4 Hz, 2H), 3.80-3.83 (m, 2H), 3.50-3.56 (m, 2H), 2.84 (t, J=6.4 Hz, 1H), 2.05-2.13 (m, 2H), 1.42 (t, J=6.4 Hz, 3H).

### Preparation 327: 2-(3-hydroxypropyl)pyrazole-3-carboxylic acid

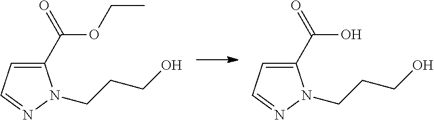

According to the method of Preparation 148 the compound of Preparation 326 (10.0 g, 50.5 mmol) was reacted to afford the crude title compound as an off-white solid (5.0 g, 58% yield). 1H NMR (400 MHz, DMSO-d6) δ 13.14 (br s, 1H), 7.51 (d, J=2 Hz, 1H), 6.71 (d, J 25=2.4 Hz, 1H), 4.56 (t, J=6.4 Hz, 2H), 3.45 (m, 2H), 1.91-1.85 (m, 2H); LCMS (METHOD 2) (ESI): m/z: 171 [M+H]⁺; 82% RT=2.13 min (ACQUITY BEH C18 column, 0.1% T FA in water with MeCN).

### Preparation 328: ethyl (2S)-2-[[2-(3-hydroxypropyl)pyrazole-3-carbonyl]amino]-2-(4-methylcyclohexyl)acetate

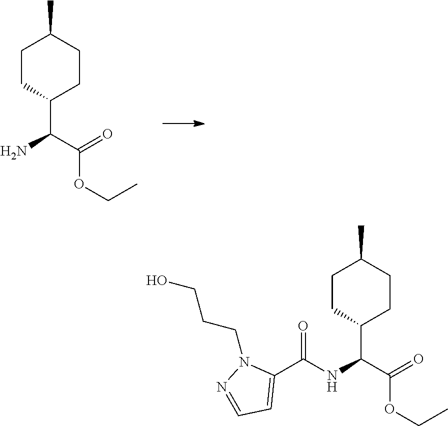 [SMILES: CCOC(=O)[C@@H](N)[C@H]1CC[C@H](C)CC1 ; CCOC(=O)[C@@H](NC(=O)c1ccnn1CCCO)[C@H]1CC[C@H](C)CC1]

According to the method of Preparation 11 the compound of Preparation 298 (200 mg, 1.0 mmol) was reacted with the compound of Preparation 327 (187 mg, 1.10 mmol) to afford the title compound as a colourless oil (260 mg, 76% yield). LCMS (METHOD 2) (ESI): m/z:352 [M+H]⁺; 85%; RT=2.41 min (ACQUITY BEH C18 column, 0.05% FA in water with MeCN).

### Preparation 329: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-(3-hydroxypropyl)pyrazole-3-carboxamide

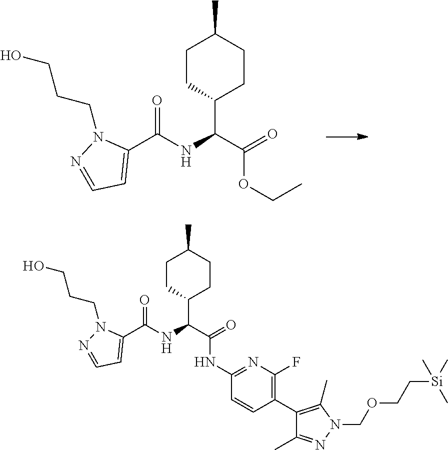 [SMILES: CCOC(=O)[C@@H](NC(=O)c1ccnn1CCCO)[C@H]1CC[C@H](C)CC1 ; Cc1nn(COCC[Si](C)(C)C)c(C)c1-c1ccc(NC(=O)[C@@H](NC(=O)c2ccnn2CCCO)[C@H]2CC[C@H](C)CC2)nc1F]

According to the method of Preparation 27 the compound of Preparation 328 (140 mg, 0.42 mmol) was reacted with the compound of Preparation 41 (143 mg, 0.42 mmol) to afford the title compound as a gummy oil (38 mg, 15% yield). LCMS (METHOD 2) (ESI): m/z: 642 [M+H]⁺; 89%; RT=2.96 min (ACQUITY BEH C18 column, 0.05% FA in water with MeCN).

### Preparation 331: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-(3-hydroxypropyl)pyrazole-3-carboxamide

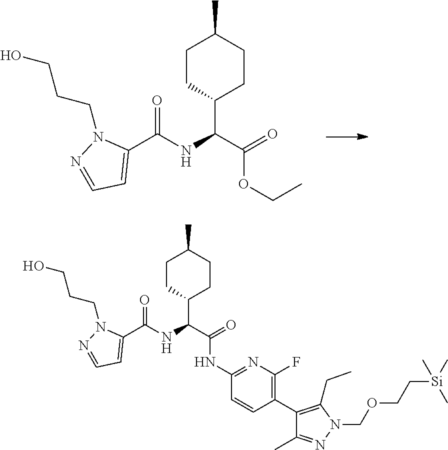 [SMILES: CCOC(=O)[C@@H](NC(=O)c1ccnn1CCCO)[C@H]1CC[C@H](C)CC1 ; CCc1c(-c2ccc(NC(=O)[C@@H](NC(=O)c3ccnn3CCCO)[C@H]3CC[C@H](C)CC3)nc2F)c(C)nn1COCC[Si](C)(C)C]

According to the method of Preparation 27 the compound of Preparation 328 (120 mg, 0.34 mmol) was reacted with the compound of Preparation 39 (120 mg, 0.34 mmol) to afford the title compound as a viscous oil (90 mg, 40% yield). 1H NMR (400 MHz, CDCl₃) 8.69 (m, 1H), 8.12 (d, J=8.0 Hz, 1H), 7.68 (t, J=9.2 Hz, 1H), 7.50 (d, J=2.0 Hz, 1H), 8.22 (d, J=8.8 Hz, 1H), 6.63 (d, J=2.0 Hz, 1H), 5.40 (s, 2H), 4.12-4.10 (m, 1H), 4.70-4.52 (m, 4H), 3.52-3.49 (m, 2H), 2.59-2.52 (m, 2H), 2.07 (br s, 3H), 1.87-1.12 (m, 10H), 1.01-0.07 (m, 11H), -0.01 (s, 9H). LCMS (METHOD 2) (ESI): m/z: 656 [M+H]⁺; 87%; RT=2.98 min (ACQUITY BEH C18 column, 0.05% FA in water with MeCN).

### Preparation 332: ethyl (2S)-2-(4-methylcyclohexyl)-2-[[2-(2-methylsulfanylethyl)pyrazole-3-carbonyl]amino]acetate

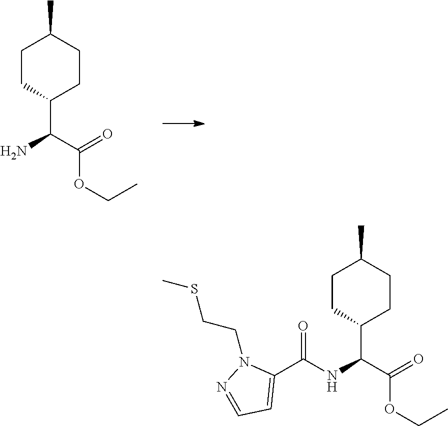 [SMILES: CCOC(=O)[C@@H](N)[C@H]1CC[C@H](C)CC1 ; CCOC(=O)[C@@H](NC(=O)c1ccnn1CCSC)[C@H]1CC[C@H](C)CC1]

According to the method of Preparation 11 the compound of Preparation 298 (100 mg, 0.50 mmol) was reacted with the compound of Preparation 148 (193 mg, 0.55 mmol) to afford the title compound, as a brown gum (90 mg, 50% yield). 1H NMR (400 MHz, DMSO-d6) δ 8.64 (br d, J=7.70 Hz, 1H) 7.51 (d, J=1.83 Hz, 1H) 6.98 (d, J=2.20 Hz, 1H) 4.63 (td, J=6.97, 2.57 Hz, 2H) 4.22 (t, J=7.70 Hz, 1H) 4.11 (qd, J=7.03, 2.75 Hz, 2H) 2.79 (t, J=6.97 Hz, 2H) 2.00 (s, 3H) 1.47-1.82 (m, 5H) 0.97-1.39 (m, 6H) 0.67-0.95 (m, 5H); LCMS (METHOD 2) (ESI): m/z: 368.3 [M+H]⁺; 99.48%; RT=3.54 min (ACQUITY BEH C18 column, 0.1% FA in water with MeCN).

### Preparation 333: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-(2-methylsulfanylethyl)pyrazole-3-carboxamide

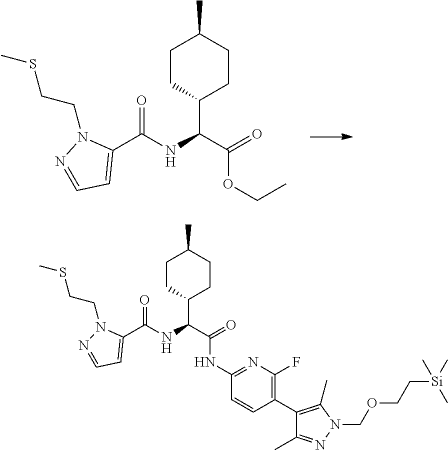

According to the method of Preparation 27 the compound of Preparation 332 (100 mg, 0.27 mmol) was reacted with the compound of Preparation 41 (96 mg, 0.28 mmol) to afford the title compound as a gummy oil (70 mg, 39% yield). 1H NMR (400 MHz, CDCl₃) δ 8.24 (s, 1H), 8.07-8.16 (m, 1H), 7.64-7.70 (m, 1H), 7.52 (d, J=1.96 Hz, 1H), 6.59-6.70 (m, 2H), 5.38 (s, 2H), 4.77 (t, J=6.98 Hz, 2H), 4.50-4.57 (m, 1H), 3.58-3.64 (m, 2H), 2.93 (t, J=6.98 Hz, 2H), 2.24 (s, 3H), 2.17 (s, 3H), 2.05-2.10 (m, 3H), 1.74-1.96 (m, 5H), 1.15-1.38 (m, 4H), 0.87-1.01 (m, 6H), 0.01-0.03 (m, 9H); LCMS (METHOD 2) (ESI): m/z: 658 [M+H]⁺; 96%; RT=2.96 min (ACQUITY BEH C18 column, 0.05% FA in water with MeCN); Chiral HPLC: 91.61% (RT: 3.72 min),Column: CHIRALPAK IG (4.6*150 mm) 5 μm, Co-Solvent: 0.5% DEA in Methanol, Column Temperature: 30° C., Flow: 3 mL/min, Outlet Pressure: 100 bar. PDA 220.0 nm.

### Preparation 334: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-(2-methylsulfanylethyl)pyrazole-3-carboxamide

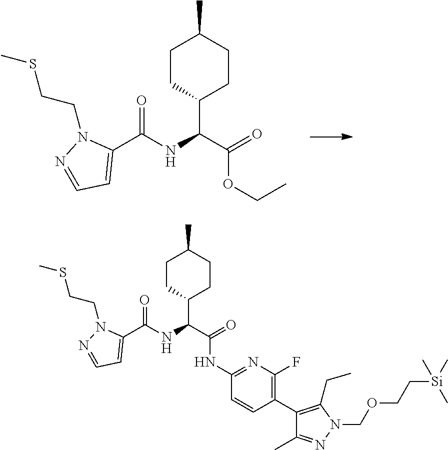

According to the method of Preparation 27 the compound of Preparation 320 (140 mg, 0.38 mmol) was reacted with the compound of Preparation 39 (134 mg, 0.38 mmol) to afford the title compound as a gum (90 mg, 35% yield). LCMS (METHOD 2) (ESI): m/z: 672.43 [M+H]⁺; 51%; RT=2.98 min (ACQUITY BEH C18 column, 0.1% FA in water with MeCN).

### Preparation 335: ethyl (2S)-2-(4-methylcyclohexyl)-2-[[2-(2-methylsulfinylethyl)pyrazole-3-carbonyl]amino]acetate

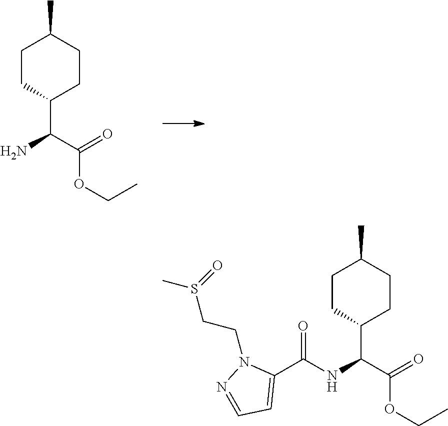 [SMILES: CCOC(=O)[C@@H](N)[C@H]1CC[C@H](C)CC1 ; CCOC(=O)[C@@H](NC(=O)c1ccnn1CCS(C)=O)[C@H]1CC[C@H](C)CC1]

According to the method of Preparation 11 the compound of Preparation 298 (100 mg, 0.50 mmol) was reacted with the compound of Preparation 150 (112 mg, 0.55 mmol) to afford the title compound, as a colourless solid (125 mg, 65% yield). 1H NMR (300 MHz, DMSO-d6) δ 8.75 (d, J=7.8 Hz, 1H), 7.56 (d, J=1.8 Hz, 1H), 7.09 (d, J=2.1 Hz, 1H), 4.86 (t, J=7.6 Hz, 1H), 4.25 (t, J=7.5 Hz, 1H), 4.18-4.07 (m, 2H), 3.60 (t, J=6.9 Hz, 2H), 2.95 (s, 3H), 1.78-1.60 (m, 5H), 1.35-1.02 (m, 7H), 0.95-0.80 (m, 5H). LCMS (METHOD 2) (ESI): m/z: 384 [M+H]⁺; 93%; RT=1.87 min (ACQUITY BEH C18 column, 0.05% FA in water with MeCN).

### Preparation 336: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-(2-methylsulfinylethyl)pyrazole-3-carboxamide

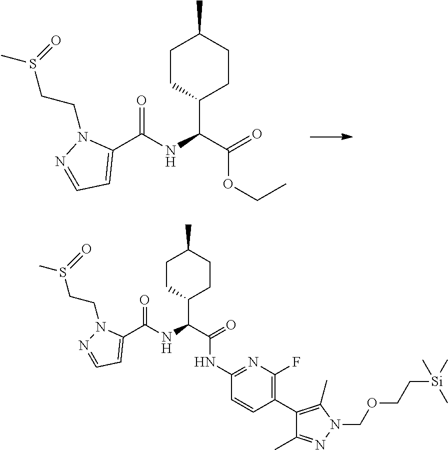

According to the method of Preparation 27 the compound of Preparation 335 (110 mg, 0.28 mmol) was reacted with the compound of Preparation 41 (96 mg, 0.28 mmol) to afford the title compound as a gummy oil (98 mg, 50% yield). LCMS (METHOD 2) (ESI): m/z: 674 [M+H]⁺; 68%; RT=2.71 min (ACQUITY BEH C18 column, 0.05% FA in water with MeCN).

### Preparation 337: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-(2-methylsulfinylethyl)pyrazole-3-carboxamide

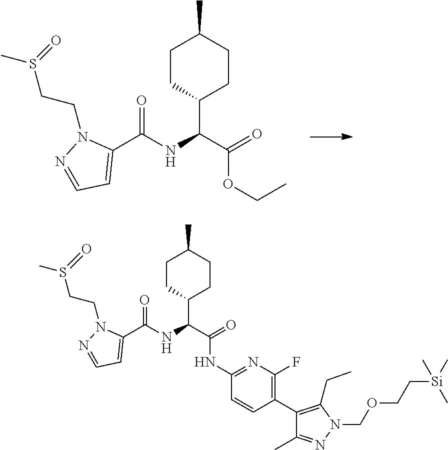

According to the method of Preparation 27 the compound of Preparation 335 (130 mg, 0.33 mmol) was reacted with the compound of Preparation 39 (119 mg, 0.33 mmol) to afford the title compound as a gum (105 mg, 45% yield). LCMS (METHOD 2) (ESI): m/z: 688 [M+H]⁺; 89%; RT=2.78 min (ACQUITY BEH C18 column, 0.05% FA in water with MeCN).

### Preparation 338: ethyl (2S)-2-(4-methylcyclohexyl)-2-[[2-(2-methylsulfonylethyl)pyrazole-3-carbonyl]amino]acetate

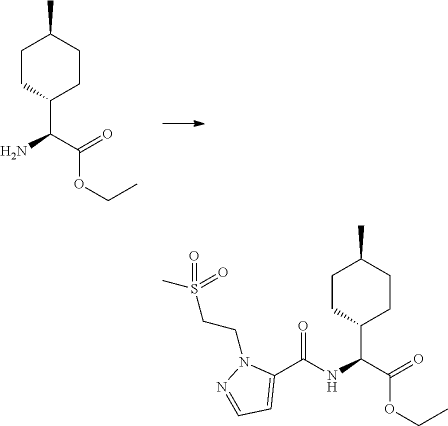 [SMILES: CCOC(=O)[C@@H](N)[C@H]1CC[C@H](C)CC1 ; CCOC(=O)[C@@H](NC(=O)c1ccnn1CCS(C)(=O)=O)[C@H]1CC[C@H](C)CC1]

According to the method of Preparation 11 the compound of Preparation 298 (60 mg, 0.30 mmol) was reacted with the compound of Preparation 152 (72 mg, 0.33 mmol) to afford the title compound, as a colourless solid (90 mg, 75% yield). 1H NMR (300 MHz, DMSO-d6) δ 8.68 (d, J=8.07 Hz, 1H) 7.57 (d, J=1.83 Hz, 1H) 7.09 (d, J=1.83 Hz, 1H) 4.71-4.95 (m, 2H) 4.25 (t, J=7.70 Hz, 1H) 4.02-4.19 (m, 2H) 3.60 (t, J=7.15 Hz, 2H) 2.95 (s, 3H) 1.69 (d, J=16.14 Hz, 5H) 0.98-1.36 (m, 6H) 0.55-0.97 (m, 5H); LCMS (METHOD 2) (ESI): m/z: 400.32 [M+H]⁺; 99%; RT=2.38 min (ACQUITY BEH C18 column, 0.1% FA in water with MeCN).

### Preparation 339: N-[(1S)-2-[[5-[3,5-dimethyl-1-(2-trimethylsilylethoxymethyl)pyrazol-4-yl]-6-fluoro-2-pyridyl]amino]-1-(4-methylcyclohexyl)-2-oxo-ethyl]-2-(2-methylsulfonylethyl)pyrazole-3-carboxamide

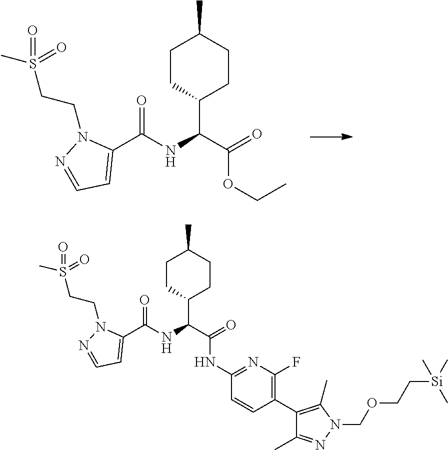

According to the method of Preparation 27 the compound of Preparation 338 (120 mg, 0.30 mmol) was reacted with the compound of Preparation 41 (101 mg, 0.30 mmol) to afford the title compound as a gummy oil (54 mg, 26% yield). LCMS (METHOD 2) (ESI): m/z: 690 [M+H]⁺; 94%; RT=2.78 min (ACQUITY BEH C18 column, 0.05% FA in water with MeCN).

### Preparation 340: N-[(1S)-2-[[5-[5-ethyl-3-methyl-1-(2-trimethylsilylethoxymethyl)pyrazol-4-yl]-6-fluoro-2-pyridyl]amino]-1-(4-methylcyclohexyl)-2-oxo-ethyl]-2-(2-methylsulfonylethyl)pyrazole-3-carboxamide

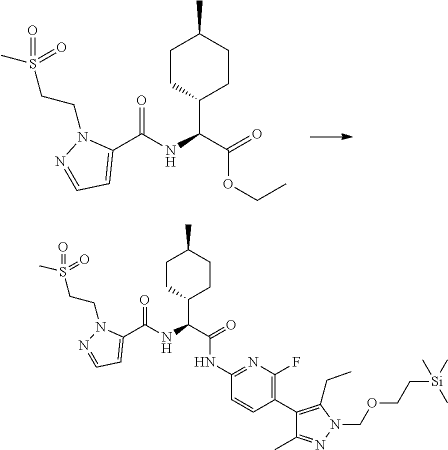

According to the method of Preparation 27 the compound of Preparation 338 (90 mg, 0.23 mmol) was reacted with the compound of Preparation 39 (79 mg, 0.23 mmol) to afford the title compound as a gum (60 mg, 38% yield). LCMS (METHOD 2) (ESI): m/z: 704.41 [M+H]⁺; 73%; RT=2.9 min (ACQUITY BEH C18 column, 0.1% FA in water with MeCN).

### Preparation 341: 2-[[3,5-dimethyl-4-(4-nitrophenyl)pyrazol-1-yl]methoxy]ethyl-trimethyl-silane

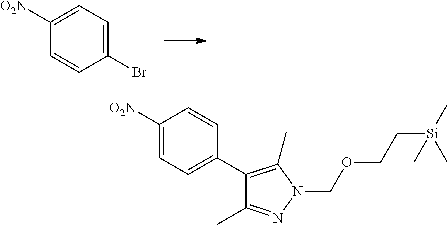

According to the method of Preparation 22, 4-bromonitrobenzene (1.55 g, 7.66 mmol) was reacted with the compound of Preparation 21 (2.70 g, 7.66 mmol) to afford the title compound (1.89 g, 71%). 1H NMR (300 MHz, DMSO-d6) δ 8.39-8.19 (m, 2H), 7.69-7.40 (m, 2H), 5.39 (s, 2H), 3.64-3.53 (m, 2H), 2.33 (s, 3H), 2.21 (s, 3H), 0.92-0.76 (m, 2H), −0.03 (s, 9H); LCMS (ES): m/z 384.3 [M+H]⁺, RT=0.95 min.

### Preparation 342: 4-[3,5-dimethyl-1-(2-trimethylsilylethoxymethyl)pyrazol-4-yl]aniline

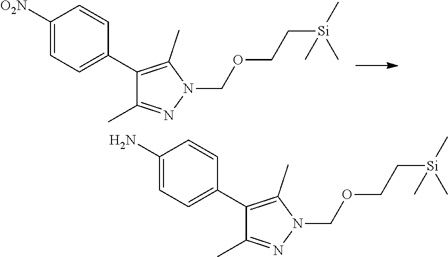

10% Pd/C (188 mg) was added to a solution of the compound of Preparation 341 (1.88 g, 5.41 mmol) in MeOH (30 mL) and placed under hydrogen at atmospheric pressure. After 1 hour the catalyst was filtered off, washing with MeOH, and the filtrate was concentrated in vacuo to give the title compound as a colourless solid (1.67 g, 97%). 1H NMR (300 MHz, DMSO-d6) δ 6.96-6.85 (m, 2H), 6.65-6.57 (m, 2H), 5.30 (s, 2H), 5.03 (s, 2H), 3.59-3.48 (m, 2H), 2.20 (s, 3H), 2.08 (s, 3H), 0.83 (dd, J=8.4, 7.4 Hz, 2H), −0.04 (s, 9H); LCMS (ES): m/z 318.4 [M+H]⁺, RT=0.80 min.

### Preparation 343: tert-butyl N-[(1S)-1-(dicyclopropylmethyl)-2-[4-[3,5-dimethyl-1-(2-trimethylsilylethoxymethyl)pyrazol-4-yl]anilino]-2-oxo-ethyl]carbamate

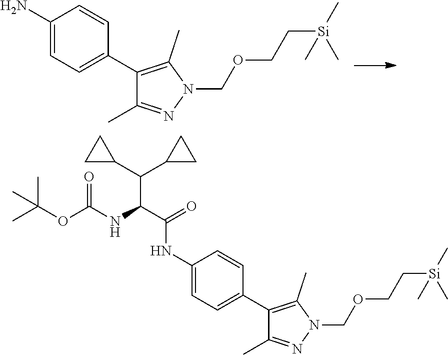

HATU (5.70 g, 15.0 mmol) was added to a solution of the compound of Preparation 36 (2.95 g, 11.0 mmol), the compound of Preparation 342 (3.83 g, 12.0 mmol) and DIPEA (3.82 mL, 2.83 g, 21.9 mmol) in dry DMF (15 mL) and the mixture was stirred at room temperature for 18 hours. The reaction was poured into water (250 mL) and extracted with Et₂O (3×80 mL). The combined organic extracts were dried (Na₂SO₄), and concentrated in vacuo. The residue was purified by column chromatography (silica, eluting with 0-100% EtOAc in heptane) to give the title compound as a pale yellow solid (5.97 g, 96%). LCMS (METHOD 3) (ES): m/z 567.5 [M−H]⁻, RT=1.01 min.

### Preparation 344: (2S)-2-amino-3,3-dicyclopropyl-N-[4-[3,5-dimethyl-1-(2-trimethylsilylethoxymethyl)pyrazol-4-yl]phenyl]propenamide hydrochloride

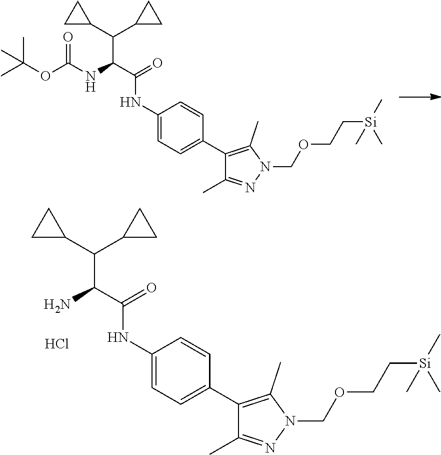 [SMILES: Cc1nn(COCC[Si](C)(C)C)c(C)c1-c1ccc(NC(=O)[C@@H](NC(=O)OC(C)(C)C)C(C2CC2)C2CC2)cc1 ; Cc1nn(COCC[Si](C)(C)C)c(C)c1-c1ccc(NC(=O)[C@@H](N)C(C2CC2)C2CC2)cc1 ; Cl]

The compound of Preparation 344 (5.97 g, 10.5 mmol) was dissolved in 1M hydrogen chloride in MeOH (50 mL) and the reaction was stirred at room temperature for 3 hours. The mixture was concentrated in vacuo to give the title compound as an off-white solid (5.30 g, 100%). LCMS (METHOD 3) (ES): m/z 469.3 [M+H]⁺, RT=0.71 min.

### Preparation 345: 3-(4-nitrophenyl)pentane-2,4-dione

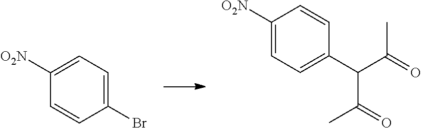 [SMILES: O=[N+]([O-])c1ccc(Br)cc1 ; CC(=O)C(C(C)=O)c1ccc([N+](=O)[O-])cc1]

1-Bromo-4-nitro-benzene (5.0 g, 20.1 mmol), pentane-2,4-dione (4.01 g, 40.2 mmol) and K₂CO₃ (6.92 g, 50.2 mmol) were taken in dry DMSO (100 mL) and purged with Argon gas for 15 min. CuI (0.381 g, 2.00 mmol) was added, followed by (S)-Proline (0.461 g, 4.01 mmol). The resulting reaction mixture was stirred at 70° C. for 16 hours. The reaction mixture was diluted with water (100 mL) and extracted with EtOAc (2×100 mL). The combined organic layers were washed with water (100 mL) and brine (100 mL), dried (Na₂SO₄) and concentrated under reduced pressure. The residue was purified by column chromatography (silica, eluting with 5% EtOAc in petroleum ether) to give the title compound as a yellow solid (1.8 g, 40%). 1H NMR (CDCl₃, 400 MHz) δ 16.76 (s, 1H), 8.27 (d, J=8.8 Hz, 2H), 7.39 (d, J=8.8 Hz, 2H), 1.90 (s, 6H); LCMS (METHOD 3) (ES): m/z=220 [M−H]⁻, RT=1.98 min.

### Preparation 346: 3,5-dimethyl-4-(4-nitrophenyl)-1H-pyrazole

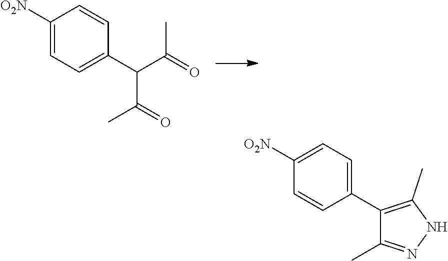 [SMILES: CC(=O)C(C(C)=O)c1ccc([N+](=O)[O-])cc1 ; Cc1n[nH]c(C)c1-c1ccc([N+](=O)[O-])cc1]

Hydrazine hydrate (56.5 mL, 1130 mmol) was added to a stirred solution of the compound of Preparation 345 (50 g, 226 mmol) in EtOH (1 L) at room temperature. The reaction mixture was then heated at 70° C. for 6 hours. The reaction mixture was concentrated under reduced pressure and the residue was diluted with water (1 L) and stirred at room temperature for 20 minutes. The precipitate was filtered, washed with cold water (300 mL) and hexane (300 mL). The solid was dried to give the title compound as a yellow solid (35 g, 71%). 1H NMR (400 MHz, DMSO-d6) δ 12.55 (br s, 1H), 8.26-8.23 (d, J=8.8 Hz, 2H), 7.59-7.57 (d, J=9.2 Hz, 2H), 2.29 (s, 3H), 2.23 (s, 3H); LCMS (ES): m/z=218 [M+H]⁺, RT=5.62 min.

### Preparation 347: tert-butyl 3,5-dimethyl-4-(4-nitrophenyl)pyrazole-1-carboxylate

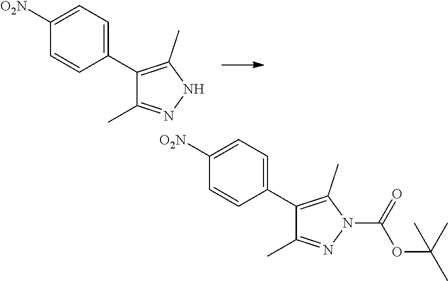

DMAP (112.5 mg, 0.92 mmol) was added to a suspension of the compound of Preparation 346 (2.0 g, 9.21 mmol) and tert-butoxycarbonyl tert-butyl carbonate (2.11 g, 9.67 mmol) in MeCN (20 mL) and stirred at room temperature for 2 hours. H₂O (10 mL) was added and the reaction mixture was cooled to 0° C. The precipitate was collected, washed and dried in vacuo to afford the title compound as a pale yellow solid. (2.33 g, 80% yield). 1H NMR (400 MHz, DMSO-d6) δ 8.36-8.23 (m, 2H), 7.67-7.53 (m, 2H), 2.44 (s, 3H), 2.19 (s, 3H), 1.59 (s, 9H).

### Preparation 348: tert-butyl 4-(4-aminophenyl)-3,5-dimethyl-pyrazole-1-carboxylate

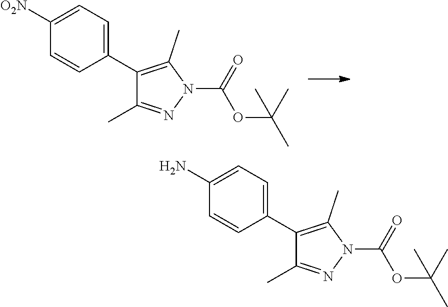

5% Pd/C (220 mg) was added to a solution of the compound of Preparation 347 (2.2 g, 6.90 mmol) in MeOH (22 mL) and the reaction mixture was stirred under 4 bars pressure of hydrogen at room temperature for 1 hour. The reaction mixture was filtered through Celite and the catalyst was washed with MeOH. The filtrate was concentrated in vacuo to give the title compound as an off-white solid. (1.92 g, 96%). 1H NMR (400 MHz, DMSO-d6) δ 6.99-6.86 (m, 2H), 6.69-6.56 (m, 2H), 5.17 (s, 2H), 2.35 (s, 3H), 2.10 (s, 3H), 1.57 (s, 9H).

### Preparation 349: (2S)-2-(benzyloxycarbonylamino)-3,3-dicyclopropyl-propanoic acid

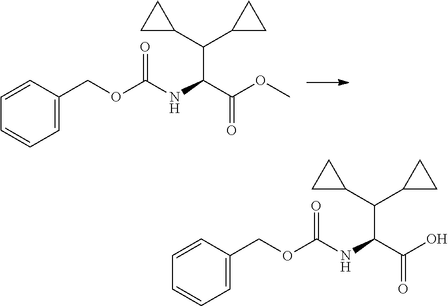 [SMILES: COC(=O)[C@@H](NC(=O)OCc1ccccc1)C(C1CC1)C1CC1 ; O=C(N[C@H](C(=O)O)C(C1CC1)C1CC1)OCc1ccccc1]

NaOH (4M aq. solution, 31.3 mmol) was added to a solution of the compound of Preparation 160a (4.96 g, 15.6 mmol) in MeOH (20 mL) and DCM (20 mL) and the reaction mixture was stirred at room temperature for 16 hours. H₂O (50 mL) was added and the mixture was extracted with TBME (2×100 mL). The aqueous phase was acidified to pH 2 with 4M hydrogen chloride (aq. solution), then extracted with EtOAc (3×100 mL). The combined EtOAc layers were dried over MgSO₄, filtered and concentrated in vacuo to leave the title compound as a colourless solid (4.56 g, 96% yield). 1H NMR (600 MHz, CDCl₃) δ 7.41-7.28 (m, 5H), 5.53 (d, J=9.2 Hz, 1H), 5.13 (s, 2H), 4.64 (dd, J=9.2, 2.6 Hz, 1H), 0.84-0.67 (m, 3H), 0.62-0.33 (m, 4H), 0.33-0.05 (m, 4H).

### Preparation 350: tert-butyl 4-[4-[[(2S)-2-(benzyloxycarbonylamino)-3,3-dicyclopropyl-propanoyl]amino]phenyl]-3,5-dimethyl-pyrazole-1-carboxylate

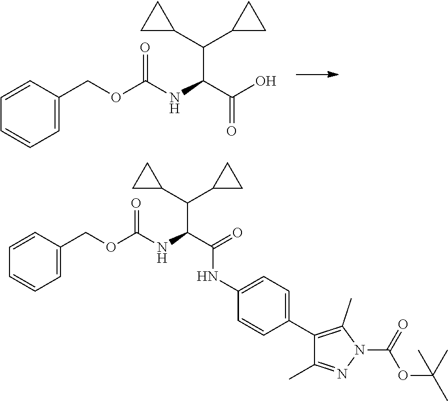

T3P (1.38 g, 2.17 mmol) was added to a mixture of the compound of Preparation 349 (264 mg, 0.87 mmol), the compound of Preparation 348 (250 mg, 0.87 mmol) and N-methylimidazole (0.173 mL, 2.17 mmol) in EtOAc (10 mL) at 3° C. and stirred for 3 hours. The reaction mixture was diluted with EtOAc (10 mL), washed successively with H₂O (2×5 mL), saturated aq. NaHCO₃ solution (5 mL), saturated brine solution, then concentrated in vacuo to leave the title compound as a colourless solid. (423 mg, 85% yield). 1H NMR (600 MHz, DMSO-d6) δ 10.07 (s, 1H), 7.68 (d, J=8.3 Hz, 2H), 7.48 (d, J=9.1 Hz, 1H), 7.41-7.34 (m, 4H), 7.34-7.28 (m, 1H), 7.27-7.18 (m, 3H), 5.07 (s, 2H), 4.40 (dd, J=9.1, 6.9 Hz, 1H), 2.39 (s, 3H), 2.14 (s, 3H), 1.58 (s, 10H), 0.90 (tt, J=8.7, 3.5 Hz, 1H), 0.78 (q, J=6.8, 5.1 Hz, 1H), 0.60 (td, J=9.4, 6.8 Hz, 1H), 0.46 (dtd, J=12.4, 8.5, 6.9, 3.8 Hz, 1H), 0.36 (tt, J=8.8, 3.8 Hz, 1H), 0.31-0.12 (m, 6H).

### Preparation 351: tert-butyl 4-[4-[[(2S)-2-amino-3,3-dicyclopropyl-propanoyl]amino]phenyl]-3,5-dimethyl-pyrazole-1-carboxylate

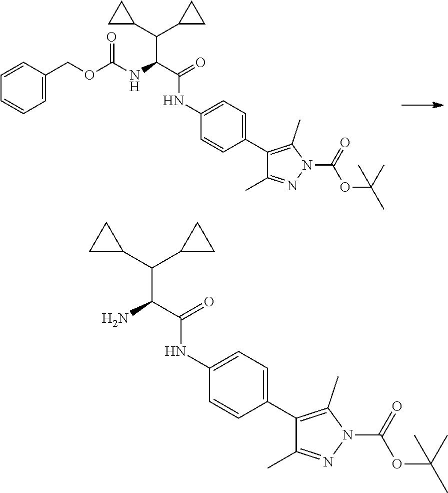 [SMILES: Cc1nn(C(=O)OC(C)(C)C)c(C)c1-c1ccc(NC(=O)[C@@H](NC(=O)OCc2ccccc2)C(C2CC2)C2CC2)cc1 ; Cc1nn(C(=O)OC(C)(C)C)c(C)c1-c1ccc(NC(=O)[C@@H](N)C(C2CC2)C2CC2)cc1]

According to the method of Preparation 161 the compound of Preparation 350 (25.0 g, 39.0 mmol) was reacted to afford the title compound as a gum (15.19 g, 89% yield). 1H NMR (400 MHz, CDCl₃) δ 9.72 (s, 1H), 7.72-7.58 (m, 2H), 7.22-7.10 (m, 2H), 3.64 (d, J=2.9 Hz, 1H), 2.45 (s, 3H), 2.24 (s, 3H), 1.66 (s, 9H), 1.05 (td, J=9.1, 2.9 Hz, 1H), 0.88-0.57 (m, 3H), 0.57-0.19 (m, 7H).

### Preparation 352: 2-(2-hydroxy-1-methyl-ethyl)pyrazole-3-carboxylic acid

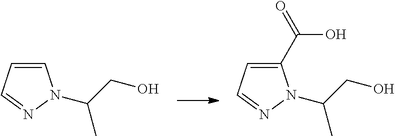

n-Butyllithium (2.5 M in hexanes, 15 mL 37.7 mmol) was added dropwise to a solution of 2-pyrazol-1-ylpropan-1-ol (1.90 g, 15.1 mmol) and TMEDA (4.52 mL, 3.50 g, 30.1 mmol) in dry THF (50 mL) at 0° C. under argon. The resulting suspension was stirred for 30 minutes at 0° C. and CO₂ gas was then passed through the solution for 10 minutes. The reaction mixture was concentrated in vacuo. Hydrogen chloride (4M aq. soln) was slowly added until the pH was between 3 and 4 and the mixture was extracted with EtOAc (3×40 mL). The combined organic extracts were dried over Na₂SO₄, filtered and concentrated in vacuo. The resulting pale solid was triturated with ether:hexane (1:1, 20 mL), filtered and dried in vacuo to give the title compound (1.60 g, 62%) as an off-white solid. 1H NMR (300 MHz, DMSO-d6) δ 13.16 (s, 1H), 7.54 (dd, J=1.9, 0.5 Hz, 1H), 6.78 (d, J=1.9 Hz, 1H), 5.71-5.18 (m, 1H), 3.69 (dd, J=10.6, 7.5 Hz, 1H), 3.59 (dd, J=10.6, 5.9 Hz, 1H), 1.34 (d, J=6.7 Hz, 3H).

### Preparation 353: ethyl 2-(2-hydroxy-1-methyl-ethyl)pyrazole-3-carboxylate

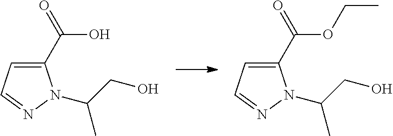

K₂CO₃ (406 mg, 2.94 mmol) was added to a solution of the compound of Preparation 352 (500 mg, 2.94 mmol) in DMF (10 mL) and vigorously stirred at room temperature for 30 minutes. To this was added ethyl iodide (0.24 mL, 2.94 mmol) and the reaction mixture was stirred for 16 hours at room temperature. The reaction mixture was neutralised with 5M hydrogen chloride solution, diluted with H₂O and extracted with DCM (3×50 mL). The combined organic layers were dried over Na₂SO₄ and concentrated in vacuo, to afford the crude title compound as a brown oil. (583 mg, assume 100% yield). Material used without further purification. 1H NMR (400 MHz, CDCl₃) δ 7.52 (d, J=2.0 Hz, 1H), 6.85 (d, J=2.0 Hz, 1H), 5.45 (td, J=6.5, 3.3 Hz, 1H), 4.35 (q, J=7.1 Hz, 2H), 4.04-3.88 (m, 2H), 1.49 (d, J=6.7 Hz, 3H), 1.38 (t, J=7.1 Hz, 3H).

### Preparation 354: ethyl 2-[1-methyl-2-(p-tolylsulfonyloxy)ethyl]pyrazole-3-carboxylate

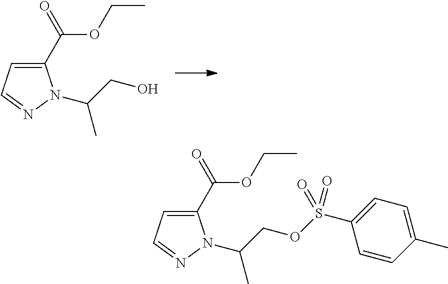 [SMILES: CCOC(=O)c1ccnn1C(C)CO ; CCOC(=O)c1ccnn1C(C)COS(=O)(=O)c1ccc(C)cc1]

Tosyl chloride (841 mg, 4.41 mmol) was added to a solution of the compound of Preparation 353 (583 mg, 2.94 mmol) and DABCO (660 mg, 5.88 mmol) in DCM (10 mL) and stirred at room temperature for 16 hours. The reaction mixture was quenched with 5M hydrogen chloride solution and extracted with TBME (2×50 mL). The combined organic layer was washed with H₂O, saturated brine solution, then dried over Na₂SO₄, filtered and concentrated in vacuo. The obtained crude compound was purified by silica column chromatography (230-400 mesh), eluting with EtOAc in heptane, to afford the title compound as a colourless oil (626 mg, 60% yield). 1H NMR (400 MHz, CDCl₃) δ 7.70-7.56 (m, 2H), 7.40 (d, J=2.0 Hz, 1H), 7.29 (d, J=8.0 Hz, 2H), 6.77 (d, J=1.9 Hz, 1H), 5.79-5.62 (m, 1H), 4.46-4.15 (m, 4H), 2.44 (s, 3H), 1.46 (d, J=6.8 Hz, 3H), 1.38 (t, J=7.1 Hz, 3H).

### Preparation 355: ethyl 2-(1-methyl-2-methylsulfanyl-ethyl)pyrazole-3-carboxylate

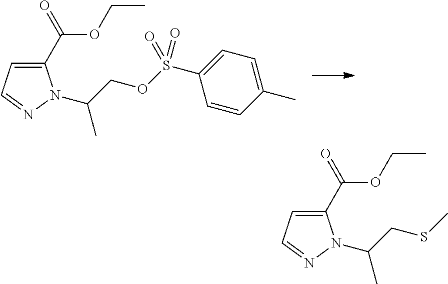 [SMILES: CCOC(=O)c1ccnn1C(C)COS(=O)(=O)c1ccc(C)cc1 ; CCOC(=O)c1ccnn1C(C)CSC]

Sodium methanethiolate (249 mg, 3.56 mmol) was added to a solution of the compound of Preparation 354 (626 mg, 1.78 mmol) in DMF (10 mL) at room temperature and stirred for 16 hours. The reaction mixture was diluted with TBME (40 mL) and washed with H₂O (2×10 mL). The organic layer was washed with saturated brine solution, dried over Na₂SO₄, filtered and concentrated in vacuo to afford the title compound (338 mg, 83% yield). 1H NMR (600 MHz, CDCl₃) δ 7.53 (d, J=1.9 Hz, 1H), 6.83 (d, J=1.9 Hz, 1H), 5.68-5.54 (m, 1H), 4.35 (q, J=7.2 Hz, 2H), 3.08-2.80 (m, 2H), 2.00 (s, 3H), 1.60 (d, J=6.7 Hz, 3H), 1.38 (t, J=7.1 Hz, 3H).

### Preparation 356: 2-(1-methyl-2-methylsulfanyl-ethyl)pyrazole-3-carboxylic acid

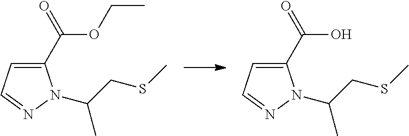

According to the method of Preparation 148 the compound of Preparation 355 (153 mg, 0.67 mmol) was reacted to afford the crude title compound (100 mg, 74% yield). 1H NMR (400 MHz, CDCl₃) δ 7.61 (t, J=1.9 Hz, 1H), 6.99 (d, J=1.9 Hz, 1H), 5.69-5.50 (m, 1H), 3.13-2.80 (m, 2H), 2.00 (s, 3H), 1.63 (d, J=6.7 Hz, 3H). LCMS (METHOD 3) (ES): m/z 199.1 [M−H]⁻, RT=0.47 min.

### Preparation 357: tert-butyl 4-[4-[[(2S)-3,3-dicyclopropyl-2-[[2-(1-methyl-2-methylsulfanyl-ethyl)pyrazole-3-carbonyl]amino]propanoyl]amino]phenyl]-3,5-dimethyl-pyrazole-1-carboxylate

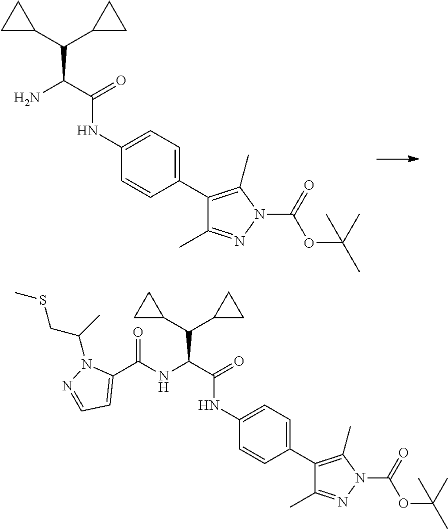

According to the method of Preparation 11 the compound of Preparation 351 (20.0 mg, 0.045 mmol) was reacted with the compound of Preparation 356 (9.1 mg, 0.045 mmol) to afford the title compound after prep. acidic HPLC (28.0 mg, assume 100% yield). LCMS (METHOD 3) (ES): m/z 619.3 [M−H]⁻, RT=0.94 min.

### Preparation 358: tert-butyl 4-[4-[[(2S)-3,3-dicyclopropyl-2-[[2-[(1-methylazetidin-3-yl)methyl]pyrazole-3-carbonyl]amino]propanoyl]amino]phenyl]-3,5-dimethyl-pyrazole-1-carboxylate

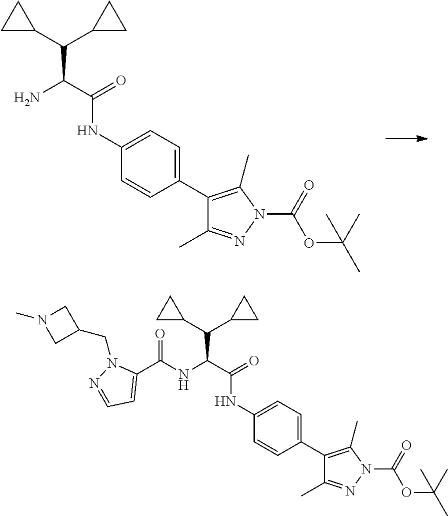

According to the method of Preparation 11 the compound of Preparation 351 (20.0 mg, 0.045 mmol) was reacted with the compound of Preparation 216 (12.8 mg, 0.045 mmol) to afford the title compound after prep. acidic HPLC (23.3 mg, 83% yield). LCMS (METHOD 3) (ES): m/z 616.5 [M+H]⁺, RT=0.94 min.

### Preparation 359: tert-butyl 4-[4-[[(2S)-2-[[2-[(1-tert-butoxycarbonylazetidin-3-yl)methyl]pyrazole-3-carbonyl]amino]-3,3-dicyclopropyl-propanoyl]amino]phenyl]-3,5-dimethyl-pyrazole-1-carboxylate

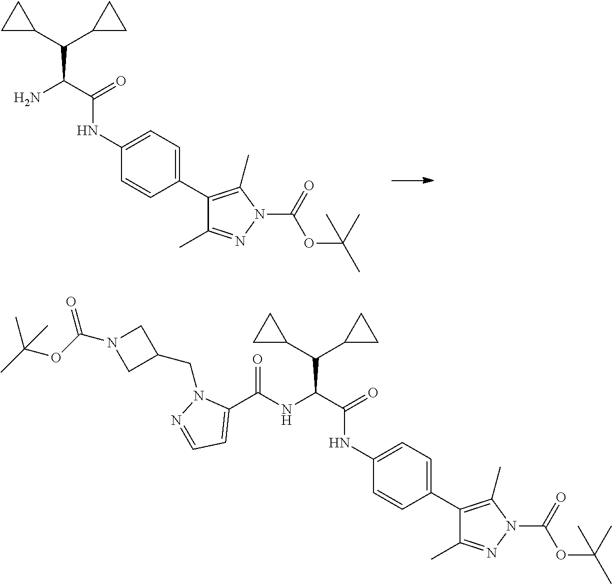

According to the method of Preparation 11 the compound of Preparation 351 (20.0 mg, 0.045 mmol) was reacted with the compound of Preparation 218 (12.8 mg, 0.045 mmol) to afford the title compound after prep. acidic HPLC (27.9 mg, 87% yield). LCMS (METHOD 3) (ES): m/z 702.7 [M+H]⁺, RT=0.94 min.

### Preparation 360: (2S)-2-amino-3,3-dicyclopropyl-N-[4-(3,5-dimethyl-1H-pyrazol-4-yl)phenyl]propenamide

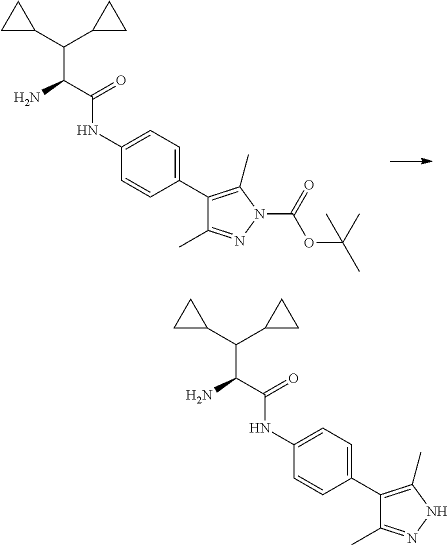 [SMILES: Cc1nn(C(=O)OC(C)(C)C)c(C)c1-c1ccc(NC(=O)[C@@H](N)C(C2CC2)C2CC2)cc1 ; Cc1n[nH]c(C)c1-c1ccc(NC(=O)[C@@H](N)C(C2CC2)C2CC2)cc1]

Hydrogen chloride (4.0 mL, 4M solution in 1,4-dioxane) was added to a solution of the compound of Preparation 351 (250 mg, 0.57 mmol) in MeOH (5 mL) and stirred at room temperature for 18 hours. The reaction mixture was concentrated in vacuo and the residue was dissolved in H₂O (10 mL). The resultant solution was basified to pH>10 with 4M aq. NaOH. The precipitate was filtered, washed with H₂O and dried in vacuo to afford the title compound as a colourless solid (171 mg, 88% yield). LCMS (METHOD 3) (ES): m/z 339.3 [M+H]⁺, RT=0.49 min.

### Preparation 361: ethyl 2-(oxetan-3-ylmethyl)pyrazole-3-carboxylate

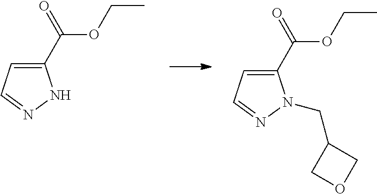

According to the method of Preparation 147, ethyl 1H-pyrazole-5-carboxylate (253 mg, 1.81 mmol) was reacted with oxetan-3-ylmethanol (223 mg, 2.53 mmol) to afford the title compound as an off-white solid (220 mg, 60% yield). 1H NMR (400 MHz, CDCl₃) δ 7.47 (d, J=2.0 Hz, 1H), 6.84 (d, J=2.0 Hz, 1H), 4.88 (d, J=7.3 Hz, 2H), 4.78 (dd, J=7.9, 6.4 Hz, 2H), 4.58 (t, J=6.3 Hz, 2H), 4.34 (q, J=7.1 Hz, 2H), 3.65-3.43 (m, 1H), 1.38 (t, J=7.1 Hz, 3H); LCMS (METHOD 3) (ES): m/z 211.1 [M+H]⁺, RT=0.55 min.

### Preparation 362: Cesium 2-(oxetan-3-ylmethyl)pyrazole-3-carboxylate

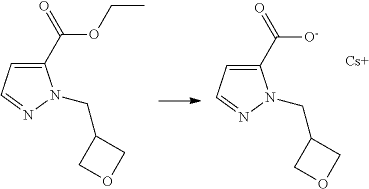

Cesium hydroxide (60 mg, 0.36 mmol) was added to a solution of the compound of Preparation 361 (54.0 mg, 0.26 mmol) in MeOH (2 mL) and the mixture was stirred at room temperature for 1 hour, then concentrated in vacuo and used directly in the next step without purification (80 mg, assume 100% yield); LCMS (METHOD 3) (ES): m/z 183.1 [M+H]⁺, RT=0.29 min.

### Preparation 363: N-[(1S)-1-(dicyclopropylmethyl)-2-[4-[3,5-dimethyl-1-(2-trimethylsilylethoxymethyl)pyrazol-4-yl]anilino]-2-oxo-ethyl]-2-(2-methylsulfanylethyl)pyrazole-3-carboxamide

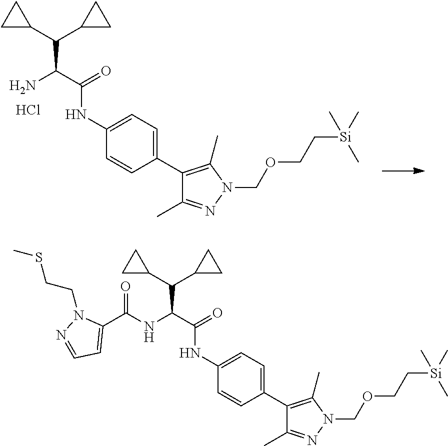

According to the method of Preparation 11 the compound of Preparation 344 (80.0 mg, 0.15 mmol) was reacted with the compound of Preparation 148 (29.0 mg, 0.15 mmol) to afford the title compound as an off-white solid (70 mg, 50% yield). 1H NMR (300 MHz, DMSO-d6) δ 10.27 (s, 1H), 8.56 (br d, J=8.80 Hz, 1H), 7.70 (d, J=8.80 Hz, 2H), 7.56 (d, J=2.20 Hz, 1H), 7.25 (d, J=8.80 Hz, 2H), 7.09 (d, J=1.83 Hz, 1H), 5.38 (s, 2H), 4.98-4.77 (m, 1H), 4.76-4.60 (m, 2H), 3.59 (t, J=7.89 Hz, 2H), 2.85 (t, J=7.15 Hz, 2H), 2.52 (s, 3H), 2.29 (s, 3H), 2.03 (m, 3H), 1.08-0.62 (m, 5H), 0.57-0.10 (m, 8H), 0.09 (s, 9H); LCMS (METHOD 2) (ESI): m/z: 637 [M+H]⁺; 93%; RT=2.50 min (ACQUITY BEH C18 column, 0.05% FA in water with MeCN).

### Preparation 364: N-[(1S)-1-(dicyclopropylmethyl)-2-[4-[3,5-dimethyl-1-(2-trimethylsilylethoxymethyl)pyrazol-4-yl]anilino]-2-oxo-ethyl]-2-(2-methylsulfinylethyl)pyrazole-3-carboxamide

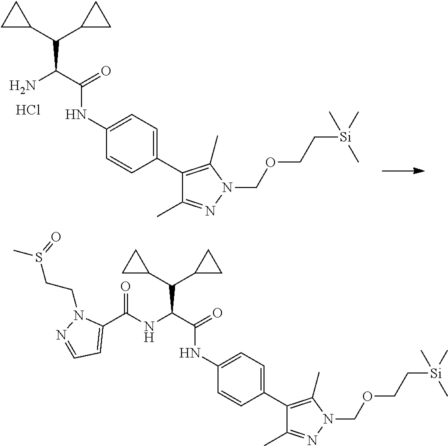

According to the method of Preparation 11 the compound of Preparation 344 (100 mg, 0.19 mmol) was reacted with the compound of Preparation 150 (40 mg, 0.19 mmol) to afford the title compound as an off-white solid (60 mg, 46% yield). 1H NMR (400 MHz, DMSO-d6) δ 10.24 (s, 1H), 8.59 (m, 1H), 7.71 (d, J=8.40 Hz, 2H), 7.59 (m, 1H), 7.24 (d, J=8.40 Hz, 2H), 7.15 (d, J=1.2 Hz, 1H), 5.37 (s, 2H), 4.84-4.95 (m, 4H), 3.69-3.78 (s, 3H), 3.51-3.61 (m, 2H), 2.28 (s, 3H), 2.12 (s, 3H), 0.50-1.50 (m, 7H), 0.16-0.55 (m, 8H), 0.02 (m, 9H); LCMS (METHOD 2) (ESI): m/z: 653 [M+H]⁺; 91%; RT=2.23 min, (ACQUITY BEH C18 column, 0.05% FA in water with MeCN). Chiral HPLC: 41% (RT: 3.18 min) & 41% (RT: 5.35 min), Column: Chiralcel OX-H (4.6*250)mm, 5 mic, Co-Solvent: 0.5% DEA in Methanol (50%), Column Temperature: 30° C., Flow: 4 mL/min, Outlet Pressure: 100 bar.

### Preparation 365: N-[(1S)-1-(dicyclopropylmethyl)-2-[4-[3,5-dimethyl-1-(2-trimethylsilylethoxymethyl)pyrazol-4-yl]anilino]-2-oxo-ethyl]-2-(3-methylsulfanylpropyl)pyrazole-3-carboxamide

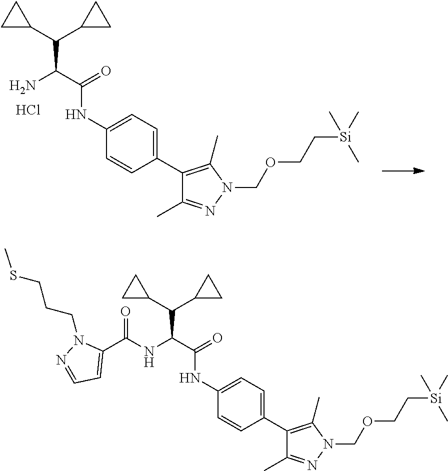

According to the method of Preparation 11 the compound of Preparation 344 (130 mg, 0.25 mmol) was reacted with the compound of Preparation 154 (51.5 mg, 0.15 mmol) to afford the title compound as an off-white solid (150 mg, 89% yield). LCMS (METHOD 2) (ESI): m/z: 651.6 [M+H]⁺; 76%; RT=2.53 min (ACQUITY BEH C18 column, 0.1% FA in water with MeCN).

### Preparation 366: N-[(1S)-1-(dicyclopropylmethyl)-2-[4-[3,5-dimethyl-1-(2-trimethylsilylethoxymethyl)pyrazol-4-yl]anilino]-2-oxo-ethyl]-2-(3-methylsulfinylpropyl)pyrazole-3-carboxamide

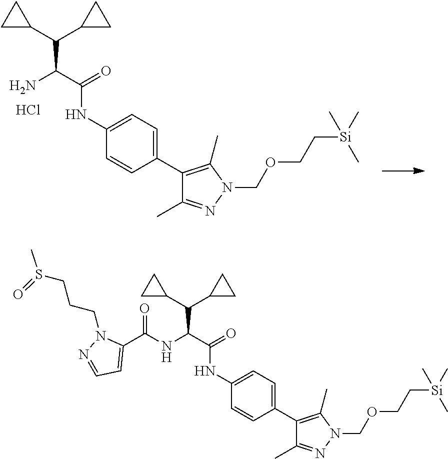 [SMILES: Cc1nn(COCC[Si](C)(C)C)c(C)c1-c1ccc(NC(=O)[C@@H](N)C(C2CC2)C2CC2)cc1 ; Cl ; Cc1nn(COCC[Si](C)(C)C)c(C)c1-c1ccc(NC(=O)[C@@H](NC(=O)c2ccnn2CCCS(C)=O)C(C2CC2)C2CC2)cc1]

According to the method of Preparation 11 the compound of Preparation 344 (75 mg, 0.14 mmol) was reacted with the compound of Preparation 156 (34 mg, 0.14 mmol) to afford the title compound as an off-white solid (60 mg, 60% yield). 1H NMR (400 MHz, DMSO-d6) δ 10.24 (s, 1H), 8.52 (dd, J=8.61, 1.74 Hz, 1H), 7.67 (d, J=8.50 Hz, 2H), 7.53 (d, J=1.96 Hz, 1H), 7.22 (d, J=8.61 Hz, 2H), 7.06 (d, J=1.91 Hz, 1H), 5.34 (s, 2H), 4.83 (t, J=7.79 Hz, 1H), 4.59 (t, J=6.81 Hz, 2H), 3.58-3.53 (m, 2H), 2.47 (s, 3H), 2.25 (s, 3H) 2.13 (s, 3H), 2.09-2.01 (m, 2H), 1.30-1.17 (m, 2H), 0.75-0.50 (m, 5H), 0.50-0.10 (m, 8H), 0.02 (m, 9H); LCMS (METHOD 2) (ESI): m/z: 667 [M+H]⁺; 82%; RT=2.22 min, (ACQUITY BEH C18 column, 0.05% FA in water with MeCN).

### Preparation 367: tert-butyl N-[(1S)-2-[4-[3,5-dimethyl-1-(2-trimethylsilylethoxymethyl)pyrazol-4-yl]anilino]-1-(4-methylcyclohexyl)-2-oxo-ethyl]carbamate

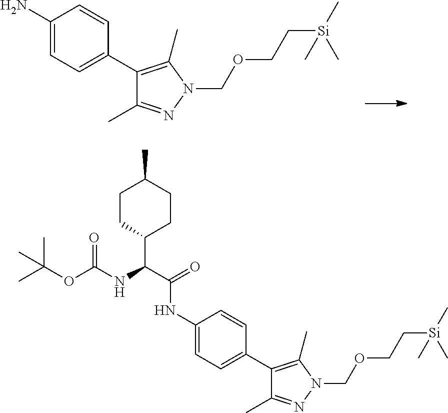

According to the method of Preparation 343 the compound of Preparation 342 (640 mg, 0.2 mmol) was reacted with (S)-2-((tert-butoxycarbonyl)amino)-2-((1r,4S)-4-methylcyclohexyl)acetic acid (synthesis described in WO2018229079, 500 mg, 1.84 mmol) to afford the title compound as an off-white solid (700 mg, 66% yield). 1H NMR (400 MHz, CDCl₃) δ 7.94 (s, 1H), 7.58-7.55 (m, 2H), 7.20 (d, J=8.5 Hz, 2H), 5.38 (s, 2H), 5.17-5.05 (m, 1H), 4.00 (dd, J=7.0, 8.3 Hz, 1H), 3.62 (dd, J=7.7, 8.8 Hz, 2H), 2.29 (s, 3H), 2.22 (s, 3H), 1.89-1.69 (m, 5H), 1.46 (s, 9H), 1.37-1.28 (m, 1H), 1.20-0.83 (m, 9H), 0.01-0.03 (m, 9H); LCMS (METHOD 2) (ESI): m/z: 571 [M+H]⁺; 97%; RT=2.72 min (ACQUITY BEH C18 column, 0.05% FA in water with MeCN).

### Preparation 368: (2S)-2-amino-N-[4-[3,5-dimethyl-1-(2-trimethylsilylethoxymethyl)pyrazol-4-yl]phenyl]-2-(4-methylcyclohexyl)acetamide hydrochloride

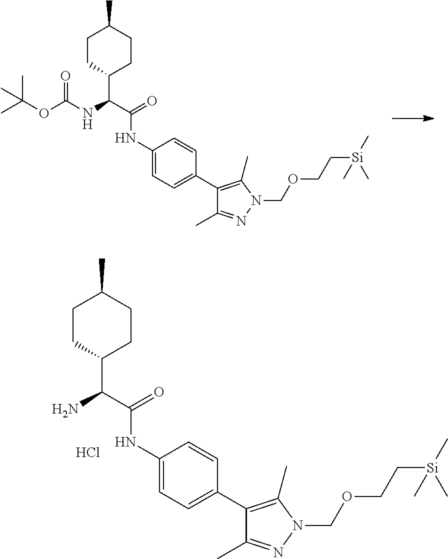 [SMILES: Cc1nn(COCC[Si](C)(C)C)c(C)c1-c1ccc(NC(=O)[C@@H](NC(=O)OC(C)(C)C)[C@H]2CC[C@H](C)CC2)cc1 ; Cc1nn(COCC[Si](C)(C)C)c(C)c1-c1ccc(NC(=O)[C@@H](N)[C@H]2CC[C@H](C)CC2)cc1 ; Cl]

The compound of Preparation 344 (240 mg, 0.42 mmol) was dissolved in 1M hydrogen chloride in MeOH (50 mL) and the reaction was stirred at room temperature for 3 hours. The mixture was concentrated in vacuo to give the title compound (200 mg, assume 100% yield) as an off-white solid. LCMS (METHOD 2) (ESI): m/z: 471 [M+H]⁺; 94%; RT=5.05 min (ACQUITY BEH C18 column, 0.05% FA in water with MeCN).

### Preparation 369: N-[(1S)-2-[4-[3,5-dimethyl-1-(2-trimethylsilylethoxymethyl)pyrazol-4-yl]anilino]-1-(4-methylcyclohexyl)-2-oxo-ethyl]-2-(2-methylsulfanylethyl)pyrazole-3-carboxamide

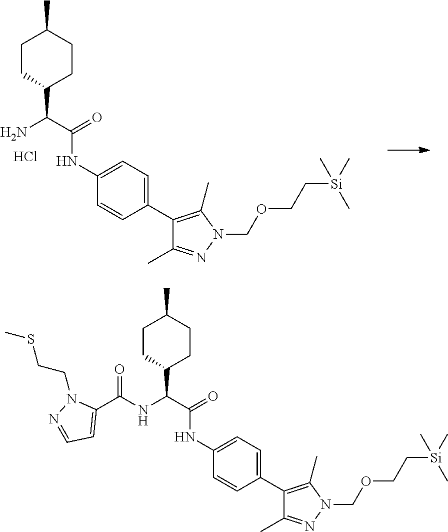

According to the method of Preparation 11 the compound of Preparation 368 (50.0 mg, 0.09 mmol) was reacted with the compound of Preparation 148 (18.4 mg, 0.09 mmol) to afford the title compound as an off-white solid (40 mg, 64% yield). 1H NMR (300 MHz, DMSO-d6) δ 10.24 (s, 1H), 8.57 (br d, J=8.1 Hz, 1H), 7.68 (br d, J=7.7 Hz, 2H), 7.51 (s, 1H), 7.20 (br d, J=7.7 Hz, 2H), 7.04 (s, 1H), 4.66 (br t, J=7.0 Hz, 2H), 4.40 (br t, J 10=8.3 Hz, 1H), 3.55 (br t, J=7.9 Hz, 2H), 2.79 (br t, J=6.8 Hz, 2H), 2.24 (s, 3H), 2.12 (s, 3H), 1.99 (s, 3H), 1.92-1.54 (m, 6H), 1.41-0.78 (m, 11H), 0.05-0.05 (m, 9H); LCMS (METHOD 2) (ESI): m/z: 639 [M+H]⁺; 93%; RT=2.61 min (ACQUITY BEH C18 column, 0.05% FA in water with MeCN).

### Preparation 370: N-[(1S)-2-[4-[3,5-dimethyl-1-(2-trimethylsilylethoxymethyl)pyrazol-4-yl]anilino]-1-(4-methylcyclohexyl)-2-oxo-ethyl]-2-(2-methylsulfinylethyl)pyrazole-3-carboxamide

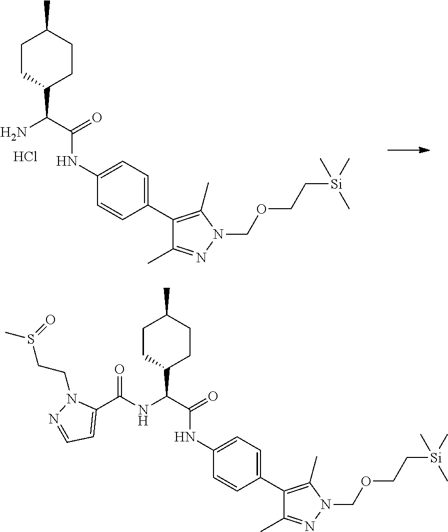

According to the method of Preparation 11 the compound of Preparation 368 (60 mg, 0.11 mmol) was reacted with the compound of Preparation 150 (23.5 mg, 0.19 mmol) to afford the title compound as an off-white solid (50 mg, 64% yield). 1H NMR (400 MHz, DMSO-d6) δ 10.25 (s, 1H), 8.61 (br d, J=8.1 Hz, 1H), 7.69 (br d, J=8.4 Hz, 2H), 7.55 (s, 1H), 7.21 (br d, J=8.4 Hz, 2H), 7.13 (s, 1H), 5.34 (s, 1H), 4.94-4.78 (m, 2H), 4.41 (br t, J=8.6 Hz, 1H), 3.56 (t, J=7.7 Hz, 2H), 3.25 (br d, J=7.7 Hz, 1H), 3.17-3.03 (m, 1H), 2.54 (s, 3H), 2.25 (s, 3H), 2.13 (s, 3H), 1.94-1.55 (m, 5H), 1.40-0.77 (m, 12H), 0.06-0.11 (m, 8H); LCMS (METHOD 2) (ESI): m/z: 653 [M−H]⁻; 93%; RT=2.34 min, (ACQUITY BEH C18 column, 0.05% FA in water with MeCN).

### Preparation 371: N-[(1S)-2-[4-[3,5-dimethyl-1-(2-trimethylsilylethoxymethyl)pyrazol-4-yl]anilino]-1-(4-methylcyclohexyl)-2-oxo-ethyl]-2-(3-methylsulfanylpropyl)pyrazole-3-carboxamide

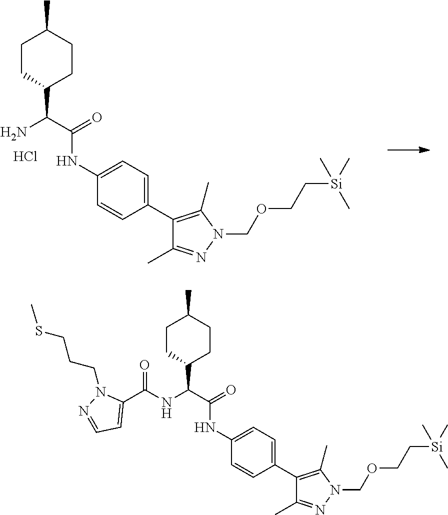

According to the method of Preparation 11 the compound of Preparation 368 (70.0 mg, 0.13 mmol) was reacted with the compound of Preparation 154 (26.0 mg, 0.09 mmol) to afford the title compound as an off-white solid (60 mg, 67% yield). LCMS (METHOD 2) (ESI): m/z: 653 [M+H]⁺; 92%; RT=2.65 min (ACQUITY BEH C18 column, 0.1% FA in water with MeCN).

### Preparation 372: N-[(1S)-2-[4-[3,5-dimethyl-1-(2-trimethylsilylethoxymethyl)pyrazol-4-yl]anilino]-1-(4-methylcyclohexyl)-2-oxo-ethyl]-2-(3-methylsulfinylpropyl)pyrazole-3-carboxamide

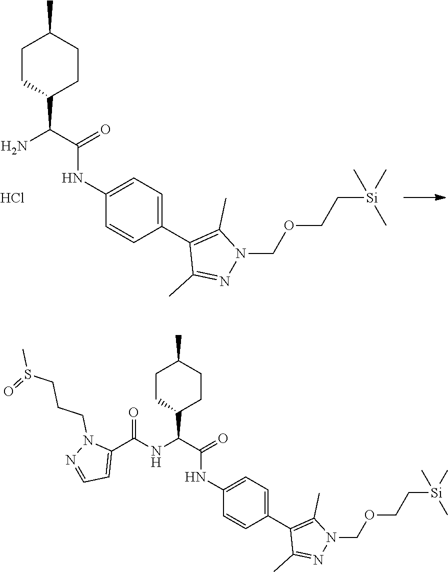 [SMILES: Cl ; Cc1nn(COCC[Si](C)(C)C)c(C)c1-c1ccc(NC(=O)[C@@H](N)[C@H]2CC[C@H](C)CC2)cc1 ; Cc1nn(COCC[Si](C)(C)C)c(C)c1-c1ccc(NC(=O)[C@@H](NC(=O)c2ccnn2CCCS(C)=O)[C@H]2CC[C@H](C)CC2)cc1]

According to the method of Preparation 11 the compound of Preparation 368 (100 mg, 0.20 mmol) was reacted with the compound of Preparation 156 (43 mg, 0.20 mmol) to afford the title compound as a yellow solid (60 mg, 75% yield). 1H NMR (300 MHz, DMSO-d6) δ 10.25 (s, 1H), 8.58 (d, J=7.34 Hz, 1H), 7.69 (d, J=8.44 Hz, 2H), 7.52 (d, J=2.20 Hz, 1H), 7.21 (d, J=8.44 Hz, 2H), 7.07 (d, J=2.20 Hz, 1H), 5.34 (s, 2H), 4.60-4.55 (m, 2H), 4.38 (br t, J=8.46 Hz, 1H), 3.55 (br t, J=7.89 Hz, 2H), 2.64-2.54 (m, 2H), 2.46 (d, J=3.81 Hz, 2H), 2.37 (s, 3H), 2.17 (s, 3H), 2.12-2.08 (m, 1H), 1.98 (s, 2H), 1.92-1.53 (m, 4H), 1.36-1.13 (m, 3H), 1.09-0.77 (m, 8H), 0.03 (s, 9H); LCMS (METHOD 2) (ESI): m/z: 669 [M+H]⁺; 91%; RT=2.62 min, (ACQUITY BEH C18 column, 0.05% FA in water with CAN).

### Example 373: 4-methylpent-4-enenitrile

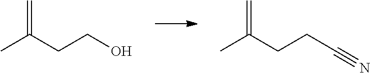

Mesyl chloride (22.0 mL, 284 mmol) was added dropwise to a solution of 3-methylbut-3-en-1-ol (20.0 g, 232.2 mmol) and triethylamine (50 mL, 358 mmol) in DCM (200 mL) at 5° C. over 20 minutes, The reaction mixture was stirred at room temperature for 4 hours then poured into H₂O (200 mL). The aqueous phase was collected and washed with DCM (50 mL). The combined organic phases were washed successively with 1M Hydrogen chloride (aq, 30 mL) and saturated NaHCO₃ (aq, 30 mL) then dried over MgSO₄, filtered and concentrated in vacuo to leave intermediate compound, 3-methylbut-3-enyl methanesulfonate (38.4 g, 100% yield). This intermediate was dissolved in DMSO (200 mL) and KCN (20.0 g, 307 mmol) was added and the reaction was stirred at 80° C. for 16 hours. To the cooled reaction mixture was added H₂O (300 mL) and the mixture was extracted with TBME (3×150 mL). The combined organic phases were dried over MgSO₄, filtered and concentrated in vacuo to leave the title compound as a brown oil. (19.2 g, 87% yield). 1H NMR (400 MHz, CDCl₃) δ 4.94-4.85 (m, 1H), 4.85-4.74 (m, 1H), 2.48 (dd, J=7.7, 6.6 Hz, 2H), 2.36 (t, J=7.4 Hz, 2H), 1.77 (d, J=1.4 Hz, 3H).

### Preparation 374: 4-methylpent-4-enal

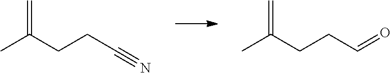

DIBAL (38 g, 66.8 mmol) in toluene was added to a solution of the compound of Preparation 373 (6.0 g, 63.1 mmol) in DCM (30 mL) at −78° C. The reaction mixture was warmed to 0° C. and stirred for 2 hours. The reaction mixture was carefully quenched with 2M aq. hydrogen chloride (250 mL). After phase separation, the aqueous phase was rewashed with TBME (3×100 mL) and the combined organic phase was filtered through silica gel, washing with TBME, then dried over MgSO₄, filtered and concentrated in vacuo to leave the crude title compound as a yellow oil that was used directly in the next step. (2.35 g, 38% yield).

### Preparation 375: ethyl (25,35)-3-formyl-2-(4-methoxyanilino)-5-methyl-hex-5-enoate

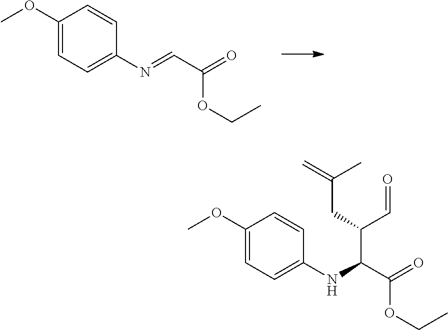 [SMILES: CCOC(=O)/C=N/c1ccc(OC)cc1 ; C=C(C)C[C@H](C=O)[C@H](Nc1ccc(OC)cc1)C(=O)OCC]

The compound of Preparation 374 (2.35 g, 4.89 mmol) was added to a stirring mixture of ethyl-2-(4-methoxyphenyl)iminoacetate (as described in Preparation 282, 3.0 g, 14.48 mmol) and (2S)-pyrrolidine-2-carboxylic acid (330 mg, 2.87 mmol) in DMF (20 mL) at room temperature. The reaction mixture was stirred for 3 hours, then diluted with TBME (75 mL) and the mixture was washed with H₂O (50 mL). The isolated aqueous phase was extracted with TBME (2×50 mL). The combined extracts were dried over Na₂SO₄, filtered, concentrated in vacuo and the obtained crude compound was purified by silica column chromatography (230-400 mesh), eluting with EtOAc in heptane, to afford the title compound as a yellow oil (3.7 g, 84% yield). 1H NMR (400 MHz, CDCl₃) δ 9.70 (d, J=1.7 Hz, 1H), 6.85-6.70 (m, 2H), 6.70-6.59 (m, 2H), 4.84 (dt, J=36.6, 1.5 Hz, 3H), 4.40-4.28 (m, 1H), 4.23-4.07 (m, 3H), 4.03 (s, 1H), 3.74 (s, 3H), 2.94 (dddd, J=8.8, 6.0, 4.5, 1.7 Hz, 1H), 2.71-2.52 (m, 1H), 2.37 (dd, J=14.7, 5.8 Hz, 1H), 1.80-1.68 (m, 5H), 1.24 (t, J=7.1 Hz, 4H). LCMS (METHOD 3) (ES): m/z 306.2 [M+H]⁺, RT=0.81 min.

### Preparation 376: ethyl (2S,3R)-2-(4-methoxyanilino)-5-methyl-3-vinyl-hex-5-enoate

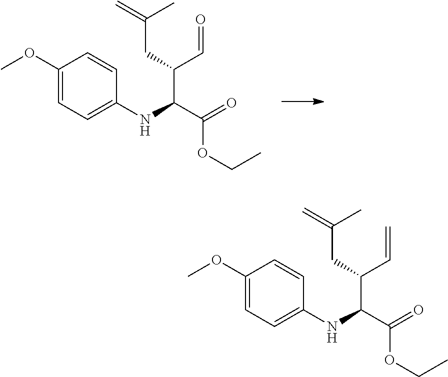 [SMILES: C=C(C)C[C@H](C=O)[C@H](Nc1ccc(OC)cc1)C(=O)OCC ; C=C[C@@H](CC(=C)C)[C@H](Nc1ccc(OC)cc1)C(=O)OCC]

p-Chloro[di(cyclopenta-2,4-dien-1-yl)]dimethyl(p-methylene)titaniumaluminum (0.5 M in toluene, 40 mL, 20 mmol) was added to a solution of the compound of Preparation 375 (2.0 g, 6.56 mmol) in THF (20 mL) at −78° C. and stirred for 1 hour, then warmed to room temperature and stirred for 2 hours. The reaction mixture was then added to a mixture of ice water (100 g) and 5M NaOH (20 mL) with stirring. The mixture was filtered through Celite, washing with TBME (5×30 mL). The phases were separated and the organic phase was dried over Na₂SO₄, filtered, and the obtained crude compound was purified by silica column chromatography (230-400 mesh), eluting with EtOAc in heptane, to afford the title compound (0.37 g, 19% yield). 1H NMR (600 MHz, CDCl₃) δ 6.83-6.69 (m, 2H), 6.69-6.53 (m, 2H), 5.70-5.51 (m, 1H), 5.19-5.09 (m, 2H), 4.87-4.64 (m, 3H), 4.25-4.08 (m, 2H), 4.08-3.92 (m, 1H), 3.73 (s, 3H), 2.66 (tt, J=9.4, 4.8 Hz, 1H), 2.47-2.30 (m, 1H), 2.17-2.03 (m, 1H), 1.75-1.65 (m, 3H), 1.29-1.17 (m, 3H).

### Preparation 377: ethyl (2S,3R)-2-(benzyloxycarbonylamino)-5-methyl-3-vinyl-hex-5-enoate

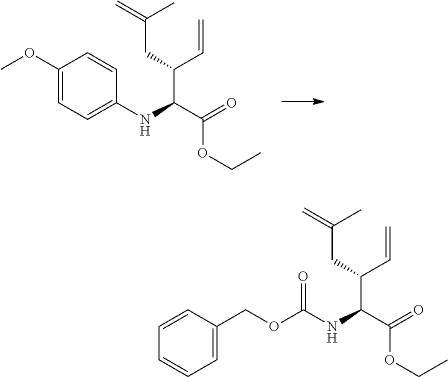 [SMILES: C=C[C@@H](CC(=C)C)[C@H](Nc1ccc(OC)cc1)C(=O)OCC ; C=C[C@@H](CC(=C)C)[C@H](NC(=O)OCc1ccccc1)C(=O)OCC]

According to the method of Preparation 284 the compound of Preparation 376 (347 mg, 1.14 mmol) was reacted to afford the title compound as a yellow oil (229 mg, 60% yield).

1H NMR (600 MHz, CDCl₃) δ 7.41-7.28 (m, 5H), 5.54 (dt, J=17.0, 9.7 Hz, 1H), 5.35 (d, J=8.9 Hz, 1H), 5.20-5.03 (m, 4H), 4.77 (d, J=40.9 Hz, 2H), 4.42 (dd, J=9.0, 4.9 Hz, 1H), 4.30-4.08 (m, 3H), 2.66 (hept, J=5.0 Hz, 1H), 2.29 (dd, J=14.1, 5.8 Hz, 1H), 2.10 (dd, J=14.1, 9.1 Hz, 1H), 1.71 (d, J=4.9 Hz, 3H), 1.29 (t, J=7.2 Hz, 3H).

### Preparation 378: ethyl (2S,3R)-2-(benzyloxycarbonylamino)-3-cyclopropyl-4-(1-methylcyclopropyl)butanoate

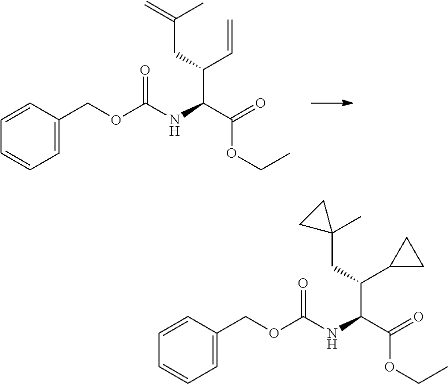 [SMILES: C=C[C@@H](CC(=C)C)[C@H](NC(=O)OCc1ccccc1)C(=O)OCC ; CCOC(=O)[C@@H](NC(=O)OCc1ccccc1)[C@H](CC1(C)CC1)C1CC1]

According to the method of Preparation 285 the compound of Preparation 377 (100 mg, 0.30 mmol) was reacted to afford the title compound as a colourless oil (69 mg, 47% yield). The material contained around 25% starting olefin. Taken on without further purification.

### Preparation 379: (2S,3R)-2-(benzyloxycarbonylamino)-3-cyclopropyl-4-(1-methylcyclopropyl)butanoic acid

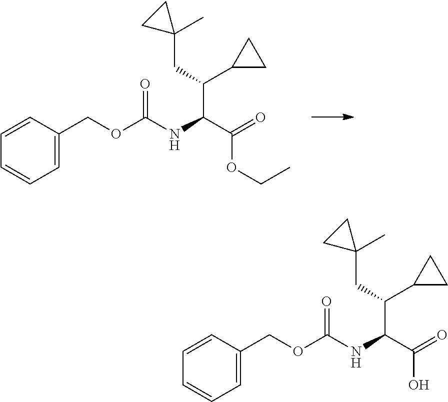 [SMILES: CCOC(=O)[C@@H](NC(=O)OCc1ccccc1)[C@H](CC1(C)CC1)C1CC1 ; CC1(C[C@H](C2CC2)[C@H](NC(=O)OCc2ccccc2)C(=O)O)CC1]

KOH (100 mg, 1.78 mmol) was added to a solution of the compound of Preparation 378 (69 mg, 0.14 mmol) in MeOH (7.5 mL) and H₂O (2.5 mL) at room temperature. The reaction mixture was stirred for 4 hours. The reaction mixture was diluted with H₂O (15 mL) and extracted with TBME (2×5 mL). The aqueous phase was collected and acidified to pH 1 with 5M aq. hydrogen chloride, then extracted with TBME (3×10 mL). The combined extracts were dried over MgSO₄, filtered and concentrated in vacuo to afford the title compound (44 mg, 69% yield). Used directly in the next step.

### Preparation 380: benzyl N-[(1S,2R)-2-cyclopropyl-1-[[4-[3,5-dimethyl-1-(2-trimethylsilylethoxymethyl)pyrazol-4-yl]phenyl]carbamoyl]-3-(1-methylcyclopropyl)propyl]carbamate

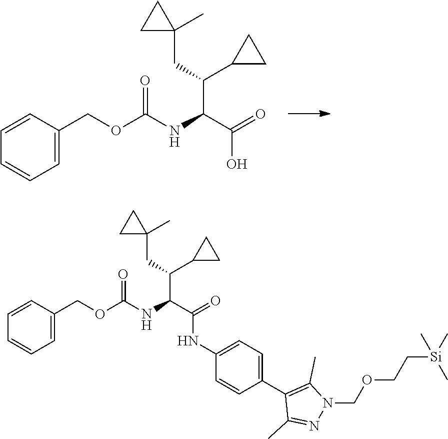 [SMILES: CC1(C[C@H](C2CC2)[C@H](NC(=O)OCc2ccccc2)C(=O)O)CC1 ; Cc1nn(COCC[Si](C)(C)C)c(C)c1-c1ccc(NC(=O)[C@@H](NC(=O)OCc2ccccc2)[C@H](CC2(C)CC2)C2CC2)cc1]

HATU (55.3 mg, 0.15 mmol) was added to a solution of the compound of Preparation 379 (44.0 mg, 0.097 mmol), the compound of Preparation 342 (60 mg, 0.19 mmol) and DIPEA (0.2 mL, 1.15 mmol) in dry MeCN (5 mL) and the mixture was stirred at room temperature for 4 hours. The reaction mixture was purified directly via prep. acidic HPLC to give the title compound as a colourless solid (55 mg, 90% yield); LCMS (METHOD 3) (ES): m/z 631.4 [M+H]⁺, RT=1.06 min.

### Preparation 381: (2S,3R)-2-amino-3-cyclopropyl-N-[4-[3,5-dimethyl-1-(2-trimethylsilylethoxymethyl)pyrazol-4-yl]phenyl]-4-(1-methylcyclopropyl)butanamide

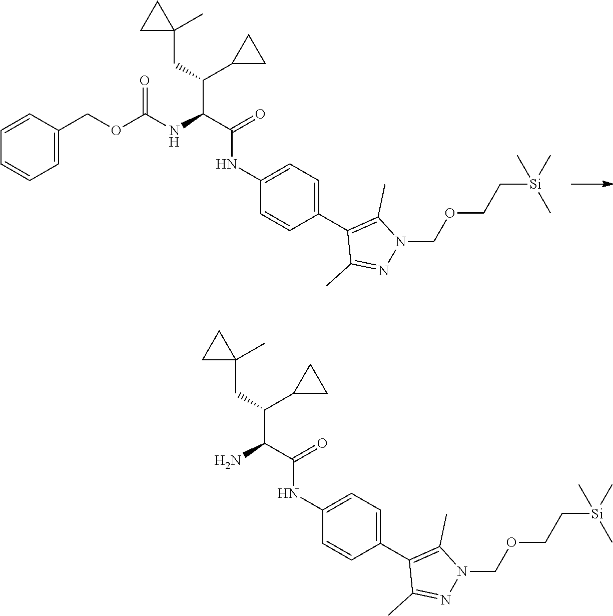 [SMILES: Cc1nn(COCC[Si](C)(C)C)c(C)c1-c1ccc(NC(=O)[C@@H](NC(=O)OCc2ccccc2)[C@H](CC2(C)CC2)C2CC2)cc1 ; Cc1nn(COCC[Si](C)(C)C)c(C)c1-c1ccc(NC(=O)[C@@H](N)[C@H](CC2(C)CC2)C2CC2)cc1]

Triethylsilane (0.1 mL) was added to a mixture of the compound of Preparation 380 (20.0 mg, 0.032 mmol) and Pd/C (10%, 5 mg, 0.005 mmol) in MeOH (3 mL) and the reaction mixture was stirred for 2 hours, then concentrated in vacuo to afford the crude title compound that was used directly in the next step. (15.7 mg, assume 100% yield); LCMS (METHOD 3) (ES): m/z 497.3 [M+H]⁺, RT=0.77 min.

### Preparation 382: N-[(1S,2R)-2-cyclopropyl-1-[[4-[3,5-dimethyl-1-(2-trimethylsilylethoxy-methyl)pyrazol-4-yl]phenyl]carbamoyl]-3-(1-methylcyclopropyl)propyl]-2-ethyl-pyrazole-3-carboxamide

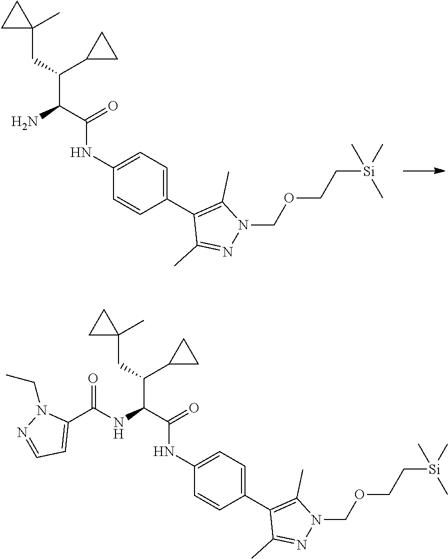

According to the method of Preparation 11 the compound of Preparation 381 (8.0 mg, 0.016 mmol) was reacted with 2-ethylpyrazole-3-carboxylic acid (4.0 mg, 0.028 mmol) to afford the title compound, after prep. acidic HPLC, as an off-white solid (4 mg, 40% yield). LCMS (METHOD 3) (ES): m/z 619.4 [M+H]⁺, RT=1.02 min.

### Preparation 383: (2R)-4-methylpent-4-en-2-ol

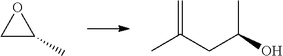

(2R)-2-methyloxirane (1.5 g, 26.0 mmol) was added to a stirring suspension of copper iodide (1.5 g, 7.7 mmol) in THF (10 mL) at −78° C. The suspension was stirred for 10 minutes then bromo(isopropenyl)magnesium (0.5 M solution in THF, 77.0 mL, 39 mmol) was added dropwise. On complete addition the reaction mixture was warmed to room temperature and stirred for 2 hours. The reaction mixture was quenched into saturated NH₄Cl (aq. soln 50 mL) and diluted with H₂O (50 mL). The mixture was extracted with Et₂O (2×100 mL). The combined organic phase was dried over MgSO₄, filtered, concentrated in vacuo and the obtained crude compound was purified by silica column chromatography (230-400 mesh), eluting with DCM, to afford the title compound (2.0 g, 77% yield). 1H NMR (600 MHz, CDCl₃) δ 4.84 (dt, J=49.0, 2.1 Hz, 2H), 3.93 (dddd, J=8.3, 6.3, 4.3, 2.0 Hz, 1H), 2.23-2.07 (m, 2H), 1.76 (d, J=1.6 Hz, 3H), 1.26-1.13 (m, 3H).

### Preparation 384: [(1R)-1,3-dimethylbut-3-enyl] 4-methylbenzenesulfonate

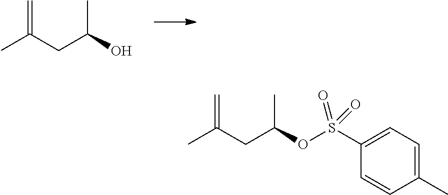

Tosyl chloride (2.2 g, 12.0 mmol) was added to a solution of the compound of Preparation 383 (2.0 g, 20 mmol) and DABCO (2.0 g, 17.8 mmol) in DCM (30 mL) and stirred at room temperature for 16 hours. The reaction mixture was washed with H₂O (50 mL). The aqueous phase was extracted with DCM (30 mL) then the combined organic phase was dried over MgSO₄, filtered and concentrated in vacuo, and the obtained crude compound was purified by silica column chromatography (230-400 mesh), eluting with DCM, to afford the title compound as an off-white solid (2.8 g, 55% yield). 1H NMR (600 MHz, CDCl₃) δ 7.86-7.72 (m, 2H), 7.32 (d, J=8.0 Hz, 2H), 4.77-4.71 (m, 2H), 4.68 (h, J=1.0 Hz, 1H), 2.45 (s, 3H), 2.27 (dddd, J=104.4, 13.9, 6.7, 1.1 Hz, 2H), 1.59 (t, J=1.2 Hz, 3H), 1.27 (d, J=6.3 Hz, 3H).

### Preparation 385: ethyl (2S,3S)-2-(benzhydrylideneamino)-3,5-dimethyl-hex-5-enoate

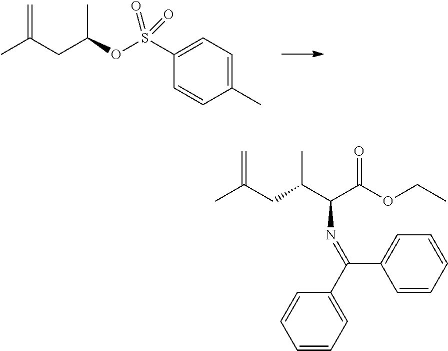 [SMILES: C=C(C)C[C@@H](C)OS(=O)(=O)c1ccc(C)cc1 ; C=C(C)C[C@H](C)[C@H](N=C(c1ccccc1)c1ccccc1)C(=O)OCC]

LiHMDS (1.0 M solution in THF, 6.0 mL) was added to a solution of the compound of Preparation 384 (1.0 g, 3.93 mmol9 and ethyl 2-(benzhydrylideneamino)acetate (1.2 g, 4.5 mmol) in THF (5 mL) at 5° C. On complete addition the reaction mixture was stirred at 90° C. for 16 hours. The cooled reaction mixture was diluted with Et₂O and H₂O (25 mL each) and the phases were separated. The aqueous phase was extracted with Et₂O (10 mL). The combined organic phase was dried over MgSO₄, filtered and concentrated in vacuo, and the obtained crude compound was purified by silica column chromatography (230-400 mesh), eluting with EtOAc in heptane, to afford the title compound (as a mixture of diastereomers), as a yellow oil (490 mg, 35% yield). LCMS (METHOD 3) (ES): m/z 350.3 [M+H]⁺, RT=1.06 min.

### Preparation 386: ethyl (2S,3S)-2-(benzyloxycarbonylamino)-3,5-dimethyl-hex-5-enoate

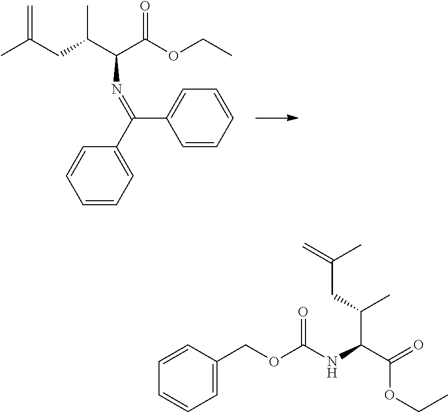 [SMILES: C=C(C)C[C@H](C)[C@H](N=C(c1ccccc1)c1ccccc1)C(=O)OCC ; C=C(C)C[C@H](C)[C@H](NC(=O)OCc1ccccc1)C(=O)OCC]

Hydrogen chloride (2M aq. solution, 5 mL) was added to a solution of the compound of Preparation 385 (790 mg, 2.26 mmol) in THF (10 mL) and stirred at room temperature for 20 minutes. The reaction mixture was diluted with H₂O (20 mL) and extracted with Et₂O (20 mL). The aqueous phase was diluted with THF (10 mL) and basified to pH 8 with saturated Na₂CO₃ (aq.soln). Benzyl carbonochloridate (600 mg, 3.52 mmol) was added and the reaction mixture was stirred at room temperature for 1 hour. The reaction mixture was diluted with H₂O (10 mL) and extracted with Et₂O (2×20 mL). The combined organic phase was dried over MgSO₄, filtered and concentrated in vacuo, and the obtained crude compound was purified by silica column chromatography (230-400 mesh), eluting with EtOAc in heptane, to afford the title compound (as a mixture of diastereomers), as a colourless oil (518 mg, 71% yield). 1H NMR (600 MHz, CDCl₃) δ 7.40-7.27 (m, 5H), 5.29 (dd, J=71.2, 9.1 Hz, 1H), 5.12 (t, J=2.6 Hz, 2H), 4.80 (d, J=8.6 Hz, 1H), 4.72 (s, 1H), 4.39 (ddd, J=28.2, 9.0, 4.0 Hz, 1H), 4.27-4.15 (m, 2H), 2.37-2.08 (m, 2H), 1.94-1.81 (m, 1H), 1.71 (d, J=3.3 Hz, 3H), 1.28 (q, J=7.0 Hz, 4H), 0.86 (dd, J=46.1, 6.9 Hz, 3H).

### Preparation 387: ethyl (2S,3S)-2-(benzyloxycarbonylamino)-3-methyl-4-(1-methylcyclopropyl)butanoate

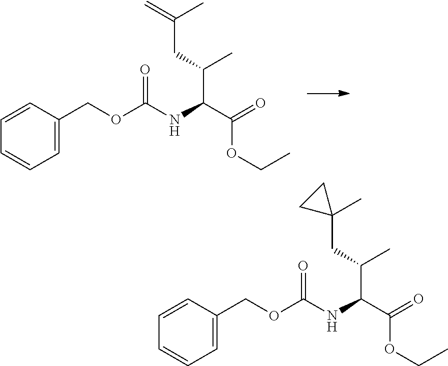

According to the method of Preparation 285 the compound of Preparation 386 (100 mg, 0.30 mmol) was reacted to afford the title compound (as a mixture of diastereomers), as a colourless oil (151 mg, 78% yield). 1H NMR (600 MHz, CDCl₃) δ 7.40-7.28 (m, 5H), 5.25 (dd, J=76.4, 9.1 Hz, 1H), 5.11 (s, 2H), 4.42 (ddd, J=26.9, 9.1, 3.9 Hz, 1H), 4.32-4.07 (m, 2H), 2.43-2.14 (m, 1H), 1.55 (ddd, J=28.4, 14.0, 5.3 Hz, 1H), 1.36-1.23 (m, 3H), 1.01 (dd, J=17.3, 8.5 Hz, 5H), 0.89 (d, J=6.9 Hz, 2H), 0.82 (dd, J=13.8, 10.0 Hz, 1H), 0.33-0.19 (m, 3H).

### Preparation 388: benzyl N-[(1S,2S)-1-[[4-[3,5-dimethyl-1-(2-trimethylsilylethoxymethyl)-pyrazol-4-yl]phenyl]carbamoyl]-2-methyl-3-(1-methylcyclopropyl)propyl]carbamate

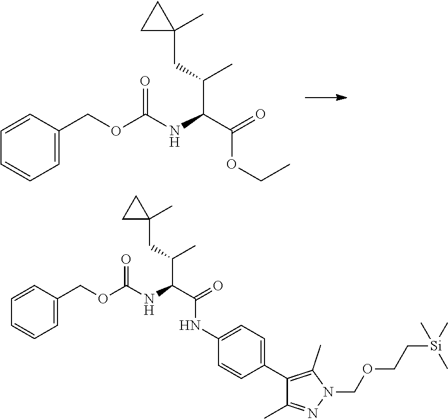 [SMILES: CCOC(=O)[C@@H](NC(=O)OCc1ccccc1)[C@@H](C)CC1(C)CC1 ; Cc1nn(COCC[Si](C)(C)C)c(C)c1-c1ccc(NC(=O)[C@@H](NC(=O)OCc2ccccc2)[C@@H](C)CC2(C)CC2)cc1]

Tert-butylmagnesium chloride (1M solution in THF, 2.0 mL, 2.0 mmol) was added to a solution of the compound of Preparation 387 (151 mg, 0.45 mmol) and the compound of Preparation 342 (150 mg, 0.47 mmol) in THF (4 mL) at room temperature and stirred for 3 hours. The reaction mixture was quenched with saturated NH₄Cl (aq. soln, 5 mL) and diluted with H₂O (10 mL). The mixture was extracted with DCM (2×15 mL). The organic phase was concentrated in vacuo and the residue was dissolved in MeCN and purified by prep. acidic HPLC, to afford the title compound as a mixture of diastereomers. (211 mg, 77% yield). LCMS (METHOD 3) (ES): m/z 605.5 [M+H]⁺, RT=1.03 min.

### Preparation 389: (25,35)-2-amino-N-[4-[3,5-dimethyl-1-(2-trimethylsilylethoxymethyl)-pyrazol-4-yl]phenyl]-3-methyl-4-(1-methylcyclopropyl)butanamide

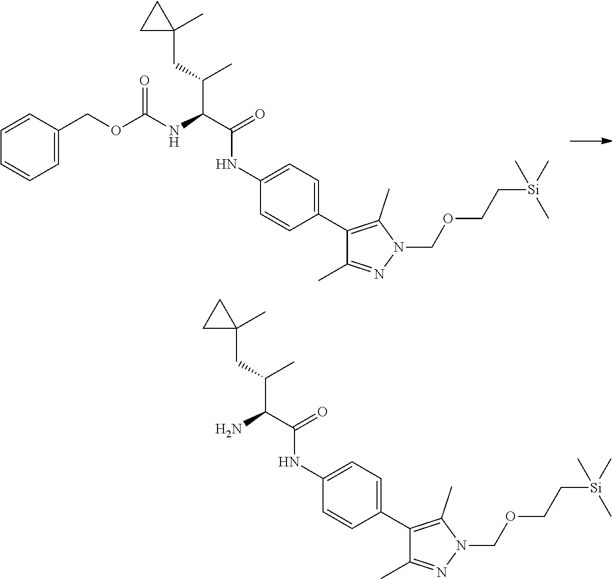 [SMILES: Cc1nn(COCC[Si](C)(C)C)c(C)c1-c1ccc(NC(=O)[C@@H](NC(=O)OCc2ccccc2)[C@@H](C)CC2(C)CC2)cc1 ; Cc1nn(COCC[Si](C)(C)C)c(C)c1-c1ccc(NC(=O)[C@@H](N)[C@@H](C)CC2(C)CC2)cc1]

According to the method of Preparation 381 the compound of Preparation 388 (214 mg, 0.35 mmol) was reacted to afford the title compound (as a mixture of diastereomers), as a colourless oil (166 mg, assume 100% yield). LCMS (METHOD 3) (ES): m/z 471.4 [M+H]⁺, RT=0.91 min.

### Preparation 390: N-[(1S,2S)-1-[[4-[3,5-dimethyl-1-(2-trimethylsilylethoxymethyl)pyrazol-4-yl]phenyl]carbamoyl]-2-methyl-3-(1-methylcyclopropyl)propyl]-2-ethyl-pyrazole-3-carboxamide

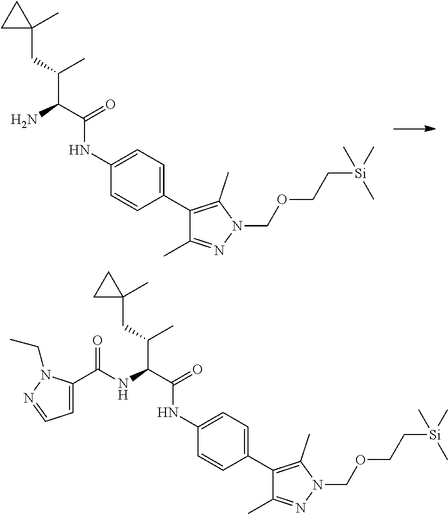

According to the method of Preparation 11 the compound of Preparation 389 (60 mg, 0.127 mmol) was reacted with 2-ethylpyrazole-3-carboxylic acid (24.0 mg, 0.171 mmol) to afford the title compound (as a mixture of diastereomers), after prep. acidic HPLC, as an off-white solid (54 mg, 71% yield). LCMS (METHOD 3) (ES): m/z 593.6 [M+H]⁺, RT=0.99 min.

### EXAMPLES

### Example 1: N-[(1S)-1-(dicyclopropylmethyl)-2-[[5-(3,5-dimethyl-1H-pyrazol-4-yl)-6-fluoro-2-pyridyl]amino]-2-oxo-ethyl]-2-isopropyl-pyrazole-3-carboxamide

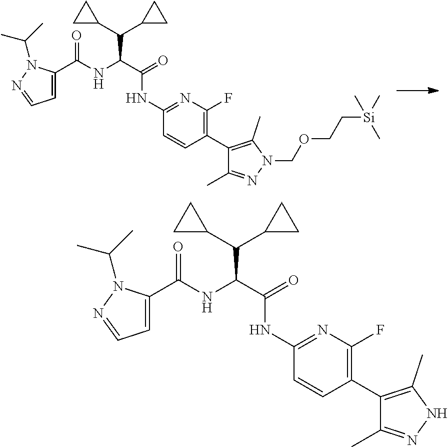

TFA (2 mL) was added to a solution of the compound of Preparation 42 (140 mg, 0.22 mmol) in DCM (2 mL) and stirred at room temperature for 2 hours. The reaction mixture was concentrated in vacuo and purified directly by prep. acidic HPLC to afford the title compound as an off-white solid (75 mg, 69% yield). ¹H NMR (600 MHz, DMSO-d6) δ 10.89 (s, 1H), 8.44 (d, J=8.6 Hz, 1H), 8.04 (dd, J=8.2, 1.8 Hz, 1H), 7.92-7.81 (m, 1H), 7.51 (d, J=2.0 Hz, 1H), 6.91 (d, J=2.0 Hz, 1H), 5.38 (hept, J=6.7 Hz, 1H), 4.89 (t, J=8.0 Hz, 1H), 2.11 (s, 6H), 1.36 (dd, J=23.2, 6.6 Hz, 6H), 0.96 (dq, J=8.3, 4.2, 3.3 Hz, 1H), 0.85 (qd, J=7.2, 4.3 Hz, 1H), 0.76 (td, J=9.5, 7.5 Hz, 1H), 0.52-0.45 (m, 1H), 0.39 (tdd, J=8.7, 5.5, 3.9 Hz, 1H), 0.35-0.25 (m, 2H), 0.25-0.19 (m, 3H), 0.17 (qd, J=7.1, 6.5, 2.1 Hz, 1H); LCMS (ES): m/z 494.268 [M+H]⁺; RT=2.34 min.

The examples listed in the table below were all accessed using the method described for Example 1.

| | Precursor | | | | |
|---|---|---|---|---|---|
| Ex. | Prep. | | | LCMS | |
| No. | number | Structure | Name | RT | Mass ion |
| 2 | 40 | 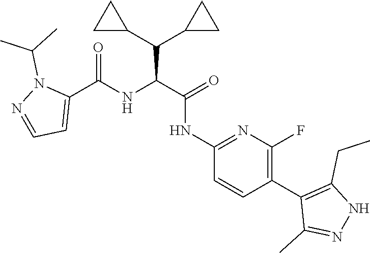 [SMILES: CCc1[nH]nc(C)c1-c1ccc(NC(=O)[C@@H](NC(=O)c2ccnn2C(C)C)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2- isopropyl-pyrazole-3- carboxamide | 2.39 | 508.284 |
| 3 | 45 | 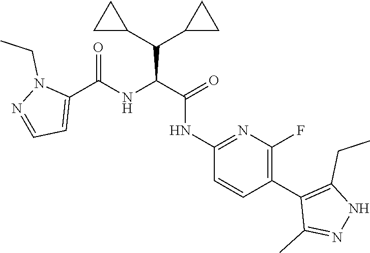 [SMILES: CCc1[nH]nc(C)c1-c1ccc(NC(=O)[C@@H](NC(=O)c2ccnn2CC)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-ethyl- pyrazole-3-carboxamide | 2.32 | 494.268 |
| 4 | 11 | 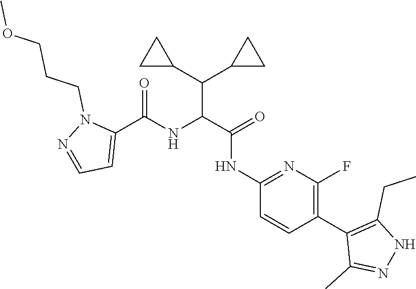 [SMILES: CCc1[nH]nc(C)c1-c1ccc(NC(=O)C(NC(=O)c2ccnn2CCCOC)C(C2CC2)C2CC2)nc1F] | N-[1- (dicyclopropylmethyl)-2- [[5-(5-ethyl-3-methyl- 1H-pyrazol-4-yl)-6- fluoro-2-pyridyl]amino]- 2-oxo-ethyl]-2-(3- methoxypropyl)pyrazole- 3-carboxamide | 2.24 | 520.304 |
| 5 | 12 | 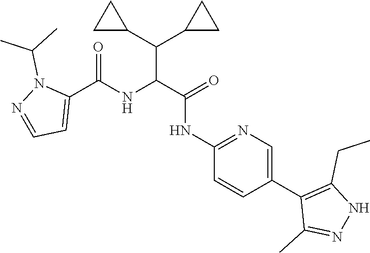 [SMILES: CCc1[nH]nc(C)c1-c1ccc(NC(=O)C(NC(=O)c2ccnn2C(C)C)C(C2CC2)C2CC2)nc1] | N-[1- (dicyclopropylmethyl)-2- [[5-(5-ethyl-3-methyl- 1H-pyrazol-4-yl)-2- pyridyl]amino]-2-oxo- ethyl]-2-isopropyl- pyrazole-3-carboxamide | 2.32 | 490.293 |
| 6 | 19 | 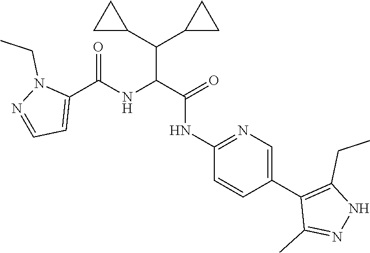 [SMILES: CCc1[nH]nc(C)c1-c1ccc(NC(=O)C(NC(=O)c2ccnn2CC)C(C2CC2)C2CC2)nc1] | N-[1- (dicyclopropylmethyl)-2- [[5-(5-ethyl-3-methyl- 1H-pyrazol-4-yl)-2- pyridyl]amino]-2-oxo- ethyl]-2-ethyl-pyrazole- 3-carboxamide | 2.24 | 476.278 |
| 7 | 22 | 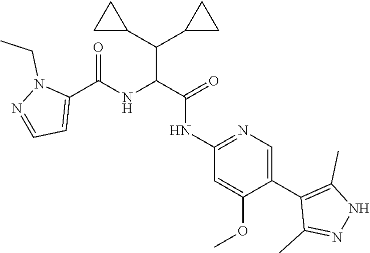 [SMILES: CCn1nccc1C(=O)NC(C(=O)Nc1cc(OC)c(-c2c(C)n[nH]c2C)cn1)C(C1CC1)C1CC1] | N-[1- (dicyclopropylmethyl)-2- [[5-(3,5-dimethyl-1H- pyrazol-4-yl)-4- methoxy-2- pyridyl]amino]-2-oxo- ethyl]-2-ethyl-pyrazole- 3-carboxamide | 2.12 | 492.272 |
| 8 | 13 | 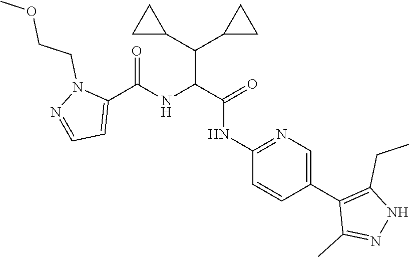 [SMILES: CCc1[nH]nc(C)c1-c1ccc(NC(=O)C(NC(=O)c2ccnn2CCOC)C(C2CC2)C2CC2)nc1] | N-[1- (dicyclopropylmethyl)-2- [[5-(5-ethyl-3-methyl- 1H-pyrazol-4-yl)-2- pyridyl]amino]-2-oxo- ethyl]-2-(2- methoxyethyl)pyrazole- 3-carboxamide | 2.20 | 506.288 |
| 9 | 24 | 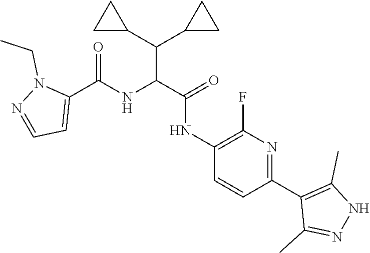 [SMILES: CCn1nccc1C(=O)NC(C(=O)Nc1ccc(-c2c(C)n[nH]c2C)nc1F)C(C1CC1)C1CC1] | N-[1- (dicyclopropylmethyl)-2- [[6-(3,5-dimethyl-1H- pyrazol-4-yl)-2-fluoro-3- pyridyl]amino]-2-oxo- ethyl]-2-ethyl-pyrazole- 3-carboxamide | 2.24 | 480.253 |
| 10 | 26 | 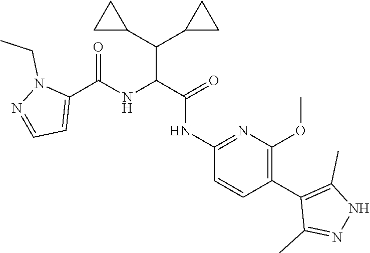 [SMILES: CCn1nccc1C(=O)NC(C(=O)Nc1ccc(-c2c(C)n[nH]c2C)c(OC)n1)C(C1CC1)C1CC1] | N-[1- (dicyclopropylmethyl)-2- [[5-(3,5-dimethyl-1H- pyrazol-4-yl)-6- methoxy-2- pyridyl]amino]-2-oxo- ethyl]-2-ethyl-pyrazole- 3-carboxamide | 2.26 | 492.272 |
| 11 | 48 | 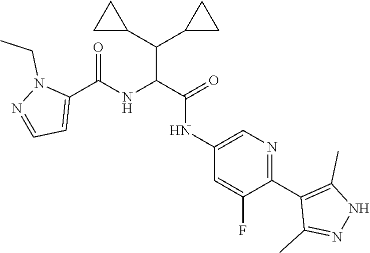 [SMILES: CCn1nccc1C(=O)NC(C(=O)Nc1cnc(-c2c(C)n[nH]c2C)c(F)c1)C(C1CC1)C1CC1] | N-[1- (dicyclopropylmethyl)-2- [[6-(3,5-dimethyl-1H- pyrazol-4-yl)-5-fluoro-3- pyridyl]amino]-2-oxo- ethyl]-2-ethyl-pyrazole- 3-carboxamide | 2.18 | 480.252 |
| 12 | 28 | 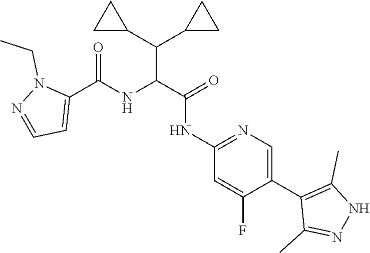 [SMILES: CCn1nccc1C(=O)NC(C(=O)Nc1cc(F)c(-c2c(C)n[nH]c2C)cn1)C(C1CC1)C1CC1] | N-[1- (dicyclopropylmethyl)-2- [[5-(3,5-dimethyl-1H- pyrazol-4-yl)-4-fluoro-2- pyridyl]amino]-2-oxo- ethyl]-2-ethyl-pyrazole- 3-carboxamide | 2.25 | 480.252 |
| 13 | 30 | 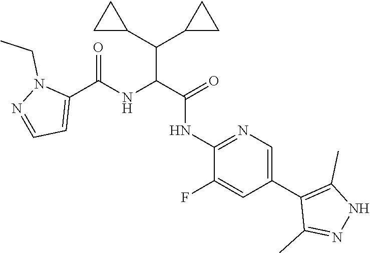 [SMILES: CCn1nccc1C(=O)NC(C(=O)Nc1ncc(-c2c(C)n[nH]c2C)cc1F)C(C1CC1)C1CC1] | N-[1- (dicyclopropylmethyl)-2- [[5-(3,5-dimethyl-1H- pyrazol-4-yl)-3-fluoro-2- pyridyl]amino]-2-oxo- ethyl]-2-ethyl-pyrazole- 3-carboxamide | 2.14 | 480.252 |
| 14 | 32 | 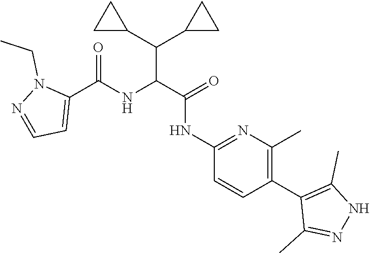 [SMILES: CCn1nccc1C(=O)NC(C(=O)Nc1ccc(-c2c(C)n[nH]c2C)c(C)n1)C(C1CC1)C1CC1] | N-[1- (dicyclopropylmethyl)-2- [[5-(3,5-dimethyl-1H- pyrazol-4-yl)-6-methyl- 2-pyridyl]amino]-2-oxo- ethyl]-2-ethyl-pyrazole- 3-carboxamide | 2.20 | 476.277 |
| 15 | 34 | 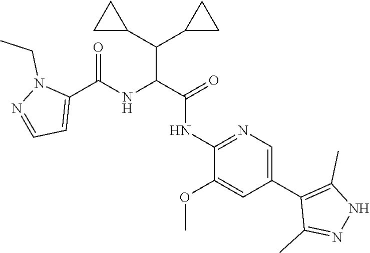 [SMILES: CCn1nccc1C(=O)NC(C(=O)Nc1ncc(-c2c(C)n[nH]c2C)cc1OC)C(C1CC1)C1CC1] | N-[1- (dicyclopropylmethyl)-2- [[5-(3,5-dimethyl-1H- pyrazol-4-yl)-3- methoxy-2- pyridyl]amino]-2-oxo- ethyl]-2-ethyl-pyrazole- 3-carboxamide | 2.11 | 492.272 |
| 16 | 76 | 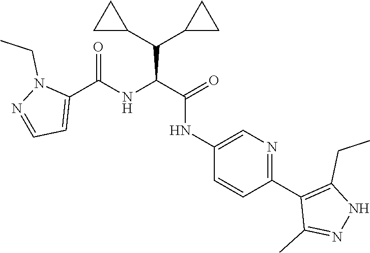 [SMILES: CCc1[nH]nc(C)c1-c1ccc(NC(=O)[C@@H](NC(=O)c2ccnn2CC)C(C2CC2)C2CC2)cn1] | N-[(1S)-1- (dicyclopropylmethyl)-2- [[6-(5-ethyl-3-methyl- 1H-pyrazol-4-yl)-3- pyridyl]amino]-2-oxo- ethyl]-2-ethyl-pyrazole- 3-carboxamide | 2.13 | 476.277 |
| 17 | 77 | 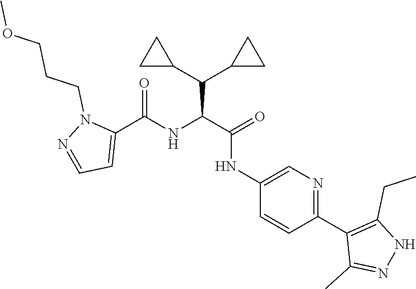 [SMILES: CCc1[nH]nc(C)c1-c1ccc(NC(=O)[C@@H](NC(=O)c2ccnn2CCCOC)C(C2CC2)C2CC2)cn1] | N-[(1S)-1- (dicyclopropylmethyl)-2- [[6-(5-ethyl-3-methyl- 1H-pyrazol-4-yl)-3- pyridyl]amino]-2-oxo- ethyl]-2-(3- methoxypropyl)pyrazole- 3-carboxamide | 2.13 | 520.304 |
| 18 | 78 | 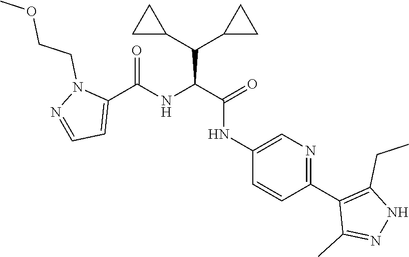 [SMILES: CCc1[nH]nc(C)c1-c1ccc(NC(=O)[C@@H](NC(=O)c2ccnn2CCOC)C(C2CC2)C2CC2)cn1] | N-[(1S)-1- (dicyclopropylmethyl)-2- [[6-(5-ethyl-3-methyl- 1H-pyrazol-4-yl)-3- pyridyl]amino]-2-oxo- ethyl]-2-(2- methoxyethyl)pyrazole- 3-carboxamide | 2.09 | 506.288 |
| 19 | 79 | 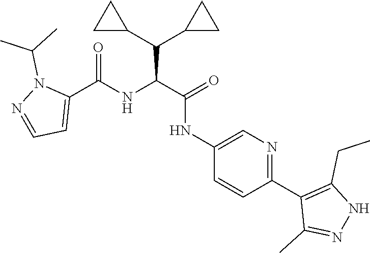 [SMILES: CCc1[nH]nc(C)c1-c1ccc(NC(=O)[C@@H](NC(=O)c2ccnn2C(C)C)C(C2CC2)C2CC2)cn1] | N-[(1S)-1- (dicyclopropylmethyl)-2- [[6-(5-ethyl-3-methyl- 1H-pyrazol-4-yl)-3- pyridyl]amino]-2-oxo- ethyl]-2-isopropyl- pyrazole-3-carboxamide | 2.20 | 490.293 |
| 20 | 81 | 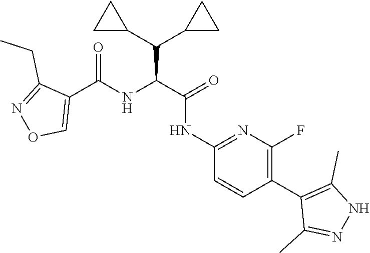 [SMILES: CCc1nocc1C(=O)N[C@H](C(=O)Nc1ccc(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-6-fluoro-2- pyridyl]amino]-2-oxo- ethyl]-3-ethyl-isoxazole- 4-carboxamide | 2.31 | 481.236 |
| 21 | 54 | 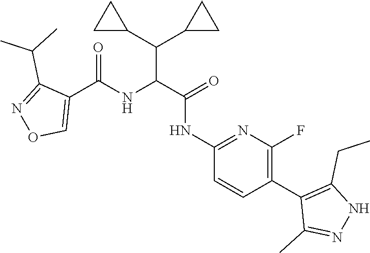 [SMILES: CCc1[nH]nc(C)c1-c1ccc(NC(=O)C(NC(=O)c2conc2C(C)C)C(C2CC2)C2CC2)nc1F] | N-[1- (dicyclopropylmethyl)-2- [[5-(5-ethyl-3-methyl- 1H-pyrazol-4-yl)-6- fluoro-2-pyridyl]amino]- 2-oxo-ethyl]-3- isopropyl-isoxazole-4- carboxamide | 2.43 | 509.268 |
| 22 | 111 | 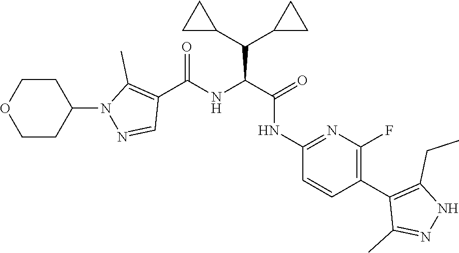 [SMILES: CCc1[nH]nc(C)c1-c1ccc(NC(=O)[C@@H](NC(=O)c2cnn(C3CCOCC3)c2C)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-5-methyl- 1-tetrahydropyran-4-yl- pyrazole-4-carboxamide | 2.25 | 564.310 |
| 23 | 97 | 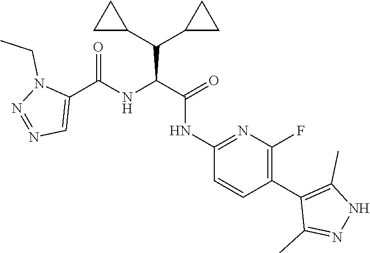 [SMILES: CCn1nncc1C(=O)N[C@H](C(=O)Nc1ccc(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-6-fluoro-2- pyridyl]amino]-2-oxo- ethyl]-3-ethyl-triazole-4- carboxamide | 2.21 | 481.236 |
| 24 | 93 | 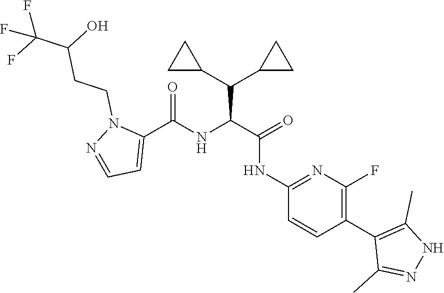 [SMILES: Cc1n[nH]c(C)c1-c1ccc(NC(=O)[C@@H](NC(=O)c2ccnn2CCC(O)C(F)(F)F)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(4,4,4-trifluoro- 3-hydroxy- butyl)pyrazole-3- carboxamide (Diastereomer 1) | 2.32 | 578.250 |
| 25 | 94 | 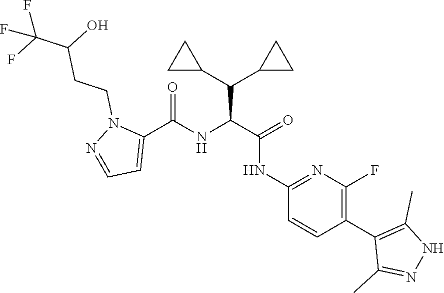 [SMILES: Cc1n[nH]c(C)c1-c1ccc(NC(=O)[C@@H](NC(=O)c2ccnn2CCC(O)C(F)(F)F)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(4,4,4-trifluoro- 3-hydroxy- butyl)pyrazole-3- carboxamide (Diastereomer 2) | 2.32 | 578.251 |
| 26 | 116 | 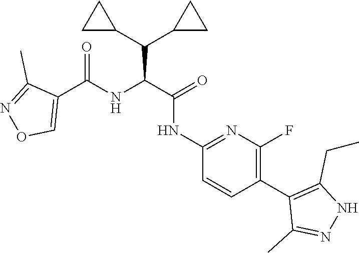 [SMILES: CCc1[nH]nc(C)c1-c1ccc(NC(=O)[C@@H](NC(=O)c2conc2C)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-3-methyl- isoxazole-4-carboxamide | 2.29 | 481.237 |
| 27 | 115 | 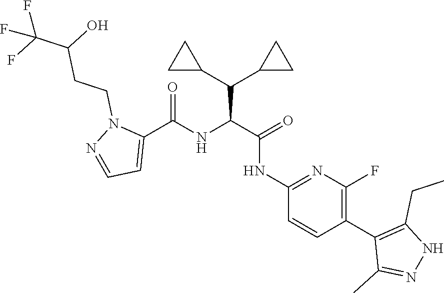 [SMILES: CCc1[nH]nc(C)c1-c1ccc(NC(=O)[C@@H](NC(=O)c2ccnn2CCC(O)C(F)(F)F)C(C2CC2)C2CC2)nc1F] | N-[(1S)-1-(dicyclo- propylmethyl)-2-[[5-(5- ethyl-3-methyl-1H- pyrazol-4-yl)-6-fluoro-2- pyridyl]amino]-2-oxo- ethyl]-2-(4,4,4-trifluoro- 3-hydroxy-butyl)- pyrazole-3-carboxamide (Diastereomer 2) | 2.36 | 592.266 |
| 28 | 114 | 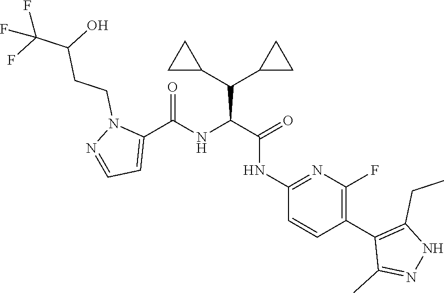 [SMILES: CCc1[nH]nc(C)c1-c1ccc(NC(=O)[C@@H](NC(=O)c2ccnn2CCC(O)C(F)(F)F)C(C2CC2)C2CC2)nc1F] | N-[(1S)-1-(dicyclo- propylmethyl)-2-[[5-(5- ethyl-3-methyl-1H- pyrazol-4-yl)-6-fluoro-2- pyridyl]amino]-2-oxo- ethyl]-2-(4,4,4-trifluoro- 3-hydroxy-butyl)- pyrazole-3-carboxamide (Diastereomer 1) | 2.36 | 592.266 |
| 29 | 52 | 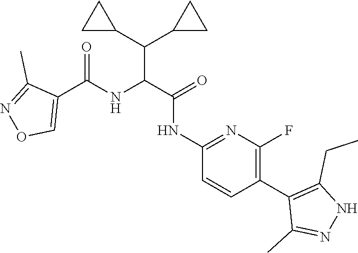 [SMILES: CCc1[nH]nc(C)c1-c1ccc(NC(=O)C(NC(=O)c2conc2C)C(C2CC2)C2CC2)nc1F] | N-[1- (dicyclopropylmethyl)-2- [[5-(5-ethyl-3-methyl- 1H-pyrazol-4-yl)-6- fluoro-2-pyridyl]amino]- 2-oxo-ethyl]-3-methyl- isoxazole-4-carboxamide | 2.29 | 451.237 |
| 30 | 104 | 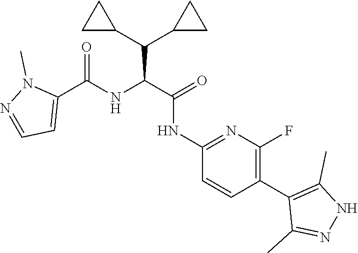 [SMILES: Cc1n[nH]c(C)c1-c1ccc(NC(=O)[C@@H](NC(=O)c2ccnn2C)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-methyl- pyrazole-3-carboxamide | 2.20 | 466.236 |
| 31 | 105 | 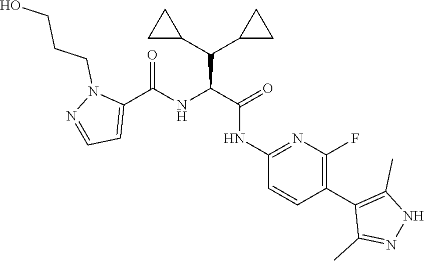 [SMILES: Cc1n[nH]c(C)c1-c1ccc(NC(=O)[C@@H](NC(=O)c2ccnn2CCCO)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(3- hydroxypropyl)pyrazole- 3-carboxamide | 2.13 | 480.252 |
| 32 | 109 | 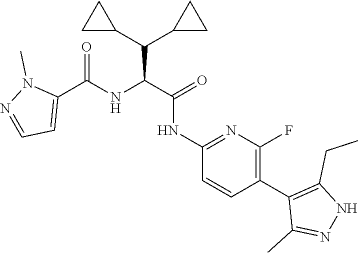 [SMILES: CCc1[nH]nc(C)c1-c1ccc(NC(=O)[C@@H](NC(=O)c2ccnn2C)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-methyl- pyrazole-3-carboxamide | 2.26 | 480.252 |
| 33 | 98 | 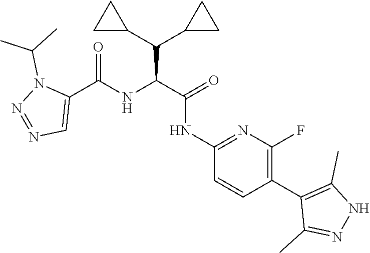 [SMILES: Cc1n[nH]c(C)c1-c1ccc(NC(=O)[C@@H](NC(=O)c2cnnn2C(C)C)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-3-isopropyl- triazole-4-carboxamide | 2.26 | 495.263 |
| 34 | 107 | 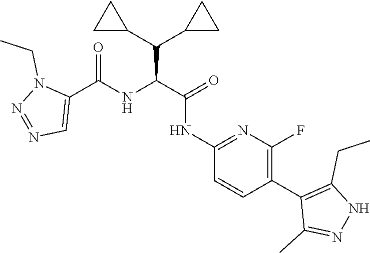 [SMILES: CCc1[nH]nc(C)c1-c1ccc(NC(=O)[C@@H](NC(=O)c2cnnn2CC)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-3-ethyl- triazole-4-carboxamide | 2.26 | 495.263 |
| 35 | 118 | 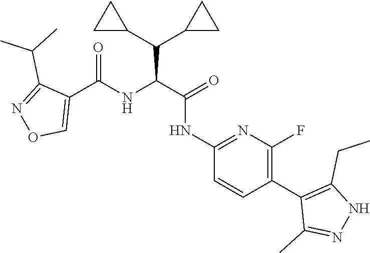 [SMILES: CCc1[nH]nc(C)c1-c1ccc(NC(=O)[C@@H](NC(=O)c2conc2C(C)C)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-3- isopropyl-isoxazole-4- carboxamide | 2.43 | 509.268 |
| 36 | 80 | 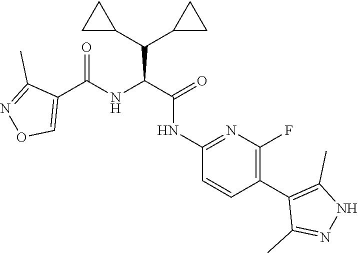 [SMILES: Cc1nocc1C(=O)N[C@H](C(=O)Nc1ccc(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-6-fluoro-2- pyridyl]amino]-2-oxo- ethyl]-3-methyl- isoxazole-4-carboxamide | 2.24 | 467.221 |
| 37 | 53 | 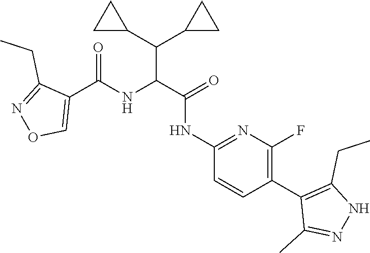 [SMILES: CCc1nocc1C(=O)NC(C(=O)Nc1ccc(-c2c(C)n[nH]c2CC)c(F)n1)C(C1CC1)C1CC1] | N-[1- (dicyclopropylmethyl)-2- [[5-(5-ethyl-3-methyl- 1H-pyrazol-4-yl)-6- fluoro-2-pyridyl]amino]- 2-oxo-ethyl]-3-ethyl- isoxazole-4-carboxamide | 2.36 | 495.252 |
| 38 | 82 | 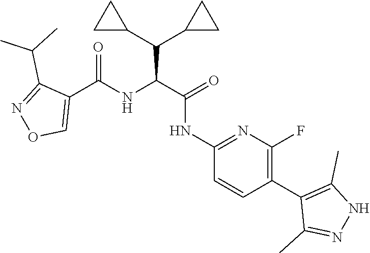 [SMILES: Cc1n[nH]c(C)c1-c1ccc(NC(=O)[C@@H](NC(=O)c2conc2C(C)C)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-3-isopropyl- isoxazole-4-carboxamide | 2.38 | 495.252 |
| 39 | 106 | 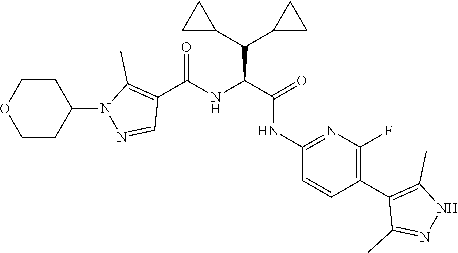 [SMILES: Cc1n[nH]c(C)c1-c1ccc(NC(=O)[C@@H](NC(=O)c2cnn(C3CCOCC3)c2C)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-5-methyl-1- tetrahydropyran-4-yl- pyrazole-4-carboxamide | 2.20 | 550.294 |
| 40 | 110 | 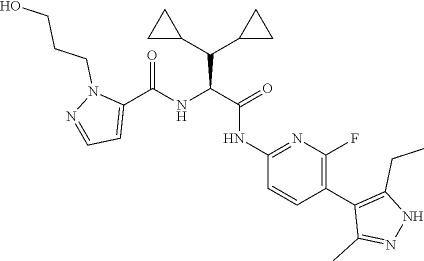 [SMILES: CCc1[nH]nc(C)c1-c1ccc(NC(=O)[C@@H](NC(=O)c2ccnn2CCCO)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-(3- hydroxypropyl)pyrazole- 3-carboxamide | 2.18 | 524.279 |
| 41 | 108 | 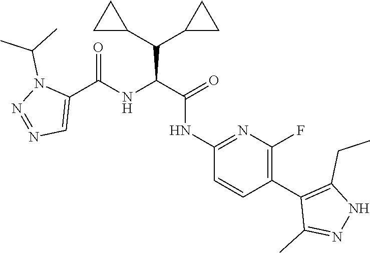 [SMILES: CCc1[nH]nc(C)c1-c1ccc(NC(=O)[C@@H](NC(=O)c2cnnn2C(C)C)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-3- isopropyl-triazole-4- carboxamide | 2.31 | 509.279 |
| 42 | 117 | 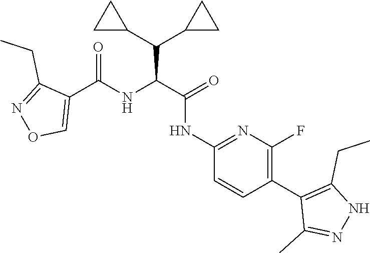 [SMILES: CCc1nocc1C(=O)N[C@H](C(=O)Nc1ccc(-c2c(C)n[nH]c2CC)c(F)n1)C(C1CC1)C1CC1] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-3-ethyl- isoxazole-4-carboxamide | 2.36 | 495.252 |
| 66 | 141 | 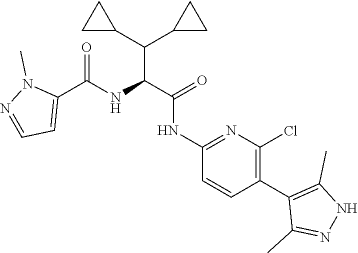 [SMILES: Cc1n[nH]c(C)c1-c1ccc(NC(=O)[C@@H](NC(=O)c2ccnn2C)C(C2CC2)C2CC2)nc1Cl] | N-[(1S)-1-[[6-chloro-5- (3,5-dimethyl-1H- pyrazol-4-yl)-2- pyridyl]carbamoyl]-2,2- dicyclopropyl-ethyl]-2- methyl-pyrazole-3- carboxamide | 2.24 | 482.207 |
| 67 | 142 | 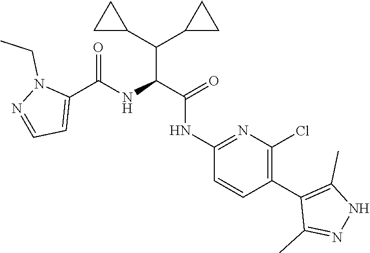 [SMILES: CCn1nccc1C(=O)N[C@H](C(=O)Nc1ccc(-c2c(C)n[nH]c2C)c(Cl)n1)C(C1CC1)C1CC1] | N-[(1S)-1-[[6-chloro-5- (3,5-dimethyl-1H- pyrazol-4-yl)-2- pyridyl]carbamoyl]-2,2- dicyclopropyl-ethyl]-2- ethyl-pyrazole-3- carboxamide | 2.30 | 496.223 |
| 68 | 143 | 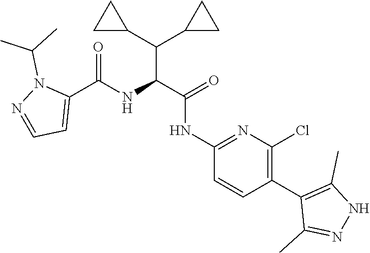 [SMILES: Cc1n[nH]c(C)c1-c1ccc(NC(=O)[C@@H](NC(=O)c2ccnn2C(C)C)C(C2CC2)C2CC2)nc1Cl] | N-[(1S)-1-[[6-chloro-5- (3,5-dimethyl-1H- pyrazol-4-yl)-2- pyridyl]carbamoyl]-2,2- dicyclopropyl-ethyl]-2- isopropyl-pyrazole-3- carboxamide | 2.38 | 510.238 |
| 69 | 144 | 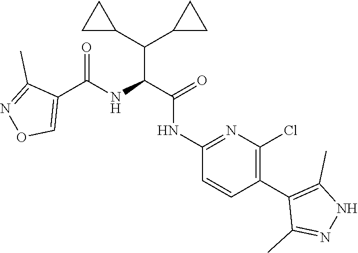 [SMILES: Cc1nocc1C(=O)N[C@H](C(=O)Nc1ccc(-c2c(C)n[nH]c2C)c(Cl)n1)C(C1CC1)C1CC1] | N-[(1S)-1-[[6-chloro-5- (3,5-dimethyl-1H- pyrazol-4-yl)-2- pyridyl]carbamoyl]-2,2- dicyclopropyl-ethyl]-3- methyl-isoxazole-4- carboxamide | 2.27 | 483.191 |
| 70 | 145 | 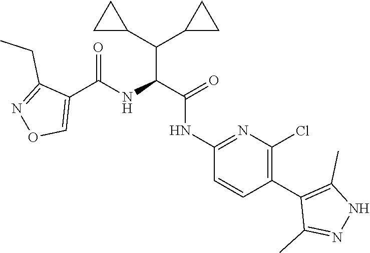 [SMILES: CCc1nocc1C(=O)N[C@H](C(=O)Nc1ccc(-c2c(C)n[nH]c2C)c(Cl)n1)C(C1CC1)C1CC1] | N-[(1S)-1-[[6-chloro-5- (3,5-dimethyl-1H- pyrazol-4-yl)-2- pyridyl]carbamoyl]-2,2- dicyclopropyl-ethyl]-3- ethyl-isoxazole-4- carboxamide | 2.34 | 497.207 |
| 71 | 146 | 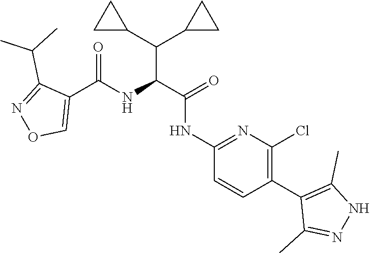 [SMILES: Cc1n[nH]c(C)c1-c1ccc(NC(=O)[C@@H](NC(=O)c2conc2C(C)C)C(C2CC2)C2CC2)nc1Cl] | N-[(1S)-1-[[6-chloro-5- (3,5-dimethyl-1H- pyrazol-4-yl)-2- pyridyl]carbamoyl]-2,2- dicyclopropyl-ethyl]-3- isopropyl-isoxazole-4- carboxamide | 2.41 | 511.223 |
| 72 | 163 | 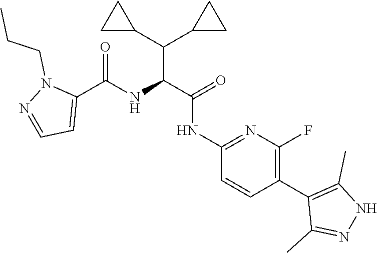 [SMILES: CCCn1nccc1C(=O)N[C@H](C(=O)Nc1ccc(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-propyl- pyrazole-3-carboxamide | 2.34 | 494.268 |
| 73 | 165 | 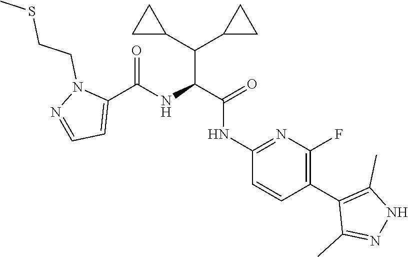 [SMILES: CSCCn1nccc1C(=O)N[C@H](C(=O)Nc1ccc(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(2- methylsulfanylethyl) pyrazole-3-carboxamide | 2.34 | 526.24 |
| 74 | 167 | 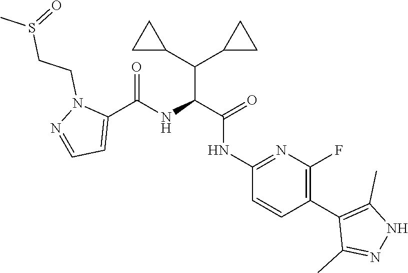 [SMILES: Cc1n[nH]c(C)c1-c1ccc(NC(=O)[C@@H](NC(=O)c2ccnn2CCS(C)=O)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(2- methylsulfinylethyl) pyrazole-3-carboxamide | 2.07 | 542.235 |
| 75 | 169 | 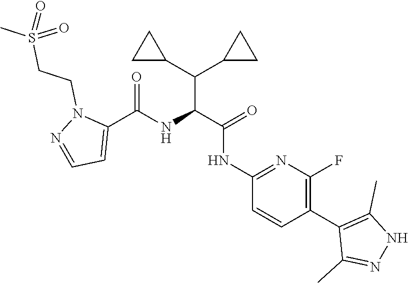 [SMILES: Cc1n[nH]c(C)c1-c1ccc(NC(=O)[C@@H](NC(=O)c2ccnn2CCS(C)(=O)=O)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(2- methylsulfonylethyl) pyrazole-3-carboxamide | 2.17 | 558.23 |
| 78 | 173 | 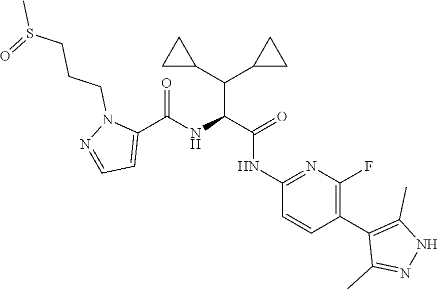 [SMILES: Cc1n[nH]c(C)c1-c1ccc(NC(=O)[C@@H](NC(=O)c2ccnn2CCCS(C)=O)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(3- methylsulfinylpropyl) pyrazole-3-carboxamide | 2.07 | 556.251 |
| 79 | 175 | 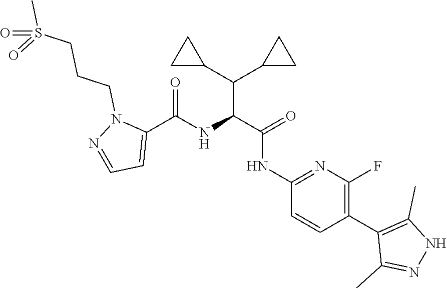 [SMILES: Cc1n[nH]c(C)c1-c1ccc(NC(=O)[C@@H](NC(=O)c2ccnn2CCCS(C)(=O)=O)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(3- methylsulfonylpropyl) pyrazole-3-carboxamide | 2.16 | 572.246 |
| 80 | 176 | 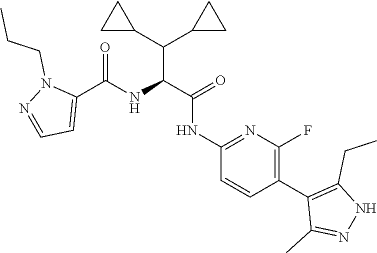 [SMILES: CCCn1nccc1C(=O)N[C@H](C(=O)Nc1ccc(-c2c(C)n[nH]c2CC)c(F)n1)C(C1CC1)C1CC1] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-propyl- pyrazole-3-carboxamide | 2.39 | 508.284 |
| 81 | 177 | 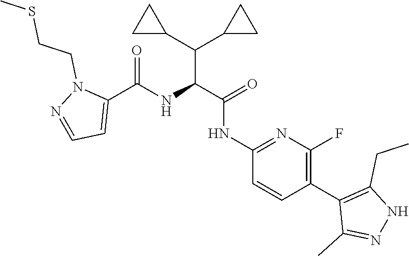 [SMILES: CCc1[nH]nc(C)c1-c1ccc(NC(=O)[C@@H](NC(=O)c2ccnn2CCSC)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-(2- methylsulfanylethyl) pyrazole-3-carboxamide | 2.39 | 540.256 |
| 82 | 178 | 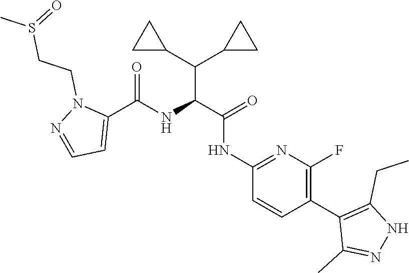 [SMILES: CCc1[nH]nc(C)c1-c1ccc(NC(=O)[C@@H](NC(=O)c2ccnn2CCS(C)=O)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-(2- methylsulfinylethyl) pyrazole-3-carboxamide | 2.12 | 556.252 |
| 83 | 179 | 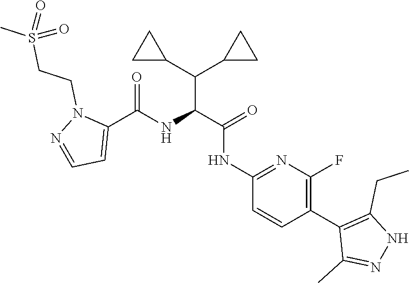 [SMILES: CCc1[nH]nc(C)c1-c1ccc(NC(=O)[C@@H](NC(=O)c2ccnn2CCS(C)(=O)=O)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-(2- methylsulfonylethyl) pyrazole-3-carboxamide | 2.22 | 572.246 |
| 84 | 180 | 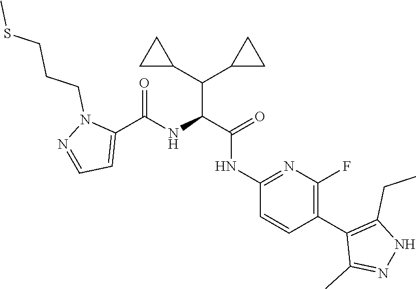 [SMILES: CCc1[nH]nc(C)c1-c1ccc(NC(=O)[C@@H](NC(=O)c2ccnn2CCCSC)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-(3- methylsulfanylpropyl) pyrazole-3-carboxamide | 2.43 | 554.271 |
| 85 | 181 | 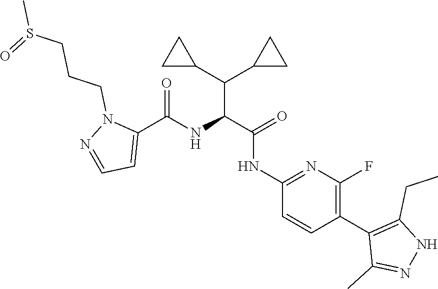 [SMILES: CCc1[nH]nc(C)c1-c1ccc(NC(=O)[C@@H](NC(=O)c2ccnn2CCCS(C)=O)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-(3- methylsulfinylpropyl) pyrazole-3-carboxamide | 2.11 | 570.266 |
| 86 | 182 | 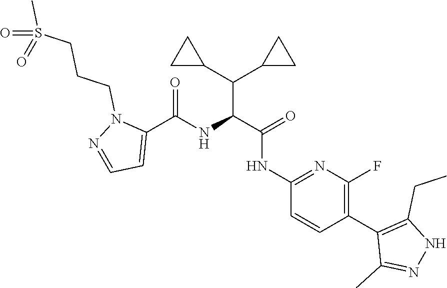 [SMILES: CCc1[nH]nc(C)c1-c1ccc(NC(=O)[C@@H](NC(=O)c2ccnn2CCCS(C)(=O)=O)C(C2CC2)C2CC2)nc1F] | N-[(1S)-1- (dicyclopropylmethyl)-2- [[5-(5-ethyl-3-methyl- 1H-pyrazol-4-yl)-6- fluoro-2-pyridyl]amino]- 2-oxo-ethyl]-2-(3- methylsulfonylpropyl) pyrazole-3-carboxamide | 2.21 | 586.261 |
| 90 | 195 | 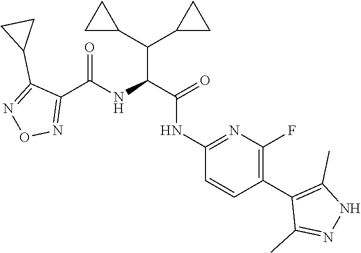 [SMILES: Cc1n[nH]c(C)c1-c1ccc(NC(=O)[C@@H](NC(=O)c2nonc2C2CC2)C(C2CC2)C2CC2)nc1F] | 4-cyclopropyl-N-[(1S)-1- (dicyclopropylmethyl)-2- [[5-(3,5-dimethyl-1H- pyrazol-4-yl)-6-fluoro-2- pyridyl]amino]-2-oxo- ethyl]-1,2,5-oxadiazole- 3-carboxamide | 2.50 | 494.232 |
| 91 | 199 | 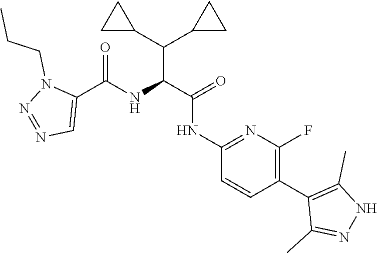 [SMILES: CCCn1nncc1C(=O)N[C@H](C(=O)Nc1ccc(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-6-fluoro-2- pyridyl]amino]-2-oxo- ethyl]-3-propyl-triazole- 4-carboxamide | 2.27 | 495.263 |
| 94 | 206 | 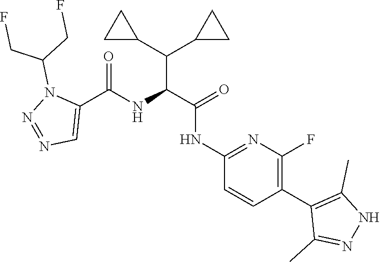 [SMILES: Cc1n[nH]c(C)c1-c1ccc(NC(=O)[C@@H](NC(=O)c2cnnn2C(CF)CF)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-3-[2-fluoro-1- (fluoromethyl)ethyl] triazole-4-carboxamide | 2.26 | 531.245 |
| 95 | 209 | 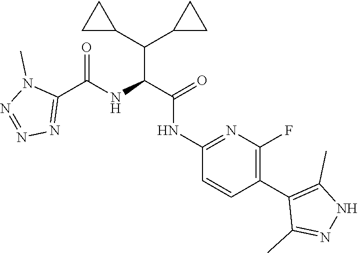 [SMILES: Cc1n[nH]c(C)c1-c1ccc(NC(=O)[C@@H](NC(=O)c2nnnn2C)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-1-methyl- tetrazole-5-carboxamide | 2.25 | 468.227 |
| 100 | 212 | 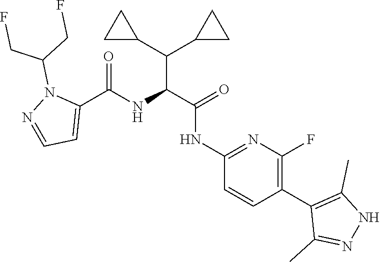 [SMILES: Cc1n[nH]c(C)c1-c1ccc(NC(=O)[C@@H](NC(=O)c2ccnn2C(CF)CF)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-[2-fluoro-1- (fluoromethyl)ethyl] pyrazole-3-carboxamide | 2.33 | 530.249 |
| 104 | 223 | 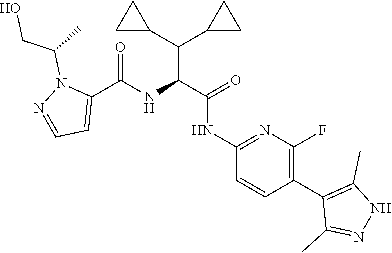 [SMILES: Cc1n[nH]c(C)c1-c1ccc(NC(=O)[C@@H](NC(=O)c2ccnn2[C@@H](C)CO)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-[(1S)-2- hydroxy-1-methyl- ethyl]pyrazole-3- carboxamide | 2.13 | 510.263 |
| 120 | 245 | 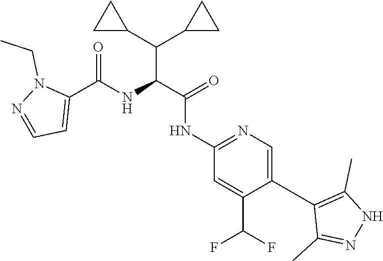 [SMILES: CCn1nccc1C(=O)N[C@H](C(=O)Nc1cc(C(F)F)c(-c2c(C)n[nH]c2C)cn1)C(C1CC1)C1CC1] | N-[(1S)-1- (dicyclopropylmethyl)-2- [[4-(difluoromethyl)-5- (3,5-dimethyl-1H- pyrazol-4-yl)-2- pyridyl]amino]-2-oxo- ethyl]-2-ethyl-pyrazole- 3-carboxamide | 2.29 | 512.259 |
| 121 | 246 | 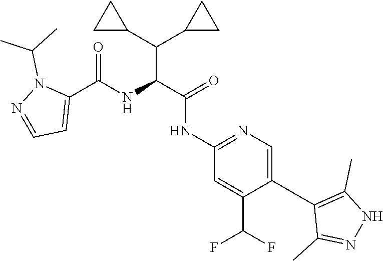 [SMILES: Cc1n[nH]c(C)c1-c1cnc(NC(=O)[C@@H](NC(=O)c2ccnn2C(C)C)C(C2CC2)C2CC2)cc1C(F)F] | N-[(1S)-1- (dicyclopropylmethyl)-2- [[4-(difluoromethyl)-5- (3,5-dimethyl-1H- pyrazol-4-yl)-2- pyridyl]amino]-2-oxo- ethyl]-2-isopropyl- pyrazole-3-carboxamide | 2.36 | 526.274 |
| 125 | 264 | 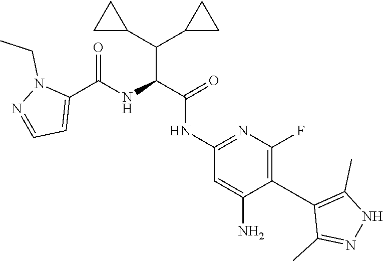 [SMILES: CCn1nccc1C(=O)N[C@H](C(=O)Nc1cc(N)c(-c2c(C)n[nH]c2C)c(F)n1)C(C1CC1)C1CC1] | N-[(1S)-1-[[4-amino-5- (3,5-dimethyl-1H- pyrazol-4-yl)-6-fluoro-2- pyridyl]carbamoyl]-2,2- dicyclopropyl-ethyl]-2- ethyl-pyrazole-3- carboxamide | 2.17 | 495.263 |
| 126 | 269 | 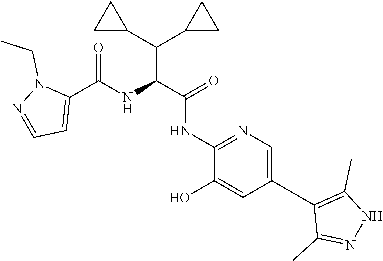 [SMILES: CCn1nccc1C(=O)N[C@H](C(=O)Nc1ncc(-c2c(C)n[nH]c2C)cc1O)C(C1CC1)C1CC1] | N-[(1S)-1- (dicyclopropylmethyl)-2- [[5-(3,5-dimethyl-1H- pyrazol-4-yl)-3-hydroxy- 2-pyridyl]amino]-2-oxo- ethyl]-2-ethyl-pyrazole- 3-carboxamide | 2.16 | 478.257 |
| 127 | 270 | 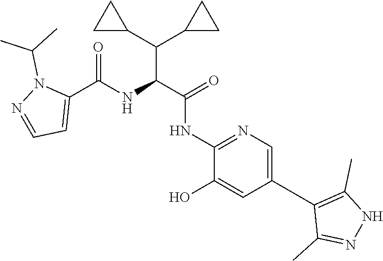 [SMILES: Cc1n[nH]c(C)c1-c1cnc(NC(=O)[C@@H](NC(=O)c2ccnn2C(C)C)C(C2CC2)C2CC2)c(O)c1] | N-[(1S)-1- (dicyclopropylmethyl)-2- [[5-(3,5-dimethyl-1H- pyrazol-4-yl)-3-hydroxy- 2-pyridyl]amino]-2-oxo- ethyl]-2-isopropyl- pyrazole-3-carboxamide | 2.24 | 492.273 |
| 128 | 273 | 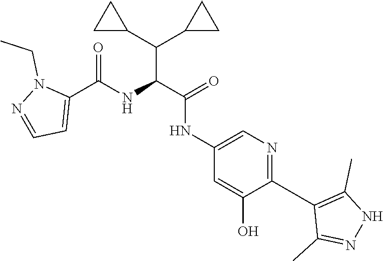 [SMILES: CCn1nccc1C(=O)N[C@H](C(=O)Nc1cnc(-c2c(C)n[nH]c2C)c(O)c1)C(C1CC1)C1CC1] | N-[(1S)-1- (dicyclopropylmethyl)-2- [[6-(3,5-dimethyl-1H- pyrazol-4-yl)-5-hydroxy- 3-pyridyl]amino]-2-oxo- ethyl]-2-ethyl-pyrazole- 3-carboxamide | 1.94 | 478.257 |
| 129 | 274 | 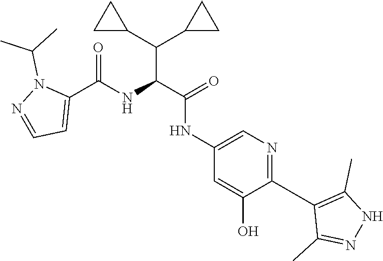 [SMILES: Cc1n[nH]c(C)c1-c1ncc(NC(=O)[C@@H](NC(=O)c2ccnn2C(C)C)C(C2CC2)C2CC2)cc1O] | N-[(1S)-1- (dicyclopropylmethyl)-2- [[6-(3,5-dimethyl-1H- pyrazol-4-yl)-5-hydroxy- 3-pyridyl]amino]-2-oxo- ethyl]-2-isopropyl- pyrazole-3-carboxamide | 2.00 | 492.273 |
| 130 | 275 | 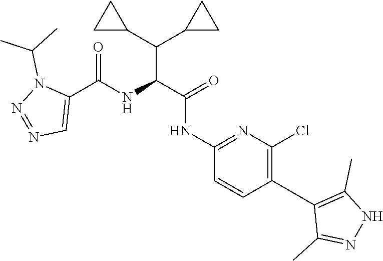 [SMILES: Cc1n[nH]c(C)c1-c1ccc(NC(=O)[C@@H](NC(=O)c2cnnn2C(C)C)C(C2CC2)C2CC2)nc1Cl] | N-[(1S)-1-[[6-chloro-5- (3,5-dimethyl-1H- pyrazol-4-yl)-2- pyridyl]carbamoyl]-2,2- dicyclopropyl-ethyl]-3- isopropyl-triazole-4- carboxamide | 2.30 | 511.234 |
| 137 | 287 | 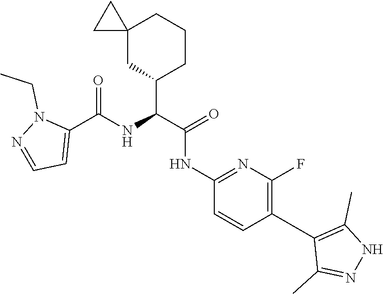 [SMILES: CCn1nccc1C(=O)N[C@H](C(=O)Nc1ccc(-c2c(C)n[nH]c2C)c(F)n1)[C@H]1CCCC2(CC2)C1] | N-[(1S)-2-[[5-(3,5- dimethyl-1H-pyrazol-4- yl)-6-fluoro-2- pyridyl]amino]-2-oxo-1- [(7S)-spiro[2.5]octan-7- yl]ethyl]-2-ethyl- pyrazole-3-carboxamide | 2.39 | 494.268 |
| 138 | 288 | 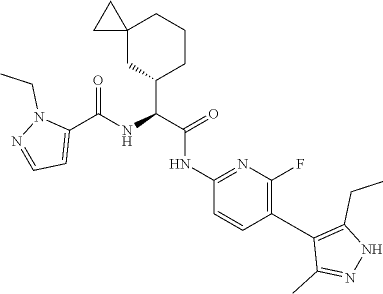 [SMILES: CCc1[nH]nc(C)c1-c1ccc(NC(=O)[C@@H](NC(=O)c2ccnn2CC)[C@H]2CCCC3(CC3)C2)nc1F] | 2-ethyl-N-[(1S)-2-[[5- (5-ethyl-3-methyl-1H- pyrazol-4-yl)-6-fluoro-2- pyridyl]amino]-2-oxo-1- [(7S)-spiro[2.5]octan-7- yl]ethyl]pyrazole-3- carboxamide | 2.44 | 508.283 |
| 139 | 297 | 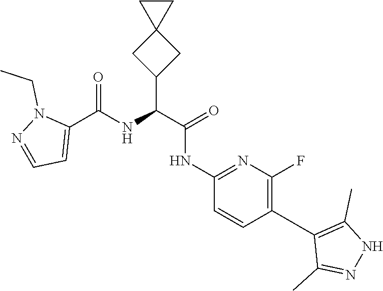 [SMILES: CCn1nccc1C(=O)N[C@H](C(=O)Nc1ccc(-c2c(C)n[nH]c2C)c(F)n1)C1CC2(CC2)C1] | N-[(1S)-2-[[5-(3,5- dimethyl-1H-pyrazol-4- yl)-6-fluoro-2- pyridyl]amino]-2-oxo-1- spiro[2.3]hexan-5-yl- ethyl]-2-ethyl-pyrazole- 3-carboxamide | 2.27 | 466.237 |
| 140 | 300 | 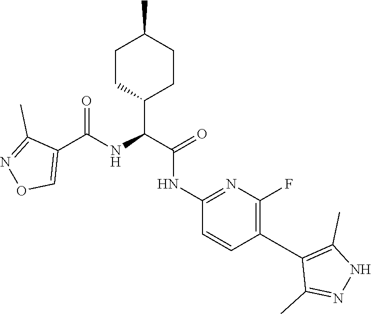 [SMILES: Cc1nocc1C(=O)N[C@H](C(=O)Nc1ccc(-c2c(C)n[nH]c2C)c(F)n1)[C@H]1CC[C@H](C)CC1] | N-[(1S)-2-[[5-(3,5- dimethyl-1H-pyrazol-4- yl)-6-fluoro-2- pyridyl]amino]-1-(4- methylcyclohexyl)-2- oxo-ethyl]-3-methyl- isoxazole-4-carboxamide | 2.34 | 469.236 |
| 141 | 301 | 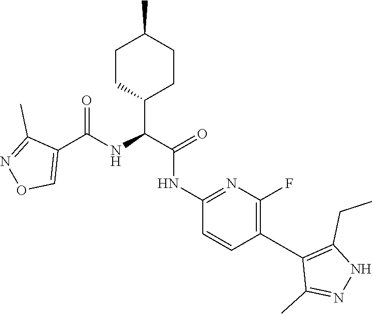 [SMILES: CCc1[nH]nc(C)c1-c1ccc(NC(=O)[C@@H](NC(=O)c2conc2C)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-3-methyl- isoxazole-4-carboxamide | 2.40 | 483.252 |
| 142 | 303 | 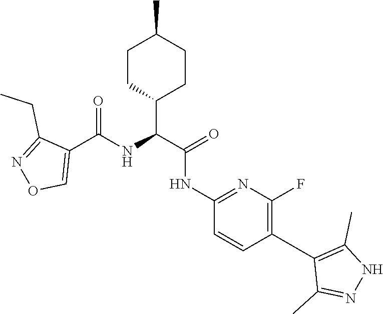 [SMILES: CCc1nocc1C(=O)N[C@H](C(=O)Nc1ccc(-c2c(C)n[nH]c2C)c(F)n1)[C@H]1CC[C@H](C)CC1] | N-[(1S)-2-[[5-(3,5- dimethyl-1H-pyrazol-4- yl)-6-fluoro-2- pyridyl]amino]-1-(4- methylcyclohexyl)-2- oxo-ethyl]-3-ethyl- isoxazole-4-carboxamide | 2.42 | 483.252 |
| 143 | 304 | 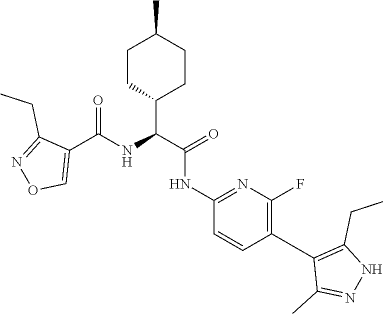 [SMILES: CCc1nocc1C(=O)N[C@H](C(=O)Nc1ccc(-c2c(C)n[nH]c2CC)c(F)n1)[C@H]1CC[C@H](C)CC1] | 3-ethyl-N-[(1S)-2-[[5- (5-ethyl-3-methyl-1H- pyrazol-4-yl)-6-fluoro-2- pyridyl]amino]-1-(4- methylcyclohexyl)-2- oxo-ethyl]isoxazole-4- carboxamide | 2.47 | 497.268 |
| 144 | 306 | 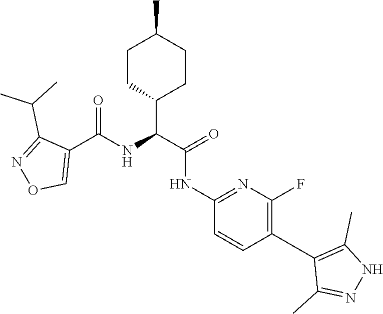 [SMILES: Cc1n[nH]c(C)c1-c1ccc(NC(=O)[C@@H](NC(=O)c2conc2C(C)C)[C@H]2CC[C@H](C)CC2)nc1F] | N-[(1S)-2-[[5-(3,5- dimethyl-1H-pyrazol-4- yl)-6-fluoro-2- pyridyl]amino]-1-(4- methylcyclohexyl)-2- oxo-ethyl]-3-isopropyl- isoxazole-4-carboxamide | 2.48 | 497.268 |
| 145 | 307 | 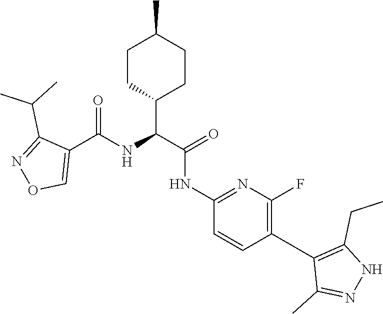 [SMILES: CCc1[nH]nc(C)c1-c1ccc(NC(=O)[C@@H](NC(=O)c2conc2C(C)C)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-3-isopropyl- isoxazole-4-carboxamide | 2.54 | 511.283 |
| 146 | 309 | 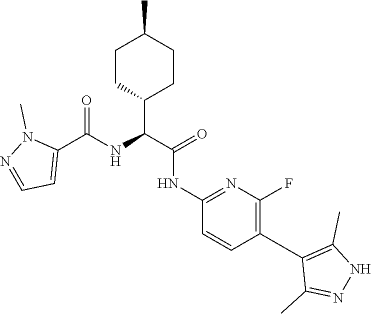 [SMILES: Cc1n[nH]c(C)c1-c1ccc(NC(=O)[C@@H](NC(=O)c2ccnn2C)[C@H]2CC[C@H](C)CC2)nc1F] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-methyl- pyrazole-3-carboxamide | 2.31 | 468.252 |
| 147 | 310 | 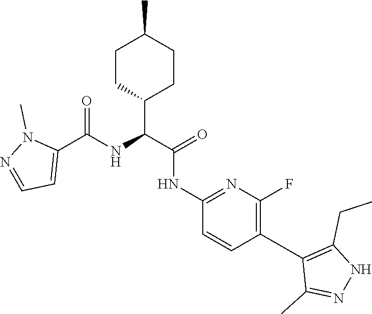 [SMILES: CCc1[nH]nc(C)c1-c1ccc(NC(=O)[C@@H](NC(=O)c2ccnn2C)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-methyl- pyrazole-3-carboxamide | 2.37 | 482.268 |
| 148 | 312 | 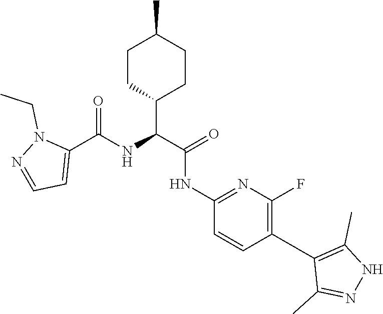 [SMILES: CCn1nccc1C(=O)N[C@H](C(=O)Nc1ccc(-c2c(C)n[nH]c2C)c(F)n1)[C@H]1CC[C@H](C)CC1] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-ethyl- pyrazole-3-carboxamide | 2.38 | 482.268 |
| 149 | 313 | 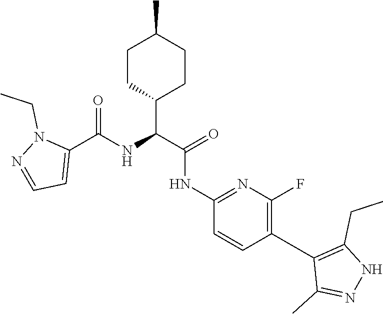 [SMILES: CCc1[nH]nc(C)c1-c1ccc(NC(=O)[C@@H](NC(=O)c2ccnn2CC)[C@H]2CC[C@H](C)CC2)nc1F] | 2-ethyl-N-[(1S)-2-[[5- (5-ethyl-3-methyl-1H- pyrazol-4-yl)-6-fluoro-2- pyridyl]amino]-1-(4- methylcyclohexyl)-2- oxo-ethyl]pyrazole-3- carboxamide | 2.43 | 496.284 |
| 150 | 315 | 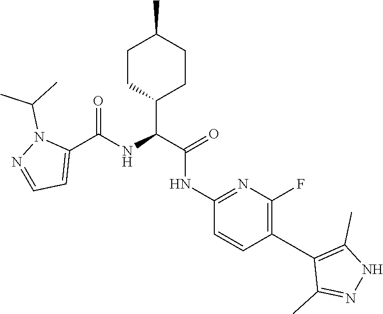 [SMILES: Cc1n[nH]c(C)c1-c1ccc(NC(=O)[C@@H](NC(=O)c2ccnn2C(C)C)[C@H]2CC[C@H](C)CC2)nc1F] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-isopropyl- pyrazole-3-carboxamide | 2.45 | 496.284 |
| 151 | 316 | 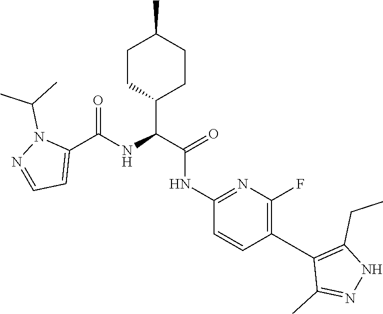 [SMILES: CCc1[nH]nc(C)c1-c1ccc(NC(=O)[C@@H](NC(=O)c2ccnn2C(C)C)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-isopropyl- pyrazole-3-carboxamide | 2.51 | 510.299 |
| 152 | 318 | 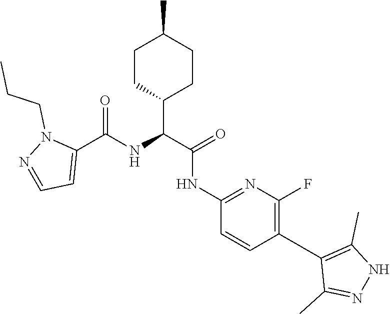 [SMILES: CCCn1nccc1C(=O)N[C@H](C(=O)Nc1ccc(-c2c(C)n[nH]c2C)c(F)n1)[C@H]1CC[C@H](C)CC1] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-propyl- pyrazole-3-carboxamide | 2.45 | 496.284 |
| 153 | 319 | 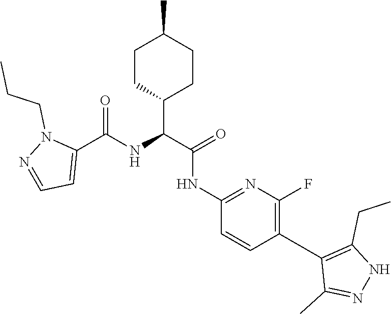 [SMILES: CCCn1nccc1C(=O)N[C@H](C(=O)Nc1ccc(-c2c(C)n[nH]c2CC)c(F)n1)[C@H]1CC[C@H](C)CC1] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-propyl- pyrazole-3-carboxamide | 2.50 | 510.299 |
| 154 | 321 | 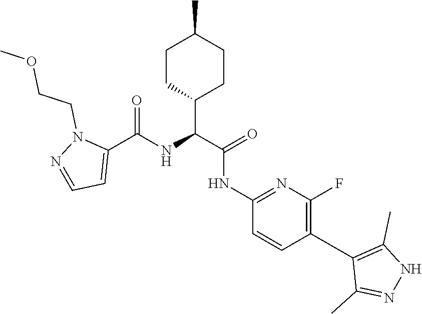 [SMILES: COCCn1nccc1C(=O)N[C@H](C(=O)Nc1ccc(-c2c(C)n[nH]c2C)c(F)n1)[C@H]1CC[C@H](C)CC1] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-(2- methoxyethyl)pyrazole- 3-carboxamide | 2.33 | 512.279 |
| 155 | 322 | 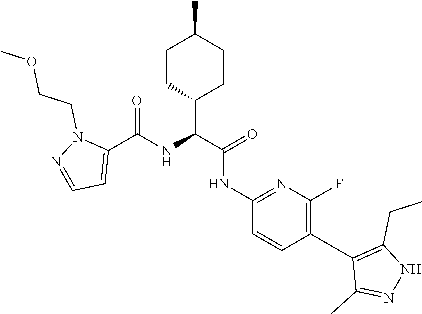 [SMILES: CCc1[nH]nc(C)c1-c1ccc(NC(=O)[C@@H](NC(=O)c2ccnn2CCOC)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-(2- methoxyethyl)pyrazole- 3-carboxamide | 2.38 | 526.294 |
| 156 | 324 | 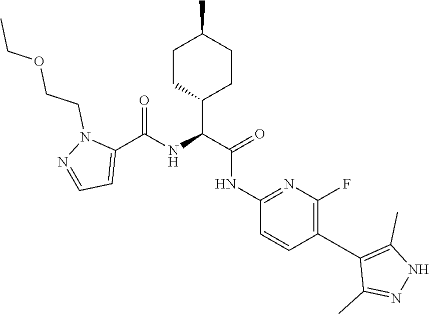 [SMILES: CCOCCn1nccc1C(=O)N[C@H](C(=O)Nc1ccc(-c2c(C)n[nH]c2C)c(F)n1)[C@H]1CC[C@H](C)CC1] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-(3- methoxypropyl)pyrazole- 3-carboxamide | 2.37 | 526.294 |
| 157 | 325 | 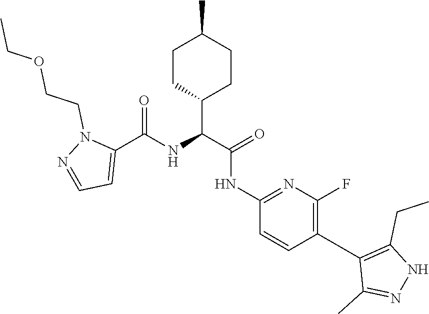 [SMILES: CCOCCn1nccc1C(=O)N[C@H](C(=O)Nc1ccc(-c2c(C)n[nH]c2CC)c(F)n1)[C@H]1CC[C@H](C)CC1] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-(3- methoxypropyl)pyrazole- 3-carboxamide | 2.42 | 540.310 |
| 158 | 329 | 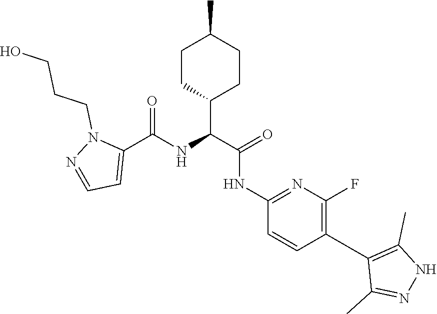 [SMILES: Cc1n[nH]c(C)c1-c1ccc(NC(=O)[C@@H](NC(=O)c2ccnn2CCCO)[C@H]2CC[C@H](C)CC2)nc1F] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-(3- hydroxypropyl)pyrazole- 3-carboxamide | 2.23 | 512.279 |
| 159 | 331 | 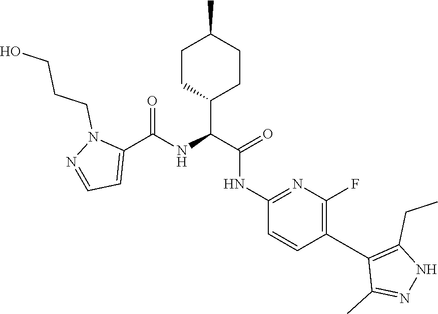 [SMILES: CCc1[nH]nc(C)c1-c1ccc(NC(=O)[C@@H](NC(=O)c2ccnn2CCCO)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-(3- hydroxypropyl)pyrazole- 3-carboxamide | 2.28 | 526.294 |
| 160 | 333 | 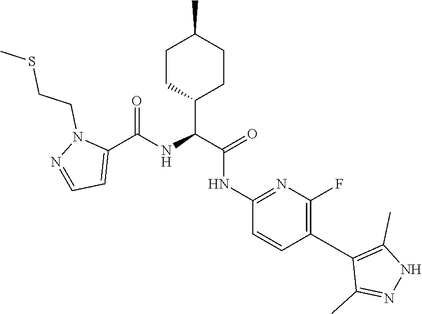 [SMILES: CSCCn1nccc1C(=O)N[C@H](C(=O)Nc1ccc(-c2c(C)n[nH]c2C)c(F)n1)[C@H]1CC[C@H](C)CC1] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-(2- methylsulfanylethyl) pyrazole-3-carboxamide | 2.44 | 528.256 |
| 161 | 334 | 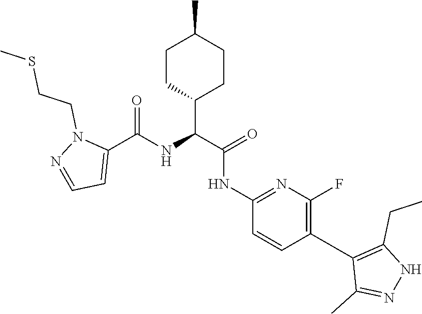 [SMILES: CCc1[nH]nc(C)c1-c1ccc(NC(=O)[C@@H](NC(=O)c2ccnn2CCSC)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-(2- methylsulfanylethyl) pyrazole-3-carboxamide | 2.49 | 542.272 |
| 166 | 339 | 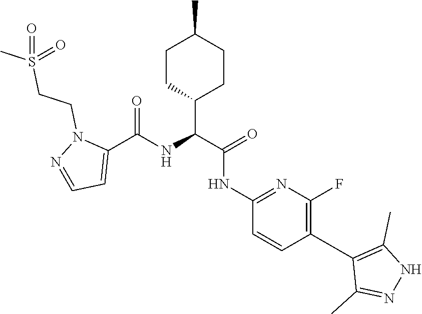 [SMILES: Cc1n[nH]c(C)c1-c1ccc(NC(=O)[C@@H](NC(=O)c2ccnn2CCS(C)(=O)=O)[C@H]2CC[C@H](C)CC2)nc1F] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-(2- methylsulfonylethyl) pyrazole-3-carboxamide | 2.26 | 560.246 |
| 167 | 340 | 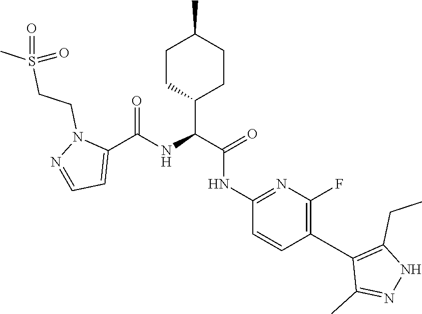 [SMILES: CCc1[nH]nc(C)c1-c1ccc(NC(=O)[C@@H](NC(=O)c2ccnn2CCS(C)(=O)=O)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-(2- methylsulfonylethyl) pyrazole-3-carboxamide | 2.32 | 574.261 |
| 176 | 363 | 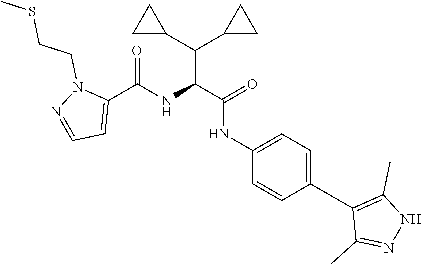 [SMILES: CSCCn1nccc1C(=O)N[C@H](C(=O)Nc1ccc(-c2c(C)n[nH]c2C)cc1)C(C1CC1)C1CC1] | N-[(1S)-1- (dicyclopropylmethyl)-2- [4-(3,5-dimethyl-1H- pyrazol-4-yl)anilino]-2- oxo-ethyl]-2-(2- methylsulfanylethyl) pyrazole-3-carboxamide | 2.29 | 507.254 |
| 179 | 365 | 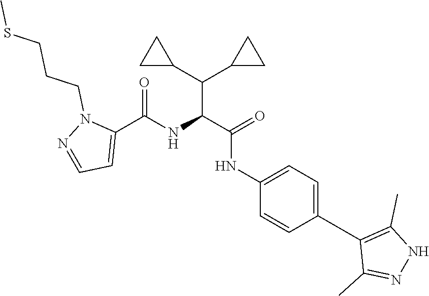 [SMILES: CSCCCn1nccc1C(=O)N[C@H](C(=O)Nc1ccc(-c2c(C)n[nH]c2C)cc1)C(C1CC1)C1CC1] | N-[(1S)-1- (dicyclopropylmethyl)-2- [4-(3,5-dimethyl-1H- pyrazol-4-yl)anilino ]-2- oxo-ethyl]-2-(3- methylsulfanylpropyl) pyrazole-3-carboxamide | 2.33 | 521.270 |
| 182 | 369 | 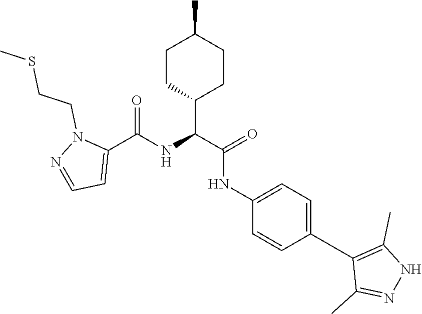 [SMILES: CSCCn1nccc1C(=O)N[C@H](C(=O)Nc1ccc(-c2c(C)n[nH]c2C)cc1)[C@H]1CC[C@H](C)CC1] | N-[(1S)-2-[4-(3,5- dimethyl-1H-pyrazol-4- yl)anilino]-1-(4- methylcyclohexyl)-2- oxo-ethyl]-2-(2- methylsulfanylethyl) pyrazole-3-carboxamide | 2.39 | 509.270 |
| 185 | 371 | 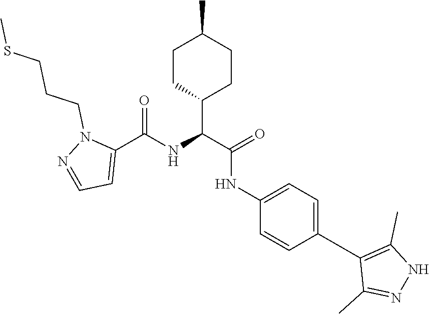 [SMILES: CSCCCn1nccc1C(=O)N[C@H](C(=O)Nc1ccc(-c2c(C)n[nH]c2C)cc1)[C@H]1CC[C@H](C)CC1] | N-[(1S)-2-[4-(3,5- dimethyl-1H-pyrazol-4- yl)anilino]-1-(4- methylcyclohexyl)-2- ox-ethyl]-2-(3- methylsulfanylpropyl) pyrazole-3-carboxamide | 2.43 | 523.285 |
| 188 | 382 | 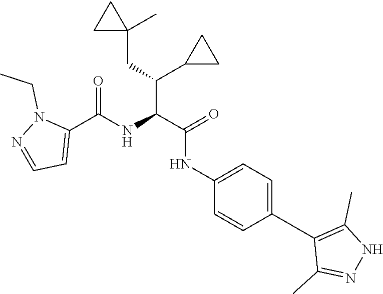 [SMILES: CCn1nccc1C(=O)N[C@H](C(=O)Nc1ccc(-c2c(C)n[nH]c2C)cc1)[C@H](CC1(C)CC1)C1CC1] | N-[(15,2R)-2- cyclopropyl-1-[[4-(3,5- dimethyl-1H-pyrazol-4- yl)phenyl] carbamoyl]-3-(1- methylcyclopropyl) propyl]-2-ethyl- pyrazole-3- carboxamide | 2.38 | 489.298 |
| 189 | 390 | 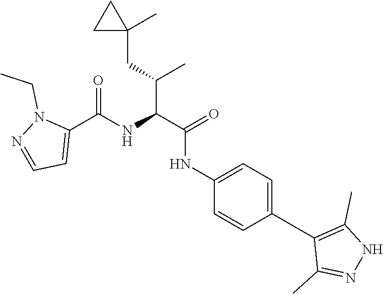 [SMILES: CCn1nccc1C(=O)N[C@H](C(=O)Nc1ccc(-c2c(C)n[nH]c2C)cc1)[C@@H](C)CC1(C)CC1] | N-[(1S,2S)-1-[[4-(3,5- dimethyl-1H-pyrazol-4- yl)phenyl]carbamoyl]-2- methyl-3-(1- methylcyclopropyl) propyl]-2-ethyl- pyrazole-3-carboxamide | 2.31 | 463.282 |
| 190 | 137 | 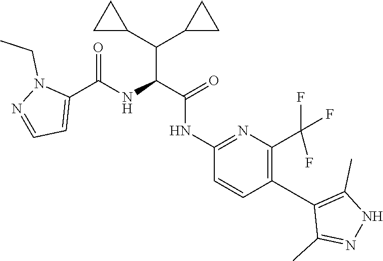 [SMILES: CCn1nccc1C(=O)N[C@H](C(=O)Nc1ccc(-c2c(C)n[nH]c2C)c(C(F)(F)F)n1)C(C1CC1)C1CC1] | N-[(1S)-1- (dicyclopropylmethyl)-2- [[5-(3,5-dimethyl-1H- pyrazol-4-yl)-6- (trifluoromethyl)-2- pyridyl]amino]-2-oxo- ethyl]-2-ethyl-pyrazole- 3-carboxamide | 2.36 | 530.249 |

### Example 103: 2-(azetidin-3-ylmethyl)-N-[(1S)-1-(dicyclopropylmethyl)-2-[[5-(3,5-dimethyl-1H-pyrazol-4-yl)-6-fluoro-2-pyridyl]amino]-2-oxo-ethyl]pyrazole-3-carboxamide

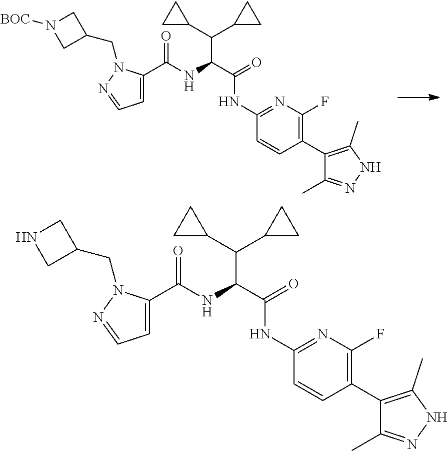

A 4M solution of hydrogen chloride in dioxane (1 mL) was added to a solution of the compound of Preparation 219 (31 mg, 0.05 mmol) in MeOH (1 mL) and the mixture was stirred for 2 hours. The solvent was removed in vacuo and the residue was purified by acidic prep. HPLC to give the title compound (3.9 mg, 15% yield). LCMS (ES): m/z 521.279 [M+H]⁺; RT=1.94 min.

### Example 43: N-[(1S)-1-(dicyclopropylmethyl)-2-[[5-(3,5-dimethyl-1H-pyrazol-4-yl)-6-methyl-2-pyridyl]amino]-2-oxo-ethyl]-2-ethyl-pyrazole-3-carboxamide and Example 44: N-[(1R)-1-(dicyclopropylmethyl)-2-[[5-(3,5-dimethyl-1H-pyrazol-4-yl)-6-methyl-2-pyridyl]amino]-2-oxo-ethyl]-2-ethyl-pyrazole-3-carboxamide

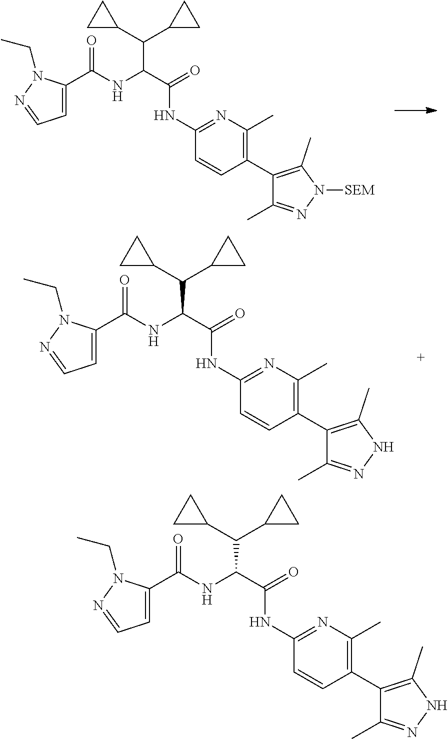

TFA (2 mL) was added to a solution of the compound of Preparation 32 (170 mg, 0.28 mmol) in DCM (2 mL) and stirred at room temperature for 2 hours. The reaction mixture was concentrated in vacuo and purified directly by prep. acidic HPLC to afford the title compounds as an off-white solid (56 mg, 42% yield). N-[1-(dicyclopropylmethyl)-2-[[5-(3,5-dimethyl-1H-pyrazol-4-yl)-6-methyl-2-pyridyl]amino]-2-oxo-ethyl]-2-ethyl-pyrazole-3-carboxamide (45 mg, 0.095 mmol) was dissolved in MeOH (1.5 mL) and separated by SFC (IC column, 40% MeOH, isocratic run) to afford the title compounds as colourless solids.

Example 43: Peak 1 (retention time 1.97 min, 6.6 mg, 15% yield); 1H NMR (600 MHz, DMSO-d6) δ 10.63 (s, 1H), 8.41 (d, J=8.7 Hz, 1H), 7.93 (d, J=8.3 Hz, 1H), 7.62-7.40 (m, 2H), 6.98 (d, J=2.0 Hz, 1H), 4.92 (t, J=8.0 Hz, 1H), 4.57-4.33 (m, 2H), 2.21 (s, 3H), 2.00 (s, 6H), 1.28 (t, J=7.1 Hz, 3H), 0.96 (ddt, J=13.4, 8.4, 4.2 Hz, 1H), 0.90-0.82 (m, 1H), 0.77 (td, J=9.4, 7.4 Hz, 1H), 0.48 (ddd, J=12.0, 8.2, 5.9 Hz, 1H), 0.38 (ddt, J=9.6, 8.3, 4.0 Hz, 1H), 0.33-0.26 (m, 2H), 0.22 (qt, J=7.8, 4.8 Hz, 4H); LCMS (ES): m/z 476.276 [M+H]⁺; RT=2.20 min.

Example 44: Peak 2 (retention time 3.59 min, 7.0 mg, 16% yield); 1H NMR (600 MHz, DMSO-d6) δ 12.30 (s, 1H), 10.62 (s, 1H), 8.41 (d, J=8.7 Hz, 1H), 7.93 (d, J=8.4 Hz, 1H), 7.60-7.34 (m, 2H), 6.98 (d, J=2.0 Hz, 1H), 4.92 (t, J=8.1 Hz, 1H), 4.65-4.29 (m, 2H), 2.21 (s, 3H), 1.99 (s, 6H), 1.29 (t, J=7.2 Hz, 3H), 0.96 (tq, J=8.4, 5.2, 4.3 Hz, 1H), 0.91-0.82 (m, 1H), 0.77 (td, J=9.4, 7.3 Hz, 1H), 0.48 (ddd, J=11.9, 8.3, 6.0 Hz, 1H), 0.38 (dq, J=12.5, 3.7 Hz, 1H), 0.34-0.25 (m, 2H), 0.26-0.17 (m, 4H); LCMS (ES): m/z 476.276 [M+H]⁺; RT=2.20 min.

The examples listed in the table below were all accessed using the method described for Example 43 and Example 44.

| | Precursor | | | | |
|---|---|---|---|---|---|
| Ex. | Prep. | | | LCMS | Mass |
| no. | number | Structure | Name | RT | ion |
| 45 | 46 | 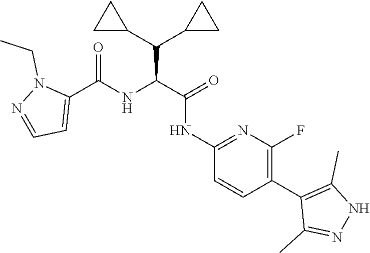 [SMILES: CCn1nccc1C(=O)N[C@H](C(=O)Nc1ccc(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-ethyl-pyrazole- 3-carboxamide | 2.25 | 480.252 |
| 46 | 46 | 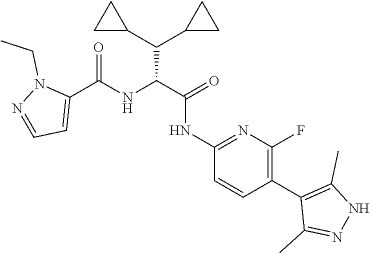 [SMILES: CCn1nccc1C(=O)N[C@@H](C(=O)Nc1ccc(-c2c(C)n[nH]c2C)c(F)n1)C(C1CC1)C1CC1] | N-[(1R)-1- (dicyclopropylmethyl)-2- [[5-(3,5-dimethyl-1H- pyrazol-4-yl)-6-fluoro-2- pyridyl]amino]-2-oxo- ethyl]-2-ethyl-pyrazole- 3-carboxamide | 2.25 | 480.252 |
| 47 | 26 | 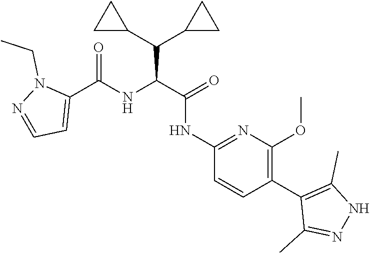 [SMILES: CCn1nccc1C(=O)N[C@H](C(=O)Nc1ccc(-c2c(C)n[nH]c2C)c(OC)n1)C(C1CC1)C1CC1] | N-[(1S)-1- (dicyclopropylmethyl)-2- [[5-(3,5-dimethyl-1H- pyrazol-4-yl)-6- methoxy-2- pyridyl]amino]-2-oxo- ethyl]-2-ethyl-pyrazole- 3-carboxamide | 2.26 | 492.272 |
| 48 | 26 | 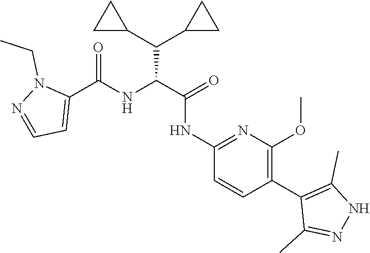 [SMILES: CCn1nccc1C(=O)N[C@@H](C(=O)Nc1ccc(-c2c(C)n[nH]c2C)c(OC)n1)C(C1CC1)C1CC1] | N-[(1R)-1- (dicyclopropylmethyl)-2- [[5-(3,5-dimethyl-1H- pyrazol-4-yl)-6- methoxy-2- pyridyl]amino]-2-oxo- ethyl]-2-ethyl-pyrazole- 3-carboxamide | 2.26 | 492.272 |
| 49 | 30 | 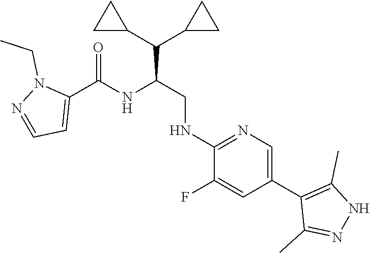 [SMILES: CCn1nccc1C(=O)N[C@H](CNc1ncc(-c2c(C)n[nH]c2C)cc1F)C(C1CC1)C1CC1] | N-[(1S)-1- (dicyclopropylmethyl)-2- [[5-(3,5-dimethyl-1H- pyrazol-4-yl)-3-fluoro-2- pyridyl]amino]-2-oxo- ethyl]-2-ethyl-pyrazole- 3-carboxamide | 2.14 | 480.252 |
| 50 | 30 | 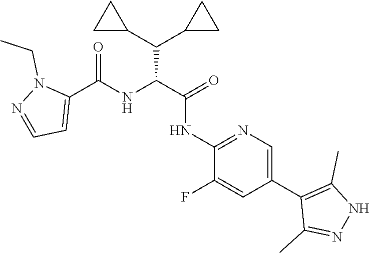 [SMILES: CCn1nccc1C(=O)N[C@@H](C(=O)Nc1ncc(-c2c(C)n[nH]c2C)cc1F)C(C1CC1)C1CC1] | N-[(1R)-1- (dicyclopropylmethyl)-2- [[5-(3,5-dimethyl-1H- pyrazol-4-yl)-3-fluoro-2- pyridyl]amino]-2-oxo- ethyl]-2-ethyl-pyrazole- 3-carboxamide | 2.14 | 480.252 |
| 76 | 171 | 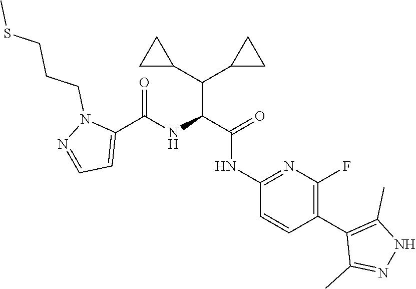 [SMILES: CSCCCn1nccc1C(=O)N[C@H](C(=O)Nc1ccc(-c2c(C)n[nH]c2C)c(F)n1)C(C1CC1)C1CC1] | N-[(1S)-1-(dicycloprop- ylmethyl)-2-[[5-(3,5- dimethyl-1H-pyrazol-4- yl)-6-fluoro-2-pyridyl]- amino]-2-oxo-ethyl]-2- (3-methylsulfanylpropyl) pyrazole-3-carboxamide | 2.37 | 540.256 |
| 77 | 171 | 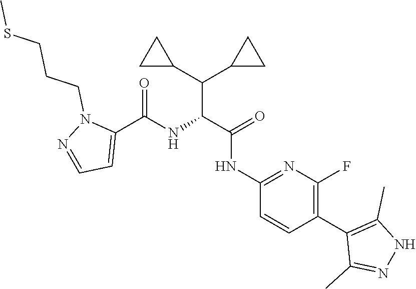 [SMILES: CSCCCn1nccc1C(=O)N[C@@H](C(=O)Nc1ccc(-c2c(C)n[nH]c2C)c(F)n1)C(C1CC1)C1CC1] | N-[(1R)-1-(dicycloprop- ylmethyl)-2-[[5-(3,5- dimethyl-1H-pyrazol-4- yl)-6-fluoro-2-pyridyl]- amino]-2-oxo-ethyl]-2- (3-methylsulfanylpropyl) pyrazole-3-carboxamide | 2.38 | 540.256 |
| 92 | 203 | 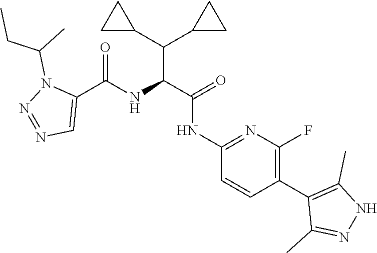 [SMILES: CCC(C)n1nncc1C(=O)N[C@H](C(=O)Nc1ccc(-c2c(C)n[nH]c2C)c(F)n1)C(C1CC1)C1CC1] | N-[(1S)-1-(dicyclo- propylmethyl)-2-[[5- (3,5-dimethyl-1H- pyrazol-4-yl)-6-fluoro-2- pyridyl]amino]-2-oxo- ethyl]-3-sec-butyl- triazole-4-carboxamide; Diastereomer 1 | 2.31 | 509.279 |
| 93 | 203 | 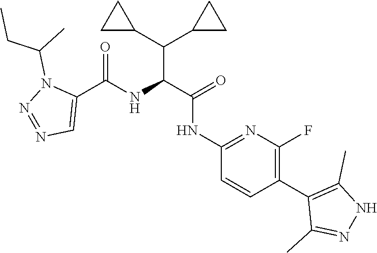 [SMILES: CCC(C)n1nncc1C(=O)N[C@H](C(=O)Nc1ccc(-c2c(C)n[nH]c2C)c(F)n1)C(C1CC1)C1CC1] | N-[(1S)-1-(dicyclo- propylmethyl)-2-[[5- (3,5-dimethyl-1H- pyrazol-4-yl)-6-fluoro-2- pyridyl]amino]-2-oxo- ethyl]-3-sec-butyl- triazole-4-carboxamide; Diastereomer 2 | 2.33 | 509.279 |
| 108 | 230 | 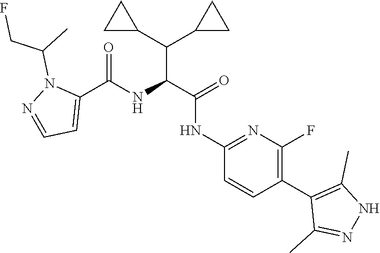 [SMILES: Cc1n[nH]c(C)c1-c1ccc(NC(=O)[C@@H](NC(=O)c2ccnn2C(C)CF)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(2-fluoro-1- methyl-ethyl) pyrazole- 3-carboxamide; Diastereomer 1 | 2.33 | 512.259 |
| 109 | 230 | 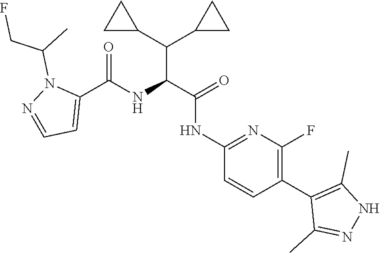 [SMILES: Cc1n[nH]c(C)c1-c1ccc(NC(=O)[C@@H](NC(=O)c2ccnn2C(C)CF)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(2-fluoro-1- methyl-ethyl)pyrazole- 3-carboxamide; Diastereomer 2 | 2.31 | 512.259 |
| 110 | 233 | 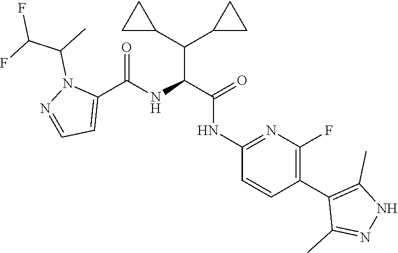 [SMILES: Cc1n[nH]c(C)c1-c1ccc(NC(=O)[C@@H](NC(=O)c2ccnn2C(C)C(F)F)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(2,2-difluoro-1- methyl-ethyl) pyrazole- 3-carboxamide; Diastereomer 1 | 2.37 | 530.249 |
| 111 | 233 | 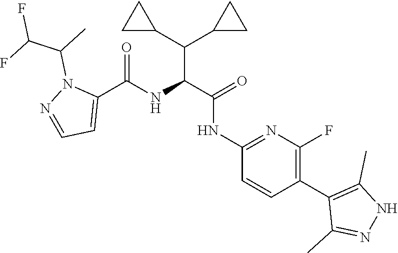 [SMILES: Cc1n[nH]c(C)c1-c1ccc(NC(=O)[C@@H](NC(=O)c2ccnn2C(C)C(F)F)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(2,2-difluoro-1- methyl-ethyl)pyrazole- 3-carboxamide; Diastereomer 2 | 2.39 | 530.249 |
| 162 | 336 | 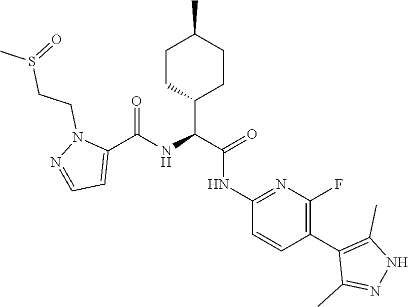 [SMILES: Cc1n[nH]c(C)c1-c1ccc(NC(=O)[C@@H](NC(=O)c2ccnn2CCS(C)=O)[C@H]2CC[C@H](C)CC2)nc1F] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-(2- methylsulfinylethyl) pyrazole-3-carboxamide; Diastereomer 1 | 2.16 | 544.251 |
| 163 | 336 | 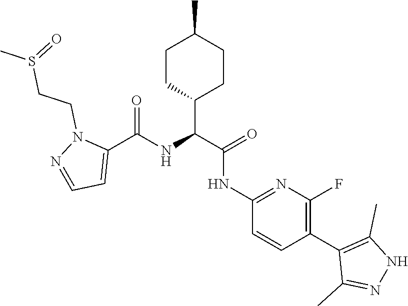 [SMILES: Cc1n[nH]c(C)c1-c1ccc(NC(=O)[C@@H](NC(=O)c2ccnn2CCS(C)=O)[C@H]2CC[C@H](C)CC2)nc1F] | N-[(1S)-2-[[5-(3,5- dimethyl-1H-pyrazol-4- yl)-6-fluoro-2- pyridyl]amino]-1-(4- methylcyclohexyl)-2- oxo-ethyl]-2-(2- methylsulfinylethyl) pyrazole-3-carboxamide; Diastereomer 2 | 2.16 | 544.251 |
| 164 | 337 | 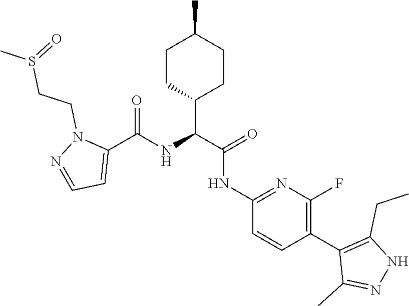 [SMILES: CCc1[nH]nc(C)c1-c1ccc(NC(=O)[C@@H](NC(=O)c2ccnn2CCS(C)=O)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-(2- methylsulfinylethyl) pyrazole-3-carboxamide; Diastereomer 1 | 2.21 | 558.266 |
| 165 | 337 | 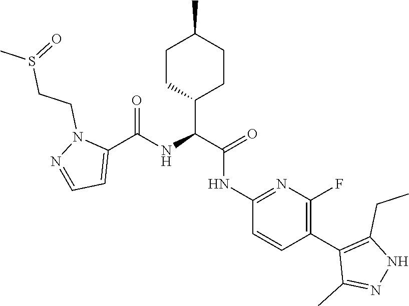 [SMILES: CCc1[nH]nc(C)c1-c1ccc(NC(=O)[C@@H](NC(=O)c2ccnn2CCS(C)=O)[C@H]2CC[C@H](C)CC2)nc1F] | N-[(1S)-2-[[5-(5-ethyl- 3-methyl-1H-pyrazol-4- yl)-6-fluoro-2- pyridyl]amino]-1-(4- methylcyclohexyl)-2- oxo-ethyl]-2-(2- methylsulfinylethyl) pyrazole-3-carboxamide; Diastereomer 2 | 2.21 | 558.266 |
| 177 | 364 | 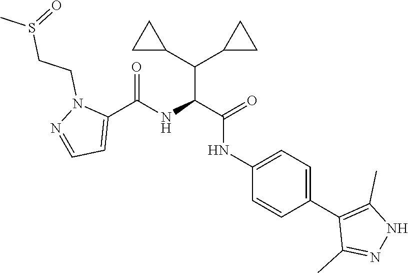 [SMILES: Cc1n[nH]c(C)c1-c1ccc(NC(=O)[C@@H](NC(=O)c2ccnn2CCS(C)=O)C(C2CC2)C2CC2)cc1] | N-[(1S)-1- (dicyclopropylmethyl)-2- [4-(3,5-dimethyl-1H- pyrazol-4-yl)anilino]-2- oxo-ethyl]-2-(2- methylsulfinylethyl) pyrazole-3-carboxamide; Diastereomer 1 | 2.04 | 523.249 |
| 178 | 364 | 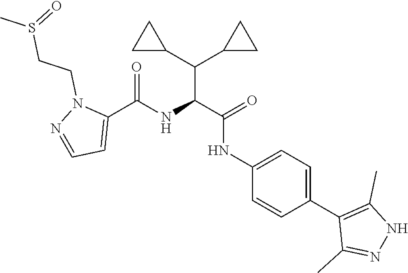 [SMILES: Cc1n[nH]c(C)c1-c1ccc(NC(=O)[C@@H](NC(=O)c2ccnn2CCS(C)=O)C(C2CC2)C2CC2)cc1] | N-[(1S)-1- (dicyclopropylmethyl)-2- [4-(3,5-dimethyl-1H- pyrazol-4-yl)anilino]-2- oxo-ethyl]-2-(2- methylsulfinylethyl) pyrazole-3-carboxamide; Diastereomer 2 | 2.04 | 523.249 |
| 180 | 366 | 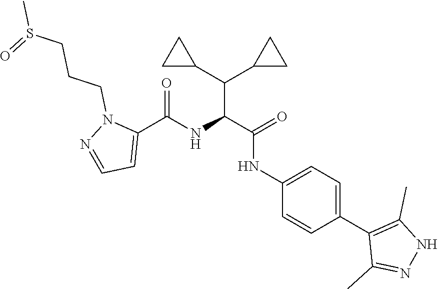 [SMILES: Cc1n[nH]c(C)c1-c1ccc(NC(=O)[C@@H](NC(=O)c2ccnn2CCCS(C)=O)C(C2CC2)C2CC2)cc1] | Diastereomer 1 N-[(1S)- 1-(dicyclopropylmethyl)- 2-[4-(3,5-dimethyl-1H- pyrazol-4-yl)anilino]-2- oxo-ethyl]-2-(3- methylsulfinylpropyl) pyrazole-3-carboxamide | 2.03 | 537.265 |
| 181 | 366 | 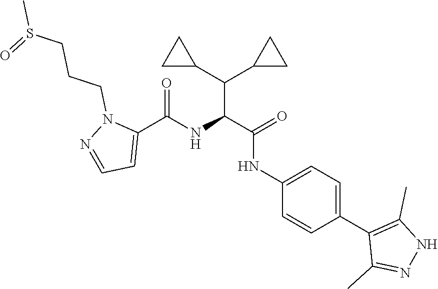 [SMILES: Cc1n[nH]c(C)c1-c1ccc(NC(=O)[C@@H](NC(=O)c2ccnn2CCCS(C)=O)C(C2CC2)C2CC2)cc1] | Diastereomer 2 N-[(1S)- 1-(dicyclopropylmethyl)- 2-[4-(3,5-dimethyl-1H- pyrazol-4-yl)anilino]-2- oxo-ethyl]-2-(3- methylsulfinylpropyl) pyrazole-3-carboxamide | 2.04 | 537.265 |
| 183 | 370 | 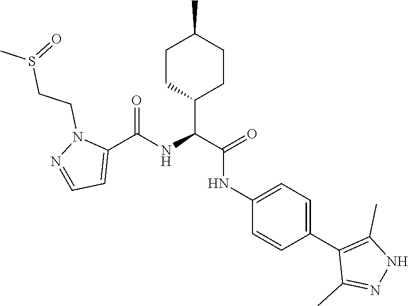 [SMILES: Cc1n[nH]c(C)c1-c1ccc(NC(=O)[C@@H](NC(=O)c2ccnn2CCS(C)=O)[C@H]2CC[C@H](C)CC2)cc1] | N-[(1S)-2-[4-(3,5- dimethyl-1H-pyrazol-4- yl)anilino]-1-(4- methylcyclohexyl)-2- oxo-ethyl]-2-(2- methylsulfinylethyl) pyrazole-3-carboxamide Diastereomer 1 | 2.12 | 525.265 |
| 184 | 370 | 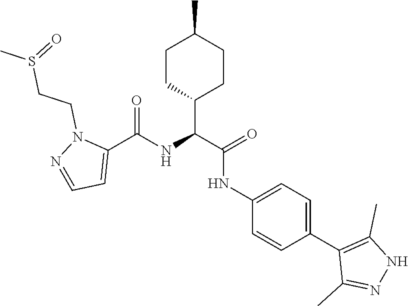 [SMILES: Cc1n[nH]c(C)c1-c1ccc(NC(=O)[C@@H](NC(=O)c2ccnn2CCS(C)=O)[C@H]2CC[C@H](C)CC2)cc1] | N-[(1S)-2-[4-(3,5- dimethyl-1H-pyrazol-4- yl)anilino]-1-(4- methylcyclohexyl)-2- oxo-ethyl]-2-(2- methylsulfinylethyl) pyrazole-3-carboxamide; Diastereomer 2 | 2.12 | 525.265 |
| 186 | 372 | 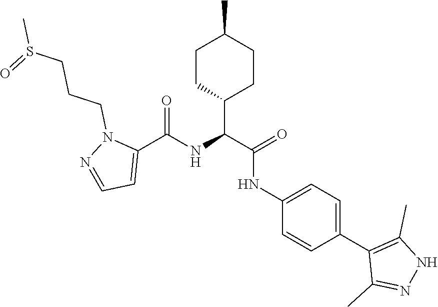 [SMILES: Cc1n[nH]c(C)c1-c1ccc(NC(=O)[C@@H](NC(=O)c2ccnn2CCCS(C)=O)[C@H]2CC[C@H](C)CC2)cc1] | N-[(1S)-2-[4-(3,5- dimethyl-1H-pyrazol-4- yl)anilino]-1-(4- methylcyclohexyl)-2- oxo-ethyl]-2-(3- methylsulfinylpropyl) pyrazole-3-carboxamide; Diastereomer 1 | 2.12 | 539.280 |
| 187 | 372 | 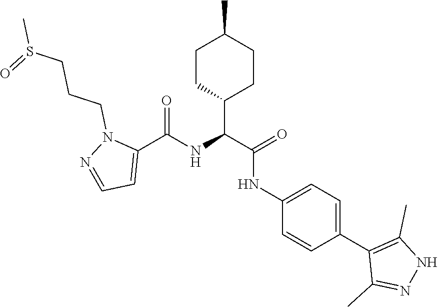 [SMILES: Cc1n[nH]c(C)c1-c1ccc(NC(=O)[C@@H](NC(=O)c2ccnn2CCCS(C)=O)[C@H]2CC[C@H](C)CC2)cc1] | N-[(1S)-2-[4-(3,5- dimethyl-1H-pyrazol-4- yl)anilino]-1-(4- methylcyclohexyl)-2- oxo-ethyl]-2-(3- methylsulfinylpropyl) pyrazole-3-carboxamide; Diastereomer 2 | 2.12 | 539.281 |

### Example 51: N-[(1S)-1-(dicyclopropylmethyl)-2-[[5-(3,5-dimethyl-1H-pyrazol-4-yl)pyrazin-2-yl]amino]-2-oxo-ethyl]-2-ethyl-pyrazole-3-carboxamide

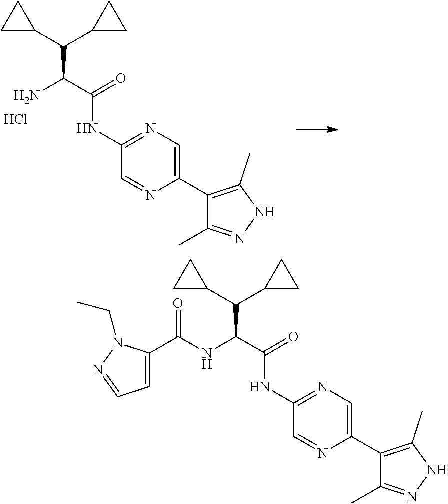

HATU (32.8 mg, 0.086 mmol) was added to a solution of the compound of Preparation 67 (38.0 mg, 0.086 mmol), 2-ethylpyrazole-3-carboxylic acid (12.1 mg, 0.086 mmol) and DIPEA (0.075 mL, 0.431 mmol) in DMF (1 mL) and the reaction mixture was stirred at room temperature for 1 hour. The reaction mixture was purified directly by prep. basic HPLC to afford the title compound as a colourless solid (21.7 mg, 54% yield). 1H NMR (400 MHz, DMSO-d6) δ 10.96 (s, 1H), 9.33 (d, J=1.6 Hz, 1H), 8.52 (d, J=8.6 Hz, 1H), 8.46 (d, J=1.6 Hz, 1H), 7.50 (d, J=2.0 Hz, 1H), 7.03 (d, J=2.1 Hz, 1H), 4.99 (t, J=8.0 Hz, 1H), 4.48 (qd, J=7.1, 2.5 Hz, 2H), 2.36 (s, 6H), 1.29 (t, J=7.1 Hz, 3H), 1.04-0.92 (m, 1H), 0.93-0.74 (m, 2H), 0.49 (dt, J=7.5, 5.2 Hz, 1H), 0.46-0.27 (m, 3H), 0.27-0.11 (m, 4H); LCMS (ES): m/z 463.256 [M+H]⁺; RT=2.15 min.

The examples listed in the table below were all accessed using the method described for Example 51, reacting the indicated amine with the appropriate carboxylic acid.

| | Precursor | | | | |
|---|---|---|---|---|---|
| Ex. | Prep. | | | LCMS | Mass |
| No. | number | Structure | Name | RT | ion |
| 52 | 72 | 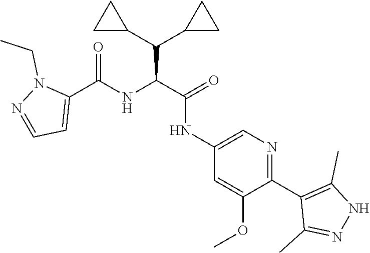 [SMILES: CCn1nccc1C(=O)N[C@H](C(=O)Nc1cnc(-c2c(C)n[nH]c2C)c(OC)c1)C(C1CC1)C1CC1] | N-[(1S)-1- (dicyclopropylmethyl)-2- [[6-(3,5-dimethyl-1H- pyrazol-4-yl)-5- methoxy-3- pyridyl]amino]-2-oxo- ethyl]-2-ethyl-pyrazole- 3-carboxamide | 2.06 | 492.272 |
| 53 | 71 | 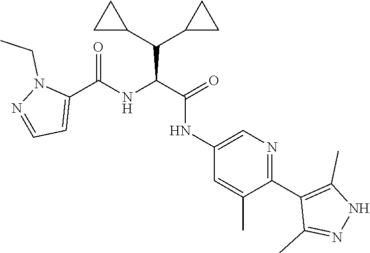 [SMILES: CCn1nccc1C(=O)N[C@H](C(=O)Nc1cnc(-c2c(C)n[nH]c2C)c(C)c1)C(C1CC1)C1CC1] | N-[(1S)-1- (dicyclopropylmethyl)-2- [[6-(3,5-dimethyl-1H- pyrazol-4-yl)-5-methyl- 3-pyridyl]amino]-2-oxo- ethyl]-2-ethyl-pyrazole- 3-carboxamide | 2.00 | 476.277 |
| 54 | 70 | 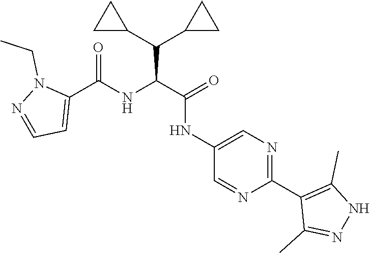 [SMILES: CCn1nccc1C(=O)N[C@H](C(=O)Nc1cnc(-c2c(C)n[nH]c2C)nc1)C(C1CC1)C1CC1] | N-[(1S)-1- (dicyclopropylmethyl)-2- [[2-(3,5-dimethyl-1H- pyrazol-4-yl)pyrimidin- 5-yl]amino]-2-oxo- ethyl]-2-ethyl-pyrazole- 3-carboxamide | 2.14 | 463.257 |
| 55 | 68 | 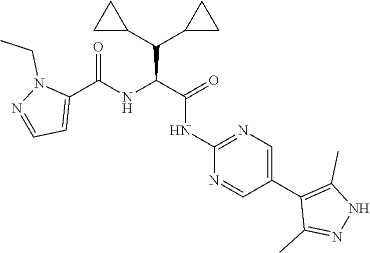 [SMILES: CCn1nccc1C(=O)N[C@H](C(=O)Nc1ncc(-c2c(C)n[nH]c2C)cn1)C(C1CC1)C1CC1] | N-[(1S)-1- (dicyclopropylmethyl)-2- [[5-(3,5-dimethyl-1H- pyrazol-4-yl)pyrimidin- 2-yl]amino]-2-oxo- ethyl]-2-ethyl-pyrazole- 3-carboxamide | 2.05 | 463.257 |
| 56 | 69 | 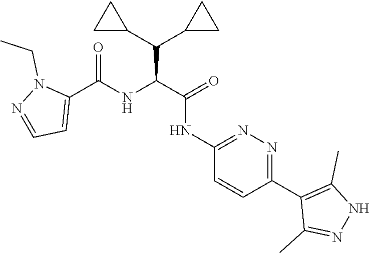 [SMILES: CCn1nccc1C(=O)N[C@H](C(=O)Nc1ccc(-c2c(C)n[nH]c2C)nn1)C(C1CC1)C1CC1] | N-[(1S)-1- (dicyclopropylmethyl)-2- [[6-(3,5-dimethyl-1H- pyrazol-4-yl)pyridazin- 3-yl]amino]-2-oxo- ethyl]-2-ethyl-pyrazole- 3-carboxamide | 2.11 | 463.257 |
| 87 | 183 | 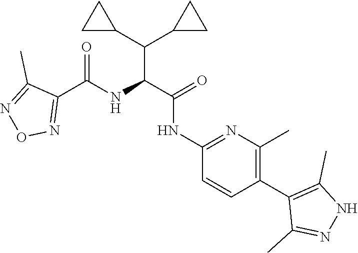 [SMILES: Cc1nc(NC(=O)[C@@H](NC(=O)c2nonc2C)C(C2CC2)C2CC2)ccc1-c1c(C)n[nH]c1C] | N-[(1S)-1- (dicyclopropylmethyl)-2- [[5-(3,5-dimethyl-1H- pyrazol-4-yl)-6-fluoro-2- pyridyl]amino]-2-oxo- ethyl]-4-methyl-1,2,5- oxadiazole-3- carboxamide | 2.40 | 468.218 |
| 88 | 183 | 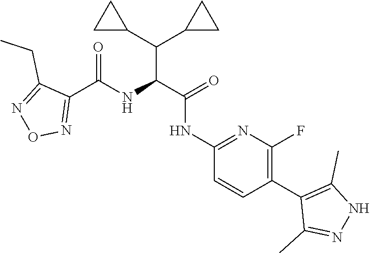 [SMILES: CCc1nonc1C(=O)N[C@H](C(=O)Nc1ccc(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-6-fluoro-2- pyridyl]amino]-2-oxo- ethyl]-4-ethyl-1,2,5- oxadiazole-3- carboxamide | 2.49 | 482.232 |
| 96 | 183 | 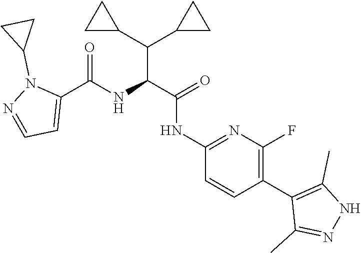 [SMILES: Cc1n[nH]c(C)c1-c1ccc(NC(=O)[C@@H](NC(=O)c2ccnn2C2CC2)C(C2CC2)C2CC2)nc1F] | 2-cyclopropyl-N-[(1S)- 1-(dicyclopropylmethyl)- 2-[[5-(3,5-dimethyl-1H- pyrazol-4-yl)-6-fluoro-2- pyridyl]amino]-2-oxo- ethyl]pyrazole-3- carboxamide | 2.29 | 492.253 |
| 97 | 183 | 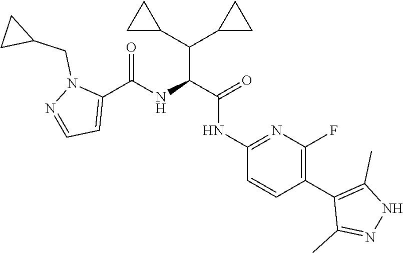 [SMILES: Cc1n[nH]c(C)c1-c1ccc(NC(=O)[C@@H](NC(=O)c2ccnn2CC2CC2)C(C2CC2)C2CC2)nc1F] | 2-(cyclopropylmethyl)- N-[(1S)-1- (dicyclopropylmethyl)-2- [[5-(3,5-dimethyl-1H- pyrazol-4-yl)-6-fluoro-2- pyridyl]amino]-2-oxo- ethyl]pyrazole-3- carboxamide | 2.35 | 506.268 |
| 98 | 183 | 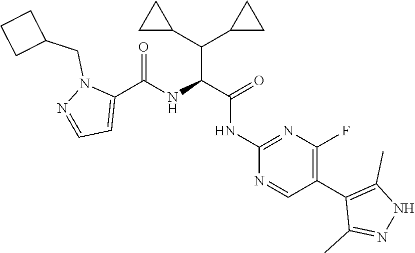 [SMILES: Cc1n[nH]c(C)c1-c1cnc(NC(=O)[C@@H](NC(=O)c2ccnn2CC2CCC2)C(C2CC2)C2CC2)nc1F] | 2-(cyclobutylmethyl)-N- [(1S)-1- (dicyclopropylmethyl)-2- [[5-(3,5-dimethyl-1H- pyrazol-4-yl)-6-fluoro-2- pyridyl]amino]-2-oxo- ethyl]pyrazole-3- carboxamide | 2.43 | 520.284 |
| 99 | 183 | 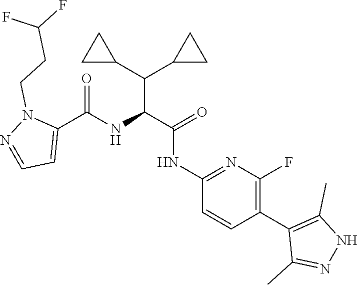 [SMILES: Cc1n[nH]c(C)c1-c1ccc(NC(=O)[C@@H](NC(=O)c2ccnn2CCC(F)F)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-(3,3- difluoropropyl)pyrazole- 3-carboxamide | 2.34 | 530.249 |
| 101 | 183 | 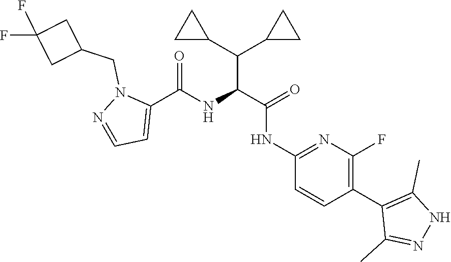 [SMILES: Cc1n[nH]c(C)c1-c1ccc(NC(=O)[C@@H](NC(=O)c2ccnn2CC2CC(F)(F)C2)C(C2CC2)C2CC2)nc1F] | N-[(1S)-1-(dicyclo- propylmethyl)-2-[[5- (3,5-dimethyl-1H- pyrazol-4-yl)-6-fluoro-2- pyridyl]amino]-2-oxo- ethyl]-2-[(3,3-difluoro- cyclobutyl)methyl] pyrazole-3-carboxamide | 2.41 | 556.265 |
| 102 | 183 | 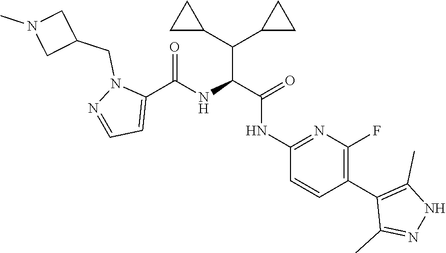 [SMILES: Cc1n[nH]c(C)c1-c1ccc(NC(=O)[C@@H](NC(=O)c2ccnn2CC2CN(C)C2)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-2-[(1- methylazetidin-3- yl)methyl]pyrazole-3- carboxamide | 1.96 | 535.295 |
| 106 | 183 | 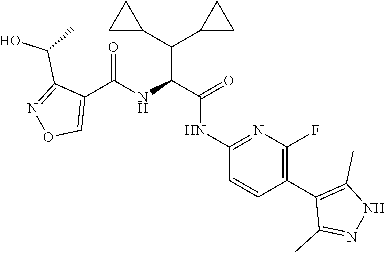 [SMILES: Cc1n[nH]c(C)c1-c1ccc(NC(=O)[C@@H](NC(=O)c2conc2[C@@H](C)O)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-3-[(1R)-1- hydroxyethyl]isoxazole- 4-carboxamide | 2.19 | 497.231 |
| 107 | 183 | 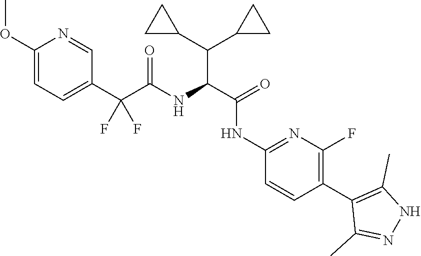 [SMILES: COc1ccc(C(F)(F)C(=O)N[C@H](C(=O)Nc2ccc(-c3c(C)n[nH]c3C)c(F)n2)C(C2CC2)C2CC2)cn1] | (2S)-3,3-dicyclopropyl- 2-[[2,2-difluoro-2-(6- methoxy-3- pyridyl)acetyl]amino]-N- [5-(3,5-dimethyl-1H- pyrazol-4-yl)-6-fluoro-2- pyridyl]propanamide | 2.43 | 543.233 |
| 116 | 241 | 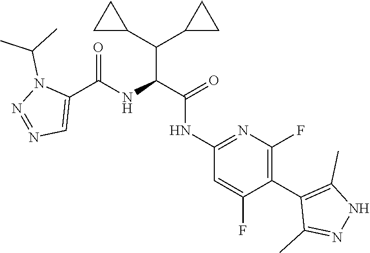 [SMILES: Cc1n[nH]c(C)c1-c1c(F)cc(NC(=O)[C@@H](NC(=O)c2cnnn2C(C)C)C(C2CC2)C2CC2)nc1F] | N-[(1S)-1- (dicyclopropylmethyl)-2- [[5-(3,5-dimethyl-1H- pyrazol-4-yl)-4,6- difluoro-2- pyridyl]amino]-2-oxo- ethyl]-3-isopropyl- triazole-4-carboxamide | 2.29 | 513.254 |
| 117 | 241 | 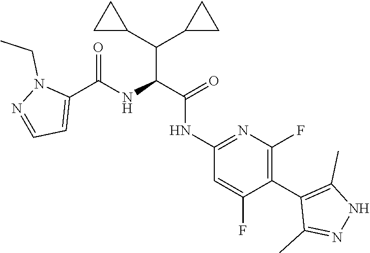 [SMILES: CCn1nccc1C(=O)N[C@H](C(=O)Nc1cc(F)c(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-4,6- difluoro-2- pyridyl]amino]-2-oxo- ethyl]-2-ethyl-pyrazole- 3-carboxamide | 2.31 | 498.243 |
| 118 | 241 | 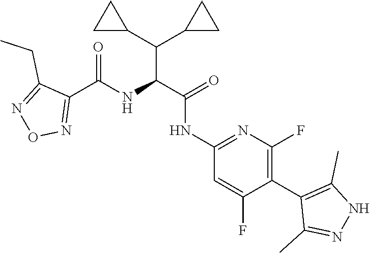 [SMILES: CCc1nonc1C(=O)N[C@H](C(=O)Nc1cc(F)c(-c2c(C)n[nH]c2C)c(F)n1)C(C1CC1)C1CC1] | N-[(1S)-1- (dicyclopropylmethyl)-2- [[5-(3,5-dimethyl-1H- pyrazol-4-yl)-4,6- difluoro-2- pyridyl]amino]-2-oxo- ethyl]-4-ethyl-1,2,5- oxadiazole-3- carboxamide | 2.52 | 500.222 |
| 119 | 241 | 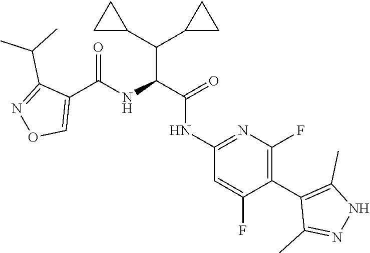 [SMILES: Cc1n[nH]c(C)c1-c1c(F)cc(NC(=O)[C@@H](NC(=O)c2conc2C(C)C)C(C2CC2)C2CC2)nc1F] | N-[(1S)-1- (dicyclopropylmethyl)-2- [[5-(3,5-dimethyl-1H- pyrazol-4-yl)-4,6- difluoro-2- pyridyl]amino]-2-oxo- ethyl]-3-isopropyl- isoxazole-4-carboxamide | 2.41 | 513.243 |
| 131 | 281 | 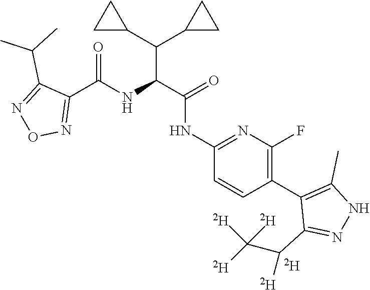 | N-[(1S)-1- (dicyclopropylmethyl)-2- [[6-fluoro-5-[5-methyl- 3-(1,1,2,2,2- pentadeuterioethyl)-1H- pyrazol-4-yl]-2- pyridyl]amino]-2-oxo- ethyl]-3-isopropyl- isoxazole-4- carboxamide | 2.42 | 514.299 |
| 132 | 281 | 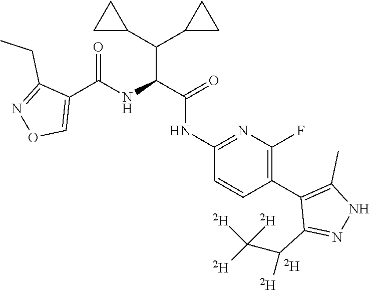 | N-[(1S)-1- (dicyclopropylmethyl)-2- [[6-fluoro-5-[5-methyl- 3-(1,1,2,2,2- pentadeuterioethyl)-1H- pyrazol-4-yl]-2- pyridyl]amino]-2-oxo- ethyl]-3-ethyl-isoxazole- 4-carboxamide | 2.36 | 500.283 |
| 133 | 281 | 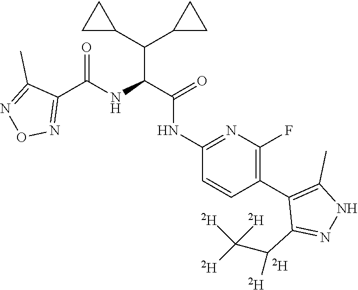 | N-[(1S)-1- (dicyclopropylmethyl)-2- [[6-fluoro-5-[5-methyl- 3-(1,1,2,2,2- pentadeuterioethyl)-1H- pyrazol-4-yl]-2- pyridyl]amino]-2-oxo- ethyl]-3-methyl- isoxazole-4- carboxamide | 2.29 | 486.268 |
| 134 | 281 | 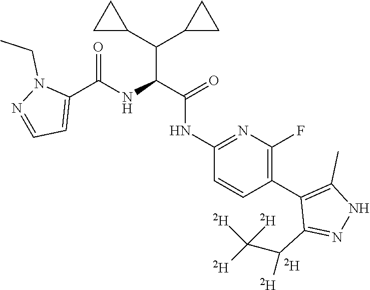 | N-[(1S)-1- (dicyclopropylmethyl)-2- [[6-fluoro-5-[5-methyl- 3-(1,1,2,2,2- pentadeuterioethyl)-1H- pyrazol-4-yl]-2- pyridyl]amino]-2-oxo- ethyl]-2-ethyl-pyrazole- 3-carboxamide | 2.32 | 500.283 |
| 135 | 281 | 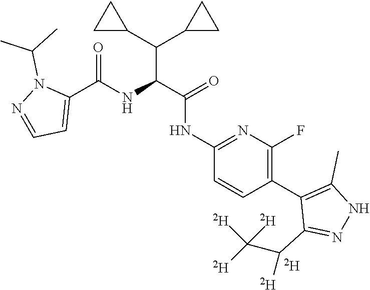 | N-[(1S)-1- (dicyclopropylmethyl)-2- [[6-fluoro-5-[5-methyl- 3-(1,1,2,2,2- pentadeuterioethyl)-1H- pyrazol-4-yl]-2- pyridyl]amino]-2-oxo- ethyl]-2-isopropyl- pyrazole-3-carboxamide | 2.39 | 513.315 |
| 136 | 281 | 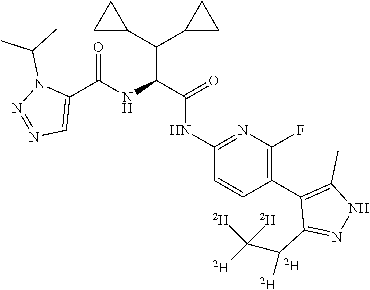 | N-[(1S)-1- (dicyclopropylmethyl)-2- [[6-fluoro-5-[5-methyl- 3-(1,1,2,2,2- pentadeuterioethyl)-1H- pyrazol-4-yl]-2- pyridyl]amino]-2-oxo- ethyl]-3-isopropyl- triazole-4-carboxamide | 2.30 | 514.31 |
| 168 | 183 | 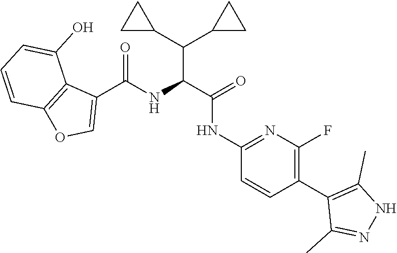 [SMILES: Cc1n[nH]c(C)c1-c1ccc(NC(=O)[C@@H](NC(=O)c2coc3cccc(O)c23)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-4-hydroxy- benzofuran-3- carboxamide | 2.48 | 518.221 |
| 169 | 183 | 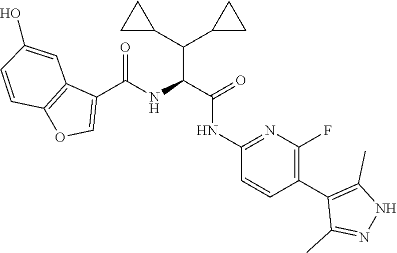 [SMILES: Cc1n[nH]c(C)c1-c1ccc(NC(=O)[C@@H](NC(=O)c2coc3ccc(O)cc23)C(C2CC2)C2CC2)nc1F] | N-[(1S)-1- (dicyclopropylmethyl)-2- [[5-(3,5-dimethyl-1H- pyrazol-4-yl)-6-fluoro-2- pyridyl]amino]-2-oxo- ethyl]-5-hydroxy- benzofuran-3- carboxamide | 2.27 | 518.221 |
| 173 | 360 | 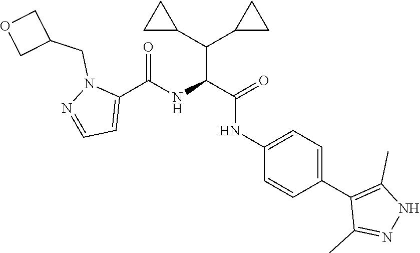 [SMILES: Cc1n[nH]c(C)c1-c1ccc(NC(=O)[C@@H](NC(=O)c2ccnn2CC2COC2)C(C2CC2)C2CC2)cc1] | N-[(1S)-1- (dicyclopropylmethyl)-2- [4-(3,5-dimethyl-1H- pyrazol-4-yl)anilino]-2- oxo-ethyl]-2-(oxetan-3- ylmethyl)pyrazole-3- carboxamide | 2.13 | 503.277 |

### Example 57: N-[(1S)-1-cyclohexyl-2-[[6-(3,5-dimethyl-1H-pyrazol-4-yl)-3-pyridyl]amino]-2-oxo-ethyl]-2-isopropyl-pyrazole-3-carboxamide

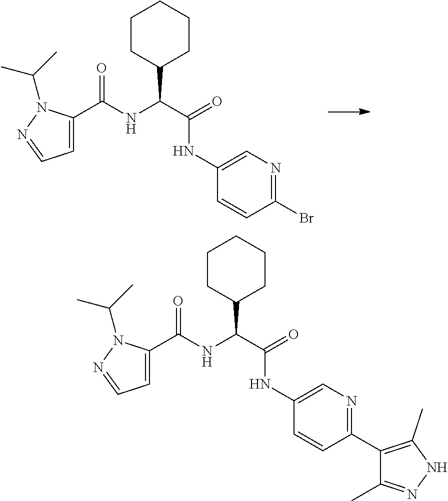

K₂CO₃ (37.0 mg, 0.268 mmol) was added to a solution of the compound of Preparation 125 (30.0 mg, 0.067 mmol) and 3,5-dimethyl-4-(4,4,5,5-tetramethyl-1,3,2-dioxaborolan-2-yl)-1H-pyrazole (29.7 mg, 0.134 mmol) in DMF:water (1.8 mL:0.6 mL). The reaction mixture was degassed with nitrogen for 10 minutes. Pd(dppf)Cl₂·DCM (10.9 mg, 0.0134 mmol) was added and the sealed reaction mixture was stirred at 100° C. for 1 hour. The reaction mixture was filtered through a PTFE filter and purified directly by prep. basic HPLC. The obtained slightly impure compound was purified by silica column chromatography (230-400 mesh), eluting with MeOH (0-20%) in DCM, to afford the title compound as a colourless solid (16 mg, 51% yield). 1H NMR (400 MHz, DMSO-d6) δ 12.32 (s, 1H), 10.40 (s, 1H), 8.80 (d, J=2.6 Hz, 1H), 8.53 (d, J=8.1 Hz, 1H), 8.08 (dd, J=8.6, 2.6 Hz, 1H), 7.50 (d, J=1.9 Hz, 1H), 7.37 (d, J=8.6 Hz, 1H), 6.97 (d, J=2.0 Hz, 1H), 5.39 (h, J=6.6 Hz, 1H), 4.41 (t, J=8.5 Hz, 1H), 2.31 (s, 6H), 1.85 (d, J=14.2 Hz, 2H), 1.72 (s, 2H), 1.63 (d, J=9.7 Hz, 2H), 1.36 (dd, J=8.4, 6.6 Hz, 6H), 1.29-0.96 (m, 5H); LCMS (ES): m/z 464.278 [M+H]⁺; RT=2.13 min.

The examples listed in the table below were all accessed using the method described for Example 57.

| | Precursor | | | | |
|---|---|---|---|---|---|
| Ex. | Prep. | | | LCMS | Mass |
| no. | number | Structure | Name | RT | ion |
| 58 | 132 | 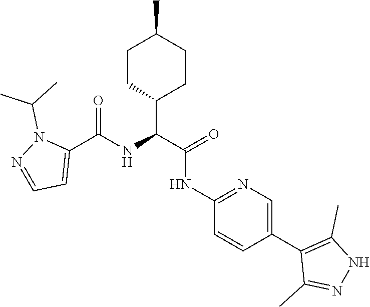 [SMILES: Cc1n[nH]c(C)c1-c1ccc(NC(=O)[C@@H](NC(=O)c2ccnn2C(C)C)[C@H]2CC[C@H](C)CC2)nc1] | N-[(1S)-2-[[5-(3,5- dimethyl-1H-pyrazol-4- yl)-2-pyridyl]amino]-1- (4-methylcyclohexyl)-2- oxo-ethyl]-2-isopropyl- pyrazole-3-carboxamide | 2.35 | 478.293 |
| 59 | 122 | 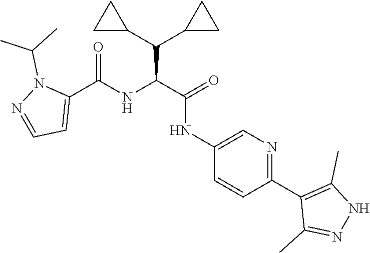 [SMILES: Cc1n[nH]c(C)c1-c1ccc(NC(=O)[C@@H](NC(=O)c2ccnn2C(C)C)C(C2CC2)C2CC2)cn1] | N-[(1S)-1- (dicyclopropylmethyl)-2- [[6-(3,5-dimethyl-1H- pyrazol-4-yl)-3- pyridyl]amino]-2-oxo- ethyl]-2-isopropyl- pyrazole-3-carboxamide | 2.11 | 476.277 |
| 60 | 128 | 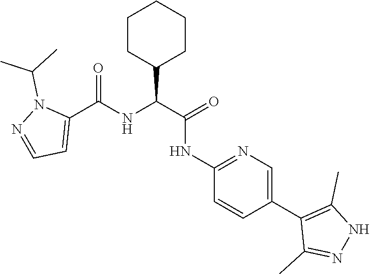 [SMILES: Cc1n[nH]c(C)c1-c1ccc(NC(=O)[C@@H](NC(=O)c2ccnn2C(C)C)C2CCCCC2)nc1] | N-[(1S)-1-cyclohexyl-2- [[5-(3,5-dimethyl-1H- pyrazol-4-yl)-2- pyridyl]amino]-2-oxo- ethyl]-2-isopropyl- pyrazole-3-carboxamide | 2.26 | 464.278 |

### Example 61: N-[(1S)-2-[[6-(3,5-dimethyl-1H-pyrazol-4-yl)-3-pyridyl]amino]-1-((1r,4S)-4-methylcyclohexyl)-2-oxo-ethyl]-2-isopropyl-pyrazole-3-carboxamide and Example 62: N-[(1R)-2-[[6-(3,5-dimethyl-1H-pyrazol-4-yl)-3-pyridyl]amino]-1-((1r,4S)-4-methylcyclohexyl)-2-oxo-ethyl]-2-isopropyl-pyrazole-3-carboxamide

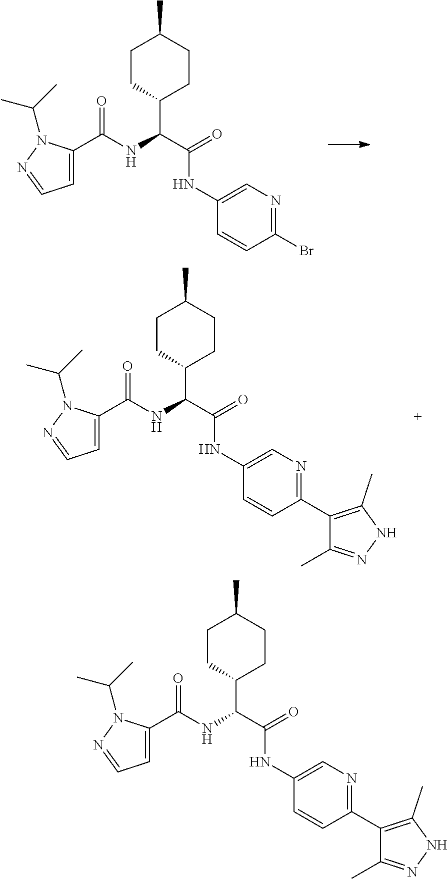

K₂CO₃ (119 mg, 0.86 mmol) was added to a solution of the compound of Preparation 135 (100 mg, 0.21 mmol) and 3,5-dimethyl-4-(4,4,5,5-tetramethyl-1,3,2-dioxaborolan-2-yl)-1H-pyrazole (96 mg, 0.43 mmol) in DMF:water (1.8 mL:0.2 mL). The reaction mixture was degassed with nitrogen for 10 minutes. Pd(dppf)Cl₂·DCM (35 mg, 0.04 mmol) was added and the sealed reaction mixture was stirred under microwave conditions at 120° C. for 2 hours. The reaction mixture was filtered through Celite™ and washed with EtOAc (10 mL). The filtrate was separated and the organic layer was dried over Na₂SO₄, filtered and concentrated in vacuo. The crude product was purified by prep. basic HPLC to afford the title compounds as a racemic mixture (60 mg, 58% yield). This was dissolved in MeOH (1 mL) and separated by SFC (IC column, 30% MeOH, isocratic run) to afford the title compounds as colourless solids.

Example 61: Peak 1 (retention time 4.31 min, 20.0 mg, 19% yield); 1H NMR (400 MHz, DMSO-d6) δ 12.33-12.13 (m, 1H), 10.53 (s, 1H), 8.81 (d, J=2.27 Hz, 1H), 8.70 (br d, J=7.75 Hz, 1H), 8.09 (dd, J=8.70, 2.62 Hz, 1H), 7.49 (d, J=1.91 Hz, 1H), 7.36 (d, J=8.34 Hz, 1H), 6.99 (d, J=2.03 Hz, 1H), 5.40 (quin, J=6.59 Hz, 1H), 4.37 (br t, J=8.34 Hz, 1H), 3.27 (d, J=10.25 Hz, 1H), 2.35-2.26 (m, 6H), 1.92-1.76 (m, 2H), 1.70 (br d, J=12.28 Hz, 2H), 1.65-1.57 (m, 1H), 1.35 (dd, J=8.46, 6.56 Hz, 6H), 1.28-1.12 (m, 1H), 1.11-1.00 (m, 1H), 0.96-0.79 (m, 5H); LCMS (ES): m/z 478.292 [M+H]⁺; RT=2.23 min.

Example 62: Peak 2 (retention time 5.77 min, 16.0 mg, 17% yield); 1H NMR (400 MHz, DMSO-d6) δ 12.34-12.11 (m, 1H), 10.53 (s, 1H), 8.81 (d, J=2.27 Hz, 1H), 8.70 (br d, J=7.75 Hz, 1H), 8.09 (dd, J=8.70, 2.62 Hz, 1H), 7.49 (d, J=1.91 Hz, 1H), 7.36 (d, J=8.34 Hz, 1H), 6.99 (d, J=2.03 Hz, 1H), 5.40 (quin, J=6.59 Hz, 1H), 4.37 (t, J=8.34 Hz, 1H), 3.27 (d, J=10.25 Hz, 1H), 2.35-2.24 (m, 6H), 1.92-1.76 (m, 2H), 1.70 (d, J=12.28 Hz, 2H), 1.63-1.55 (m, 1H), 1.35 (dd, J=8.46, 6.56 Hz, 6H), 1.28-1.12 (m, 1H), 1.11-1.00 (m, 1H), 0.97-0.68 (m, 5H); LCMS (ES): m/z 478.292 [M+H]⁺; RT=2.23 min.

The examples listed in the table below were all accessed using the method described for Example 61 and Example 62.

| | Precursor | | | | |
|---|---|---|---|---|---|
| Ex. | Prep. | | | LCMS | Mass |
| no. | number | Structure | Name | RT | ion |
| 63 | 120 | 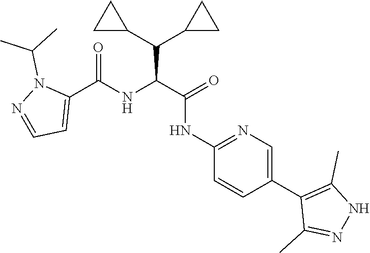 [SMILES: Cc1n[nH]c(C)c1-c1ccc(NC(=O)[C@@H](NC(=O)c2ccnn2C(C)C)C(C2CC2)C2CC2)nc1] | N-[(1S)-1- (dicyclopropylmethyl)-2- [[5-(3,5-dimethyl-1H- pyrazol-4-yl)-2- pyridyl]amino]-2-oxo- ethyl]-2-isopropyl- pyrazole-3-carboxamide | 2.35 | 478.293 |
| 64 | 120 | 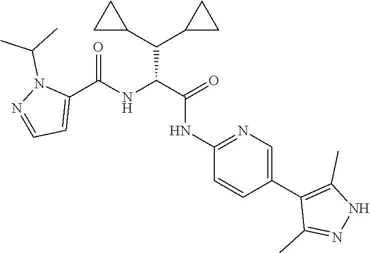 [SMILES: Cc1n[nH]c(C)c1-c1ccc(NC(=O)[C@H](NC(=O)c2ccnn2C(C)C)C(C2CC2)C2CC2)nc1] | N-[(1R)-1- (dicyclopropylmethyl)-2- [[5-(3,5-dimethyl-1H- pyrazol-4-yl)-2- pyridyl]amino]-2-oxo- ethyl]-2-isopropyl- pyrazole-3-carboxamide | 2.11 | 476.277 |

### Example 89: N-[(1S)-1-(dicyclopropylmethyl)-2-[[5-(3,5-dimethyl-1H-pyrazol-4-yl)-6-fluoro-2-pyridyl]amino]-2-oxo-ethyl]-4-(3-hydroxypropyl)-1,2,5-oxadiazole-3-carboxamide

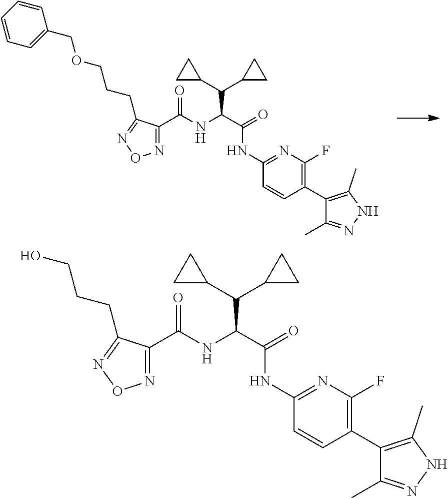

Triethylsilane (0.07 mL, 0.44 mmol) was added to a solution of the compound of Preparation 190 (66.0 mg, 0.11 mmol) and 10% Pd/C (100 mg) in degassed MeOH (15 mL). The reaction mixture was stirred at room temperature for 45 minutes. The reaction mixture was filtered and purified directly by prep. basic HPLC to afford the title compound as a colourless solid (5.6 mg, 10% yield). 1H NMR (400 MHz, DMSO-d6) δ 12.41 (s, 1H), 10.96 (s, 1H), 9.16 (d, J=8.6 Hz, 1H), 8.12-7.96 (m, 1H), 7.88 (dd, J=10.1, 8.1 Hz, 1H), 4.96 (t, J=7.5 Hz, 1H), 4.55 (t, J=5.1 Hz, 1H), 3.44 (q, J=5.9 Hz, 2H), 2.94 (dd, J=8.5, 6.7 Hz, 2H), 2.10 (s, 6H), 1.82 (p, J=6.7 Hz, 2H), 1.01-0.72 (m, 3H), 0.56-0.13 (m, 9H); LCMS (ES): m/z 512.242 [M+H]⁺; RT=2.26 min.

### Example 105: N-[(1S)-1-(dicyclopropylmethyl)-2-[[5-(3,5-dimethyl-1H-pyrazol-4-yl)-6-fluoro-2-pyridyl]amino]-2-oxo-ethyl]-4-fluoro-2-(3-hydroxypropyl)pyrazole-3-carboxamide

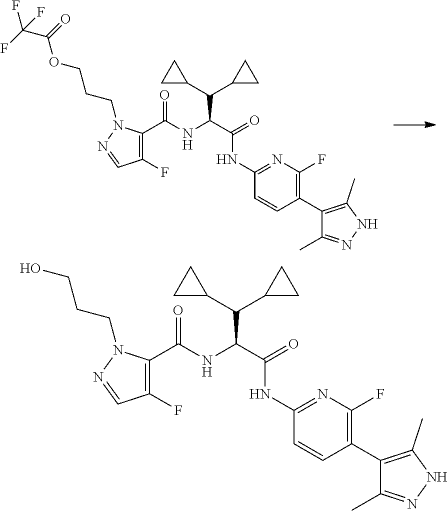

K₂CO₃ (excess) was added to a solution of the compound of Preparation 227 (32 mg, 0.05 mmol) in MeOH (1.0 mL) and stirred at room temperature for 1 hour. The reaction mixture was filtered and purified directly by prep. acidic HPLC to afford the title compound as a colourless solid (16.7 mg, 63% yield). 1H NMR (400 MHz, DMSO-d6) δ 12.42 (s, 1H), 10.92 (s, 1H), 8.24 (dd, J=8.6, 2.6 Hz, 1H), 8.04 (dd, J=8.2, 1.9 Hz, 1H), 7.88 (dd, J=10.1, 8.1 Hz, 1H), 7.63 (d, J=4.5 Hz, 1H), 4.89 (dd, J=8.5, 5.6 Hz, 1H), 4.52 (s, 1H), 4.46-4.26 (m, 2H), 3.38-3.34 (m, 2H), 2.11 (s, 6H), 1.85 (p, J=6.8 Hz, 2H), 0.99-0.72 (m, 3H), 0.55-0.12 (m, 8H); LCMS (ES): m/z 528.254 [M+H]⁺; RT=2.23 min.

### Example 122: N-[(1S)-1-(dicyclopropylmethyl)-2-[[6-(difluoromethyl)-5-(3,5-dimethyl-1H-pyrazol-4-yl)-2-pyridyl]amino]-2-oxo-ethyl]-2-ethyl-pyrazole-3-carboxamide

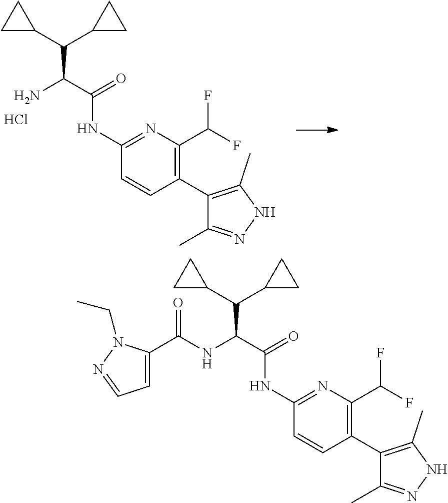

HATU (20.0 mg, 0.053 mmol) was added to a solution of the compound of Preparation 253 (22.0 mg, 0.026 mmol), 2-ethylpyrazole-3-carboxylic acid (8.0 mg, 0.057 mmol) and DIPEA (0.05 mL, 0.28 mmol) in DMF (1 mL) and the reaction mixture was stirred at room temperature for 3 hours. The reaction mixture was diluted with MeOH (1 mL) and K₂CO₃ (5 mg) was added. The reaction mixture was stirred at room temperature for 16 hours. The reaction mixture was filtered and purified directly by prep. acidic HPLC to afford the title compound as a colourless solid (6.4 mg, 48% yield). 1H NMR (600 MHz, DMSO-d6) δ 11.06 (s, 1H), 8.45 (d, J=8.6 Hz, 1H), 8.26 (d, J=8.5 Hz, 1H), 7.73 (d, J=8.5 Hz, 1H), 7.49 (d, J=2.0 Hz, 1H), 6.98 (d, J=2.0 Hz, 1H), 6.59 (t, J=53.8 Hz, 1H), 4.95 (t, J=8.0 Hz, 1H), 4.55-4.36 (m, 2H), 2.00 (s, 6H), 1.28 (t, J=7.1 Hz, 3H), 0.99 (tq, J=8.4, 5.2, 4.4 Hz, 1H), 0.88 (ddt, J=13.1, 10.1, 6.5 Hz, 1H), 0.79 (td, J=9.5, 7.4 Hz, 1H), 0.53-0.44 (m, 1H), 0.39 (tdd, J=8.7, 5.5, 3.9 Hz, 1H), 0.35-0.26 (m, 2H), 0.26-0.17 (m, 4H); LCMS (ES): m/z 512.259 [M+H]⁺; RT=2.31 min.

The examples listed in the table below were all accessed using the method as described for Example 122.

| | Precursor | | | | |
|---|---|---|---|---|---|
| Ex. | Prep. | | | LCMS | Mass |
| no. | number | Structure | Name | RT | ion |
| 123 | 253 | 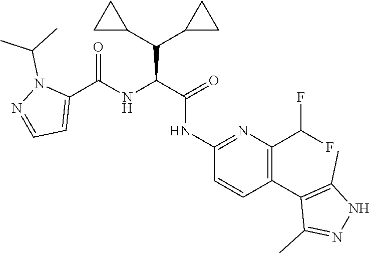 [SMILES: Cc1n[nH]c(C)c1-c1ccc(NC(=O)[C@@H](NC(=O)c2ccnn2C(C)C)C(C2CC2)C2CC2)nc1C(F)F] | N-[(1S)-1- (dicyclopropylmethyl)-2- [[6-(difluoromethyl)-5- (3,5-dimethyl-1H- pyrazol-4-yl)-2- pyridyl]amino]-2-oxo- ethyl]-2-isopropyl- pyrazole-3-carboxamide | 2.38 | 526.275 |
| 124 | 257 | 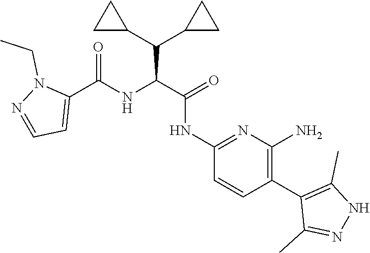 [SMILES: CCn1nccc1C(=O)N[C@H](C(=O)Nc1ccc(-c2c(C)n[nH]c2C)c(N)n1)C(C1CC1)C1CC1] | N-[(1S)-1-[[6-amino-5- (3,5-dimethyl-1H- pyrazol-4-yl)-2- pyridyl]carbamoyl]-2,2- dicyclopropyl-ethyl]-2- ethyl-pyrazole-3- carboxamide | 2.07 | 477.273 |

### Example 112: N-[(1S)-1-(dicyclopropylmethyl)-2-[[5-(3,5-dimethylisoxazol-4-yl)-6-fluoro-2-pyridyl]amino]-2-oxo-ethyl]-2-ethyl-pyrazole-3-carboxamide, and Example 113: N-[(1R)-1-(dicyclopropylmethyl)-2-[[5-(3,5-dimethylisoxazol-4-yl)-6-fluoro-2-pyridyl]amino]-2-oxo-ethyl]-2-ethyl-pyrazole-3-carboxamide

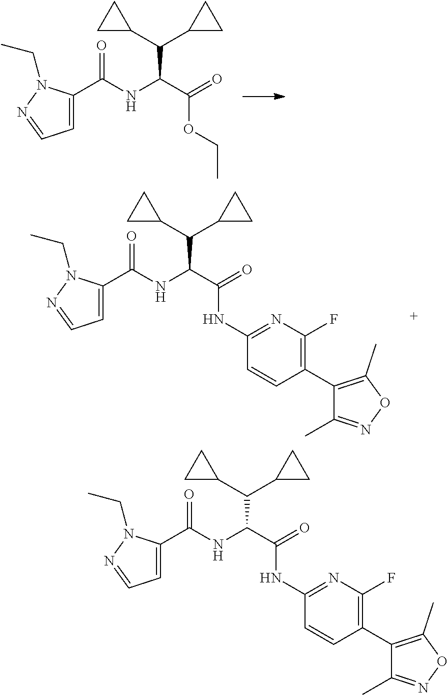

AlMe₃ (2M solution in toluene, 0.39 mL, 0.79 mmol) was added to a solution of the compound of Preparation 44 (80 mg, 0.26 mmol) and the compound of Preparation 234 (54.3 mg, 0.26 mmol) in toluene (3 mL) in a 5 mL microwave vial, under a constant nitrogen stream. The reaction mixture was stirred for 3-4 minutes, vented to release pressure and then sealed and stirred at 45° C. for 16 hours. The cooled reaction mixture was carefully quenched with H₂O (10 mL). The reaction mixture was extracted with EtOAc (2×30 mL). The combined organic extracts were dried over Na₂SO₄, filtered and concentrated in vacuo. The obtained crude compound was purified by silica column chromatography (230-400 mesh), eluting with EtOAc in heptane, to afford the title compound (45 mg, 36% yield). Chiral HPLC indicated partial racemization had occurred (69% & 30%; RT: 1.41 min & 2.72 min, Column: CHIRALPAK IC-3 (4.6*150 mm) 3 μm, Co-solvent: 0.5% DEA in Methanol, Total flow: 3 g/min, % of Co-Solvent: 35, ABPR: 1500 psi, Temperature: 30° C.) and the two compounds were separated by prep. SFC to afford the title compounds:

Example 112: N-[(1S)-1-(dicyclopropylmethyl)-2-[[5-(3,5-dimethylisoxazol-4-yl)-6-fluoro-2-pyridyl]amino]-2-oxo-ethyl]-2-ethyl-pyrazole-3-carboxamide: off-white solid (26 mg, 20% yield). 1H NMR (400 MHz, DMSO-d6): δ 11.04 (s, 1H), 8.47 (br d, J=8.1 Hz, 1H), 8.12-8.07 (m, 1H), 8.05-7.97 (m, 1H), 7.50 (d, J=2.0 Hz, 1H), 6.99 (d, J=1.9 Hz, 1H), 4.90 (br t, J=7.4 Hz, 1H), 4.52-4.42 (m, 2H), 2.35 (s, 3H), 2.16 (s, 3H), 1.28 (t, J=7.2 Hz, 3H), 1.03-0.93 (m, 1H), 0.89-0.82 (m, 1H), 0.80-0.72 (m, 1H), 0.51-0.45 (m, 1H), 0.42-0.34 (m, 1H), 0.33-0.13 (m, 6H); LCMS (ES): m/z 481.237 [M+H]⁺; RT=2.47 min; Chiral HPLC: 99.93% (RT: 1.41 min), Column: CHIRALPAK IC-3 (4.6*150 mm) 3 μm, Co-Solvent: 0.5% DEA in Methanol, Column Temperature: 30° C., Flow: 3 g/min, ABPR: 1500 psi.

Example 113: N-[(1R)-1-(dicyclopropylmethyl)-2-[[5-(3,5-dimethylisoxazol-4-yl)-6-fluoro-2-pyridyl]amino]-2-oxo-ethyl]-2-ethyl-pyrazole-3-carboxamide: off-white solid (7 mg, 5.6% yield); 1H NMR (400 MHz, DMSO-d6): δ 11.04 (s, 1H), 8.48 (br d, J=8.3 Hz, 1H), 8.12-8.06 (m, 1H), 8.05-7.98 (m, 1H), 7.50 (d, J=2.0 Hz, 1H), 7.00 (d, J=1.9 Hz, 1H), 4.90 (br t, J=7.7 Hz, 1H), 4.52-4.42 (m, 2H), 2.35 (s, 3H), 2.16 (s, 3H), 1.28 (t, J=7.2 Hz, 3H), 1.03-0.92 (m, 1H), 0.90-0.82 (m, 1H), 0.81-0.73 (m, 1H), 0.51-0.44 (m, 1H), 0.40-0.15 (m, 7H); LCMS (ES): m/z 481.237 [M+H]⁺; RT=2.47 min; Chiral HPLC: 99.90% (RT: 2.69 min), Column: CHIRALPAK IC-3 (4.6*150 mm) 3 μm, Co-Solvent: 0.5% DEA in Methanol, Column Temperature: 30° C., Flow: 3 g/min, ABPR: 1500 psi.

### Example 114: N-[(1S)-1-(dicyclopropylmethyl)-2-[[5-(3,5-dimethyltriazol-4-yl)-6-fluoro-2-pyridyl]amino]-2-oxo-ethyl]-2-ethyl-pyrazole-3-carboxamide, and

### Example 115: N-[(1R)-1-(dicyclopropylmethyl)-2-[[5-(3,5-dimethyltriazol-4-yl)-6-fluoro-2-pyridyl]amino]-2-oxo-ethyl]-2-ethyl-pyrazole-3-carboxamide

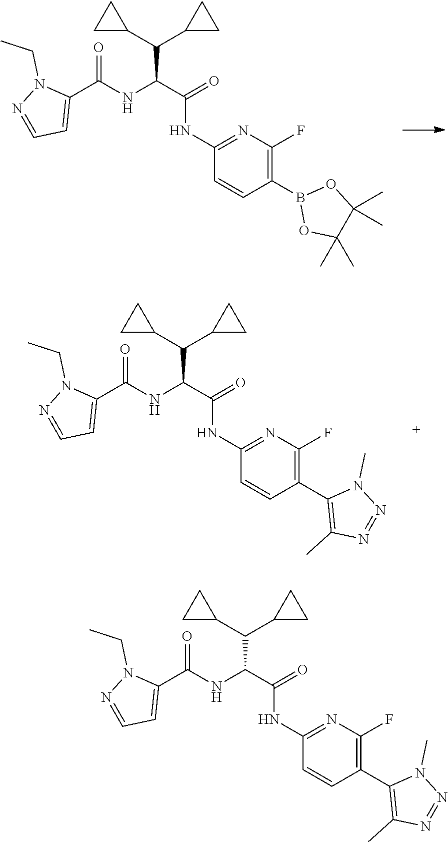

Na₂CO₃ (106 mg, 0.58 mmol) was added to a solution of the compound of Preparation 236 (75 mg, 0.14 mmol) and 5-iodo-1,4-dimethyl-1H-1,2,3-triazole (65 mg, 0.29 mmol) in toluene (5 mL) and H₂O (0.5 mL). The reaction mixture was purged with argon for 10 mins before Pd(dppf)Cl₂·DCM (18 mg, 0.021 mmol) was added and the reaction mixture was irradiated under microwave conditions at 140° C. for 40 minutes. The cooled reaction mixture was filtered through Celite, washing with EtOAc (40 mL). The filtrate was dried over Na₂SO₄, filtered and concentrated in vacuo to afford the title compound after prep. HPLC (25 mg, assume 36% yield). Chiral HPLC showed some epimerization (probably during AlMe₃ step) so the stereoisomers were separated by prep. SFC.

Example 114: N-[(1S)-1-(dicyclopropylmethyl)-2-[[5-(3,5-dimethyltriazol-4-yl)-6-fluoro-2-pyridyl]amino]-2-oxo-ethyl]-2-ethyl-pyrazole-3-carboxamide; (7 mg, colourless solid). 1H NMR (400 MHz, DMSO-d6) δ 11.35 (br s, 1H), 8.52 (d, J=8.0 Hz, 1H), 8.17-8.10 (m, 2H), 7.49 (d, J=2.0 Hz, 1H), 7.00 (d, J=2.0 Hz, 1H), 4.91 (t, J=7.8 Hz, 1H), 4.49-4.43 (m, 2H), 3.90 (s, 3H), 2.17 (s, 3H), 1.28 (t, J=7.2 Hz, 3H), 0.99-0.97 (m, 1H), 0.85-0.76 (m, 2H), 0.49-0.48 (m, 1H), 0.38-0.15 (m, 7H). LCMS (ES): m/z 481.248 [M+H]⁺; RT=2.28 min; Chiral HPLC: 99.88% (RT: 3.22 min), Column: CHIRALPAK IG-3 (4.6*150 mm) 3 μm, Co-Solvent: 0.5% DEA in Methanol, Column Temperature: 30° C., Flow: 3 g/min, ABPR: 1500 psi.

Example 115: N-[(1R)-1-(dicyclopropylmethyl)-2-[[5-(3,5-dimethyltriazol-4-yl)-6-fluoro-2-pyridyl]amino]-2-oxo-ethyl]-2-ethyl-pyrazole-3-carboxamide (1.5 mg, off-white solid); 1H NMR (400 MHz, DMSO-d6) δ 11.35 (br s, 1H), 8.52 (d, J=8.4 Hz, 1H), 8.17-8.10 (m, 2H), 7.49 (d, J=2.0 Hz, 1H), 7.00 (d, J=2.0 Hz, 1H), 4.91 (t, J=7.8 Hz, 1H), 4.49-4.43 (m, 2H), 3.90 (s, 3H), 2.17 (s, 3H), 1.28 (t, J=7.2 Hz, 3H), 0.99-0.97 (m, 1H), 0.85-0.76 (m, 3H), 0.49-0.48 (m, 1H), 0.38-0.15 (m, 6H). LCMS (ES): m/z 481.248 [M+H]⁺; RT=2.28 min; Chiral HPLC: 99.55% (RT: 2.3 min), Column: CHIRALPAK IG-3 (4.6*150 mm) 3 μm, Co-Solvent: 0.5% DEA in Methanol, Column Temperature: 30° C., Flow: 3 g/min, ABPR: 1500 psi.

### Example 170: N-[(1S)-1-(dicyclopropylmethyl)-2-[4-(3,5-dimethyl-1H-pyrazol-4-yl)anilino]-2-oxo-ethyl]-2-(1-methyl-2-methylsulfanyl-ethyl)pyrazole-3-carboxamide

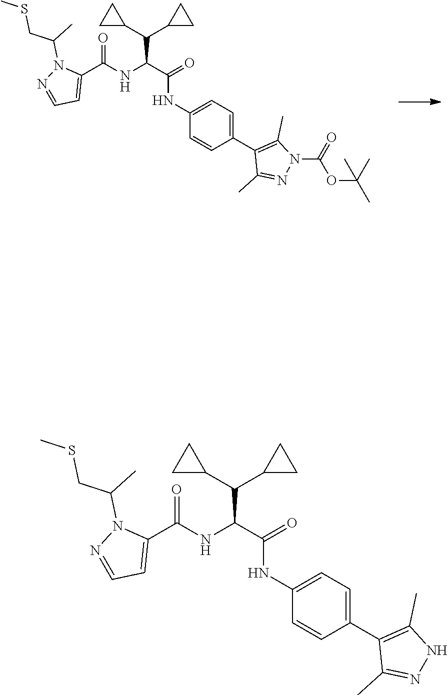

Hydrogen chloride (2.0 mL, 4M solution in 1,4-dioxane) was added to a solution of the compound of Preparation 357 (28 mg, 0.045 mmol) in MeOH (1 mL) and stirred at room temperature for 1 hour. The reaction mixture was purified directly by prep. acidic HPLC to afford the title compound as a mixture of diastereomers (21.6 mg, 91% yield). 1H NMR (400 MHz, DMSO-d6) δ 12.27 (bs, 1H), 10.18 (d, J=6.3 Hz, 1H), 8.48 (dd, J=28.5, 8.8 Hz, 1H), 7.71-7.43 (m, 3H), 7.23 (d, J=8.1 Hz, 2H), 6.97 (dd, J=30.9, 2.0 Hz, 1H), 5.55 (dq, J=36.3, 6.8 Hz, 1H), 4.83 (t, J=7.9 Hz, 1H), 2.81 (h, J=7.9, 7.3 Hz, 2H), 2.18 (s, 6H), 1.90 (d, J=36.0 Hz, 3H), 1.46 (dd, J=13.3, 6.6 Hz, 3H), 0.97-0.69 (m, 3H), 0.57-0.07 (m, 8H); LCMS (ES): m/z 512.259 [M+H]⁺; RT=2.31 min.

The examples listed in the table below were all accessed using the method as described for Example 170.

| | Precursor | | | | |
|---|---|---|---|---|---|
| Ex | Prep. | | | LCMS | Mass |
| no. | number | Structure | Name | RT | ion |
| 171 | 358 | 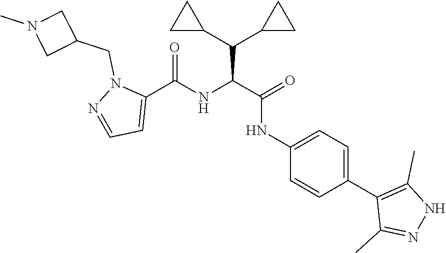 [SMILES: Cc1n[nH]c(C)c1-c1ccc(NC(=O)[C@@H](NC(=O)c2ccnn2CC2CN(C)C2)C(C2CC2)C2CC2)cc1] | N-[(1S)-1- (dicyclopropylmethyl)-2- [4-(3,5-dimethyl-1H- pyrazol-4-yl)anilino]-2- oxo-ethyl]-2-[(1- methylazetidin-3- yl)methyl]pyrazole-3- carboxamide | 1.93 | 516.309 |
| 172 | 359 | 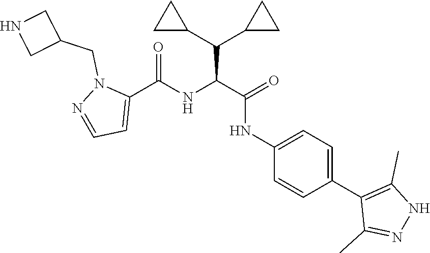 [SMILES: Cc1n[nH]c(C)c1-c1ccc(NC(=O)[C@@H](NC(=O)c2ccnn2CC2CNC2)C(C2CC2)C2CC2)cc1] | 2-(azetidin-3-ylmethyl)- N-[(1S)-1- (dicyclopropylmethyl)-2- [4-(3,5-dimethyl-1H- pyrazol-4-yl)anilino]-2- oxo-ethyl]pyrazole-3- carboxamide | 1.92 | 502.293 |

### Example 174 and Example 175: N-[(1S)-1-(dicyclopropylmethyl)-2-[4-(3,5-dimethyl-1H-pyrazol-4-yl)anilino]-2-oxo-ethyl]-2-(1-methyl-2-methylsulfinyl-ethyl)pyrazole-3-carboxamide

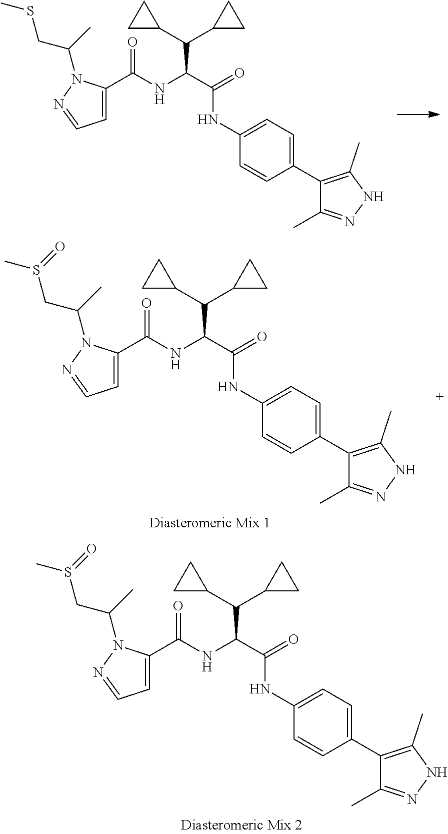 [SMILES: CSCC(C)n1nccc1C(=O)N[C@H](C(=O)Nc1ccc(-c2c(C)n[nH]c2C)cc1)C(C1CC1)C1CC1 ; Cc1n[nH]c(C)c1-c1ccc(NC(=O)[C@@H](NC(=O)c2ccnn2C(C)CS(C)=O)C(C2CC2)C2CC2)cc1 ; Cc1n[nH]c(C)c1-c1ccc(NC(=O)[C@@H](NC(=O)c2ccnn2C(C)CS(C)=O)C(C2CC2)C2CC2)cc1]

Oxone (14.5 mg, 0.236 mmol) was added to a solution of the compound of Example 170 (22.3 mg, 0.0428 mmol) in EtOH (1.1 mL) in a vial. The vial was sealed and heated at 60° C. for 16 hours. The cooled reaction mixture was filtered and purified directly on prep. HPLC to afford the 2 products as mixtures of diastereomers.

Example 174 (Diastereomeric mix 1)—8.7 mg of a colourless solid (38% yield); LCMS (ES): m/z 537.265 [M+H]⁺; RT=2.04 min.

Example 175 (Diastereomeric mix 2)—5.1 mg of a colourless solid (22% yield); LCMS (ES): m/z 537.265 [M+H]⁺; RT=2.10 min.

### Example 65: IL-8 release assay in human epithelial keratinocytes adult (HEKa)

Keratinocytes were seeded at 3500 cells/well in 384-well ViewPlates (Perkin Elmer) in Epilife medium (Thermo Fisher) containing human keratinocyte growth supplement (HKGS) without hydrocortisone and incubated in a humid incubator at 37° C., 5% CO₂, overnight. The following day growth medium was removed and 25 μl fresh Epilife medium was added. 75 nL test compound in 100% DMSO was added into each well reserved for test compounds, by the use of acoustic pipetting. The remaining wells received an equal volume of DMSO only, as vehicle control, or terfenadine in DMSO, as a positive control for any cytotoxic compounds. Subsequently, another 25 μL Epilife medium was added to each well. Finally, wells containing test compounds and wells prepared to yield maximum stimulation received 25 μL of 9 ng/mL recombinant, human embryonic kidney cell (HEK)-derived human IL-17AA+30 ng/mL human TNF-alpha, in Epilife medium. Wells prepared to define 100% inhibition of IL-17 effects received 25 μL of 30 ng/mL human TNF-alpha alone, in Epilife medium. Final concentrations were 3 ng/mL HEK-human IL-17AA+10 ng/mL human TNFalpha (maximum stimulation) and 10 ng/mL human TNFalpha alone (100% inhibition, Emax), respectively. Cells were incubated for 68-72 hours in the incubator. IL-8 released from the cells was measured by the use of a commercial homogenous time-resolved fluorescence (HTRF) assay (CisBio). 2 μL cell culture supernatant was transferred to a 384-well Proxiplate. 5 μL HTRF reagent was added and the plates were incubated, sealed in the dark, for 3-22 hours at room temperature. Time-resolved fluorescence was read at 665 vs 620 nm, with excitation at 320 nm, and IL-8 levels were calculated as percent of controls. Reduction of the amount of secreted IL-8 indicates decreased IL-17 signaling.

Concentration response curves were fitted by the use of a four-parameter logistic equation. Relative IC₅₀ and Emax were reported from curves showing acceptable fit (r²>0.9). Cytotoxicity was measured in the cell-containing Viewplates following addition of 7 μL PrestoBlue (Thermo Fisher) and incubation for 2.5-3 hours at room temperature, by measuring fluorescence at 615 nm (excitation at 535 nm). Fluorescence was directly proportional to the amount of metabolic activity. Reduction of fluorescence signal indicated cytotoxicity.

Compounds of the present invention were tested in the IL-8 release assay in human epithelial keratinocytes. The results are summarized in Table 1.

TABLE 1

| | Rel EC₅₀ IL-8 |
|---|---|
| Example No. | release assay (nM) |
| 1 | 15 |
| 2 | 17 |
| 3 | 19 |
| 4 | 130 |
| 5 | 130 |
| 6 | 150 |
| 7 | 230 |
| 8 | 250 |
| 9 | 380 |
| 10 | 330 |
| 11 | 290 |
| 12 | 320 |
| 13 | 540 |
| 14 | 600 |
| 15 | >10000 |
| 16 | 150 |
| 17 | 110 |
| 18 | 460 |
| 19 | 110 |
| 20 | 25 |
| 21 | 28 |
| 22 | 66 |
| 23 | 130 |
| 24 | 5.4 |
| 25 | 9.1 |
| 26 | 31 |
| 27 | 6.6 |
| 28 | 3.3 |
| 29 | 78 |
| 30 | 90 |
| 31 | 75 |
| 32 | 49 |
| 33 | 52 |
| 34 | 140 |
| 35 | 8.7 |
| 36 | 69 |
| 37 | 25 |
| 38 | 16 |
| 39 | 87 |
| 40 | 34 |
| 41 | 44 |
| 42 | 20 |
| 43 | 340 |
| 44 | >10000 |
| 45 | 23 |
| 46 | 1600 |
| 47 | 180 |
| 48 | 2600 |
| 49 | 280 |
| 50 | 2500 |
| 51 | 660 |
| 52 | 790 |
| 53 | 1100 |
| 54 | 1400 |
| 55 | 1600 |
| 56 | 3600 |
| 57 | 160 |
| 58 | 120 |
| 59 | 200 |
| 60 | 290 |
| 61 | 160 |
| 62 | 5000 |
| 63 | 74 |
| 64 | 820 |
| 66 | 170 |
| 67 | 77 |
| 68 | 34 |
| 69 | 120 |
| 70 | 54 |
| 71 | 33 |
| 72 | 17 |
| 73 | 16 |
| 74 | 140 |
| 75 | 76 |
| 76 | 7.4 |
| 77 | 590 |
| 78 | 76 |
| 79 | 48 |
| 80 | 9.5 |
| 81 | 17 |
| 82 | 74 |
| 83 | 46 |
| 84 | 8.1 |
| 85 | 83 |
| 86 | 21 |
| 87 | 77 |
| 88 | 35 |
| 89 | 46 |
| 90 | 29 |
| 91 | 73 |
| 92 | 13 |
| 93 | 39 |
| 94 | 30 |
| 95 | 1200 |
| 96 | 66 |
| 97 | 13 |
| 98 | 6.3 |
| 99 | 21 |
| 100 | 25 |
| 101 | 6.2 |
| 102 | 690 |
| 103 | 350 |
| 104 | 91 |
| 105 | 640 |
| 106 | 150 |
| 107 | 46 |
| 108 | 14 |
| 109 | 26 |
| 110 | 29 |
| 111 | 19 |
| 112 | 59 |
| 113 | 1300 |
| 114 | 200 |
| 115 | 8000 |
| 116 | 120 |
| 117 | 49 |
| 118 | 58 |
| 119 | 36 |
| 120 | 170 |
| 121 | 160 |
| 122 | 780 |
| 123 | 330 |
| 124 | 820 |
| 125 | 13 |
| 126 | 350 |
| 127 | 170 |
| 128 | 900 |
| 129 | 380 |
| 130 | 63 |
| 131 | 12 |
| 132 | 17 |
| 133 | 37 |
| 134 | 18 |
| 135 | 12 |
| 136 | 29 |
| 137 | 3.6 |
| 138 | 3.9 |
| 139 | 720 |
| 140 | 45 |
| 141 | 58 |
| 142 | 32 |
| 143 | 33 |
| 144 | 19 |
| 145 | 22 |
| 146 | 96 |
| 147 | 38 |
| 148 | 56 |
| 149 | 28 |
| 150 | 27 |
| 151 | 15 |
| 152 | 25 |
| 153 | 25 |
| 154 | 61 |
| 155 | 41 |
| 156 | 25 |
| 157 | 19 |
| 158 | 54 |
| 159 | 33 |
| 160 | 20 |
| 161 | 30 |
| 162 | 170 |
| 163 | 130 |
| 164 | 110 |
| 165 | 130 |
| 166 | 110 |
| 167 | 76 |
| 168 | Not tested |
| 169 | Not tested |
| 170 | 12 |
| 171 | 460 |
| 172 | 360 |
| 173 | 58 |
| 174 | 32 |
| 175 | 120 |
| 176 | 24 |
| 177 | 180 |
| 178 | 100 |
| 179 | 6.4 |
| 180 | 58 |
| 181 | 130 |
| 182 | 31 |
| 183 | 150 |
| 184 | 150 |
| 185 | 12 |
| 186 | 95 |
| 187 | 110 |
| 188 | 3.6 |
| 189 | 32 |
| 190 | 740 |

### EMBODIMENTS

Embodiment 1. A compound according to formula (I)

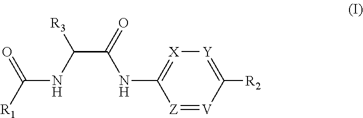

wherein

R₁ is selected from the group consisting of (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, 4-6-membered heterocycloalkyl and —NR_{c}R_{d}, wherein said (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, and 4-6-membered heterocycloalkyl is optionally substituted with one or more substituents independently selected from Rₐ;

Rₐ represents deuterium, halogen, hydroxy, —NR_{c}R_{d}, (C₁-C₆)alkyl, (C₁-C₆)alkylcarbonyl, (C₃-C₇)cycloalkyl, phenyl, 5- or 6-membered heteroaryl or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from the group consisting of 5- or 6-membered heteroaryl, wherein said 5- or 6-membered heteroaryl is optionally substituted with one or more substituents independently selected from R_{b}, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;

R_{b} represents deuterium, halogen, cyano, hydroxy, —NR_{c}R_{d}, (C₁-C₆)alkyl, (C₁-C₆)alkoxy, (C₁-C₆)alkyl-CO—O—(CH₂)n- or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from the group consisting of —CHR₅R₆, (C₃-C₁₀)cycloalkyl and G, wherein said (C₃-C₁₀)cycloalkyl and G are optionally substituted with one or more substituents independently selected from deuterium, halogen, cyano, (C₁-C₄)alkyl and halo(C₁-C₄)alkyl;

G is

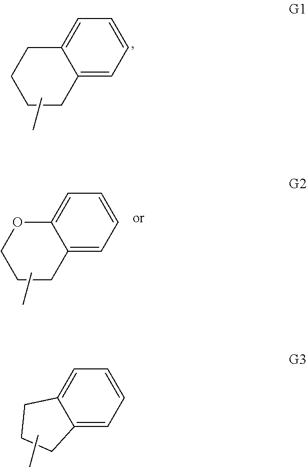

R₅ and R₆ each independently represent hydrogen, phenyl, (C₁-C₆)alkyl, (C₃-C₇)cycloalkyl or (C₃-C₇)cycloalkyl(C₁-C₆)alkyl, wherein said phenyl, (C₁-C₆)alkyl, (C₃-C₇)cycloalkyl, or (C₃-C₇)cycloalkyl(C₁-C₆)alkyl is optionally substituted with one or more substituents independently selected from halogen, cyano and (C₁-C₄)alkyl; with the proviso that at least one of R₅ and R₆ is different from hydrogen;

X, Y, Z and V are each independently selected from N, CH and C(R₄); provided that at least one of X, Y, Z and V is N;

R₄ is selected from (C₁-C₆)alkyl, (C₁-C₆)alkoxy and halogen, wherein said (C₁-C₆)alkyl and (C₁-C₆)alkoxy may optionally be substituted with one or more substituents independently selected from halogen;

provided that when R₃ is (C₁-C₄)alkyl, cyclopentyl, cyclohexylmethyl, benzyl or substituted benzyl, then R₁ is selected from the group consisting of pyrazolyl, imidazolyl, thiazolyl, isoxazolyl and triazolyl, wherein the pyrazolyl, imidazolyl, thiazolyl, isoxazolyl, or triazolyl is optionally substituted with one or more substituents independently selected from Rₐ;

or pharmaceutically acceptable salts, hydrates and solvates thereof.

Embodiment 2. The compound according to embodiment 1 having the formula (Ia)

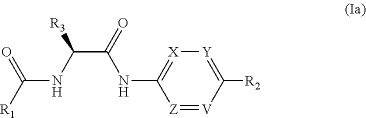

wherein R₁, R₂, R₃, X, Y, Z, and V are as defined in embodiment 1; or pharmaceutically acceptable salts, hydrates and solvates thereof.

Embodiment 3. The compound according to embodiment 1 having the formula (Ib)

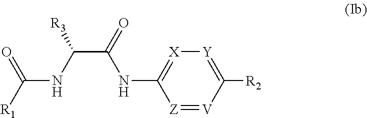

wherein R₁, R₂, R₃, X, Y, Z, and V are as defined in embodiment 1; or pharmaceutically acceptable salts, hydrates and solvates thereof.

Embodiment 4. A compound according to any one of embodiments 1-3, wherein R₃ is selected from —CHR₅R₆, and wherein R₅ and R₆ each independently represent hydrogen, phenyl, cyclopropyl, cyclobutyl, cyclopentyl, cyclohexyl, cyclopropylmethyl, cyclobutylmethyl, methyl or ethyl, wherein said phenyl, cyclopropyl, cyclobutyl, cyclopentyl, cyclohexyl, cyclopropylmethyl, cyclobutylmethyl, methyl or ethyl, is optionally substituted with one or more substituents independently selected from halogen, cyano, and (C₁-C₄)alkyl; with the proviso that at least one of R₅ and R₆ is different from hydrogen.

Embodiment 5. The compound according to any one of embodiments 1-3, wherein R₃ is selected from —CHR₅R₆, and wherein R₅ and R₆ each independently represent (C₃-C₇)cycloalkyl, wherein said (C₃-C₇)cycloalkyl is optionally substituted with one or more substituents independently selected from halogen, cyano and (C₁-C₄)alkyl.

Embodiment 6. The compound according to any one of embodiments 1-5, wherein R₃ is dicyclopropylmethyl.

Embodiment 7. The compound according to any one of embodiments 1-3, wherein R₃ is selected from cyclohexyl, cycloheptyl, cyclooctanyl, adamantyl, spiro[2.3]hexanyl, bicyclo[3,1,0]hexanyl, bicyclo[4,1,0]heptanyl and bicyclo[2,2,2]octanyl or spiro[2.5]octanyl, wherein said cyclohexyl, cycloheptyl, cyclooctanyl, adamantyl, spiro[2.3]hexanyl, bicyclo[3,1,0]hexanyl, bicyclo[4,1,0]heptanyl and bicyclo[2,2,2]octanyl or spiro[2.5]octanyl is optionally substituted with one or more substituents independently selected from deuterium, halogen, cyano, (C₁-C₄)alkyl and halo(C₁-C₄)alkyl.

Embodiment 8. A compound according to any one of embodiments 1-3 and 7, wherein R₃ is cyclohexyl optionally substituted with (C₁-C₄)alkyl.

Embodiment 9. The compound according to any one of the embodiments 1-3, wherein R₃ is selected from G, wherein G represents

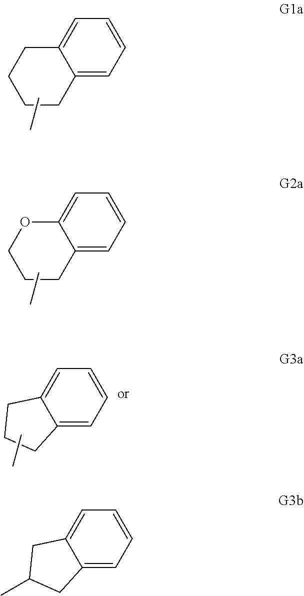

wherein said G is optionally substituted with one or more substituents independently selected from deuterium, halogen, cyano, (C₁-C₄)alkyl and halo(C₁-C₄)alkyl.

Embodiment 10. The compound according to any one of the embodiments 1-9, wherein R₁ is selected from (C₁-C₆)alkyl, (C₁-C₆)alkoxy, (C₃-C₇)cycloalkyl, 5-membered heteroaryl, 9-membered bicyclic heteroaryl and 4-6-membered heterocycloalkyl, wherein said (C₁-C₆)alkyl, (C₁-C₆)alkoxy, (C₃-C₇)cycloalkyl, 5-membered heteroaryl, 9-membered bicyclic heteroaryl and 4-6-membered heterocycloalkyl is optionally substituted with one or more substituents independently selected from Rₐ.

Embodiment 11. The compound according to any one of embodiments 1-10 wherein R₁ is selected from pyrazolyl, imidazolyl, thiazolyl, isoxazolyl and triazolyl, wherein said pyrazolyl, imidazolyl, thiazolyl, isoxazolyl and triazolyl is optionally substituted with one or more substituents independently selected from Rₐ.

Embodiment 12. The compound according to any one of embodiments 1-11 wherein R₁ is selected from pyrazolyl, imidazolyl, thiazolyl, isoxazolyl and triazolyl, wherein said pyrazolyl, imidazolyl, thiazolyl, isoxazolyl and triazolyl is optionally substituted with one or more substituents independently selected from (C₁-C₆)alkyl, wherein said (C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d}.

Embodiment 13. The compound according to any one of embodiments 1-12 wherein R₁ is selected from pyrazolyl, wherein said pyrazolyl is optionally substituted with one or more substituents independently selected from (C₁-C₆)alkyl, wherein said (C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d}.

Embodiment 14. The compound according to any one of embodiments 1-13, wherein R₁ is selected from 2-((C₁-C₆)alkyl)-pyrazol-3-yl, wherein said (C₁-C₆)alkyl in position 2 on the pyrazol-3-yl is optionally substituted with one or more substituents selected from halogen, hydroxy, (C₁-C₄)alkoxy, (C₁-C₄)alkyl-S—, (C₁-C₄)alkyl-SO— and (C₁-C₄)alkyl-SO₂—.

Embodiment 15. The compound according to any one of embodiments 1-14 wherein R₁ is 2-((C₁-C₆)alkyl)-pyrazol-3-yl.

Embodiment 16. The compound according to any one of embodiments 1-15 wherein R₁ is 2-(isopropyl)-pyrazol-3-yl or 2-(ethyl)-pyrazol-3-yl.

Embodiment 17. The compound according to any one of embodiments 1-16 wherein R₂ is selected from pyrazolyl and imidazolyl, wherein said pyrazolyl or imidazolyl is optionally substituted with one or more substituents independently selected from R_{b}.

Embodiment 18. The compound according to any one of embodiments 1-17, wherein R₂ is pyrazol-4-yl or imidazole-4-yl, wherein said pyrazol-4-yl or imidazol-4-yl is substituted with one or more substituents independently selected from (C₁-C₆)alkyl.

Embodiment 19. The compound according to any one of embodiments 1-18, wherein R₂ is 3,5-dimethyl-pyrazol-4-yl.

Embodiment 20. The compound according to any one of embodiments 1-16 wherein R₂ is selected from pyrazol-4-yl or imidazol-4-yl, wherein said pyrazol-4-yl or imidazol-4-yl contain a nitrogen ring atom substituted with a substituent selected from -L-PO(OH)₂ and the other ring atoms of said pyrazol-4-yl or imidazole-4-yl is substituted with one or more substituents independently selected from (C₁-C₆)alkyl.

Embodiment 21. The compound according to any one of embodiments 1-20, wherein X is N and Y, Z and V are independently selected from CH and C(R₄).

Embodiment 22. The compound according to any one of embodiments 1-20, wherein Y is N and X, Z and V are independently selected from CH and C(R₄).

Embodiment 23. The compound according to any one of embodiments 1-20, wherein X and Y are N and V and Z are independently selected from CH and C(R₄).

Embodiment 24. The compound according to any one of embodiments 1-20, wherein Y and Z are N and X and V are independently selected from CH and C(R₄).

Embodiment 25. The compound according to any one of embodiments 1-20, wherein X and Z are N and Y and V are independently selected from CH and C(R₄).

Embodiment 26. The compound according to any one of embodiments 1-20, wherein Y and V are N and Z and X are independently selected from CH and C(R₄).

Embodiment 27. The compound according to any one of embodiments 1-20, wherein X, Y and Z are N and V is selected from CH and C(R₄).

Embodiment 28. The compound according to any one of embodiments 1-20, wherein X, Y and V are N and Z is selected from CH and C(R₄).

Embodiment 29. The compound according to any one of embodiments 1-20, wherein X is N, Y is C(R₄) and V and Z are CH.

Embodiment 30. The compound according to embodiment 29, wherein X is N, Y is C(R₄) and V and Z are CH and R₄ is halogen, such as fluoro.

Embodiment 31. The compound according to any one of embodiments 1-3, wherein

R₁ is selected from pyrazolyl, imidazolyl, thiazolyl, isoxazolyl and triazolyl, wherein said pyrazolyl, imidazolyl, thiazolyl, isoxazolyl and triazolyl is optionally substituted with a substituent independently selected from Rₐ.

Rₐ represents deuterium, halogen, hydroxy, —NR_{c}R_{d}, (C₁-C₆)alkyl, (C₁-C₆)alkylcarbonyl, (C₃-C₇)cycloalkyl, phenyl, 5- or 6-membered heteroaryl or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from pyrazolyl and imidazolyl, wherein said pyrazolyl or imidazolyl is optionally substituted with one or more substituents independently selected from R_{b} and wherein when said 5 membered heteroaryl contains nitrogen as a ring atom said nitrogen may optionally be substituted with -L-PO(OH)₂;

R_{b} represents deuterium, halogen, cyano, hydroxy, —NR_{c}R_{d}, (C₁-C₆)alkyl, (C₁-C₆)alkoxy, (C₁-C₆)alkyl-CO—O—(CH₂)n- or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from cyclohexyl, cycloheptyl, cyclooctanyl, adamantyl, spiro[2.3]hexanyl, bicyclo[3,1,0]hexanyl, bicyclo[4,1,0]heptanyl and bicyclo[2,2,2]octanyl or spiro[2.5]octanyl, wherein said cyclohexyl, cycloheptyl, cyclooctanyl, adamantyl, spiro[2.3]hexanyl, bicyclo[3,1,0]hexanyl, bicyclo[4,1,0]heptanyl and bicyclo[2,2,2]octanyl or spiro[2.5]octanyl is optionally substituted with one or more substituents independently selected from deuterium, halogen, cyano, (C₁-C₄)alkyl and halo(C₁-C₄)alkyl;

X is N, Y is C(R₄) and V and Z are CH;

R₄ is selected from (C₁-C₆)alkyl, (C₁-C₆)alkoxy and halogen; wherein said (C₁-C₆)alkyl and (C₁-C₆)alkoxy may optionally be substituted with one or more substituents independently selected from halogen;

or pharmaceutically acceptable salts, hydrates and solvates thereof.

Embodiment 32. The compound according to embodiment 31, wherein R₁ is pyrazol-3-yl substituted with one or more (C₁-C₄)alkyl, wherein said one or more (C₁-C₄)alkyl is optionally substituted with a substituent selected from (C₁-C₄)alkoxy, (C₁-C₄)alkyl-S—, (C₁-C₄)alkyl-SO—, and (C₁-C₄)alkyl-SO₂—, R₂ is pyrazol-4-yl substituted with one or more (C₁-C₄)alkyl, R₃ is cyclopentyl, cyclohexyl or cycloheptanyl optionally substituted with (C₁-C₄)alkyl and X is N, Y is C(R₄), wherein R₄ is halogen and V and Z are CH.

Embodiment 33. The compound according to embodiment 32 wherein R₁ is 2-(C₁-C₃)alkyl-pyrazol-3-yl, R₂ is 3,5-di(C₁-C₂)alkyl-pyrazol-4-yl, R₃ is cyclohexyl substituted with (C₁-C₄)alkyl, and X is N, Y is C(R₄), wherein R₄ is fluoro and V and Z are CH.

Embodiment 34. The compound according to any one of embodiments 1-3, wherein

R₁ is selected from (C₃-C₇)cycloalkyl and (C₃-C₇)cycloalkoxy, wherein said (C₃-C₇)cycloalkyl and (C₃-C₇)cycloalkoxy, is optionally substituted with a substituent independently selected from Rₐ.

Rₐ represents deuterium, halogen, hydroxy, —NR_{c}R_{d}, (C₁-C₆)alkyl, (C₁-C₆)alkylcarbonyl, (C₃-C₇)cycloalkyl, phenyl, 5- or 6-membered heteroaryl or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from pyrazolyl and imidazolyl, wherein said pyrazolyl or imidazolyl is optionally substituted with one or more substituents independently selected from R_{b} and wherein when said 5 membered heteroaryl contains nitrogen as a ring atom said nitrogen may optionally be substituted with -L-PO(OH)₂;

R_{b} represents deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from cyclohexyl, cycloheptyl, cyclooctanyl, adamantyl, spiro[2.3]hexanyl, bicyclo[3,1,0]hexanyl, bicyclo[4,1,0]heptanyl and bicyclo[2,2,2]octanyl or spiro[2.5]octanyl, wherein said cyclohexyl, cycloheptyl, cyclooctanyl, adamantyl, spiro[2.3]hexanyl, bicyclo[3,1,0]hexanyl, bicyclo[4,1,0]heptanyl and bicyclo[2,2,2]octanyl or spiro[2.5]octanyl is optionally substituted with one or more substituents independently selected from deuterium, halogen, cyano, (C₁-C₄)alkyl and halo(C₁-C₄)alkyl;

X is N, Y is C(R₄) and V and Z are CH;

R₄ is selected from (C₁-C₆)alkyl, (C₁-C₆)alkoxy and halogen; wherein said (C₁-C₆)alkyl and (C₁-C₆)alkoxy may optionally be substituted with one or more substituents independently selected from halogen;

or pharmaceutically acceptable salts, hydrates and solvates thereof.

Embodiment 35. The compound according to embodiment 34, wherein R₁ is (C₃-C₇)cycloalkyl, wherein said (C₃-C₇)cycloalkyl is optionally substituted by one or more substituents selected from halogen, R₂ is pyrazol-4-yl substituted with one or more (C₁-C₄)alkyl, R₃ is cyclopentyl, cyclohexyl or cycloheptanyl optionally substituted with (C₁-C₄)alkyl and X is N, Y is C(R₄), wherein R₄ is halogen and V and Z are CH.

Embodiment 36. The compound according to embodiment 35 wherein R₁ is 1-fluoro-cyclopropyl, R₂ is 3,5-di(C₁-C₂)alkyl-pyrazol-4-yl, R₃ is cyclohexyl substituted with (C₁-C₄)alkyl, and X is N, Y is C(R₄), wherein R₄ is fluoro and V and Z is CH.

Embodiment 37. The compound according to any one of claims 1-2, wherein

R₁ is selected from (C₃-C₇)cycloalkyl and (C₃-C₇)cycloalkoxy, wherein said (C₃-C₇)cycloalkyl and (C₃-C₇)cycloalkoxy, is optionally substituted with a substituent independently selected from Rₐ;

Rₐ represents deuterium, halogen, hydroxy, —NR_{c}R_{d}, (C₁-C₆)alkyl, (C₁-C₆)alkylcarbonyl, (C₃-C₇)cycloalkyl, phenyl, 5- or 6-membered heteroaryl or 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR_{c}R_{d};

R₂ is selected from pyrazolyl and imidazolyl, wherein said pyrazolyl or imidazolyl is optionally substituted with one or more substituents independently selected from R_{b} and wherein when said 5 membered heteroaryl contains nitrogen as a ring atom said nitrogen may optionally be substituted with -L-PO(OH)₂;

R_{b} represents deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ— or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR_{c}R_{d} and (C₁-C₄)alkoxy;

R_{c} and R_{d} each independently are selected from the group consisting of hydrogen and (C₁-C₆)alkyl, or R_{c} and R_{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;

L is selected from the group consisting of a bond or —CHR_{g}O—,

R_{g} is selected from hydrogen and (C₁-C₆)alkyl;

R₃ is selected from —CHR₅R₆, and wherein R₅ and R₆ each independently represent (C₃-C₇)cycloalkyl, wherein said (C₃-C₇)cycloalkyl is optionally substituted with one or more substituents independently selected from halogen, cyano, and (C₁-C₄)alkyl;

X is N, Y is C(R₄) and V and Z are CH;

R₄ is selected from (C₁-C₆)alkyl, (C₁-C₆)alkoxy and halogen; wherein said (C₁-C₆)alkyl and (C₁-C₆)alkoxy may optionally be substituted with one or more substituents independently selected from halogen;

or pharmaceutically acceptable salts, solvates and hydrates thereof.

Embodiment 38. A compound according to embodiment 37, wherein R₁ is (C₃-C₇)cycloalkyl, wherein said (C₃-C₇)cycloalkyl is optionally substituted by one or more substituents selected from halogen, R₃ is —CHR₅R₆, and wherein R₅ and R₆ each independently represent (C₃-C₄)cycloalkyl, and X is N, Y is C(R₄), wherein R₄ is halogen and V and Z are CH.

Embodiment 39. A compound according to claim 38, wherein R₁ is 1-fluoro-cyclopropyl, R₂ is 3,5-di(C₁-C₂)alkyl-pyrazol-4-yl, R₃ is —CHR₅R₆, and wherein R₅ and R₆ each independently represent (C₃-C₄)cycloalkyl, and X is N, Y is C(R₄), wherein R₄ is fluoro and V and Z are CH.

Embodiment 40. The compound according to any one of the embodiments above wherein R₃ is selected from —CHR₅R₆, and wherein R₅ and R₆ each independently represent (C₃-C₄)cycloalkylmethyl, wherein said (C₃-C₄)cycloalkylmethyl is optionally substituted with one or more substituents independently selected from halogen, cyano, and (C₁-C₄)alkyl.

Embodiment 41. The compound according to any of the embodiments above wherein R₃ is selected from —CHR₅R₆, and wherein R₅ and R₆ each independently is cyclopropylmethyl.

Embodiment 42. A compound according to any embodiment or claim herein wherein R₁ is not tertbutyloxy or benzyloxy.

Embodiment 43. A compound according to any embodiment or claim herein wherein R₁ is isoxazolyl, wherein said isoxazolyl is optionally substituted with one or more substituents independently selected from (C₁-C₆)alkyl and (C₃-C₇)cycloalkyl-(C₁-C₆)alkyl wherein said one or more (C₁-C₆)alkyl and (C₃-C₄)cycloalkyl-(C₁-C₂)alkyl is optionally substituted with one or more substituents independently selected from halogen, hydroxy, (C₁-C₄)alkoxy, (C₁-C₄)alkyl-S—, (C₁-C₄)alkyl-SO—, and (C₁-C₄)alkyl-SO₂—

Embodiment 44. A compound according to any embodiment or claim herein wherein R₁ is isoxazol-4-yl substituted with one substituent selected from (C₁-C₄)alkyl and (C₃-C₄)cycloalkyl-(C₁-C₂)alkyl wherein said (C₁-C₄)alkyl and (C₃-C₄)cycloalkyl-(C₁-C₂)alkyl is optionally substituted with a substituent selected from halogen, hydroxy, (C₁-C₄)alkoxy, (C₁-C₄)alkyl-S—, (C₁-C₄)alkyl-SO— and (C₁-C₄)alkyl-SO₂—, R₂ is pyrazol-4-yl substituted with one or more (C₁-C₄)alkyl or deutorated (C₁-C₄)alkyl, R₃ is —CHR₅R₆, wherein R₅ and R₆ each independently represent (C₃-C₇)cycloalkyl.

1. A compound according to formula (I) 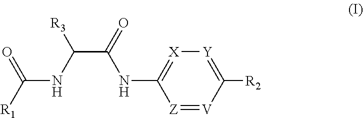
wherein
X, Y, Z and V are each independently selected from N, CH and C(R₄); R₄ is independently selected from (C₁-C₆)alkyl, (C₁-C₆)alkoxy, hydroxy, NH₂, and halogen, wherein said (C₁-C₆)alkyl and (C₁-C₆)alkoxy may optionally be substituted with one or more substituents independently selected from a halogen;
R₁ is selected from the group consisting of (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, 4-6-membered heterocycloalkyl, and —NR_{c}R_{d}, wherein said (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, and 4-6-membered heterocycloalkyl is optionally substituted with one or more substituents independently selected from Rₐ;
Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-SO₂—, and —NR_{c}R_{d};
R₂ is selected from the group consisting of 5- or 6-membered heteroaryl, wherein said 5- or 6-membered heteroaryl is optionally substituted with one or more substituents independently selected from R_{b}, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;
R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
R_{c} and R_{d} each independently are selected from the group consisting of hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano, and hydroxy;
L is selected from the group consisting of a bond or —CHR_{g}O—,
R_{g} is selected from hydrogen and (C₁-C₆)alkyl;
R₃ is selected from the group consisting of —CHR₅R₆, (C₃-C₁₀)cycloalkyl, and G, wherein said (C₃-C₁₀)cycloalkyl and G are optionally substituted with one or more substituents independently selected from deuterium, halogen, cyano, (C₁-C₄)alkyl, and halo(C₁-C₄)alkyl;
G is 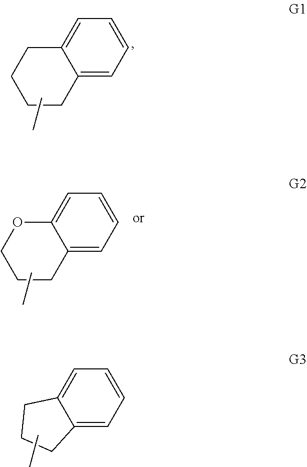
R₅ and R₆ each independently represent hydrogen, phenyl, (C₁-C₆)alkyl, (C₃-C₇)cycloalkyl, and (C₃-C₇)cycloalkyl(C₁-C₆)alkyl wherein said phenyl, (C₁-C₆)alkyl, (C₃-C₇)cycloalkyl, and (C₃-C₇)cycloalkyl(C₁-C₆)alkyl is optionally substituted with one or more substituents independently selected from halogen, cyano, and (C₁-C₄)alkyl; with the proviso that at least one of R₅ and R₆ is different from hydrogen;
provided that when R₃ is (C₁-C₄)alkyl, cyclopentyl, cyclohexylmethyl, benzyl, or substituted benzyl, then R₁ is selected from the group consisting of pyrazolyl, imidazolyl, thiazolyl, isoxazolyl, oxadiazolyl, and triazolyl, wherein the pyrazolyl, imidazolyl, thiazolyl, isoxazolyl, oxadiazolyl, or triazolyl is optionally substituted with one or more substituents independently selected from Rₐ; and
provided that when all of X, Y, Z, and V are C or C(R₄) then:
Rₐ is (C₁-C₆)alkyl substituted with one or more substituents independently selected from (C₁-C₄)alkyl-S— or (C₁-C₄)alkyl-SO—; or
Rₐ is —NR_{c}R_{d}, wherein R_{c} and R_{d} together form azetidinyl or azetidinyl optionally substituted with one or more substituents independently selected from halogen, cyano, and hydroxy; or
Rₐ is 4-6-membered heterocycloalkyl-(C₁-C₆)alkyl wherein said 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-SO₂—, and —NR_{c}R_{d}; or
Rₐ is (C₃-C₇)cycloalkyl-(C₁-C₆)alkyl substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-SO₂—, and —NR_{c}R_{d}; or
R₃ is —CHR₅R₆, wherein at least one of R₅ and R₆ is (C₃-C₇)cycloalkyl(C₁-C₆)alkyl wherein said (C₃-C₇)cycloalkyl(C₁-C₆)alkyl is optionally substituted with one or more substituents independently selected from halogen, cyano, and (C₁-C₄)alkyl,
or a pharmaceutically acceptable salt, hydrate, or solvate thereof.

2. The compound according to claim 1 having the formula (Ia) 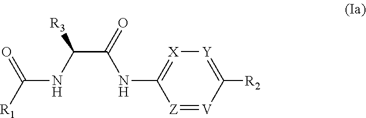
or a pharmaceutically acceptable salt, hydrate, or solvate thereof.

3. The compound according to claim 1 having the formula (Ib) 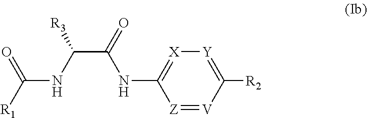
or a pharmaceutically acceptable salt, hydrate, or solvate thereof.

4. The compound according to claim 1 wherein X, Y, Z, and V are each independently selected from N, CH, and C(R₄); provided that at least one of X, Y, Z, and V is N.

5. (canceled)

6. The compound according to claim 1 wherein
R₁ is selected from pyrazolyl, imidazolyl, thiazolyl, isoxazolyl, oxadiazolyl, and triazolyl, wherein said pyrazolyl, imidazolyl, thiazolyl, isoxazolyl, oxodiazolyl, and triazolyl is optionally substituted with one or more substituents independently selected from Rₐ;
Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-SO₂—, and —NR_{c}R_{d};
R₂ is a 5- or 6-membered heteroaryl, wherein said 5- or 6-membered heteroaryl is optionally substituted with one or more substituents independently selected from R_{b}, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;
R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
R_{c} and R_{d} each independently are selected from the group consisting of hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano, and hydroxy;
L is a bond or —CHR_{g}O—,
R_{g} is hydrogen or (C₁-C₆)alkyl;
R₃ is —CHR₅R₆, and wherein R₅ and R₆ each independently represent (C₃-C₇)cycloalkyl, or (C₃-C₇)cycloalkyl(C₁-C₆)alkyl, wherein said (C₃-C₇)cycloalkyl or (C₃-C₇)cycloalkyl(C₁-C₆)alkyl is optionally substituted with one or more substituents independently selected from halogen, cyano, and (C₁-C₄)alkyl;
X, Y, Z, and V are each independently selected from N, CH, and C(R₄); provided that at least one of X, Y, Z, and V is N;
R₄ is independently selected from (C₁-C₆)alkyl, (C₁-C₆)alkoxy, hydroxy, NH₂, and halogen; wherein said (C₁-C₆)alkyl and (C₁-C₆)alkoxy may optionally be substituted with one or more substituents independently selected from halogen;
or a pharmaceutically acceptable salt, hydrate, or solvate thereof.

7. The compound according to of claim 1, wherein
R₁ is selected from pyrazolyl, imidazolyl, thiazolyl, isoxazolyl, oxadiazolyl, and triazolyl, wherein said pyrazolyl, imidazolyl, thiazolyl, isoxazolyl, oxadiazolyl, and triazolyl is optionally substituted with one or more substituents independently selected from Rₐ,
Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-SO₂—, and —NR_{c}R_{d};
R₂ is a 5- or 6-membered heteroaryl, wherein said 5- or 6-membered heteroaryl is optionally substituted with one or more substituents independently selected from R_{b}, wherein said 5- or 6-membered heteroaryl may optionally contain —CO— as a ring member and wherein when said 5 membered heteroaryl contains nitrogen as a ring atom said nitrogen may optionally be substituted with -L-PO(OH)₂;
R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
R_{c} and R_{d} each independently are selected from the group consisting of hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano, and hydroxy;
L is a bond or —CHR_{g}O—,
R_{g} is hydrogen or (C₁-C₆)alkyl;
R₃ is selected from cyclohexyl, cycloheptyl, cyclooctanyl, adamantyl, spiro[2.3]hexanyl, bicyclo[3,1,0]hexanyl, bicyclo[4,1,0]heptanyl, bicyclo[2,2,2]octanyl, or spiro[2.5]octanyl, wherein said cyclohexyl, cycloheptyl, cyclooctanyl, adamantyl, spiro[2.3]hexanyl, bicyclo[3,1,0]hexanyl, bicyclo[4,1,0]heptanyl, bicyclo[2,2,2]octanyl, or spiro[2.5]octanyl is optionally substituted with one or more substituents independently selected from deuterium, halogen, cyano, (C₁-C₄)alkyl, and halo(C₁-C₄)alkyl;
X, Y, Z, and V are each independently selected from N, CH, and C(R₄); provided that at least one of X, Y, Z, and V is N;
R₄ is independently selected from (C₁-C₆)alkyl, (C₁-C₆)alkoxy, hydroxy, NH₂, and halogen; wherein said (C₁-C₆)alkyl and (C₁-C₆)alkoxy may optionally be substituted with one or more substituents independently selected from halogen;
or a pharmaceutically acceptable salt, hydrate, or solvate thereof.

8. (canceled)

9. The compound according to claim 1 wherein R₂ is selected from pyrazolyl and imidazolyl, wherein said pyrazolyl or imidazolyl is optionally substituted with one or more substituents independently selected from R_{b}.

10. The compound according to claim 9, wherein R₂ is pyrazol-4-yl or imidazol-4-yl, wherein said pyrazol-4-yl or imidazol-4-yl is substituted with one or more substituents independently selected from (C₁-C₆)alkyl and deuterated (C₁-C₆)alkyl.

11. (canceled)

12. The compound according to claim 1, wherein R₁ is pyrazolyl or triazolyl wherein said pyrazolyl or triazolyl is optionally substituted with one or more substituents independently selected from (C₁-C₆)alkyl and (C₃-C₇)cycloalkyl-(C₁-C₆)alkyl, wherein said one or more (C₁-C₆)alkyl and (C₃-C₄)cycloalkyl-(C₁-C₂)alkyl is optionally substituted with one or more substituents independently selected from halogen, hydroxy, (C₁-C₄)alkoxy, (C₁-C₄)alkyl-S—, (C₁-C₄)alkyl-SO—, and (C₁-C₄)alkyl-SO₂—.

13. The compound according to of claim 1, wherein R₁ is pyrazol-3-yl or 1,2,3-triazol-4-yl substituted with one substituent selected from (C₁-C₄)alkyl and (C₃-C₄)cycloalkyl-(C₁-C₂)alkyl wherein said (C₁-C₄)alkyl and (C₃-C₄)cycloalkyl-(C₁-C₂)alkyl is optionally substituted with a substituent selected from halogen, hydroxy, (C₁-C₄)alkoxy, (C₁-C₄)alkyl-SO—, and (C₁-C₄)alkyl-SO₂—, R₂ is pyrazol-4-yl substituted with one or more (C₁-C₄)alkyl or deuterated (C₁-C₄)alkyl, R₃ is —CHR₅R₆, wherein each R₅ and R₆ is independently (C₃-C₇)cycloalkyl.

14. The compound according to claim 13, wherein R₁ is 2-(C₁-C₃)alkyl-pyrazol-3-yl, R₂ is 3,5-di(C₁-C₂)alkyl-pyrazol-4-yl, R₃ is —CHR₅R₆, wherein each R₅ and R₆ is independently (C₃-C₄)cycloalkyl.

15. The compound according to claim 1, wherein:
a) X is N and Y, Z, and V are independently selected from CH and C(R₄),
b) Y is N and X, 4 and V are independently selected from CH and C(R₄),
c) X and Y are N and V and Z are independently selected from CH and C(R₄),
d) Y and Z are N and X and V are independently selected from CH and C(R₄),
e) X and Z are N and Y and V are independently selected from CH and C(R₄), or
f) Y and V are N and X and Z are independently selected from CH and C(R₄).

16. The compound according to claim 1, wherein X is N, Y is C(R₄), and V and Z are CH.

17. The compound according to claim 1, wherein
R₁ is selected from pyrazolyl, imidazolyl, thiazolyl, isoxazolyl, oxadiazolyl, and triazolyl, wherein said pyrazolyl, imidazolyl, thiazolyl, isoxazolyl, oxadiazolyl, and triazolyl is optionally substituted with a substituent independently selected from Rₐ;
Rₐ is deuterium, halogen, hydroxy, —NR_{c}R_{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-SO₂—, and —NR_{c}R_{d};
R₂ is pyrazolyl or imidazolyl, wherein said pyrazolyl or imidazolyl is optionally substituted with one or more substituents independently selected from R_{b};
R_{b} is deuterium, halogen, cyano, hydroxy, —NR_{c}R_{d}, (C₁-C₆)alkyl, (C₁-C₆)alkoxy, (C₁-C₆)alkyl-CO—O—(CH₂)ₙ—, or (C₃-C₇)cycloalkyl, wherein n is 1-4, and wherein said (C₁-C₆)alkyl, (C₁-C₆)alkoxy, or (C₃-C₇)cycloalkyl is optionally substituted with one or more substituents independently selected from deuterium, halogen, cyano, hydroxy, —NR_{c}R_{d}, and (C₁-C₄)alkoxy;
each R_{c} and R_{d} is independently selected from the group consisting of hydrogen and (C₁-C₆)alkyl, or R_{c} and R_{d} together form azetidinyl, pyrrolidinyl, or piperidinyl, wherein said (C₁-C₆)alkyl, azetidinyl, pyrrolidinyl, or piperidinyl is optionally substituted with one or more substituents independently selected from halogen, cyano, and hydroxy;
L is a bond or —CHR_{g}O—,
R_{g} is hydrogen or (C₁-C₆)alkyl;
R₃ is —CHR₅R₆, and wherein each R₅ and R₆ is independently (C₃-C₇)cycloalkyl, wherein said (C₃-C₇)cycloalkyl is optionally substituted with one or more substituents independently selected from halogen, cyano, and (C₁-C₄)alkyl;
X is N, Y is C(R₄), and V and Z are CH;
R₄ is selected from (C₁-C₆)alkyl, (C₁-C₆)alkoxy, hydroxy, NH₂, and halogen;
wherein said (C₁-C₆)alkyl and (C₁-C₆)alkoxy may optionally be substituted with one or more halogens;
or a pharmaceutically acceptable salt, hydrate, or solvate thereof.

18. The compound according to claim 17, wherein R₁ is pyrazol-3-yl or 1,2,3-triazol-4-yl substituted with one or more (C₁-C₄)alkyl or (C₃-C₄)cycloalkyl-(C₁-C₂)alkyl, wherein said (C₁-C₄)alkyl or (C₃-C₄)cycloalkyl-(C₁-C₂)alkyl may optionally be substituted with one or more substituents selected from halogen, (C₁-C₄)alkoxy, (C₁-C₄)alkyl-SO₂,
R₂ is pyrazol-4-yl substituted with one or more (C₁-C₄)alkyl, or deuterated (C₁-C₄)alkyl,
R₃ is —CHR₅R₆, wherein each R₅ and R₆ is independently (C₃-C₇)cycloalkyl, and
X is N, Y is C(R₄), wherein R₄ is halogen, and V and Z are CH.

19. The compound according to claim 18, wherein R₁ is 2-(C₁-C₃)alkyl-pyrazol-3-yl, R₂ is 3,5-di(C₁-C₂)alkyl-pyrazol-4-yl, R₃ is —CHR₅R₆, and wherein each R₅ and R₆ independently is (C₃-C₄)cycloalkyl, and X is N, Y is C(R₄), wherein R₄ is fluoro, and V and Z are CH.

20. The compound according to claim 1 selected from
i) N-[(1R)-1-(dicyclopropylmethyl)-2-[[5-(3,5-dimethyl-1H-pyrazol-4-yl)-3-fluoro-2-pyridyl]amino]-2-oxo-ethyl]-2-ethyl-pyrazole-3-carboxamide;
ii) N-[(1R)-1-(dicyclopropylmethyl)-2-[[5-(3,5-dimethyl-1H-pyrazol-4-yl)-6-fluoro-2-pyridyl]amino]-2-oxo-ethyl]-2-ethyl-pyrazole-3-carboxamide;
iii) N-[(1R)-1-(dicyclopropylmethyl)-2-[[5-(3,5-dimethyl-1H-pyrazol-4-yl)-6-methoxy-2-pyridyl]amino]-2-oxo-ethyl]-2-ethyl-pyrazole-3-carboxamide;
iv) N-[(1R)-1-(dicyclopropylmethyl)-2-[[5-(3,5-dimethyl-1H-pyrazol-4-yl)-6-methyl-2-pyridyl]amino]-2-oxo-ethyl]-2-ethyl-pyrazole-3-carboxamide;
v) N-[(1R)-2-[[6-(3,5-dimethyl-1H-pyrazol-4-yl)-3-pyridyl]amino]-1-(4-methylcyclohexyl)-2-oxo-ethyl]-2-isopropyl-pyrazole-3-carboxamide;
vi) N-[(1S)-1-(dicyclopropylmethyl)-2-[[2-(3,5-dimethyl-1H-pyrazol-4-yl)pyrimidin-5-yl]amino]-2-oxo-ethyl]-2-ethyl-pyrazole-3-carboxamide;
vii) N-[(1S)-1-(dicyclopropylmethyl)-2-[[5-(3,5-dimethyl-1H-pyrazol-4-yl)-3-fluoro-2-pyridyl]amino]-2-oxo-ethyl]-2-ethyl-pyrazole-3-carboxamide;
viii) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(3-hydroxypropyl)pyrazole-3-carboxamide;
ix) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-ethyl-pyrazole-3-carboxamide;
x) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-isopropyl-pyrazole-3-carboxamide;
xi) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-methyl-pyrazole-3-carboxamide;
xii) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-ethyl-isoxazole-4-carboxamide;
xiii) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-ethyl-triazole-4-carboxamide;
xiv) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-isopropyl-isoxazole-4-carboxamide;
xv) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-isopropyl-triazole-4-carboxamide;
xvi) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-methyl-isoxazole-4-carboxamide;
xvii) N-[(1S)-1-(dicyclopropylmethyl)-2-[[5-(3,5-dimethyl-1H-pyrazol-4-yl)-6-fluoro-2-pyridyl]amino]-2-oxo-ethyl]-5-methyl-1-tetrahydropyran-4-yl-pyrazole-4-carboxamide;
xviii) N-[(1S)-1-(dicyclopropylmethyl)-2-[[5-(3,5-dimethyl-1H-pyrazol-4-yl)-6-methoxy-2-pyridyl]amino]-2-oxo-ethyl]-2-ethyl-pyrazole-3-carboxamide;
xix) N-[(1S)-1-(dicyclopropylmethyl)-2-[[5-(3,5-dimethyl-1H-pyrazol-4-yl)-6-methyl-2-pyridyl]amino]-2-oxo-ethyl]-2-ethyl-pyrazole-3-carboxamide;
xx) N-[(1S)-1-(dicyclopropylmethyl)-2-[[5-(3,5-dimethyl-1H-pyrazol-4-yl)pyrazin-2-yl]amino]-2-oxo-ethyl]-2-ethyl-pyrazole-3-carboxamide;
xxi) N-[(1S)-1-(dicyclopropylmethyl)-2-[[5-(3,5-dimethyl-1H-pyrazol-4-yl)pyrimidin-2-yl]amino]-2-oxo-ethyl]-2-ethyl-pyrazole-3-carboxamide;
xxii) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-2-(3-hydroxypropyl)pyrazole-3-carboxamide;
xxiii) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-2-methyl-pyrazole-3-carboxamide;
xxiv) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-3-ethyl-triazole-4-carboxamide;
xxv) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-3-isopropyl-triazole-4-carboxamide;
xvi) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-5-methyl-1-tetrahydropyran-4-yl-pyrazole-4-carboxamide;
xxvii) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-2-ethyl-pyrazole-3-carboxamide;
xxviii) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-2-isopropyl-pyrazole-3-carboxamide;
xxix) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-3-ethyl-isoxazole-4-carboxamide;
xxx) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-3-isopropyl-isoxazole-4-carboxamide;
xxxi) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-3-methyl-isoxazole-4-carboxamide;
xxii) N-[(1S)-1-(dicyclopropylmethyl)-2-[[6-(3,5-dimethyl-1H-pyrazol-4-yl)-3-pyridyl]amino]-2-oxo-ethyl]-2-isopropyl-pyrazole-3-carboxamide;
xxxiii) N-[(1S)-1-(dicyclopropylmethyl)-2-[[6-(3,5-dimethyl-1H-pyrazol-4-yl)-5-methoxy-3-pyridyl]amino]-2-oxo-ethyl]-2-ethyl-pyrazole-3-carboxamide;
xxxiv) N-[(1S)-1-(dicyclopropylmethyl)-2-[[6-(3,5-dimethyl-1H-pyrazol-4-yl)-5-methyl-3-pyridyl]amino]-2-oxo-ethyl]-2-ethyl-pyrazole-3-carboxamide;
xxxv) N-[(1S)-1-(dicyclopropylmethyl)-2-[[6-(3,5-dimethyl-1H-pyrazol-4-yl)pyridazin-3-yl]amino]-2-oxo-ethyl]-2-ethyl-pyrazole-3-carboxamide;
xxxvi) N-[(1S)-1-(dicyclopropylmethyl)-2-[[6-(5-ethyl-3-methyl-1H-pyrazol-4-yl)-3-pyridyl]amino]-2-oxo-ethyl]-2-(2-methoxyethyl)pyrazole-3-carboxamide;
xxxvii) N-[(1S)-1-(dicyclopropylmethyl)-2-[[6-(5-ethyl-3-methyl-1H-pyrazol-4-yl)-3-pyridyl]amino]-2-oxo-ethyl]-2-(3-methoxypropyl)pyrazole-3-carboxamide;
xxxviii) N-[(1S)-1-(dicyclopropylmethyl)-2-[[6-(5-ethyl-3-methyl-1H-pyrazol-4-yl)-3-pyridyl]amino]-2-oxo-ethyl]-2-ethyl-pyrazole-3-carboxamide;
xxxix) N-[(1S)-1-(dicyclopropylmethyl)-2-[[6-(5-ethyl-3-methyl-1H-pyrazol-4-yl)-3-pyridyl]amino]-2-oxo-ethyl]-2-isopropyl-pyrazole-3-carboxamide;
xl) N-[(1S)-1-cyclohexyl-2-[[5-(3,5-dimethyl-1H-pyrazol-4-yl)-2-pyridyl]amino]-2-oxo-ethyl]-2-isopropyl-pyrazole-3-carboxamide;
xli) N-[(1S)-1-cyclohexyl-2-[[6-(3,5-dimethyl-1H-pyrazol-4-yl)-3-pyridyl]amino]-2-oxo-ethyl]-2-isopropyl-pyrazole-3-carboxamide;
xlii) N-[(1S)-2-[[5-(3,5-dimethyl-1H-pyrazol-4-yl)-2-pyridyl]amino]-1-(4-methylcyclohexyl)-2-oxo-ethyl]-2-isopropyl-pyrazole-3-carboxamide;
xliii) N-[(1S)-2-[[6-(3,5-dimethyl-1H-pyrazol-4-yl)-3-pyridyl]amino]-1-(4-methylcyclohexyl)-2-oxo-ethyl]-2-isopropyl-pyrazole-3-carboxamide;
xliv) N-[1-(dicyclopropylmethyl)-2-[[5-(3,5-dimethyl-1H-pyrazol-4-yl)-3-fluoro-2-pyridyl]amino]-2-oxo-ethyl]-2-ethyl-pyrazole-3-carboxamide;
xlv) N-[1-(dicyclopropylmethyl)-2-[[5-(3,5-dimethyl-1H-pyrazol-4-yl)-3-methoxy-2-pyridyl]amino]-2-oxo-ethyl]-2-ethyl-pyrazole-3-carboxamide;
xlvi) N-[1-(dicyclopropylmethyl)-2-[[5-(3,5-dimethyl-1H-pyrazol-4-yl)-4-fluoro-2-pyridyl]amino]-2-oxo-ethyl]-2-ethyl-pyrazole-3-carboxamide;
xlvii) N-[1-(dicyclopropylmethyl)-2-[[5-(3,5-dimethyl-1H-pyrazol-4-yl)-4-methoxy-2-pyridyl]amino]-2-oxo-ethyl]-2-ethyl-pyrazole-3-carboxamide;
xlviii) N-[1-(dicyclopropylmethyl)-2-[[5-(3,5-dimethyl-1H-pyrazol-4-yl)-6-methoxy-2-pyridyl]amino]-2-oxo-ethyl]-2-ethyl-pyrazole-3-carboxamide;
xlix) N-[1-(dicyclopropylmethyl)-2-[[5-(3,5-dimethyl-1H-pyrazol-4-yl)-6-methyl-2-pyridyl]amino]-2-oxo-ethyl]-2-ethyl-pyrazole-3-carboxamide;
l) N-[1-(dicyclopropylmethyl)-2-[[5-(5-ethyl-3-methyl-1H-pyrazol-4-yl)-2-pyridyl]amino]-2-oxo-ethyl]-2-(2-methoxyethyl)pyrazole-3-carboxamide;
lj) N-[1-(dicyclopropylmethyl)-2-[[5-(5-ethyl-3-methyl-1H-pyrazol-4-yl)-2-pyridyl]amino]-2-oxo-ethyl]-2-(3-methoxypropyl)pyrazole-3-carboxamide;
lii) N-[1-(dicyclopropylmethyl)-2-[[5-(5-ethyl-3-methyl-1H-pyrazol-4-yl)-2-pyridyl]amino]-2-oxo-ethyl]-2-ethyl-pyrazole-3-carboxamide;
liii) N-[1-(dicyclopropylmethyl)-2-[[5-(5-ethyl-3-methyl-1H-pyrazol-4-yl)-2-pyridyl]amino]-2-oxo-ethyl]-2-isopropyl-pyrazole-3-carboxamide;
liv) N-[1-(dicyclopropylmethyl)-2-[[5-(5-ethyl-3-methyl-1H-pyrazol-4-yl)-6-fluoro-2-pyridyl]amino]-2-oxo-ethyl]-3-ethyl-isoxazole-4-carboxamide;
lv) N-[1-(dicyclopropylmethyl)-2-[[5-(5-ethyl-3-methyl-1H-pyrazol-4-yl)-6-fluoro-2-pyridyl]amino]-2-oxo-ethyl]-3-isopropyl-isoxazole-4-carboxamide;
lvi) N-[1-(dicyclopropylmethyl)-2-[[5-(5-ethyl-3-methyl-1H-pyrazol-4-yl)-6-fluoro-2-pyridyl]amino]-2-oxo-ethyl]-3-methyl-isoxazole-4-carboxamide;
lvii) N-[1-(dicyclopropylmethyl)-2-[[6-(3,5-dimethyl-1H-pyrazol-4-yl)-2-fluoro-3-pyridyl]amino]-2-oxo-ethyl]-2-ethyl-pyrazole-3-carboxamide;
viii) N-[1-(dicyclopropylmethyl)-2-[[6-(3,5-dimethyl-1H-pyrazol-4-yl)-5-fluoro-3-pyridyl]amino]-2-oxo-ethyl]-2-ethyl-pyrazole-3-carboxamide;
lix) N-[(1S)-1-(dicyclopropylmethyl)-2-[[5-(3,5-dimethyl-1H-pyrazol-4-yl)-2-pyridyl]amino]-2-oxo-ethyl]-2-isopropyl-pyrazole-3-carboxamide;
lx) N-[(1R)-1-(dicyclopropylmethyl)-2-[[5-(3,5-dimethyl-1H-pyrazol-4-yl)-2-pyridyl]amino]-2-oxo-ethyl]-2-isopropyl-pyrazole-3-carboxamide;
lxi) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(4,4,4-trifluoro-3-hydroxy-butyl)pyrazole-3-carboxamide;
lxii) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-2-(4,4,4-trifluoro-3-hydroxy-butyl)pyrazole-3-carboxamide;
lxiii) N-[(1S)-1-[[6-chloro-5-(3,5-dimethyl-1H-pyrazol-4-yl)-2-pyridyl]carbamoyl]-2,2-dicyclopropyl-ethyl]-2-methyl-pyrazole-3-carboxamide;
lxiv) N-[(1S)-1-[[6-chloro-5-(3,5-dimethyl-1H-pyrazol-4-yl)-2-pyridyl]carbamoyl]-2,2-dicyclopropyl-ethyl]-2-ethyl-pyrazole-3-carboxamide;
lxv) N-[(1S)-1-[[6-chloro-5-(3,5-dimethyl-1H-pyrazol-4-yl)-2-pyridyl]carbamoyl]-2,2-dicyclopropyl-ethyl]-2-isopropyl-pyrazole-3-carboxamide;
lxvi) N-[(1S)-1-[[6-chloro-5-(3,5-dimethyl-1H-pyrazol-4-yl)-2-pyridyl]carbamoyl]-2,2-dicyclopropyl-ethyl]-3-methyl-isoxazole-4-carboxamide;
xvii) N-[(1S)-1-[[6-chloro-5-(3,5-dimethyl-1H-pyrazol-4-yl)-2-pyridyl]carbamoyl]-2,2-dicyclopropyl-ethyl]-3-ethyl-isoxazole-4-carboxamide;
xviii) N-[(1S)-1-[[6-chloro-5-(3,5-dimethyl-1H-pyrazol-4-yl)-2-pyridyl]carbamoyl]-2,2-dicyclopropyl-ethyl]-3-isopropyl-isoxazole-4-carboxamide;
lxix) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-propyl-pyrazole-3-carboxamide;
lxx) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(2-methylsulfanylethyl)pyrazole-3-carboxamide;
lxxi) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(2-methylsulfinylethyl)pyrazole-3-carboxamide;
lxxii) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(2-methylsulfonylethyl)pyrazole-3-carboxamide;
lxxiii) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(3-methylsulfanylpropyl)pyrazole-3-carboxamide;
lxxiv) N-[(1R)-1-(dicyclopropylmethyl)-2-[[5-(3,5-dimethyl-1H-pyrazol-4-yl)-6-fluoro-2-pyridyl]amino]-2-oxo-ethyl]-2-(3-methylsulfanylpropyl)pyrazole-3-carboxamide;
lxxv) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(3-methylsulfinylpropyl)pyrazole-3-carboxamide;
lxxvi) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(3-methylsulfonylpropyl)pyrazole-3-carboxamide;
lxxvii) N-[(1S)-1-(dicyclopropylmethyl)-2-[[5-(3-ethyl-5-methyl-1H-pyrazol-4-yl)-6-fluoro-2-pyridyl]amino]-2-oxo-ethyl]-2-propyl-pyrazole-3-carboxamide;
lxxviii) N-[(1S)-1-(dicyclopropylmethyl)-2-[[5-(3-ethyl-5-methyl-1H-pyrazol-4-yl)-6-fluoro-2-pyridyl]amino]-2-oxo-ethyl]-2-(2-methylsulfanylethyl)pyrazole-3-carboxamide;
lxxix) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-2-(2-methylsulfinylethyl)pyrazole-3-carboxamide;
lxxx) N-[(1S)-1-(dicyclopropylmethyl)-2-[[5-(3-ethyl-5-methyl-1H-pyrazol-4-yl)-6-fluoro-2-pyridyl]amino]-2-oxo-ethyl]-2-(2-methylsulfonylethyl)pyrazole-3-carboxamide;
lxxxi) N-[(1S)-1-(dicyclopropylmethyl)-2-[[5-(3-ethyl-5-methyl-1H-pyrazol-4-yl)-6-fluoro-2-pyridyl]amino]-2-oxo-ethyl]-2-(3-methylsulfanylpropyl)pyrazole-3-carboxamide;
lxxxii) N-[(1S)-1-(dicyclopropylmethyl)-2-[[5-(5-ethyl-3-methyl-1H-pyrazol-4-yl)-6-fluoro-2-pyridyl]amino]-2-oxo-ethyl]-2-(3-methylsulfinylpropyl)pyrazole-3-carboxamide;
lxxxiii) N-[(1S)-1-(dicyclopropylmethyl)-2-[[5-(3-ethyl-5-methyl-1H-pyrazol-4-yl)-6-fluoro-2-pyridyl]amino]-2-oxo-ethyl]-2-(3-methylsulfonylpropyl)pyrazole-3-carboxamide;
lxxxiv) N-[(1S)-1-(dicyclopropylmethyl)-2-[[5-(3,5-dimethyl-1H-pyrazol-4-yl)-6-fluoro-2-pyridyl]amino]-2-oxo-ethyl]-4-methyl-1,2,5-oxadiazole-3-carboxamide;
lxxxv) N-[(1S)-1-(dicyclopropylmethyl)-2-[[5-(3,5-dimethyl-1H-pyrazol-4-yl)-6-fluoro-2-pyridyl]amino]-2-oxo-ethyl]-4-ethyl-1,2,5-oxadiazole-3-carboxamide;
lxxxvi) N-[(1S)-1-(dicyclopropylmethyl)-2-[[5-(3,5-dimethyl-1H-pyrazol-4-yl)-6-fluoro-2-pyridyl]amino]-2-oxo-ethyl]-4-(3-hydroxypropyl)-1,2,5-oxadiazole-3-carboxamide;
lxxxvii) 4-cyclopropyl-N-[(1S)-1-(dicyclopropylmethyl)-2-[[5-(3,5-dimethyl-1H-pyrazol-4-yl)-6-fluoro-2-pyridyl]amino]-2-oxo-ethyl]-1,2,5-oxadiazole-3-carboxamide;
lxxxviii) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-propyl-triazole-4-carboxamide;
lxxxix) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-sec-butyl-triazole-4-carboxamide;
xc) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-[2-fluoro-1-(fluoromethyl)ethyl]triazole-4-carboxamide;
xci) N-[(1S)-1-(dicyclopropylmethyl)-2-[[5-(3,5-dimethyl-1H-pyrazol-4-yl)-6-fluoro-2-pyridyl]amino]-2-oxo-ethyl]-1-methyl-tetrazole-5-carboxamide;
xcii) 2-cyclopropyl-N-[(1S)-1-(dicyclopropylmethyl)-2-[[5-(3,5-dimethyl-1H-pyrazol-4-yl)-6-fluoro-2-pyridyl]amino]-2-oxo-ethyl]pyrazole-3-carboxamide;
xciii) 2-(cyclopropylmethyl)-N-[(1S)-1-(dicyclopropylmethyl)-2-[[5-(3,5-dimethyl-1H-pyrazol-4-yl)-6-fluoro-2-pyridyl]amino]-2-oxo-ethyl]pyrazole-3-carboxamide;
xciv) 2-(cyclobutylmethyl)-N-[(1S)-1-(dicyclopropylmethyl)-2-[[5-(3,5-dimethyl-1H-pyrazol-4-yl)-6-fluoro-2-pyridyl]amino]-2-oxo-ethyl]pyrazole-3-carboxamide;
xcv) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(3,3-difluoropropyl)pyrazole-3-carboxamide;
xcvi) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-[2-fluoro-1-(fluoromethyl)ethyl]pyrazole-3-carboxamide;
xcvii) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-[(3,3-difluorocyclobutyl)methyl]pyrazole-3-carboxamide;
xcviii) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-[(1-methylazetidin-3-yl)methyl]pyrazole-3-carboxamide;
xcix) 2-(azetidin-3-ylmethyl)-N-[(1S)-1-(dicyclopropylmethyl)-2-[[5-(3,5-dimethyl-1H-pyrazol-4-yl)-6-fluoro-2-pyridyl]amino]-2-oxo-ethyl]pyrazole-3-carboxamide;
c) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-[(1S)-2-hydroxy-1-methyl-ethyl]pyrazole-3-carboxamide;
ci) N-[(1S)-1-(dicyclopropylmethyl)-2-[[5-(3,5-dimethyl-1H-pyrazol-4-yl)-6-fluoro-2-pyridyl]amino]-2-oxo-ethyl]-4-fluoro-2-(3-hydroxypropyl)pyrazole-3-carboxamide;
cii) N-[(1S)-1-(dicyclopropylmethyl)-2-[[5-(3,5-dimethyl-1H-pyrazol-4-yl)-6-fluoro-2-pyridyl]amino]-2-oxo-ethyl]-3-[(1R)-1-hydroxyethyl]isoxazole-4-carboxamide;
ciii) (2S)-3,3-dicyclopropyl-2-[[2,2-difluoro-2-(6-methoxy-3-pyridyl)acetyl]amino]-N-[5-(3,5-dimethyl-1H-pyrazol-4-yl)-6-fluoro-2-pyridyl]propanamide;
civ) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(2-fluoro-1-methyl-ethyl)pyrazole-3-carboxamide;
cv) N-[(1S)-1-(dicyclopropylmethyl)-2-[[5-(3,5-dimethyl-1H-pyrazol-4-yl)-6-fluoro-2-pyridyl]amino]-2-oxo-ethyl]-2-(2,2-difluoro-1-methyl-ethyl)pyrazole-3-carboxamide;
cvi) N-[(1S)-1-(dicyclopropylmethyl)-2-[[5-(3,5-dimethylisoxazol-4-yl)-6-fluoro-2-pyridyl]amino]-2-oxo-ethyl]-2-ethyl-pyrazole-3-carboxamide;
cvii) N-[(1R)-1-(dicyclopropylmethyl)-2-[[5-(3,5-dimethylisoxazol-4-yl)-6-fluoro-2-pyridyl]amino]-2-oxo-ethyl]-2-ethyl-pyrazole-3-carboxamide;
cviii) N-[(1S)-1-(dicyclopropylmethyl)-2-[[5-(3,5-dimethyltriazol-4-yl)-6-fluoro-2-pyridyl]amino]-2-oxo-ethyl]-2-ethyl-pyrazole-3-carboxamide;
cix) N-[(1R)-1-(dicyclopropylmethyl)-2-[[5-(3,5-dimethyltriazol-4-yl)-6-fluoro-2-pyridyl]amino]-2-oxo-ethyl]-2-ethyl-pyrazole-3-carboxamide;
cx) N-[(1S)-1-(dicyclopropylmethyl)-2-[[5-(3,5-dimethyl-1H-pyrazol-4-yl)-4,6-difluoro-2-pyridyl]amino]-2-oxo-ethyl]-3-isopropyl-triazole-4-carboxamide;
cxi) N-[(1S)-1-(dicyclopropylmethyl)-2-[[5-(3,5-dimethyl-1H-pyrazol-4-yl)-4,6-difluoro-2-pyridyl]amino]-2-oxo-ethyl]-2-ethyl-pyrazole-3-carboxamide;
cxii) N-[(1S)-1-(dicyclopropylmethyl)-2-[[5-(3,5-dimethyl-1H-pyrazol-4-yl)-4,6-difluoro-2-pyridyl]amino]-2-oxo-ethyl]-4-ethyl-1,2,5-oxadiazole-3-carboxamide;
cxiii) N-[(1S)-1-(dicyclopropylmethyl)-2-[[5-(3,5-dimethyl-1H-pyrazol-4-yl)-4,6-difluoro-2-pyridyl]amino]-2-oxo-ethyl]-3-isopropyl-isoxazole-4-carboxamide;
cxiv) N-[(1S)-1-(dicyclopropylmethyl)-2-[[4-(difluoromethyl)-5-(3,5-dimethyl-1H-pyrazol-4-yl)-2-pyridyl]amino]-2-oxo-ethyl]-2-ethyl-pyrazole-3-carboxamide;
cxv) N-[(1S)-1-(dicyclopropylmethyl)-2-[[4-(difluoromethyl)-5-(3,5-dimethyl-1H-pyrazol-4-yl)-2-pyridyl]amino]-2-oxo-ethyl]-2-isopropyl-pyrazole-3-carboxamide;
cxvi) N-[(1S)-1-(dicyclopropylmethyl)-2-[[6-(difluoromethyl)-5-(3,5-dimethyl-1H-pyrazol-4-yl)-2-pyridyl]amino]-2-oxo-ethyl]-2-ethyl-pyrazole-3-carboxamide;
cxvii) N-[(1S)-1-(dicyclopropylmethyl)-2-[[6-(difluoromethyl)-5-(3,5-dimethyl-1H-pyrazol-4-yl)-2-pyridyl]amino]-2-oxo-ethyl]-2-isopropyl-pyrazole-3-carboxamide;
cxviii) N-[(1S)-1-[[6-amino-5-(3,5-dimethyl-1H-pyrazol-4-yl)-2-pyridyl]carbamoyl]-2,2-dicyclopropyl-ethyl]-2-ethyl-pyrazole-3-carboxamide;
cxix) N-[(1S)-1-[[4-amino-5-(3,5-dimethyl-1H-pyrazol-4-yl)-6-fluoro-2-pyridyl]carbamoyl]-2,2-dicyclopropyl-ethyl]-2-ethyl-pyrazole-3-carboxamide;
cxx) N-[(1S)-1-(dicyclopropylmethyl)-2-[[5-(3,5-dimethyl-1H-pyrazol-4-yl)-3-hydroxy-2-pyridyl]amino]-2-oxo-ethyl]-2-ethyl-pyrazole-3-carboxamide;
cxxi) N-[(1S)-1-(dicyclopropylmethyl)-2-[[5-(3,5-dimethyl-1H-pyrazol-4-yl)-3-hydroxy-2-pyridyl]amino]-2-oxo-ethyl]-2-isopropyl-pyrazole-3-carboxamide;
cxxii) N-[(1S)-1-(dicyclopropylmethyl)-2-[[6-(3,5-dimethyl-1H-pyrazol-4-yl)-5-hydroxy-3-pyridyl]amino]-2-oxo-ethyl]-2-ethyl-pyrazole-3-carboxamide;
cxxiii) N-[(1S)-1-(dicyclopropylmethyl)-2-[[6-(3,5-dimethyl-1H-pyrazol-4-yl)-5-hydroxy-3-pyridyl]amino]-2-oxo-ethyl]-2-isopropyl-pyrazole-3-carboxamide;
cxxiv) N-[(1S)-1-[[6-chloro-5-(3,5-dimethyl-1H-pyrazol-4-yl)-2-pyridyl]carbamoyl]-2,2-dicyclopropyl-ethyl]-3-isopropyl-triazole-4-carboxamide;
cxxv) N-[(1S)-1-(dicyclopropylmethyl)-2-[[6-fluoro-5-[5-methyl-3-(1,1,2,2,2-pentadeuterioethyl)-1H-pyrazol-4-yl]-2-pyridyl]amino]-2-oxo-ethyl]-3-isopropyl-isoxazole-4-carboxamide;
cxxvi) N-[(1S)-1-(dicyclopropylmethyl)-2-[[6-fluoro-5-[3-methyl-5-(1,1,2,2,2-pentadeuterioethyl)-1H-pyrazol-4-yl]-2-pyridyl]amino]-2-oxo-ethyl]-3-ethyl-isoxazole-4-carboxamide;
cxxvii) N-[(1S)-1-(dicyclopropylmethyl)-2-[[6-fluoro-5-[5-methyl-3-(1,1,2,2,2-pentadeuteroethyl)-1H-pyrazol-4-yl]-2-pyridyl]amino]-2-oxo-ethyl]-3-methyl-isoxazole-4-carboxamide;
cxxviii) N-[(1S)-1-(dicyclopropylmethyl)-2-[[6-fluoro-5-[3-methyl-5-(1,1,2,2,2-pentadeuteroethyl)-1H-pyrazol-4-yl]-2-pyridyl]amino]-2-oxo-ethyl]-2-ethyl-pyrazole-3-carboxamide;
cxxix) N-[(1S)-1-(dicyclopropylmethyl)-2-[[6-fluoro-5-[5-methyl-3-(1,1,2,2,2-pentadeuteroethyl)-1H-pyrazol-4-yl]-2-pyridyl]amino]-2-oxo-ethyl]-2-isopropyl-pyrazole-3-carboxamide;
cxxx) N-[(1S)-1-(dicyclopropylmethyl)-2-[[6-fluoro-5-[5-methyl-3-(1,1,2,2,2-pentadeuteroethyl)-1H-pyrazol-4-yl]-2-pyridyl]amino]-2-oxo-ethyl]-3-isopropyl-triazole-4-carboxamide;
cxxxi) N-[(1S)-2-[[5-(3,5-dimethyl-1H-pyrazol-4-yl)-6-fluoro-2-pyridyl]amino]-2-oxo-1-[(7S)-spiro[2.5]octan-7-yl]ethyl]-2-ethyl-pyrazole-3-carboxamide;
cxxxii) 2-ethyl-N-[(1S)-2-[[5-(3-ethyl-5-methyl-1H-pyrazol-4-yl)-6-fluoro-2-pyridyl]amino]-2-oxo-1-[(7S)-spiro[2.5]octan-7-yl]ethyl]pyrazole-3-carboxamide;
cxxxiii) N-[(1S)-2-[[5-(3,5-dimethyl-1H-pyrazol-4-yl)-6-fluoro-2-pyridyl]amino]-2-oxo-1-spiro[2.3]hexan-5-yl-ethyl]-2-ethyl-pyrazole-3-carboxamide;
cxxxiv) N-[(1S)-2-[[5-(3,5-dimethyl-1H-pyrazol-4-yl)-6-fluoro-2-pyridyl]amino]-1-(4-methylcyclohexyl)-2-oxo-ethyl]-3-methyl-isoxazole-4-carboxamide;
cxxxv) N-[(1S)-2-[[5-(3-ethyl-5-methyl-1H-pyrazol-4-yl)-6-fluoro-2-pyridyl]amino]-1-(4-methylcyclohexyl)-2-oxo-ethyl]-3-methyl-isoxazole-4-carboxamide;
cxxxvi) N-[(1S)-2-[[5-(3,5-dimethyl-1H-pyrazol-4-yl)-6-fluoro-2-pyridyl]amino]-1-(4-methylcyclohexyl)-2-oxo-ethyl]-3-ethyl-isoxazole-4-carboxamide;
cxxxvii) 3-ethyl-N-[(1S)-2-[[5-(3-ethyl-5-methyl-1H-pyrazol-4-yl)-6-fluoro-2-pyridyl]amino]-1-(4-methylcyclohexyl)-2-oxo-ethyl]isoxazole-4-carboxamide;
cxxxviii) N-[(1S)-2-[[5-(3,5-dimethyl-1H-pyrazol-4-yl)-6-fluoro-2-pyridyl]amino]-1-(4-methylcyclohexyl)-2-oxo-ethyl]-3-isopropyl-isoxazole-4-carboxamide;
cxxxix) N-[(1S)-2-[[5-(3-ethyl-5-methyl-1H-pyrazol-4-yl)-6-fluoro-2-pyridyl]amino]-1-(4-methylcyclohexyl)-2-oxo-ethyl]-3-isopropyl-isoxazole-4-carboxamide;
cxl) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-methyl-pyrazole-3-carboxamide;
cxli) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-methyl-pyrazole-3-carboxamide;
cxlii) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-ethyl-pyrazole-3-carboxamide;
cxliii) 2-ethyl-N-[(1S)-2-[[5-(5-ethyl-3-methyl-1H-pyrazol-4-yl)-6-fluoro-2-pyridyl]amino]-1-(4-methylcyclohexyl)-2-oxo-ethyl]pyrazole-3-carboxamide;
cxliv) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-isopropyl-pyrazole-3-carboxamide;
cxlv) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-isopropyl-pyrazole-3-carboxamide;
cxlvi) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-propyl-pyrazole-3-carboxamide;
cxlvii) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-propyl-pyrazole-3-carboxamide;
cxlviii) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-(2-methoxyethyl)pyrazole-3-carboxamide;
cxlix) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-(2-methoxyethyl)pyrazole-3-carboxamide;
cl) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-(3-methoxypropyl)pyrazole-3-carboxamide;
cli) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-(3-methoxypropyl)pyrazole-3-carboxamide;
clii) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-(3-hydroxypropyl)pyrazole-3-carboxamide;
cliii) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-(3-hydroxypropyl)pyrazole-3-carboxamide;
cliv) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-(2-methylsulfanylethyl)pyrazole-3-carboxamide;
clv) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-(2-methylsulfanylethyl)pyrazole-3-carboxamide;
clvi) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-(2-methylsulfinylethyl)pyrazole-3-carboxamide;
clvii) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-(2-methylsulfinylethyl)pyrazole-3-carboxamide;
clviii) N-[(1S)-2-[[5-(3,5-dimethyl-1H-pyrazol-4-yl)-6-fluoro-2-pyridyl]amino]-1-(4-methylcyclohexyl)-2-oxo-ethyl]-2-(2-methylsulfonylethyl)pyrazole-3-carboxamide;
clix) N-[(1S)-2-[[5-(5-ethyl-3-methyl-1H-pyrazol-4-yl)-6-fluoro-2-pyridyl]amino]-1-(4-methylcyclohexyl)-2-oxo-ethyl]-2-(2-methylsulfonylethyl)pyrazole-3-carboxamide;
clx) N-[(1S)-1-(dicyclopropylmethyl)-2-[[5-(3,5-dimethyl-1H-pyrazol-4-yl)-6-fluoro-2-pyridyl]amino]-2-oxo-ethyl]-4-hydroxy-benzofuran-3-carboxamide;
clxi) N-[(1S)-1-(dicyclopropylmethyl)-2-[[5-(3,5-dimethyl-1H-pyrazol-4-yl)-6-fluoro-2-pyridyl]amino]-2-oxo-ethyl]-5-hydroxy-benzofuran-3-carboxamide;
clxii) N-[(1S)-1-(dicyclopropylmethyl)-2-[4-(3,5-dimethyl-1H-pyrazol-4-yl)anilino]-2-oxo-ethyl]-2-(1-methyl-2-methylsulfanyl-ethyl)pyrazole-3-carboxamide;
clxiii) N-[(1S)-1-(dicyclopropylmethyl)-2-[4-(3,5-dimethyl-1H-pyrazol-4-yl)anilino]-2-oxo-ethyl]-2-[(1-methylazetidin-3-yl)methyl]pyrazole-3-carboxamide;
clxiv) 2-(azetidin-3-ylmethyl)-N-[(1S)-1-(dicyclopropylmethyl)-2-[4-(3,5-dimethyl-1H-pyrazol-4-yl)anilino]-2-oxo-ethyl]pyrazole-3-carboxamide;
clxv) N-[(1S)-1-(dicyclopropylmethyl)-2-[4-(3,5-dimethyl-1H-pyrazol-4-yl)anilino]-2-oxo-ethyl]-2-(oxetan-3-ylmethyl)pyrazole-3-carboxamide;
clxvi) N-[(1S)-1-(dicyclopropylmethyl)-2-[4-(3,5-dimethyl-1H-pyrazol-4-yl)anilino]-2-oxo-ethyl]-2-(1-methyl-2-methylsulfinyl-ethyl)pyrazole-3-carboxamide;
clxvii) N-[(1S)-1-(dicyclopropylmethyl)-2-[4-(3,5-dimethyl-1H-pyrazol-4-yl)anilino]-2-oxo-ethyl]-2-(2-methylsulfanylethyl)pyrazole-3-carboxamide;
clxviii) N-[(1S)-1-(dicyclopropylmethyl)-2-[4-(3,5-dimethyl-1H-pyrazol-4-yl)anilino]-2-oxo-ethyl]-2-(2-methylsulfinylethyl)pyrazole-3-carboxamide;
clxix) N-[(1S)-1-(dicyclopropylmethyl)-2-[4-(3,5-dimethyl-1H-pyrazol-4-yl)anilino]-2-oxo-ethyl]-2-(3-methylsulfanylpropyl)pyrazole-3-carboxamide;
clxx) N-[(1S)-1-(dicyclopropylmethyl)-2-[4-(3,5-dimethyl-1H-pyrazol-4-yl)anilino]-2-oxo-ethyl]-2-(3-methylsulfinylpropyl)pyrazole-3-carboxamide;
clxxi) N-[(1S)-2-[4-(3,5-dimethyl-1H-pyrazol-4-yl)anilino]-1-(4-methylcyclohexyl)-2-oxo-ethyl]-2-(2-methylsulfanylethyl)pyrazole-3-carboxamide;
clxxii) N-[(1S)-2-[4-(3,5-dimethyl-1H-pyrazol-4-yl)anilino]-1-(4-methylcyclohexyl)-2-oxo-ethyl]-2-(2-methylsulfinylethyl)pyrazole-3-carboxamide;
clxxiii) N-[(1S)-2-[4-(3,5-dimethyl-1H-pyrazol-4-yl)anilino]-1-(4-methylcyclohexyl)-2-oxo-ethyl]-2-(3-methylsulfanylpropyl)pyrazole-3-carboxamide;
clxxiv) N-[(1S)-2-[4-(3,5-dimethyl-1H-pyrazol-4-yl)anilino]-1-(4-methylcyclohexyl)-2-oxo-ethyl]-2-(3-methylsulfinylpropyl)pyrazole-3-carboxamide;
clxxv) N-[(1S,2R)-2-cyclopropyl-1-[[4-(3,5-dimethyl-1H-pyrazol-4-yl)phenyl]carbamoyl]-3-(1-methylcyclopropyl)propyl]-2-ethyl-pyrazole-3-carboxamide;
clxxvi) N-[(1S,2S)-1-[[4-(3,5-dimethyl-1H-pyrazol-4-yl)phenyl]carbamoyl]-2-methyl-3-(1-methylcyclopropyl)propyl]-2-ethyl-pyrazole-3-carboxamide; or
clxxvii) N-[(1S)-1-(dicyclopropylmethyl)-2-[[5-(3,5-dimethyl-1H-pyrazol-4-yl)-6-(trifluoromethyl)-2-pyridyl]amino]-2-oxo-ethyl]-2-ethyl-pyrazole-3-carboxamide;
or a pharmaceutically acceptable salt, hydrate, or solvate thereof.

21-24. (canceled)

25. A method of treating psoriasis, ankylosing spondylitis, spondyloarthritis, or psoriatic arthritis in a subject in need thereof, comprising administering to the subject a therapeutically effective amount of a compound according to claim 1.

26. A pharmaceutical composition comprising a compound according to claim 1 and one or more pharmaceutically acceptable vehicles, excipients, or pharmaceutically acceptable carriers.

27. (canceled)

28. A method of treating psoriasis, ankylosing spondylitis, spondyloarthritis, or psoriatic arthritis in a subject in need thereof, comprising administering to the subject a therapeutically effective amount of the pharmaceutical composition according to claim 26.
